(19) United States
(12) Patent Application Publication
(10) Pub. No.: US 2014/0018247 A1
(43) Pub. Date: Jan. 16, 2014

(54) AMIS/UREI UREA-CHANNEL SUPERFAMILY CRYSTAL STRUCTURES

(71) Applicants: Hartmut Leucke, Irvine, CA (US); Reginald McNulty, Ladera Ranch, CA (US); George Sachs, Encino, CA (US)

(72) Inventors: Hartmut Leucke, Irvine, CA (US); Reginald McNulty, Ladera Ranch, CA (US); George Sachs, Encino, CA (US)

(21) Appl. No.: 13/868,700

(22) Filed: Apr. 23, 2013

Related U.S. Application Data
(60) Provisional application No. 61/637,754, filed on Apr. 24, 2012.

(51) Int. Cl. C07K 14/195; G06F 19/16

(52) U.S. Cl. CPC C07K 14/195; G06F 19/16
USPC 506/8; 530/350

(57) ABSTRACT

Embodiments of the inventions provide crystals of AmiS/Urel superfamily, urea-channel protein, methods of crystallizing such protein, and crystalline structures of such protein obtained by x-ray diffraction. Embodiments of the invention provide methods of identifying or designing antagonists and/or agonists of AmiS/Urel-superfamily, urea-channel protein based on crystalline structures thereof. In certain embodiments of the invention, the AmiS/Urel-superfamily, urea-channel protein is the Helicobacter pylori protein, HpUreI.

### PRIORITY DATA

The present application claims the benefit of U.S. Provisional Patent Application Ser. No. 61/637,754, which is hereby incorporated by reference in its entirety and which was filed Apr. 24, 2012.

This invention was made in part with United States Government support under Grant No. R01A178000, awarded by the National Institutes of Health. The U.S. Government has certain rights in this invention.

### FIELD OF THE INVENTIONS

Embodiments of the inventions relate to crystals of urea-channel proteins of the AmiS/Urel superfamily, methods of crystallizing such proteins, and crystal structures of such proteins obtained by x-ray diffraction. Embodiments of the invention relate to methods of identifying and/or designing ligands having antagonist or agonist activity on AmiS/Urel-superfamily, urea-channel proteins based on crystal structures thereof. In certain embodiments of the invention, the AmiS/Urel-superfamily protein is the Helicobacter pylori, proton-gated, urea-channel HpUreI.

### BACKGROUND OF THE INVENTIONS

Approximately 50% of the world's human population is chronically infected with the pathogenic bacterium, Helicobacter pylori (H. pylori) (R. E. Pounder and D. Ng, Aliment. Pharmacol. Ther. 9, 33 (1995)). At clinically significant rates, infection of the mammalian gastrointestinal tract by H. Pylori leads to gastric inflammation, ulcers, and gastric cancer (R. M. Peek and M. J. Blaser, Nature Rev. Cancer 2, 28 (2002)). The World Health Organization estimates that 750,000 deaths per year are caused by stomach cancer worldwide, and 90% or more of those are thought due to H. pylori infection. Current therapies for eradicating H. Pylori infection in humans include combination therapies of proton pump inhibitors and one or more antibiotics. Antibiotic based therapies are becoming less effective due to the development of H. Pylori antibiotic resistance. Accordingly, new therapeutic compounds for H. pylori infection are needed.

Survival of H. pylori in the acidic regions of the mammalian gastrointestinal tract (GI tract), such as the stomach and duodenum, depends on the presence of the inner membrane protein HpUreI (S. Skouloubris et al., Infect. Immun. 66, 4517 (1998)). HpUreI is an AmiS/Urel-superfamily, proton-gated urea channel that enables entry of urea into the cytoplasm of the bacterium. HpUreI is closed at pH 7.0, open below pH 4.5, and has a half maximum activation at pH 5.9 (D. L. Weeks et al., Science 287, 482 (2000)). Once in the H. Pylori cytoplasm, urea is hydrolyzed by urease to produce NH₃ and CO₂, which then travel into the periplasm, buffering it to a pH of 6.1 and enabling H. pylori to survive and colonize acidic regions of the mammalian GI tract (D. R. Scott et al., Gastroenterology 123, 187 (2002)). In addition, the closure of HpUreI at neutral pH prevents over-alkalization and cell death (K. Meyer-Rosberg, et al., Gastroenterology 111, 886 (1996)).

### SUMMARY OF THE INVENTIONS

A targeted approach, based on understanding a H. pylori, GI tract survival mechanism, would provide a means for identifying and/or designing new therapeutic agents for H. pylori infection (D. Y. Graham and L. Fischbach, Gut 59, 1143 (2010)). Along these lines, certain embodiments of the present invention provide crystals of the AmiS/Urel-superfamily, proton-gated, urea-channel protein, HpUreI. Certain embodiments of the present invention provide the crystal structure of HpUreI determined by multiwavelength anomalous diffraction (MAD) phasing and refinement to 3.25 Å resolution, useful in the identification and/or rational design of HpUreI ligands, and binding sites therefore, that modulate HpUreI urea channel activity.

Certain embodiments of the present invention provide crystals that comprise HpUreI protein having atomic coordinates that: correspond, at least at a 75% level, to those set forth in Table 4 or 5, are substantially identical to those set forth in Table 4 or 5, or are identical to those set forth in Table 4 or 5. Such crystals can be characterized by having approximately the following cell constants: a=123 Å, b=123 Å, c=135-152 Å and a space group of P4₂2₁2.

Certain embodiments of the present invention provide methods for identifying a ligand of HpUreI protein. Such methods can involve searching a molecular structure library with at least a portion of a crystal structure of HpUreI protein; and identifying, from the molecular structure library, a ligand for the HpUreI protein that has a 3-dimensional structure that interacts, in an operatively fitting manner, with the crystal structure. The energetics of the interaction can promote ligand binding to the HpUreI protein in a manner that modulates a urea channel activity of the HpUreI protein. The HpUreI crystal structure used for searching the molecular structure library can comprise atomic coordinates of the HpUreI protein that: correspond, at least at a 75% level, to those set forth in Table 4 or 5; are substantially identical to those set forth in Table 4 or 5; or are identical to those set forth in Table 4 or 5. The HpUreI urea channel modulation effected by HpUreI ligand binding can be a decrease or an increase in urea channel activity. And a ligand can bind HpUreI protein with an affinity selected from the group of ranges consisting of millimolar, micromolar, nanomolar, picomolar, and femtomolar.

In some embodiments, molecular structure libraries can comprise different types of molecules, such as small molecules, antibodies, peptides, proteins, DNA sequences, and/or RNA sequences.

Certain embodiments of the invention provide methods for determining a 3-dimensional structure of a complex comprising a HpUreI ligand bound to a HpUreI protein in a crystal. Such methods involve determining, by x-ray diffraction, atomic coordinates of HpUreI ligand and HpUreI protein in the crystal. Such crystals can be characterized by having approximately the following cell constants: a=123 Å, b=123 Å, c=135-152 Å and a space group of P4₂2₁2. And atomic coordinates for such crystallized, ligand-bound HpUreI protein can correspond, at least at a 75% level, to those set forth in Table 4 or 5, can be substantially identical to those set forth in Table 4 or 5, and can be identical to those set forth in Table 4 or 5.

Certain embodiments of the invention provide methods for determining a crystal structure of a complex comprising a HpUreI ligand bound to a HpUreI protein. The methods involve determining, by x-ray diffraction, phases and atomic coordinates of a crystal consisting of HpUreI protein; determining, by x-ray diffraction, phases and atomic coordinates of a co-crystal comprising HpUreI ligand bound to HpUreI; combining the phases to calculate a new electron density map for the complex; and determining a crystal structure of at least a portion of the complex based on the electron density map. In some embodiments, the portion of the determined 3-dimensional structure comprises the HpUreI ligand. In some embodiments, the crystal consisting of HpUreI protein can have approximately the following cell constants: a=123 Å, b=123 Å, c=135-152 Å and a space group of P4₂2₁2. And the atomic coordinates for the HpUreI protein in the crystal can: correspond, at a 75% level, to those set forth in Table 4 or 5; be substantially identical to those set forth in Table 4 or 5; or be identical to those set forth in Table 4 or 5.

Certain embodiments of the present invention provide methods for forming an HpUreI protein crystal. The methods involve exposing a crystallization volume that comprises a precipitant solution and an HpUreI protein to conditions that promote HpUreI protein crystal formation; and forming an HpUreI protein crystal. The atomic coordinates of the HpUreI protein in the HpUreI protein crystal are determinable by x-ray diffraction and can correspond, at least at a 75% level, to those set forth in Table 4 or 5; be substantially identical to those set forth in Table 4 or 5; or be identical to those set forth in Table 4 or 5. In some embodiments, the HpUreI protein crystal comprises approximately the following cell constants: a=123 Å, b=123 Å, c=135-152 Å and a space group of P4₂2₁2.

### BRIEF DESCRIPTION OF THE DRAWINGS

FIG. 1 shows a crystal of HpUreI used to collect x-ray diffraction data, the crystal belonging to spacegroup P4₂2₁2 and having an approximate maximal dimension of 0.25 mm.

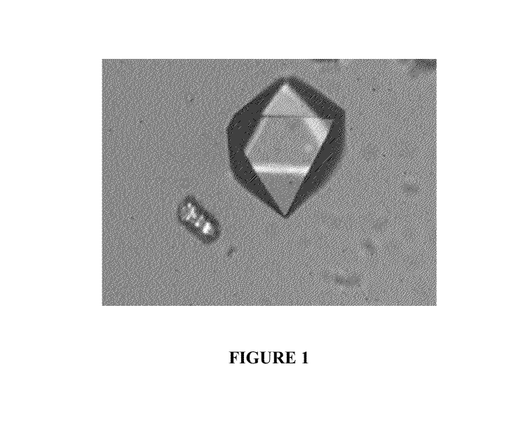

FIG. 2 shows a ribbon diagram of a crystal structure of the hexameric H. pylori urea channel protein, HpUreI, viewed from the periplasmic side and determined to a resolution of 3.25 Å using MAD and refinement.

FIG. 3 shows a side-view, ribbon diagram of one HpUreI protomer, which has an internal 3-fold pseudosymmetry resulting from a three-fold, tandem, and non-inverted repeat of a helical hairpin motif. FIG. 3 illustrates a HpUreI protomer overlaid on itself by rotations around an axis through the center of the channel pore by 120° and 240°. The root mean square deviations for pairwise overlays of the backbone atoms are 1.52 Å for the transmembrane helices (TMHs): TMH 1 and TMH2 onto TMH3 and TMH4; 1.13 Å for TMH3 and TMH4 onto TMH5 and TMH6; 1.76 Å for TMH5 and TMH6 onto TMH1 and TMH2, and 1.98 Å when all are superimposed simultaneously.

FIGS. 4A and 4B show ribbon diagrams of HpUreI protomers illustrating residues primarily lining the channel pore and highly conserved in the AmiS/Urel superfamily. FIG. 4A shows a view from the periplasm illustrating the open appearance of protomers B and C when only the helical backbone is illustrated. FIG. 4B shows a view parallel to membrane.

FIGS. 5A, 5B, and 5C show diagrams of the HpUreI channel pore. FIG. 5A shows a side view with periplasm on top, and illustrates the shape of the urea channel pore. FIG. 5B shows that a periplasmic constriction site is largely due to the side chains of Leu6, Val9, Phe84 and Trp149. FIG. 5C shows that a cytoplasmic constriction site is defined by the side chains of Leu13, Thr87, Tyr88, Leu152 and Trp153. In FIGS. 5B and 5C, TMHs 1-6 are labeled 1, 2, 3, 4, 5, and 6, respectively.

FIGS. 6A and 6B show ribbon diagrams of HpUreI hexamers with lipid tails at the center. FIG. 6A is a view from the periplasm. FIG. 6B is a side view with the periplasmic side on top, and illustrates 6 short lipid tails on the periplasmic side and 18 longer lipid tails on the cytoplasmic side.

FIG. 7 Shows electrostatic potential at the HpUreI hexamer surface, viewed from the periplasm and computed at pH 5.3. The HpUreI hexamer surface has been colored according to the electrostatic potential computed with an adaptive Poisson-Boltzmann algorithm, from −4 kT to +4 kT.

### DETAILED DESCRIPTION OF THE INVENTIONS

As used herein, the terms “AmiS/Urel-superfamily, urea-channel protein;” “HpUreI protein;” and “HpUreI urea channel” can be used interchangeably; and refer to the J99 HpUreI protein sequence (Supplementary FIG. 11 of Strugatsky, et al. Nature: volume 493, pages 255-258 (2013) doi:10.1038/naturell684), or a homolog thereof having at least 50%, 55%, 60%, 65%, 70%, 75%, 80%, 85%, 90%, 95%, or 97%, or 100% sequence identity to the H. Pylori J99 protein sequence.

The terms “ligand,” “drug,” and “compound” can be used interchangeably and refer to compounds that bind and/or physically associate with an AmiS/Urel-superfamily, urea-channel protein, such as HpUreI, and thereby modulate the protein's urea channel activity. Upon the binding of a ligand with an AmiS/Urel-superfamily, urea-channel protein, such as HpUreI, the activity of the protein's urea channel can be decreased or increased. The activity of a AmiS/Urel-superfamily, urea-channel protein's urea channel, as well as the modulations thereto induced by ligands, can be determined using a variety of techniques, including those described in Examples 1A and 1B. Ligands can comprise small molecules, antibodies, peptides, proteins, DNA sequences, RNA sequences, and combinations or derivatives thereof. Ligands can be commercially obtained or synthesized de novo. And ligands can be co-crystallized with an AmiS/Urel-superfamily, urea-channel protein of the invention, such as HpUreI, to determine the three-dimensional structure of a complex that comprises the protein bound to the co-crystallized ligand.

The terms “ligand binding site” and “binding site” can be used interchangeably, and refer to amino acid residues, or functional groups thereof, of an AmiS/Urel-superfamily, urea-channel protein of the invention, such as HpUreI, near the ligand in a complex that comprises the ligand bound to the urea channel protein. A ligand binding site can encompass the amino acid residues, or functional groups thereof, of the urea-channel protein that interact with the ligand. Such interactions include ion, hydrogen, van der Wals, hydrophobic, and covalent bonds. A ligand binding site can also encompass all of the amino acid residues within, for example, a 20 Å, 19 Å, 18 Å, 17 Å, 16 Å, 15 Å, 14 Å, 13 Å, 12 Å, 11 Å, or 10 Å radius of the ligand in a complex that comprises the ligand bound to a urea channel protein of the invention.

The terms “structural coordinates” and “atomic coordinates” can be used interchangeably, and refer to coordinates derived from mathematical equations related to patterns obtained on diffraction of a monochromatic beam of x-rays by the atoms (scattering centers) of a protein, or a complex comprising the protein, in crystal form. The diffraction data can be used to calculate an electron density map of the repeating units of the crystal. And an electron density map can be used to establish the positions of a protein's individual atoms within the unit cell of the crystal. Those skilled in the art will understand that such obtained data are dependent upon the particular system used, and hence, different coordinates may in fact describe an identical or equivalent crystal if such coordinates define substantially the same relationship as those described herein.

Tables 4 and 5 provide the atomic coordinates of a crystallized HpUreI protein protomer and trimer, respectively, of the invention. For purposes of this invention, any set of atomic coordinates of a crystallized AmiS/Urel-superfamily protein that have a root mean square deviation of about 3 Å or less when superimposed, using corresponding protein backbone atoms, on the atomic coordinates set forth in Tables 4 or 5 shall be considered substantially identical. Also for purposes of this invention, any set of atomic coordinates of a crystallized AmiS/Urel-superfamily protein that have a root mean square deviation of about 3 Å or less when at least 50%, 55%, 60%, 65%, 70%, 75%, 80%, 85%, 90%, 95%, or 97% of their corresponding backbone atoms are superimposed on the atomic coordinates set forth in Tables 4 or 5 shall be considered to correspond, at least at a 50% level, a 55% level, a 60% level, a 65% level, a 70% level, a 75% level, an 80% level, an 85% level, a 90% level, a 95% level, or a 97% level, respectively, to the atomic coordinates set forth in Tables 4 or 5. The term “backbone atoms” refers to all of the atoms present in amino acids that make up a protein, excluding the atoms present in the side chains of said amino acids.

The term “heavy-atom derivatization” refers to a method of producing a chemically modified form of a crystallized AmiS/Urel-superfamily, urea-channel protein of the invention, such as HpUreI. In practice, e.g., such a crystal is soaked in a solution containing heavy metal atom salts or organometallic compounds, such as lead chloride, gold thiomalate, thimerosal, selenium, or uranyl acetate, which can diffuse through the crystal and bind to the surface of a urea-channel protein therein. Also in practice, e.g., heavy metal atom derivatized amino acids, such as selonomethionine, may be incorporated into a recombinantly produced protein used to produce crystals for x-ray crystallography. The location of bound and/or incorporated heavy metal atom(s) can be determined by x-ray diffraction analysis, and this information can be used to generate phase information used to construct a three-dimensional structure of the so-derivatized, urea-channel protein in the crystal.

The term “unit cell” refers to a basic shaped block. The entire volume of a crystal may be constructed by regular assembly of such blocks. Each unit cell comprises a complete representation of the unit of pattern, the repetition of which builds up a crystal.

The term “spacegroup” refers to the arrangement of symmetry elements of a crystal.

The term “molecular replacement” refers to a method that involves generating a preliminary structural model of a protein crystal whose structural coordinates are unknown, by orienting and positioning a protein whose atomic coordinates are known, such as the HpUreI coordinates in Table 4 or 5, within the unit cell of the unknown protein crystal, so as to best account for the observed diffraction pattern of the unknown protein crystal. Phases can then be calculated from this model, and combined with the observed amplitudes to give an approximated Fourier synthesis of the structure whose coordinates are unknown. This in turn can be subject to any of the several forms of refinement to provide a final structure of the unknown protein crystal. (See, e.g., Lattman, E., “Use of the Rotation and Translation Functions,” Methods in Enzymology, 115: 55-77 (1985); Rossman, ed., “The Molecular Replacement Method,” Int. Sci. Rev. Ser. No. 13 (Gordon and Breach: New York, 1972)). Using the atomic coordinates of the AmiS/Urel-superfamily, urea-channel protein, HpUreI, provided by this invention, molecular replacement may be used to determine the atomic coordinates of a crystalline co-complex of HpUreI and a ligand, or a mutant, homolog, or different crystalline form of an AmiS/Urel-superfamily, urea-channel protein of the invention.

Certain embodiments of the invention provide methods for identifying ligands and ligand binding sites for AmiS/Urel-superfamily, urea-channel proteins, such as HpUreI. Such ligands can be designed de novo or identified by screening known molecules. In some embodiments, such ligands are useful pharmaceuticals; or prototypes useful for further pharmaceutical refinement (i.e., lead compounds) in order to improve binding affinity or other pharmacologically important features (e.g., bio-availability, toxicology, metabolism, pharmacokinetics). In some embodiments, such ligands interact with their binding sites on the urea-channel protein with a binding affinity in the micromolar, nanomolar, picomolar, or femtomolar range. In certain embodiments, ligands of the invention may be used alone, in combination with each other, or in combination with other agents in the treatment of human disease, such as H. pylori infection.

In certain embodiments of the invention, structural information for an AmiS/Urel-superfamily, urea-channel protein of the invention, such as HpUreI, obtained from atomic coordinates determined by crystallographic data is used to identify a ligand binding site on the protein, which can be used in the rational design of a ligand. For example, a rationally designed ligand of the invention can be developed by identifying the 3-dimensional arrangement of one or more surface-accessible feature(s) of a ligand binding site capable of supporting high affinity and/or specificity ligand binding. Such surface-accessible features include, for example, functional groups that comprise hydrogen bond donors, hydrogen bond acceptors, polar groups, charged groups, ionizable groups, acid groups, hydrophobic groups, and the like. The 3-dimensional arrangement of such surface-accessible features in the ligand binding site can be used to design ligands having their own functional group(s) oriented in three-dimensional space so as to operatively fit into the ligand binding site in a manner that facilitates energetically favorable interactions between the functional group(s) of the ligand and the surface-accessible feature(s) of the ligand binding site. Such energetically favorable interactions promote high affinity and/or specificity binding between a rationally designed ligand and its binding site, and include van der Waals contacts, electrostatic interactions, planar stacking, hydrogen bonding, and covalent bonding. The rational design of a ligand can further include consideration of the ligand's ability to achieve a low energy conformation compatible with high affinity and/or specificity binding of the ligand to its binding site.

Once functional groups that can interact with specific surface-accessible features in the ligand binding site have been identified, they can be linked in a single, rationally designed ligand using bridging fragments with appropriate size and geometry or frameworks which can support the functional groups at the above-described, energetically favorable orientations, thereby providing a rationally designed ligand according to the invention. Linking of functional groups in this way can be done manually or with the help of software such as QUANTA or SYBYL. By utilizing a fast docking program, individual compounds from, e.g., a compound database or library, can be evaluated for ligand site binding.

Processes for identifying a ligand binding site and the rational design of a ligand can vary. For example, a ligand binding site can be identified by visual inspection of surface accessible features by an expert. From such an inspection, corresponding ligands can be designed manually and/or with the assistance of compound database searching and/or computer modeling. In addition, computer programs may be employed to identify ligand binding sites and rationally design ligands.

Software packages for implementing molecular modeling techniques for use in rational ligand design include SYBYL (available from Tripos, Inc.); AMBER (available from Oxford Molecular); CERIUS2 (available from Molecular Simulations, Inc.); INSIGHT II (available from Molecular Simulations, Inc.); CATALYST (available from Molecular Simulations, Inc.); QUANTA (available from Molecular Simulations, Inc.); HYPERCHEM (available from Hypercube, Inc.); FIRST DISCOVERY (available from Schrodinger, Inc.), MOE (available from Chemical Computing Group), and CHEMSITE (available from Pyramid Learning).

In addition, a docking program may be utilized to evaluate individual ligands from, e.g., a compound database or library, for their predicted affinity and/or specificity of ligand site binding. Docking refers to a process in which two or more molecules are aligned based on energy considerations. Docking aligns the three-dimensional structures of two or more molecules to predict the conformation of a complex formed from the molecules. (See, e.g., Blaney and Dixon (1993) Perspectives in Drug Discovery and Design 1:301). Docking can be accomplished by either geometric matching of a ligand and its binding site or by minimizing the energy of interaction.

Suitable docking algorithms include DOCK (Kuntz et al. (1982) J. Mol. Biol. 161:269-288 and available from UCSF); AUTODOCK (Goodsell & Olson (1990) Proteins: Structure, Function and Genetics 8:195-202 and available from Oxford Molecular); MOE DOCK (Chemical Computing Group, Inc.); FLExX (Tripos, Inc.); GOLD (Jones et al. (1997) J. Mol. Biol. 267:727-748); AFFINITY (Molecular Simulations, Inc.); C2 LigandFit (Molecular Simulations, Inc.); DOCKIT (Metaphorics, LLC); and GLIDE (Schrodinger, Inc.).

Suitable structural libraries include the ACD, AsInEx, Bionet, ComGenex, the Derwent World Drug Index (WDI), the Contact Service Company database, LaboTest, ChemBridge Express Pick, ChemStar, BioByteMasterFile, Orion, SALOR, TRIAD, ILIAD, the National Cancer Institute database (NCl), and the Aldrich, Fluka, Sigma, and Maybridge catalogs.

Suitable de novo design software includes MCDLNG (Gehlhaar et al. (1995) J. Med. Chem. 38:466-72); MCSS/HOOK (Molecular Simulations Inc.); LUDI (Molecular Simulations Inc.); GROW (Moon and Howe (1991) Proteins: Str. Funct. Genet. 11:314-328); SPROUT; LEAPFROG (Tripos Inc.); GROUPBUILD (Rorstein et al. (1993) J. Med. Chem. 36:1700); CAVEAT (Lauri and Bartlett (1994) Comp. Aided Mol. Design. 8:51-66); and RASSE (Lai (1996) J. Chem. Inf. Comput. Sci. 36:1187-1194).

In certain embodiments of the invention, atomic coordinates of an AmiS/Urel-superfamily, urea-channel protein crystal structure can be stored on a medium for subsequent use with a computational device, such as a computer (e.g., supercomputer, mainframe, minicomputer, or microprocessor). Typically, the coordinates are stored on a medium configured to hold large amounts of data, such as magnetic or optical media (e.g., hard disks, compact disks, magneto-optical media (“floptical” disks or electronic media (e.g., random-access memory (RAM), or read-only memory (ROM). The storage medium can be local to the computer, or can be remote (e.g., a networked storage medium, including the Internet). The choice of computer, storage medium, networking, and other devices or techniques will be familiar to those of skill in the structural/computational chemistry arts. The atomic coordinates can be those noted in Table 4 or equivalents thereof.

The following examples further illustrate, without limitation, embodiments of the present invention.

### EXAMPLE 1

### Example 1A

### Oocyte HpUreI Urea Channel Activity Assay

A poly(A) cassette is cloned into pcDNA3.1(T7) (Invitrogen), into which the HpUreI gene sequence is inserted upstream of the poly(A) cassette and downstream of the T7 promoter. Capped RNA (cRNA) therefrom is prepared using the mMessage mMachine in vitro transcription system (Ambion). Then, 50 nl per oocyte of cRNA (1 μg/μl) is injected 2 days before urea uptake is measured (Hediger et al. 1987). Before each urea uptake experiment, the oocytes are maintained in Barth's solution (88 mM NaCl, 0.82 MgSO₄, 0.41 mM CaCl₂, 1 mM KCl, 0.33 mM Ca(NO₃)₂, 2.4 mM NaHCO₃, and 10 mM HEPES, pH 7.4) at 18° C. Urea uptake by oocytes is measured in Ringer's solution (100 mM NaCl₂, 2 mM KCl, 1 mM CaCl₂ and MgC12) buffered by 20 mM MES or 20 mM HEPES to pH 5.0 or 7.5, respectively, at 21° C. Oocytes are allowed to equilibrate in Ringer's solution buffered to pH 7.0 by 10 mM HEPES before transfer to the reaction buffer containing 50 uM [¹⁴C]-urea. Urea uptake reactions are terminated with the transfer of the oocytes to large quantities of urea-free buffer (pH 7.5) and, finally, to individual scintillation vials. Before the addition of scintillation fluid, oocytes are dissolved in 5% SDS. At least a single time point for the urea uptake reactions is taken after 30 min at pH 5.0 and 7.5, and urea uptake is assessed by an increase in the amount of incorporated [¹⁴C]-urea in injected oocytes compared with non-injected or oocytes.

### Example 1B

### Whole Cell H. pylori Urel Urea Channel Activity Assay

Urease activity is measured radiometrically. 10 μl of H. pylori bacterial suspension are added to 100 mM PB, pH 7.0 or pH 4.5 containing 5 mM [¹⁴C]-urea urea with a specific activity of 10 Plastic wells containing 0.5 M KOH-soaked filter paper hung from rubber stoppers are used to collect the total [¹⁴C]O₂ that resulted from the hydrolysis of urea by cytoplasmic urease. Urease activity is measured for 30 minutes at 37° C. with constant agitation. The reaction is terminated by the addition of 5 NH₂SO₄ and incubated 30 minutes at 37° C. The wells are placed in scintillation cocktail (HiIonicFluor, Packard Instruments, Meriden, Conn.), and the radioactivity is measured by scintillation counting (1216 RackBeta, LKB Inst.). Urease activity is calculated as μmoles urea hydrolyzed/min/mg protein. Protein concentration is determined by the BCA method.

### EXAMPLE 2

### Engineering a 6His Tag into HpUreI

DNA encoding HpUreI was isolated by PCR from Helicobacter pylori strain J99. A 6His tag was introduced into the protein at various locations to facilitate purification. The engineered proteins with a 6His tag at the N-terminus, in the first periplasmic loop (PL1), the second periplasmic loop (PL2) or at the C-terminus were expressed in Xenopus oocytes and tested for channel activity according to Example 1. Wild-type HpUreI showed urea uptake of 11.55+/−0.33 pmol/oocyte/6 min (n=3) and the PL1 6His tag showed an uptake of 3.0+/−0.5 pmol/oocyte/6 min (n=3). Mutants with the 6His insertion at the N terminus, in PL2 or at the C terminus were inactive. HpUreI with the 6His tag inserted in PL1 (HpUreI6HisPL1) was subsequently used for expression, purification, and crystallization.

### EXAMPLE 3

### HpUreI Expression and Membrane Isolation

pET101HpUreI6HisPL1 was transformed into E. coli C43 (Avidis S.A.). For small-scale expression and crude membrane isolation bacterial cultures were grown to 0.8 OD₆₀₀ and then induced by addition of 1 mM IPTG. After 3 hours of induction, cells were harvested by centrifugation at 3000 rpm for 10 min. The pellet was resuspended in a solution of 50 mM Na₂HPO₄ pH 7.4, 1 mM EDTA, 30 μg/ml DNase I and sonicated to lyse the cells. Cell debris was removed by centrifugation (10,000 g, 10 min) and membranes were collected (100,000 g, 45 min) and resuspended in the same buffer without DNase I (40-50 μl). Samples of membrane protein (25 μg, determined by BCA assay, Pierce Biotech., Inc.) were dissolved in gel loading buffer containing 1% mercaptoethanol and run without boiling on 4-12% SDS-polyacrylamide gels. After transfer to nitrocellulose, Western blot analysis was performed with either an anti-Urel or anti-His-tag antibody.

For large-scale membrane expression and subsequent crystallization trials, a bioreactor (BioFlo 110, New Brunswick) containing 10 l Luria Bertani broth supplemented with 50 mM K₂HPO₄ pH7.8 and 1.5% w/v glycerol was inoculated with 0.2 l overnight culture of the E. coli harboring a pET101HpUreI6HisPL1 plasmid. When the OD₆₀₀ reached 0.8-1.0 (˜2 hr), HpUreI expression was induced with 1 mM IPTG. Growth conditions were maintained at 37° C., with a continuous air supply at 5 liter/min and 500 rpm stirring for 5 hours until the cell density reached 5-7 OD₆₀₀. Cells were then collected by centrifugation and resuspended at 4° C. in 400 ml of buffer containing 50 mM Na₂HPO₄ pH 7.4, 30 μg/ml DNase, and 10 mM mercaptoethanol. All subsequent steps were carried out at 4° C. The bacterial suspension was passed twice through a French Press cell at 20,000 psi. The cell debris was removed (10,000 g, 20 min) and the remaining membranes were collected (100,000 g, 2 hr) to yield ˜4 grams of total membrane protein. The membrane pellet was resuspended in 350 ml of storage buffer (10 mM imidazole, 150 mM NaCl, 50 mM Na₂HPO₄ pH 7.4, 65 g/l glycerol, and 2 mM mercaptoethanol) to yield 10-12 mg/ml of membrane protein which was then flash frozen in liquid nitrogen and stored at −80° C.

### EXAMPLE 4

### Selenomethionine Labeling

The pET101UreI6HisPL1 construct was transformed into the methionine auxotroph strain BL21-CodonPlus-RP-X strain (Stratagene) for optimal labeling of HpUreI with selenomethionine in minimal medium. A 10 L culture of M9 minimal media (60 g/L Na₂HPO₄, 30 g/L KH₂PO₄, 10 g/L NH₄C1, 5 g/L NaCl, 0.4% w/v glucose, 0.1 mM CaCl₂, 2 mM MgSO₄, and 0.1 L of MEM vitamin solution (Mediatech) supplemented with 60 μg/ml selenomethionine, was inoculated with 200 ml overnight culture grown in the same minimal medium with 0.1 mg/ml methionine in place of selenomethionine. Protein expression was induced with 1 mM IPTG at OD₆₀₀ ˜0.8 and growth was continued for 40 h at 20° C.

### EXAMPLE 5

### HpUreI Purification

Membranes in storage buffer were collected at 100,000 g for 90 min, resuspended at 10 mg/ml in a buffer of 10 mM imidazole, 150 mM NaCl, 50 mM sodium phosphate, pH 7.4, and partially dissolved in 2% decylmaltoside (DM, Anatrace) by adding a 20% detergent solution drop wise and stirring on ice for 30 min. The insoluble material was pelleted (160,000 g, 30 min) and the supernate was gently agitated overnight with 5 ml of cobalt-agarose resin (TALON resin, Clontech) at 4° C. to bind the 6His-UreI. All the solutions used in subsequent rinsing and elution of the resin contained 150 mM NaCl, 50 mM sodium phosphate, pH 7.4, 0.2% DM and 0.1 mg/ml E. coli polar lipids (Avanti) with increasing concentrations of imidazole. The resin was rinsed with 10 volumes of buffer with 10 mM imidazole, then 30 volumes with 20 mM imidazole. HpUreI was then eluted with 30 ml of the same buffer with 250 mM imidazole. The eluted HpUreI was concentrated to ˜10 mg/ml by using Amicon 50 kDa filters prior to gel filtration.

HpUreI was purified on a Superosel2 column (10/300, GE) with buffer containing 10 mM MES pH 6.5, 150 mM NaCl; 0.2% DM; 0.1 mg/ml E. coli polar lipid extract. The peak fractions were pooled and concentrated to 10 mg/ml (50 kDa filters, Amicon) for use in crystallization trials.

### EXAMPLE 6

HpUreI Crystallization

Urel protein at 10 mg/ml in Superose12 column buffer was diluted to give a solution comprised of 1.57 mg/ml HpUreI protein, 40 mM NaCl, 1 mM TlC1, 10 mM CaCl₂, 7% PEG 400, 0.05% decylmaltoside, 2.25% octylglucoside, 0.8 mg/ml E. coli polar lipids (Avanti), 35 mM MES pH 5.3. This mixture (3.5 μl) was used for hanging drop diffusion over a reservoir (0.5 ml) of 20% PEG 400 in 0.1 M MES, pH 5.3. Crystals grew in 3-4 months at 11° C. and were dehydrated by raising the PEG 400 concentration in the reservoir in increments of 3% at two-day intervals until the final concentration in the well solution was 33% PEG 400.

### EXAMPLE 7

### X-Ray Data Collection and Data Reduction

Single crystals were mounted in nylon loops and flash cooled in liquid nitrogen. X-ray diffraction data collection was carried out at 100 K by collecting 180 diffraction images 1° in width. Data were integrated, scaled, and merged with the program XDS (W. Kabsch, Acta Cryst. D66, 125 (2010)) (Table 1). Due to variability of the c-axis length (135 to 152 Å), data could not be merged between multiple crystals. After diffraction data from hundreds of heavy metal-soaked crystals had been collected without yielding interpretable maps, selenomethionine (SeMet) phasing was employed. While fluorescence scans of the initially very small SeMet crystals indicated significant selenium incorporation, selenium sites could not be located from over 50 MAD and SAD datasets collected with 30° wedges. The successful MAD data set was collected from a rare larger crystal that took over five months to grow. Three-wavelengths MAD diffraction data were collected in 10° wedges at beamline 12-2 at SSRL (Table 1).

TABLE 1
Crystallographic data reduction & merging statistics

| | SeMet* | | | |
|---|---|---|---|---|
| | PEAK | HREM | INFL | Native |
| Spacegroup | P4₂2₁2 | P4₂2₁2 | P4₂2₁2 | P4₂2₁2 |
| Unit cell dimensions | | | | |
| a, b (□) | 123.3 | 123.3 | 123.3 | 122.9 |
| c (□) | 140.0 | 140.0 | 140.0 | 141.4 |
| Matthews coefficient (□³/Da) | 3.92 | 3.92 | 3.92 | 3.95 |
| No. of molecules in the | 3 | 3 | 3 | 3 |
| asymmetric unit | | | | |
| Solvent content (%) | 68.6 | 68.6 | 68.6 | 68.9 |
| Resolution (□) | 3.39 | 3.39 | 3.39 | 3.11 |
| Total observations | 513,360 | 610,548 | 511,938 | 107,320 |
| Unique reflections | 24,828* | 28,583* | 24,815* | 17,263 |
| Completeness (%) | 86.8 | 99.9 | 86.7 | 98.8 (97.9) |
| R_{merge} (%) | 21.7 | 24.2 | 22.6 | 5.0 (111) |
| Average I/sig(I) | 8.4 | 6.6 | 8.0 | 21.2 (16) |
| Mosaicity (□) | 0.25 | 0.27 | 0.26 | 0.33 |
| Average redundancy | 32.8 | 44.5 | 35.9 | 6.2 |

*With Bijvoet pairs not merged.

### EXAMPLE 8

SeMet Multiwavelength Anomalous Dispersion (MAD) Phasing

The program SHELXC (G. M. Sheldrick, Acta Cryst. D64, 112 (2008)) was used to determine the approximate resolution cutoff for the anomalous signal (when the correlation coefficient between the anomalous differences at the peak and remote wavelengths decreased to below 30%). SeMet sites were initially obtained using the program SHELXD (G. M. Sheldrick, Acta Cryst. D64, 112 (2008)). Of the nine possible sites (three protomers in the asymmetric unit with three SeMet sites per protomer, including the N-terminal SeMets), eight sites were located. From the symmetry of these eight sites, a ninth site could be located, leading to the localization of all anomalous scatterers. The boundary of the HpUreI hexamer and individual helices were readily apparent in the resulting low-resolution maps, but detail of the maps and phasing power was poor. To improve the phases, the program autoSHARP (Vonrhein, E. Blanc, P. Roversi, G. Bricogne, Meth. Mol. Biol. 364, 215 (2007)) was used. The figure of merit (FOM) from autoSHARP phasing was used to determine the high resolution cutoff for the experimental phases. A cutoff of 0.3 for the FOM of acentric reflections was used for this estimation, suggesting useful phases to about 4.7 Å resolution (Table 2).

TABLE 2
SHARP phasing statistics

| Dₘᵢₙ (□) | Dₘₐₓ (□) | FOM acentric | FOM centric |
|---|---|---|---|
| 38.99 | 16.33 | 0.89 | 0.65 |
| 16.33 | 12.09 | 0.92 | 0.71 |
| 12.09 | 10.03 | 0.89 | 0.65 |
| 10.03 | 8.76 | 0.83 | 0.54 |
| 8.76 | 7.88 | 0.82 | 0.54 |
| 7.88 | 7.21 | 0.75 | 0.50 |
| 7.21 | 6.70 | 0.70 | 0.46 |
| 6.70 | 6.28 | 0.57 | 0.32 |
| 6.28 | 5.92 | 0.50 | 0.30 |
| 5.92 | 5.63 | 0.46 | 0.27 |
| 5.63 | 5.37 | 0.37 | 0.22 |
| 5.37 | 5.15 | 0.40 | 0.22 |
| 5.15 | 4.95 | 0.34 | 0.15 |
| 4.95 | 4.77 | 0.31 | 0.15 |
| 4.77 | 4.61 | 0.25 | 0.17 |
| 4.61 | 4.47 | 0.21 | 0.11 |
| 4.47 | 4.33 | 0.18 | 0.10 |
| 4.33 | 4.21 | 0.16 | 0.08 |
| 4.21 | 4.10 | 0.14 | 0.07 |
| 4.10 | 4.00 | 0.11 | 0.07 |

### EXAMPLE 9

### Phase Improvement and Extension Through Solvent Flattening, Non-Crystallographic Symmetry, and Multicrystal Averaging

In order to improve and extend the initial experimental MAD phases, solvent flattening, 3-fold non-crystallographic symmetry (NCS) and multi-crystal averaging of data from crystals with different c-axis lengths were carried out. To obtain initial matrices for NCS averaging, 18 ideal helices were modeled into the experimental electron density representing three molecules in the asymmetric unit. Subsequently the NCS matrices from molecule A to molecule B and from molecule A to molecule C were obtained by the SSM superpose function of the program COOT (P. Emsley et al., W. G. Scott, K. Cowtan, Acta Cryst. D66, 486 (2010). A mask was placed around the electron density of molecule A and 3-fold NCS averaging and solvent flattening (68.9% solvent) were performed with the program DM, while NCS matrices were refined. Phases were extended from 4.5 to 3.5 Å in small resolution increments with the programs DM and DMMulti (K. D. Cowtan, Acta Cryst. D55, 1555 (1999)). At this point the right-handed twist of the helices and density for some of the larger side chains could be seen.

### EXAMPLE 10

### Model Building and Refinement

Density representing each of the six transmembrane helices of HpUreI protein was cut out using the program PHENIX (P. D. Adams et al., Acta Cryst. D66, 213 (2010)). An ideal helix was built into this density, slightly curved if required, using the sequence representing each helix. Helix 6 contained a pi-bulge and was built by hand using the program COOT. Cytoplasmic loops 1 and 2 (CL1 and CL2) and periplasmic loop 2 (PL2) were initially modeled into density using the program RAPPER (N. Furnham et al., Structure 14, 1313 (2006)). In places where the correct sequence could not be built initially, alanine was used temporarily. In order to generate the other two molecules of the asymmetric unit, the previously identified NCS matrices were used followed by rigid body refinement with the program PHENIX. The model was refined iteratively by cycles of manual adjustments using the program COOT and refinement with the program PHENIX, using TLS refinement with NCS and secondary structure restraints.

Upon analyzing the data with the UCLA anisotropy server (M. Strong, Proc. Natl. Acad. Sci. USA 103, 8060 (2006)), the data were found to be severely anisotropic with an effective resolution of 3.1 Å in the a* and b* directions, but only 3.5 Å in the c* direction. The data were ellipsoidally truncated and rescaled to minimize inclusion of poor diffraction data. The model was refined with the newly truncated data using jelly body refinement with the program REFMAC (M. D. Winn et al., Meth. Enz. 374, 300 (2003)), leading to significantly improved electron density maps which allowed further model improvement. Because of disorder, no model was built for the majority of periplasmic loop 1 (PL1, residues 59 to 73), which contains the engineered 6His insertion.

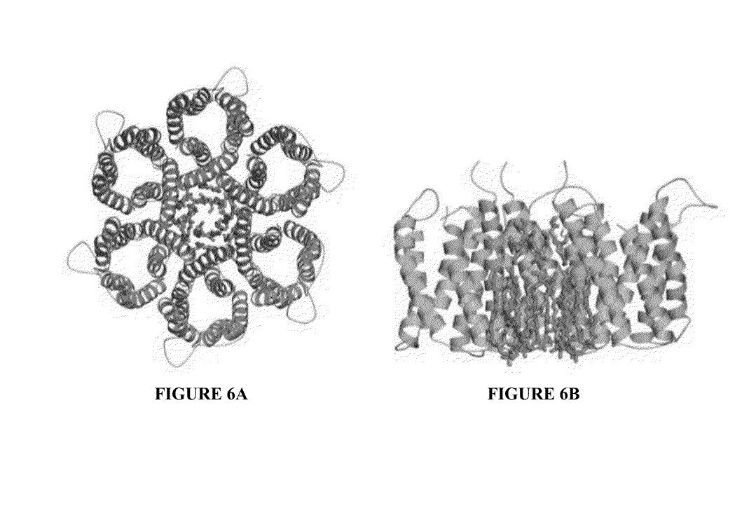

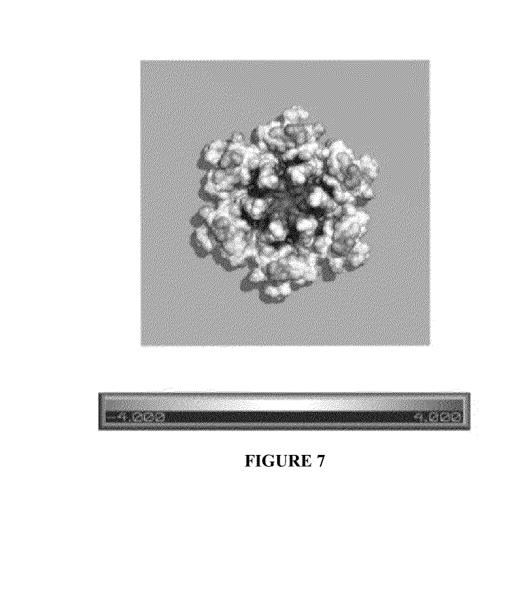

A final round of refinement was carried out with the program REFMAC against the non-truncated data with two TLS groups in each HpUreI protomer (residues 1-146 and 147-195 (FIGS. 6 and 7)), tight NCS restraints, and a jelly body value of 0.01. The final structure was refined to 3.26 Å with few Ramachandran outliers, small deviations from ideal geometry and predominantly preferred side chain rotamers (Table 3).

TABLE 3
Refinement statistics

| | |
|---|---|
| Resolution range (□) | 92.8-3.26 |
| No. of reflections | 17,190 |
| Working set | 16,318 |
| Test set | 872 |
| R_{work} (%) | 19.6 |
| R_{free} (%) | 27.8 |
| Rmsd bond lengths (□) | 0.011 |
| Rmsd bond angles (□) | 1.57 |
| Ramachandran analysis | |
| Allowed regions (%) | 98.7 |
| Disallowed regions (%) | 1.3 |

### EXAMPLE 11

### SeMet Anomalous Difference Maps

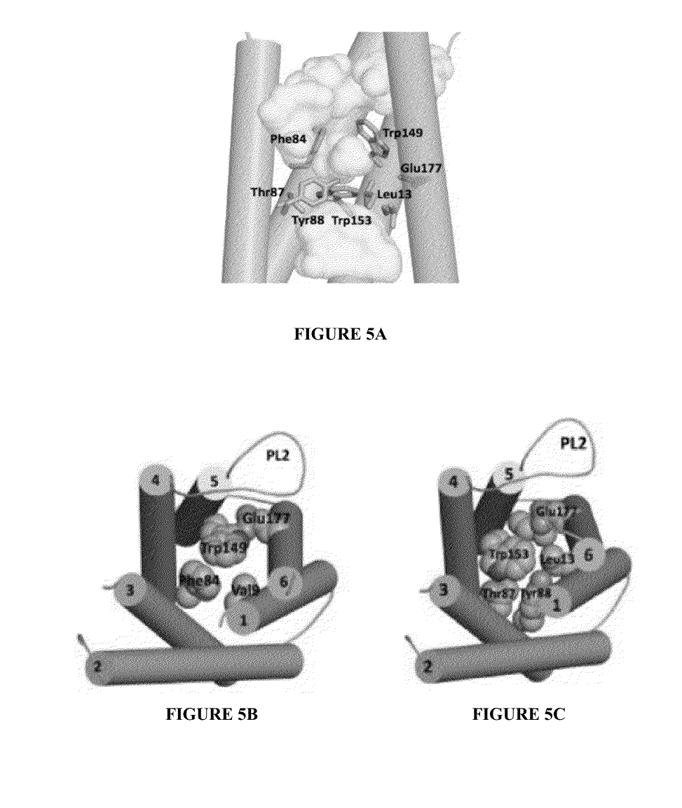

To validate the sequence assignment of the HpUreI crystal structure, additional methionine residues were engineered into various sites in the HpUreI sequence and labeled with SeMet. These proteins were crystallized and their anomalous differences Fourier maps inspected. For all five engineered sites, the location of the anomalous difference peak was less than 1 Å from the site of the methionine sulfur atom of the final model. The sites were Ile14, Ala148, Thr155, Leu173 and Ile191, in addition to the endogenous sites Met1, Met14 and Met127 (FIGS. 6 and 7), the latter one being situated in periplasmic loop 2 (PL2) (FIGS. 5A and 5B).

### EXAMPLE 12

### Crystal Structure of the H. Pylori Urea Channel, HpUreI

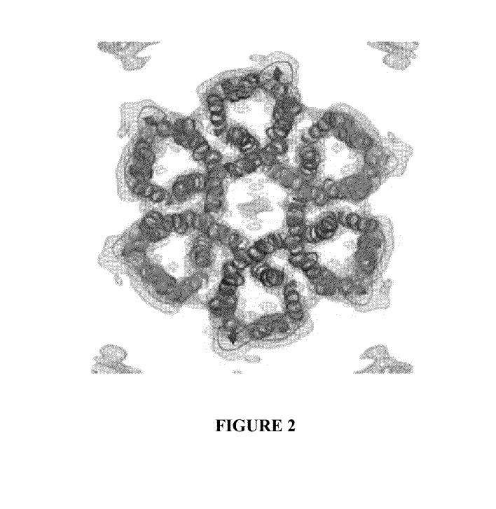

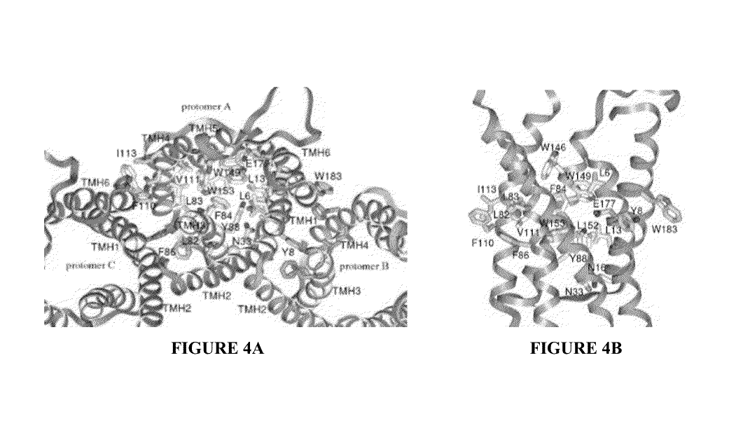

The HpUreI protein crystal structure reveals an arrangement of six protomers that form a compact hexameric ring (FIG. 2) about 95 Å in diameter and 45 Å in height. The center of the hexamer is filled with a lipid plug that forms a bilayer with electron density for six lipid tails in the periplasmic leaflet and for 18 tails in the cytoplasmic leaflet (FIGS. 6A, 6B, and 9). Each protomer of the HpUreI hexamer is able to function as a urea channel, as demonstrated by the lack of negative dominance in studies where coinjection of cRNA encoding an inactive His123Arg mutant into oocytes up to a three-fold excess compared to wild type did not reduce urea transport. The main contacts between protomers are between TMH1 and TMH2 of one protomer and TMH3 and TMH4 of a neighboring protomer (FIG. 4A).

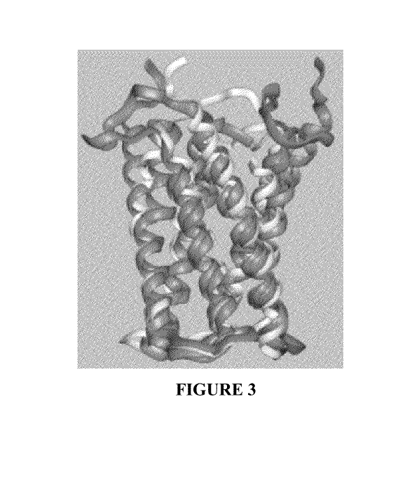

Each HpUreI protomer is a twisted bundle comprised of six slightly tilted transmembrane helices whose inward-facing side chains define a central channel pore (FIGS. 3, 4A, and 4B). The HpUreI protomer exhibits three-fold pseudosymmetry as evidenced by the high structural similarity of the backbone after 120° and 240° rotations around an axis through the center of the channel (FIG. 3). The repeating motif is a helical hairpin composed of a pair of antiparallel TMHs connected by a short cytoplasmic loop. However, closer helix-helix packing is observed between helices from adjacent repeated motifs: THM2 with THMH3, TMH4 with TMH5, and TMH1 with TMH6 (FIG. 5B).

Neither the N-terminus nor the C-terminus of an HpUreI protomer are readily accessible from the solvent. When viewed from the periplasm, the helices form a clockwise bundle connected by three short cytoplasmic loops (CL) and two longer periplasmic loops (PL) (FIG. 3A).

TMH1 contains three of the eleven residues that are absolutely conserved in the AmiS/Urel superfamily (see, Strugatsky, et al. Nature: volume 493, pages 255-258 (2013) doi:10.1038/nature11684, hereby incorporated by reference in its entirety), all on the helix face that is oriented inwards where it defines one side of the channel pore (FIG. 4A).

The longest helix, TMH2, is set back from the channel pore and nearest the lipid-filled center of the hexamer, contains no conserved residues, and does not contribute to the urea permeation pathway. Upon reaching the periplasmic side, TMH2 continues for several helical turns until the disordered section of PL1. At this point TMH2 reaches a height above the bilayer that is similar to the height of PL2 (FIG. 4A).

TMH3 is situated inwards of TMH2 and is a major contributor to the channel pore with four conserved residues, all in the pore, including Phe84 and Tyr88, which form parts of two constriction sites in the channel (FIG. 5A). TMH3 contacts TMH2 of the same protomer as well as the cytoplasmic half of TMH2 from a neighboring protomer (FIG. 4A).

TMH4 hardly contacts TMH3 and instead is in close contact with TMH5. TMH4 together with PL2, TMH5, CL3 and TMH6 constitutes the outer, bilayer-facing edge of the HpUreI hexamer (FIGS. 4A, 5A, and 5B).

TMH5 contains five tryptophans, three of which are highly conserved: Trp146, Trp149 and Trp153 (see, Strugatsky, et al. Nature: volume 493, pages 255-258 (2013) doi:10.1038/nature11684). Four of these, including the conserved ones, line the urea path, with the aromatic side chains of Trp149 and Trp153 being a major component of the two constriction sites (FIGS. 4A, 5A, 5B, and 5C).

In the middle of the membrane, Glul 77 of TMH6 is predicted to be protonated at pH 6.1. This residue is located just to the side of the channel pore and in position to hydrogen bond to urea. (FIGS. 5A, 5B, and 5C).

Using the HpUreI crystal structure to survey for the pathway for urea through HpUreI, electrostatic potential calculations based on the crystal structure show negative regions around PL2, likely part of the pH sensor, and positive values toward the center of the hexamer (FIG. 7). Although not bound by any particular theory, the electrostatic potential is likely to preorient the polar urea as it enters the channel from the periplasmic side such that its NH₂ moieties point towards the outside of each protomer towards PL2 and its carbonyl oxygen points towards TMH2.

Urea entry from the periplasm is likely through an irregularly-shaped vestibule starting roughly at the height of the bilayer edge and defined by a set of largely hydrophobic side chains including Leu2, Tyr76, Trp142, and Trp146. Urea would then pass through two constriction sites on either side of Glu177. The constriction site on the periplasmic side of Glu177 is likely defined by the side chains of conserved Leu6, Val9, Phe84 and Trp149. Just to the cytoplasmic side of Glu177 is a second likely constriction site defined by the side chains of conserved Leu13, Thr87, Tyr88, Leu152, and Trp153 (FIGS. 3, 4A, 5A, 5B, and 5C).

Although HpUreI crystals used in x-ray diffraction experiments were formed at pH 5.3, likely in the open form, in the electron density maps, Trp153 has its aromatic plane oriented perpendicular to the channel pore axis, which could appear to block passage (FIG. 5C). However, its indole side chain lacks structural restraints and appears to be able to reorient about χ₂ to allow passage of urea. Other aromatic side chains in the channel are likely to exhibit similar flexibility, which could allow transient π-π stacking of the surfaces of these side chains with planar urea, while preventing a proton leak.

In the HpUreI crystal structure, PL2 is located to the side of the pore, suggesting that channel gating might be mediated by pH-dependent displacement of a loop. Alternatively, gating could affect the structure or dynamics of the constriction sites, possibly through TMH rotations, perhaps mediated by His193 protonation-deprotonation in the C terminal segment. And there is likely transmission of the pH-induced conformational change to a change of conformation of the third cytoplasmic loop between TMH5 and TMH6 which then directly or indirectly recruits cytoplasmic urease (FIGS. 3, 4A, 5A, 5B, 5C, and 6).

A lipid bilayer forms a plug in the center of the HpUreI hexamer with electron density for six lipid tails in the periplasmic leaflet and for 18 tails in the cytoplasmic leaflet. This plug is narrower on the periplasmic side (17 Å vs. 28 Å diameter), where six copies of TMH2 converge, than on the cytoplasmic side, where the plug is lined by six copies of TMH2 and TMH3 (FIGS. 6A and 6B).

TABLE 4
Atomic coordinates for HpUreI protomer

| | | | | | | | | | | | | |
|---|---|---|---|---|---|---|---|---|---|---|---|---|
| ATOM | 1 | N | MET | A | 1 | −10.169 | 37.593 | 3.983 | 1.00 | 189.55 | A | N |
| ATOM | 2 | CA | MET | A | 1 | −9.673 | 36.376 | 4.694 | 1.00 | 182.75 | A | C |
| ATOM | 3 | CB | MET | A | 1 | −10.139 | 35.118 | 3.976 | 1.00 | 148.30 | A | C |
| ATOM | 4 | CG | MET | A | 1 | −11.613 | 34.831 | 4.200 | 1.00 | 188.90 | A | C |
| ATOM | 5 | SD | MET | A | 1 | −12.315 | 33.807 | 2.898 | 1.00 | 224.25 | A | S |
| ATOM | 6 | CE | MET | A | 1 | −11.586 | 32.231 | 3.332 | 1.00 | 167.40 | A | C |
| ATOM | 7 | C | MET | A | 1 | −8.161 | 36.350 | 4.857 | 1.00 | 192.03 | A | C |
| ATOM | 8 | O | MET | A | 1 | −7.597 | 35.370 | 5.361 | 1.00 | 173.37 | A | O |
| ATOM | 9 | N | LEU | A | 2 | −7.519 | 37.448 | 4.460 | 1.00 | 191.46 | A | N |
| ATOM | 10 | CA | LEU | A | 2 | −6.065 | 37.579 | 4.511 | 1.00 | 168.46 | A | C |
| ATOM | 11 | CB | LEU | A | 2 | −5.588 | 38.784 | 3.688 | 1.00 | 171.29 | A | C |
| ATOM | 12 | CG | LEU | A | 2 | −5.883 | 38.752 | 2.177 | 1.00 | 224.57 | A | C |
| ATOM | 13 | CD1 | LEU | A | 2 | −7.210 | 39.448 | 1.852 | 1.00 | 233.13 | A | C |
| ATOM | 14 | CD2 | LEU | A | 2 | −4.731 | 39.326 | 1.357 | 1.00 | 145.04 | A | C |
| ATOM | 15 | C | LEU | A | 2 | −5.552 | 37.652 | 5.943 | 1.00 | 177.19 | A | C |
| ATOM | 16 | O | LEU | A | 2 | −4.423 | 37.249 | 6.209 | 1.00 | 170.43 | A | O |
| ATOM | 17 | N | GLY | A | 3 | −6.388 | 38.147 | 6.857 | 1.00 | 179.69 | A | N |
| ATOM | 18 | CA | GLY | A | 3 | −6.110 | 38.078 | 8.294 | 1.00 | 180.10 | A | C |
| ATOM | 19 | C | GLY | A | 3 | −5.860 | 36.639 | 8.701 | 1.00 | 175.78 | A | C |
| ATOM | 20 | O | GLY | A | 3 | −4.866 | 36.331 | 9.367 | 1.00 | 191.20 | A | O |
| ATOM | 21 | N | LEU | A | 4 | −6.774 | 35.764 | 8.287 | 1.00 | 159.08 | A | N |
| ATOM | 22 | CA | LEU | A | 4 | −6.633 | 34.337 | 8.474 | 1.00 | 150.77 | A | C |
| ATOM | 23 | CB | LEU | A | 4 | −7.934 | 33.642 | 8.120 | 1.00 | 142.87 | A | C |
| ATOM | 24 | CG | LEU | A | 4 | −8.607 | 32.914 | 9.267 | 1.00 | 164.66 | A | C |
| ATOM | 25 | CD1 | LEU | A | 4 | −9.867 | 32.274 | 8.731 | 1.00 | 204.17 | A | C |
| ATOM | 26 | CD2 | LEU | A | 4 | −7.691 | 31.849 | 9.851 | 1.00 | 162.21 | A | C |
| ATOM | 27 | C | LEU | A | 4 | −5.505 | 33.761 | 7.628 | 1.00 | 157.72 | A | C |
| ATOM | 28 | O | LEU | A | 4 | −4.492 | 33.297 | 8.157 | 1.00 | 156.44 | A | O |
| ATOM | 29 | N | VAL | A | 5 | −5.687 | 33.804 | 6.312 | 1.00 | 152.14 | A | N |
| ATOM | 30 | CA | VAL | A | 5 | −4.753 | 33.185 | 5.388 | 1.00 | 146.78 | A | C |
| ATOM | 31 | CB | VAL | A | 5 | −5.062 | 33.554 | 3.919 | 1.00 | 155.76 | A | C |
| ATOM | 32 | CG1 | VAL | A | 5 | −3.958 | 33.044 | 2.997 | 1.00 | 132.99 | A | C |
| ATOM | 33 | CG2 | VAL | A | 5 | −6.408 | 32.996 | 3.495 | 1.00 | 163.24 | A | C |
| ATOM | 34 | C | VAL | A | 5 | −3.323 | 33.584 | 5.698 | 1.00 | 159.44 | A | C |
| ATOM | 35 | O | VAL | A | 5 | −2.456 | 32.719 | 5.837 | 1.00 | 161.23 | A | O |
| ATOM | 36 | N | LEU | A | 6 | −3.089 | 34.890 | 5.820 | 1.00 | 161.39 | A | N |
| ATOM | 37 | CA | LEU | A | 6 | −1.731 | 35.408 | 5.961 | 1.00 | 167.97 | A | C |
| ATOM | 38 | CB | LEU | A | 6 | −1.634 | 36.873 | 5.521 | 1.00 | 164.96 | A | C |
| ATOM | 39 | CG | LEU | A | 6 | −1.697 | 37.118 | 4.011 | 1.00 | 147.62 | A | C |
| ATOM | 40 | CD1 | LEU | A | 6 | −1.440 | 38.565 | 3.699 | 1.00 | 140.44 | A | C |
| ATOM | 41 | CD2 | LEU | A | 6 | −0.670 | 36.278 | 3.273 | 1.00 | 177.85 | A | C |
| ATOM | 42 | C | LEU | A | 6 | −1.149 | 35.199 | 7.351 | 1.00 | 155.24 | A | C |
| ATOM | 43 | O | LEU | A | 6 | 0.070 | 35.136 | 7.510 | 1.00 | 150.59 | A | O |
| ATOM | 44 | N | LEU | A | 7 | −2.012 | 35.075 | 8.352 | 1.00 | 141.49 | A | N |
| ATOM | 45 | CA | LEU | A | 7 | −1.545 | 34.659 | 9.665 | 1.00 | 126.16 | A | C |
| ATOM | 46 | CB | LEU | A | 7 | −2.713 | 34.473 | 10.627 | 1.00 | 158.65 | A | C |
| ATOM | 47 | CG | LEU | A | 7 | −2.334 | 33.808 | 11.958 | 1.00 | 168.51 | A | C |
| ATOM | 48 | CD1 | LEU | A | 7 | −1.305 | 34.643 | 12.723 | 1.00 | 177.07 | A | C |
| ATOM | 49 | CD2 | LEU | A | 7 | −3.564 | 33.539 | 12.816 | 1.00 | 180.29 | A | C |
| ATOM | 50 | C | LEU | A | 7 | −0.742 | 33.356 | 9.578 | 1.00 | 140.00 | A | C |
| ATOM | 51 | O | LEU | A | 7 | 0.318 | 33.217 | 10.192 | 1.00 | 159.40 | A | O |
| ATOM | 52 | N | TYR | A | 8 | −1.245 | 32.407 | 8.796 | 1.00 | 130.91 | A | N |
| ATOM | 53 | CA | TYR | A | 8 | −0.626 | 31.087 | 8.684 | 1.00 | 129.70 | A | C |
| ATOM | 54 | CB | TYR | A | 8 | −1.695 | 30.027 | 8.435 | 1.00 | 145.26 | A | C |
| ATOM | 55 | CG | TYR | A | 8 | −2.581 | 29.820 | 9.638 | 1.00 | 147.56 | A | C |
| ATOM | 56 | CD1 | TYR | A | 8 | −2.213 | 28.929 | 10.652 | 1.00 | 134.69 | A | C |
| ATOM | 57 | CE1 | TYR | A | 8 | −3.007 | 28.744 | 11.772 | 1.00 | 146.15 | A | C |
| ATOM | 58 | CZ | TYR | A | 8 | −4.181 | 29.471 | 11.889 | 1.00 | 183.63 | A | C |
| ATOM | 59 | OH | TYR | A | 8 | −4.985 | 29.298 | 12.984 | 1.00 | 191.15 | A | O |
| ATOM | 60 | CE2 | TYR | A | 8 | −4.562 | 30.373 | 10.906 | 1.00 | 162.20 | A | C |
| ATOM | 61 | CD2 | TYR | A | 8 | −3.763 | 30.545 | 9.791 | 1.00 | 145.08 | A | C |
| ATOM | 62 | C | TYR | A | 8 | 0.484 | 31.032 | 7.641 | 1.00 | 146.38 | A | C |
| ATOM | 63 | O | TYR | A | 8 | 1.463 | 30.301 | 7.807 | 1.00 | 148.45 | A | O |
| ATOM | 64 | N | VAL | A | 9 | 0.338 | 31.832 | 6.586 | 1.00 | 137.16 | A | N |
| ATOM | 65 | CA | VAL | A | 9 | 1.437 | 32.083 | 5.656 | 1.00 | 138.42 | A | C |
| ATOM | 66 | CB | VAL | A | 9 | 1.066 | 33.118 | 4.570 | 1.00 | 170.54 | A | C |
| ATOM | 67 | CG1 | VAL | A | 9 | 2.298 | 33.587 | 3.809 | 1.00 | 170.41 | A | C |
| ATOM | 68 | CG2 | VAL | A | 9 | 0.072 | 32.533 | 3.589 | 1.00 | 181.87 | A | C |
| ATOM | 69 | C | VAL | A | 9 | 2.648 | 32.567 | 6.442 | 1.00 | 150.13 | A | C |
| ATOM | 70 | O | VAL | A | 9 | 3.786 | 32.355 | 6.035 | 1.00 | 150.07 | A | O |
| ATOM | 71 | N | GLY | A | 10 | 2.386 | 33.207 | 7.579 | 1.00 | 140.61 | A | N |
| ATOM | 72 | CA | GLY | A | 10 | 3.442 | 33.591 | 8.500 | 1.00 | 156.72 | A | C |
| ATOM | 73 | C | GLY | A | 10 | 4.301 | 32.421 | 8.942 | 1.00 | 150.49 | A | C |
| ATOM | 74 | O | GLY | A | 10 | 5.503 | 32.367 | 8.647 | 1.00 | 156.48 | A | O |
| ATOM | 75 | N | ILE | A | 11 | 3.683 | 31.485 | 9.652 | 1.00 | 146.22 | A | N |
| ATOM | 76 | CA | ILE | A | 11 | 4.413 | 30.352 | 10.219 | 1.00 | 149.41 | A | C |
| ATOM | 77 | CB | ILE | A | 11 | 3.527 | 29.471 | 11.108 | 1.00 | 148.98 | A | C |
| ATOM | 78 | CG1 | ILE | A | 11 | 2.429 | 30.322 | 11.720 | 1.00 | 166.54 | A | C |
| ATOM | 79 | CD1 | ILE | A | 11 | 1.119 | 29.610 | 11.913 | 1.00 | 185.28 | A | C |
| ATOM | 80 | CG2 | ILE | A | 11 | 4.359 | 28.802 | 12.196 | 1.00 | 118.06 | A | C |
| ATOM | 81 | C | ILE | A | 11 | 5.003 | 29.493 | 9.125 | 1.00 | 140.67 | A | C |
| ATOM | 82 | O | ILE | A | 11 | 6.183 | 29.173 | 9.175 | 1.00 | 139.38 | A | O |
| ATOM | 83 | N | VAL | A | 12 | 4.188 | 29.136 | 8.135 | 1.00 | 142.85 | A | N |
| ATOM | 84 | CA | VAL | A | 12 | 4.635 | 28.262 | 7.057 | 1.00 | 135.78 | A | C |
| ATOM | 85 | CB | VAL | A | 12 | 3.529 | 27.994 | 6.031 | 1.00 | 129.76 | A | C |
| ATOM | 86 | CG1 | VAL | A | 12 | 3.207 | 29.244 | 5.239 | 1.00 | 132.88 | A | C |
| ATOM | 87 | CG2 | VAL | A | 12 | 3.949 | 26.878 | 5.087 | 1.00 | 155.07 | A | C |
| ATOM | 88 | C | VAL | A | 12 | 5.857 | 28.822 | 6.344 | 1.00 | 146.03 | A | C |
| ATOM | 89 | O | VAL | A | 12 | 6.734 | 28.058 | 5.926 | 1.00 | 154.87 | A | O |
| ATOM | 90 | N | LEU | A | 13 | 5.905 | 30.147 | 6.201 | 1.00 | 148.03 | A | N |
| ATOM | 91 | CA | LEU | A | 13 | 7.070 | 30.801 | 5.622 | 1.00 | 148.35 | A | C |
| ATOM | 92 | CB | LEU | A | 13 | 6.770 | 32.232 | 5.187 | 1.00 | 156.85 | A | C |
| ATOM | 93 | CG | LEU | A | 13 | 6.565 | 32.379 | 3.679 | 1.00 | 154.07 | A | C |
| ATOM | 94 | CD1 | LEU | A | 13 | 6.221 | 33.815 | 3.340 | 1.00 | 143.78 | A | C |
| ATOM | 95 | CD2 | LEU | A | 13 | 7.788 | 31.901 | 2.893 | 1.00 | 165.11 | A | C |
| ATOM | 96 | C | LEU | A | 13 | 8.251 | 30.776 | 6.566 | 1.00 | 148.81 | A | C |
| ATOM | 97 | O | LEU | A | 13 | 9.372 | 30.507 | 6.146 | 1.00 | 169.97 | A | O |
| ATOM | 98 | N | ILE | A | 14 | 7.997 | 31.054 | 7.840 | 1.00 | 155.02 | A | N |
| ATOM | 99 | CA | ILE | A | 14 | 9.009 | 30.882 | 8.880 | 1.00 | 167.87 | A | C |
| ATOM | 100 | CB | ILE | A | 14 | 8.489 | 31.335 | 10.262 | 1.00 | 157.37 | A | C |
| ATOM | 101 | CG1 | ILE | A | 14 | 8.622 | 32.842 | 10.406 | 1.00 | 174.79 | A | C |
| ATOM | 102 | CD1 | ILE | A | 14 | 8.076 | 33.382 | 11.708 | 1.00 | 182.61 | A | C |
| ATOM | 103 | CG2 | ILE | A | 14 | 9.259 | 30.665 | 11.386 | 1.00 | 141.08 | A | C |
| ATOM | 104 | C | ILE | A | 14 | 9.467 | 29.424 | 8.930 | 1.00 | 163.34 | A | C |
| ATOM | 105 | O | ILE | A | 14 | 10.665 | 29.153 | 8.996 | 1.00 | 180.25 | A | O |
| ATOM | 106 | N | SER | A | 15 | 8.510 | 28.495 | 8.881 | 1.00 | 151.68 | A | N |
| ATOM | 107 | CA | SER | A | 15 | 8.810 | 27.066 | 8.965 | 1.00 | 150.36 | A | C |
| ATOM | 108 | CB | SER | A | 15 | 7.537 | 26.231 | 8.807 | 1.00 | 157.43 | A | C |
| ATOM | 109 | OG | SER | A | 15 | 7.833 | 24.870 | 8.523 | 1.00 | 191.98 | A | O |
| ATOM | 110 | C | SER | A | 15 | 9.844 | 26.666 | 7.924 | 1.00 | 164.19 | A | C |
| ATOM | 111 | O | SER | A | 15 | 10.886 | 26.107 | 8.264 | 1.00 | 178.70 | A | O |
| ATOM | 112 | N | ASN | A | 16 | 9.558 | 26.968 | 6.660 | 1.00 | 160.47 | A | N |
| ATOM | 113 | CA | ASN | A | 16 | 10.464 | 26.642 | 5.561 | 1.00 | 170.93 | A | C |
| ATOM | 114 | CB | ASN | A | 16 | 9.848 | 27.088 | 4.240 | 1.00 | 140.88 | A | C |
| ATOM | 115 | CG | ASN | A | 16 | 8.788 | 26.122 | 3.741 | 1.00 | 160.78 | A | C |
| ATOM | 116 | OD1 | ASN | A | 16 | 8.896 | 24.891 | 3.891 | 1.00 | 194.12 | A | O |
| ATOM | 117 | ND2 | ASN | A | 16 | 7.744 | 26.682 | 3.137 | 1.00 | 166.48 | A | N |
| ATOM | 118 | C | ASN | A | 16 | 11.867 | 27.222 | 5.717 | 1.00 | 177.88 | A | C |
| ATOM | 119 | O | ASN | A | 16 | 12.864 | 26.573 | 5.361 | 1.00 | 175.51 | A | O |
| ATOM | 120 | N | GLY | A | 17 | 11.936 | 28.441 | 6.257 | 1.00 | 179.94 | A | N |
| ATOM | 121 | CA | GLY | A | 17 | 13.209 | 29.066 | 6.603 | 1.00 | 197.24 | A | C |
| ATOM | 122 | C | GLY | A | 17 | 14.022 | 28.247 | 7.597 | 1.00 | 197.96 | A | C |
| ATOM | 123 | O | GLY | A | 17 | 15.157 | 27.834 | 7.305 | 1.00 | 208.34 | A | O |
| ATOM | 124 | N | ILE | A | 18 | 13.439 | 28.004 | 8.770 | 1.00 | 147.11 | A | N |
| ATOM | 125 | CA | ILE | A | 18 | 14.101 | 27.224 | 9.808 | 1.00 | 163.48 | A | C |
| ATOM | 126 | CB | ILE | A | 18 | 13.215 | 27.095 | 11.054 | 1.00 | 176.57 | A | C |
| ATOM | 127 | CG1 | ILE | A | 18 | 12.949 | 28.490 | 11.634 | 1.00 | 165.53 | A | C |
| ATOM | 128 | CD1 | ILE | A | 18 | 11.817 | 28.526 | 12.639 | 1.00 | 189.92 | A | C |
| ATOM | 129 | CG2 | ILE | A | 18 | 13.875 | 26.211 | 12.102 | 1.00 | 174.10 | A | C |
| ATOM | 130 | C | ILE | A | 18 | 14.539 | 25.849 | 9.312 | 1.00 | 178.28 | A | C |
| ATOM | 131 | O | ILE | A | 18 | 15.631 | 25.393 | 9.641 | 1.00 | 177.82 | A | O |
| ATOM | 132 | N | CYS | A | 19 | 13.697 | 25.199 | 8.516 | 1.00 | 173.93 | A | N |
| ATOM | 133 | CA | CYS | A | 19 | 13.959 | 23.838 | 8.058 | 1.00 | 159.55 | A | C |
| ATOM | 134 | CB | CYS | A | 19 | 12.693 | 23.203 | 7.501 | 1.00 | 169.39 | A | C |
| ATOM | 135 | SG | CYS | A | 19 | 11.452 | 22.880 | 8.776 | 1.00 | 223.58 | A | S |
| ATOM | 136 | C | CYS | A | 19 | 15.079 | 23.748 | 7.037 | 1.00 | 154.66 | A | C |
| ATOM | 137 | O | CYS | A | 19 | 15.576 | 22.662 | 6.780 | 1.00 | 181.87 | A | O |
| ATOM | 138 | N | GLY | A | 20 | 15.459 | 24.876 | 6.443 | 1.00 | 151.64 | A | N |
| ATOM | 139 | CA | GLY | A | 20 | 16.650 | 24.932 | 5.593 | 1.00 | 181.42 | A | C |
| ATOM | 140 | C | GLY | A | 20 | 17.882 | 25.174 | 6.450 | 1.00 | 191.01 | A | C |
| ATOM | 141 | O | GLY | A | 20 | 19.027 | 24.959 | 6.001 | 1.00 | 223.22 | A | O |
| ATOM | 142 | N | LEU | A | 21 | 17.636 | 25.596 | 7.695 | 1.00 | 194.97 | A | N |
| ATOM | 143 | CA | LEU | A | 21 | 18.694 | 25.934 | 8.642 | 1.00 | 184.13 | A | C |
| ATOM | 144 | CB | LEU | A | 21 | 18.325 | 27.159 | 9.476 | 1.00 | 185.32 | A | C |
| ATOM | 145 | CG | LEU | A | 21 | 18.004 | 28.444 | 8.731 | 1.00 | 188.59 | A | C |
| ATOM | 146 | CD1 | LEU | A | 21 | 17.495 | 29.471 | 9.725 | 1.00 | 189.99 | A | C |
| ATOM | 147 | CD2 | LEU | A | 21 | 19.235 | 28.959 | 8.013 | 1.00 | 176.52 | A | C |
| ATOM | 148 | C | LEU | A | 21 | 18.998 | 24.767 | 9.549 | 1.00 | 162.91 | A | C |
| ATOM | 149 | O | LEU | A | 21 | 20.076 | 24.201 | 9.437 | 1.00 | 178.76 | A | O |
| ATOM | 150 | N | THR | A | 22 | 18.059 | 24.398 | 10.426 | 1.00 | 162.64 | A | N |
| ATOM | 151 | CA | THR | A | 22 | 18.232 | 23.201 | 11.269 | 1.00 | 186.37 | A | C |
| ATOM | 152 | CB | THR | A | 22 | 17.187 | 23.092 | 12.423 | 1.00 | 173.77 | A | C |
| ATOM | 153 | OG1 | THR | A | 22 | 15.875 | 23.453 | 11.961 | 1.00 | 164.05 | A | O |
| ATOM | 154 | CG2 | THR | A | 22 | 17.566 | 23.991 | 13.586 | 1.00 | 219.25 | A | C |
| ATOM | 155 | C | THR | A | 22 | 18.285 | 21.908 | 10.425 | 1.00 | 172.27 | A | C |
| ATOM | 156 | O | THR | A | 22 | 18.829 | 20.880 | 10.867 | 1.00 | 154.09 | A | O |
| ATOM | 157 | N | LYS | A | 23 | 17.735 | 21.996 | 9.208 | 1.00 | 171.28 | A | N |
| ATOM | 158 | CA | LYS | A | 23 | 17.718 | 20.932 | 8.172 | 1.00 | 159.90 | A | C |
| ATOM | 159 | CB | LYS | A | 23 | 19.040 | 20.809 | 7.434 | 1.00 | 183.46 | A | C |
| ATOM | 160 | CG | LYS | A | 23 | 19.077 | 21.803 | 6.278 | 1.00 | 164.70 | A | C |
| ATOM | 161 | CD | LYS | A | 23 | 20.350 | 21.672 | 5.476 | 1.00 | 171.55 | A | C |
| ATOM | 162 | CE | LYS | A | 23 | 21.414 | 22.665 | 5.960 | 1.00 | 205.55 | A | C |
| ATOM | 163 | NZ | LYS | A | 23 | 22.401 | 22.877 | 4.871 | 1.00 | 254.21 | A | N |
| ATOM | 164 | C | LYS | A | 23 | 17.095 | 19.598 | 8.505 | 1.00 | 167.44 | A | C |
| ATOM | 165 | O | LYS | A | 23 | 17.771 | 18.582 | 8.764 | 1.00 | 164.27 | A | O |
| ATOM | 166 | N | VAL | A | 24 | 15.775 | 19.616 | 8.426 | 1.00 | 147.89 | A | N |
| ATOM | 167 | CA | VAL | A | 24 | 14.999 | 18.524 | 8.935 | 1.00 | 162.46 | A | C |
| ATOM | 168 | CB | VAL | A | 24 | 13.980 | 19.019 | 9.998 | 1.00 | 146.65 | A | C |
| ATOM | 169 | CG1 | VAL | A | 24 | 13.134 | 20.129 | 9.468 | 1.00 | 126.47 | A | C |
| ATOM | 170 | CG2 | VAL | A | 24 | 13.099 | 17.885 | 10.498 | 1.00 | 186.26 | A | C |
| ATOM | 171 | C | VAL | A | 24 | 14.358 | 17.707 | 7.814 | 1.00 | 157.17 | A | C |
| ATOM | 172 | O | VAL | A | 24 | 13.945 | 18.248 | 6.775 | 1.00 | 170.62 | A | O |
| ATOM | 173 | N | ASP | A | 25 | 14.426 | 16.391 | 8.016 | 1.00 | 131.26 | A | N |
| ATOM | 174 | CA | ASP | A | 25 | 13.472 | 15.415 | 7.538 | 1.00 | 137.79 | A | C |
| ATOM | 175 | CB | ASP | A | 25 | 12.887 | 14.769 | 8.797 | 1.00 | 149.37 | A | C |
| ATOM | 176 | CG | ASP | A | 25 | 11.819 | 13.745 | 8.507 | 1.00 | 156.03 | A | C |
| ATOM | 177 | OD1 | ASP | A | 25 | 12.157 | 12.634 | 8.026 | 1.00 | 167.03 | A | O |
| ATOM | 178 | OD2 | ASP | A | 25 | 10.642 | 14.048 | 8.803 | 1.00 | 147.77 | A | O |
| ATOM | 179 | C | ASP | A | 25 | 12.363 | 16.006 | 6.640 | 1.00 | 150.16 | A | C |
| ATOM | 180 | O | ASP | A | 25 | 11.339 | 16.468 | 7.142 | 1.00 | 148.84 | A | O |
| ATOM | 181 | N | PRO | A | 26 | 12.555 | 15.964 | 5.306 | 1.00 | 114.11 | A | N |
| ATOM | 182 | CA | PRO | A | 26 | 11.660 | 16.628 | 4.370 | 1.00 | 133.86 | A | C |
| ATOM | 183 | CB | PRO | A | 26 | 12.134 | 16.121 | 3.006 | 1.00 | 130.45 | A | C |
| ATOM | 184 | CG | PRO | A | 26 | 13.545 | 15.732 | 3.230 | 1.00 | 124.90 | A | C |
| ATOM | 185 | CD | PRO | A | 26 | 13.538 | 15.130 | 4.601 | 1.00 | 120.32 | A | C |
| ATOM | 186 | C | PRO | A | 26 | 10.199 | 16.250 | 4.578 | 1.00 | 131.84 | A | C |
| ATOM | 187 | O | PRO | A | 26 | 9.312 | 17.049 | 4.251 | 1.00 | 143.98 | A | O |
| ATOM | 188 | N | LYS | A | 27 | 9.947 | 15.055 | 5.112 | 1.00 | 109.12 | A | N |
| ATOM | 189 | CA | LYS | A | 27 | 8.587 | 14.661 | 5.433 | 1.00 | 109.86 | A | C |
| ATOM | 190 | CB | LYS | A | 27 | 8.532 | 13.232 | 5.970 | 1.00 | 97.08 | A | C |
| ATOM | 191 | CG | LYS | A | 27 | 9.185 | 12.147 | 5.129 | 1.00 | 115.05 | A | C |
| ATOM | 192 | CD | LYS | A | 27 | 8.180 | 11.512 | 4.175 | 1.00 | 140.34 | A | C |
| ATOM | 193 | CE | LYS | A | 27 | 8.642 | 10.158 | 3.634 | 1.00 | 196.55 | A | C |
| ATOM | 194 | NZ | LYS | A | 27 | 7.691 | 9.516 | 2.662 | 1.00 | 178.81 | A | N |
| ATOM | 195 | C | LYS | A | 27 | 7.979 | 15.606 | 6.474 | 1.00 | 122.18 | A | C |
| ATOM | 196 | O | LYS | A | 27 | 6.810 | 15.959 | 6.388 | 1.00 | 139.18 | A | O |
| ATOM | 197 | N | SER | A | 28 | 8.768 | 16.011 | 7.465 | 1.00 | 130.86 | A | N |
| ATOM | 198 | CA | SER | A | 28 | 8.239 | 16.802 | 8.582 | 1.00 | 143.12 | A | C |
| ATOM | 199 | CB | SER | A | 28 | 9.212 | 16.816 | 9.756 | 1.00 | 137.44 | A | C |
| ATOM | 200 | OG | SER | A | 28 | 9.563 | 15.501 | 10.133 | 1.00 | 114.04 | A | O |
| ATOM | 201 | C | SER | A | 28 | 7.925 | 18.225 | 8.174 | 1.00 | 130.00 | A | C |
| ATOM | 202 | O | SER | A | 28 | 6.861 | 18.753 | 8.502 | 1.00 | 140.61 | A | O |
| ATOM | 203 | N | THR | A | 29 | 8.856 | 18.844 | 7.458 | 1.00 | 126.10 | A | N |
| ATOM | 204 | CA | THR | A | 29 | 8.627 | 20.183 | 6.950 | 1.00 | 134.92 | A | C |
| ATOM | 205 | CB | THR | A | 29 | 9.751 | 20.638 | 5.995 | 1.00 | 133.90 | A | C |
| ATOM | 206 | OG1 | THR | A | 29 | 9.569 | 20.043 | 4.714 | 1.00 | 128.38 | A | O |
| ATOM | 207 | CG2 | THR | A | 29 | 11.087 | 20.228 | 6.499 | 1.00 | 134.19 | A | C |
| ATOM | 208 | C | THR | A | 29 | 7.291 | 20.254 | 6.205 | 1.00 | 132.98 | A | C |
| ATOM | 209 | O | THR | A | 29 | 6.512 | 21.199 | 6.386 | 1.00 | 146.23 | A | O |
| ATOM | 210 | N | ALA | A | 30 | 7.033 | 19.232 | 5.388 | 1.00 | 116.20 | A | N |
| ATOM | 211 | CA | ALA | A | 30 | 5.927 | 19.259 | 4.444 | 1.00 | 125.18 | A | C |
| ATOM | 212 | CB | ALA | A | 30 | 5.939 | 18.010 | 3.572 | 1.00 | 116.54 | A | C |
| ATOM | 213 | C | ALA | A | 30 | 4.601 | 19.407 | 5.157 | 1.00 | 111.65 | A | C |
| ATOM | 214 | O | ALA | A | 30 | 3.708 | 20.110 | 4.692 | 1.00 | 105.47 | A | O |
| ATOM | 215 | N | VAL | A | 31 | 4.487 | 18.750 | 6.301 | 1.00 | 110.30 | A | N |
| ATOM | 216 | CA | VAL | A | 31 | 3.277 | 18.801 | 7.097 | 1.00 | 108.68 | A | C |
| ATOM | 217 | CB | VAL | A | 31 | 3.546 | 18.407 | 8.558 | 1.00 | 121.92 | A | C |
| ATOM | 218 | CG1 | VAL | A | 31 | 2.276 | 18.493 | 9.390 | 1.00 | 111.49 | A | C |
| ATOM | 219 | CG2 | VAL | A | 31 | 4.106 | 17.002 | 8.625 | 1.00 | 140.12 | A | C |
| ATOM | 220 | C | VAL | A | 31 | 2.710 | 20.201 | 7.090 | 1.00 | 121.06 | A | C |
| ATOM | 221 | O | VAL | A | 31 | 1.559 | 20.410 | 6.704 | 1.00 | 122.45 | A | O |
| ATOM | 222 | N | MET | A | 32 | 3.526 | 21.156 | 7.529 | 1.00 | 124.57 | A | N |
| ATOM | 223 | CA | MET | A | 32 | 3.083 | 22.525 | 7.653 | 1.00 | 128.61 | A | C |
| ATOM | 224 | CB | MET | A | 32 | 4.186 | 23.397 | 8.249 | 1.00 | 150.05 | A | C |
| ATOM | 225 | CG | MET | A | 32 | 3.732 | 24.819 | 8.525 | 1.00 | 151.11 | A | C |
| ATOM | 226 | SD | MET | A | 32 | 2.039 | 24.920 | 9.171 | 1.00 | 179.70 | A | S |
| ATOM | 227 | CE | MET | A | 32 | 2.276 | 24.579 | 10.912 | 1.00 | 144.75 | A | C |
| ATOM | 228 | C | MET | A | 32 | 2.612 | 23.033 | 6.298 | 1.00 | 119.57 | A | C |
| ATOM | 229 | O | MET | A | 32 | 1.479 | 23.497 | 6.161 | 1.00 | 128.38 | A | O |
| ATOM | 230 | N | ASN | A | 33 | 3.461 | 22.879 | 5.290 | 1.00 | 112.53 | A | N |
| ATOM | 231 | CA | ASN | A | 33 | 3.127 | 23.294 | 3.935 | 1.00 | 114.15 | A | C |
| ATOM | 232 | CB | ASN | A | 33 | 4.245 | 22.894 | 2.998 | 1.00 | 108.65 | A | C |
| ATOM | 233 | CG | ASN | A | 33 | 5.544 | 23.574 | 3.339 | 1.00 | 125.34 | A | C |
| ATOM | 234 | OD1 | ASN | A | 33 | 5.754 | 24.729 | 2.979 | 1.00 | 141.10 | A | O |
| ATOM | 235 | ND2 | ASN | A | 33 | 6.425 | 22.867 | 4.038 | 1.00 | 123.66 | A | N |
| ATOM | 236 | C | ASN | A | 33 | 1.797 | 22.731 | 3.447 | 1.00 | 123.37 | A | C |
| ATOM | 237 | O | ASN | A | 33 | 1.097 | 23.375 | 2.679 | 1.00 | 124.29 | A | O |
| ATOM | 238 | N | PHE | A | 34 | 1.455 | 21.530 | 3.909 | 1.00 | 124.07 | A | N |
| ATOM | 239 | CA | PHE | A | 34 | 0.161 | 20.933 | 3.607 | 1.00 | 119.05 | A | C |
| ATOM | 240 | CB | PHE | A | 34 | 0.098 | 19.490 | 4.081 | 1.00 | 123.00 | A | C |
| ATOM | 241 | CG | PHE | A | 34 | 0.591 | 18.499 | 3.080 | 1.00 | 118.43 | A | C |
| ATOM | 242 | CD1 | PHE | A | 34 | −0.114 | 18.269 | 1.920 | 1.00 | 93.58 | A | C |
| ATOM | 243 | CE1 | PHE | A | 34 | 0.349 | 17.346 | 1.015 | 1.00 | 100.64 | A | C |
| ATOM | 244 | CZ | PHE | A | 34 | 1.519 | 16.643 | 1.259 | 1.00 | 100.41 | A | C |
| ATOM | 245 | CE2 | PHE | A | 34 | 2.231 | 16.854 | 2.412 | 1.00 | 96.27 | A | C |
| ATOM | 246 | CD2 | PHE | A | 34 | 1.764 | 17.776 | 3.320 | 1.00 | 120.73 | A | C |
| ATOM | 247 | C | PHE | A | 34 | −0.970 | 21.692 | 4.261 | 1.00 | 120.69 | A | C |
| ATOM | 248 | O | PHE | A | 34 | −1.924 | 22.069 | 3.592 | 1.00 | 119.16 | A | O |
| ATOM | 249 | N | PHE | A | 35 | −0.862 | 21.907 | 5.570 | 1.00 | 123.51 | A | N |
| ATOM | 250 | CA | PHE | A | 35 | −1.911 | 22.591 | 6.324 | 1.00 | 128.76 | A | C |
| ATOM | 251 | CB | PHE | A | 35 | −1.472 | 22.875 | 7.755 | 1.00 | 130.79 | A | C |
| ATOM | 252 | CG | PHE | A | 35 | −1.472 | 21.672 | 8.632 | 1.00 | 129.65 | A | C |
| ATOM | 253 | CD1 | PHE | A | 35 | −2.399 | 20.661 | 8.441 | 1.00 | 135.78 | A | C |
| ATOM | 254 | CE1 | PHE | A | 35 | −2.398 | 19.538 | 9.256 | 1.00 | 141.44 | A | C |
| ATOM | 255 | CZ | PHE | A | 35 | −1.476 | 19.425 | 10.286 | 1.00 | 135.01 | A | C |
| ATOM | 256 | CE2 | PHE | A | 35 | −0.556 | 20.437 | 10.492 | 1.00 | 158.44 | A | C |
| ATOM | 257 | CD2 | PHE | A | 35 | −0.559 | 21.552 | 9.667 | 1.00 | 148.38 | A | C |
| ATOM | 258 | C | PHE | A | 35 | −2.267 | 23.903 | 5.686 | 1.00 | 130.18 | A | C |
| ATOM | 259 | O | PHE | A | 35 | −3.435 | 24.201 | 5.469 | 1.00 | 138.97 | A | O |
| ATOM | 260 | N | VAL | A | 36 | −1.242 | 24.684 | 5.386 | 1.00 | 141.31 | A | N |
| ATOM | 261 | CA | VAL | A | 36 | −1.437 | 26.036 | 4.907 | 1.00 | 147.83 | A | C |
| ATOM | 262 | CB | VAL | A | 36 | −0.153 | 26.873 | 5.051 | 1.00 | 153.82 | A | C |
| ATOM | 263 | CG1 | VAL | A | 36 | −0.259 | 28.162 | 4.244 | 1.00 | 151.82 | A | C |
| ATOM | 264 | CG2 | VAL | A | 36 | 0.121 | 27.160 | 6.525 | 1.00 | 135.68 | A | C |
| ATOM | 265 | C | VAL | A | 36 | −1.950 | 26.027 | 3.475 | 1.00 | 143.48 | A | C |
| ATOM | 266 | O | VAL | A | 36 | −3.038 | 26.543 | 3.211 | 1.00 | 136.00 | A | O |
| ATOM | 267 | N | GLY | A | 37 | −1.172 | 25.432 | 2.567 | 1.00 | 137.90 | A | N |
| ATOM | 268 | CA | GLY | A | 37 | −1.607 | 25.225 | 1.186 | 1.00 | 142.69 | A | C |
| ATOM | 269 | C | GLY | A | 37 | −3.057 | 24.764 | 1.123 | 1.00 | 145.95 | A | C |
| ATOM | 270 | O | GLY | A | 37 | −3.867 | 25.324 | 0.373 | 1.00 | 128.51 | A | O |
| ATOM | 271 | N | GLY | A | 38 | −3.383 | 23.754 | 1.928 | 1.00 | 142.65 | A | N |
| ATOM | 272 | CA | GLY | A | 38 | −4.753 | 23.248 | 2.060 | 1.00 | 134.47 | A | C |
| ATOM | 273 | C | GLY | A | 38 | −5.703 | 24.355 | 2.460 | 1.00 | 120.90 | A | C |
| ATOM | 274 | O | GLY | A | 38 | −6.718 | 24.578 | 1.813 | 1.00 | 120.73 | A | O |
| ATOM | 275 | N | LEU | A | 39 | −5.359 | 25.061 | 3.525 | 1.00 | 116.89 | A | N |
| ATOM | 276 | CA | LEU | A | 39 | −6.154 | 26.182 | 3.978 | 1.00 | 126.65 | A | C |
| ATOM | 277 | CB | LEU | A | 39 | −5.469 | 26.859 | 5.151 | 1.00 | 124.69 | A | C |
| ATOM | 278 | CG | LEU | A | 39 | −6.272 | 27.960 | 5.821 | 1.00 | 150.74 | A | C |
| ATOM | 279 | CD1 | LEU | A | 39 | −7.663 | 27.482 | 6.237 | 1.00 | 172.35 | A | C |
| ATOM | 280 | CD2 | LEU | A | 39 | −5.505 | 28.513 | 7.014 | 1.00 | 144.52 | A | C |
| ATOM | 281 | C | LEU | A | 39 | −6.399 | 27.203 | 2.876 | 1.00 | 121.84 | A | C |
| ATOM | 282 | O | LEU | A | 39 | −7.538 | 27.530 | 2.589 | 1.00 | 121.18 | A | O |
| ATOM | 283 | N | SER | A | 40 | −5.335 | 27.704 | 2.257 | 1.00 | 115.93 | A | N |
| ATOM | 284 | CA | SER | A | 40 | −5.469 | 28.679 | 1.172 | 1.00 | 127.00 | A | C |
| ATOM | 285 | CB | SER | A | 40 | −4.174 | 28.820 | 0.384 | 1.00 | 133.03 | A | C |
| ATOM | 286 | OG | SER | A | 40 | −3.047 | 28.499 | 1.172 | 1.00 | 162.30 | A | O |
| ATOM | 287 | C | SER | A | 40 | −6.529 | 28.232 | 0.199 | 1.00 | 123.90 | A | C |
| ATOM | 288 | O | SER | A | 40 | −7.424 | 28.989 | −0.144 | 1.00 | 141.58 | A | O |
| ATOM | 289 | N | ILE | A | 41 | −6.421 | 26.986 | −0.236 | 1.00 | 116.87 | A | N |
| ATOM | 290 | CA | ILE | A | 41 | −7.170 | 26.499 | −1.376 | 1.00 | 112.73 | A | C |
| ATOM | 291 | CB | ILE | A | 41 | −6.552 | 25.196 | −1.897 | 1.00 | 111.17 | A | C |
| ATOM | 292 | CG1 | ILE | A | 41 | −5.208 | 25.517 | −2.536 | 1.00 | 114.24 | A | C |
| ATOM | 293 | CD1 | ILE | A | 41 | −4.349 | 24.293 | −2.697 | 1.00 | 130.59 | A | C |
| ATOM | 294 | CG2 | ILE | A | 41 | −7.462 | 24.470 | −2.881 | 1.00 | 119.28 | A | C |
| ATOM | 295 | C | ILE | A | 41 | −8.632 | 26.355 | −1.023 | 1.00 | 125.47 | A | C |
| ATOM | 296 | O | ILE | A | 41 | −9.501 | 26.701 | −1.821 | 1.00 | 127.73 | A | O |
| ATOM | 297 | N | VAL | A | 42 | −8.897 | 25.874 | 0.184 | 1.00 | 126.06 | A | N |
| ATOM | 298 | CA | VAL | A | 42 | −10.251 | 25.884 | 0.701 | 1.00 | 138.17 | A | C |
| ATOM | 299 | CB | VAL | A | 42 | −10.338 | 25.367 | 2.156 | 1.00 | 144.07 | A | C |
| ATOM | 300 | CG1 | VAL | A | 42 | −11.795 | 25.175 | 2.551 | 1.00 | 128.74 | A | C |
| ATOM | 301 | CG2 | VAL | A | 42 | −9.586 | 24.060 | 2.336 | 1.00 | 147.14 | A | C |
| ATOM | 302 | C | VAL | A | 42 | −10.788 | 27.310 | 0.657 | 1.00 | 123.89 | A | C |
| ATOM | 303 | O | VAL | A | 42 | −11.814 | 27.568 | 0.047 | 1.00 | 134.60 | A | O |
| ATOM | 304 | N | CYS | A | 43 | −10.080 | 28.236 | 1.293 | 1.00 | 122.79 | A | N |
| ATOM | 305 | CA | CYS | A | 43 | −10.535 | 29.620 | 1.388 | 1.00 | 128.14 | A | C |
| ATOM | 306 | CB | CYS | A | 43 | −9.455 | 30.485 | 2.017 | 1.00 | 125.68 | A | C |
| ATOM | 307 | SG | CYS | A | 43 | −9.109 | 29.981 | 3.703 | 1.00 | 157.34 | A | S |
| ATOM | 308 | C | CYS | A | 43 | −10.923 | 30.200 | 0.043 | 1.00 | 141.59 | A | C |
| ATOM | 309 | O | CYS | A | 43 | −12.072 | 30.597 | −0.171 | 1.00 | 139.08 | A | O |
| ATOM | 310 | N | ASN | A | 44 | −9.963 | 30.225 | −0.870 | 1.00 | 127.83 | A | N |
| ATOM | 311 | CA | ASN | A | 44 | −10.184 | 30.802 | −2.174 | 1.00 | 129.96 | A | C |
| ATOM | 312 | CB | ASN | A | 44 | −8.904 | 30.750 | −2.997 | 1.00 | 154.58 | A | C |
| ATOM | 313 | CG | ASN | A | 44 | −7.804 | 31.623 | −2.419 | 1.00 | 181.76 | A | C |
| ATOM | 314 | OD1 | ASN | A | 44 | −6.625 | 31.411 | −2.700 | 1.00 | 179.64 | A | O |
| ATOM | 315 | ND2 | ASN | A | 44 | −8.186 | 32.613 | −1.597 | 1.00 | 183.85 | A | N |
| ATOM | 316 | C | ASN | A | 44 | −11.333 | 30.138 | −2.909 | 1.00 | 137.62 | A | C |
| ATOM | 317 | O | ASN | A | 44 | −11.919 | 30.738 | −3.800 | 1.00 | 145.67 | A | O |
| ATOM | 318 | N | VAL | A | 45 | −11.675 | 28.918 | −2.508 | 1.00 | 129.90 | A | N |
| ATOM | 319 | CA | VAL | A | 45 | −12.779 | 28.195 | −3.128 | 1.00 | 132.34 | A | C |
| ATOM | 320 | CB | VAL | A | 45 | −12.645 | 26.686 | −2.902 | 1.00 | 124.68 | A | C |
| ATOM | 321 | CG1 | VAL | A | 45 | −14.009 | 26.016 | −2.782 | 1.00 | 124.51 | A | C |
| ATOM | 322 | CG2 | VAL | A | 45 | −11.809 | 26.081 | −4.021 | 1.00 | 134.72 | A | C |
| ATOM | 323 | C | VAL | A | 45 | −14.165 | 28.724 | −2.721 | 1.00 | 138.39 | A | C |
| ATOM | 324 | O | VAL | A | 45 | −15.037 | 28.928 | −3.578 | 1.00 | 133.47 | A | O |
| ATOM | 325 | N | VAL | A | 46 | −14.369 | 28.957 | −1.428 | 1.00 | 121.56 | A | N |
| ATOM | 326 | CA | VAL | A | 46 | −15.570 | 29.665 | −0.985 | 1.00 | 156.39 | A | C |
| ATOM | 327 | CB | VAL | A | 46 | −15.690 | 29.757 | 0.558 | 1.00 | 154.49 | A | C |
| ATOM | 328 | CG1 | VAL | A | 46 | −15.222 | 28.459 | 1.199 | 1.00 | 139.75 | A | C |
| ATOM | 329 | CG2 | VAL | A | 46 | −14.893 | 30.924 | 1.112 | 1.00 | 143.08 | A | C |
| ATOM | 330 | C | VAL | A | 46 | −15.556 | 31.047 | −1.639 | 1.00 | 150.90 | A | C |
| ATOM | 331 | O | VAL | A | 46 | −16.548 | 31.491 | −2.204 | 1.00 | 141.29 | A | O |
| ATOM | 332 | N | VAL | A | 47 | −14.396 | 31.691 | −1.606 | 1.00 | 145.57 | A | N |
| ATOM | 333 | CA | VAL | A | 47 | −14.212 | 33.008 | −2.185 | 1.00 | 141.47 | A | C |
| ATOM | 334 | CB | VAL | A | 47 | −12.793 | 33.508 | −1.909 | 1.00 | 137.07 | A | C |
| ATOM | 335 | CG1 | VAL | A | 47 | −12.486 | 34.739 | −2.730 | 1.00 | 135.06 | A | C |
| ATOM | 336 | CG2 | VAL | A | 47 | −12.612 | 33.786 | −0.428 | 1.00 | 153.44 | A | C |
| ATOM | 337 | C | VAL | A | 47 | −14.493 | 33.021 | −3.689 | 1.00 | 154.04 | A | C |
| ATOM | 338 | O | VAL | A | 47 | −15.112 | 33.946 | −4.195 | 1.00 | 156.29 | A | O |
| ATOM | 339 | N | ILE | A | 48 | −14.034 | 31.997 | −4.401 | 1.00 | 149.77 | A | N |
| ATOM | 340 | CA | ILE | A | 48 | −14.376 | 31.852 | −5.812 | 1.00 | 150.12 | A | C |
| ATOM | 341 | CB | ILE | A | 48 | −13.701 | 30.643 | −6.468 | 1.00 | 155.05 | A | C |
| ATOM | 342 | CG1 | ILE | A | 48 | −12.223 | 30.928 | −6.707 | 1.00 | 168.50 | A | C |
| ATOM | 343 | CD1 | ILE | A | 48 | −11.395 | 29.671 | −6.805 | 1.00 | 159.99 | A | C |
| ATOM | 344 | CG2 | ILE | A | 48 | −14.384 | 30.308 | −7.792 | 1.00 | 162.97 | A | C |
| ATOM | 345 | C | ILE | A | 48 | −15.871 | 31.679 | −5.967 | 1.00 | 159.98 | A | C |
| ATOM | 346 | O | ILE | A | 48 | −16.475 | 32.301 | −6.838 | 1.00 | 158.52 | A | O |
| ATOM | 347 | N | THR | A | 49 | −16.460 | 30.828 | −5.127 | 1.00 | 158.84 | A | N |
| ATOM | 348 | CA | THR | A | 49 | −17.890 | 30.560 | −5.223 | 1.00 | 142.96 | A | C |
| ATOM | 349 | CB | THR | A | 49 | −18.328 | 29.350 | −4.396 | 1.00 | 132.73 | A | C |
| ATOM | 350 | OG1 | THR | A | 49 | −17.938 | 29.545 | −3.032 | 1.00 | 171.55 | A | O |
| ATOM | 351 | CG2 | THR | A | 49 | −17.692 | 28.094 | −4.938 | 1.00 | 102.52 | A | C |
| ATOM | 352 | C | THR | A | 49 | −18.705 | 31.783 | −4.824 | 1.00 | 153.97 | A | C |
| ATOM | 353 | O | THR | A | 49 | −19.770 | 32.003 | −5.363 | 1.00 | 173.10 | A | O |
| ATOM | 354 | N | TYR | A | 50 | −18.186 | 32.589 | −3.904 | 1.00 | 165.86 | A | N |
| ATOM | 355 | CA | TYR | A | 50 | −18.905 | 33.761 | −3.429 | 1.00 | 165.43 | A | C |
| ATOM | 356 | CB | TYR | A | 50 | −18.270 | 34.351 | −2.182 | 1.00 | 175.74 | A | C |
| ATOM | 357 | CG | TYR | A | 50 | −18.829 | 33.753 | −0.935 | 1.00 | 230.91 | A | C |
| ATOM | 358 | CD1 | TYR | A | 50 | −18.243 | 32.615 | −0.376 | 1.00 | 236.49 | A | C |
| ATOM | 359 | CE1 | TYR | A | 50 | −18.754 | 32.043 | 0.779 | 1.00 | 280.12 | A | C |
| ATOM | 360 | CZ | TYR | A | 50 | −19.886 | 32.611 | 1.381 | 1.00 | 324.66 | A | C |
| ATOM | 361 | OH | TYR | A | 50 | −20.418 | 32.063 | 2.532 | 1.00 | 310.38 | A | O |
| ATOM | 362 | CE2 | TYR | A | 50 | −20.498 | 33.729 | 0.825 | 1.00 | 251.84 | A | C |
| ATOM | 363 | CD2 | TYR | A | 50 | −19.966 | 34.298 | −0.323 | 1.00 | 234.71 | A | C |
| ATOM | 364 | C | TYR | A | 50 | −19.034 | 34.850 | −4.448 | 1.00 | 163.29 | A | C |
| ATOM | 365 | O | TYR | A | 50 | −19.984 | 35.625 | −4.388 | 1.00 | 185.24 | A | O |
| ATOM | 366 | N | SER | A | 51 | −18.071 | 34.947 | −5.353 | 1.00 | 156.23 | A | N |
| ATOM | 367 | CA | SER | A | 51 | −18.183 | 35.867 | −6.463 | 1.00 | 183.70 | A | C |
| ATOM | 368 | CB | SER | A | 51 | −16.813 | 36.409 | −6.872 | 1.00 | 195.60 | A | C |
| ATOM | 369 | OG | SER | A | 51 | −15.883 | 35.351 | −7.000 | 1.00 | 207.20 | A | O |
| ATOM | 370 | C | SER | A | 51 | −18.904 | 35.184 | −7.619 | 1.00 | 182.19 | A | C |
| ATOM | 371 | O | SER | A | 51 | −19.679 | 35.812 | −8.337 | 1.00 | 216.12 | A | O |
| ATOM | 372 | N | ALA | A | 52 | −18.672 | 33.888 | −7.780 | 1.00 | 163.06 | A | N |
| ATOM | 373 | CA | ALA | A | 52 | −19.520 | 33.091 | −8.655 | 1.00 | 178.32 | A | C |
| ATOM | 374 | CB | ALA | A | 52 | −19.136 | 31.626 | −8.578 | 1.00 | 162.25 | A | C |
| ATOM | 375 | C | ALA | A | 52 | −20.998 | 33.290 | −8.283 | 1.00 | 192.45 | A | C |
| ATOM | 376 | O | ALA | A | 52 | −21.873 | 33.203 | −9.145 | 1.00 | 208.33 | A | O |
| ATOM | 377 | N | LEU | A | 53 | −21.251 | 33.559 | −6.997 | 1.00 | 191.87 | A | N |
| ATOM | 378 | CA | LEU | A | 53 | −22.580 | 33.932 | −6.494 | 1.00 | 208.85 | A | C |
| ATOM | 379 | CB | LEU | A | 53 | −22.583 | 34.043 | −4.960 | 1.00 | 248.92 | A | C |
| ATOM | 380 | CG | LEU | A | 53 | −23.098 | 32.911 | −4.069 | 1.00 | 242.85 | A | C |
| ATOM | 381 | CD1 | LEU | A | 53 | −22.271 | 32.841 | −2.789 | 1.00 | 190.70 | A | C |
| ATOM | 382 | CD2 | LEU | A | 53 | −24.591 | 33.072 | −3.777 | 1.00 | 315.38 | A | C |
| ATOM | 383 | C | LEU | A | 53 | −23.017 | 35.263 | −7.048 | 1.00 | 207.80 | A | C |
| ATOM | 384 | O | LEU | A | 53 | −24.100 | 35.381 | −7.621 | 1.00 | 209.79 | A | O |
| ATOM | 385 | N | HIS | A | 54 | −22.161 | 36.262 | −6.862 | 1.00 | 211.10 | A | N |
| ATOM | 386 | CA | HIS | A | 54 | −22.528 | 37.640 | −7.117 | 1.00 | 237.71 | A | C |
| ATOM | 387 | CB | HIS | A | 54 | −22.356 | 38.489 | −5.855 | 1.00 | 260.20 | A | C |
| ATOM | 388 | CG | HIS | A | 54 | −23.007 | 37.892 | −4.621 | 1.00 | 268.59 | A | C |
| ATOM | 389 | ND1 | HIS | A | 54 | −22.301 | 37.273 | −3.649 | 1.00 | 232.20 | A | N |
| ATOM | 390 | CE1 | HIS | A | 54 | −23.142 | 36.845 | −2.689 | 1.00 | 248.23 | A | C |
| ATOM | 391 | NE2 | HIS | A | 54 | −24.393 | 37.185 | −3.047 | 1.00 | 280.87 | A | N |
| ATOM | 392 | CD2 | HIS | A | 54 | −24.345 | 37.829 | −4.234 | 1.00 | 293.38 | A | C |
| ATOM | 393 | C | HIS | A | 54 | −21.665 | 38.144 | −8.219 | 1.00 | 254.00 | A | C |
| ATOM | 394 | O | HIS | A | 54 | −20.569 | 38.651 | −7.956 | 1.00 | 237.38 | A | O |
| ATOM | 395 | N | PRO | A | 55 | −22.127 | 37.980 | −9.479 | 1.00 | 280.33 | A | N |
| ATOM | 396 | CA | PRO | A | 55 | −21.378 | 38.435 | −10.656 | 1.00 | 289.10 | A | C |
| ATOM | 397 | CB | PRO | A | 55 | −22.309 | 38.080 | −11.827 | 1.00 | 303.21 | A | C |
| ATOM | 398 | CG | PRO | A | 55 | −23.199 | 36.994 | −11.318 | 1.00 | 230.23 | A | C |
| ATOM | 399 | CD | PRO | A | 55 | −23.372 | 37.277 | −9.853 | 1.00 | 267.16 | A | C |
| ATOM | 400 | C | PRO | A | 55 | −21.096 | 39.944 | −10.618 | 1.00 | 304.75 | A | C |
| ATOM | 401 | O | PRO | A | 55 | −20.850 | 40.548 | −11.658 | 1.00 | 315.84 | A | O |
| ATOM | 402 | N | THR | A | 56 | −21.106 | 40.523 | −9.415 | 1.00 | 309.22 | A | N |
| ATOM | 403 | CA | THR | A | 56 | −21.147 | 41.970 | −9.212 | 1.00 | 302.16 | A | C |
| ATOM | 404 | CB | THR | A | 56 | −19.761 | 42.637 | −9.396 | 1.00 | 237.96 | A | C |
| ATOM | 405 | OG1 | THR | A | 56 | −19.359 | 42.530 | −10.765 | 1.00 | 345.81 | A | O |
| ATOM | 406 | CG2 | THR | A | 56 | −18.700 | 41.990 | −8.506 | 1.00 | 196.02 | A | C |
| ATOM | 407 | C | THR | A | 56 | −22.211 | 42.607 | −10.129 | 1.00 | 306.97 | A | C |
| ATOM | 408 | O | THR | A | 56 | −22.145 | 43.792 | −10.453 | 1.00 | 346.45 | A | O |
| ATOM | 409 | N | ALA | A | 57 | −23.181 | 41.795 | −10.554 | 1.00 | 338.20 | A | N |
| ATOM | 410 | CA | ALA | A | 57 | −24.355 | 42.267 | −11.292 | 1.00 | 335.27 | A | C |
| ATOM | 411 | CB | ALA | A | 57 | −24.893 | 41.182 | −12.218 | 1.00 | 304.22 | A | C |
| ATOM | 412 | C | ALA | A | 57 | −25.448 | 42.744 | −10.329 | 1.00 | 365.36 | A | C |
| ATOM | 413 | O | ALA | A | 57 | −26.266 | 43.604 | −10.712 | 1.00 | 320.43 | A | O |
| ATOM | 414 | N | PRO | A | 58 | −25.478 | 42.176 | −9.089 | 1.00 | 387.74 | A | N |
| ATOM | 415 | CA | PRO | A | 58 | −26.370 | 42.656 | −8.030 | 1.00 | 372.96 | A | C |
| ATOM | 416 | CB | PRO | A | 58 | −26.104 | 41.679 | −6.868 | 1.00 | 265.06 | A | C |
| ATOM | 417 | CG | PRO | A | 58 | −24.767 | 41.070 | −7.153 | 1.00 | 262.65 | A | C |
| ATOM | 418 | CD | PRO | A | 58 | −24.757 | 40.961 | −8.651 | 1.00 | 319.73 | A | C |
| ATOM | 419 | C | PRO | A | 58 | −26.038 | 44.083 | −7.600 | 1.00 | 328.83 | A | C |
| ATOM | 420 | O | PRO | A | 58 | −26.714 | 44.639 | −6.733 | 1.00 | 321.02 | A | O |
| ATOM | 421 | N | SER | A | 74 | −12.165 | 42.455 | −9.780 | 1.00 | 197.82 | A | N |
| ATOM | 422 | CA | SER | A | 74 | −12.824 | 41.154 | −9.981 | 1.00 | 202.13 | A | C |
| ATOM | 423 | CB | SER | A | 74 | −12.453 | 40.529 | −11.344 | 1.00 | 173.61 | A | C |
| ATOM | 424 | OG | SER | A | 74 | −12.125 | 41.490 | −12.323 | 1.00 | 135.39 | A | O |
| ATOM | 425 | C | SER | A | 74 | −12.465 | 40.167 | −8.872 | 1.00 | 171.00 | A | C |
| ATOM | 426 | O | SER | A | 74 | −12.170 | 40.562 | −7.738 | 1.00 | 176.52 | A | O |
| ATOM | 427 | N | PHE | A | 75 | −12.509 | 38.879 | −9.207 | 1.00 | 167.19 | A | N |
| ATOM | 428 | CA | PHE | A | 75 | −11.930 | 37.831 | −8.359 | 1.00 | 166.66 | A | C |
| ATOM | 429 | CB | PHE | A | 75 | −12.632 | 36.488 | −8.575 | 1.00 | 162.43 | A | C |
| ATOM | 430 | CG | PHE | A | 75 | −12.821 | 36.090 | −10.021 | 1.00 | 185.68 | A | C |
| ATOM | 431 | CD1 | PHE | A | 75 | −11.743 | 35.669 | −10.818 | 1.00 | 204.66 | A | C |
| ATOM | 432 | CE1 | PHE | A | 75 | −11.939 | 35.297 | −12.142 | 1.00 | 192.89 | A | C |
| ATOM | 433 | CZ | PHE | A | 75 | −13.220 | 35.307 | −12.672 | 1.00 | 168.34 | A | C |
| ATOM | 434 | CE2 | PHE | A | 75 | −14.300 | 35.686 | −11.885 | 1.00 | 182.85 | A | C |
| ATOM | 435 | CD2 | PHE | A | 75 | −14.100 | 36.068 | −10.569 | 1.00 | 178.46 | A | C |
| ATOM | 436 | C | PHE | A | 75 | −10.409 | 37.685 | −8.547 | 1.00 | 162.88 | A | C |
| ATOM | 437 | O | PHE | A | 75 | −9.793 | 36.665 | −8.196 | 1.00 | 136.58 | A | O |
| ATOM | 438 | N | TYR | A | 76 | −9.811 | 38.723 | −9.117 | 1.00 | 165.03 | A | N |
| ATOM | 439 | CA | TYR | A | 76 | −8.363 | 38.877 | −9.166 | 1.00 | 174.01 | A | C |
| ATOM | 440 | CB | TYR | A | 76 | −8.040 | 40.343 | −9.479 | 1.00 | 198.26 | A | C |
| ATOM | 441 | CG | TYR | A | 76 | −6.620 | 40.791 | −9.201 | 1.00 | 209.25 | A | C |
| ATOM | 442 | CD1 | TYR | A | 76 | −5.603 | 40.594 | −10.144 | 1.00 | 229.03 | A | C |
| ATOM | 443 | CE1 | TYR | A | 76 | −4.307 | 41.025 | −9.896 | 1.00 | 247.22 | A | C |
| ATOM | 444 | CZ | TYR | A | 76 | −4.019 | 41.680 | −8.697 | 1.00 | 240.12 | A | C |
| ATOM | 445 | OH | TYR | A | 76 | −2.741 | 42.120 | −8.435 | 1.00 | 260.02 | A | O |
| ATOM | 446 | CE2 | TYR | A | 76 | −5.014 | 41.890 | −7.754 | 1.00 | 221.13 | A | C |
| ATOM | 447 | CD2 | TYR | A | 76 | −6.305 | 41.454 | −8.014 | 1.00 | 202.82 | A | C |
| ATOM | 448 | C | TYR | A | 76 | −7.704 | 38.446 | −7.853 | 1.00 | 186.44 | A | C |
| ATOM | 449 | O | TYR | A | 76 | −6.566 | 37.972 | −7.854 | 1.00 | 189.07 | A | O |
| ATOM | 450 | N | GLY | A | 77 | −8.427 | 38.629 | −6.745 | 1.00 | 194.24 | A | N |
| ATOM | 451 | CA | GLY | A | 77 | −7.927 | 38.329 | −5.397 | 1.00 | 171.94 | A | C |
| ATOM | 452 | C | GLY | A | 77 | −7.600 | 36.863 | −5.187 | 1.00 | 164.22 | A | C |
| ATOM | 453 | O | GLY | A | 77 | −6.493 | 36.529 | −4.770 | 1.00 | 161.49 | A | O |
| ATOM | 454 | N | PRO | A | 78 | −8.569 | 35.980 | −5.461 | 1.00 | 167.57 | A | N |
| ATOM | 455 | CA | PRO | A | 78 | −8.330 | 34.537 | −5.479 | 1.00 | 167.78 | A | C |
| ATOM | 456 | CB | PRO | A | 78 | −9.623 | 33.977 | −6.063 | 1.00 | 151.63 | A | C |
| ATOM | 457 | CG | PRO | A | 78 | −10.661 | 34.943 | −5.628 | 1.00 | 160.26 | A | C |
| ATOM | 458 | CD | PRO | A | 78 | −10.004 | 36.291 | −5.439 | 1.00 | 169.11 | A | C |
| ATOM | 459 | C | PRO | A | 78 | −7.175 | 34.154 | −6.377 | 1.00 | 152.95 | A | C |
| ATOM | 460 | O | PRO | A | 78 | −6.381 | 33.288 | −6.001 | 1.00 | 157.92 | A | O |
| ATOM | 461 | N | ALA | A | 79 | −7.093 | 34.775 | −7.555 | 1.00 | 153.02 | A | N |
| ATOM | 462 | CA | ALA | A | 79 | −5.984 | 34.537 | −8.476 | 1.00 | 149.96 | A | C |
| ATOM | 463 | CB | ALA | A | 79 | −6.078 | 35.428 | −9.702 | 1.00 | 152.59 | A | C |
| ATOM | 464 | C | ALA | A | 79 | −4.677 | 34.758 | −7.742 | 1.00 | 162.36 | A | C |
| ATOM | 465 | O | ALA | A | 79 | −3.834 | 33.870 | −7.676 | 1.00 | 147.12 | A | O |
| ATOM | 466 | N | THR | A | 80 | −4.532 | 35.939 | −7.157 | 1.00 | 175.53 | A | N |
| ATOM | 467 | CA | THR | A | 80 | −3.372 | 36.253 | −6.333 | 1.00 | 161.34 | A | C |
| ATOM | 468 | CB | THR | A | 80 | −3.388 | 37.720 | −5.869 | 1.00 | 173.95 | A | C |
| ATOM | 469 | OG1 | THR | A | 80 | −4.528 | 37.949 | −5.029 | 1.00 | 194.62 | A | O |
| ATOM | 470 | CG2 | THR | A | 80 | −3.443 | 38.650 | −7.056 | 1.00 | 179.32 | A | C |
| ATOM | 471 | C | THR | A | 80 | −3.300 | 35.338 | −5.106 | 1.00 | 175.40 | A | C |
| ATOM | 472 | O | THR | A | 80 | −2.221 | 34.871 | −4.736 | 1.00 | 167.58 | A | O |
| ATOM | 473 | N | GLY | A | 81 | −4.452 | 35.088 | −4.490 | 1.00 | 166.46 | A | N |
| ATOM | 474 | CA | GLY | A | 81 | −4.509 | 34.276 | −3.284 | 1.00 | 155.01 | A | C |
| ATOM | 475 | C | GLY | A | 81 | −3.863 | 32.919 | −3.451 | 1.00 | 161.20 | A | C |
| ATOM | 476 | O | GLY | A | 81 | −2.799 | 32.645 | −2.873 | 1.00 | 164.44 | A | O |
| ATOM | 477 | N | LEU | A | 82 | −4.500 | 32.078 | −4.268 | 1.00 | 168.41 | A | N |
| ATOM | 478 | CA | LEU | A | 82 | −4.082 | 30.687 | −4.424 | 1.00 | 159.57 | A | C |
| ATOM | 479 | CB | LEU | A | 82 | −5.247 | 29.817 | −4.903 | 1.00 | 133.78 | A | C |
| ATOM | 480 | CG | LEU | A | 82 | −5.843 | 30.025 | −6.288 | 1.00 | 144.40 | A | C |
| ATOM | 481 | CD1 | LEU | A | 82 | −5.211 | 29.029 | −7.238 | 1.00 | 109.34 | A | C |
| ATOM | 482 | CD2 | LEU | A | 82 | −7.329 | 29.752 | −6.225 | 1.00 | 141.80 | A | C |
| ATOM | 483 | C | LEU | A | 82 | −2.851 | 30.572 | −5.325 | 1.00 | 143.61 | A | C |
| ATOM | 484 | O | LEU | A | 82 | −2.345 | 29.475 | −5.608 | 1.00 | 137.05 | A | O |
| ATOM | 485 | N | LEU | A | 83 | −2.364 | 31.719 | −5.762 | 1.00 | 135.19 | A | N |
| ATOM | 486 | CA | LEU | A | 83 | −1.064 | 31.774 | −6.387 | 1.00 | 145.26 | A | C |
| ATOM | 487 | CB | LEU | A | 83 | −0.692 | 33.211 | −6.711 | 1.00 | 134.37 | A | C |
| ATOM | 488 | CG | LEU | A | 83 | 0.513 | 33.288 | −7.622 | 1.00 | 175.50 | A | C |
| ATOM | 489 | CD1 | LEU | A | 83 | 0.193 | 32.653 | −8.973 | 1.00 | 181.99 | A | C |
| ATOM | 490 | CD2 | LEU | A | 83 | 0.925 | 34.745 | −7.757 | 1.00 | 206.17 | A | C |
| ATOM | 491 | C | LEU | A | 83 | −0.000 | 31.131 | −5.500 | 1.00 | 146.57 | A | C |
| ATOM | 492 | O | LEU | A | 83 | 0.621 | 30.141 | −5.888 | 1.00 | 149.56 | A | O |
| ATOM | 493 | N | PHE | A | 84 | 0.203 | 31.686 | −4.310 | 1.00 | 125.67 | A | N |
| ATOM | 494 | CA | PHE | A | 84 | 1.125 | 31.064 | −3.375 | 1.00 | 157.55 | A | C |
| ATOM | 495 | CB | PHE | A | 84 | 1.989 | 32.087 | −2.626 | 1.00 | 161.15 | A | C |
| ATOM | 496 | CG | PHE | A | 84 | 1.245 | 33.271 | −2.113 | 1.00 | 168.59 | A | C |
| ATOM | 497 | CD1 | PHE | A | 84 | 0.690 | 33.253 | −0.839 | 1.00 | 175.91 | A | C |
| ATOM | 498 | CE1 | PHE | A | 84 | 0.009 | 34.356 | −0.348 | 1.00 | 217.88 | A | C |
| ATOM | 499 | CZ | PHE | A | 84 | −0.109 | 35.496 | −1.129 | 1.00 | 232.82 | A | C |
| ATOM | 500 | CE2 | PHE | A | 84 | 0.457 | 35.527 | −2.398 | 1.00 | 204.69 | A | C |
| ATOM | 501 | CD2 | PHE | A | 84 | 1.138 | 34.423 | −2.884 | 1.00 | 158.13 | A | C |
| ATOM | 502 | C | PHE | A | 84 | 0.460 | 30.062 | −2.434 | 1.00 | 162.61 | A | C |
| ATOM | 503 | O | PHE | A | 84 | 1.131 | 29.395 | −1.641 | 1.00 | 166.86 | A | O |
| ATOM | 504 | N | GLY | A | 85 | −0.857 | 29.941 | −2.543 | 1.00 | 156.57 | A | N |
| ATOM | 505 | CA | GLY | A | 85 | −1.551 | 28.774 | −2.009 | 1.00 | 158.97 | A | C |
| ATOM | 506 | C | GLY | A | 85 | −0.936 | 27.490 | −2.549 | 1.00 | 153.04 | A | C |
| ATOM | 507 | O | GLY | A | 85 | −0.547 | 26.610 | −1.775 | 1.00 | 135.79 | A | O |
| ATOM | 508 | N | PHE | A | 86 | −0.832 | 27.401 | −3.877 | 1.00 | 132.83 | A | N |
| ATOM | 509 | CA | PHE | A | 86 | −0.226 | 26.258 | −4.540 | 1.00 | 123.33 | A | C |
| ATOM | 510 | CB | PHE | A | 86 | −0.314 | 26.401 | −6.052 | 1.00 | 120.12 | A | C |
| ATOM | 511 | CG | PHE | A | 86 | −1.661 | 26.096 | −6.598 | 1.00 | 113.20 | A | C |
| ATOM | 512 | CD1 | PHE | A | 86 | −2.567 | 25.342 | −5.857 | 1.00 | 119.95 | A | C |
| ATOM | 513 | CE1 | PHE | A | 86 | −3.822 | 25.043 | −6.364 | 1.00 | 118.00 | A | C |
| ATOM | 514 | CZ | PHE | A | 86 | −4.185 | 25.490 | −7.629 | 1.00 | 107.65 | A | C |
| ATOM | 515 | CE2 | PHE | A | 86 | −3.282 | 26.235 | −8.379 | 1.00 | 126.15 | A | C |
| ATOM | 516 | CD2 | PHE | A | 86 | −2.027 | 26.530 | −7.863 | 1.00 | 124.54 | A | C |
| ATOM | 517 | C | PHE | A | 86 | 1.220 | 26.107 | −4.158 | 1.00 | 116.96 | A | C |
| ATOM | 518 | O | PHE | A | 86 | 1.680 | 25.008 | −3.912 | 1.00 | 115.25 | A | O |
| ATOM | 519 | N | THR | A | 87 | 1.932 | 27.223 | −4.104 | 1.00 | 122.59 | A | N |
| ATOM | 520 | CA | THR | A | 87 | 3.354 | 27.197 | −3.828 | 1.00 | 126.92 | A | C |
| ATOM | 521 | CB | THR | A | 87 | 3.908 | 28.605 | −3.590 | 1.00 | 141.51 | A | C |
| ATOM | 522 | OG1 | THR | A | 87 | 3.773 | 29.369 | −4.798 | 1.00 | 160.76 | A | O |
| ATOM | 523 | CG2 | THR | A | 87 | 5.391 | 28.531 | −3.159 | 1.00 | 131.88 | A | C |
| ATOM | 524 | C | THR | A | 87 | 3.667 | 26.341 | −2.624 | 1.00 | 122.46 | A | C |
| ATOM | 525 | O | THR | A | 87 | 4.657 | 25.616 | −2.615 | 1.00 | 130.38 | A | O |
| ATOM | 526 | N | TYR | A | 88 | 2.816 | 26.418 | −1.611 | 1.00 | 119.53 | A | N |
| ATOM | 527 | CA | TYR | A | 88 | 3.037 | 25.660 | −0.397 | 1.00 | 111.61 | A | C |
| ATOM | 528 | CB | TYR | A | 88 | 2.210 | 26.241 | 0.730 | 1.00 | 137.05 | A | C |
| ATOM | 529 | CG | TYR | A | 88 | 2.634 | 27.642 | 1.089 | 1.00 | 149.13 | A | C |
| ATOM | 530 | CD1 | TYR | A | 88 | 3.951 | 27.915 | 1.476 | 1.00 | 133.00 | A | C |
| ATOM | 531 | CE1 | TYR | A | 88 | 4.352 | 29.200 | 1.809 | 1.00 | 168.96 | A | C |
| ATOM | 532 | CZ | TYR | A | 88 | 3.439 | 30.232 | 1.755 | 1.00 | 194.66 | A | C |
| ATOM | 533 | OH | TYR | A | 88 | 3.835 | 31.520 | 2.091 | 1.00 | 207.47 | A | O |
| ATOM | 534 | CE2 | TYR | A | 88 | 2.122 | 29.985 | 1.369 | 1.00 | 182.23 | A | C |
| ATOM | 535 | CD2 | TYR | A | 88 | 1.729 | 28.694 | 1.042 | 1.00 | 159.01 | A | C |
| ATOM | 536 | C | TYR | A | 88 | 2.709 | 24.218 | −0.615 | 1.00 | 107.58 | A | C |
| ATOM | 537 | O | TYR | A | 88 | 3.504 | 23.343 | −0.298 | 1.00 | 116.97 | A | O |
| ATOM | 538 | N | LEU | A | 89 | 1.533 | 23.981 | −1.181 | 1.00 | 119.21 | A | N |
| ATOM | 539 | CA | LEU | A | 89 | 1.113 | 22.649 | −1.570 | 1.00 | 109.42 | A | C |
| ATOM | 540 | CB | LEU | A | 89 | −0.302 | 22.694 | −2.148 | 1.00 | 105.36 | A | C |
| ATOM | 541 | CG | LEU | A | 89 | −0.950 | 21.365 | −2.496 | 1.00 | 98.54 | A | C |
| ATOM | 542 | CD1 | LEU | A | 89 | −0.771 | 20.392 | −1.365 | 1.00 | 112.06 | A | C |
| ATOM | 543 | CD2 | LEU | A | 89 | −2.423 | 21.535 | −2.735 | 1.00 | 99.15 | A | C |
| ATOM | 544 | C | LEU | A | 89 | 2.106 | 22.006 | −2.547 | 1.00 | 112.56 | A | C |
| ATOM | 545 | O | LEU | A | 89 | 2.401 | 20.821 | −2.440 | 1.00 | 107.24 | A | O |
| ATOM | 546 | N | TYR | A | 90 | 2.624 | 22.792 | −3.484 | 1.00 | 106.81 | A | N |
| ATOM | 547 | CA | TYR | A | 90 | 3.692 | 22.352 | −4.369 | 1.00 | 115.56 | A | C |
| ATOM | 548 | CB | TYR | A | 90 | 4.160 | 23.483 | −5.301 | 1.00 | 115.05 | A | C |
| ATOM | 549 | CG | TYR | A | 90 | 4.529 | 23.005 | −6.692 | 1.00 | 128.38 | A | C |
| ATOM | 550 | CD1 | TYR | A | 90 | 5.526 | 22.048 | −6.895 | 1.00 | 128.66 | A | C |
| ATOM | 551 | CE1 | TYR | A | 90 | 5.851 | 21.601 | −8.175 | 1.00 | 151.57 | A | C |
| ATOM | 552 | CZ | TYR | A | 90 | 5.174 | 22.103 | −9.267 | 1.00 | 152.79 | A | C |
| ATOM | 553 | OH | TYR | A | 90 | 5.491 | 21.660 | −10.535 | 1.00 | 184.57 | A | O |
| ATOM | 554 | CE2 | TYR | A | 90 | 4.186 | 23.051 | −9.089 | 1.00 | 156.69 | A | C |
| ATOM | 555 | CD2 | TYR | A | 90 | 3.867 | 23.497 | −7.812 | 1.00 | 150.44 | A | C |
| ATOM | 556 | C | TYR | A | 90 | 4.875 | 21.870 | −3.545 | 1.00 | 121.14 | A | C |
| ATOM | 557 | O | TYR | A | 90 | 5.435 | 20.811 | −3.822 | 1.00 | 123.89 | A | O |
| ATOM | 558 | N | ALA | A | 91 | 5.256 | 22.653 | −2.540 | 1.00 | 108.83 | A | N |
| ATOM | 559 | CA | ALA | A | 91 | 6.351 | 22.277 | −1.674 | 1.00 | 112.70 | A | C |
| ATOM | 560 | CB | ALA | A | 91 | 6.589 | 23.346 | −0.651 | 1.00 | 105.51 | A | C |
| ATOM | 561 | C | ALA | A | 91 | 6.024 | 20.983 | −0.970 | 1.00 | 109.62 | A | C |
| ATOM | 562 | O | ALA | A | 91 | 6.789 | 20.021 | −1.028 | 1.00 | 123.54 | A | O |
| ATOM | 563 | N | ALA | A | 92 | 4.877 | 20.962 | −0.307 | 1.00 | 112.24 | A | N |
| ATOM | 564 | CA | ALA | A | 92 | 4.500 | 19.837 | 0.519 | 1.00 | 111.05 | A | C |
| ATOM | 565 | CB | ALA | A | 92 | 3.078 | 20.010 | 1.022 | 1.00 | 104.90 | A | C |
| ATOM | 566 | C | ALA | A | 92 | 4.648 | 18.565 | −0.286 | 1.00 | 112.99 | A | C |
| ATOM | 567 | O | ALA | A | 92 | 5.297 | 17.601 | 0.153 | 1.00 | 109.87 | A | O |
| ATOM | 568 | N | ILE | A | 93 | 4.078 | 18.588 | −1.483 | 1.00 | 99.98 | A | N |
| ATOM | 569 | CA | ILE | A | 93 | 4.048 | 17.402 | −2.310 | 1.00 | 107.21 | A | C |
| ATOM | 570 | CB | ILE | A | 93 | 3.036 | 17.551 | −3.442 | 1.00 | 96.25 | A | C |
| ATOM | 571 | CG1 | ILE | A | 93 | 1.633 | 17.398 | −2.853 | 1.00 | 99.91 | A | C |
| ATOM | 572 | CD1 | ILE | A | 93 | 0.515 | 17.581 | −3.856 | 1.00 | 130.95 | A | C |
| ATOM | 573 | CG2 | ILE | A | 93 | 3.295 | 16.530 | −4.541 | 1.00 | 109.19 | A | C |
| ATOM | 574 | C | ILE | A | 93 | 5.435 | 17.022 | −2.816 | 1.00 | 116.92 | A | C |
| ATOM | 575 | O | ILE | A | 93 | 5.850 | 15.874 | −2.686 | 1.00 | 115.47 | A | O |
| ATOM | 576 | N | ASN | A | 94 | 6.161 | 17.990 | −3.364 | 1.00 | 124.67 | A | N |
| ATOM | 577 | CA | ASN | A | 94 | 7.536 | 17.745 | −3.827 | 1.00 | 134.75 | A | C |
| ATOM | 578 | CB | ASN | A | 94 | 8.156 | 19.011 | −4.425 | 1.00 | 138.54 | A | C |
| ATOM | 579 | CG | ASN | A | 94 | 8.028 | 19.084 | −5.944 | 1.00 | 140.95 | A | C |
| ATOM | 580 | OD1 | ASN | A | 94 | 7.451 | 18.205 | −6.585 | 1.00 | 141.32 | A | O |
| ATOM | 581 | ND2 | ASN | A | 94 | 8.572 | 20.140 | −6.530 | 1.00 | 145.16 | A | N |
| ATOM | 582 | C | ASN | A | 94 | 8.445 | 17.144 | −2.742 | 1.00 | 125.82 | A | C |
| ATOM | 583 | O | ASN | A | 94 | 9.349 | 16.358 | −3.048 | 1.00 | 123.46 | A | O |
| ATOM | 584 | N | HIS | A | 95 | 8.185 | 17.507 | −1.483 | 1.00 | 125.90 | A | N |
| ATOM | 585 | CA | HIS | A | 95 | 8.955 | 17.000 | −0.342 | 1.00 | 106.62 | A | C |
| ATOM | 586 | CB | HIS | A | 95 | 8.764 | 17.872 | 0.873 | 1.00 | 97.70 | A | C |
| ATOM | 587 | CG | HIS | A | 95 | 9.828 | 18.895 | 1.033 | 1.00 | 124.24 | A | C |
| ATOM | 588 | ND1 | HIS | A | 95 | 9.563 | 20.147 | 1.435 | 1.00 | 146.85 | A | N |
| ATOM | 589 | CE1 | HIS | A | 95 | 10.708 | 20.849 | 1.480 | 1.00 | 155.98 | A | C |
| ATOM | 590 | NE2 | HIS | A | 95 | 11.716 | 20.046 | 1.086 | 1.00 | 217.03 | A | N |
| ATOM | 591 | CD2 | HIS | A | 95 | 11.209 | 18.831 | 0.802 | 1.00 | 137.13 | A | C |
| ATOM | 592 | C | HIS | A | 95 | 8.591 | 15.618 | 0.054 | 1.00 | 117.88 | A | C |
| ATOM | 593 | O | HIS | A | 95 | 9.460 | 14.757 | 0.196 | 1.00 | 126.78 | A | O |
| ATOM | 594 | N | THR | A | 96 | 7.302 | 15.405 | 0.285 | 1.00 | 116.99 | A | N |
| ATOM | 595 | CA | THR | A | 96 | 6.848 | 14.111 | 0.753 | 1.00 | 109.61 | A | C |
| ATOM | 596 | CB | THR | A | 96 | 5.345 | 14.095 | 1.070 | 1.00 | 109.01 | A | C |
| ATOM | 597 | OG1 | THR | A | 96 | 4.656 | 14.835 | 0.065 | 1.00 | 117.28 | A | O |
| ATOM | 598 | CG2 | THR | A | 96 | 5.091 | 14.764 | 2.369 | 1.00 | 100.56 | A | C |
| ATOM | 599 | C | THR | A | 96 | 7.175 | 13.053 | −0.279 | 1.00 | 121.34 | A | C |
| ATOM | 600 | O | THR | A | 96 | 7.607 | 11.963 | 0.090 | 1.00 | 124.64 | A | O |
| ATOM | 601 | N | PHE | A | 97 | 7.022 | 13.388 | −1.560 | 1.00 | 101.47 | A | N |
| ATOM | 602 | CA | PHE | A | 97 | 7.224 | 12.404 | −2.615 | 1.00 | 100.07 | A | C |
| ATOM | 603 | CB | PHE | A | 97 | 6.186 | 12.564 | −3.720 | 1.00 | 99.04 | A | C |
| ATOM | 604 | CG | PHE | A | 97 | 4.818 | 12.146 | −3.291 | 1.00 | 115.62 | A | C |
| ATOM | 605 | CD1 | PHE | A | 97 | 4.057 | 12.987 | −2.502 | 1.00 | 119.44 | A | C |
| ATOM | 606 | CE1 | PHE | A | 97 | 2.799 | 12.612 | −2.074 | 1.00 | 126.86 | A | C |
| ATOM | 607 | CZ | PHE | A | 97 | 2.300 | 11.363 | −2.407 | 1.00 | 123.39 | A | C |
| ATOM | 608 | CE2 | PHE | A | 97 | 3.063 | 10.497 | −3.176 | 1.00 | 115.13 | A | C |
| ATOM | 609 | CD2 | PHE | A | 97 | 4.323 | 10.882 | −3.603 | 1.00 | 113.44 | A | C |
| ATOM | 610 | C | PHE | A | 97 | 8.629 | 12.337 | −3.150 | 1.00 | 98.35 | A | C |
| ATOM | 611 | O | PHE | A | 97 | 8.974 | 11.388 | −3.829 | 1.00 | 103.10 | A | O |
| ATOM | 612 | N | GLY | A | 98 | 9.434 | 13.333 | −2.801 | 1.00 | 113.80 | A | N |
| ATOM | 613 | CA | GLY | A | 98 | 10.865 | 13.338 | −3.115 | 1.00 | 134.88 | A | C |
| ATOM | 614 | C | GLY | A | 98 | 11.053 | 13.559 | −4.595 | 1.00 | 118.48 | A | C |
| ATOM | 615 | O | GLY | A | 98 | 11.549 | 12.688 | −5.308 | 1.00 | 135.04 | A | O |
| ATOM | 616 | N | LEU | A | 99 | 10.663 | 14.740 | −5.053 | 1.00 | 132.80 | A | N |
| ATOM | 617 | CA | LEU | A | 99 | 10.573 | 15.020 | −6.479 | 1.00 | 125.22 | A | C |
| ATOM | 618 | CB | LEU | A | 99 | 9.144 | 15.453 | −6.816 | 1.00 | 127.28 | A | C |
| ATOM | 619 | CG | LEU | A | 99 | 8.008 | 14.470 | −6.535 | 1.00 | 100.76 | A | C |
| ATOM | 620 | CD1 | LEU | A | 99 | 6.694 | 15.213 | −6.404 | 1.00 | 107.73 | A | C |
| ATOM | 621 | CD2 | LEU | A | 99 | 7.873 | 13.437 | −7.623 | 1.00 | 97.95 | A | C |
| ATOM | 622 | C | LEU | A | 99 | 11.583 | 16.085 | −6.940 | 1.00 | 130.98 | A | C |
| ATOM | 623 | O | LEU | A | 99 | 12.341 | 16.632 | −6.138 | 1.00 | 157.55 | A | O |
| ATOM | 624 | N | ASP | A | 100 | 11.578 | 16.369 | −8.241 | 1.00 | 157.54 | A | N |
| ATOM | 625 | CA | ASP | A | 100 | 12.470 | 17.354 | −8.842 | 1.00 | 161.45 | A | C |
| ATOM | 626 | CB | ASP | A | 100 | 12.579 | 17.100 | −10.346 | 1.00 | 172.31 | A | C |
| ATOM | 627 | CG | ASP | A | 100 | 14.007 | 17.077 | −10.839 | 1.00 | 187.34 | A | C |
| ATOM | 628 | OD1 | ASP | A | 100 | 14.810 | 17.938 | −10.408 | 1.00 | 187.23 | A | O |
| ATOM | 629 | OD2 | ASP | A | 100 | 14.318 | 16.189 | −11.666 | 1.00 | 180.28 | A | O |
| ATOM | 630 | C | ASP | A | 100 | 11.990 | 18.780 | −8.596 | 1.00 | 168.55 | A | C |
| ATOM | 631 | O | ASP | A | 100 | 10.819 | 19.127 | −8.823 | 1.00 | 154.54 | A | O |
| ATOM | 632 | N | TRP | A | 101 | 12.918 | 19.615 | −8.153 | 1.00 | 149.23 | A | N |
| ATOM | 633 | CA | TRP | A | 101 | 12.592 | 20.990 | −7.822 | 1.00 | 164.01 | A | C |
| ATOM | 634 | CB | TRP | A | 101 | 13.490 | 21.475 | −6.693 | 1.00 | 164.48 | A | C |
| ATOM | 635 | CG | TRP | A | 101 | 13.180 | 20.818 | −5.390 | 1.00 | 178.62 | A | C |
| ATOM | 636 | CD1 | TRP | A | 101 | 13.875 | 19.782 | −4.778 | 1.00 | 216.55 | A | C |
| ATOM | 637 | NE1 | TRP | A | 101 | 13.280 | 19.433 | −3.594 | 1.00 | 270.41 | A | N |
| ATOM | 638 | CE2 | TRP | A | 101 | 12.171 | 20.169 | −3.380 | 1.00 | 117.61 | A | C |
| ATOM | 639 | CD2 | TRP | A | 101 | 12.050 | 21.101 | −4.505 | 1.00 | 149.39 | A | C |
| ATOM | 640 | CE3 | TRP | A | 101 | 10.995 | 22.002 | −4.526 | 1.00 | 143.56 | A | C |
| ATOM | 641 | CZ3 | TRP | A | 101 | 10.083 | 21.991 | −3.462 | 1.00 | 155.47 | A | C |
| ATOM | 642 | CH2 | TRP | A | 101 | 10.216 | 21.091 | −2.384 | 1.00 | 145.06 | A | C |
| ATOM | 643 | CZ2 | TRP | A | 101 | 11.266 | 20.166 | −2.322 | 1.00 | 160.85 | A | C |
| ATOM | 644 | C | TRP | A | 101 | 12.670 | 21.932 | −8.996 | 1.00 | 165.00 | A | C |
| ATOM | 645 | O | TRP | A | 101 | 12.591 | 23.152 | −8.823 | 1.00 | 160.47 | A | O |
| ATOM | 646 | N | ARG | A | 102 | 12.820 | 21.387 | −10.200 | 1.00 | 143.00 | A | N |
| ATOM | 647 | CA | ARG | A | 102 | 12.898 | 22.208 | −11.396 | 1.00 | 142.94 | A | C |
| ATOM | 648 | CB | ARG | A | 102 | 13.428 | 21.396 | −12.583 | 1.00 | 151.31 | A | C |
| ATOM | 649 | CG | ARG | A | 102 | 14.942 | 21.330 | −12.661 | 1.00 | 169.29 | A | C |
| ATOM | 650 | CD | ARG | A | 102 | 15.430 | 19.915 | −12.942 | 1.00 | 172.38 | A | C |
| ATOM | 651 | NE | ARG | A | 102 | 15.408 | 19.540 | −14.359 | 1.00 | 207.19 | A | N |
| ATOM | 652 | CZ | ARG | A | 102 | 15.448 | 18.286 | −14.817 | 1.00 | 198.92 | A | C |
| ATOM | 653 | NH1 | ARG | A | 102 | 15.480 | 17.256 | −13.981 | 1.00 | 217.80 | A | N |
| ATOM | 654 | NH2 | ARG | A | 102 | 15.447 | 18.055 | −16.126 | 1.00 | 201.69 | A | N |
| ATOM | 655 | C | ARG | A | 102 | 11.528 | 22.822 | −11.692 | 1.00 | 149.41 | A | C |
| ATOM | 656 | O | ARG | A | 102 | 11.379 | 24.051 | −11.647 | 1.00 | 158.67 | A | O |
| ATOM | 657 | N | PRO | A | 103 | 10.518 | 21.975 | −11.969 | 1.00 | 135.34 | A | N |
| ATOM | 658 | CA | PRO | A | 103 | 9.195 | 22.486 | −12.275 | 1.00 | 148.33 | A | C |
| ATOM | 659 | CB | PRO | A | 103 | 8.368 | 21.208 | −12.413 | 1.00 | 135.64 | A | C |
| ATOM | 660 | CG | PRO | A | 103 | 9.335 | 20.214 | −12.933 | 1.00 | 127.64 | A | C |
| ATOM | 661 | CD | PRO | A | 103 | 10.567 | 20.509 | −12.132 | 1.00 | 151.30 | A | C |
| ATOM | 662 | C | PRO | A | 103 | 8.652 | 23.384 | −11.168 | 1.00 | 143.53 | A | C |
| ATOM | 663 | O | PRO | A | 103 | 7.907 | 24.318 | −11.444 | 1.00 | 140.04 | A | O |
| ATOM | 664 | N | TYR | A | 104 | 9.034 | 23.109 | −9.928 | 1.00 | 138.87 | A | N |
| ATOM | 665 | CA | TYR | A | 104 | 8.732 | 24.024 | −8.851 | 1.00 | 141.23 | A | C |
| ATOM | 666 | CB | TYR | A | 104 | 9.169 | 23.439 | −7.518 | 1.00 | 131.96 | A | C |
| ATOM | 667 | CG | TYR | A | 104 | 8.978 | 24.389 | −6.355 | 1.00 | 141.50 | A | C |
| ATOM | 668 | CD1 | TYR | A | 104 | 7.744 | 24.489 | −5.712 | 1.00 | 128.47 | A | C |
| ATOM | 669 | CE1 | TYR | A | 104 | 7.564 | 25.352 | −4.645 | 1.00 | 112.66 | A | C |
| ATOM | 670 | CZ | TYR | A | 104 | 8.619 | 26.131 | −4.198 | 1.00 | 154.25 | A | C |
| ATOM | 671 | OH | TYR | A | 104 | 8.411 | 26.983 | −3.133 | 1.00 | 176.37 | A | O |
| ATOM | 672 | CE2 | TYR | A | 104 | 9.861 | 26.050 | −4.818 | 1.00 | 167.08 | A | C |
| ATOM | 673 | CD2 | TYR | A | 104 | 10.030 | 25.191 | −5.898 | 1.00 | 137.53 | A | C |
| ATOM | 674 | C | TYR | A | 104 | 9.419 | 25.371 | −9.044 | 1.00 | 151.49 | A | C |
| ATOM | 675 | O | TYR | A | 104 | 8.802 | 26.419 | −8.854 | 1.00 | 161.17 | A | O |
| ATOM | 676 | N | SER | A | 105 | 10.704 | 25.341 | −9.390 | 1.00 | 130.68 | A | N |
| ATOM | 677 | CA | SER | A | 105 | 11.503 | 26.561 | −9.420 | 1.00 | 137.72 | A | C |
| ATOM | 678 | CB | SER | A | 105 | 12.985 | 26.233 | −9.480 | 1.00 | 177.91 | A | C |
| ATOM | 679 | OG | SER | A | 105 | 13.273 | 25.427 | −10.613 | 1.00 | 167.44 | A | O |
| ATOM | 680 | C | SER | A | 105 | 11.126 | 27.456 | −10.590 | 1.00 | 159.30 | A | C |
| ATOM | 681 | O | SER | A | 105 | 11.107 | 28.684 | −10.448 | 1.00 | 177.52 | A | O |
| ATOM | 682 | N | TRP | A | 106 | 10.831 | 26.845 | −11.740 | 1.00 | 153.44 | A | N |
| ATOM | 683 | CA | TRP | A | 106 | 10.252 | 27.585 | −12.862 | 1.00 | 158.71 | A | C |
| ATOM | 684 | CB | TRP | A | 106 | 9.955 | 26.672 | −14.047 | 1.00 | 128.25 | A | C |
| ATOM | 685 | CG | TRP | A | 106 | 11.060 | 26.567 | −15.078 | 1.00 | 142.09 | A | C |
| ATOM | 686 | CD1 | TRP | A | 106 | 11.579 | 25.401 | −15.629 | 1.00 | 163.60 | A | C |
| ATOM | 687 | NE1 | TRP | A | 106 | 12.562 | 25.682 | −16.551 | 1.00 | 177.00 | A | N |
| ATOM | 688 | CE2 | TRP | A | 106 | 12.759 | 27.007 | −16.666 | 1.00 | 171.18 | A | C |
| ATOM | 689 | CD2 | TRP | A | 106 | 11.815 | 27.659 | −15.741 | 1.00 | 150.67 | A | C |
| ATOM | 690 | CE3 | TRP | A | 106 | 11.805 | 29.045 | −15.666 | 1.00 | 145.25 | A | C |
| ATOM | 691 | CZ3 | TRP | A | 106 | 12.710 | 29.766 | −16.476 | 1.00 | 194.23 | A | C |
| ATOM | 692 | CH2 | TRP | A | 106 | 13.613 | 29.121 | −17.345 | 1.00 | 203.05 | A | C |
| ATOM | 693 | CZ2 | TRP | A | 106 | 13.651 | 27.735 | −17.464 | 1.00 | 160.73 | A | C |
| ATOM | 694 | C | TRP | A | 106 | 8.992 | 28.284 | −12.407 | 1.00 | 157.63 | A | C |
| ATOM | 695 | O | TRP | A | 106 | 8.860 | 29.496 | −12.572 | 1.00 | 176.56 | A | O |
| ATOM | 696 | N | TYR | A | 107 | 8.065 | 27.532 | −11.811 | 1.00 | 129.97 | A | N |
| ATOM | 697 | CA | TYR | A | 107 | 6.848 | 28.108 | −11.234 | 1.00 | 141.51 | A | C |
| ATOM | 698 | CB | TYR | A | 107 | 6.075 | 27.039 | −10.447 | 1.00 | 142.08 | A | C |
| ATOM | 699 | CG | TYR | A | 107 | 4.809 | 27.535 | −9.743 | 1.00 | 177.22 | A | C |
| ATOM | 700 | CD1 | TYR | A | 107 | 3.599 | 27.668 | −10.439 | 1.00 | 141.37 | A | C |
| ATOM | 701 | CE1 | TYR | A | 107 | 2.446 | 28.110 | −9.801 | 1.00 | 149.88 | A | C |
| ATOM | 702 | CZ | TYR | A | 107 | 2.489 | 28.424 | −8.445 | 1.00 | 156.43 | A | C |
| ATOM | 703 | OH | TYR | A | 107 | 1.357 | 28.874 | −7.808 | 1.00 | 145.52 | A | O |
| ATOM | 704 | CE2 | TYR | A | 107 | 3.670 | 28.298 | −7.733 | 1.00 | 145.99 | A | C |
| ATOM | 705 | CD2 | TYR | A | 107 | 4.818 | 27.856 | −8.376 | 1.00 | 154.08 | A | C |
| ATOM | 706 | C | TYR | A | 107 | 7.143 | 29.328 | −10.351 | 1.00 | 147.46 | A | C |
| ATOM | 707 | O | TYR | A | 107 | 6.490 | 30.365 | −10.471 | 1.00 | 156.83 | A | O |
| ATOM | 708 | N | SER | A | 108 | 8.136 | 29.204 | −9.479 | 1.00 | 154.28 | A | N |
| ATOM | 709 | CA | SER | A | 108 | 8.488 | 30.284 | −8.563 | 1.00 | 171.81 | A | C |
| ATOM | 710 | CB | SER | A | 108 | 9.627 | 29.845 | −7.637 | 1.00 | 185.68 | A | C |
| ATOM | 711 | OG | SER | A | 108 | 9.158 | 28.972 | −6.618 | 1.00 | 184.78 | A | O |
| ATOM | 712 | C | SER | A | 108 | 8.835 | 31.588 | −9.299 | 1.00 | 175.66 | A | C |
| ATOM | 713 | O | SER | A | 108 | 8.471 | 32.682 | −8.835 | 1.00 | 171.99 | A | O |
| ATOM | 714 | N | LEU | A | 109 | 9.528 | 31.456 | −10.439 | 1.00 | 187.03 | A | N |
| ATOM | 715 | CA | LEU | A | 109 | 9.823 | 32.591 | −11.335 | 1.00 | 197.52 | A | C |
| ATOM | 716 | CB | LEU | A | 109 | 10.747 | 32.175 | −12.496 | 1.00 | 195.09 | A | C |
| ATOM | 717 | CG | LEU | A | 109 | 10.738 | 33.083 | −13.736 | 1.00 | 172.37 | A | C |
| ATOM | 718 | CD1 | LEU | A | 109 | 11.787 | 34.176 | −13.606 | 1.00 | 155.35 | A | C |
| ATOM | 719 | CD2 | LEU | A | 109 | 10.905 | 32.284 | −15.022 | 1.00 | 187.61 | A | C |
| ATOM | 720 | C | LEU | A | 109 | 8.540 | 33.193 | −11.904 | 1.00 | 180.50 | A | C |
| ATOM | 721 | O | LEU | A | 109 | 8.301 | 34.399 | −11.785 | 1.00 | 173.92 | A | O |
| ATOM | 722 | N | PHE | A | 110 | 7.737 | 32.346 | −12.540 | 1.00 | 163.63 | A | N |
| ATOM | 723 | CA | PHE | A | 110 | 6.453 | 32.768 | −13.044 | 1.00 | 155.30 | A | C |
| ATOM | 724 | CB | PHE | A | 110 | 5.604 | 31.559 | −13.440 | 1.00 | 176.97 | A | C |
| ATOM | 725 | CG | PHE | A | 110 | 4.171 | 31.911 | −13.743 | 1.00 | 182.19 | A | C |
| ATOM | 726 | CD1 | PHE | A | 110 | 3.276 | 32.211 | −12.712 | 1.00 | 187.24 | A | C |
| ATOM | 727 | CE1 | PHE | A | 110 | 1.962 | 32.552 | −12.987 | 1.00 | 185.42 | A | C |
| ATOM | 728 | CZ | PHE | A | 110 | 1.522 | 32.593 | −14.301 | 1.00 | 187.73 | A | C |
| ATOM | 729 | CE2 | PHE | A | 110 | 2.395 | 32.284 | −15.336 | 1.00 | 198.69 | A | C |
| ATOM | 730 | CD2 | PHE | A | 110 | 3.714 | 31.960 | −15.056 | 1.00 | 187.73 | A | C |
| ATOM | 731 | C | PHE | A | 110 | 5.708 | 33.614 | −12.005 | 1.00 | 153.42 | A | C |
| ATOM | 732 | O | PHE | A | 110 | 5.164 | 34.675 | −12.334 | 1.00 | 152.40 | A | O |
| ATOM | 733 | N | VAL | A | 111 | 5.680 | 33.141 | −10.762 | 1.00 | 142.26 | A | N |
| ATOM | 734 | CA | VAL | A | 111 | 5.026 | 33.875 | −9.685 | 1.00 | 148.17 | A | C |
| ATOM | 735 | CB | VAL | A | 111 | 5.121 | 33.136 | −8.338 | 1.00 | 170.59 | A | C |
| ATOM | 736 | CG1 | VAL | A | 111 | 4.673 | 34.039 | −7.177 | 1.00 | 146.31 | A | C |
| ATOM | 737 | CG2 | VAL | A | 111 | 4.264 | 31.884 | −8.393 | 1.00 | 177.64 | A | C |
| ATOM | 738 | C | VAL | A | 111 | 5.595 | 35.277 | −9.549 | 1.00 | 162.92 | A | C |
| ATOM | 739 | O | VAL | A | 111 | 4.848 | 36.260 | −9.582 | 1.00 | 167.11 | A | O |
| ATOM | 740 | N | ALA | A | 112 | 6.914 | 35.367 | −9.414 | 1.00 | 158.16 | A | N |
| ATOM | 741 | CA | ALA | A | 112 | 7.581 | 36.661 | −9.332 | 1.00 | 166.78 | A | C |
| ATOM | 742 | CB | ALA | A | 112 | 9.080 | 36.469 | −9.225 | 1.00 | 161.25 | A | C |
| ATOM | 743 | C | ALA | A | 112 | 7.230 | 37.571 | −10.520 | 1.00 | 180.83 | A | C |
| ATOM | 744 | O | ALA | A | 112 | 6.941 | 38.754 | −10.332 | 1.00 | 176.48 | A | O |
| ATOM | 745 | N | ILE | A | 113 | 7.231 | 37.007 | −11.730 | 1.00 | 178.48 | A | N |
| ATOM | 746 | CA | ILE | A | 113 | 6.850 | 37.746 | −12.942 | 1.00 | 185.46 | A | C |
| ATOM | 747 | CB | ILE | A | 113 | 6.878 | 36.847 | −14.206 | 1.00 | 174.16 | A | C |
| ATOM | 748 | CG1 | ILE | A | 113 | 8.279 | 36.267 | −14.430 | 1.00 | 151.93 | A | C |
| ATOM | 749 | CD1 | ILE | A | 113 | 8.382 | 35.339 | −15.621 | 1.00 | 150.13 | A | C |
| ATOM | 750 | CG2 | ILE | A | 113 | 6.440 | 37.624 | −15.441 | 1.00 | 163.23 | A | C |
| ATOM | 751 | C | ILE | A | 113 | 5.468 | 38.395 | −12.782 | 1.00 | 169.36 | A | C |
| ATOM | 752 | O | ILE | A | 113 | 5.192 | 39.442 | −13.354 | 1.00 | 158.63 | A | O |
| ATOM | 753 | N | ASN | A | 114 | 4.603 | 37.780 | −11.989 | 1.00 | 165.89 | A | N |
| ATOM | 754 | CA | ASN | A | 114 | 3.272 | 38.324 | −11.784 | 1.00 | 167.15 | A | C |
| ATOM | 755 | CB | ASN | A | 114 | 2.210 | 37.224 | −11.872 | 1.00 | 172.05 | A | C |
| ATOM | 756 | CG | ASN | A | 114 | 2.207 | 36.520 | −13.214 | 1.00 | 153.90 | A | C |
| ATOM | 757 | OD1 | ASN | A | 114 | 1.150 | 36.141 | −13.725 | 1.00 | 177.61 | A | O |
| ATOM | 758 | ND2 | ASN | A | 114 | 3.392 | 36.337 | −13.791 | 1.00 | 165.28 | A | N |
| ATOM | 759 | C | ASN | A | 114 | 3.137 | 39.103 | −10.482 | 1.00 | 169.27 | A | C |
| ATOM | 760 | O | ASN | A | 114 | 2.068 | 39.619 | −10.184 | 1.00 | 196.72 | A | O |
| ATOM | 761 | N | THR | A | 115 | 4.218 | 39.184 | −9.711 | 1.00 | 178.64 | A | N |
| ATOM | 762 | CA | THR | A | 115 | 4.232 | 40.025 | −8.515 | 1.00 | 164.58 | A | C |
| ATOM | 763 | CB | THR | A | 115 | 5.211 | 39.507 | −7.457 | 1.00 | 188.26 | A | C |
| ATOM | 764 | OG1 | THR | A | 115 | 6.555 | 39.653 | −7.934 | 1.00 | 206.86 | A | O |
| ATOM | 765 | CG2 | THR | A | 115 | 4.929 | 38.051 | −7.138 | 1.00 | 194.55 | A | C |
| ATOM | 766 | C | THR | A | 115 | 4.603 | 41.462 | −8.857 | 1.00 | 185.11 | A | C |
| ATOM | 767 | O | THR | A | 115 | 4.346 | 42.386 | −8.076 | 1.00 | 209.89 | A | O |
| ATOM | 768 | N | VAL | A | 116 | 5.231 | 41.634 | −10.019 | 1.00 | 205.56 | A | N |
| ATOM | 769 | CA | VAL | A | 116 | 5.602 | 42.951 | −10.541 | 1.00 | 200.40 | A | C |
| ATOM | 770 | CB | VAL | A | 116 | 6.419 | 42.834 | −11.848 | 1.00 | 185.42 | A | C |
| ATOM | 771 | CG1 | VAL | A | 116 | 6.587 | 44.189 | −12.501 | 1.00 | 164.56 | A | C |
| ATOM | 772 | CG2 | VAL | A | 116 | 7.770 | 42.197 | −11.576 | 1.00 | 157.66 | A | C |
| ATOM | 773 | C | VAL | A | 116 | 4.345 | 43.784 | −10.783 | 1.00 | 193.06 | A | C |
| ATOM | 774 | O | VAL | A | 116 | 4.196 | 44.865 | −10.191 | 1.00 | 199.11 | A | O |
| ATOM | 775 | N | PRO | A | 117 | 3.432 | 43.285 | −11.653 | 1.00 | 201.76 | A | N |
| ATOM | 776 | CA | PRO | A | 117 | 2.197 | 44.015 | −11.929 | 1.00 | 196.10 | A | C |
| ATOM | 777 | CB | PRO | A | 117 | 1.546 | 43.207 | −13.058 | 1.00 | 184.55 | A | C |
| ATOM | 778 | CG | PRO | A | 117 | 2.613 | 42.324 | −13.613 | 1.00 | 184.87 | A | C |
| ATOM | 779 | CD | PRO | A | 117 | 3.531 | 42.057 | −12.466 | 1.00 | 188.01 | A | C |
| ATOM | 780 | C | PRO | A | 117 | 1.298 | 44.066 | −10.693 | 1.00 | 184.10 | A | C |
| ATOM | 781 | O | PRO | A | 117 | 0.494 | 44.990 | −10.539 | 1.00 | 169.78 | A | O |
| ATOM | 782 | N | ALA | A | 118 | 1.451 | 43.090 | −9.809 | 1.00 | 197.38 | A | N |
| ATOM | 783 | CA | ALA | A | 118 | 0.746 | 43.099 | −8.533 | 1.00 | 204.52 | A | C |
| ATOM | 784 | CB | ALA | A | 118 | 0.986 | 41.803 | −7.785 | 1.00 | 202.57 | A | C |
| ATOM | 785 | C | ALA | A | 118 | 1.172 | 44.288 | −7.689 | 1.00 | 189.93 | A | C |
| ATOM | 786 | O | ALA | A | 118 | 0.330 | 44.971 | −7.113 | 1.00 | 209.06 | A | O |
| ATOM | 787 | N | ALA | A | 119 | 2.475 | 44.541 | −7.643 | 1.00 | 160.78 | A | N |
| ATOM | 788 | CA | ALA | A | 119 | 3.016 | 45.664 | −6.900 | 1.00 | 173.35 | A | C |
| ATOM | 789 | CB | ALA | A | 119 | 4.529 | 45.591 | −6.888 | 1.00 | 180.67 | A | C |
| ATOM | 790 | C | ALA | A | 119 | 2.534 | 47.000 | −7.480 | 1.00 | 203.29 | A | C |
| ATOM | 791 | O | ALA | A | 119 | 1.993 | 47.839 | −6.745 | 1.00 | 178.53 | A | O |
| ATOM | 792 | N | ILE | A | 120 | 2.730 | 47.177 | −8.794 | 1.00 | 219.18 | A | N |
| ATOM | 793 | CA | ILE | A | 120 | 2.202 | 48.322 | −9.552 | 1.00 | 207.35 | A | C |
| ATOM | 794 | CB | ILE | A | 120 | 2.299 | 48.094 | −11.091 | 1.00 | 182.60 | A | C |
| ATOM | 795 | CG1 | ILE | A | 120 | 3.753 | 48.182 | −11.562 | 1.00 | 175.01 | A | C |
| ATOM | 796 | CD1 | ILE | A | 120 | 3.990 | 47.662 | −12.969 | 1.00 | 182.37 | A | C |
| ATOM | 797 | CG2 | ILE | A | 120 | 1.432 | 49.086 | −11.865 | 1.00 | 185.88 | A | C |
| ATOM | 798 | C | ILE | A | 120 | 0.750 | 48.607 | −9.150 | 1.00 | 218.98 | A | C |
| ATOM | 799 | O | ILE | A | 120 | 0.425 | 49.699 | −8.681 | 1.00 | 208.56 | A | O |
| ATOM | 800 | N | LEU | A | 121 | −0.107 | 47.602 | −9.310 | 1.00 | 202.77 | A | N |
| ATOM | 801 | CA | LEU | A | 121 | −1.536 | 47.766 | −9.109 | 1.00 | 208.55 | A | C |
| ATOM | 802 | CB | LEU | A | 121 | −2.307 | 46.577 | −9.735 | 1.00 | 206.45 | A | C |
| ATOM | 803 | CG | LEU | A | 121 | −3.751 | 46.829 | −10.243 | 1.00 | 250.90 | A | C |
| ATOM | 804 | CD1 | LEU | A | 121 | −4.004 | 48.309 | −10.645 | 1.00 | 182.56 | A | C |
| ATOM | 805 | CD2 | LEU | A | 121 | −4.166 | 45.837 | −11.335 | 1.00 | 267.90 | A | C |
| ATOM | 806 | C | LEU | A | 121 | −1.880 | 47.971 | −7.627 | 1.00 | 212.73 | A | C |
| ATOM | 807 | O | LEU | A | 121 | −2.886 | 48.602 | −7.295 | 1.00 | 173.98 | A | O |
| ATOM | 808 | N | SER | A | 122 | −1.028 | 47.464 | −6.740 | 1.00 | 217.29 | A | N |
| ATOM | 809 | CA | SER | A | 122 | −1.323 | 47.477 | −5.302 | 1.00 | 211.14 | A | C |
| ATOM | 810 | CB | SER | A | 122 | −0.628 | 46.318 | −4.579 | 1.00 | 175.63 | A | C |
| ATOM | 811 | OG | SER | A | 122 | 0.775 | 46.384 | −4.747 | 1.00 | 184.23 | A | O |
| ATOM | 812 | C | SER | A | 122 | −0.963 | 48.798 | −4.648 | 1.00 | 185.26 | A | C |
| ATOM | 813 | O | SER | A | 122 | −1.433 | 49.101 | −3.555 | 1.00 | 195.05 | A | O |
| ATOM | 814 | N | HIS | A | 123 | −0.132 | 49.577 | −5.330 | 1.00 | 207.92 | A | N |
| ATOM | 815 | CA | HIS | A | 123 | 0.236 | 50.894 | −4.848 | 1.00 | 230.56 | A | C |
| ATOM | 816 | CB | HIS | A | 123 | 1.583 | 51.320 | −5.424 | 1.00 | 239.21 | A | C |
| ATOM | 817 | CG | HIS | A | 123 | 2.032 | 52.682 | −4.956 | 1.00 | 241.70 | A | C |
| ATOM | 818 | ND1 | HIS | A | 123 | 2.365 | 52.927 | −3.675 | 1.00 | 223.34 | A | N |
| ATOM | 819 | CE1 | HIS | A | 123 | 2.702 | 54.218 | −3.534 | 1.00 | 234.30 | A | C |
| ATOM | 820 | NE2 | HIS | A | 123 | 2.588 | 54.812 | −4.740 | 1.00 | 259.43 | A | N |
| ATOM | 821 | CD2 | HIS | A | 123 | 2.166 | 53.892 | −5.639 | 1.00 | 252.89 | A | C |
| ATOM | 822 | C | HIS | A | 123 | −0.812 | 51.943 | −5.138 | 1.00 | 254.27 | A | C |
| ATOM | 823 | O | HIS | A | 123 | −0.834 | 52.991 | −4.491 | 1.00 | 261.23 | A | O |
| ATOM | 824 | N | TYR | A | 124 | −1.686 | 51.675 | −6.108 | 1.00 | 242.24 | A | N |
| ATOM | 825 | CA | TYR | A | 124 | −2.722 | 52.633 | −6.511 | 1.00 | 236.62 | A | C |
| ATOM | 826 | CB | TYR | A | 124 | −2.856 | 52.696 | −8.038 | 1.00 | 217.76 | A | C |
| ATOM | 827 | CG | TYR | A | 124 | −1.574 | 53.002 | −8.777 | 1.00 | 243.43 | A | C |
| ATOM | 828 | CD1 | TYR | A | 124 | −0.549 | 53.747 | −8.169 | 1.00 | 241.70 | A | C |
| ATOM | 829 | CE1 | TYR | A | 124 | 0.625 | 54.037 | −8.845 | 1.00 | 239.37 | A | C |
| ATOM | 830 | CZ | TYR | A | 124 | 0.779 | 53.596 | −10.156 | 1.00 | 254.60 | A | C |
| ATOM | 831 | OH | TYR | A | 124 | 1.947 | 53.881 | −10.828 | 1.00 | 273.95 | A | O |
| ATOM | 832 | CE2 | TYR | A | 124 | −0.225 | 52.867 | −10.785 | 1.00 | 246.05 | A | C |
| ATOM | 833 | CD2 | TYR | A | 124 | −1.394 | 52.568 | −10.095 | 1.00 | 230.37 | A | C |
| ATOM | 834 | C | TYR | A | 124 | −4.080 | 52.314 | −5.886 | 1.00 | 245.41 | A | C |
| ATOM | 835 | O | TYR | A | 124 | −5.129 | 52.457 | −6.529 | 1.00 | 265.01 | A | O |
| ATOM | 836 | N | SER | A | 125 | −4.054 | 51.873 | −4.633 | 1.00 | 231.09 | A | N |
| ATOM | 837 | CA | SER | A | 125 | −5.272 | 51.523 | −3.910 | 1.00 | 212.56 | A | C |
| ATOM | 838 | CB | SER | A | 125 | −5.513 | 49.999 | −3.963 | 1.00 | 275.48 | A | C |
| ATOM | 839 | OG | SER | A | 125 | −5.883 | 49.531 | −5.262 | 1.00 | 250.64 | A | O |
| ATOM | 840 | C | SER | A | 125 | −5.166 | 51.968 | −2.448 | 1.00 | 258.95 | A | C |
| ATOM | 841 | O | SER | A | 125 | −4.077 | 52.289 | −1.955 | 1.00 | 242.30 | A | O |
| ATOM | 842 | N | ASP | A | 126 | −6.313 | 51.996 | −1.772 | 1.00 | 294.48 | A | N |
| ATOM | 843 | CA | ASP | A | 126 | −6.372 | 51.933 | −0.309 | 1.00 | 286.03 | A | C |
| ATOM | 844 | CB | ASP | A | 126 | −6.023 | 50.502 | 0.173 | 1.00 | 305.46 | A | C |
| ATOM | 845 | CG | ASP | A | 126 | −6.933 | 49.415 | −0.407 | 1.00 | 326.54 | A | C |
| ATOM | 846 | OD1 | ASP | A | 126 | −8.023 | 49.744 | −0.925 | 1.00 | 327.63 | A | O |
| ATOM | 847 | OD2 | ASP | A | 126 | −6.545 | 48.223 | −0.331 | 1.00 | 274.19 | A | O |
| ATOM | 848 | C | ASP | A | 126 | −5.479 | 52.942 | 0.435 | 1.00 | 250.05 | A | C |
| ATOM | 849 | O | ASP | A | 126 | −4.759 | 52.558 | 1.356 | 1.00 | 260.55 | A | O |
| ATOM | 850 | N | MET | A | 127 | −5.504 | 54.216 | 0.038 | 1.00 | 215.42 | A | N |
| ATOM | 851 | CA | MET | A | 127 | −4.876 | 55.272 | 0.853 | 1.00 | 239.53 | A | C |
| ATOM | 852 | CB | MET | A | 127 | −4.535 | 56.497 | 0.007 | 1.00 | 224.51 | A | C |
| ATOM | 853 | CG | MET | A | 127 | −3.574 | 56.178 | −1.131 | 1.00 | 249.46 | A | C |
| ATOM | 854 | SD | MET | A | 127 | −4.023 | 57.038 | −2.648 | 1.00 | 315.42 | A | S |
| ATOM | 855 | CE | MET | A | 127 | −3.505 | 55.896 | −3.927 | 1.00 | 280.28 | A | C |
| ATOM | 856 | C | MET | A | 127 | −5.806 | 55.625 | 2.020 | 1.00 | 237.48 | A | C |
| ATOM | 857 | O | MET | A | 127 | −6.840 | 56.274 | 1.844 | 1.00 | 247.35 | A | O |
| ATOM | 858 | N | LEU | A | 128 | −5.427 | 55.186 | 3.217 | 1.00 | 260.19 | A | N |
| ATOM | 859 | CA | LEU | A | 128 | −6.386 | 55.059 | 4.310 | 1.00 | 241.30 | A | C |
| ATOM | 860 | CB | LEU | A | 128 | −6.271 | 53.672 | 4.964 | 1.00 | 248.50 | A | C |
| ATOM | 861 | CG | LEU | A | 128 | −6.508 | 52.487 | 4.022 | 1.00 | 205.82 | A | C |
| ATOM | 862 | CD1 | LEU | A | 128 | −6.286 | 51.166 | 4.729 | 1.00 | 198.91 | A | C |
| ATOM | 863 | CD2 | LEU | A | 128 | −7.882 | 52.552 | 3.364 | 1.00 | 169.47 | A | C |
| ATOM | 864 | C | LEU | A | 128 | −6.318 | 56.175 | 5.339 | 1.00 | 237.00 | A | C |
| ATOM | 865 | O | LEU | A | 128 | −7.355 | 56.659 | 5.804 | 1.00 | 237.62 | A | O |
| ATOM | 866 | N | ASP | A | 129 | −5.104 | 56.600 | 5.674 | 1.00 | 238.31 | A | N |
| ATOM | 867 | CA | ASP | A | 129 | −4.902 | 57.784 | 6.516 | 1.00 | 276.57 | A | C |
| ATOM | 868 | CB | ASP | A | 129 | −5.804 | 58.960 | 6.063 | 1.00 | 322.48 | A | C |
| ATOM | 869 | CG | ASP | A | 129 | −5.538 | 59.432 | 4.612 | 1.00 | 299.56 | A | C |
| ATOM | 870 | OD1 | ASP | A | 129 | −4.485 | 59.111 | 4.020 | 1.00 | 253.39 | A | O |
| ATOM | 871 | OD2 | ASP | A | 129 | −6.406 | 60.150 | 4.059 | 1.00 | 298.88 | A | O |
| ATOM | 872 | C | ASP | A | 129 | −5.171 | 57.518 | 8.009 | 1.00 | 298.12 | A | C |
| ATOM | 873 | O | ASP | A | 129 | −5.027 | 58.422 | 8.834 | 1.00 | 318.07 | A | O |
| ATOM | 874 | N | ASP | A | 130 | −5.551 | 56.292 | 8.358 | 1.00 | 297.19 | A | N |
| ATOM | 875 | CA | ASP | A | 130 | −6.157 | 56.027 | 9.664 | 1.00 | 324.63 | A | C |
| ATOM | 876 | CB | ASP | A | 130 | −7.571 | 55.463 | 9.469 | 1.00 | 338.72 | A | C |
| ATOM | 877 | CG | ASP | A | 130 | −8.583 | 56.536 | 9.048 | 1.00 | 343.38 | A | C |
| ATOM | 878 | OD1 | ASP | A | 130 | −8.247 | 57.745 | 9.077 | 1.00 | 289.71 | A | O |
| ATOM | 879 | OD2 | ASP | A | 130 | −9.731 | 56.167 | 8.697 | 1.00 | 350.35 | A | O |
| ATOM | 880 | C | ASP | A | 130 | −5.357 | 55.116 | 10.585 | 1.00 | 331.43 | A | C |
| ATOM | 881 | O | ASP | A | 130 | −5.874 | 54.626 | 11.588 | 1.00 | 293.06 | A | O |
| ATOM | 882 | N | HIS | A | 131 | −4.085 | 54.914 | 10.266 | 1.00 | 326.74 | A | N |
| ATOM | 883 | CA | HIS | A | 131 | −3.291 | 53.869 | 10.913 | 1.00 | 301.88 | A | C |
| ATOM | 884 | CB | HIS | A | 131 | −2.478 | 53.097 | 9.874 | 1.00 | 283.81 | A | C |
| ATOM | 885 | CG | HIS | A | 131 | −3.316 | 52.462 | 8.792 | 1.00 | 245.11 | A | C |
| ATOM | 886 | ND1 | HIS | A | 131 | −4.352 | 53.099 | 8.208 | 1.00 | 255.69 | A | N |
| ATOM | 887 | CE1 | HIS | A | 131 | −4.915 | 52.292 | 7.300 | 1.00 | 249.40 | A | C |
| ATOM | 888 | NE2 | HIS | A | 131 | −4.239 | 51.143 | 7.290 | 1.00 | 263.10 | A | N |
| ATOM | 889 | CD2 | HIS | A | 131 | −3.246 | 51.213 | 8.203 | 1.00 | 244.39 | A | C |
| ATOM | 890 | C | HIS | A | 131 | −2.378 | 54.387 | 11.981 | 1.00 | 314.43 | A | C |
| ATOM | 891 | O | HIS | A | 131 | −1.871 | 55.507 | 11.892 | 1.00 | 310.05 | A | O |
| ATOM | 892 | N | LYS | A | 132 | −2.163 | 53.569 | 13.009 | 1.00 | 322.43 | A | N |
| ATOM | 893 | CA | LYS | A | 132 | −1.051 | 53.776 | 13.925 | 1.00 | 332.38 | A | C |
| ATOM | 894 | CB | LYS | A | 132 | −1.332 | 53.190 | 15.323 | 1.00 | 277.63 | A | C |
| ATOM | 895 | CG | LYS | A | 132 | −1.847 | 54.226 | 16.323 | 1.00 | 268.06 | A | C |
| ATOM | 896 | CD | LYS | A | 132 | −2.090 | 53.641 | 17.715 | 1.00 | 266.67 | A | C |
| ATOM | 897 | CE | LYS | A | 132 | −1.646 | 54.579 | 18.844 | 1.00 | 304.19 | A | C |
| ATOM | 898 | NZ | LYS | A | 132 | −2.224 | 55.963 | 18.875 | 1.00 | 312.32 | A | N |
| ATOM | 899 | C | LYS | A | 132 | 0.237 | 53.237 | 13.274 | 1.00 | 335.69 | A | C |
| ATOM | 900 | O | LYS | A | 132 | 1.323 | 53.329 | 13.855 | 1.00 | 352.69 | A | O |
| ATOM | 901 | N | VAL | A | 133 | 0.112 | 52.705 | 12.053 | 1.00 | 305.28 | A | N |
| ATOM | 902 | CA | VAL | A | 133 | 1.279 | 52.436 | 11.204 | 1.00 | 288.41 | A | C |
| ATOM | 903 | CB | VAL | A | 133 | 0.887 | 51.947 | 9.784 | 1.00 | 231.72 | A | C |
| ATOM | 904 | CG1 | VAL | A | 133 | 0.765 | 53.102 | 8.795 | 1.00 | 274.13 | A | C |
| ATOM | 905 | CG2 | VAL | A | 133 | 1.900 | 50.938 | 9.286 | 1.00 | 224.99 | A | C |
| ATOM | 906 | C | VAL | A | 133 | 2.184 | 53.684 | 11.196 | 1.00 | 279.68 | A | C |
| ATOM | 907 | O | VAL | A | 133 | 1.720 | 54.801 | 11.412 | 1.00 | 282.46 | A | O |
| ATOM | 908 | N | LEU | A | 134 | 3.472 | 53.484 | 10.950 | 1.00 | 268.94 | A | N |
| ATOM | 909 | CA | LEU | A | 134 | 4.509 | 54.414 | 11.419 | 1.00 | 240.41 | A | C |
| ATOM | 910 | CB | LEU | A | 134 | 5.888 | 53.761 | 11.343 | 1.00 | 251.61 | A | C |
| ATOM | 911 | CG | LEU | A | 134 | 6.121 | 52.521 | 12.209 | 1.00 | 273.17 | A | C |
| ATOM | 912 | CD1 | LEU | A | 134 | 5.214 | 51.350 | 11.841 | 1.00 | 241.61 | A | C |
| ATOM | 913 | CD2 | LEU | A | 134 | 7.580 | 52.107 | 12.115 | 1.00 | 278.15 | A | C |
| ATOM | 914 | C | LEU | A | 134 | 4.536 | 55.798 | 10.773 | 1.00 | 284.22 | A | C |
| ATOM | 915 | O | LEU | A | 134 | 3.848 | 56.047 | 9.777 | 1.00 | 288.07 | A | O |
| ATOM | 916 | N | GLY | A | 135 | 5.349 | 56.683 | 11.360 | 1.00 | 307.18 | A | N |
| ATOM | 917 | CA | GLY | A | 135 | 5.382 | 58.121 | 11.044 | 1.00 | 289.21 | A | C |
| ATOM | 918 | C | GLY | A | 135 | 6.196 | 58.536 | 9.831 | 1.00 | 319.46 | A | C |
| ATOM | 919 | O | GLY | A | 135 | 6.256 | 59.729 | 9.491 | 1.00 | 400.80 | A | O |
| ATOM | 920 | N | ILE | A | 136 | 6.842 | 57.564 | 9.187 | 1.00 | 376.91 | A | N |
| ATOM | 921 | CA | ILE | A | 136 | 7.456 | 57.805 | 7.882 | 1.00 | 366.80 | A | C |
| ATOM | 922 | CB | ILE | A | 136 | 8.811 | 57.073 | 7.701 | 1.00 | 298.81 | A | C |
| ATOM | 923 | CG1 | ILE | A | 136 | 9.660 | 57.103 | 8.985 | 1.00 | 260.56 | A | C |
| ATOM | 924 | CD1 | ILE | A | 136 | 10.601 | 55.917 | 9.134 | 1.00 | 146.25 | A | C |
| ATOM | 925 | CG2 | ILE | A | 136 | 9.573 | 57.691 | 6.533 | 1.00 | 243.26 | A | C |
| ATOM | 926 | C | ILE | A | 136 | 6.473 | 57.381 | 6.774 | 1.00 | 354.61 | A | C |
| ATOM | 927 | O | ILE | A | 136 | 6.449 | 56.219 | 6.356 | 1.00 | 336.01 | A | O |
| ATOM | 928 | N | THR | A | 137 | 5.660 | 58.330 | 6.307 | 1.00 | 344.27 | A | N |
| ATOM | 929 | CA | THR | A | 137 | 4.675 | 58.093 | 5.224 | 1.00 | 328.80 | A | C |
| ATOM | 930 | CB | THR | A | 137 | 5.299 | 58.290 | 3.817 | 1.00 | 279.86 | A | C |
| ATOM | 931 | OG1 | THR | A | 137 | 6.519 | 57.542 | 3.717 | 1.00 | 300.71 | A | O |
| ATOM | 932 | CG2 | THR | A | 137 | 5.586 | 59.766 | 3.559 | 1.00 | 272.57 | A | C |
| ATOM | 933 | C | THR | A | 137 | 3.896 | 56.754 | 5.329 | 1.00 | 295.08 | A | C |
| ATOM | 934 | O | THR | A | 137 | 3.107 | 56.574 | 6.255 | 1.00 | 268.12 | A | O |
| ATOM | 935 | N | GLU | A | 138 | 4.123 | 55.825 | 4.399 | 1.00 | 261.93 | A | N |
| ATOM | 936 | CA | GLU | A | 138 | 3.465 | 54.502 | 4.416 | 1.00 | 245.12 | A | C |
| ATOM | 937 | CB | GLU | A | 138 | 3.543 | 53.871 | 5.801 | 1.00 | 246.59 | A | C |
| ATOM | 938 | CG | GLU | A | 138 | 4.949 | 53.526 | 6.221 | 1.00 | 228.43 | A | C |
| ATOM | 939 | CD | GLU | A | 138 | 5.095 | 53.517 | 7.720 | 1.00 | 264.30 | A | C |
| ATOM | 940 | OE1 | GLU | A | 138 | 4.443 | 52.673 | 8.365 | 1.00 | 270.37 | A | O |
| ATOM | 941 | OE2 | GLU | A | 138 | 5.857 | 54.351 | 8.250 | 1.00 | 289.84 | A | O |
| ATOM | 942 | C | GLU | A | 138 | 2.012 | 54.504 | 3.924 | 1.00 | 232.58 | A | C |
| ATOM | 943 | O | GLU | A | 138 | 1.150 | 53.795 | 4.463 | 1.00 | 205.83 | A | O |
| ATOM | 944 | N | GLY | A | 139 | 1.753 | 55.309 | 2.904 | 1.00 | 231.52 | A | N |
| ATOM | 945 | CA | GLY | A | 139 | 0.501 | 55.228 | 2.172 | 1.00 | 254.23 | A | C |
| ATOM | 946 | C | GLY | A | 139 | 0.521 | 53.927 | 1.405 | 1.00 | 219.82 | A | C |
| ATOM | 947 | O | GLY | A | 139 | 1.144 | 53.845 | 0.347 | 1.00 | 209.94 | A | O |
| ATOM | 948 | N | ASP | A | 140 | −0.123 | 52.904 | 1.972 | 1.00 | 253.95 | A | N |
| ATOM | 949 | CA | ASP | A | 140 | −0.297 | 51.579 | 1.338 | 1.00 | 252.30 | A | C |
| ATOM | 950 | CB | ASP | A | 140 | −1.446 | 51.610 | 0.324 | 1.00 | 231.60 | A | C |
| ATOM | 951 | CG | ASP | A | 140 | −1.799 | 50.233 | −0.193 | 1.00 | 234.11 | A | C |
| ATOM | 952 | OD1 | ASP | A | 140 | −2.297 | 49.400 | 0.601 | 1.00 | 239.81 | A | O |
| ATOM | 953 | OD2 | ASP | A | 140 | −1.577 | 49.992 | −1.398 | 1.00 | 225.10 | A | O |
| ATOM | 954 | C | ASP | A | 140 | 0.959 | 50.961 | 0.697 | 1.00 | 232.22 | A | C |
| ATOM | 955 | O | ASP | A | 140 | 1.085 | 50.890 | −0.531 | 1.00 | 218.62 | A | O |
| ATOM | 956 | N | TRP | A | 141 | 1.867 | 50.485 | 1.539 | 1.00 | 231.88 | A | N |
| ATOM | 957 | CA | TRP | A | 141 | 3.098 | 49.849 | 1.070 | 1.00 | 213.27 | A | C |
| ATOM | 958 | CB | TRP | A | 141 | 4.253 | 50.112 | 2.035 | 1.00 | 200.96 | A | C |
| ATOM | 959 | CG | TRP | A | 141 | 3.939 | 49.875 | 3.497 | 1.00 | 232.59 | A | C |
| ATOM | 960 | CD1 | TRP | A | 141 | 2.926 | 50.458 | 4.254 | 1.00 | 236.53 | A | C |
| ATOM | 961 | NE1 | TRP | A | 141 | 2.964 | 50.031 | 5.554 | 1.00 | 236.28 | A | N |
| ATOM | 962 | CE2 | TRP | A | 141 | 3.983 | 49.167 | 5.743 | 1.00 | 240.92 | A | C |
| ATOM | 963 | CD2 | TRP | A | 141 | 4.670 | 49.018 | 4.447 | 1.00 | 230.41 | A | C |
| ATOM | 964 | CE3 | TRP | A | 141 | 5.784 | 48.189 | 4.366 | 1.00 | 244.61 | A | C |
| ATOM | 965 | CZ3 | TRP | A | 141 | 6.192 | 47.503 | 5.522 | 1.00 | 297.68 | A | C |
| ATOM | 966 | CH2 | TRP | A | 141 | 5.517 | 47.654 | 6.748 | 1.00 | 315.09 | A | C |
| ATOM | 967 | CZ2 | TRP | A | 141 | 4.403 | 48.492 | 6.882 | 1.00 | 286.85 | A | C |
| ATOM | 968 | C | TRP | A | 141 | 2.962 | 48.383 | 0.743 | 1.00 | 209.93 | A | C |
| ATOM | 969 | O | TRP | A | 141 | 3.966 | 47.679 | 0.584 | 1.00 | 204.25 | A | O |
| ATOM | 970 | N | TRP | A | 142 | 1.718 | 47.914 | 0.623 | 1.00 | 219.90 | A | N |
| ATOM | 971 | CA | TRP | A | 142 | 1.423 | 46.646 | −0.051 | 1.00 | 209.42 | A | C |
| ATOM | 972 | CB | TRP | A | 142 | −0.056 | 46.581 | −0.439 | 1.00 | 233.63 | A | C |
| ATOM | 973 | CG | TRP | A | 142 | −0.922 | 45.733 | 0.469 | 1.00 | 236.48 | A | C |
| ATOM | 974 | CD1 | TRP | A | 142 | −0.947 | 45.731 | 1.869 | 1.00 | 181.85 | A | C |
| ATOM | 975 | NE1 | TRP | A | 142 | −1.868 | 44.828 | 2.341 | 1.00 | 181.39 | A | N |
| ATOM | 976 | CE2 | TRP | A | 142 | −2.491 | 44.200 | 1.312 | 1.00 | 218.44 | A | C |
| ATOM | 977 | CD2 | TRP | A | 142 | −1.935 | 44.740 | 0.066 | 1.00 | 257.17 | A | C |
| ATOM | 978 | CE3 | TRP | A | 142 | −2.416 | 44.255 | −1.164 | 1.00 | 268.57 | A | C |
| ATOM | 979 | CZ3 | TRP | A | 142 | −3.419 | 43.266 | −1.155 | 1.00 | 238.29 | A | C |
| ATOM | 980 | CH2 | TRP | A | 142 | −3.932 | 42.759 | 0.050 | 1.00 | 228.43 | A | C |
| ATOM | 981 | CZ2 | TRP | A | 142 | −3.477 | 43.219 | 1.306 | 1.00 | 217.51 | A | C |
| ATOM | 982 | C | TRP | A | 142 | 2.270 | 46.459 | −1.287 | 1.00 | 205.48 | A | C |
| ATOM | 983 | O | TRP | A | 142 | 2.699 | 45.346 | −1.603 | 1.00 | 195.01 | A | O |
| ATOM | 984 | N | ALA | A | 143 | 2.522 | 47.557 | −1.994 | 1.00 | 208.05 | A | N |
| ATOM | 985 | CA | ALA | A | 143 | 3.324 | 47.527 | −3.212 | 1.00 | 206.37 | A | C |
| ATOM | 986 | CB | ALA | A | 143 | 3.588 | 48.942 | −3.703 | 1.00 | 216.49 | A | C |
| ATOM | 987 | C | ALA | A | 143 | 4.641 | 46.777 | −3.023 | 1.00 | 217.21 | A | C |
| ATOM | 988 | O | ALA | A | 143 | 4.967 | 45.877 | −3.800 | 1.00 | 198.58 | A | O |
| ATOM | 989 | N | ILE | A | 144 | 5.374 | 47.149 | −1.972 | 1.00 | 248.48 | A | N |
| ATOM | 990 | CA | ILE | A | 144 | 6.744 | 46.665 | −1.737 | 1.00 | 209.00 | A | C |
| ATOM | 991 | CB | ILE | A | 144 | 7.552 | 47.642 | −0.839 | 1.00 | 219.83 | A | C |
| ATOM | 992 | CG1 | ILE | A | 144 | 6.989 | 47.665 | 0.593 | 1.00 | 213.55 | A | C |
| ATOM | 993 | CD1 | ILE | A | 144 | 7.493 | 48.798 | 1.465 | 1.00 | 186.23 | A | C |
| ATOM | 994 | CG2 | ILE | A | 144 | 7.596 | 49.040 | −1.462 | 1.00 | 159.19 | A | C |
| ATOM | 995 | C | ILE | A | 144 | 6.757 | 45.257 | −1.171 | 1.00 | 178.50 | A | C |
| ATOM | 996 | O | ILE | A | 144 | 7.763 | 44.552 | −1.263 | 1.00 | 214.09 | A | O |
| ATOM | 997 | N | ILE | A | 145 | 5.634 | 44.852 | −0.593 | 1.00 | 195.26 | A | N |
| ATOM | 998 | CA | ILE | A | 145 | 5.444 | 43.475 | −0.163 | 1.00 | 181.20 | A | C |
| ATOM | 999 | CB | ILE | A | 145 | 4.112 | 43.311 | 0.582 | 1.00 | 166.45 | A | C |
| ATOM | 1000 | CG1 | ILE | A | 145 | 4.236 | 43.861 | 2.007 | 1.00 | 201.76 | A | C |
| ATOM | 1001 | CD1 | ILE | A | 145 | 2.899 | 44.233 | 2.620 | 1.00 | 219.00 | A | C |
| ATOM | 1002 | CG2 | ILE | A | 145 | 3.684 | 41.861 | 0.593 | 1.00 | 172.44 | A | C |
| ATOM | 1003 | C | ILE | A | 145 | 5.478 | 42.527 | −1.355 | 1.00 | 179.13 | A | C |
| ATOM | 1004 | O | ILE | A | 145 | 6.217 | 41.539 | −1.334 | 1.00 | 165.71 | A | O |
| ATOM | 1005 | N | TRP | A | 146 | 4.676 | 42.827 | −2.383 | 1.00 | 198.70 | A | N |
| ATOM | 1006 | CA | TRP | A | 146 | 4.589 | 41.996 | −3.594 | 1.00 | 188.01 | A | C |
| ATOM | 1007 | CB | TRP | A | 146 | 3.663 | 42.639 | −4.605 | 1.00 | 200.84 | A | C |
| ATOM | 1008 | CG | TRP | A | 146 | 2.215 | 42.322 | −4.363 | 1.00 | 223.12 | A | C |
| ATOM | 1009 | CD1 | TRP | A | 146 | 1.216 | 43.185 | −3.925 | 1.00 | 219.21 | A | C |
| ATOM | 1010 | NE1 | TRP | A | 146 | 0.010 | 42.531 | −3.826 | 1.00 | 234.83 | A | N |
| ATOM | 1011 | CE2 | TRP | A | 146 | 0.143 | 41.233 | −4.177 | 1.00 | 253.16 | A | C |
| ATOM | 1012 | CD2 | TRP | A | 146 | 1.552 | 41.022 | −4.532 | 1.00 | 251.03 | A | C |
| ATOM | 1013 | CE3 | TRP | A | 146 | 1.967 | 39.751 | −4.935 | 1.00 | 240.01 | A | C |
| ATOM | 1014 | CZ3 | TRP | A | 146 | 1.020 | 38.723 | −4.976 | 1.00 | 215.20 | A | C |
| ATOM | 1015 | CH2 | TRP | A | 146 | −0.322 | 38.942 | −4.640 | 1.00 | 194.13 | A | C |
| ATOM | 1016 | CZ2 | TRP | A | 146 | −0.787 | 40.202 | −4.228 | 1.00 | 226.47 | A | C |
| ATOM | 1017 | C | TRP | A | 146 | 5.921 | 41.755 | −4.244 | 1.00 | 172.50 | A | C |
| ATOM | 1018 | O | TRP | A | 146 | 6.194 | 40.666 | −4.754 | 1.00 | 154.52 | A | O |
| ATOM | 1019 | N | LEU | A | 147 | 6.751 | 42.789 | −4.243 | 1.00 | 213.49 | A | N |
| ATOM | 1020 | CA | LEU | A | 147 | 8.094 | 42.697 | −4.774 | 1.00 | 202.69 | A | C |
| ATOM | 1021 | CB | LEU | A | 147 | 8.647 | 44.087 | −5.022 | 1.00 | 202.67 | A | C |
| ATOM | 1022 | CG | LEU | A | 147 | 7.862 | 44.924 | −6.026 | 1.00 | 184.58 | A | C |
| ATOM | 1023 | CD1 | LEU | A | 147 | 8.469 | 46.314 | −6.073 | 1.00 | 217.03 | A | C |
| ATOM | 1024 | CD2 | LEU | A | 147 | 7.827 | 44.271 | −7.405 | 1.00 | 184.22 | A | C |
| ATOM | 1025 | C | LEU | A | 147 | 8.986 | 41.933 | −3.820 | 1.00 | 207.94 | A | C |
| ATOM | 1026 | O | LEU | A | 147 | 9.908 | 41.256 | −4.268 | 1.00 | 207.72 | A | O |
| ATOM | 1027 | N | ALA | A | 148 | 8.698 | 42.023 | −2.518 | 1.00 | 207.27 | A | N |
| ATOM | 1028 | CA | ALA | A | 148 | 9.431 | 41.262 | −1.489 | 1.00 | 201.43 | A | C |
| ATOM | 1029 | CB | ALA | A | 148 | 9.116 | 41.786 | −0.090 | 1.00 | 196.62 | A | C |
| ATOM | 1030 | C | ALA | A | 148 | 9.142 | 39.756 | −1.584 | 1.00 | 201.33 | A | C |
| ATOM | 1031 | O | ALA | A | 148 | 10.056 | 38.917 | −1.506 | 1.00 | 191.80 | A | O |
| ATOM | 1032 | N | TRP | A | 149 | 7.863 | 39.418 | −1.751 | 1.00 | 182.10 | A | N |
| ATOM | 1033 | CA | TRP | A | 149 | 7.435 | 38.036 | −1.947 | 1.00 | 167.07 | A | C |
| ATOM | 1034 | CB | TRP | A | 149 | 5.927 | 37.905 | −1.864 | 1.00 | 155.92 | A | C |
| ATOM | 1035 | CG | TRP | A | 149 | 5.394 | 38.044 | −0.463 | 1.00 | 171.82 | A | C |
| ATOM | 1036 | CD1 | TRP | A | 149 | 6.108 | 37.985 | 0.733 | 1.00 | 191.30 | A | C |
| ATOM | 1037 | NE1 | TRP | A | 149 | 5.278 | 38.134 | 1.815 | 1.00 | 195.77 | A | N |
| ATOM | 1038 | CE2 | TRP | A | 149 | 4.000 | 38.291 | 1.411 | 1.00 | 198.38 | A | C |
| ATOM | 1039 | CD2 | TRP | A | 149 | 4.001 | 38.238 | −0.063 | 1.00 | 193.06 | A | C |
| ATOM | 1040 | CE3 | TRP | A | 149 | 2.802 | 38.375 | −0.746 | 1.00 | 175.12 | A | C |
| ATOM | 1041 | CZ3 | TRP | A | 149 | 1.634 | 38.559 | 0.000 | 1.00 | 200.12 | A | C |
| ATOM | 1042 | CH2 | TRP | A | 149 | 1.649 | 38.608 | 1.413 | 1.00 | 219.67 | A | C |
| ATOM | 1043 | CZ2 | TRP | A | 149 | 2.834 | 38.475 | 2.143 | 1.00 | 176.46 | A | C |
| ATOM | 1044 | C | TRP | A | 149 | 7.914 | 37.529 | −3.248 | 1.00 | 166.45 | A | C |
| ATOM | 1045 | O | TRP | A | 149 | 8.404 | 36.419 | −3.312 | 1.00 | 194.49 | A | O |
| ATOM | 1046 | N | GLY | A | 150 | 7.783 | 38.331 | −4.299 | 1.00 | 180.00 | A | N |
| ATOM | 1047 | CA | GLY | A | 150 | 8.371 | 38.000 | −5.597 | 1.00 | 185.95 | A | C |
| ATOM | 1048 | C | GLY | A | 150 | 9.843 | 37.664 | −5.460 | 1.00 | 202.56 | A | C |
| ATOM | 1049 | O | GLY | A | 150 | 10.367 | 36.812 | −6.196 | 1.00 | 199.47 | A | O |
| ATOM | 1050 | N | VAL | A | 151 | 10.495 | 38.314 | −4.493 | 1.00 | 204.53 | A | N |
| ATOM | 1051 | CA | VAL | A | 151 | 11.925 | 38.126 | −4.260 | 1.00 | 202.90 | A | C |
| ATOM | 1052 | CB | VAL | A | 151 | 12.487 | 39.213 | −3.316 | 1.00 | 202.41 | A | C |
| ATOM | 1053 | CG1 | VAL | A | 151 | 13.576 | 38.653 | −2.418 | 1.00 | 212.19 | A | C |
| ATOM | 1054 | CG2 | VAL | A | 151 | 13.012 | 40.389 | −4.128 | 1.00 | 225.44 | A | C |
| ATOM | 1055 | C | VAL | A | 151 | 12.232 | 36.730 | −3.741 | 1.00 | 197.13 | A | C |
| ATOM | 1056 | O | VAL | A | 151 | 13.009 | 35.990 | −4.354 | 1.00 | 208.08 | A | O |
| ATOM | 1057 | N | LEU | A | 152 | 11.623 | 36.377 | −2.609 | 1.00 | 193.85 | A | N |
| ATOM | 1058 | CA | LEU | A | 152 | 11.732 | 35.020 | −2.087 | 1.00 | 190.02 | A | C |
| ATOM | 1059 | CB | LEU | A | 152 | 10.742 | 34.787 | −0.956 | 1.00 | 179.06 | A | C |
| ATOM | 1060 | CG | LEU | A | 152 | 11.330 | 34.629 | 0.445 | 1.00 | 157.47 | A | C |
| ATOM | 1061 | CD1 | LEU | A | 152 | 10.203 | 34.469 | 1.444 | 1.00 | 166.04 | A | C |
| ATOM | 1062 | CD2 | LEU | A | 152 | 12.305 | 33.470 | 0.525 | 1.00 | 152.45 | A | C |
| ATOM | 1063 | C | LEU | A | 152 | 11.539 | 33.989 | −3.183 | 1.00 | 176.89 | A | C |
| ATOM | 1064 | O | LEU | A | 152 | 12.432 | 33.199 | −3.450 | 1.00 | 193.92 | A | O |
| ATOM | 1065 | N | TRP | A | 153 | 10.383 | 34.026 | −3.837 | 1.00 | 181.15 | A | N |
| ATOM | 1066 | CA | TRP | A | 153 | 10.017 | 33.022 | −4.844 | 1.00 | 192.69 | A | C |
| ATOM | 1067 | CB | TRP | A | 153 | 8.649 | 33.349 | −5.442 | 1.00 | 196.33 | A | C |
| ATOM | 1068 | CG | TRP | A | 153 | 7.515 | 32.775 | −4.649 | 1.00 | 192.38 | A | C |
| ATOM | 1069 | CD1 | TRP | A | 153 | 6.548 | 31.881 | −5.089 | 1.00 | 182.76 | A | C |
| ATOM | 1070 | NE1 | TRP | A | 153 | 5.696 | 31.546 | −4.072 | 1.00 | 165.14 | A | N |
| ATOM | 1071 | CE2 | TRP | A | 153 | 6.052 | 32.147 | −2.922 | 1.00 | 155.40 | A | C |
| ATOM | 1072 | CD2 | TRP | A | 153 | 7.226 | 32.972 | −3.218 | 1.00 | 174.16 | A | C |
| ATOM | 1073 | CE3 | TRP | A | 153 | 7.811 | 33.704 | −2.191 | 1.00 | 179.63 | A | C |
| ATOM | 1074 | CZ3 | TRP | A | 153 | 7.235 | 33.647 | −0.916 | 1.00 | 182.29 | A | C |
| ATOM | 1075 | CH2 | TRP | A | 153 | 6.096 | 32.864 | −0.659 | 1.00 | 171.17 | A | C |
| ATOM | 1076 | CZ2 | TRP | A | 153 | 5.487 | 32.098 | −1.656 | 1.00 | 160.04 | A | C |
| ATOM | 1077 | C | TRP | A | 153 | 11.046 | 32.897 | −5.922 | 1.00 | 160.13 | A | C |
| ATOM | 1078 | O | TRP | A | 153 | 11.367 | 31.793 | −6.366 | 1.00 | 183.39 | A | O |
| ATOM | 1079 | N | LEU | A | 154 | 11.594 | 34.039 | −6.335 | 1.00 | 196.79 | A | N |
| ATOM | 1080 | CA | LEU | A | 154 | 12.596 | 34.088 | −7.380 | 1.00 | 205.40 | A | C |
| ATOM | 1081 | CB | LEU | A | 154 | 12.871 | 35.542 | −7.742 | 1.00 | 193.98 | A | C |
| ATOM | 1082 | CG | LEU | A | 154 | 13.593 | 35.765 | −9.057 | 1.00 | 242.51 | A | C |
| ATOM | 1083 | CD1 | LEU | A | 154 | 13.069 | 34.834 | −10.135 | 1.00 | 215.01 | A | C |
| ATOM | 1084 | CD2 | LEU | A | 154 | 13.387 | 37.211 | −9.474 | 1.00 | 294.65 | A | C |
| ATOM | 1085 | C | LEU | A | 154 | 13.876 | 33.337 | −6.984 | 1.00 | 209.63 | A | C |
| ATOM | 1086 | O | LEU | A | 154 | 14.530 | 32.720 | −7.827 | 1.00 | 199.48 | A | O |
| ATOM | 1087 | N | THR | A | 155 | 14.215 | 33.377 | −5.697 | 1.00 | 214.33 | A | N |
| ATOM | 1088 | CA | THR | A | 155 | 15.431 | 32.741 | −5.199 | 1.00 | 189.27 | A | C |
| ATOM | 1089 | CB | THR | A | 155 | 15.580 | 32.880 | −3.674 | 1.00 | 187.11 | A | C |
| ATOM | 1090 | OG1 | THR | A | 155 | 14.551 | 32.123 | −3.009 | 1.00 | 175.43 | A | O |
| ATOM | 1091 | CG2 | THR | A | 155 | 15.526 | 34.354 | −3.260 | 1.00 | 177.05 | A | C |
| ATOM | 1092 | C | THR | A | 155 | 15.435 | 31.270 | −5.566 | 1.00 | 191.89 | A | C |
| ATOM | 1093 | O | THR | A | 155 | 16.422 | 30.754 | −6.085 | 1.00 | 203.18 | A | O |
| ATOM | 1094 | N | ALA | A | 156 | 14.314 | 30.598 | −5.305 | 1.00 | 171.95 | A | N |
| ATOM | 1095 | CA | ALA | A | 156 | 14.131 | 29.239 | −5.754 | 1.00 | 160.36 | A | C |
| ATOM | 1096 | CB | ALA | A | 156 | 12.714 | 28.791 | −5.511 | 1.00 | 160.92 | A | C |
| ATOM | 1097 | C | ALA | A | 156 | 14.482 | 29.113 | −7.234 | 1.00 | 192.92 | A | C |
| ATOM | 1098 | O | ALA | A | 156 | 15.025 | 28.092 | −7.640 | 1.00 | 204.38 | A | O |
| ATOM | 1099 | N | PHE | A | 157 | 14.208 | 30.145 | −8.028 | 1.00 | 188.96 | A | N |
| ATOM | 1100 | CA | PHE | A | 157 | 14.446 | 30.060 | −9.463 | 1.00 | 194.56 | A | C |
| ATOM | 1101 | CB | PHE | A | 157 | 13.749 | 31.175 | −10.228 | 1.00 | 183.75 | A | C |
| ATOM | 1102 | CG | PHE | A | 157 | 14.266 | 31.343 | −11.610 | 1.00 | 194.29 | A | C |
| ATOM | 1103 | CD1 | PHE | A | 157 | 13.919 | 30.463 | −12.617 | 1.00 | 218.94 | A | C |
| ATOM | 1104 | CE1 | PHE | A | 157 | 14.417 | 30.611 | −13.900 | 1.00 | 209.39 | A | C |
| ATOM | 1105 | CZ | PHE | A | 157 | 15.288 | 31.661 | −14.195 | 1.00 | 242.38 | A | C |
| ATOM | 1106 | CE2 | PHE | A | 157 | 15.658 | 32.542 | −13.191 | 1.00 | 260.31 | A | C |
| ATOM | 1107 | CD2 | PHE | A | 157 | 15.147 | 32.379 | −11.907 | 1.00 | 242.49 | A | C |
| ATOM | 1108 | C | PHE | A | 157 | 15.915 | 30.041 | −9.827 | 1.00 | 196.20 | A | C |
| ATOM | 1109 | O | PHE | A | 157 | 16.391 | 29.069 | −10.384 | 1.00 | 217.71 | A | O |
| ATOM | 1110 | N | ILE | A | 158 | 16.620 | 31.120 | −9.521 | 1.00 | 236.91 | A | N |
| ATOM | 1111 | CA | ILE | A | 158 | 18.032 | 31.251 | −9.881 | 1.00 | 240.24 | A | C |
| ATOM | 1112 | CB | ILE | A | 158 | 18.675 | 32.537 | −9.307 | 1.00 | 234.22 | A | C |
| ATOM | 1113 | CG1 | ILE | A | 158 | 18.288 | 32.745 | −7.840 | 1.00 | 206.47 | A | C |
| ATOM | 1114 | CD1 | ILE | A | 158 | 19.277 | 33.535 | −7.008 | 1.00 | 196.99 | A | C |
| ATOM | 1115 | CG2 | ILE | A | 158 | 18.271 | 33.740 | −10.146 | 1.00 | 235.21 | A | C |
| ATOM | 1116 | C | ILE | A | 158 | 18.814 | 30.060 | −9.385 | 1.00 | 219.24 | A | C |
| ATOM | 1117 | O | ILE | A | 158 | 19.514 | 29.396 | −10.152 | 1.00 | 217.05 | A | O |
| ATOM | 1118 | N | GLU | A | 159 | 18.656 | 29.785 | −8.095 | 1.00 | 231.04 | A | N |
| ATOM | 1119 | CA | GLU | A | 159 | 19.427 | 28.763 | −7.418 | 1.00 | 238.35 | A | C |
| ATOM | 1120 | CB | GLU | A | 159 | 19.083 | 28.725 | −5.932 | 1.00 | 241.49 | A | C |
| ATOM | 1121 | CG | GLU | A | 159 | 19.962 | 27.781 | −5.133 | 1.00 | 201.81 | A | C |
| ATOM | 1122 | CD | GLU | A | 159 | 20.056 | 28.175 | −3.670 | 1.00 | 241.93 | A | C |
| ATOM | 1123 | OE1 | GLU | A | 159 | 19.026 | 28.518 | −3.040 | 1.00 | 263.36 | A | O |
| ATOM | 1124 | OE2 | GLU | A | 159 | 21.182 | 28.147 | −3.144 | 1.00 | 232.78 | A | O |
| ATOM | 1125 | C | GLU | A | 159 | 19.248 | 27.397 | −8.026 | 1.00 | 219.69 | A | C |
| ATOM | 1126 | O | GLU | A | 159 | 20.212 | 26.648 | −8.174 | 1.00 | 194.88 | A | O |
| ATOM | 1127 | N | ASN | A | 160 | 18.014 | 27.091 | −8.391 | 1.00 | 204.33 | A | N |
| ATOM | 1128 | CA | ASN | A | 160 | 17.663 | 25.740 | −8.767 | 1.00 | 188.07 | A | C |
| ATOM | 1129 | CB | ASN | A | 160 | 16.297 | 25.352 | −8.186 | 1.00 | 193.61 | A | C |
| ATOM | 1130 | CG | ASN | A | 160 | 16.383 | 24.879 | −6.744 | 1.00 | 181.59 | A | C |
| ATOM | 1131 | OD1 | ASN | A | 160 | 17.271 | 24.102 | −6.392 | 1.00 | 202.16 | A | O |
| ATOM | 1132 | ND2 | ASN | A | 160 | 15.445 | 25.331 | −5.904 | 1.00 | 155.53 | A | N |
| ATOM | 1133 | C | ASN | A | 160 | 17.694 | 25.457 | −10.252 | 1.00 | 200.23 | A | C |
| ATOM | 1134 | O | ASN | A | 160 | 18.135 | 24.385 | −10.644 | 1.00 | 211.56 | A | O |
| ATOM | 1135 | N | ILE | A | 161 | 17.231 | 26.391 | −11.081 | 1.00 | 222.79 | A | N |
| ATOM | 1136 | CA | ILE | A | 161 | 16.913 | 26.021 | −12.464 | 1.00 | 227.06 | A | C |
| ATOM | 1137 | CB | ILE | A | 161 | 15.635 | 26.678 | −13.018 | 1.00 | 232.73 | A | C |
| ATOM | 1138 | CG1 | ILE | A | 161 | 15.228 | 25.959 | −14.304 | 1.00 | 236.91 | A | C |
| ATOM | 1139 | CD1 | ILE | A | 161 | 15.088 | 24.439 | −14.195 | 1.00 | 218.30 | A | C |
| ATOM | 1140 | CG2 | ILE | A | 161 | 15.851 | 28.156 | −13.263 | 1.00 | 223.08 | A | C |
| ATOM | 1141 | C | ILE | A | 161 | 18.039 | 26.066 | −13.488 | 1.00 | 263.08 | A | C |
| ATOM | 1142 | O | ILE | A | 161 | 18.209 | 25.124 | −14.264 | 1.00 | 293.14 | A | O |
| ATOM | 1143 | N | LEU | A | 162 | 18.772 | 27.171 | −13.524 | 1.00 | 241.15 | A | N |
| ATOM | 1144 | CA | LEU | A | 162 | 19.971 | 27.209 | −14.329 | 1.00 | 318.69 | A | C |
| ATOM | 1145 | CB | LEU | A | 162 | 19.658 | 27.191 | −15.834 | 1.00 | 364.85 | A | C |
| ATOM | 1146 | CG | LEU | A | 162 | 20.612 | 26.328 | −16.677 | 1.00 | 353.87 | A | C |
| ATOM | 1147 | CD1 | LEU | A | 162 | 20.737 | 24.910 | −16.106 | 1.00 | 211.60 | A | C |
| ATOM | 1148 | CD2 | LEU | A | 162 | 20.183 | 26.283 | −18.142 | 1.00 | 335.25 | A | C |
| ATOM | 1149 | C | LEU | A | 162 | 20.788 | 28.421 | −13.949 | 1.00 | 307.59 | A | C |
| ATOM | 1150 | O | LEU | A | 162 | 21.226 | 29.197 | −14.812 | 1.00 | 386.35 | A | O |
| ATOM | 1151 | N | LYS | A | 163 | 20.958 | 28.598 | −12.640 | 1.00 | 305.15 | A | N |
| ATOM | 1152 | CA | LYS | A | 163 | 21.963 | 29.528 | −12.139 | 1.00 | 300.54 | A | C |
| ATOM | 1153 | CB | LYS | A | 163 | 21.377 | 30.859 | −11.685 | 1.00 | 273.73 | A | C |
| ATOM | 1154 | CG | LYS | A | 163 | 22.449 | 31.677 | −10.989 | 1.00 | 258.67 | A | C |
| ATOM | 1155 | CD | LYS | A | 163 | 21.935 | 32.982 | −10.455 | 1.00 | 263.29 | A | C |
| ATOM | 1156 | CE | LYS | A | 163 | 23.076 | 34.014 | −10.251 | 1.00 | 332.17 | A | C |
| ATOM | 1157 | NZ | LYS | A | 163 | 23.011 | 35.427 | −10.869 | 1.00 | 398.09 | A | N |
| ATOM | 1158 | C | LYS | A | 163 | 22.853 | 28.994 | −11.024 | 1.00 | 285.22 | A | C |
| ATOM | 1159 | O | LYS | A | 163 | 23.996 | 29.434 | −10.907 | 1.00 | 310.48 | A | O |
| ATOM | 1160 | N | ILE | A | 164 | 22.325 | 28.115 | −10.179 | 1.00 | 240.53 | A | N |
| ATOM | 1161 | CA | ILE | A | 164 | 23.094 | 27.514 | −9.065 | 1.00 | 234.92 | A | C |
| ATOM | 1162 | CB | ILE | A | 164 | 23.859 | 26.232 | −9.515 | 1.00 | 242.82 | A | C |
| ATOM | 1163 | CG1 | ILE | A | 164 | 24.818 | 26.530 | −10.681 | 1.00 | 269.97 | A | C |
| ATOM | 1164 | CD1 | ILE | A | 164 | 25.908 | 25.494 | −10.901 | 1.00 | 306.72 | A | C |
| ATOM | 1165 | CG2 | ILE | A | 164 | 22.875 | 25.121 | −9.882 | 1.00 | 283.85 | A | C |
| ATOM | 1166 | C | ILE | A | 164 | 23.990 | 28.477 | −8.227 | 1.00 | 225.36 | A | C |
| ATOM | 1167 | O | ILE | A | 164 | 25.210 | 28.328 | −8.181 | 1.00 | 236.26 | A | O |
| ATOM | 1168 | N | PRO | A | 165 | 23.380 | 29.487 | −7.577 | 1.00 | 223.88 | A | N |
| ATOM | 1169 | CA | PRO | A | 165 | 24.041 | 30.390 | −6.648 | 1.00 | 215.86 | A | C |
| ATOM | 1170 | CB | PRO | A | 165 | 23.295 | 31.696 | −6.896 | 1.00 | 249.17 | A | C |
| ATOM | 1171 | CG | PRO | A | 165 | 21.901 | 31.217 | −7.083 | 1.00 | 242.75 | A | C |
| ATOM | 1172 | CD | PRO | A | 165 | 22.071 | 30.027 | −7.973 | 1.00 | 240.13 | A | C |
| ATOM | 1173 | C | PRO | A | 165 | 23.854 | 29.971 | −5.178 | 1.00 | 221.36 | A | C |
| ATOM | 1174 | O | PRO | A | 165 | 23.373 | 28.896 | −4.902 | 1.00 | 211.75 | A | O |
| ATOM | 1175 | N | LEU | A | 166 | 24.136 | 30.887 | −4.266 | 1.00 | 262.00 | A | N |
| ATOM | 1176 | CA | LEU | A | 166 | 24.304 | 30.623 | −2.837 | 1.00 | 265.07 | A | C |
| ATOM | 1177 | CB | LEU | A | 166 | 25.474 | 31.485 | −2.321 | 1.00 | 277.42 | A | C |
| ATOM | 1178 | CG | LEU | A | 166 | 26.483 | 32.170 | −3.254 | 1.00 | 268.61 | A | C |
| ATOM | 1179 | CD1 | LEU | A | 166 | 26.848 | 33.558 | −2.709 | 1.00 | 305.87 | A | C |
| ATOM | 1180 | CD2 | LEU | A | 166 | 27.674 | 31.241 | −3.426 | 1.00 | 281.86 | A | C |
| ATOM | 1181 | C | LEU | A | 166 | 23.055 | 30.987 | −2.018 | 1.00 | 225.67 | A | C |
| ATOM | 1182 | O | LEU | A | 166 | 22.253 | 31.858 | −2.428 | 1.00 | 229.94 | A | O |
| ATOM | 1183 | N | GLY | A | 167 | 22.937 | 30.382 | −0.838 | 1.00 | 209.59 | A | N |
| ATOM | 1184 | CA | GLY | A | 167 | 21.678 | 30.448 | −0.156 | 1.00 | 239.37 | A | C |
| ATOM | 1185 | C | GLY | A | 167 | 21.496 | 30.708 | 1.323 | 1.00 | 231.15 | A | C |
| ATOM | 1186 | O | GLY | A | 167 | 20.355 | 30.801 | 1.759 | 1.00 | 261.43 | A | O |
| ATOM | 1187 | N | LYS | A | 168 | 22.555 | 30.769 | 2.112 | 1.00 | 219.34 | A | N |
| ATOM | 1188 | CA | LYS | A | 168 | 22.404 | 30.814 | 3.595 | 1.00 | 228.99 | A | C |
| ATOM | 1189 | CB | LYS | A | 168 | 23.782 | 30.871 | 4.284 | 1.00 | 249.46 | A | C |
| ATOM | 1190 | CG | LYS | A | 168 | 23.866 | 31.520 | 5.670 | 1.00 | 263.58 | A | C |
| ATOM | 1191 | CD | LYS | A | 168 | 25.329 | 31.847 | 6.021 | 1.00 | 286.14 | A | C |
| ATOM | 1192 | CE | LYS | A | 168 | 25.526 | 33.199 | 6.711 | 1.00 | 293.99 | A | C |
| ATOM | 1193 | NZ | LYS | A | 168 | 26.939 | 33.654 | 6.531 | 1.00 | 390.13 | A | N |
| ATOM | 1194 | C | LYS | A | 168 | 21.397 | 31.838 | 4.193 | 1.00 | 208.27 | A | C |
| ATOM | 1195 | O | LYS | A | 168 | 20.937 | 31.636 | 5.314 | 1.00 | 223.71 | A | O |
| ATOM | 1196 | N | PHE | A | 169 | 21.066 | 32.911 | 3.467 | 1.00 | 213.54 | A | N |
| ATOM | 1197 | CA | PHE | A | 169 | 20.067 | 33.917 | 3.927 | 1.00 | 248.91 | A | C |
| ATOM | 1198 | CB | PHE | A | 169 | 20.579 | 35.397 | 3.770 | 1.00 | 308.25 | A | C |
| ATOM | 1199 | CG | PHE | A | 169 | 21.059 | 36.043 | 5.081 | 1.00 | 249.54 | A | C |
| ATOM | 1200 | CD1 | PHE | A | 169 | 22.249 | 35.607 | 5.685 | 1.00 | 303.40 | A | C |
| ATOM | 1201 | CE1 | PHE | A | 169 | 22.687 | 36.134 | 6.896 | 1.00 | 374.93 | A | C |
| ATOM | 1202 | CZ | PHE | A | 169 | 21.932 | 37.099 | 7.530 | 1.00 | 409.21 | A | C |
| ATOM | 1203 | CE2 | PHE | A | 169 | 20.766 | 37.576 | 6.947 | 1.00 | 333.45 | A | C |
| ATOM | 1204 | CD2 | PHE | A | 169 | 20.331 | 37.058 | 5.723 | 1.00 | 250.30 | A | C |
| ATOM | 1205 | C | PHE | A | 169 | 18.683 | 33.714 | 3.273 | 1.00 | 199.68 | A | C |
| ATOM | 1206 | O | PHE | A | 169 | 17.694 | 34.303 | 3.714 | 1.00 | 199.98 | A | O |
| ATOM | 1207 | N | THR | A | 170 | 18.629 | 32.895 | 2.212 | 1.00 | 218.61 | A | N |
| ATOM | 1208 | CA | THR | A | 170 | 17.358 | 32.553 | 1.537 | 1.00 | 225.48 | A | C |
| ATOM | 1209 | CB | THR | A | 170 | 17.539 | 31.513 | 0.411 | 1.00 | 189.02 | A | C |
| ATOM | 1210 | OG1 | THR | A | 170 | 18.567 | 31.945 | −0.480 | 1.00 | 217.60 | A | O |
| ATOM | 1211 | CG2 | THR | A | 170 | 16.233 | 31.301 | −0.364 | 1.00 | 157.09 | A | C |
| ATOM | 1212 | C | THR | A | 170 | 16.315 | 32.045 | 2.519 | 1.00 | 185.05 | A | C |
| ATOM | 1213 | O | THR | A | 170 | 15.138 | 32.376 | 2.390 | 1.00 | 194.96 | A | O |
| ATOM | 1214 | N | PRO | A | 171 | 16.738 | 31.228 | 3.494 | 1.00 | 181.15 | A | N |
| ATOM | 1215 | CA | PRO | A | 171 | 15.887 | 30.905 | 4.635 | 1.00 | 173.36 | A | C |
| ATOM | 1216 | CB | PRO | A | 171 | 16.717 | 29.872 | 5.414 | 1.00 | 149.91 | A | C |
| ATOM | 1217 | CG | PRO | A | 171 | 18.103 | 29.996 | 4.880 | 1.00 | 176.96 | A | C |
| ATOM | 1218 | CD | PRO | A | 171 | 17.942 | 30.388 | 3.456 | 1.00 | 187.73 | A | C |
| ATOM | 1219 | C | PRO | A | 171 | 15.567 | 32.145 | 5.489 | 1.00 | 197.86 | A | C |
| ATOM | 1220 | O | PRO | A | 171 | 14.416 | 32.354 | 5.879 | 1.00 | 213.37 | A | O |
| ATOM | 1221 | N | TRP | A | 172 | 16.570 | 32.971 | 5.754 | 1.00 | 188.09 | A | N |
| ATOM | 1222 | CA | TRP | A | 172 | 16.386 | 34.152 | 6.609 | 1.00 | 199.76 | A | C |
| ATOM | 1223 | CB | TRP | A | 172 | 17.725 | 34.816 | 6.933 | 1.00 | 193.63 | A | C |
| ATOM | 1224 | CG | TRP | A | 172 | 18.519 | 33.939 | 7.862 | 1.00 | 222.97 | A | C |
| ATOM | 1225 | CD1 | TRP | A | 172 | 19.650 | 33.186 | 7.562 | 1.00 | 220.49 | A | C |
| ATOM | 1226 | NE1 | TRP | A | 172 | 20.071 | 32.485 | 8.664 | 1.00 | 235.99 | A | N |
| ATOM | 1227 | CE2 | TRP | A | 172 | 19.259 | 32.720 | 9.730 | 1.00 | 242.14 | A | C |
| ATOM | 1228 | CD2 | TRP | A | 172 | 18.225 | 33.654 | 9.281 | 1.00 | 235.77 | A | C |
| ATOM | 1229 | CE3 | TRP | A | 172 | 17.246 | 34.077 | 10.193 | 1.00 | 201.35 | A | C |
| ATOM | 1230 | CZ3 | TRP | A | 172 | 17.292 | 33.587 | 11.505 | 1.00 | 179.67 | A | C |
| ATOM | 1231 | CH2 | TRP | A | 172 | 18.295 | 32.689 | 11.914 | 1.00 | 220.19 | A | C |
| ATOM | 1232 | CZ2 | TRP | A | 172 | 19.300 | 32.244 | 11.038 | 1.00 | 247.43 | A | C |
| ATOM | 1233 | C | TRP | A | 172 | 15.387 | 35.088 | 6.002 | 1.00 | 203.95 | A | C |
| ATOM | 1234 | O | TRP | A | 172 | 14.459 | 35.551 | 6.678 | 1.00 | 184.54 | A | O |
| ATOM | 1235 | N | LEU | A | 173 | 15.545 | 35.338 | 4.706 | 1.00 | 201.15 | A | N |
| ATOM | 1236 | CA | LEU | A | 173 | 14.584 | 36.097 | 3.930 | 1.00 | 184.45 | A | C |
| ATOM | 1237 | CB | LEU | A | 173 | 14.870 | 35.920 | 2.442 | 1.00 | 184.91 | A | C |
| ATOM | 1238 | CG | LEU | A | 173 | 13.968 | 36.674 | 1.477 | 1.00 | 180.47 | A | C |
| ATOM | 1239 | CD1 | LEU | A | 173 | 13.882 | 38.144 | 1.844 | 1.00 | 178.94 | A | C |
| ATOM | 1240 | CD2 | LEU | A | 173 | 14.465 | 36.507 | 0.058 | 1.00 | 196.04 | A | C |
| ATOM | 1241 | C | LEU | A | 173 | 13.178 | 35.630 | 4.252 | 1.00 | 186.41 | A | C |
| ATOM | 1242 | O | LEU | A | 173 | 12.292 | 36.434 | 4.512 | 1.00 | 185.18 | A | O |
| ATOM | 1243 | N | ALA | A | 174 | 12.992 | 34.315 | 4.253 | 1.00 | 180.24 | A | N |
| ATOM | 1244 | CA | ALA | A | 174 | 11.705 | 33.725 | 4.525 | 1.00 | 157.86 | A | C |
| ATOM | 1245 | CB | ALA | A | 174 | 11.722 | 32.271 | 4.111 | 1.00 | 108.00 | A | C |
| ATOM | 1246 | C | ALA | A | 174 | 11.305 | 33.856 | 5.992 | 1.00 | 192.16 | A | C |
| ATOM | 1247 | O | ALA | A | 174 | 10.113 | 33.924 | 6.309 | 1.00 | 199.09 | A | O |
| ATOM | 1248 | N | ILE | A | 175 | 12.293 | 33.890 | 6.884 | 1.00 | 179.45 | A | N |
| ATOM | 1249 | CA | ILE | A | 175 | 12.008 | 33.956 | 8.319 | 1.00 | 165.91 | A | C |
| ATOM | 1250 | CB | ILE | A | 175 | 13.197 | 33.512 | 9.177 | 1.00 | 163.96 | A | C |
| ATOM | 1251 | CG1 | ILE | A | 175 | 13.591 | 32.096 | 8.798 | 1.00 | 168.62 | A | C |
| ATOM | 1252 | CD1 | ILE | A | 175 | 15.058 | 31.804 | 8.963 | 1.00 | 199.14 | A | C |
| ATOM | 1253 | CG2 | ILE | A | 175 | 12.833 | 33.571 | 10.655 | 1.00 | 168.36 | A | C |
| ATOM | 1254 | C | ILE | A | 175 | 11.568 | 35.348 | 8.719 | 1.00 | 168.62 | A | C |
| ATOM | 1255 | O | ILE | A | 175 | 10.474 | 35.509 | 9.245 | 1.00 | 195.95 | A | O |
| ATOM | 1256 | N | ILE | A | 176 | 12.418 | 36.341 | 8.474 | 1.00 | 171.69 | A | N |
| ATOM | 1257 | CA | ILE | A | 176 | 12.017 | 37.738 | 8.626 | 1.00 | 188.32 | A | C |
| ATOM | 1258 | CB | ILE | A | 176 | 13.095 | 38.712 | 8.091 | 1.00 | 228.41 | A | C |
| ATOM | 1259 | CG1 | ILE | A | 176 | 13.356 | 38.433 | 6.606 | 1.00 | 196.43 | A | C |
| ATOM | 1260 | CD1 | ILE | A | 176 | 14.475 | 39.227 | 5.986 | 1.00 | 167.35 | A | C |
| ATOM | 1261 | CG2 | ILE | A | 176 | 14.373 | 38.657 | 8.937 | 1.00 | 214.56 | A | C |
| ATOM | 1262 | C | ILE | A | 176 | 10.684 | 38.025 | 7.915 | 1.00 | 197.51 | A | C |
| ATOM | 1263 | O | ILE | A | 176 | 9.768 | 38.611 | 8.509 | 1.00 | 188.28 | A | O |
| ATOM | 1264 | N | GLU | A | 177 | 10.582 | 37.592 | 6.654 | 1.00 | 190.96 | A | N |
| ATOM | 1265 | CA | GLU | A | 177 | 9.382 | 37.806 | 5.848 | 1.00 | 177.33 | A | C |
| ATOM | 1266 | CB | GLU | A | 177 | 9.570 | 37.304 | 4.420 | 1.00 | 176.35 | A | C |
| ATOM | 1267 | CG | GLU | A | 177 | 9.787 | 38.421 | 3.411 | 1.00 | 178.42 | A | C |
| ATOM | 1268 | CD | GLU | A | 177 | 9.696 | 37.914 | 1.992 | 1.00 | 164.65 | A | C |
| ATOM | 1269 | OE1 | GLU | A | 177 | 10.668 | 38.072 | 1.241 | 1.00 | 163.65 | A | O |
| ATOM | 1270 | OE2 | GLU | A | 177 | 8.657 | 37.336 | 1.630 | 1.00 | 180.79 | A | O |
| ATOM | 1271 | C | GLU | A | 177 | 8.162 | 37.157 | 6.477 | 1.00 | 202.58 | A | C |
| ATOM | 1272 | O | GLU | A | 177 | 7.029 | 37.580 | 6.231 | 1.00 | 194.98 | A | O |
| ATOM | 1273 | N | GLY | A | 178 | 8.408 | 36.125 | 7.282 | 1.00 | 187.34 | A | N |
| ATOM | 1274 | CA | GLY | A | 178 | 7.375 | 35.508 | 8.114 | 1.00 | 189.15 | A | C |
| ATOM | 1275 | C | GLY | A | 178 | 6.883 | 36.460 | 9.193 | 1.00 | 199.33 | A | C |
| ATOM | 1276 | O | GLY | A | 178 | 5.713 | 36.855 | 9.195 | 1.00 | 184.12 | A | O |
| ATOM | 1277 | N | ILE | A | 179 | 7.779 | 36.846 | 10.099 | 1.00 | 204.41 | A | N |
| ATOM | 1278 | CA | ILE | A | 179 | 7.434 | 37.789 | 11.173 | 1.00 | 202.23 | A | C |
| ATOM | 1279 | CB | ILE | A | 179 | 8.633 | 38.120 | 12.088 | 1.00 | 216.01 | A | C |
| ATOM | 1280 | CG1 | ILE | A | 179 | 9.194 | 36.854 | 12.742 | 1.00 | 192.16 | A | C |
| ATOM | 1281 | CD1 | ILE | A | 179 | 10.492 | 37.062 | 13.499 | 1.00 | 233.65 | A | C |
| ATOM | 1282 | CG2 | ILE | A | 179 | 8.194 | 39.105 | 13.163 | 1.00 | 204.36 | A | C |
| ATOM | 1283 | C | ILE | A | 179 | 6.882 | 39.103 | 10.629 | 1.00 | 184.30 | A | C |
| ATOM | 1284 | O | ILE | A | 179 | 5.859 | 39.619 | 11.101 | 1.00 | 185.33 | A | O |
| ATOM | 1285 | N | LEU | A | 180 | 7.571 | 39.636 | 9.630 | 1.00 | 190.76 | A | N |
| ATOM | 1286 | CA | LEU | A | 180 | 7.218 | 40.933 | 9.094 | 1.00 | 194.49 | A | C |
| ATOM | 1287 | CB | LEU | A | 180 | 8.415 | 41.580 | 8.375 | 1.00 | 200.71 | A | C |
| ATOM | 1288 | CG | LEU | A | 180 | 9.288 | 42.700 | 8.939 | 1.00 | 190.54 | A | C |
| ATOM | 1289 | CD1 | LEU | A | 180 | 10.067 | 42.162 | 10.132 | 1.00 | 239.74 | A | C |
| ATOM | 1290 | CD2 | LEU | A | 180 | 10.229 | 43.205 | 7.832 | 1.00 | 164.21 | A | C |
| ATOM | 1291 | C | LEU | A | 180 | 6.047 | 40.891 | 8.130 | 1.00 | 190.86 | A | C |
| ATOM | 1292 | O | LEU | A | 180 | 4.981 | 41.437 | 8.416 | 1.00 | 183.09 | A | O |
| ATOM | 1293 | N | THR | A | 181 | 6.256 | 40.231 | 6.992 | 1.00 | 197.29 | A | N |
| ATOM | 1294 | CA | THR | A | 181 | 5.464 | 40.493 | 5.789 | 1.00 | 202.73 | A | C |
| ATOM | 1295 | CB | THR | A | 181 | 6.184 | 40.027 | 4.506 | 1.00 | 228.49 | A | C |
| ATOM | 1296 | OG1 | THR | A | 181 | 7.569 | 40.382 | 4.572 | 1.00 | 252.59 | A | O |
| ATOM | 1297 | CG2 | THR | A | 181 | 5.576 | 40.693 | 3.292 | 1.00 | 199.13 | A | C |
| ATOM | 1298 | C | THR | A | 181 | 4.090 | 39.876 | 5.846 | 1.00 | 176.52 | A | C |
| ATOM | 1299 | O | THR | A | 181 | 3.109 | 40.482 | 5.417 | 1.00 | 198.39 | A | O |
| ATOM | 1300 | N | ALA | A | 182 | 4.024 | 38.663 | 6.369 | 1.00 | 174.15 | A | N |
| ATOM | 1301 | CA | ALA | A | 182 | 2.761 | 37.992 | 6.480 | 1.00 | 175.63 | A | C |
| ATOM | 1302 | CB | ALA | A | 182 | 2.933 | 36.525 | 6.141 | 1.00 | 202.12 | A | C |
| ATOM | 1303 | C | ALA | A | 182 | 2.153 | 38.143 | 7.862 | 1.00 | 182.74 | A | C |
| ATOM | 1304 | O | ALA | A | 182 | 0.950 | 38.356 | 7.984 | 1.00 | 184.10 | A | O |
| ATOM | 1305 | N | TRP | A | 183 | 2.983 | 38.034 | 8.898 | 1.00 | 190.88 | A | N |
| ATOM | 1306 | CA | TRP | A | 183 | 2.499 | 37.798 | 10.257 | 1.00 | 173.65 | A | C |
| ATOM | 1307 | CB | TRP | A | 183 | 3.654 | 37.412 | 11.163 | 1.00 | 186.43 | A | C |
| ATOM | 1308 | CG | TRP | A | 183 | 3.249 | 36.995 | 12.560 | 1.00 | 175.85 | A | C |
| ATOM | 1309 | CD1 | TRP | A | 183 | 2.011 | 37.163 | 13.192 | 1.00 | 153.05 | A | C |
| ATOM | 1310 | NE1 | TRP | A | 183 | 2.040 | 36.657 | 14.462 | 1.00 | 155.91 | A | N |
| ATOM | 1311 | CE2 | TRP | A | 183 | 3.271 | 36.161 | 14.753 | 1.00 | 155.25 | A | C |
| ATOM | 1312 | CD2 | TRP | A | 183 | 4.104 | 36.347 | 13.563 | 1.00 | 183.13 | A | C |
| ATOM | 1313 | CE3 | TRP | A | 183 | 5.431 | 35.907 | 13.588 | 1.00 | 196.68 | A | C |
| ATOM | 1314 | CZ3 | TRP | A | 183 | 5.920 | 35.297 | 14.751 | 1.00 | 175.52 | A | C |
| ATOM | 1315 | CH2 | TRP | A | 183 | 5.105 | 35.125 | 15.880 | 1.00 | 186.31 | A | C |
| ATOM | 1316 | CZ2 | TRP | A | 183 | 3.767 | 35.559 | 15.904 | 1.00 | 139.35 | A | C |
| ATOM | 1317 | C | TRP | A | 183 | 1.782 | 38.965 | 10.847 | 1.00 | 170.69 | A | C |
| ATOM | 1318 | O | TRP | A | 183 | 0.613 | 38.876 | 11.193 | 1.00 | 185.79 | A | O |
| ATOM | 1319 | N | ILE | A | 184 | 2.500 | 40.069 | 11.000 | 1.00 | 177.37 | A | N |
| ATOM | 1320 | CA | ILE | A | 184 | 1.950 | 41.272 | 11.607 | 1.00 | 182.59 | A | C |
| ATOM | 1321 | CB | ILE | A | 184 | 3.070 | 42.284 | 11.893 | 1.00 | 170.94 | A | C |
| ATOM | 1322 | CG1 | ILE | A | 184 | 3.932 | 41.741 | 13.034 | 1.00 | 190.10 | A | C |
| ATOM | 1323 | CD1 | ILE | A | 184 | 5.372 | 42.159 | 12.947 | 1.00 | 192.55 | A | C |
| ATOM | 1324 | CG2 | ILE | A | 184 | 2.504 | 43.641 | 12.267 | 1.00 | 185.52 | A | C |
| ATOM | 1325 | C | ILE | A | 184 | 0.788 | 41.855 | 10.792 | 1.00 | 198.96 | A | C |
| ATOM | 1326 | O | ILE | A | 184 | −0.266 | 42.127 | 11.370 | 1.00 | 172.14 | A | O |
| ATOM | 1327 | N | PRO | A | 185 | 0.963 | 42.024 | 9.458 | 1.00 | 190.94 | A | N |
| ATOM | 1328 | CA | PRO | A | 185 | −0.087 | 42.546 | 8.564 | 1.00 | 180.52 | A | C |
| ATOM | 1329 | CB | PRO | A | 185 | 0.483 | 42.286 | 7.161 | 1.00 | 168.11 | A | C |
| ATOM | 1330 | CG | PRO | A | 185 | 1.943 | 42.449 | 7.376 | 1.00 | 159.83 | A | C |
| ATOM | 1331 | CD | PRO | A | 185 | 2.196 | 41.756 | 8.698 | 1.00 | 190.93 | A | C |
| ATOM | 1332 | C | PRO | A | 185 | −1.402 | 41.808 | 8.718 | 1.00 | 184.75 | A | C |
| ATOM | 1333 | O | PRO | A | 185 | −2.461 | 42.393 | 8.515 | 1.00 | 181.18 | A | O |
| ATOM | 1334 | N | ALA | A | 186 | −1.324 | 40.531 | 9.074 | 1.00 | 189.26 | A | N |
| ATOM | 1335 | CA | ALA | A | 186 | −2.510 | 39.744 | 9.349 | 1.00 | 187.75 | A | C |
| ATOM | 1336 | CB | ALA | A | 186 | −2.206 | 38.262 | 9.222 | 1.00 | 174.21 | A | C |
| ATOM | 1337 | C | ALA | A | 186 | −3.064 | 40.073 | 10.733 | 1.00 | 208.98 | A | C |
| ATOM | 1338 | O | ALA | A | 186 | −4.242 | 40.417 | 10.872 | 1.00 | 195.29 | A | O |
| ATOM | 1339 | N | TRP | A | 187 | −2.203 | 39.979 | 11.747 | 1.00 | 191.88 | A | N |
| ATOM | 1340 | CA | TRP | A | 187 | −2.575 | 40.296 | 13.110 | 1.00 | 186.77 | A | C |
| ATOM | 1341 | CB | TRP | A | 187 | −1.454 | 39.921 | 14.074 | 1.00 | 190.34 | A | C |
| ATOM | 1342 | CG | TRP | A | 187 | −1.845 | 40.176 | 15.494 | 1.00 | 200.15 | A | C |
| ATOM | 1343 | CD1 | TRP | A | 187 | −1.562 | 41.307 | 16.250 | 1.00 | 235.00 | A | C |
| ATOM | 1344 | NE1 | TRP | A | 187 | −2.110 | 41.213 | 17.501 | 1.00 | 254.87 | A | N |
| ATOM | 1345 | CE2 | TRP | A | 187 | −2.785 | 40.057 | 17.640 | 1.00 | 233.10 | A | C |
| ATOM | 1346 | CD2 | TRP | A | 187 | −2.660 | 39.330 | 16.373 | 1.00 | 198.92 | A | C |
| ATOM | 1347 | CE3 | TRP | A | 187 | −3.262 | 38.108 | 16.251 | 1.00 | 219.28 | A | C |
| ATOM | 1348 | CZ3 | TRP | A | 187 | −3.976 | 37.602 | 17.345 | 1.00 | 252.82 | A | C |
| ATOM | 1349 | CH2 | TRP | A | 187 | −4.079 | 38.303 | 18.549 | 1.00 | 202.58 | A | C |
| ATOM | 1350 | CZ2 | TRP | A | 187 | −3.493 | 39.545 | 18.717 | 1.00 | 189.06 | A | C |
| ATOM | 1351 | C | TRP | A | 187 | −2.960 | 41.748 | 13.188 | 1.00 | 187.43 | A | C |
| ATOM | 1352 | O | TRP | A | 187 | −3.709 | 42.159 | 14.075 | 1.00 | 184.49 | A | O |
| ATOM | 1353 | N | LEU | A | 188 | −2.480 | 42.520 | 12.216 | 1.00 | 188.34 | A | N |
| ATOM | 1354 | CA | LEU | A | 188 | −2.804 | 43.941 | 12.068 | 1.00 | 190.75 | A | C |
| ATOM | 1355 | CB | LEU | A | 188 | −1.545 | 44.705 | 11.663 | 1.00 | 182.74 | A | C |
| ATOM | 1356 | CG | LEU | A | 188 | −1.567 | 46.161 | 11.235 | 1.00 | 169.14 | A | C |
| ATOM | 1357 | CD1 | LEU | A | 188 | −0.230 | 46.747 | 11.604 | 1.00 | 161.72 | A | C |
| ATOM | 1358 | CD2 | LEU | A | 188 | −1.799 | 46.295 | 9.743 | 1.00 | 174.65 | A | C |
| ATOM | 1359 | C | LEU | A | 188 | −3.919 | 44.157 | 11.040 | 1.00 | 178.99 | A | C |
| ATOM | 1360 | O | LEU | A | 188 | −4.429 | 45.264 | 10.870 | 1.00 | 172.77 | A | O |
| ATOM | 1361 | N | LEU | A | 189 | −4.286 | 43.092 | 10.341 | 1.00 | 187.54 | A | N |
| ATOM | 1362 | CA | LEU | A | 189 | −5.447 | 43.132 | 9.469 | 1.00 | 189.94 | A | C |
| ATOM | 1363 | CB | LEU | A | 189 | −5.327 | 42.069 | 8.386 | 1.00 | 169.93 | A | C |
| ATOM | 1364 | CG | LEU | A | 189 | −5.771 | 42.489 | 6.993 | 1.00 | 186.19 | A | C |
| ATOM | 1365 | CD1 | LEU | A | 189 | −5.043 | 43.745 | 6.530 | 1.00 | 164.76 | A | C |
| ATOM | 1366 | CD2 | LEU | A | 189 | −5.518 | 41.342 | 6.031 | 1.00 | 184.15 | A | C |
| ATOM | 1367 | C | LEU | A | 189 | −6.716 | 42.908 | 10.285 | 1.00 | 191.28 | A | C |
| ATOM | 1368 | O | LEU | A | 189 | −7.815 | 43.251 | 9.853 | 1.00 | 188.01 | A | O |
| ATOM | 1369 | N | PHE | A | 190 | −6.556 | 42.317 | 11.468 | 1.00 | 181.47 | A | N |
| ATOM | 1370 | CA | PHE | A | 190 | −7.664 | 42.165 | 12.406 | 1.00 | 176.72 | A | C |
| ATOM | 1371 | CB | PHE | A | 190 | −7.318 | 41.184 | 13.540 | 1.00 | 168.60 | A | C |
| ATOM | 1372 | CG | PHE | A | 190 | −7.151 | 39.760 | 13.085 | 1.00 | 197.71 | A | C |
| ATOM | 1373 | CD1 | PHE | A | 190 | −8.113 | 39.158 | 12.281 | 1.00 | 209.25 | A | C |
| ATOM | 1374 | CE1 | PHE | A | 190 | −7.959 | 37.854 | 11.861 | 1.00 | 193.09 | A | C |
| ATOM | 1375 | CZ | PHE | A | 190 | −6.837 | 37.132 | 12.244 | 1.00 | 187.01 | A | C |
| ATOM | 1376 | CE2 | PHE | A | 190 | −5.875 | 37.714 | 13.048 | 1.00 | 165.54 | A | C |
| ATOM | 1377 | CD2 | PHE | A | 190 | −6.039 | 39.020 | 13.465 | 1.00 | 184.76 | A | C |
| ATOM | 1378 | C | PHE | A | 190 | −8.042 | 43.498 | 13.001 | 1.00 | 200.33 | A | C |
| ATOM | 1379 | O | PHE | A | 190 | −9.201 | 43.704 | 13.354 | 1.00 | 210.93 | A | O |
| ATOM | 1380 | N | ILE | A | 191 | −7.066 | 44.402 | 13.078 | 1.00 | 203.10 | A | N |
| ATOM | 1381 | CA | ILE | A | 191 | −7.169 | 45.617 | 13.884 | 1.00 | 190.75 | A | C |
| ATOM | 1382 | CB | ILE | A | 191 | −5.953 | 45.742 | 14.826 | 1.00 | 221.34 | A | C |
| ATOM | 1383 | CG1 | ILE | A | 191 | −6.050 | 44.676 | 15.934 | 1.00 | 249.86 | A | C |
| ATOM | 1384 | CD1 | ILE | A | 191 | −4.871 | 44.613 | 16.887 | 1.00 | 272.20 | A | C |
| ATOM | 1385 | CG2 | ILE | A | 191 | −5.854 | 47.147 | 15.410 | 1.00 | 222.39 | A | C |
| ATOM | 1386 | C | ILE | A | 191 | −7.376 | 46.894 | 13.074 | 1.00 | 207.21 | A | C |
| ATOM | 1387 | O | ILE | A | 191 | −8.333 | 47.624 | 13.323 | 1.00 | 235.39 | A | O |
| ATOM | 1388 | N | GLN | A | 192 | −6.471 | 47.181 | 12.142 | 1.00 | 204.84 | A | N |
| ATOM | 1389 | CA | GLN | A | 192 | −6.585 | 48.358 | 11.279 | 1.00 | 209.47 | A | C |
| ATOM | 1390 | CB | GLN | A | 192 | −5.226 | 49.023 | 11.060 | 1.00 | 198.36 | A | C |
| ATOM | 1391 | CG | GLN | A | 192 | −4.490 | 49.449 | 12.331 | 1.00 | 201.73 | A | C |
| ATOM | 1392 | CD | GLN | A | 192 | −5.048 | 50.703 | 13.003 | 1.00 | 216.29 | A | C |
| ATOM | 1393 | OE1 | GLN | A | 192 | −5.668 | 51.559 | 12.360 | 1.00 | 207.84 | A | O |
| ATOM | 1394 | NE2 | GLN | A | 192 | −4.798 | 50.834 | 14.308 | 1.00 | 261.94 | A | N |
| ATOM | 1395 | C | GLN | A | 192 | −7.196 | 47.930 | 9.952 | 1.00 | 194.52 | A | C |
| ATOM | 1396 | O | GLN | A | 192 | −7.602 | 48.760 | 9.144 | 1.00 | 205.53 | A | O |
| ATOM | 1397 | N | HIS | A | 193 | −7.252 | 46.621 | 9.728 | 1.00 | 225.76 | A | N |
| ATOM | 1398 | CA | HIS | A | 193 | −8.126 | 46.053 | 8.693 | 1.00 | 240.35 | A | C |
| ATOM | 1399 | CB | HIS | A | 193 | −9.516 | 46.697 | 8.743 | 1.00 | 251.29 | A | C |
| ATOM | 1400 | CG | HIS | A | 193 | −10.163 | 46.629 | 10.112 | 1.00 | 244.54 | A | C |
| ATOM | 1401 | ND1 | HIS | A | 193 | −11.506 | 46.627 | 10.298 | 1.00 | 246.11 | A | N |
| ATOM | 1402 | CE1 | HIS | A | 193 | −11.771 | 46.538 | 11.622 | 1.00 | 295.89 | A | C |
| ATOM | 1403 | NE2 | HIS | A | 193 | −10.603 | 46.498 | 12.277 | 1.00 | 227.95 | A | N |
| ATOM | 1404 | CD2 | HIS | A | 193 | −9.595 | 46.529 | 11.377 | 1.00 | 239.02 | A | C |
| ATOM | 1405 | C | HIS | A | 193 | −7.564 | 46.058 | 7.301 | 1.00 | 208.79 | A | C |
| ATOM | 1406 | O | HIS | A | 193 | −7.556 | 45.018 | 6.640 | 1.00 | 210.91 | A | O |
| ATOM | 1407 | N | TRP | A | 194 | −7.117 | 47.218 | 6.822 | 1.00 | 205.98 | A | N |
| ATOM | 1408 | CA | TRP | A | 194 | −6.343 | 47.236 | 5.580 | 1.00 | 197.14 | A | C |
| ATOM | 1409 | CB | TRP | A | 194 | −7.082 | 47.923 | 4.448 | 1.00 | 202.56 | A | C |
| ATOM | 1410 | CG | TRP | A | 194 | −8.030 | 46.991 | 3.736 | 1.00 | 206.74 | A | C |
| ATOM | 1411 | CD1 | TRP | A | 194 | −9.396 | 46.856 | 3.951 | 1.00 | 190.61 | A | C |
| ATOM | 1412 | NE1 | TRP | A | 194 | −9.933 | 45.902 | 3.122 | 1.00 | 182.62 | A | N |
| ATOM | 1413 | CE2 | TRP | A | 194 | −8.984 | 45.362 | 2.320 | 1.00 | 239.51 | A | C |
| ATOM | 1414 | CD2 | TRP | A | 194 | −7.713 | 46.014 | 2.670 | 1.00 | 230.70 | A | C |
| ATOM | 1415 | CE3 | TRP | A | 194 | −6.559 | 45.639 | 1.994 | 1.00 | 234.76 | A | C |
| ATOM | 1416 | CZ3 | TRP | A | 194 | −6.658 | 44.646 | 0.997 | 1.00 | 206.40 | A | C |
| ATOM | 1417 | CH2 | TRP | A | 194 | −7.883 | 44.028 | 0.679 | 1.00 | 197.51 | A | C |
| ATOM | 1418 | CZ2 | TRP | A | 194 | −9.068 | 44.373 | 1.334 | 1.00 | 235.29 | A | C |
| ATOM | 1419 | C | TRP | A | 194 | −4.993 | 47.830 | 5.775 | 1.00 | 210.27 | A | C |
| ATOM | 1420 | O | TRP | A | 194 | −4.815 | 48.698 | 6.627 | 1.00 | 214.81 | A | O |
| ATOM | 1421 | N | VAL | A | 195 | −4.019 | 47.339 | 5.005 | 1.00 | 231.96 | A | N |
| ATOM | 1422 | CA | VAL | A | 195 | −2.671 | 47.914 | 4.978 | 1.00 | 231.48 | A | C |
| ATOM | 1423 | CB | VAL | A | 195 | −2.732 | 49.402 | 4.532 | 1.00 | 213.25 | A | C |
| ATOM | 1424 | CG1 | VAL | A | 195 | −1.352 | 50.035 | 4.376 | 1.00 | 208.38 | A | C |
| ATOM | 1425 | CG2 | VAL | A | 195 | −3.523 | 49.527 | 3.250 | 1.00 | 221.63 | A | C |
| ATOM | 1426 | C | VAL | A | 195 | −2.017 | 47.812 | 6.365 | 1.00 | 243.32 | A | C |
| ATOM | 1427 | O | VAL | A | 195 | −2.628 | 47.398 | 7.358 | 1.00 | 215.25 | A | O |
| ATOM | 1428 | OXT | VAL | A | 195 | −0.846 | 48.147 | 6.534 | 1.00 | 259.06 | | O |

TABLE 5
Atomic coordinates for HpUreI trimer

| | | | | | | | | | | | | |
|---|---|---|---|---|---|---|---|---|---|---|---|---|
| ATOM | 1 | N | MET | A | 1 | −10.169 | 37.593 | 3.983 | 1.00 | 189.55 | A | N |
| ATOM | 2 | CA | MET | A | 1 | −9.673 | 36.376 | 4.694 | 1.00 | 182.75 | A | C |
| ATOM | 3 | CB | MET | A | 1 | −10.139 | 35.118 | 3.976 | 1.00 | 148.30 | A | C |
| ATOM | 4 | CG | MET | A | 1 | −11.613 | 34.831 | 4.200 | 1.00 | 188.90 | A | C |
| ATOM | 5 | SD | MET | A | 1 | −12.315 | 33.807 | 2.898 | 1.00 | 224.25 | A | S |
| ATOM | 6 | CE | MET | A | 1 | −11.586 | 32.231 | 3.332 | 1.00 | 167.40 | A | C |
| ATOM | 7 | C | MET | A | 1 | −8.161 | 36.350 | 4.857 | 1.00 | 192.03 | A | C |
| ATOM | 8 | O | MET | A | 1 | −7.597 | 35.370 | 5.361 | 1.00 | 173.37 | A | O |
| ATOM | 9 | N | LEU | A | 2 | −7.519 | 37.448 | 4.460 | 1.00 | 191.46 | A | N |
| ATOM | 10 | CA | LEU | A | 2 | −6.065 | 37.579 | 4.511 | 1.00 | 168.46 | A | C |
| ATOM | 11 | CB | LEU | A | 2 | −5.588 | 38.784 | 3.688 | 1.00 | 171.29 | A | C |
| ATOM | 12 | CG | LEU | A | 2 | −5.883 | 38.752 | 2.177 | 1.00 | 224.57 | A | C |
| ATOM | 13 | CD1 | LEU | A | 2 | −7.210 | 39.448 | 1.852 | 1.00 | 233.13 | A | C |
| ATOM | 14 | CD2 | LEU | A | 2 | −4.731 | 39.326 | 1.357 | 1.00 | 145.04 | A | C |
| ATOM | 15 | C | LEU | A | 2 | −5.552 | 37.652 | 5.943 | 1.00 | 177.19 | A | C |
| ATOM | 16 | O | LEU | A | 2 | −4.423 | 37.249 | 6.209 | 1.00 | 170.43 | A | O |
| ATOM | 17 | N | GLY | A | 3 | −6.388 | 38.147 | 6.857 | 1.00 | 179.69 | A | N |
| ATOM | 18 | CA | GLY | A | 3 | −6.110 | 38.078 | 8.294 | 1.00 | 180.10 | A | C |
| ATOM | 19 | C | GLY | A | 3 | −5.860 | 36.639 | 8.701 | 1.00 | 175.78 | A | C |
| ATOM | 20 | O | GLY | A | 3 | −4.866 | 36.331 | 9.367 | 1.00 | 191.20 | A | O |
| ATOM | 21 | N | LEU | A | 4 | −6.774 | 35.764 | 8.287 | 1.00 | 159.08 | A | N |
| ATOM | 22 | CA | LEU | A | 4 | −6.633 | 34.337 | 8.474 | 1.00 | 150.77 | A | C |
| ATOM | 23 | CB | LEU | A | 4 | −7.934 | 33.642 | 8.120 | 1.00 | 142.87 | A | C |
| ATOM | 24 | CG | LEU | A | 4 | −8.607 | 32.914 | 9.267 | 1.00 | 164.66 | A | C |
| ATOM | 25 | CD1 | LEU | A | 4 | −9.867 | 32.274 | 8.731 | 1.00 | 204.17 | A | C |
| ATOM | 26 | CD2 | LEU | A | 4 | −7.691 | 31.849 | 9.851 | 1.00 | 162.21 | A | C |
| ATOM | 27 | C | LEU | A | 4 | −5.505 | 33.761 | 7.628 | 1.00 | 157.72 | A | C |
| ATOM | 28 | O | LEU | A | 4 | −4.492 | 33.297 | 8.157 | 1.00 | 156.44 | A | O |
| ATOM | 29 | N | VAL | A | 5 | −5.687 | 33.804 | 6.312 | 1.00 | 152.14 | A | N |
| ATOM | 30 | CA | VAL | A | 5 | −4.753 | 33.185 | 5.388 | 1.00 | 146.78 | A | C |
| ATOM | 31 | CB | VAL | A | 5 | −5.062 | 33.554 | 3.919 | 1.00 | 155.76 | A | C |
| ATOM | 32 | CG1 | VAL | A | 5 | −3.958 | 33.044 | 2.997 | 1.00 | 132.99 | A | C |
| ATOM | 33 | CG2 | VAL | A | 5 | −6.408 | 32.996 | 3.495 | 1.00 | 163.24 | A | C |
| ATOM | 34 | C | VAL | A | 5 | −3.323 | 33.584 | 5.698 | 1.00 | 159.44 | A | C |
| ATOM | 35 | O | VAL | A | 5 | −2.456 | 32.719 | 5.837 | 1.00 | 161.23 | A | O |
| ATOM | 36 | N | LEU | A | 6 | −3.089 | 34.890 | 5.820 | 1.00 | 161.39 | A | N |
| ATOM | 37 | CA | LEU | A | 6 | −1.731 | 35.408 | 5.961 | 1.00 | 167.97 | A | C |
| ATOM | 38 | CB | LEU | A | 6 | −1.634 | 36.873 | 5.521 | 1.00 | 164.96 | A | C |
| ATOM | 39 | CG | LEU | A | 6 | −1.697 | 37.118 | 4.011 | 1.00 | 147.62 | A | C |
| ATOM | 40 | CD1 | LEU | A | 6 | −1.440 | 38.565 | 3.699 | 1.00 | 140.44 | A | C |
| ATOM | 41 | CD2 | LEU | A | 6 | −0.670 | 36.278 | 3.273 | 1.00 | 177.85 | A | C |
| ATOM | 42 | C | LEU | A | 6 | −1.149 | 35.199 | 7.351 | 1.00 | 155.24 | A | C |
| ATOM | 43 | O | LEU | A | 6 | 0.070 | 35.136 | 7.510 | 1.00 | 150.59 | A | O |
| ATOM | 44 | N | LEU | A | 7 | −2.012 | 35.075 | 8.352 | 1.00 | 141.49 | A | N |
| ATOM | 45 | CA | LEU | A | 7 | −1.545 | 34.659 | 9.665 | 1.00 | 126.16 | A | C |
| ATOM | 46 | CB | LEU | A | 7 | −2.713 | 34.473 | 10.627 | 1.00 | 158.65 | A | C |
| ATOM | 47 | CG | LEU | A | 7 | −2.334 | 33.808 | 11.958 | 1.00 | 168.51 | A | C |
| ATOM | 48 | CD1 | LEU | A | 7 | −1.305 | 34.643 | 12.723 | 1.00 | 177.07 | A | C |
| ATOM | 49 | CD2 | LEU | A | 7 | −3.564 | 33.539 | 12.816 | 1.00 | 180.29 | A | C |
| ATOM | 50 | C | LEU | A | 7 | −0.742 | 33.356 | 9.578 | 1.00 | 140.00 | A | C |
| ATOM | 51 | O | LEU | A | 7 | 0.318 | 33.217 | 10.192 | 1.00 | 159.40 | A | O |
| ATOM | 52 | N | TYR | A | 8 | −1.245 | 32.407 | 8.796 | 1.00 | 130.91 | A | N |
| ATOM | 53 | CA | TYR | A | 8 | −0.626 | 31.087 | 8.684 | 1.00 | 129.70 | A | C |
| ATOM | 54 | CB | TYR | A | 8 | −1.695 | 30.027 | 8.435 | 1.00 | 145.26 | A | C |
| ATOM | 55 | CG | TYR | A | 8 | −2.581 | 29.820 | 9.638 | 1.00 | 147.56 | A | C |
| ATOM | 56 | CD1 | TYR | A | 8 | −2.213 | 28.929 | 10.652 | 1.00 | 134.69 | A | C |
| ATOM | 57 | CE1 | TYR | A | 8 | −3.007 | 28.744 | 11.772 | 1.00 | 146.15 | A | C |
| ATOM | 58 | CZ | TYR | A | 8 | −4.181 | 29.471 | 11.889 | 1.00 | 183.63 | A | C |
| ATOM | 59 | OH | TYR | A | 8 | −4.985 | 29.298 | 12.984 | 1.00 | 191.15 | A | O |
| ATOM | 60 | CE2 | TYR | A | 8 | −4.562 | 30.373 | 10.906 | 1.00 | 162.20 | A | C |
| ATOM | 61 | CD2 | TYR | A | 8 | −3.763 | 30.545 | 9.791 | 1.00 | 145.08 | A | C |
| ATOM | 62 | C | TYR | A | 8 | 0.484 | 31.032 | 7.641 | 1.00 | 146.38 | A | C |
| ATOM | 63 | O | TYR | A | 8 | 1.463 | 30.301 | 7.807 | 1.00 | 148.45 | A | O |
| ATOM | 64 | N | VAL | A | 9 | 0.338 | 31.832 | 6.586 | 1.00 | 137.16 | A | N |
| ATOM | 65 | CA | VAL | A | 9 | 1.437 | 32.083 | 5.656 | 1.00 | 138.42 | A | C |
| ATOM | 66 | CB | VAL | A | 9 | 1.066 | 33.118 | 4.570 | 1.00 | 170.54 | A | C |
| ATOM | 67 | CG1 | VAL | A | 9 | 2.298 | 33.587 | 3.809 | 1.00 | 170.41 | A | C |
| ATOM | 68 | CG2 | VAL | A | 9 | 0.072 | 32.533 | 3.589 | 1.00 | 181.87 | A | C |
| ATOM | 69 | C | VAL | A | 9 | 2.648 | 32.567 | 6.442 | 1.00 | 150.13 | A | C |
| ATOM | 70 | O | VAL | A | 9 | 3.786 | 32.355 | 6.035 | 1.00 | 150.07 | A | O |
| ATOM | 71 | N | GLY | A | 10 | 2.386 | 33.207 | 7.579 | 1.00 | 140.61 | A | N |
| ATOM | 72 | CA | GLY | A | 10 | 3.442 | 33.591 | 8.500 | 1.00 | 156.72 | A | C |
| ATOM | 73 | C | GLY | A | 10 | 4.301 | 32.421 | 8.942 | 1.00 | 150.49 | A | C |
| ATOM | 74 | O | GLY | A | 10 | 5.503 | 32.367 | 8.647 | 1.00 | 156.48 | A | O |
| ATOM | 75 | N | ILE | A | 11 | 3.683 | 31.485 | 9.652 | 1.00 | 146.22 | A | N |
| ATOM | 76 | CA | ILE | A | 11 | 4.413 | 30.352 | 10.219 | 1.00 | 149.41 | A | C |
| ATOM | 77 | CB | ILE | A | 11 | 3.527 | 29.471 | 11.108 | 1.00 | 148.98 | A | C |
| ATOM | 78 | CG1 | ILE | A | 11 | 2.429 | 30.322 | 11.720 | 1.00 | 166.54 | A | C |
| ATOM | 79 | CD1 | ILE | A | 11 | 1.119 | 29.610 | 11.913 | 1.00 | 185.28 | A | C |
| ATOM | 80 | CG2 | ILE | A | 11 | 4.359 | 28.802 | 12.196 | 1.00 | 118.06 | A | C |
| ATOM | 81 | C | ILE | A | 11 | 5.003 | 29.493 | 9.125 | 1.00 | 140.67 | A | C |
| ATOM | 82 | O | ILE | A | 11 | 6.183 | 29.173 | 9.175 | 1.00 | 139.38 | A | O |
| ATOM | 83 | N | VAL | A | 12 | 4.188 | 29.136 | 8.135 | 1.00 | 142.85 | A | N |
| ATOM | 84 | CA | VAL | A | 12 | 4.635 | 28.262 | 7.057 | 1.00 | 135.78 | A | C |
| ATOM | 85 | CB | VAL | A | 12 | 3.529 | 27.994 | 6.031 | 1.00 | 129.76 | A | C |
| ATOM | 86 | CG1 | VAL | A | 12 | 3.207 | 29.244 | 5.239 | 1.00 | 132.88 | A | C |
| ATOM | 87 | CG2 | VAL | A | 12 | 3.949 | 26.878 | 5.087 | 1.00 | 155.07 | A | C |
| ATOM | 88 | C | VAL | A | 12 | 5.857 | 28.822 | 6.344 | 1.00 | 146.03 | A | C |
| ATOM | 89 | O | VAL | A | 12 | 6.734 | 28.058 | 5.926 | 1.00 | 154.87 | A | O |
| ATOM | 90 | N | LEU | A | 13 | 5.905 | 30.147 | 6.201 | 1.00 | 148.03 | A | N |
| ATOM | 91 | CA | LEU | A | 13 | 7.070 | 30.801 | 5.622 | 1.00 | 148.35 | A | C |
| ATOM | 92 | CB | LEU | A | 13 | 6.770 | 32.232 | 5.187 | 1.00 | 156.85 | A | C |
| ATOM | 93 | CG | LEU | A | 13 | 6.565 | 32.379 | 3.679 | 1.00 | 154.07 | A | C |
| ATOM | 94 | CD1 | LEU | A | 13 | 6.221 | 33.815 | 3.340 | 1.00 | 143.78 | A | C |
| ATOM | 95 | CD2 | LEU | A | 13 | 7.788 | 31.901 | 2.893 | 1.00 | 165.11 | A | C |
| ATOM | 96 | C | LEU | A | 13 | 8.251 | 30.776 | 6.566 | 1.00 | 148.81 | A | C |
| ATOM | 97 | O | LEU | A | 13 | 9.372 | 30.507 | 6.146 | 1.00 | 169.97 | A | O |
| ATOM | 98 | N | ILE | A | 14 | 7.997 | 31.054 | 7.840 | 1.00 | 155.02 | A | N |
| ATOM | 99 | CA | ILE | A | 14 | 9.009 | 30.882 | 8.880 | 1.00 | 167.87 | A | C |
| ATOM | 100 | CB | ILE | A | 14 | 8.489 | 31.335 | 10.262 | 1.00 | 157.37 | A | C |
| ATOM | 101 | CG1 | ILE | A | 14 | 8.622 | 32.842 | 10.406 | 1.00 | 174.79 | A | C |
| ATOM | 102 | CD1 | ILE | A | 14 | 8.076 | 33.382 | 11.708 | 1.00 | 182.61 | A | C |
| ATOM | 103 | CG2 | ILE | A | 14 | 9.259 | 30.665 | 11.386 | 1.00 | 141.08 | A | C |
| ATOM | 104 | C | ILE | A | 14 | 9.467 | 29.424 | 8.930 | 1.00 | 163.34 | A | C |
| ATOM | 105 | O | ILE | A | 14 | 10.665 | 29.153 | 8.996 | 1.00 | 180.25 | A | O |
| ATOM | 106 | N | SER | A | 15 | 8.510 | 28.495 | 8.881 | 1.00 | 151.68 | A | N |
| ATOM | 107 | CA | SER | A | 15 | 8.810 | 27.066 | 8.965 | 1.00 | 150.36 | A | C |
| ATOM | 108 | CB | SER | A | 15 | 7.537 | 26.231 | 8.807 | 1.00 | 157.43 | A | C |
| ATOM | 109 | OG | SER | A | 15 | 7.833 | 24.870 | 8.523 | 1.00 | 191.98 | A | O |
| ATOM | 110 | C | SER | A | 15 | 9.844 | 26.666 | 7.924 | 1.00 | 164.19 | A | C |
| ATOM | 111 | O | SER | A | 15 | 10.886 | 26.107 | 8.264 | 1.00 | 178.70 | A | O |
| ATOM | 112 | N | ASN | A | 16 | 9.558 | 26.968 | 6.660 | 1.00 | 160.47 | A | N |
| ATOM | 113 | CA | ASN | A | 16 | 10.464 | 26.642 | 5.561 | 1.00 | 170.93 | A | C |
| ATOM | 114 | CB | ASN | A | 16 | 9.848 | 27.088 | 4.240 | 1.00 | 140.88 | A | C |
| ATOM | 115 | CG | ASN | A | 16 | 8.788 | 26.122 | 3.741 | 1.00 | 160.78 | A | C |
| ATOM | 116 | OD1 | ASN | A | 16 | 8.896 | 24.891 | 3.891 | 1.00 | 194.12 | A | O |
| ATOM | 117 | ND2 | ASN | A | 16 | 7.744 | 26.682 | 3.137 | 1.00 | 166.48 | A | N |
| ATOM | 118 | C | ASN | A | 16 | 11.867 | 27.222 | 5.717 | 1.00 | 177.88 | A | C |
| ATOM | 119 | O | ASN | A | 16 | 12.864 | 26.573 | 5.361 | 1.00 | 175.51 | A | O |
| ATOM | 120 | N | GLY | A | 17 | 11.936 | 28.441 | 6.257 | 1.00 | 179.94 | A | N |
| ATOM | 121 | CA | GLY | A | 17 | 13.209 | 29.066 | 6.603 | 1.00 | 197.24 | A | C |
| ATOM | 122 | C | GLY | A | 17 | 14.022 | 28.247 | 7.597 | 1.00 | 197.96 | A | C |
| ATOM | 123 | O | GLY | A | 17 | 15.157 | 27.834 | 7.305 | 1.00 | 208.34 | A | O |
| ATOM | 124 | N | ILE | A | 18 | 13.439 | 28.004 | 8.770 | 1.00 | 147.11 | A | N |
| ATOM | 125 | CA | ILE | A | 18 | 14.101 | 27.224 | 9.808 | 1.00 | 163.48 | A | C |
| ATOM | 126 | CB | ILE | A | 18 | 13.215 | 27.095 | 11.054 | 1.00 | 176.57 | A | C |
| ATOM | 127 | CG1 | ILE | A | 18 | 12.949 | 28.490 | 11.634 | 1.00 | 165.53 | A | C |
| ATOM | 128 | CD1 | ILE | A | 18 | 11.817 | 28.526 | 12.639 | 1.00 | 189.92 | A | C |
| ATOM | 129 | CG2 | ILE | A | 18 | 13.875 | 26.211 | 12.102 | 1.00 | 174.10 | A | C |
| ATOM | 130 | C | ILE | A | 18 | 14.539 | 25.849 | 9.312 | 1.00 | 178.28 | A | C |
| ATOM | 131 | O | ILE | A | 18 | 15.631 | 25.393 | 9.641 | 1.00 | 177.82 | A | O |
| ATOM | 132 | N | CYS | A | 19 | 13.697 | 25.199 | 8.516 | 1.00 | 173.93 | A | N |
| ATOM | 133 | CA | CYS | A | 19 | 13.959 | 23.838 | 8.058 | 1.00 | 159.55 | A | C |
| ATOM | 134 | CB | CYS | A | 19 | 12.693 | 23.203 | 7.501 | 1.00 | 169.39 | A | C |
| ATOM | 135 | SG | CYS | A | 19 | 11.452 | 22.880 | 8.776 | 1.00 | 223.58 | A | S |
| ATOM | 136 | C | CYS | A | 19 | 15.079 | 23.748 | 7.037 | 1.00 | 154.66 | A | C |
| ATOM | 137 | O | CYS | A | 19 | 15.576 | 22.662 | 6.780 | 1.00 | 181.87 | A | O |
| ATOM | 138 | N | GLY | A | 20 | 15.459 | 24.876 | 6.443 | 1.00 | 151.64 | A | N |
| ATOM | 139 | CA | GLY | A | 20 | 16.650 | 24.932 | 5.593 | 1.00 | 181.42 | A | C |
| ATOM | 140 | C | GLY | A | 20 | 17.882 | 25.174 | 6.450 | 1.00 | 191.01 | A | C |
| ATOM | 141 | O | GLY | A | 20 | 19.027 | 24.959 | 6.001 | 1.00 | 223.22 | A | O |
| ATOM | 142 | N | LEU | A | 21 | 17.636 | 25.596 | 7.695 | 1.00 | 194.97 | A | N |
| ATOM | 143 | CA | LEU | A | 21 | 18.694 | 25.934 | 8.642 | 1.00 | 184.13 | A | C |
| ATOM | 144 | CB | LEU | A | 21 | 18.325 | 27.159 | 9.476 | 1.00 | 185.32 | A | C |
| ATOM | 145 | CG | LEU | A | 21 | 18.004 | 28.444 | 8.731 | 1.00 | 188.59 | A | C |
| ATOM | 146 | CD1 | LEU | A | 21 | 17.495 | 29.471 | 9.725 | 1.00 | 189.99 | A | C |
| ATOM | 147 | CD2 | LEU | A | 21 | 19.235 | 28.959 | 8.013 | 1.00 | 176.52 | A | C |
| ATOM | 148 | C | LEU | A | 21 | 18.998 | 24.767 | 9.549 | 1.00 | 162.91 | A | C |
| ATOM | 149 | O | LEU | A | 21 | 20.076 | 24.201 | 9.437 | 1.00 | 178.76 | A | O |
| ATOM | 150 | N | THR | A | 22 | 18.059 | 24.398 | 10.426 | 1.00 | 162.64 | A | N |
| ATOM | 151 | CA | THR | A | 22 | 18.232 | 23.201 | 11.269 | 1.00 | 186.37 | A | C |
| ATOM | 152 | CB | THR | A | 22 | 17.187 | 23.092 | 12.423 | 1.00 | 173.77 | A | C |
| ATOM | 153 | OG1 | THR | A | 22 | 15.875 | 23.453 | 11.961 | 1.00 | 164.05 | A | O |
| ATOM | 154 | CG2 | THR | A | 22 | 17.566 | 23.991 | 13.586 | 1.00 | 219.25 | A | C |
| ATOM | 155 | C | THR | A | 22 | 18.285 | 21.908 | 10.425 | 1.00 | 172.27 | A | C |
| ATOM | 156 | O | THR | A | 22 | 18.829 | 20.880 | 10.867 | 1.00 | 154.09 | A | O |
| ATOM | 157 | N | LYS | A | 23 | 17.735 | 21.996 | 9.208 | 1.00 | 171.28 | A | N |
| ATOM | 158 | CA | LYS | A | 23 | 17.718 | 20.932 | 8.172 | 1.00 | 159.90 | A | C |
| ATOM | 159 | CB | LYS | A | 23 | 19.040 | 20.809 | 7.434 | 1.00 | 183.46 | A | C |
| ATOM | 160 | CG | LYS | A | 23 | 19.077 | 21.803 | 6.278 | 1.00 | 164.70 | A | C |
| ATOM | 161 | CD | LYS | A | 23 | 20.350 | 21.672 | 5.476 | 1.00 | 171.55 | A | C |
| ATOM | 162 | CE | LYS | A | 23 | 21.414 | 22.665 | 5.960 | 1.00 | 205.55 | A | C |
| ATOM | 163 | NZ | LYS | A | 23 | 22.401 | 22.877 | 4.871 | 1.00 | 254.21 | A | N |
| ATOM | 164 | C | LYS | A | 23 | 17.095 | 19.598 | 8.505 | 1.00 | 167.44 | A | C |
| ATOM | 165 | O | LYS | A | 23 | 17.771 | 18.582 | 8.764 | 1.00 | 164.27 | A | O |
| ATOM | 166 | N | VAL | A | 24 | 15.775 | 19.616 | 8.426 | 1.00 | 147.89 | A | N |
| ATOM | 167 | CA | VAL | A | 24 | 14.999 | 18.524 | 8.935 | 1.00 | 162.46 | A | C |
| ATOM | 168 | CB | VAL | A | 24 | 13.980 | 19.019 | 9.998 | 1.00 | 146.65 | A | C |
| ATOM | 169 | CG1 | VAL | A | 24 | 13.134 | 20.129 | 9.468 | 1.00 | 126.47 | A | C |
| ATOM | 170 | CG2 | VAL | A | 24 | 13.099 | 17.885 | 10.498 | 1.00 | 186.26 | A | C |
| ATOM | 171 | C | VAL | A | 24 | 14.358 | 17.707 | 7.814 | 1.00 | 157.17 | A | C |
| ATOM | 172 | O | VAL | A | 24 | 13.945 | 18.248 | 6.775 | 1.00 | 170.62 | A | O |
| ATOM | 173 | N | ASP | A | 25 | 14.426 | 16.391 | 8.016 | 1.00 | 131.26 | A | N |
| ATOM | 174 | CA | ASP | A | 25 | 13.472 | 15.415 | 7.538 | 1.00 | 137.79 | A | C |
| ATOM | 175 | CB | ASP | A | 25 | 12.887 | 14.769 | 8.797 | 1.00 | 149.37 | A | C |
| ATOM | 176 | CG | ASP | A | 25 | 11.819 | 13.745 | 8.507 | 1.00 | 156.03 | A | C |
| ATOM | 177 | OD1 | ASP | A | 25 | 12.157 | 12.634 | 8.026 | 1.00 | 167.03 | A | O |
| ATOM | 178 | OD2 | ASP | A | 25 | 10.642 | 14.048 | 8.803 | 1.00 | 147.77 | A | O |
| ATOM | 179 | C | ASP | A | 25 | 12.363 | 16.006 | 6.640 | 1.00 | 150.16 | A | C |
| ATOM | 180 | O | ASP | A | 25 | 11.339 | 16.468 | 7.142 | 1.00 | 148.84 | A | O |
| ATOM | 181 | N | PRO | A | 26 | 12.555 | 15.964 | 5.306 | 1.00 | 114.11 | A | N |
| ATOM | 182 | CA | PRO | A | 26 | 11.660 | 16.628 | 4.370 | 1.00 | 133.86 | A | C |
| ATOM | 183 | CB | PRO | A | 26 | 12.134 | 16.121 | 3.006 | 1.00 | 130.45 | A | C |
| ATOM | 184 | CG | PRO | A | 26 | 13.545 | 15.732 | 3.230 | 1.00 | 124.90 | A | C |
| ATOM | 185 | CD | PRO | A | 26 | 13.538 | 15.130 | 4.601 | 1.00 | 120.32 | A | C |
| ATOM | 186 | C | PRO | A | 26 | 10.199 | 16.250 | 4.578 | 1.00 | 131.84 | A | C |
| ATOM | 187 | O | PRO | A | 26 | 9.312 | 17.049 | 4.251 | 1.00 | 143.98 | A | O |
| ATOM | 188 | N | LYS | A | 27 | 9.947 | 15.055 | 5.112 | 1.00 | 109.12 | A | N |
| ATOM | 189 | CA | LYS | A | 27 | 8.587 | 14.661 | 5.433 | 1.00 | 109.86 | A | C |
| ATOM | 190 | CB | LYS | A | 27 | 8.532 | 13.232 | 5.970 | 1.00 | 97.08 | A | C |
| ATOM | 191 | CG | LYS | A | 27 | 9.185 | 12.147 | 5.129 | 1.00 | 115.05 | A | C |
| ATOM | 192 | CD | LYS | A | 27 | 8.180 | 11.512 | 4.175 | 1.00 | 140.34 | A | C |
| ATOM | 193 | CE | LYS | A | 27 | 8.642 | 10.158 | 3.634 | 1.00 | 196.55 | A | C |
| ATOM | 194 | NZ | LYS | A | 27 | 7.691 | 9.516 | 2.662 | 1.00 | 178.81 | A | N |
| ATOM | 195 | C | LYS | A | 27 | 7.979 | 15.606 | 6.474 | 1.00 | 122.18 | A | C |
| ATOM | 196 | O | LYS | A | 27 | 6.810 | 15.959 | 6.388 | 1.00 | 139.18 | A | O |
| ATOM | 197 | N | SER | A | 28 | 8.768 | 16.011 | 7.465 | 1.00 | 130.86 | A | N |
| ATOM | 198 | CA | SER | A | 28 | 8.239 | 16.802 | 8.582 | 1.00 | 143.12 | A | C |
| ATOM | 199 | CB | SER | A | 28 | 9.212 | 16.816 | 9.756 | 1.00 | 137.44 | A | C |
| ATOM | 200 | OG | SER | A | 28 | 9.563 | 15.501 | 10.133 | 1.00 | 114.04 | A | O |
| ATOM | 201 | C | SER | A | 28 | 7.925 | 18.225 | 8.174 | 1.00 | 130.00 | A | C |
| ATOM | 202 | O | SER | A | 28 | 6.861 | 18.753 | 8.502 | 1.00 | 140.61 | A | O |
| ATOM | 203 | N | THR | A | 29 | 8.856 | 18.844 | 7.458 | 1.00 | 126.10 | A | N |
| ATOM | 204 | CA | THR | A | 29 | 8.627 | 20.183 | 6.950 | 1.00 | 134.92 | A | C |
| ATOM | 205 | CB | THR | A | 29 | 9.751 | 20.638 | 5.995 | 1.00 | 133.90 | A | C |
| ATOM | 206 | OG1 | THR | A | 29 | 9.569 | 20.043 | 4.714 | 1.00 | 128.38 | A | O |
| ATOM | 207 | CG2 | THR | A | 29 | 11.087 | 20.228 | 6.499 | 1.00 | 134.19 | A | C |
| ATOM | 208 | C | THR | A | 29 | 7.291 | 20.254 | 6.205 | 1.00 | 132.98 | A | C |
| ATOM | 209 | O | THR | A | 29 | 6.512 | 21.199 | 6.386 | 1.00 | 146.23 | A | O |
| ATOM | 210 | N | ALA | A | 30 | 7.033 | 19.232 | 5.388 | 1.00 | 116.20 | A | N |
| ATOM | 211 | CA | ALA | A | 30 | 5.927 | 19.259 | 4.444 | 1.00 | 125.18 | A | C |
| ATOM | 212 | CB | ALA | A | 30 | 5.939 | 18.010 | 3.572 | 1.00 | 116.54 | A | C |
| ATOM | 213 | C | ALA | A | 30 | 4.601 | 19.407 | 5.157 | 1.00 | 111.65 | A | C |
| ATOM | 214 | O | ALA | A | 30 | 3.708 | 20.110 | 4.692 | 1.00 | 105.47 | A | O |
| ATOM | 215 | N | VAL | A | 31 | 4.487 | 18.750 | 6.301 | 1.00 | 110.30 | A | N |
| ATOM | 216 | CA | VAL | A | 31 | 3.277 | 18.801 | 7.097 | 1.00 | 108.68 | A | C |
| ATOM | 217 | CB | VAL | A | 31 | 3.546 | 18.407 | 8.558 | 1.00 | 121.92 | A | C |
| ATOM | 218 | CG1 | VAL | A | 31 | 2.276 | 18.493 | 9.390 | 1.00 | 111.49 | A | C |
| ATOM | 219 | CG2 | VAL | A | 31 | 4.106 | 17.002 | 8.625 | 1.00 | 140.12 | A | C |
| ATOM | 220 | C | VAL | A | 31 | 2.710 | 20.201 | 7.090 | 1.00 | 121.06 | A | C |
| ATOM | 221 | O | VAL | A | 31 | 1.559 | 20.410 | 6.704 | 1.00 | 122.45 | A | O |
| ATOM | 222 | N | MET | A | 32 | 3.526 | 21.156 | 7.529 | 1.00 | 124.57 | A | N |
| ATOM | 223 | CA | MET | A | 32 | 3.083 | 22.525 | 7.653 | 1.00 | 128.61 | A | C |
| ATOM | 224 | CB | MET | A | 32 | 4.186 | 23.397 | 8.249 | 1.00 | 150.05 | A | C |
| ATOM | 225 | CG | MET | A | 32 | 3.732 | 24.819 | 8.525 | 1.00 | 151.11 | A | C |
| ATOM | 226 | SD | MET | A | 32 | 2.039 | 24.920 | 9.171 | 1.00 | 179.70 | A | S |
| ATOM | 227 | CE | MET | A | 32 | 2.276 | 24.579 | 10.912 | 1.00 | 144.75 | A | C |
| ATOM | 228 | C | MET | A | 32 | 2.612 | 23.033 | 6.298 | 1.00 | 119.57 | A | C |
| ATOM | 229 | O | MET | A | 32 | 1.479 | 23.497 | 6.161 | 1.00 | 128.38 | A | O |
| ATOM | 230 | N | ASN | A | 33 | 3.461 | 22.879 | 5.290 | 1.00 | 112.53 | A | N |
| ATOM | 231 | CA | ASN | A | 33 | 3.127 | 23.294 | 3.935 | 1.00 | 114.15 | A | C |
| ATOM | 232 | CB | ASN | A | 33 | 4.245 | 22.894 | 2.998 | 1.00 | 108.65 | A | C |
| ATOM | 233 | CG | ASN | A | 33 | 5.544 | 23.574 | 3.339 | 1.00 | 125.34 | A | C |
| ATOM | 234 | OD1 | ASN | A | 33 | 5.754 | 24.729 | 2.979 | 1.00 | 141.10 | A | O |
| ATOM | 235 | ND2 | ASN | A | 33 | 6.425 | 22.867 | 4.038 | 1.00 | 123.66 | A | N |
| ATOM | 236 | C | ASN | A | 33 | 1.797 | 22.731 | 3.447 | 1.00 | 123.37 | A | C |
| ATOM | 237 | O | ASN | A | 33 | 1.097 | 23.375 | 2.679 | 1.00 | 124.29 | A | O |
| ATOM | 238 | N | PHE | A | 34 | 1.455 | 21.530 | 3.909 | 1.00 | 124.07 | A | N |
| ATOM | 239 | CA | PHE | A | 34 | 0.161 | 20.933 | 3.607 | 1.00 | 119.05 | A | C |
| ATOM | 240 | CB | PHE | A | 34 | 0.098 | 19.490 | 4.081 | 1.00 | 123.00 | A | C |
| ATOM | 241 | CG | PHE | A | 34 | 0.591 | 18.499 | 3.080 | 1.00 | 118.43 | A | C |
| ATOM | 242 | CD1 | PHE | A | 34 | −0.114 | 18.269 | 1.920 | 1.00 | 93.58 | A | C |
| ATOM | 243 | CE1 | PHE | A | 34 | 0.349 | 17.346 | 1.015 | 1.00 | 100.64 | A | C |
| ATOM | 244 | CZ | PHE | A | 34 | 1.519 | 16.643 | 1.259 | 1.00 | 100.41 | A | C |
| ATOM | 245 | CE2 | PHE | A | 34 | 2.231 | 16.854 | 2.412 | 1.00 | 96.27 | A | C |
| ATOM | 246 | CD2 | PHE | A | 34 | 1.764 | 17.776 | 3.320 | 1.00 | 120.73 | A | C |
| ATOM | 247 | C | PHE | A | 34 | −0.970 | 21.692 | 4.261 | 1.00 | 120.69 | A | C |
| ATOM | 248 | O | PHE | A | 34 | −1.924 | 22.069 | 3.592 | 1.00 | 119.16 | A | O |
| ATOM | 249 | N | PHE | A | 35 | −0.862 | 21.907 | 5.570 | 1.00 | 123.51 | A | N |
| ATOM | 250 | CA | PHE | A | 35 | −1.911 | 22.591 | 6.324 | 1.00 | 128.76 | A | C |
| ATOM | 251 | CB | PHE | A | 35 | −1.472 | 22.875 | 7.755 | 1.00 | 130.79 | A | C |
| ATOM | 252 | CG | PHE | A | 35 | −1.472 | 21.672 | 8.632 | 1.00 | 129.65 | A | C |
| ATOM | 253 | CD1 | PHE | A | 35 | −2.399 | 20.661 | 8.441 | 1.00 | 135.78 | A | C |
| ATOM | 254 | CE1 | PHE | A | 35 | −2.398 | 19.538 | 9.256 | 1.00 | 141.44 | A | C |
| ATOM | 255 | CZ | PHE | A | 35 | −1.476 | 19.425 | 10.286 | 1.00 | 135.01 | A | C |
| ATOM | 256 | CE2 | PHE | A | 35 | −0.556 | 20.437 | 10.492 | 1.00 | 158.44 | A | C |
| ATOM | 257 | CD2 | PHE | A | 35 | −0.559 | 21.552 | 9.667 | 1.00 | 148.38 | A | C |
| ATOM | 258 | C | PHE | A | 35 | −2.267 | 23.903 | 5.686 | 1.00 | 130.18 | A | C |
| ATOM | 259 | O | PHE | A | 35 | −3.435 | 24.201 | 5.469 | 1.00 | 138.97 | A | O |
| ATOM | 260 | N | VAL | A | 36 | −1.242 | 24.684 | 5.386 | 1.00 | 141.31 | A | N |
| ATOM | 261 | CA | VAL | A | 36 | −1.437 | 26.036 | 4.907 | 1.00 | 147.83 | A | C |
| ATOM | 262 | CB | VAL | A | 36 | −0.153 | 26.873 | 5.051 | 1.00 | 153.82 | A | C |
| ATOM | 263 | CG1 | VAL | A | 36 | −0.259 | 28.162 | 4.244 | 1.00 | 151.82 | A | C |
| ATOM | 264 | CG2 | VAL | A | 36 | 0.121 | 27.160 | 6.525 | 1.00 | 135.68 | A | C |
| ATOM | 265 | C | VAL | A | 36 | −1.950 | 26.027 | 3.475 | 1.00 | 143.48 | A | C |
| ATOM | 266 | O | VAL | A | 36 | −3.038 | 26.543 | 3.211 | 1.00 | 136.00 | A | O |
| ATOM | 267 | N | GLY | A | 37 | −1.172 | 25.432 | 2.567 | 1.00 | 137.90 | A | N |
| ATOM | 268 | CA | GLY | A | 37 | −1.607 | 25.225 | 1.186 | 1.00 | 142.69 | A | C |
| ATOM | 269 | C | GLY | A | 37 | −3.057 | 24.764 | 1.123 | 1.00 | 145.95 | A | C |
| ATOM | 270 | O | GLY | A | 37 | −3.867 | 25.324 | 0.373 | 1.00 | 128.51 | A | O |
| ATOM | 271 | N | GLY | A | 38 | −3.383 | 23.754 | 1.928 | 1.00 | 142.65 | A | N |
| ATOM | 272 | CA | GLY | A | 38 | −4.753 | 23.248 | 2.060 | 1.00 | 134.47 | A | C |
| ATOM | 273 | C | GLY | A | 38 | −5.703 | 24.355 | 2.460 | 1.00 | 120.90 | A | C |
| ATOM | 274 | O | GLY | A | 38 | −6.718 | 24.578 | 1.813 | 1.00 | 120.73 | A | O |
| ATOM | 275 | N | LEU | A | 39 | −5.359 | 25.061 | 3.525 | 1.00 | 116.89 | A | N |
| ATOM | 276 | CA | LEU | A | 39 | −6.154 | 26.182 | 3.978 | 1.00 | 126.65 | A | C |
| ATOM | 277 | CB | LEU | A | 39 | −5.469 | 26.859 | 5.151 | 1.00 | 124.69 | A | C |
| ATOM | 278 | CG | LEU | A | 39 | −6.272 | 27.960 | 5.821 | 1.00 | 150.74 | A | C |
| ATOM | 279 | CD1 | LEU | A | 39 | −7.663 | 27.482 | 6.237 | 1.00 | 172.35 | A | C |
| ATOM | 280 | CD2 | LEU | A | 39 | −5.505 | 28.513 | 7.014 | 1.00 | 144.52 | A | C |
| ATOM | 281 | C | LEU | A | 39 | −6.399 | 27.203 | 2.876 | 1.00 | 121.84 | A | C |
| ATOM | 282 | O | LEU | A | 39 | −7.538 | 27.530 | 2.589 | 1.00 | 121.18 | A | O |
| ATOM | 283 | N | SER | A | 40 | −5.335 | 27.704 | 2.257 | 1.00 | 115.93 | A | N |
| ATOM | 284 | CA | SER | A | 40 | −5.469 | 28.679 | 1.172 | 1.00 | 127.00 | A | C |
| ATOM | 285 | CB | SER | A | 40 | −4.174 | 28.820 | 0.384 | 1.00 | 133.03 | A | C |
| ATOM | 286 | OG | SER | A | 40 | −3.047 | 28.499 | 1.172 | 1.00 | 162.30 | A | O |
| ATOM | 287 | C | SER | A | 40 | −6.529 | 28.232 | 0.199 | 1.00 | 123.90 | A | C |
| ATOM | 288 | O | SER | A | 40 | −7.424 | 28.989 | −0.144 | 1.00 | 141.58 | A | O |
| ATOM | 289 | N | ILE | A | 41 | −6.421 | 26.986 | −0.236 | 1.00 | 116.87 | A | N |
| ATOM | 290 | CA | ILE | A | 41 | −7.170 | 26.499 | −1.376 | 1.00 | 112.73 | A | C |
| ATOM | 291 | CB | ILE | A | 41 | −6.552 | 25.196 | −1.897 | 1.00 | 111.17 | A | C |
| ATOM | 292 | CG1 | ILE | A | 41 | −5.208 | 25.517 | −2.536 | 1.00 | 114.24 | A | C |
| ATOM | 293 | CD1 | ILE | A | 41 | −4.349 | 24.293 | −2.697 | 1.00 | 130.59 | A | C |
| ATOM | 294 | CG2 | ILE | A | 41 | −7.462 | 24.470 | −2.881 | 1.00 | 119.28 | A | C |
| ATOM | 295 | C | ILE | A | 41 | −8.632 | 26.355 | −1.023 | 1.00 | 125.47 | A | C |
| ATOM | 296 | O | ILE | A | 41 | −9.501 | 26.701 | −1.821 | 1.00 | 127.73 | A | O |
| ATOM | 297 | N | VAL | A | 42 | −8.897 | 25.874 | 0.184 | 1.00 | 126.06 | A | N |
| ATOM | 298 | CA | VAL | A | 42 | −10.251 | 25.884 | 0.701 | 1.00 | 138.17 | A | C |
| ATOM | 299 | CB | VAL | A | 42 | −10.338 | 25.367 | 2.156 | 1.00 | 144.07 | A | C |
| ATOM | 300 | CG1 | VAL | A | 42 | −11.795 | 25.175 | 2.551 | 1.00 | 128.74 | A | C |
| ATOM | 301 | CG2 | VAL | A | 42 | −9.586 | 24.060 | 2.336 | 1.00 | 147.14 | A | C |
| ATOM | 302 | C | VAL | A | 42 | −10.788 | 27.310 | 0.657 | 1.00 | 123.89 | A | C |
| ATOM | 303 | O | VAL | A | 42 | −11.814 | 27.568 | 0.047 | 1.00 | 134.60 | A | O |
| ATOM | 304 | N | CYS | A | 43 | −10.080 | 28.236 | 1.293 | 1.00 | 122.79 | A | N |
| ATOM | 305 | CA | CYS | A | 43 | −10.535 | 29.620 | 1.388 | 1.00 | 128.14 | A | C |
| ATOM | 306 | CB | CYS | A | 43 | −9.455 | 30.485 | 2.017 | 1.00 | 125.68 | A | C |
| ATOM | 307 | SG | CYS | A | 43 | −9.109 | 29.981 | 3.703 | 1.00 | 157.34 | A | S |
| ATOM | 308 | C | CYS | A | 43 | −10.923 | 30.200 | 0.043 | 1.00 | 141.59 | A | C |
| ATOM | 309 | O | CYS | A | 43 | −12.072 | 30.597 | −0.171 | 1.00 | 139.08 | A | O |
| ATOM | 310 | N | ASN | A | 44 | −9.963 | 30.225 | −0.870 | 1.00 | 127.83 | A | N |
| ATOM | 311 | CA | ASN | A | 44 | −10.184 | 30.802 | −2.174 | 1.00 | 129.96 | A | C |
| ATOM | 312 | CB | ASN | A | 44 | −8.904 | 30.750 | −2.997 | 1.00 | 154.58 | A | C |
| ATOM | 313 | CG | ASN | A | 44 | −7.804 | 31.623 | −2.419 | 1.00 | 181.76 | A | C |
| ATOM | 314 | OD1 | ASN | A | 44 | −6.625 | 31.411 | −2.700 | 1.00 | 179.64 | A | O |
| ATOM | 315 | ND2 | ASN | A | 44 | −8.186 | 32.613 | −1.597 | 1.00 | 183.85 | A | N |
| ATOM | 316 | C | ASN | A | 44 | −11.333 | 30.138 | −2.909 | 1.00 | 137.62 | A | C |
| ATOM | 317 | O | ASN | A | 44 | −11.919 | 30.738 | −3.800 | 1.00 | 145.67 | A | O |
| ATOM | 318 | N | VAL | A | 45 | −11.675 | 28.918 | −2.508 | 1.00 | 129.90 | A | N |
| ATOM | 319 | CA | VAL | A | 45 | −12.779 | 28.195 | −3.128 | 1.00 | 132.34 | A | C |
| ATOM | 320 | CB | VAL | A | 45 | −12.645 | 26.686 | −2.902 | 1.00 | 124.68 | A | C |
| ATOM | 321 | CG1 | VAL | A | 45 | −14.009 | 26.016 | −2.782 | 1.00 | 124.51 | A | C |
| ATOM | 322 | CG2 | VAL | A | 45 | −11.809 | 26.081 | −4.021 | 1.00 | 134.72 | A | C |
| ATOM | 323 | C | VAL | A | 45 | −14.165 | 28.724 | −2.721 | 1.00 | 138.39 | A | C |
| ATOM | 324 | O | VAL | A | 45 | −15.037 | 28.928 | −3.578 | 1.00 | 133.47 | A | O |
| ATOM | 325 | N | VAL | A | 46 | −14.369 | 28.957 | −1.428 | 1.00 | 121.56 | A | N |
| ATOM | 326 | CA | VAL | A | 46 | −15.570 | 29.665 | −0.985 | 1.00 | 156.39 | A | C |
| ATOM | 327 | CB | VAL | A | 46 | −15.690 | 29.757 | 0.558 | 1.00 | 154.49 | A | C |
| ATOM | 328 | CG1 | VAL | A | 46 | −15.222 | 28.459 | 1.199 | 1.00 | 139.75 | A | C |
| ATOM | 329 | CG2 | VAL | A | 46 | −14.893 | 30.924 | 1.112 | 1.00 | 143.08 | A | C |
| ATOM | 330 | C | VAL | A | 46 | −15.556 | 31.047 | −1.639 | 1.00 | 150.90 | A | C |
| ATOM | 331 | O | VAL | A | 46 | −16.548 | 31.491 | −2.204 | 1.00 | 141.29 | A | O |
| ATOM | 332 | N | VAL | A | 47 | −14.396 | 31.691 | −1.606 | 1.00 | 145.57 | A | N |
| ATOM | 333 | CA | VAL | A | 47 | −14.212 | 33.008 | −2.185 | 1.00 | 141.47 | A | C |
| ATOM | 334 | CB | VAL | A | 47 | −12.793 | 33.508 | −1.909 | 1.00 | 137.07 | A | C |
| ATOM | 335 | CG1 | VAL | A | 47 | −12.486 | 34.739 | −2.730 | 1.00 | 135.06 | A | C |
| ATOM | 336 | CG2 | VAL | A | 47 | −12.612 | 33.786 | −0.428 | 1.00 | 153.44 | A | C |
| ATOM | 337 | C | VAL | A | 47 | −14.493 | 33.021 | −3.689 | 1.00 | 154.04 | A | C |
| ATOM | 338 | O | VAL | A | 47 | −15.112 | 33.946 | −4.195 | 1.00 | 156.29 | A | O |
| ATOM | 339 | N | ILE | A | 48 | −14.034 | 31.997 | −4.401 | 1.00 | 149.77 | A | N |
| ATOM | 340 | CA | ILE | A | 48 | −14.376 | 31.852 | −5.812 | 1.00 | 150.12 | A | C |
| ATOM | 341 | CB | ILE | A | 48 | −13.701 | 30.643 | −6.468 | 1.00 | 155.05 | A | C |
| ATOM | 342 | CG1 | ILE | A | 48 | −12.223 | 30.928 | −6.707 | 1.00 | 168.50 | A | C |
| ATOM | 343 | CD1 | ILE | A | 48 | −11.395 | 29.671 | −6.805 | 1.00 | 159.99 | A | C |
| ATOM | 344 | CG2 | ILE | A | 48 | −14.384 | 30.308 | −7.792 | 1.00 | 162.97 | A | C |
| ATOM | 345 | C | ILE | A | 48 | −15.871 | 31.679 | −5.967 | 1.00 | 159.98 | A | C |
| ATOM | 346 | O | ILE | A | 48 | −16.475 | 32.301 | −6.838 | 1.00 | 158.52 | A | O |
| ATOM | 347 | N | THR | A | 49 | −16.460 | 30.828 | −5.127 | 1.00 | 158.84 | A | N |
| ATOM | 348 | CA | THR | A | 49 | −17.890 | 30.560 | −5.223 | 1.00 | 142.96 | A | C |
| ATOM | 349 | CB | THR | A | 49 | −18.328 | 29.350 | −4.396 | 1.00 | 132.73 | A | C |
| ATOM | 350 | OG1 | THR | A | 49 | −17.938 | 29.545 | −3.032 | 1.00 | 171.55 | A | O |
| ATOM | 351 | CG2 | THR | A | 49 | −17.692 | 28.094 | −4.938 | 1.00 | 102.52 | A | C |
| ATOM | 352 | C | THR | A | 49 | −18.705 | 31.783 | −4.824 | 1.00 | 153.97 | A | C |
| ATOM | 353 | O | THR | A | 49 | −19.770 | 32.003 | −5.363 | 1.00 | 173.10 | A | O |
| ATOM | 354 | N | TYR | A | 50 | −18.186 | 32.589 | −3.904 | 1.00 | 165.86 | A | N |
| ATOM | 355 | CA | TYR | A | 50 | −18.905 | 33.761 | −3.429 | 1.00 | 165.43 | A | C |
| ATOM | 356 | CB | TYR | A | 50 | −18.270 | 34.351 | −2.182 | 1.00 | 175.74 | A | C |
| ATOM | 357 | CG | TYR | A | 50 | −18.829 | 33.753 | −0.935 | 1.00 | 230.91 | A | C |
| ATOM | 358 | CD1 | TYR | A | 50 | −18.243 | 32.615 | −0.376 | 1.00 | 236.49 | A | C |
| ATOM | 359 | CE1 | TYR | A | 50 | −18.754 | 32.043 | 0.779 | 1.00 | 280.12 | A | C |
| ATOM | 360 | CZ | TYR | A | 50 | −19.886 | 32.611 | 1.381 | 1.00 | 324.66 | A | C |
| ATOM | 361 | OH | TYR | A | 50 | −20.418 | 32.063 | 2.532 | 1.00 | 310.38 | A | O |
| ATOM | 362 | CE2 | TYR | A | 50 | −20.498 | 33.729 | 0.825 | 1.00 | 251.84 | A | C |
| ATOM | 363 | CD2 | TYR | A | 50 | −19.966 | 34.298 | −0.323 | 1.00 | 234.71 | A | C |
| ATOM | 364 | C | TYR | A | 50 | −19.034 | 34.850 | −4.448 | 1.00 | 163.29 | A | C |
| ATOM | 365 | O | TYR | A | 50 | −19.984 | 35.625 | −4.388 | 1.00 | 185.24 | A | O |
| ATOM | 366 | N | SER | A | 51 | −18.071 | 34.947 | −5.353 | 1.00 | 156.23 | A | N |
| ATOM | 367 | CA | SER | A | 51 | −18.183 | 35.867 | −6.463 | 1.00 | 183.70 | A | C |
| ATOM | 368 | CB | SER | A | 51 | −16.813 | 36.409 | −6.872 | 1.00 | 195.60 | A | C |
| ATOM | 369 | OG | SER | A | 51 | −15.883 | 35.351 | −7.000 | 1.00 | 207.20 | A | O |
| ATOM | 370 | C | SER | A | 51 | −18.904 | 35.184 | −7.619 | 1.00 | 182.19 | A | C |
| ATOM | 371 | O | SER | A | 51 | −19.679 | 35.812 | −8.337 | 1.00 | 216.12 | A | O |
| ATOM | 372 | N | ALA | A | 52 | −18.672 | 33.888 | −7.780 | 1.00 | 163.06 | A | N |
| ATOM | 373 | CA | ALA | A | 52 | −19.520 | 33.091 | −8.655 | 1.00 | 178.32 | A | C |
| ATOM | 374 | CB | ALA | A | 52 | −19.136 | 31.626 | −8.578 | 1.00 | 162.25 | A | C |
| ATOM | 375 | C | ALA | A | 52 | −20.998 | 33.290 | −8.283 | 1.00 | 192.45 | A | C |
| ATOM | 376 | O | ALA | A | 52 | −21.873 | 33.203 | −9.145 | 1.00 | 208.33 | A | O |
| ATOM | 377 | N | LEU | A | 53 | −21.251 | 33.559 | −6.997 | 1.00 | 191.87 | A | N |
| ATOM | 378 | CA | LEU | A | 53 | −22.580 | 33.932 | −6.494 | 1.00 | 208.85 | A | C |
| ATOM | 379 | CB | LEU | A | 53 | −22.583 | 34.043 | −4.960 | 1.00 | 248.92 | A | C |
| ATOM | 380 | CG | LEU | A | 53 | −23.098 | 32.911 | −4.069 | 1.00 | 242.85 | A | C |
| ATOM | 381 | CD1 | LEU | A | 53 | −22.271 | 32.841 | −2.789 | 1.00 | 190.70 | A | C |
| ATOM | 382 | CD2 | LEU | A | 53 | −24.591 | 33.072 | −3.777 | 1.00 | 315.38 | A | C |
| ATOM | 383 | C | LEU | A | 53 | −23.017 | 35.263 | −7.048 | 1.00 | 207.80 | A | C |
| ATOM | 384 | O | LEU | A | 53 | −24.100 | 35.381 | −7.621 | 1.00 | 209.79 | A | O |
| ATOM | 385 | N | HIS | A | 54 | −22.161 | 36.262 | −6.862 | 1.00 | 211.10 | A | N |
| ATOM | 386 | CA | HIS | A | 54 | −22.528 | 37.640 | −7.117 | 1.00 | 237.71 | A | C |
| ATOM | 387 | CB | HIS | A | 54 | −22.356 | 38.489 | −5.855 | 1.00 | 260.20 | A | C |
| ATOM | 388 | CG | HIS | A | 54 | −23.007 | 37.892 | −4.621 | 1.00 | 268.59 | A | C |
| ATOM | 389 | ND1 | HIS | A | 54 | −22.301 | 37.273 | −3.649 | 1.00 | 232.20 | A | N |
| ATOM | 390 | CE1 | HIS | A | 54 | −23.142 | 36.845 | −2.689 | 1.00 | 248.23 | A | C |
| ATOM | 391 | NE2 | HIS | A | 54 | −24.393 | 37.185 | −3.047 | 1.00 | 280.87 | A | N |
| ATOM | 392 | CD2 | HIS | A | 54 | −24.345 | 37.829 | −4.234 | 1.00 | 293.38 | A | C |
| ATOM | 393 | C | HIS | A | 54 | −21.665 | 38.144 | −8.219 | 1.00 | 254.00 | A | C |
| ATOM | 394 | O | HIS | A | 54 | −20.569 | 38.651 | −7.956 | 1.00 | 237.38 | A | O |
| ATOM | 395 | N | PRO | A | 55 | −22.127 | 37.980 | −9.479 | 1.00 | 280.33 | A | N |
| ATOM | 396 | CA | PRO | A | 55 | −21.378 | 38.435 | −10.656 | 1.00 | 289.10 | A | C |
| ATOM | 397 | CB | PRO | A | 55 | −22.309 | 38.080 | −11.827 | 1.00 | 303.21 | A | C |
| ATOM | 398 | CG | PRO | A | 55 | −23.199 | 36.994 | −11.318 | 1.00 | 230.23 | A | C |
| ATOM | 399 | CD | PRO | A | 55 | −23.372 | 37.277 | −9.853 | 1.00 | 267.16 | A | C |
| ATOM | 400 | C | PRO | A | 55 | −21.096 | 39.944 | −10.618 | 1.00 | 304.75 | A | C |
| ATOM | 401 | O | PRO | A | 55 | −20.850 | 40.548 | −11.658 | 1.00 | 315.84 | A | O |
| ATOM | 402 | N | THR | A | 56 | −21.106 | 40.523 | −9.415 | 1.00 | 309.22 | A | N |
| ATOM | 403 | CA | THR | A | 56 | −21.147 | 41.970 | −9.212 | 1.00 | 302.16 | A | C |
| ATOM | 404 | CB | THR | A | 56 | −19.761 | 42.637 | −9.396 | 1.00 | 237.96 | A | C |
| ATOM | 405 | OG1 | THR | A | 56 | −19.359 | 42.530 | −10.765 | 1.00 | 345.81 | A | O |
| ATOM | 406 | CG2 | THR | A | 56 | −18.700 | 41.990 | −8.506 | 1.00 | 196.02 | A | C |
| ATOM | 407 | C | THR | A | 56 | −22.211 | 42.607 | −10.129 | 1.00 | 306.97 | A | C |
| ATOM | 408 | O | THR | A | 56 | −22.145 | 43.792 | −10.453 | 1.00 | 346.45 | A | O |
| ATOM | 409 | N | ALA | A | 57 | −23.181 | 41.795 | −10.554 | 1.00 | 338.20 | A | N |
| ATOM | 410 | CA | ALA | A | 57 | −24.355 | 42.267 | −11.292 | 1.00 | 335.27 | A | C |
| ATOM | 411 | CB | ALA | A | 57 | −24.893 | 41.182 | −12.218 | 1.00 | 304.22 | A | C |
| ATOM | 412 | C | ALA | A | 57 | −25.448 | 42.744 | −10.329 | 1.00 | 365.36 | A | C |
| ATOM | 413 | O | ALA | A | 57 | −26.266 | 43.604 | −10.712 | 1.00 | 320.43 | A | O |
| ATOM | 414 | N | PRO | A | 58 | −25.478 | 42.176 | −9.089 | 1.00 | 387.74 | A | N |
| ATOM | 415 | CA | PRO | A | 58 | −26.370 | 42.656 | −8.030 | 1.00 | 372.96 | A | C |
| ATOM | 416 | CB | PRO | A | 58 | −26.104 | 41.679 | −6.868 | 1.00 | 265.06 | A | C |
| ATOM | 417 | CG | PRO | A | 58 | −24.767 | 41.070 | −7.153 | 1.00 | 262.65 | A | C |
| ATOM | 418 | CD | PRO | A | 58 | −24.757 | 40.961 | −8.651 | 1.00 | 319.73 | A | C |
| ATOM | 419 | C | PRO | A | 58 | −26.038 | 44.083 | −7.600 | 1.00 | 328.83 | A | C |
| ATOM | 420 | O | PRO | A | 58 | −26.714 | 44.639 | −6.733 | 1.00 | 321.02 | A | O |
| ATOM | 421 | N | SER | A | 74 | −12.165 | 42.455 | −9.780 | 1.00 | 197.82 | A | N |
| ATOM | 422 | CA | SER | A | 74 | −12.824 | 41.154 | −9.981 | 1.00 | 202.13 | A | C |
| ATOM | 423 | CB | SER | A | 74 | −12.453 | 40.529 | −11.344 | 1.00 | 173.61 | A | C |
| ATOM | 424 | OG | SER | A | 74 | −12.125 | 41.490 | −12.323 | 1.00 | 135.39 | A | O |
| ATOM | 425 | C | SER | A | 74 | −12.465 | 40.167 | −8.872 | 1.00 | 171.00 | A | C |
| ATOM | 426 | O | SER | A | 74 | −12.170 | 40.562 | −7.738 | 1.00 | 176.52 | A | O |
| ATOM | 427 | N | PHE | A | 75 | −12.509 | 38.879 | −9.207 | 1.00 | 167.19 | A | N |
| ATOM | 428 | CA | PHE | A | 75 | −11.930 | 37.831 | −8.359 | 1.00 | 166.66 | A | C |
| ATOM | 429 | CB | PHE | A | 75 | −12.632 | 36.488 | −8.575 | 1.00 | 162.43 | A | C |
| ATOM | 430 | CG | PHE | A | 75 | −12.821 | 36.090 | −10.021 | 1.00 | 185.68 | A | C |
| ATOM | 431 | CD1 | PHE | A | 75 | −11.743 | 35.669 | −10.818 | 1.00 | 204.66 | A | C |
| ATOM | 432 | CE1 | PHE | A | 75 | −11.939 | 35.297 | −12.142 | 1.00 | 192.89 | A | C |
| ATOM | 433 | CZ | PHE | A | 75 | −13.220 | 35.307 | −12.672 | 1.00 | 168.34 | A | C |
| ATOM | 434 | CE2 | PHE | A | 75 | −14.300 | 35.686 | −11.885 | 1.00 | 182.85 | A | C |
| ATOM | 435 | CD2 | PHE | A | 75 | −14.100 | 36.068 | −10.569 | 1.00 | 178.46 | A | C |
| ATOM | 436 | C | PHE | A | 75 | −10.409 | 37.685 | −8.547 | 1.00 | 162.88 | A | C |
| ATOM | 437 | O | PHE | A | 75 | −9.793 | 36.665 | −8.196 | 1.00 | 136.58 | A | O |
| ATOM | 438 | N | TYR | A | 76 | −9.811 | 38.723 | −9.117 | 1.00 | 165.03 | A | N |
| ATOM | 439 | CA | TYR | A | 76 | −8.363 | 38.877 | −9.166 | 1.00 | 174.01 | A | C |
| ATOM | 440 | CB | TYR | A | 76 | −8.040 | 40.343 | −9.479 | 1.00 | 198.26 | A | C |
| ATOM | 441 | CG | TYR | A | 76 | −6.620 | 40.791 | −9.201 | 1.00 | 209.25 | A | C |
| ATOM | 442 | CD1 | TYR | A | 76 | −5.603 | 40.594 | −10.144 | 1.00 | 229.03 | A | C |
| ATOM | 443 | CE1 | TYR | A | 76 | −4.307 | 41.025 | −9.896 | 1.00 | 247.22 | A | C |
| ATOM | 444 | CZ | TYR | A | 76 | −4.019 | 41.680 | −8.697 | 1.00 | 240.12 | A | C |
| ATOM | 445 | OH | TYR | A | 76 | −2.741 | 42.120 | −8.435 | 1.00 | 260.02 | A | O |
| ATOM | 446 | CE2 | TYR | A | 76 | −5.014 | 41.890 | −7.754 | 1.00 | 221.13 | A | C |
| ATOM | 447 | CD2 | TYR | A | 76 | −6.305 | 41.454 | −8.014 | 1.00 | 202.82 | A | C |
| ATOM | 448 | C | TYR | A | 76 | −7.704 | 38.446 | −7.853 | 1.00 | 186.44 | A | C |
| ATOM | 449 | O | TYR | A | 76 | −6.566 | 37.972 | −7.854 | 1.00 | 189.07 | A | O |
| ATOM | 450 | N | GLY | A | 77 | −8.427 | 38.629 | −6.745 | 1.00 | 194.24 | A | N |
| ATOM | 451 | CA | GLY | A | 77 | −7.927 | 38.329 | −5.397 | 1.00 | 171.94 | A | C |
| ATOM | 452 | C | GLY | A | 77 | −7.600 | 36.863 | −5.187 | 1.00 | 164.22 | A | C |
| ATOM | 453 | O | GLY | A | 77 | −6.493 | 36.529 | −4.770 | 1.00 | 161.49 | A | O |
| ATOM | 454 | N | PRO | A | 78 | −8.569 | 35.980 | −5.461 | 1.00 | 167.57 | A | N |
| ATOM | 455 | CA | PRO | A | 78 | −8.330 | 34.537 | −5.479 | 1.00 | 167.78 | A | C |
| ATOM | 456 | CB | PRO | A | 78 | −9.623 | 33.977 | −6.063 | 1.00 | 151.63 | A | C |
| ATOM | 457 | CG | PRO | A | 78 | −10.661 | 34.943 | −5.628 | 1.00 | 160.26 | A | C |
| ATOM | 458 | CD | PRO | A | 78 | −10.004 | 36.291 | −5.439 | 1.00 | 169.11 | A | C |
| ATOM | 459 | C | PRO | A | 78 | −7.175 | 34.154 | −6.377 | 1.00 | 152.95 | A | C |
| ATOM | 460 | O | PRO | A | 78 | −6.381 | 33.288 | −6.001 | 1.00 | 157.92 | A | O |
| ATOM | 461 | N | ALA | A | 79 | −7.093 | 34.775 | −7.555 | 1.00 | 153.02 | A | N |
| ATOM | 462 | CA | ALA | A | 79 | −5.984 | 34.537 | −8.476 | 1.00 | 149.96 | A | C |
| ATOM | 463 | CB | ALA | A | 79 | −6.078 | 35.428 | −9.702 | 1.00 | 152.59 | A | C |
| ATOM | 464 | C | ALA | A | 79 | −4.677 | 34.758 | −7.742 | 1.00 | 162.36 | A | C |
| ATOM | 465 | O | ALA | A | 79 | −3.834 | 33.870 | −7.676 | 1.00 | 147.12 | A | O |
| ATOM | 466 | N | THR | A | 80 | −4.532 | 35.939 | −7.157 | 1.00 | 175.53 | A | N |
| ATOM | 467 | CA | THR | A | 80 | −3.372 | 36.253 | −6.333 | 1.00 | 161.34 | A | C |
| ATOM | 468 | CB | THR | A | 80 | −3.388 | 37.720 | −5.869 | 1.00 | 173.95 | A | C |
| ATOM | 469 | OG1 | THR | A | 80 | −4.528 | 37.949 | −5.029 | 1.00 | 194.62 | A | O |
| ATOM | 470 | CG2 | THR | A | 80 | −3.443 | 38.650 | −7.056 | 1.00 | 179.32 | A | C |
| ATOM | 471 | C | THR | A | 80 | −3.300 | 35.338 | −5.106 | 1.00 | 175.40 | A | C |
| ATOM | 472 | O | THR | A | 80 | −2.221 | 34.871 | −4.736 | 1.00 | 167.58 | A | O |
| ATOM | 473 | N | GLY | A | 81 | −4.452 | 35.088 | −4.490 | 1.00 | 166.46 | A | N |
| ATOM | 474 | CA | GLY | A | 81 | −4.509 | 34.276 | −3.284 | 1.00 | 155.01 | A | C |
| ATOM | 475 | C | GLY | A | 81 | −3.863 | 32.919 | −3.451 | 1.00 | 161.20 | A | C |
| ATOM | 476 | O | GLY | A | 81 | −2.799 | 32.645 | −2.873 | 1.00 | 164.44 | A | O |
| ATOM | 477 | N | LEU | A | 82 | −4.500 | 32.078 | −4.268 | 1.00 | 168.41 | A | N |
| ATOM | 478 | CA | LEU | A | 82 | −4.082 | 30.687 | −4.424 | 1.00 | 159.57 | A | C |
| ATOM | 479 | CB | LEU | A | 82 | −5.247 | 29.817 | −4.903 | 1.00 | 133.78 | A | C |
| ATOM | 480 | CG | LEU | A | 82 | −5.843 | 30.025 | −6.288 | 1.00 | 144.40 | A | C |
| ATOM | 481 | CD1 | LEU | A | 82 | −5.211 | 29.029 | −7.238 | 1.00 | 109.34 | A | C |
| ATOM | 482 | CD2 | LEU | A | 82 | −7.329 | 29.752 | −6.225 | 1.00 | 141.80 | A | C |
| ATOM | 483 | C | LEU | A | 82 | −2.851 | 30.572 | −5.325 | 1.00 | 143.61 | A | C |
| ATOM | 484 | O | LEU | A | 82 | −2.345 | 29.475 | −5.608 | 1.00 | 137.05 | A | O |
| ATOM | 485 | N | LEU | A | 83 | −2.364 | 31.719 | −5.762 | 1.00 | 135.19 | A | N |
| ATOM | 486 | CA | LEU | A | 83 | −1.064 | 31.774 | −6.387 | 1.00 | 145.26 | A | C |
| ATOM | 487 | CB | LEU | A | 83 | −0.692 | 33.211 | −6.711 | 1.00 | 134.37 | A | C |
| ATOM | 488 | CG | LEU | A | 83 | 0.513 | 33.288 | −7.622 | 1.00 | 175.50 | A | C |
| ATOM | 489 | CD1 | LEU | A | 83 | 0.193 | 32.653 | −8.973 | 1.00 | 181.99 | A | C |
| ATOM | 490 | CD2 | LEU | A | 83 | 0.925 | 34.745 | −7.757 | 1.00 | 206.17 | A | C |
| ATOM | 491 | C | LEU | A | 83 | −0.000 | 31.131 | −5.500 | 1.00 | 146.57 | A | C |
| ATOM | 492 | O | LEU | A | 83 | 0.621 | 30.141 | −5.888 | 1.00 | 149.56 | A | O |
| ATOM | 493 | N | PHE | A | 84 | 0.203 | 31.686 | −4.310 | 1.00 | 125.67 | A | N |
| ATOM | 494 | CA | PHE | A | 84 | 1.125 | 31.064 | −3.375 | 1.00 | 157.55 | A | C |
| ATOM | 495 | CB | PHE | A | 84 | 1.989 | 32.087 | −2.626 | 1.00 | 161.15 | A | C |
| ATOM | 496 | CG | PHE | A | 84 | 1.245 | 33.271 | −2.113 | 1.00 | 168.59 | A | C |
| ATOM | 497 | CD1 | PHE | A | 84 | 0.690 | 33.253 | −0.839 | 1.00 | 175.91 | A | C |
| ATOM | 498 | CE1 | PHE | A | 84 | 0.009 | 34.356 | −0.348 | 1.00 | 217.88 | A | C |
| ATOM | 499 | CZ | PHE | A | 84 | −0.109 | 35.496 | −1.129 | 1.00 | 232.82 | A | C |
| ATOM | 500 | CE2 | PHE | A | 84 | 0.457 | 35.527 | −2.398 | 1.00 | 204.69 | A | C |
| ATOM | 501 | CD2 | PHE | A | 84 | 1.138 | 34.423 | −2.884 | 1.00 | 158.13 | A | C |
| ATOM | 502 | C | PHE | A | 84 | 0.460 | 30.062 | −2.434 | 1.00 | 162.61 | A | C |
| ATOM | 503 | O | PHE | A | 84 | 1.131 | 29.395 | −1.641 | 1.00 | 166.86 | A | O |
| ATOM | 504 | N | GLY | A | 85 | −0.857 | 29.941 | −2.543 | 1.00 | 156.57 | A | N |
| ATOM | 505 | CA | GLY | A | 85 | −1.551 | 28.774 | −2.009 | 1.00 | 158.97 | A | C |
| ATOM | 506 | C | GLY | A | 85 | −0.936 | 27.490 | −2.549 | 1.00 | 153.04 | A | C |
| ATOM | 507 | O | GLY | A | 85 | −0.547 | 26.610 | −1.775 | 1.00 | 135.79 | A | O |
| ATOM | 508 | N | PHE | A | 86 | −0.832 | 27.401 | −3.877 | 1.00 | 132.83 | A | N |
| ATOM | 509 | CA | PHE | A | 86 | −0.226 | 26.258 | −4.540 | 1.00 | 123.33 | A | C |
| ATOM | 510 | CB | PHE | A | 86 | −0.314 | 26.401 | −6.052 | 1.00 | 120.12 | A | C |
| ATOM | 511 | CG | PHE | A | 86 | −1.661 | 26.096 | −6.598 | 1.00 | 113.20 | A | C |
| ATOM | 512 | CD1 | PHE | A | 86 | −2.567 | 25.342 | −5.857 | 1.00 | 119.95 | A | C |
| ATOM | 513 | CE1 | PHE | A | 86 | −3.822 | 25.043 | −6.364 | 1.00 | 118.00 | A | C |
| ATOM | 514 | CZ | PHE | A | 86 | −4.185 | 25.490 | −7.629 | 1.00 | 107.65 | A | C |
| ATOM | 515 | CE2 | PHE | A | 86 | −3.282 | 26.235 | −8.379 | 1.00 | 126.15 | A | C |
| ATOM | 516 | CD2 | PHE | A | 86 | −2.027 | 26.530 | −7.863 | 1.00 | 124.54 | A | C |
| ATOM | 517 | C | PHE | A | 86 | 1.220 | 26.107 | −4.158 | 1.00 | 116.96 | A | C |
| ATOM | 518 | O | PHE | A | 86 | 1.680 | 25.008 | −3.912 | 1.00 | 115.25 | A | O |
| ATOM | 519 | N | THR | A | 87 | 1.932 | 27.223 | −4.104 | 1.00 | 122.59 | A | N |
| ATOM | 520 | CA | THR | A | 87 | 3.354 | 27.197 | −3.828 | 1.00 | 126.92 | A | C |
| ATOM | 521 | CB | THR | A | 87 | 3.908 | 28.605 | −3.590 | 1.00 | 141.51 | A | C |
| ATOM | 522 | OG1 | THR | A | 87 | 3.773 | 29.369 | −4.798 | 1.00 | 160.76 | A | O |
| ATOM | 523 | CG2 | THR | A | 87 | 5.391 | 28.531 | −3.159 | 1.00 | 131.88 | A | C |
| ATOM | 524 | C | THR | A | 87 | 3.667 | 26.341 | −2.624 | 1.00 | 122.46 | A | C |
| ATOM | 525 | O | THR | A | 87 | 4.657 | 25.616 | −2.615 | 1.00 | 130.38 | A | O |
| ATOM | 526 | N | TYR | A | 88 | 2.816 | 26.418 | −1.611 | 1.00 | 119.53 | A | N |
| ATOM | 527 | CA | TYR | A | 88 | 3.037 | 25.660 | −0.397 | 1.00 | 111.61 | A | C |
| ATOM | 528 | CB | TYR | A | 88 | 2.210 | 26.241 | 0.730 | 1.00 | 137.05 | A | C |
| ATOM | 529 | CG | TYR | A | 88 | 2.634 | 27.642 | 1.089 | 1.00 | 149.13 | A | C |
| ATOM | 530 | CD1 | TYR | A | 88 | 3.951 | 27.915 | 1.476 | 1.00 | 133.00 | A | C |
| ATOM | 531 | CE1 | TYR | A | 88 | 4.352 | 29.200 | 1.809 | 1.00 | 168.96 | A | C |
| ATOM | 532 | CZ | TYR | A | 88 | 3.439 | 30.232 | 1.755 | 1.00 | 194.66 | A | C |
| ATOM | 533 | OH | TYR | A | 88 | 3.835 | 31.520 | 2.091 | 1.00 | 207.47 | A | O |
| ATOM | 534 | CE2 | TYR | A | 88 | 2.122 | 29.985 | 1.369 | 1.00 | 182.23 | A | C |
| ATOM | 535 | CD2 | TYR | A | 88 | 1.729 | 28.694 | 1.042 | 1.00 | 159.01 | A | C |
| ATOM | 536 | C | TYR | A | 88 | 2.709 | 24.218 | −0.615 | 1.00 | 107.58 | A | C |
| ATOM | 537 | O | TYR | A | 88 | 3.504 | 23.343 | −0.298 | 1.00 | 116.97 | A | O |
| ATOM | 538 | N | LEU | A | 89 | 1.533 | 23.981 | −1.181 | 1.00 | 119.21 | A | N |
| ATOM | 539 | CA | LEU | A | 89 | 1.113 | 22.649 | −1.570 | 1.00 | 109.42 | A | C |
| ATOM | 540 | CB | LEU | A | 89 | −0.302 | 22.694 | −2.148 | 1.00 | 105.36 | A | C |
| ATOM | 541 | CG | LEU | A | 89 | −0.950 | 21.365 | −2.496 | 1.00 | 98.54 | A | C |
| ATOM | 542 | CD1 | LEU | A | 89 | −0.771 | 20.392 | −1.365 | 1.00 | 112.06 | A | C |
| ATOM | 543 | CD2 | LEU | A | 89 | −2.423 | 21.535 | −2.735 | 1.00 | 99.15 | A | C |
| ATOM | 544 | C | LEU | A | 89 | 2.106 | 22.006 | −2.547 | 1.00 | 112.56 | A | C |
| ATOM | 545 | O | LEU | A | 89 | 2.401 | 20.821 | −2.440 | 1.00 | 107.24 | A | O |
| ATOM | 546 | N | TYR | A | 90 | 2.624 | 22.792 | −3.484 | 1.00 | 106.81 | A | N |
| ATOM | 547 | CA | TYR | A | 90 | 3.692 | 22.352 | −4.369 | 1.00 | 115.56 | A | C |
| ATOM | 548 | CB | TYR | A | 90 | 4.160 | 23.483 | −5.301 | 1.00 | 115.05 | A | C |
| ATOM | 549 | CG | TYR | A | 90 | 4.529 | 23.005 | −6.692 | 1.00 | 128.38 | A | C |
| ATOM | 550 | CD1 | TYR | A | 90 | 5.526 | 22.048 | −6.895 | 1.00 | 128.66 | A | C |
| ATOM | 551 | CE1 | TYR | A | 90 | 5.851 | 21.601 | −8.175 | 1.00 | 151.57 | A | C |
| ATOM | 552 | CZ | TYR | A | 90 | 5.174 | 22.103 | −9.267 | 1.00 | 152.79 | A | C |
| ATOM | 553 | OH | TYR | A | 90 | 5.491 | 21.660 | −10.535 | 1.00 | 184.57 | A | O |
| ATOM | 554 | CE2 | TYR | A | 90 | 4.186 | 23.051 | −9.089 | 1.00 | 156.69 | A | C |
| ATOM | 555 | CD2 | TYR | A | 90 | 3.867 | 23.497 | −7.812 | 1.00 | 150.44 | A | C |
| ATOM | 556 | C | TYR | A | 90 | 4.875 | 21.870 | −3.545 | 1.00 | 121.14 | A | C |
| ATOM | 557 | O | TYR | A | 90 | 5.435 | 20.811 | −3.822 | 1.00 | 123.89 | A | O |
| ATOM | 558 | N | ALA | A | 91 | 5.256 | 22.653 | −2.540 | 1.00 | 108.83 | A | N |
| ATOM | 559 | CA | ALA | A | 91 | 6.351 | 22.277 | −1.674 | 1.00 | 112.70 | A | C |
| ATOM | 560 | CB | ALA | A | 91 | 6.589 | 23.346 | −0.651 | 1.00 | 105.51 | A | C |
| ATOM | 561 | C | ALA | A | 91 | 6.024 | 20.983 | −0.970 | 1.00 | 109.62 | A | C |
| ATOM | 562 | O | ALA | A | 91 | 6.789 | 20.021 | −1.028 | 1.00 | 123.54 | A | O |
| ATOM | 563 | N | ALA | A | 92 | 4.877 | 20.962 | −0.307 | 1.00 | 112.24 | A | N |
| ATOM | 564 | CA | ALA | A | 92 | 4.500 | 19.837 | 0.519 | 1.00 | 111.05 | A | C |
| ATOM | 565 | CB | ALA | A | 92 | 3.078 | 20.010 | 1.022 | 1.00 | 104.90 | A | C |
| ATOM | 566 | C | ALA | A | 92 | 4.648 | 18.565 | −0.286 | 1.00 | 112.99 | A | C |
| ATOM | 567 | O | ALA | A | 92 | 5.297 | 17.601 | 0.153 | 1.00 | 109.87 | A | O |
| ATOM | 568 | N | ILE | A | 93 | 4.078 | 18.588 | −1.483 | 1.00 | 99.98 | A | N |
| ATOM | 569 | CA | ILE | A | 93 | 4.048 | 17.402 | −2.310 | 1.00 | 107.21 | A | C |
| ATOM | 570 | CB | ILE | A | 93 | 3.036 | 17.551 | −3.442 | 1.00 | 96.25 | A | C |
| ATOM | 571 | CG1 | ILE | A | 93 | 1.633 | 17.398 | −2.853 | 1.00 | 99.91 | A | C |
| ATOM | 572 | CD1 | ILE | A | 93 | 0.515 | 17.581 | −3.856 | 1.00 | 130.95 | A | C |
| ATOM | 573 | CG2 | ILE | A | 93 | 3.295 | 16.530 | −4.541 | 1.00 | 109.19 | A | C |
| ATOM | 574 | C | ILE | A | 93 | 5.435 | 17.022 | −2.816 | 1.00 | 116.92 | A | C |
| ATOM | 575 | O | ILE | A | 93 | 5.850 | 15.874 | −2.686 | 1.00 | 115.47 | A | O |
| ATOM | 576 | N | ASN | A | 94 | 6.161 | 17.990 | −3.364 | 1.00 | 124.67 | A | N |
| ATOM | 577 | CA | ASN | A | 94 | 7.536 | 17.745 | −3.827 | 1.00 | 134.75 | A | C |
| ATOM | 578 | CB | ASN | A | 94 | 8.156 | 19.011 | −4.425 | 1.00 | 138.54 | A | C |
| ATOM | 579 | CG | ASN | A | 94 | 8.028 | 19.084 | −5.944 | 1.00 | 140.95 | A | C |
| ATOM | 580 | OD1 | ASN | A | 94 | 7.451 | 18.205 | −6.585 | 1.00 | 141.32 | A | O |
| ATOM | 581 | ND2 | ASN | A | 94 | 8.572 | 20.140 | −6.530 | 1.00 | 145.16 | A | N |
| ATOM | 582 | C | ASN | A | 94 | 8.445 | 17.144 | −2.742 | 1.00 | 125.82 | A | C |
| ATOM | 583 | O | ASN | A | 94 | 9.349 | 16.358 | −3.048 | 1.00 | 123.46 | A | O |
| ATOM | 584 | N | HIS | A | 95 | 8.185 | 17.507 | −1.483 | 1.00 | 125.90 | A | N |
| ATOM | 585 | CA | HIS | A | 95 | 8.955 | 17.000 | −0.342 | 1.00 | 106.62 | A | C |
| ATOM | 586 | CB | HIS | A | 95 | 8.764 | 17.872 | 0.873 | 1.00 | 97.70 | A | C |
| ATOM | 587 | CG | HIS | A | 95 | 9.828 | 18.895 | 1.033 | 1.00 | 124.24 | A | C |
| ATOM | 588 | ND1 | HIS | A | 95 | 9.563 | 20.147 | 1.435 | 1.00 | 146.85 | A | N |
| ATOM | 589 | CE1 | HIS | A | 95 | 10.708 | 20.849 | 1.480 | 1.00 | 155.98 | A | C |
| ATOM | 590 | NE2 | HIS | A | 95 | 11.716 | 20.046 | 1.086 | 1.00 | 217.03 | A | N |
| ATOM | 591 | CD2 | HIS | A | 95 | 11.209 | 18.831 | 0.802 | 1.00 | 137.13 | A | C |
| ATOM | 592 | C | HIS | A | 95 | 8.591 | 15.618 | 0.054 | 1.00 | 117.88 | A | C |
| ATOM | 593 | O | HIS | A | 95 | 9.460 | 14.757 | 0.196 | 1.00 | 126.78 | A | O |
| ATOM | 594 | N | THR | A | 96 | 7.302 | 15.405 | 0.285 | 1.00 | 116.99 | A | N |
| ATOM | 595 | CA | THR | A | 96 | 6.848 | 14.111 | 0.753 | 1.00 | 109.61 | A | C |
| ATOM | 596 | CB | THR | A | 96 | 5.345 | 14.095 | 1.070 | 1.00 | 109.01 | A | C |
| ATOM | 597 | OG1 | THR | A | 96 | 4.656 | 14.835 | 0.065 | 1.00 | 117.28 | A | O |
| ATOM | 598 | CG2 | THR | A | 96 | 5.091 | 14.764 | 2.369 | 1.00 | 100.56 | A | C |
| ATOM | 599 | C | THR | A | 96 | 7.175 | 13.053 | −0.279 | 1.00 | 121.34 | A | C |
| ATOM | 600 | O | THR | A | 96 | 7.607 | 11.963 | 0.090 | 1.00 | 124.64 | A | O |
| ATOM | 601 | N | PHE | A | 97 | 7.022 | 13.388 | −1.560 | 1.00 | 101.47 | A | N |
| ATOM | 602 | CA | PHE | A | 97 | 7.224 | 12.404 | −2.615 | 1.00 | 100.07 | A | C |
| ATOM | 603 | CB | PHE | A | 97 | 6.186 | 12.564 | −3.720 | 1.00 | 99.04 | A | C |
| ATOM | 604 | CG | PHE | A | 97 | 4.818 | 12.146 | −3.291 | 1.00 | 115.62 | A | C |
| ATOM | 605 | CD1 | PHE | A | 97 | 4.057 | 12.987 | −2.502 | 1.00 | 119.44 | A | C |
| ATOM | 606 | CE1 | PHE | A | 97 | 2.799 | 12.612 | −2.074 | 1.00 | 126.86 | A | C |
| ATOM | 607 | CZ | PHE | A | 97 | 2.300 | 11.363 | −2.407 | 1.00 | 123.39 | A | C |
| ATOM | 608 | CE2 | PHE | A | 97 | 3.063 | 10.497 | −3.176 | 1.00 | 115.13 | A | C |
| ATOM | 609 | CD2 | PHE | A | 97 | 4.323 | 10.882 | −3.603 | 1.00 | 113.44 | A | C |
| ATOM | 610 | C | PHE | A | 97 | 8.629 | 12.337 | −3.150 | 1.00 | 98.35 | A | C |
| ATOM | 611 | O | PHE | A | 97 | 8.974 | 11.388 | −3.829 | 1.00 | 103.10 | A | O |
| ATOM | 612 | N | GLY | A | 98 | 9.434 | 13.333 | −2.801 | 1.00 | 113.80 | A | N |
| ATOM | 613 | CA | GLY | A | 98 | 10.865 | 13.338 | −3.115 | 1.00 | 134.88 | A | C |
| ATOM | 614 | C | GLY | A | 98 | 11.053 | 13.559 | −4.595 | 1.00 | 118.48 | A | C |
| ATOM | 615 | O | GLY | A | 98 | 11.549 | 12.688 | −5.308 | 1.00 | 135.04 | A | O |
| ATOM | 616 | N | LEU | A | 99 | 10.663 | 14.740 | −5.053 | 1.00 | 132.80 | A | N |
| ATOM | 617 | CA | LEU | A | 99 | 10.573 | 15.020 | −6.479 | 1.00 | 125.22 | A | C |
| ATOM | 618 | CB | LEU | A | 99 | 9.144 | 15.453 | −6.816 | 1.00 | 127.28 | A | C |
| ATOM | 619 | CG | LEU | A | 99 | 8.008 | 14.470 | −6.535 | 1.00 | 100.76 | A | C |
| ATOM | 620 | CD1 | LEU | A | 99 | 6.694 | 15.213 | −6.404 | 1.00 | 107.73 | A | C |
| ATOM | 621 | CD2 | LEU | A | 99 | 7.873 | 13.437 | −7.623 | 1.00 | 97.95 | A | C |
| ATOM | 622 | C | LEU | A | 99 | 11.583 | 16.085 | −6.940 | 1.00 | 130.98 | A | C |
| ATOM | 623 | O | LEU | A | 99 | 12.341 | 16.632 | −6.138 | 1.00 | 157.55 | A | O |
| ATOM | 624 | N | ASP | A | 100 | 11.578 | 16.369 | −8.241 | 1.00 | 157.54 | A | N |
| ATOM | 625 | CA | ASP | A | 100 | 12.470 | 17.354 | −8.842 | 1.00 | 161.45 | A | C |
| ATOM | 626 | CB | ASP | A | 100 | 12.579 | 17.100 | −10.346 | 1.00 | 172.31 | A | C |
| ATOM | 627 | CG | ASP | A | 100 | 14.007 | 17.077 | −10.839 | 1.00 | 187.34 | A | C |
| ATOM | 628 | OD1 | ASP | A | 100 | 14.810 | 17.938 | −10.408 | 1.00 | 187.23 | A | O |
| ATOM | 629 | OD2 | ASP | A | 100 | 14.318 | 16.189 | −11.666 | 1.00 | 180.28 | A | O |
| ATOM | 630 | C | ASP | A | 100 | 11.990 | 18.780 | −8.596 | 1.00 | 168.55 | A | C |
| ATOM | 631 | O | ASP | A | 100 | 10.819 | 19.127 | −8.823 | 1.00 | 154.54 | A | O |
| ATOM | 632 | N | TRP | A | 101 | 12.918 | 19.615 | −8.153 | 1.00 | 149.23 | A | N |
| ATOM | 633 | CA | TRP | A | 101 | 12.592 | 20.990 | −7.822 | 1.00 | 164.01 | A | C |
| ATOM | 634 | CB | TRP | A | 101 | 13.490 | 21.475 | −6.693 | 1.00 | 164.48 | A | C |
| ATOM | 635 | CG | TRP | A | 101 | 13.180 | 20.818 | −5.390 | 1.00 | 178.62 | A | C |
| ATOM | 636 | CD1 | TRP | A | 101 | 13.875 | 19.782 | −4.778 | 1.00 | 216.55 | A | C |
| ATOM | 637 | NE1 | TRP | A | 101 | 13.280 | 19.433 | −3.594 | 1.00 | 270.41 | A | N |
| ATOM | 638 | CE2 | TRP | A | 101 | 12.171 | 20.169 | −3.380 | 1.00 | 117.61 | A | C |
| ATOM | 639 | CD2 | TRP | A | 101 | 12.050 | 21.101 | −4.505 | 1.00 | 149.39 | A | C |
| ATOM | 640 | CE3 | TRP | A | 101 | 10.995 | 22.002 | −4.526 | 1.00 | 143.56 | A | C |
| ATOM | 641 | CZ3 | TRP | A | 101 | 10.083 | 21.991 | −3.462 | 1.00 | 155.47 | A | C |
| ATOM | 642 | CH2 | TRP | A | 101 | 10.216 | 21.091 | −2.384 | 1.00 | 145.06 | A | C |
| ATOM | 643 | CZ2 | TRP | A | 101 | 11.266 | 20.166 | −2.322 | 1.00 | 160.85 | A | C |
| ATOM | 644 | C | TRP | A | 101 | 12.670 | 21.932 | −8.996 | 1.00 | 165.00 | A | C |
| ATOM | 645 | O | TRP | A | 101 | 12.591 | 23.152 | −8.823 | 1.00 | 160.47 | A | O |
| ATOM | 646 | N | ARG | A | 102 | 12.820 | 21.387 | −10.200 | 1.00 | 143.00 | A | N |
| ATOM | 647 | CA | ARG | A | 102 | 12.898 | 22.208 | −11.396 | 1.00 | 142.94 | A | C |
| ATOM | 648 | CB | ARG | A | 102 | 13.428 | 21.396 | −12.583 | 1.00 | 151.31 | A | C |
| ATOM | 649 | CG | ARG | A | 102 | 14.942 | 21.330 | −12.661 | 1.00 | 169.29 | A | C |
| ATOM | 650 | CD | ARG | A | 102 | 15.430 | 19.915 | −12.942 | 1.00 | 172.38 | A | C |
| ATOM | 651 | NE | ARG | A | 102 | 15.408 | 19.540 | −14.359 | 1.00 | 207.19 | A | N |
| ATOM | 652 | CZ | ARG | A | 102 | 15.448 | 18.286 | −14.817 | 1.00 | 198.92 | A | C |
| ATOM | 653 | NH1 | ARG | A | 102 | 15.480 | 17.256 | −13.981 | 1.00 | 217.80 | A | N |
| ATOM | 654 | NH2 | ARG | A | 102 | 15.447 | 18.055 | −16.126 | 1.00 | 201.69 | A | N |
| ATOM | 655 | C | ARG | A | 102 | 11.528 | 22.822 | −11.692 | 1.00 | 149.41 | A | C |
| ATOM | 656 | O | ARG | A | 102 | 11.379 | 24.051 | −11.647 | 1.00 | 158.67 | A | O |
| ATOM | 657 | N | PRO | A | 103 | 10.518 | 21.975 | −11.969 | 1.00 | 135.34 | A | N |
| ATOM | 658 | CA | PRO | A | 103 | 9.195 | 22.486 | −12.275 | 1.00 | 148.33 | A | C |
| ATOM | 659 | CB | PRO | A | 103 | 8.368 | 21.208 | −12.413 | 1.00 | 135.64 | A | C |
| ATOM | 660 | CG | PRO | A | 103 | 9.335 | 20.214 | −12.933 | 1.00 | 127.64 | A | C |
| ATOM | 661 | CD | PRO | A | 103 | 10.567 | 20.509 | −12.132 | 1.00 | 151.30 | A | C |
| ATOM | 662 | C | PRO | A | 103 | 8.652 | 23.384 | −11.168 | 1.00 | 143.53 | A | C |
| ATOM | 663 | O | PRO | A | 103 | 7.907 | 24.318 | −11.444 | 1.00 | 140.04 | A | O |
| ATOM | 664 | N | TYR | A | 104 | 9.034 | 23.109 | −9.928 | 1.00 | 138.87 | A | N |
| ATOM | 665 | CA | TYR | A | 104 | 8.732 | 24.024 | −8.851 | 1.00 | 141.23 | A | C |
| ATOM | 666 | CB | TYR | A | 104 | 9.169 | 23.439 | −7.518 | 1.00 | 131.96 | A | C |
| ATOM | 667 | CG | TYR | A | 104 | 8.978 | 24.389 | −6.355 | 1.00 | 141.50 | A | C |
| ATOM | 668 | CD1 | TYR | A | 104 | 7.744 | 24.489 | −5.712 | 1.00 | 128.47 | A | C |
| ATOM | 669 | CE1 | TYR | A | 104 | 7.564 | 25.352 | −4.645 | 1.00 | 112.66 | A | C |
| ATOM | 670 | CZ | TYR | A | 104 | 8.619 | 26.131 | −4.198 | 1.00 | 154.25 | A | C |
| ATOM | 671 | OH | TYR | A | 104 | 8.411 | 26.983 | −3.133 | 1.00 | 176.37 | A | O |
| ATOM | 672 | CE2 | TYR | A | 104 | 9.861 | 26.050 | −4.818 | 1.00 | 167.08 | A | C |
| ATOM | 673 | CD2 | TYR | A | 104 | 10.030 | 25.191 | −5.898 | 1.00 | 137.53 | A | C |
| ATOM | 674 | C | TYR | A | 104 | 9.419 | 25.371 | −9.044 | 1.00 | 151.49 | A | C |
| ATOM | 675 | O | TYR | A | 104 | 8.802 | 26.419 | −8.854 | 1.00 | 161.17 | A | O |
| ATOM | 676 | N | SER | A | 105 | 10.704 | 25.341 | −9.390 | 1.00 | 130.68 | A | N |
| ATOM | 677 | CA | SER | A | 105 | 11.503 | 26.561 | −9.420 | 1.00 | 137.72 | A | C |
| ATOM | 678 | CB | SER | A | 105 | 12.985 | 26.233 | −9.480 | 1.00 | 177.91 | A | C |
| ATOM | 679 | OG | SER | A | 105 | 13.273 | 25.427 | −10.613 | 1.00 | 167.44 | A | O |
| ATOM | 680 | C | SER | A | 105 | 11.126 | 27.456 | −10.590 | 1.00 | 159.30 | A | C |
| ATOM | 681 | O | SER | A | 105 | 11.107 | 28.684 | −10.448 | 1.00 | 177.52 | A | O |
| ATOM | 682 | N | TRP | A | 106 | 10.831 | 26.845 | −11.740 | 1.00 | 153.44 | A | N |
| ATOM | 683 | CA | TRP | A | 106 | 10.252 | 27.585 | −12.862 | 1.00 | 158.71 | A | C |
| ATOM | 684 | CB | TRP | A | 106 | 9.955 | 26.672 | −14.047 | 1.00 | 128.25 | A | C |
| ATOM | 685 | CG | TRP | A | 106 | 11.060 | 26.567 | −15.078 | 1.00 | 142.09 | A | C |
| ATOM | 686 | CD1 | TRP | A | 106 | 11.579 | 25.401 | −15.629 | 1.00 | 163.60 | A | C |
| ATOM | 687 | NE1 | TRP | A | 106 | 12.562 | 25.682 | −16.551 | 1.00 | 177.00 | A | N |
| ATOM | 688 | CE2 | TRP | A | 106 | 12.759 | 27.007 | −16.666 | 1.00 | 171.18 | A | C |
| ATOM | 689 | CD2 | TRP | A | 106 | 11.815 | 27.659 | −15.741 | 1.00 | 150.67 | A | C |
| ATOM | 690 | CE3 | TRP | A | 106 | 11.805 | 29.045 | −15.666 | 1.00 | 145.25 | A | C |
| ATOM | 691 | CZ3 | TRP | A | 106 | 12.710 | 29.766 | −16.476 | 1.00 | 194.23 | A | C |
| ATOM | 692 | CH2 | TRP | A | 106 | 13.613 | 29.121 | −17.345 | 1.00 | 203.05 | A | C |
| ATOM | 693 | CZ2 | TRP | A | 106 | 13.651 | 27.735 | −17.464 | 1.00 | 160.73 | A | C |
| ATOM | 694 | C | TRP | A | 106 | 8.992 | 28.284 | −12.407 | 1.00 | 157.63 | A | C |
| ATOM | 695 | O | TRP | A | 106 | 8.860 | 29.496 | −12.572 | 1.00 | 176.56 | A | O |
| ATOM | 696 | N | TYR | A | 107 | 8.065 | 27.532 | −11.811 | 1.00 | 129.97 | A | N |
| ATOM | 697 | CA | TYR | A | 107 | 6.848 | 28.108 | −11.234 | 1.00 | 141.51 | A | C |
| ATOM | 698 | CB | TYR | A | 107 | 6.075 | 27.039 | −10.447 | 1.00 | 142.08 | A | C |
| ATOM | 699 | CG | TYR | A | 107 | 4.809 | 27.535 | −9.743 | 1.00 | 177.22 | A | C |
| ATOM | 700 | CD1 | TYR | A | 107 | 3.599 | 27.668 | −10.439 | 1.00 | 141.37 | A | C |
| ATOM | 701 | CE1 | TYR | A | 107 | 2.446 | 28.110 | −9.801 | 1.00 | 149.88 | A | C |
| ATOM | 702 | CZ | TYR | A | 107 | 2.489 | 28.424 | −8.445 | 1.00 | 156.43 | A | C |
| ATOM | 703 | OH | TYR | A | 107 | 1.357 | 28.874 | −7.808 | 1.00 | 145.52 | A | O |
| ATOM | 704 | CE2 | TYR | A | 107 | 3.670 | 28.298 | −7.733 | 1.00 | 145.99 | A | C |
| ATOM | 705 | CD2 | TYR | A | 107 | 4.818 | 27.856 | −8.376 | 1.00 | 154.08 | A | C |
| ATOM | 706 | C | TYR | A | 107 | 7.143 | 29.328 | −10.351 | 1.00 | 147.46 | A | C |
| ATOM | 707 | O | TYR | A | 107 | 6.490 | 30.365 | −10.471 | 1.00 | 156.83 | A | O |
| ATOM | 708 | N | SER | A | 108 | 8.136 | 29.204 | −9.479 | 1.00 | 154.28 | A | N |
| ATOM | 709 | CA | SER | A | 108 | 8.488 | 30.284 | −8.563 | 1.00 | 171.81 | A | C |
| ATOM | 710 | CB | SER | A | 108 | 9.627 | 29.845 | −7.637 | 1.00 | 185.68 | A | C |
| ATOM | 711 | OG | SER | A | 108 | 9.158 | 28.972 | −6.618 | 1.00 | 184.78 | A | O |
| ATOM | 712 | C | SER | A | 108 | 8.835 | 31.588 | −9.299 | 1.00 | 175.66 | A | C |
| ATOM | 713 | O | SER | A | 108 | 8.471 | 32.682 | −8.835 | 1.00 | 171.99 | A | O |
| ATOM | 714 | N | LEU | A | 109 | 9.528 | 31.456 | −10.439 | 1.00 | 187.03 | A | N |
| ATOM | 715 | CA | LEU | A | 109 | 9.823 | 32.591 | −11.335 | 1.00 | 197.52 | A | C |
| ATOM | 716 | CB | LEU | A | 109 | 10.747 | 32.175 | −12.496 | 1.00 | 195.09 | A | C |
| ATOM | 717 | CG | LEU | A | 109 | 10.738 | 33.083 | −13.736 | 1.00 | 172.37 | A | C |
| ATOM | 718 | CD1 | LEU | A | 109 | 11.787 | 34.176 | −13.606 | 1.00 | 155.35 | A | C |
| ATOM | 719 | CD2 | LEU | A | 109 | 10.905 | 32.284 | −15.022 | 1.00 | 187.61 | A | C |
| ATOM | 720 | C | LEU | A | 109 | 8.540 | 33.193 | −11.904 | 1.00 | 180.50 | A | C |
| ATOM | 721 | O | LEU | A | 109 | 8.301 | 34.399 | −11.785 | 1.00 | 173.92 | A | O |
| ATOM | 722 | N | PHE | A | 110 | 7.737 | 32.346 | −12.540 | 1.00 | 163.63 | A | N |
| ATOM | 723 | CA | PHE | A | 110 | 6.453 | 32.768 | −13.044 | 1.00 | 155.30 | A | C |
| ATOM | 724 | CB | PHE | A | 110 | 5.604 | 31.559 | −13.440 | 1.00 | 176.97 | A | C |
| ATOM | 725 | CG | PHE | A | 110 | 4.171 | 31.911 | −13.743 | 1.00 | 182.19 | A | C |
| ATOM | 726 | CD1 | PHE | A | 110 | 3.276 | 32.211 | −12.712 | 1.00 | 187.24 | A | C |
| ATOM | 727 | CE1 | PHE | A | 110 | 1.962 | 32.552 | −12.987 | 1.00 | 185.42 | A | C |
| ATOM | 728 | CZ | PHE | A | 110 | 1.522 | 32.593 | −14.301 | 1.00 | 187.73 | A | C |
| ATOM | 729 | CE2 | PHE | A | 110 | 2.395 | 32.284 | −15.336 | 1.00 | 198.69 | A | C |
| ATOM | 730 | CD2 | PHE | A | 110 | 3.714 | 31.960 | −15.056 | 1.00 | 187.73 | A | C |
| ATOM | 731 | C | PHE | A | 110 | 5.708 | 33.614 | −12.005 | 1.00 | 153.42 | A | C |
| ATOM | 732 | O | PHE | A | 110 | 5.164 | 34.675 | −12.334 | 1.00 | 152.40 | A | O |
| ATOM | 733 | N | VAL | A | 111 | 5.680 | 33.141 | −10.762 | 1.00 | 142.26 | A | N |
| ATOM | 734 | CA | VAL | A | 111 | 5.026 | 33.875 | −9.685 | 1.00 | 148.17 | A | C |
| ATOM | 735 | CB | VAL | A | 111 | 5.121 | 33.136 | −8.338 | 1.00 | 170.59 | A | C |
| ATOM | 736 | CG1 | VAL | A | 111 | 4.673 | 34.039 | −7.177 | 1.00 | 146.31 | A | C |
| ATOM | 737 | CG2 | VAL | A | 111 | 4.264 | 31.884 | −8.393 | 1.00 | 177.64 | A | C |
| ATOM | 738 | C | VAL | A | 111 | 5.595 | 35.277 | −9.549 | 1.00 | 162.92 | A | C |
| ATOM | 739 | O | VAL | A | 111 | 4.848 | 36.260 | −9.582 | 1.00 | 167.11 | A | O |
| ATOM | 740 | N | ALA | A | 112 | 6.914 | 35.367 | −9.414 | 1.00 | 158.16 | A | N |
| ATOM | 741 | CA | ALA | A | 112 | 7.581 | 36.661 | −9.332 | 1.00 | 166.78 | A | C |
| ATOM | 742 | CB | ALA | A | 112 | 9.080 | 36.469 | −9.225 | 1.00 | 161.25 | A | C |
| ATOM | 743 | C | ALA | A | 112 | 7.230 | 37.571 | −10.520 | 1.00 | 180.83 | A | C |
| ATOM | 744 | O | ALA | A | 112 | 6.941 | 38.754 | −10.332 | 1.00 | 176.48 | A | O |
| ATOM | 745 | N | ILE | A | 113 | 7.231 | 37.007 | −11.730 | 1.00 | 178.48 | A | N |
| ATOM | 746 | CA | ILE | A | 113 | 6.850 | 37.746 | −12.942 | 1.00 | 185.46 | A | C |
| ATOM | 747 | CB | ILE | A | 113 | 6.878 | 36.847 | −14.206 | 1.00 | 174.16 | A | C |
| ATOM | 748 | CG1 | ILE | A | 113 | 8.279 | 36.267 | −14.430 | 1.00 | 151.93 | A | C |
| ATOM | 749 | CD1 | ILE | A | 113 | 8.382 | 35.339 | −15.621 | 1.00 | 150.13 | A | C |
| ATOM | 750 | CG2 | ILE | A | 113 | 6.440 | 37.624 | −15.441 | 1.00 | 163.23 | A | C |
| ATOM | 751 | C | ILE | A | 113 | 5.468 | 38.395 | −12.782 | 1.00 | 169.36 | A | C |
| ATOM | 752 | O | ILE | A | 113 | 5.192 | 39.442 | −13.354 | 1.00 | 158.63 | A | O |
| ATOM | 753 | N | ASN | A | 114 | 4.603 | 37.780 | −11.989 | 1.00 | 165.89 | A | N |
| ATOM | 754 | CA | ASN | A | 114 | 3.272 | 38.324 | −11.784 | 1.00 | 167.15 | A | C |
| ATOM | 755 | CB | ASN | A | 114 | 2.210 | 37.224 | −11.872 | 1.00 | 172.05 | A | C |
| ATOM | 756 | CG | ASN | A | 114 | 2.207 | 36.520 | −13.214 | 1.00 | 153.90 | A | C |
| ATOM | 757 | OD1 | ASN | A | 114 | 1.150 | 36.141 | −13.725 | 1.00 | 177.61 | A | O |
| ATOM | 758 | ND2 | ASN | A | 114 | 3.392 | 36.337 | −13.791 | 1.00 | 165.28 | A | N |
| ATOM | 759 | C | ASN | A | 114 | 3.137 | 39.103 | −10.482 | 1.00 | 169.27 | A | C |
| ATOM | 760 | O | ASN | A | 114 | 2.068 | 39.619 | −10.184 | 1.00 | 196.72 | A | O |
| ATOM | 761 | N | THR | A | 115 | 4.218 | 39.184 | −9.711 | 1.00 | 178.64 | A | N |
| ATOM | 762 | CA | THR | A | 115 | 4.232 | 40.025 | −8.515 | 1.00 | 164.58 | A | C |
| ATOM | 763 | CB | THR | A | 115 | 5.211 | 39.507 | −7.457 | 1.00 | 188.26 | A | C |
| ATOM | 764 | OG1 | THR | A | 115 | 6.555 | 39.653 | −7.934 | 1.00 | 206.86 | A | O |
| ATOM | 765 | CG2 | THR | A | 115 | 4.929 | 38.051 | −7.138 | 1.00 | 194.55 | A | C |
| ATOM | 766 | C | THR | A | 115 | 4.603 | 41.462 | −8.857 | 1.00 | 185.11 | A | C |
| ATOM | 767 | O | THR | A | 115 | 4.346 | 42.386 | −8.076 | 1.00 | 209.89 | A | O |
| ATOM | 768 | N | VAL | A | 116 | 5.231 | 41.634 | −10.019 | 1.00 | 205.56 | A | N |
| ATOM | 769 | CA | VAL | A | 116 | 5.602 | 42.951 | −10.541 | 1.00 | 200.40 | A | C |
| ATOM | 770 | CB | VAL | A | 116 | 6.419 | 42.834 | −11.848 | 1.00 | 185.42 | A | C |
| ATOM | 771 | CG1 | VAL | A | 116 | 6.587 | 44.189 | −12.501 | 1.00 | 164.56 | A | C |
| ATOM | 772 | CG2 | VAL | A | 116 | 7.770 | 42.197 | −11.576 | 1.00 | 157.66 | A | C |
| ATOM | 773 | C | VAL | A | 116 | 4.345 | 43.784 | −10.783 | 1.00 | 193.06 | A | C |
| ATOM | 774 | O | VAL | A | 116 | 4.196 | 44.865 | −10.191 | 1.00 | 199.11 | A | O |
| ATOM | 775 | N | PRO | A | 117 | 3.432 | 43.285 | −11.653 | 1.00 | 201.76 | A | N |
| ATOM | 776 | CA | PRO | A | 117 | 2.197 | 44.015 | −11.929 | 1.00 | 196.10 | A | C |
| ATOM | 777 | CB | PRO | A | 117 | 1.546 | 43.207 | −13.058 | 1.00 | 184.55 | A | C |
| ATOM | 778 | CG | PRO | A | 117 | 2.613 | 42.324 | −13.613 | 1.00 | 184.87 | A | C |
| ATOM | 779 | CD | PRO | A | 117 | 3.531 | 42.057 | −12.466 | 1.00 | 188.01 | A | C |
| ATOM | 780 | C | PRO | A | 117 | 1.298 | 44.066 | −10.693 | 1.00 | 184.10 | A | C |
| ATOM | 781 | O | PRO | A | 117 | 0.494 | 44.990 | −10.539 | 1.00 | 169.78 | A | O |
| ATOM | 782 | N | ALA | A | 118 | 1.451 | 43.090 | −9.809 | 1.00 | 197.38 | A | N |
| ATOM | 783 | CA | ALA | A | 118 | 0.746 | 43.099 | −8.533 | 1.00 | 204.52 | A | C |
| ATOM | 784 | CB | ALA | A | 118 | 0.986 | 41.803 | −7.785 | 1.00 | 202.57 | A | C |
| ATOM | 785 | C | ALA | A | 118 | 1.172 | 44.288 | −7.689 | 1.00 | 189.93 | A | C |
| ATOM | 786 | O | ALA | A | 118 | 0.330 | 44.971 | −7.113 | 1.00 | 209.06 | A | O |
| ATOM | 787 | N | ALA | A | 119 | 2.475 | 44.541 | −7.643 | 1.00 | 160.78 | A | N |
| ATOM | 788 | CA | ALA | A | 119 | 3.016 | 45.664 | −6.900 | 1.00 | 173.35 | A | C |
| ATOM | 789 | CB | ALA | A | 119 | 4.529 | 45.591 | −6.888 | 1.00 | 180.67 | A | C |
| ATOM | 790 | C | ALA | A | 119 | 2.534 | 47.000 | −7.480 | 1.00 | 203.29 | A | C |
| ATOM | 791 | O | ALA | A | 119 | 1.993 | 47.839 | −6.745 | 1.00 | 178.53 | A | O |
| ATOM | 792 | N | ILE | A | 120 | 2.730 | 47.177 | −8.794 | 1.00 | 219.18 | A | N |
| ATOM | 793 | CA | ILE | A | 120 | 2.202 | 48.322 | −9.552 | 1.00 | 207.35 | A | C |
| ATOM | 794 | CB | ILE | A | 120 | 2.299 | 48.094 | −11.091 | 1.00 | 182.60 | A | C |
| ATOM | 795 | CG1 | ILE | A | 120 | 3.753 | 48.182 | −11.562 | 1.00 | 175.01 | A | C |
| ATOM | 796 | CD1 | ILE | A | 120 | 3.990 | 47.662 | −12.969 | 1.00 | 182.37 | A | C |
| ATOM | 797 | CG2 | ILE | A | 120 | 1.432 | 49.086 | −11.865 | 1.00 | 185.88 | A | C |
| ATOM | 798 | C | ILE | A | 120 | 0.750 | 48.607 | −9.150 | 1.00 | 218.98 | A | C |
| ATOM | 799 | O | ILE | A | 120 | 0.425 | 49.699 | −8.681 | 1.00 | 208.56 | A | O |
| ATOM | 800 | N | LEU | A | 121 | −0.107 | 47.602 | −9.310 | 1.00 | 202.77 | A | N |
| ATOM | 801 | CA | LEU | A | 121 | −1.536 | 47.766 | −9.109 | 1.00 | 208.55 | A | C |
| ATOM | 802 | CB | LEU | A | 121 | −2.307 | 46.577 | −9.735 | 1.00 | 206.45 | A | C |
| ATOM | 803 | CG | LEU | A | 121 | −3.751 | 46.829 | −10.243 | 1.00 | 250.90 | A | C |
| ATOM | 804 | CD1 | LEU | A | 121 | −4.004 | 48.309 | −10.645 | 1.00 | 182.56 | A | C |
| ATOM | 805 | CD2 | LEU | A | 121 | −4.166 | 45.837 | −11.335 | 1.00 | 267.90 | A | C |
| ATOM | 806 | C | LEU | A | 121 | −1.880 | 47.971 | −7.627 | 1.00 | 212.73 | A | C |
| ATOM | 807 | O | LEU | A | 121 | −2.886 | 48.602 | −7.295 | 1.00 | 173.98 | A | O |
| ATOM | 808 | N | SER | A | 122 | −1.028 | 47.464 | −6.740 | 1.00 | 217.29 | A | N |
| ATOM | 809 | CA | SER | A | 122 | −1.323 | 47.477 | −5.302 | 1.00 | 211.14 | A | C |
| ATOM | 810 | CB | SER | A | 122 | −0.628 | 46.318 | −4.579 | 1.00 | 175.63 | A | C |
| ATOM | 811 | OG | SER | A | 122 | 0.775 | 46.384 | −4.747 | 1.00 | 184.23 | A | O |
| ATOM | 812 | C | SER | A | 122 | −0.963 | 48.798 | −4.648 | 1.00 | 185.26 | A | C |
| ATOM | 813 | O | SER | A | 122 | −1.433 | 49.101 | −3.555 | 1.00 | 195.05 | A | O |
| ATOM | 814 | N | HIS | A | 123 | −0.132 | 49.577 | −5.330 | 1.00 | 207.92 | A | N |
| ATOM | 815 | CA | HIS | A | 123 | 0.236 | 50.894 | −4.848 | 1.00 | 230.56 | A | C |
| ATOM | 816 | CB | HIS | A | 123 | 1.583 | 51.320 | −5.424 | 1.00 | 239.21 | A | C |
| ATOM | 817 | CG | HIS | A | 123 | 2.032 | 52.682 | −4.956 | 1.00 | 241.70 | A | C |
| ATOM | 818 | ND1 | HIS | A | 123 | 2.365 | 52.927 | −3.675 | 1.00 | 223.34 | A | N |
| ATOM | 819 | CE1 | HIS | A | 123 | 2.702 | 54.218 | −3.534 | 1.00 | 234.30 | A | C |
| ATOM | 820 | NE2 | HIS | A | 123 | 2.588 | 54.812 | −4.740 | 1.00 | 259.43 | A | N |
| ATOM | 821 | CD2 | HIS | A | 123 | 2.166 | 53.892 | −5.639 | 1.00 | 252.89 | A | C |
| ATOM | 822 | C | HIS | A | 123 | −0.812 | 51.943 | −5.138 | 1.00 | 254.27 | A | C |
| ATOM | 823 | O | HIS | A | 123 | −0.834 | 52.991 | −4.491 | 1.00 | 261.23 | A | O |
| ATOM | 824 | N | TYR | A | 124 | −1.686 | 51.675 | −6.108 | 1.00 | 242.24 | A | N |
| ATOM | 825 | CA | TYR | A | 124 | −2.722 | 52.633 | −6.511 | 1.00 | 236.62 | A | C |
| ATOM | 826 | CB | TYR | A | 124 | −2.856 | 52.696 | −8.038 | 1.00 | 217.76 | A | C |
| ATOM | 827 | CG | TYR | A | 124 | −1.574 | 53.002 | −8.777 | 1.00 | 243.43 | A | C |
| ATOM | 828 | CD1 | TYR | A | 124 | −0.549 | 53.747 | −8.169 | 1.00 | 241.70 | A | C |
| ATOM | 829 | CE1 | TYR | A | 124 | 0.625 | 54.037 | −8.845 | 1.00 | 239.37 | A | C |
| ATOM | 830 | CZ | TYR | A | 124 | 0.779 | 53.596 | −10.156 | 1.00 | 254.60 | A | C |
| ATOM | 831 | OH | TYR | A | 124 | 1.947 | 53.881 | −10.828 | 1.00 | 273.95 | A | O |
| ATOM | 832 | CE2 | TYR | A | 124 | −0.225 | 52.867 | −10.785 | 1.00 | 246.05 | A | C |
| ATOM | 833 | CD2 | TYR | A | 124 | −1.394 | 52.568 | −10.095 | 1.00 | 230.37 | A | C |
| ATOM | 834 | C | TYR | A | 124 | −4.080 | 52.314 | −5.886 | 1.00 | 245.41 | A | C |
| ATOM | 835 | O | TYR | A | 124 | −5.129 | 52.457 | −6.529 | 1.00 | 265.01 | A | O |
| ATOM | 836 | N | SER | A | 125 | −4.054 | 51.873 | −4.633 | 1.00 | 231.09 | A | N |
| ATOM | 837 | CA | SER | A | 125 | −5.272 | 51.523 | −3.910 | 1.00 | 212.56 | A | C |
| ATOM | 838 | CB | SER | A | 125 | −5.513 | 49.999 | −3.963 | 1.00 | 275.48 | A | C |
| ATOM | 839 | OG | SER | A | 125 | −5.883 | 49.531 | −5.262 | 1.00 | 250.64 | A | O |
| ATOM | 840 | C | SER | A | 125 | −5.166 | 51.968 | −2.448 | 1.00 | 258.95 | A | C |
| ATOM | 841 | O | SER | A | 125 | −4.077 | 52.289 | −1.955 | 1.00 | 242.30 | A | O |
| ATOM | 842 | N | ASP | A | 126 | −6.313 | 51.996 | −1.772 | 1.00 | 294.48 | A | N |
| ATOM | 843 | CA | ASP | A | 126 | −6.372 | 51.933 | −0.309 | 1.00 | 286.03 | A | C |
| ATOM | 844 | CB | ASP | A | 126 | −6.023 | 50.502 | 0.173 | 1.00 | 305.46 | A | C |
| ATOM | 845 | CG | ASP | A | 126 | −6.933 | 49.415 | −0.407 | 1.00 | 326.54 | A | C |
| ATOM | 846 | OD1 | ASP | A | 126 | −8.023 | 49.744 | −0.925 | 1.00 | 327.63 | A | O |
| ATOM | 847 | OD2 | ASP | A | 126 | −6.545 | 48.223 | −0.331 | 1.00 | 274.19 | A | O |
| ATOM | 848 | C | ASP | A | 126 | −5.479 | 52.942 | 0.435 | 1.00 | 250.05 | A | C |
| ATOM | 849 | O | ASP | A | 126 | −4.759 | 52.558 | 1.356 | 1.00 | 260.55 | A | O |
| ATOM | 850 | N | MET | A | 127 | −5.504 | 54.216 | 0.038 | 1.00 | 215.42 | A | N |
| ATOM | 851 | CA | MET | A | 127 | −4.876 | 55.272 | 0.853 | 1.00 | 239.53 | A | C |
| ATOM | 852 | CB | MET | A | 127 | −4.535 | 56.497 | 0.007 | 1.00 | 224.51 | A | C |
| ATOM | 853 | CG | MET | A | 127 | −3.574 | 56.178 | −1.131 | 1.00 | 249.46 | A | C |
| ATOM | 854 | SD | MET | A | 127 | −4.023 | 57.038 | −2.648 | 1.00 | 315.42 | A | S |
| ATOM | 855 | CE | MET | A | 127 | −3.505 | 55.896 | −3.927 | 1.00 | 280.28 | A | C |
| ATOM | 856 | C | MET | A | 127 | −5.806 | 55.625 | 2.020 | 1.00 | 237.48 | A | C |
| ATOM | 857 | O | MET | A | 127 | −6.840 | 56.274 | 1.844 | 1.00 | 247.35 | A | O |
| ATOM | 858 | N | LEU | A | 128 | −5.427 | 55.186 | 3.217 | 1.00 | 260.19 | A | N |
| ATOM | 859 | CA | LEU | A | 128 | −6.386 | 55.059 | 4.310 | 1.00 | 241.30 | A | C |
| ATOM | 860 | CB | LEU | A | 128 | −6.271 | 53.672 | 4.964 | 1.00 | 248.50 | A | C |
| ATOM | 861 | CG | LEU | A | 128 | −6.508 | 52.487 | 4.022 | 1.00 | 205.82 | A | C |
| ATOM | 862 | CD1 | LEU | A | 128 | −6.286 | 51.166 | 4.729 | 1.00 | 198.91 | A | C |
| ATOM | 863 | CD2 | LEU | A | 128 | −7.882 | 52.552 | 3.364 | 1.00 | 169.47 | A | C |
| ATOM | 864 | C | LEU | A | 128 | −6.318 | 56.175 | 5.339 | 1.00 | 237.00 | A | C |
| ATOM | 865 | O | LEU | A | 128 | −7.355 | 56.659 | 5.804 | 1.00 | 237.62 | A | O |
| ATOM | 866 | N | ASP | A | 129 | −5.104 | 56.600 | 5.674 | 1.00 | 238.31 | A | N |
| ATOM | 867 | CA | ASP | A | 129 | −4.902 | 57.784 | 6.516 | 1.00 | 276.57 | A | C |
| ATOM | 868 | CB | ASP | A | 129 | −5.804 | 58.960 | 6.063 | 1.00 | 322.48 | A | C |
| ATOM | 869 | CG | ASP | A | 129 | −5.538 | 59.432 | 4.612 | 1.00 | 299.56 | A | C |
| ATOM | 870 | OD1 | ASP | A | 129 | −4.485 | 59.111 | 4.020 | 1.00 | 253.39 | A | O |
| ATOM | 871 | OD2 | ASP | A | 129 | −6.406 | 60.150 | 4.059 | 1.00 | 298.88 | A | O |
| ATOM | 872 | C | ASP | A | 129 | −5.171 | 57.518 | 8.009 | 1.00 | 298.12 | A | C |
| ATOM | 873 | O | ASP | A | 129 | −5.027 | 58.422 | 8.834 | 1.00 | 318.07 | A | O |
| ATOM | 874 | N | ASP | A | 130 | −5.551 | 56.292 | 8.358 | 1.00 | 297.19 | A | N |
| ATOM | 875 | CA | ASP | A | 130 | −6.157 | 56.027 | 9.664 | 1.00 | 324.63 | A | C |
| ATOM | 876 | CB | ASP | A | 130 | −7.571 | 55.463 | 9.469 | 1.00 | 338.72 | A | C |
| ATOM | 877 | CG | ASP | A | 130 | −8.583 | 56.536 | 9.048 | 1.00 | 343.38 | A | C |
| ATOM | 878 | OD1 | ASP | A | 130 | −8.247 | 57.745 | 9.077 | 1.00 | 289.71 | A | O |
| ATOM | 879 | OD2 | ASP | A | 130 | −9.731 | 56.167 | 8.697 | 1.00 | 350.35 | A | O |
| ATOM | 880 | C | ASP | A | 130 | −5.357 | 55.116 | 10.585 | 1.00 | 331.43 | A | C |
| ATOM | 881 | O | ASP | A | 130 | −5.874 | 54.626 | 11.588 | 1.00 | 293.06 | A | O |
| ATOM | 882 | N | HIS | A | 131 | −4.085 | 54.914 | 10.266 | 1.00 | 326.74 | A | N |
| ATOM | 883 | CA | HIS | A | 131 | −3.291 | 53.869 | 10.913 | 1.00 | 301.88 | A | C |
| ATOM | 884 | CB | HIS | A | 131 | −2.478 | 53.097 | 9.874 | 1.00 | 283.81 | A | C |
| ATOM | 885 | CG | HIS | A | 131 | −3.316 | 52.462 | 8.792 | 1.00 | 245.11 | A | C |
| ATOM | 886 | ND1 | HIS | A | 131 | −4.352 | 53.099 | 8.208 | 1.00 | 255.69 | A | N |
| ATOM | 887 | CE1 | HIS | A | 131 | −4.915 | 52.292 | 7.300 | 1.00 | 249.40 | A | C |
| ATOM | 888 | NE2 | HIS | A | 131 | −4.239 | 51.143 | 7.290 | 1.00 | 263.10 | A | N |
| ATOM | 889 | CD2 | HIS | A | 131 | −3.246 | 51.213 | 8.203 | 1.00 | 244.39 | A | C |
| ATOM | 890 | C | HIS | A | 131 | −2.378 | 54.387 | 11.981 | 1.00 | 314.43 | A | C |
| ATOM | 891 | O | HIS | A | 131 | −1.871 | 55.507 | 11.892 | 1.00 | 310.05 | A | O |
| ATOM | 892 | N | LYS | A | 132 | −2.163 | 53.569 | 13.009 | 1.00 | 322.43 | A | N |
| ATOM | 893 | CA | LYS | A | 132 | −1.051 | 53.776 | 13.925 | 1.00 | 332.38 | A | C |
| ATOM | 894 | CB | LYS | A | 132 | −1.332 | 53.190 | 15.323 | 1.00 | 277.63 | A | C |
| ATOM | 895 | CG | LYS | A | 132 | −1.847 | 54.226 | 16.323 | 1.00 | 268.06 | A | C |
| ATOM | 896 | CD | LYS | A | 132 | −2.090 | 53.641 | 17.715 | 1.00 | 266.67 | A | C |
| ATOM | 897 | CE | LYS | A | 132 | −1.646 | 54.579 | 18.844 | 1.00 | 304.19 | A | C |
| ATOM | 898 | NZ | LYS | A | 132 | −2.224 | 55.963 | 18.875 | 1.00 | 312.32 | A | N |
| ATOM | 899 | C | LYS | A | 132 | 0.237 | 53.237 | 13.274 | 1.00 | 335.69 | A | C |
| ATOM | 900 | O | LYS | A | 132 | 1.323 | 53.329 | 13.855 | 1.00 | 352.69 | A | O |
| ATOM | 901 | N | VAL | A | 133 | 0.112 | 52.705 | 12.053 | 1.00 | 305.28 | A | N |
| ATOM | 902 | CA | VAL | A | 133 | 1.279 | 52.436 | 11.204 | 1.00 | 288.41 | A | C |
| ATOM | 903 | CB | VAL | A | 133 | 0.887 | 51.947 | 9.784 | 1.00 | 231.72 | A | C |
| ATOM | 904 | CG1 | VAL | A | 133 | 0.765 | 53.102 | 8.795 | 1.00 | 274.13 | A | C |
| ATOM | 905 | CG2 | VAL | A | 133 | 1.900 | 50.938 | 9.286 | 1.00 | 224.99 | A | C |
| ATOM | 906 | C | VAL | A | 133 | 2.184 | 53.684 | 11.196 | 1.00 | 279.68 | A | C |
| ATOM | 907 | O | VAL | A | 133 | 1.720 | 54.801 | 11.412 | 1.00 | 282.46 | A | O |
| ATOM | 908 | N | LEU | A | 134 | 3.472 | 53.484 | 10.950 | 1.00 | 268.94 | A | N |
| ATOM | 909 | CA | LEU | A | 134 | 4.509 | 54.414 | 11.419 | 1.00 | 240.41 | A | C |
| ATOM | 910 | CB | LEU | A | 134 | 5.888 | 53.761 | 11.343 | 1.00 | 251.61 | A | C |
| ATOM | 911 | CG | LEU | A | 134 | 6.121 | 52.521 | 12.209 | 1.00 | 273.17 | A | C |
| ATOM | 912 | CD1 | LEU | A | 134 | 5.214 | 51.350 | 11.841 | 1.00 | 241.61 | A | C |
| ATOM | 913 | CD2 | LEU | A | 134 | 7.580 | 52.107 | 12.115 | 1.00 | 278.15 | A | C |
| ATOM | 914 | C | LEU | A | 134 | 4.536 | 55.798 | 10.773 | 1.00 | 284.22 | A | C |
| ATOM | 915 | O | LEU | A | 134 | 3.848 | 56.047 | 9.777 | 1.00 | 288.07 | A | O |
| ATOM | 916 | N | GLY | A | 135 | 5.349 | 56.683 | 11.360 | 1.00 | 307.18 | A | N |
| ATOM | 917 | CA | GLY | A | 135 | 5.382 | 58.121 | 11.044 | 1.00 | 289.21 | A | C |
| ATOM | 918 | C | GLY | A | 135 | 6.196 | 58.536 | 9.831 | 1.00 | 319.46 | A | C |
| ATOM | 919 | O | GLY | A | 135 | 6.256 | 59.729 | 9.491 | 1.00 | 400.80 | A | O |
| ATOM | 920 | N | ILE | A | 136 | 6.842 | 57.564 | 9.187 | 1.00 | 376.91 | A | N |
| ATOM | 921 | CA | ILE | A | 136 | 7.456 | 57.805 | 7.882 | 1.00 | 366.80 | A | C |
| ATOM | 922 | CB | ILE | A | 136 | 8.811 | 57.073 | 7.701 | 1.00 | 298.81 | A | C |
| ATOM | 923 | CG1 | ILE | A | 136 | 9.660 | 57.103 | 8.985 | 1.00 | 260.56 | A | C |
| ATOM | 924 | CD1 | ILE | A | 136 | 10.601 | 55.917 | 9.134 | 1.00 | 146.25 | A | C |
| ATOM | 925 | CG2 | ILE | A | 136 | 9.573 | 57.691 | 6.533 | 1.00 | 243.26 | A | C |
| ATOM | 926 | C | ILE | A | 136 | 6.473 | 57.381 | 6.774 | 1.00 | 354.61 | A | C |
| ATOM | 927 | O | ILE | A | 136 | 6.449 | 56.219 | 6.356 | 1.00 | 336.01 | A | O |
| ATOM | 928 | N | THR | A | 137 | 5.660 | 58.330 | 6.307 | 1.00 | 344.27 | A | N |
| ATOM | 929 | CA | THR | A | 137 | 4.675 | 58.093 | 5.224 | 1.00 | 328.80 | A | C |
| ATOM | 930 | CB | THR | A | 137 | 5.299 | 58.290 | 3.817 | 1.00 | 279.86 | A | C |
| ATOM | 931 | OG1 | THR | A | 137 | 6.519 | 57.542 | 3.717 | 1.00 | 300.71 | A | O |
| ATOM | 932 | CG2 | THR | A | 137 | 5.586 | 59.766 | 3.559 | 1.00 | 272.57 | A | C |
| ATOM | 933 | C | THR | A | 137 | 3.896 | 56.754 | 5.329 | 1.00 | 295.08 | A | C |
| ATOM | 934 | O | THR | A | 137 | 3.107 | 56.574 | 6.255 | 1.00 | 268.12 | A | O |
| ATOM | 935 | N | GLU | A | 138 | 4.123 | 55.825 | 4.399 | 1.00 | 261.93 | A | N |
| ATOM | 936 | CA | GLU | A | 138 | 3.465 | 54.502 | 4.416 | 1.00 | 245.12 | A | C |
| ATOM | 937 | CB | GLU | A | 138 | 3.543 | 53.871 | 5.801 | 1.00 | 246.59 | A | C |
| ATOM | 938 | CG | GLU | A | 138 | 4.949 | 53.526 | 6.221 | 1.00 | 228.43 | A | C |
| ATOM | 939 | CD | GLU | A | 138 | 5.095 | 53.517 | 7.720 | 1.00 | 264.30 | A | C |
| ATOM | 940 | OE1 | GLU | A | 138 | 4.443 | 52.673 | 8.365 | 1.00 | 270.37 | A | O |
| ATOM | 941 | OE2 | GLU | A | 138 | 5.857 | 54.351 | 8.250 | 1.00 | 289.84 | A | O |
| ATOM | 942 | C | GLU | A | 138 | 2.012 | 54.504 | 3.924 | 1.00 | 232.58 | A | C |
| ATOM | 943 | O | GLU | A | 138 | 1.150 | 53.795 | 4.463 | 1.00 | 205.83 | A | O |
| ATOM | 944 | N | GLY | A | 139 | 1.753 | 55.309 | 2.904 | 1.00 | 231.52 | A | N |
| ATOM | 945 | CA | GLY | A | 139 | 0.501 | 55.228 | 2.172 | 1.00 | 254.23 | A | C |
| ATOM | 946 | C | GLY | A | 139 | 0.521 | 53.927 | 1.405 | 1.00 | 219.82 | A | C |
| ATOM | 947 | O | GLY | A | 139 | 1.144 | 53.845 | 0.347 | 1.00 | 209.94 | A | O |
| ATOM | 948 | N | ASP | A | 140 | −0.123 | 52.904 | 1.972 | 1.00 | 253.95 | A | N |
| ATOM | 949 | CA | ASP | A | 140 | −0.297 | 51.579 | 1.338 | 1.00 | 252.30 | A | C |
| ATOM | 950 | CB | ASP | A | 140 | −1.446 | 51.610 | 0.324 | 1.00 | 231.60 | A | C |
| ATOM | 951 | CG | ASP | A | 140 | −1.799 | 50.233 | −0.193 | 1.00 | 234.11 | A | C |
| ATOM | 952 | OD1 | ASP | A | 140 | −2.297 | 49.400 | 0.601 | 1.00 | 239.81 | A | O |
| ATOM | 953 | OD2 | ASP | A | 140 | −1.577 | 49.992 | −1.398 | 1.00 | 225.10 | A | O |
| ATOM | 954 | C | ASP | A | 140 | 0.959 | 50.961 | 0.697 | 1.00 | 232.22 | A | C |
| ATOM | 955 | O | ASP | A | 140 | 1.085 | 50.890 | −0.531 | 1.00 | 218.62 | A | O |
| ATOM | 956 | N | TRP | A | 141 | 1.867 | 50.485 | 1.539 | 1.00 | 231.88 | A | N |
| ATOM | 957 | CA | TRP | A | 141 | 3.098 | 49.849 | 1.070 | 1.00 | 213.27 | A | C |
| ATOM | 958 | CB | TRP | A | 141 | 4.253 | 50.112 | 2.035 | 1.00 | 200.96 | A | C |
| ATOM | 959 | CG | TRP | A | 141 | 3.939 | 49.875 | 3.497 | 1.00 | 232.59 | A | C |
| ATOM | 960 | CD1 | TRP | A | 141 | 2.926 | 50.458 | 4.254 | 1.00 | 236.53 | A | C |
| ATOM | 961 | NE1 | TRP | A | 141 | 2.964 | 50.031 | 5.554 | 1.00 | 236.28 | A | N |
| ATOM | 962 | CE2 | TRP | A | 141 | 3.983 | 49.167 | 5.743 | 1.00 | 240.92 | A | C |
| ATOM | 963 | CD2 | TRP | A | 141 | 4.670 | 49.018 | 4.447 | 1.00 | 230.41 | A | C |
| ATOM | 964 | CE3 | TRP | A | 141 | 5.784 | 48.189 | 4.366 | 1.00 | 244.61 | A | C |
| ATOM | 965 | CZ3 | TRP | A | 141 | 6.192 | 47.503 | 5.522 | 1.00 | 297.68 | A | C |
| ATOM | 966 | CH2 | TRP | A | 141 | 5.517 | 47.654 | 6.748 | 1.00 | 315.09 | A | C |
| ATOM | 967 | CZ2 | TRP | A | 141 | 4.403 | 48.492 | 6.882 | 1.00 | 286.85 | A | C |
| ATOM | 968 | C | TRP | A | 141 | 2.962 | 48.383 | 0.743 | 1.00 | 209.93 | A | C |
| ATOM | 969 | O | TRP | A | 141 | 3.966 | 47.679 | 0.584 | 1.00 | 204.25 | A | O |
| ATOM | 970 | N | TRP | A | 142 | 1.718 | 47.914 | 0.623 | 1.00 | 219.90 | A | N |
| ATOM | 971 | CA | TRP | A | 142 | 1.423 | 46.646 | −0.051 | 1.00 | 209.42 | A | C |
| ATOM | 972 | CB | TRP | A | 142 | −0.056 | 46.581 | −0.439 | 1.00 | 233.63 | A | C |
| ATOM | 973 | CG | TRP | A | 142 | −0.922 | 45.733 | 0.469 | 1.00 | 236.48 | A | C |
| ATOM | 974 | CD1 | TRP | A | 142 | −0.947 | 45.731 | 1.869 | 1.00 | 181.85 | A | C |
| ATOM | 975 | NE1 | TRP | A | 142 | −1.868 | 44.828 | 2.341 | 1.00 | 181.39 | A | N |
| ATOM | 976 | CE2 | TRP | A | 142 | −2.491 | 44.200 | 1.312 | 1.00 | 218.44 | A | C |
| ATOM | 977 | CD2 | TRP | A | 142 | −1.935 | 44.740 | 0.066 | 1.00 | 257.17 | A | C |
| ATOM | 978 | CE3 | TRP | A | 142 | −2.416 | 44.255 | −1.164 | 1.00 | 268.57 | A | C |
| ATOM | 979 | CZ3 | TRP | A | 142 | −3.419 | 43.266 | −1.155 | 1.00 | 238.29 | A | C |
| ATOM | 980 | CH2 | TRP | A | 142 | −3.932 | 42.759 | 0.050 | 1.00 | 228.43 | A | C |
| ATOM | 981 | CZ2 | TRP | A | 142 | −3.477 | 43.219 | 1.306 | 1.00 | 217.51 | A | C |
| ATOM | 982 | C | TRP | A | 142 | 2.270 | 46.459 | −1.287 | 1.00 | 205.48 | A | C |
| ATOM | 983 | O | TRP | A | 142 | 2.699 | 45.346 | −1.603 | 1.00 | 195.01 | A | O |
| ATOM | 984 | N | ALA | A | 143 | 2.522 | 47.557 | −1.994 | 1.00 | 208.05 | A | N |
| ATOM | 985 | CA | ALA | A | 143 | 3.324 | 47.527 | −3.212 | 1.00 | 206.37 | A | C |
| ATOM | 986 | CB | ALA | A | 143 | 3.588 | 48.942 | −3.703 | 1.00 | 216.49 | A | C |
| ATOM | 987 | C | ALA | A | 143 | 4.641 | 46.777 | −3.023 | 1.00 | 217.21 | A | C |
| ATOM | 988 | O | ALA | A | 143 | 4.967 | 45.877 | −3.800 | 1.00 | 198.58 | A | O |
| ATOM | 989 | N | ILE | A | 144 | 5.374 | 47.149 | −1.972 | 1.00 | 248.48 | A | N |
| ATOM | 990 | CA | ILE | A | 144 | 6.744 | 46.665 | −1.737 | 1.00 | 209.00 | A | C |
| ATOM | 991 | CB | ILE | A | 144 | 7.552 | 47.642 | −0.839 | 1.00 | 219.83 | A | C |
| ATOM | 992 | CG1 | ILE | A | 144 | 6.989 | 47.665 | 0.593 | 1.00 | 213.55 | A | C |
| ATOM | 993 | CD1 | ILE | A | 144 | 7.493 | 48.798 | 1.465 | 1.00 | 186.23 | A | C |
| ATOM | 994 | CG2 | ILE | A | 144 | 7.596 | 49.040 | −1.462 | 1.00 | 159.19 | A | C |
| ATOM | 995 | C | ILE | A | 144 | 6.757 | 45.257 | −1.171 | 1.00 | 178.50 | A | C |
| ATOM | 996 | O | ILE | A | 144 | 7.763 | 44.552 | −1.263 | 1.00 | 214.09 | A | O |
| ATOM | 997 | N | ILE | A | 145 | 5.634 | 44.852 | −0.593 | 1.00 | 195.26 | A | N |
| ATOM | 998 | CA | ILE | A | 145 | 5.444 | 43.475 | −0.163 | 1.00 | 181.20 | A | C |
| ATOM | 999 | CB | ILE | A | 145 | 4.112 | 43.311 | 0.582 | 1.00 | 166.45 | A | C |
| ATOM | 1000 | CG1 | ILE | A | 145 | 4.236 | 43.861 | 2.007 | 1.00 | 201.76 | A | C |
| ATOM | 1001 | CD1 | ILE | A | 145 | 2.899 | 44.233 | 2.620 | 1.00 | 219.00 | A | C |
| ATOM | 1002 | CG2 | ILE | A | 145 | 3.684 | 41.861 | 0.593 | 1.00 | 172.44 | A | C |
| ATOM | 1003 | C | ILE | A | 145 | 5.478 | 42.527 | −1.355 | 1.00 | 179.13 | A | C |
| ATOM | 1004 | O | ILE | A | 145 | 6.217 | 41.539 | −1.334 | 1.00 | 165.71 | A | O |
| ATOM | 1005 | N | TRP | A | 146 | 4.676 | 42.827 | −2.383 | 1.00 | 198.70 | A | N |
| ATOM | 1006 | CA | TRP | A | 146 | 4.589 | 41.996 | −3.594 | 1.00 | 188.01 | A | C |
| ATOM | 1007 | CB | TRP | A | 146 | 3.663 | 42.639 | −4.605 | 1.00 | 200.84 | A | C |
| ATOM | 1008 | CG | TRP | A | 146 | 2.215 | 42.322 | −4.363 | 1.00 | 223.12 | A | C |
| ATOM | 1009 | CD1 | TRP | A | 146 | 1.216 | 43.185 | −3.925 | 1.00 | 219.21 | A | C |
| ATOM | 1010 | NE1 | TRP | A | 146 | 0.010 | 42.531 | −3.826 | 1.00 | 234.83 | A | N |
| ATOM | 1011 | CE2 | TRP | A | 146 | 0.143 | 41.233 | −4.177 | 1.00 | 253.16 | A | C |
| ATOM | 1012 | CD2 | TRP | A | 146 | 1.552 | 41.022 | −4.532 | 1.00 | 251.03 | A | C |
| ATOM | 1013 | CE3 | TRP | A | 146 | 1.967 | 39.751 | −4.935 | 1.00 | 240.01 | A | C |
| ATOM | 1014 | CZ3 | TRP | A | 146 | 1.020 | 38.723 | −4.976 | 1.00 | 215.20 | A | C |
| ATOM | 1015 | CH2 | TRP | A | 146 | −0.322 | 38.942 | −4.640 | 1.00 | 194.13 | A | C |
| ATOM | 1016 | CZ2 | TRP | A | 146 | −0.787 | 40.202 | −4.228 | 1.00 | 226.47 | A | C |
| ATOM | 1017 | C | TRP | A | 146 | 5.921 | 41.755 | −4.244 | 1.00 | 172.50 | A | C |
| ATOM | 1018 | O | TRP | A | 146 | 6.194 | 40.666 | −4.754 | 1.00 | 154.52 | A | O |
| ATOM | 1019 | N | LEU | A | 147 | 6.751 | 42.789 | −4.243 | 1.00 | 213.49 | A | N |
| ATOM | 1020 | CA | LEU | A | 147 | 8.094 | 42.697 | −4.774 | 1.00 | 202.69 | A | C |
| ATOM | 1021 | CB | LEU | A | 147 | 8.647 | 44.087 | −5.022 | 1.00 | 202.67 | A | C |
| ATOM | 1022 | CG | LEU | A | 147 | 7.862 | 44.924 | −6.026 | 1.00 | 184.58 | A | C |
| ATOM | 1023 | CD1 | LEU | A | 147 | 8.469 | 46.314 | −6.073 | 1.00 | 217.03 | A | C |
| ATOM | 1024 | CD2 | LEU | A | 147 | 7.827 | 44.271 | −7.405 | 1.00 | 184.22 | A | C |
| ATOM | 1025 | C | LEU | A | 147 | 8.986 | 41.933 | −3.820 | 1.00 | 207.94 | A | C |
| ATOM | 1026 | O | LEU | A | 147 | 9.908 | 41.256 | −4.268 | 1.00 | 207.72 | A | O |
| ATOM | 1027 | N | ALA | A | 148 | 8.698 | 42.023 | −2.518 | 1.00 | 207.27 | A | N |
| ATOM | 1028 | CA | ALA | A | 148 | 9.431 | 41.262 | −1.489 | 1.00 | 201.43 | A | C |
| ATOM | 1029 | CB | ALA | A | 148 | 9.116 | 41.786 | −0.090 | 1.00 | 196.62 | A | C |
| ATOM | 1030 | C | ALA | A | 148 | 9.142 | 39.756 | −1.584 | 1.00 | 201.33 | A | C |
| ATOM | 1031 | O | ALA | A | 148 | 10.056 | 38.917 | −1.506 | 1.00 | 191.80 | A | O |
| ATOM | 1032 | N | TRP | A | 149 | 7.863 | 39.418 | −1.751 | 1.00 | 182.10 | A | N |
| ATOM | 1033 | CA | TRP | A | 149 | 7.435 | 38.036 | −1.947 | 1.00 | 167.07 | A | C |
| ATOM | 1034 | CB | TRP | A | 149 | 5.927 | 37.905 | −1.864 | 1.00 | 155.92 | A | C |
| ATOM | 1035 | CG | TRP | A | 149 | 5.394 | 38.044 | −0.463 | 1.00 | 171.82 | A | C |
| ATOM | 1036 | CD1 | TRP | A | 149 | 6.108 | 37.985 | 0.733 | 1.00 | 191.30 | A | C |
| ATOM | 1037 | NE1 | TRP | A | 149 | 5.278 | 38.134 | 1.815 | 1.00 | 195.77 | A | N |
| ATOM | 1038 | CE2 | TRP | A | 149 | 4.000 | 38.291 | 1.411 | 1.00 | 198.38 | A | C |
| ATOM | 1039 | CD2 | TRP | A | 149 | 4.001 | 38.238 | −0.063 | 1.00 | 193.06 | A | C |
| ATOM | 1040 | CE3 | TRP | A | 149 | 2.802 | 38.375 | −0.746 | 1.00 | 175.12 | A | C |
| ATOM | 1041 | CZ3 | TRP | A | 149 | 1.634 | 38.559 | 0.000 | 1.00 | 200.12 | A | C |
| ATOM | 1042 | CH2 | TRP | A | 149 | 1.649 | 38.608 | 1.413 | 1.00 | 219.67 | A | C |
| ATOM | 1043 | CZ2 | TRP | A | 149 | 2.834 | 38.475 | 2.143 | 1.00 | 176.46 | A | C |
| ATOM | 1044 | C | TRP | A | 149 | 7.914 | 37.529 | −3.248 | 1.00 | 166.45 | A | C |
| ATOM | 1045 | O | TRP | A | 149 | 8.404 | 36.419 | −3.312 | 1.00 | 194.49 | A | O |
| ATOM | 1046 | N | GLY | A | 150 | 7.783 | 38.331 | −4.299 | 1.00 | 180.00 | A | N |
| ATOM | 1047 | CA | GLY | A | 150 | 8.371 | 38.000 | −5.597 | 1.00 | 185.95 | A | C |
| ATOM | 1048 | C | GLY | A | 150 | 9.843 | 37.664 | −5.460 | 1.00 | 202.56 | A | C |
| ATOM | 1049 | O | GLY | A | 150 | 10.367 | 36.812 | −6.196 | 1.00 | 199.47 | A | O |
| ATOM | 1050 | N | VAL | A | 151 | 10.495 | 38.314 | −4.493 | 1.00 | 204.53 | A | N |
| ATOM | 1051 | CA | VAL | A | 151 | 11.925 | 38.126 | −4.260 | 1.00 | 202.90 | A | C |
| ATOM | 1052 | CB | VAL | A | 151 | 12.487 | 39.213 | −3.316 | 1.00 | 202.41 | A | C |
| ATOM | 1053 | CG1 | VAL | A | 151 | 13.576 | 38.653 | −2.418 | 1.00 | 212.19 | A | C |
| ATOM | 1054 | CG2 | VAL | A | 151 | 13.012 | 40.389 | −4.128 | 1.00 | 225.44 | A | C |
| ATOM | 1055 | C | VAL | A | 151 | 12.232 | 36.730 | −3.741 | 1.00 | 197.13 | A | C |
| ATOM | 1056 | O | VAL | A | 151 | 13.009 | 35.990 | −4.354 | 1.00 | 208.08 | A | O |
| ATOM | 1057 | N | LEU | A | 152 | 11.623 | 36.377 | −2.609 | 1.00 | 193.85 | A | N |
| ATOM | 1058 | CA | LEU | A | 152 | 11.732 | 35.020 | −2.087 | 1.00 | 190.02 | A | C |
| ATOM | 1059 | CB | LEU | A | 152 | 10.742 | 34.787 | −0.956 | 1.00 | 179.06 | A | C |
| ATOM | 1060 | CG | LEU | A | 152 | 11.330 | 34.629 | 0.445 | 1.00 | 157.47 | A | C |
| ATOM | 1061 | CD1 | LEU | A | 152 | 10.203 | 34.469 | 1.444 | 1.00 | 166.04 | A | C |
| ATOM | 1062 | CD2 | LEU | A | 152 | 12.305 | 33.470 | 0.525 | 1.00 | 152.45 | A | C |
| ATOM | 1063 | C | LEU | A | 152 | 11.539 | 33.989 | −3.183 | 1.00 | 176.89 | A | C |
| ATOM | 1064 | O | LEU | A | 152 | 12.432 | 33.199 | −3.450 | 1.00 | 193.92 | A | O |
| ATOM | 1065 | N | TRP | A | 153 | 10.383 | 34.026 | −3.837 | 1.00 | 181.15 | A | N |
| ATOM | 1066 | CA | TRP | A | 153 | 10.017 | 33.022 | −4.844 | 1.00 | 192.69 | A | C |
| ATOM | 1067 | CB | TRP | A | 153 | 8.649 | 33.349 | −5.442 | 1.00 | 196.33 | A | C |
| ATOM | 1068 | CG | TRP | A | 153 | 7.515 | 32.775 | −4.649 | 1.00 | 192.38 | A | C |
| ATOM | 1069 | CD1 | TRP | A | 153 | 6.548 | 31.881 | −5.089 | 1.00 | 182.76 | A | C |
| ATOM | 1070 | NE1 | TRP | A | 153 | 5.696 | 31.546 | −4.072 | 1.00 | 165.14 | A | N |
| ATOM | 1071 | CE2 | TRP | A | 153 | 6.052 | 32.147 | −2.922 | 1.00 | 155.40 | A | C |
| ATOM | 1072 | CD2 | TRP | A | 153 | 7.226 | 32.972 | −3.218 | 1.00 | 174.16 | A | C |
| ATOM | 1073 | CE3 | TRP | A | 153 | 7.811 | 33.704 | −2.191 | 1.00 | 179.63 | A | C |
| ATOM | 1074 | CZ3 | TRP | A | 153 | 7.235 | 33.647 | −0.916 | 1.00 | 182.29 | A | C |
| ATOM | 1075 | CH2 | TRP | A | 153 | 6.096 | 32.864 | −0.659 | 1.00 | 171.17 | A | C |
| ATOM | 1076 | CZ2 | TRP | A | 153 | 5.487 | 32.098 | −1.656 | 1.00 | 160.04 | A | C |
| ATOM | 1077 | C | TRP | A | 153 | 11.046 | 32.897 | −5.922 | 1.00 | 160.13 | A | C |
| ATOM | 1078 | O | TRP | A | 153 | 11.367 | 31.793 | −6.366 | 1.00 | 183.39 | A | O |
| ATOM | 1079 | N | LEU | A | 154 | 11.594 | 34.039 | −6.335 | 1.00 | 196.79 | A | N |
| ATOM | 1080 | CA | LEU | A | 154 | 12.596 | 34.088 | −7.380 | 1.00 | 205.40 | A | C |
| ATOM | 1081 | CB | LEU | A | 154 | 12.871 | 35.542 | −7.742 | 1.00 | 193.98 | A | C |
| ATOM | 1082 | CG | LEU | A | 154 | 13.593 | 35.765 | −9.057 | 1.00 | 242.51 | A | C |
| ATOM | 1083 | CD1 | LEU | A | 154 | 13.069 | 34.834 | −10.135 | 1.00 | 215.01 | A | C |
| ATOM | 1084 | CD2 | LEU | A | 154 | 13.387 | 37.211 | −9.474 | 1.00 | 294.65 | A | C |
| ATOM | 1085 | C | LEU | A | 154 | 13.876 | 33.337 | −6.984 | 1.00 | 209.63 | A | C |
| ATOM | 1086 | O | LEU | A | 154 | 14.530 | 32.720 | −7.827 | 1.00 | 199.48 | A | O |
| ATOM | 1087 | N | THR | A | 155 | 14.215 | 33.377 | −5.697 | 1.00 | 214.33 | A | N |
| ATOM | 1088 | CA | THR | A | 155 | 15.431 | 32.741 | −5.199 | 1.00 | 189.27 | A | C |
| ATOM | 1089 | CB | THR | A | 155 | 15.580 | 32.880 | −3.674 | 1.00 | 187.11 | A | C |
| ATOM | 1090 | OG1 | THR | A | 155 | 14.551 | 32.123 | −3.009 | 1.00 | 175.43 | A | O |
| ATOM | 1091 | CG2 | THR | A | 155 | 15.526 | 34.354 | −3.260 | 1.00 | 177.05 | A | C |
| ATOM | 1092 | C | THR | A | 155 | 15.435 | 31.270 | −5.566 | 1.00 | 191.89 | A | C |
| ATOM | 1093 | O | THR | A | 155 | 16.422 | 30.754 | −6.085 | 1.00 | 203.18 | A | O |
| ATOM | 1094 | N | ALA | A | 156 | 14.314 | 30.598 | −5.305 | 1.00 | 171.95 | A | N |
| ATOM | 1095 | CA | ALA | A | 156 | 14.131 | 29.239 | −5.754 | 1.00 | 160.36 | A | C |
| ATOM | 1096 | CB | ALA | A | 156 | 12.714 | 28.791 | −5.511 | 1.00 | 160.92 | A | C |
| ATOM | 1097 | C | ALA | A | 156 | 14.482 | 29.113 | −7.234 | 1.00 | 192.92 | A | C |
| ATOM | 1098 | O | ALA | A | 156 | 15.025 | 28.092 | −7.640 | 1.00 | 204.38 | A | O |
| ATOM | 1099 | N | PHE | A | 157 | 14.208 | 30.145 | −8.028 | 1.00 | 188.96 | A | N |
| ATOM | 1100 | CA | PHE | A | 157 | 14.446 | 30.060 | −9.463 | 1.00 | 194.56 | A | C |
| ATOM | 1101 | CB | PHE | A | 157 | 13.749 | 31.175 | −10.228 | 1.00 | 183.75 | A | C |
| ATOM | 1102 | CG | PHE | A | 157 | 14.266 | 31.343 | −11.610 | 1.00 | 194.29 | A | C |
| ATOM | 1103 | CD1 | PHE | A | 157 | 13.919 | 30.463 | −12.617 | 1.00 | 218.94 | A | C |
| ATOM | 1104 | CE1 | PHE | A | 157 | 14.417 | 30.611 | −13.900 | 1.00 | 209.39 | A | C |
| ATOM | 1105 | CZ | PHE | A | 157 | 15.288 | 31.661 | −14.195 | 1.00 | 242.38 | A | C |
| ATOM | 1106 | CE2 | PHE | A | 157 | 15.658 | 32.542 | −13.191 | 1.00 | 260.31 | A | C |
| ATOM | 1107 | CD2 | PHE | A | 157 | 15.147 | 32.379 | −11.907 | 1.00 | 242.49 | A | C |
| ATOM | 1108 | C | PHE | A | 157 | 15.915 | 30.041 | −9.827 | 1.00 | 196.20 | A | C |
| ATOM | 1109 | O | PHE | A | 157 | 16.391 | 29.069 | −10.384 | 1.00 | 217.71 | A | O |
| ATOM | 1110 | N | ILE | A | 158 | 16.620 | 31.120 | −9.521 | 1.00 | 236.91 | A | N |
| ATOM | 1111 | CA | ILE | A | 158 | 18.032 | 31.251 | −9.881 | 1.00 | 240.24 | A | C |
| ATOM | 1112 | CB | ILE | A | 158 | 18.675 | 32.537 | −9.307 | 1.00 | 234.22 | A | C |
| ATOM | 1113 | CG1 | ILE | A | 158 | 18.288 | 32.745 | −7.840 | 1.00 | 206.47 | A | C |
| ATOM | 1114 | CD1 | ILE | A | 158 | 19.277 | 33.535 | −7.008 | 1.00 | 196.99 | A | C |
| ATOM | 1115 | CG2 | ILE | A | 158 | 18.271 | 33.740 | −10.146 | 1.00 | 235.21 | A | C |
| ATOM | 1116 | C | ILE | A | 158 | 18.814 | 30.060 | −9.385 | 1.00 | 219.24 | A | C |
| ATOM | 1117 | O | ILE | A | 158 | 19.514 | 29.396 | −10.152 | 1.00 | 217.05 | A | O |
| ATOM | 1118 | N | GLU | A | 159 | 18.656 | 29.785 | −8.095 | 1.00 | 231.04 | A | N |
| ATOM | 1119 | CA | GLU | A | 159 | 19.427 | 28.763 | −7.418 | 1.00 | 238.35 | A | C |
| ATOM | 1120 | CB | GLU | A | 159 | 19.083 | 28.725 | −5.932 | 1.00 | 241.49 | A | C |
| ATOM | 1121 | CG | GLU | A | 159 | 19.962 | 27.781 | −5.133 | 1.00 | 201.81 | A | C |
| ATOM | 1122 | CD | GLU | A | 159 | 20.056 | 28.175 | −3.670 | 1.00 | 241.93 | A | C |
| ATOM | 1123 | OE1 | GLU | A | 159 | 19.026 | 28.518 | −3.040 | 1.00 | 263.36 | A | O |
| ATOM | 1124 | OE2 | GLU | A | 159 | 21.182 | 28.147 | −3.144 | 1.00 | 232.78 | A | O |
| ATOM | 1125 | C | GLU | A | 159 | 19.248 | 27.397 | −8.026 | 1.00 | 219.69 | A | C |
| ATOM | 1126 | O | GLU | A | 159 | 20.212 | 26.648 | −8.174 | 1.00 | 194.88 | A | O |
| ATOM | 1127 | N | ASN | A | 160 | 18.014 | 27.091 | −8.391 | 1.00 | 204.33 | A | N |
| ATOM | 1128 | CA | ASN | A | 160 | 17.663 | 25.740 | −8.767 | 1.00 | 188.07 | A | C |
| ATOM | 1129 | CB | ASN | A | 160 | 16.297 | 25.352 | −8.186 | 1.00 | 193.61 | A | C |
| ATOM | 1130 | CG | ASN | A | 160 | 16.383 | 24.879 | −6.744 | 1.00 | 181.59 | A | C |
| ATOM | 1131 | OD1 | ASN | A | 160 | 17.271 | 24.102 | −6.392 | 1.00 | 202.16 | A | O |
| ATOM | 1132 | ND2 | ASN | A | 160 | 15.445 | 25.331 | −5.904 | 1.00 | 155.53 | A | N |
| ATOM | 1133 | C | ASN | A | 160 | 17.694 | 25.457 | −10.252 | 1.00 | 200.23 | A | C |
| ATOM | 1134 | O | ASN | A | 160 | 18.135 | 24.385 | −10.644 | 1.00 | 211.56 | A | O |
| ATOM | 1135 | N | ILE | A | 161 | 17.231 | 26.391 | −11.081 | 1.00 | 222.79 | A | N |
| ATOM | 1136 | CA | ILE | A | 161 | 16.913 | 26.021 | −12.464 | 1.00 | 227.06 | A | C |
| ATOM | 1137 | CB | ILE | A | 161 | 15.635 | 26.678 | −13.018 | 1.00 | 232.73 | A | C |
| ATOM | 1138 | CG1 | ILE | A | 161 | 15.228 | 25.959 | −14.304 | 1.00 | 236.91 | A | C |
| ATOM | 1139 | CD1 | ILE | A | 161 | 15.088 | 24.439 | −14.195 | 1.00 | 218.30 | A | C |
| ATOM | 1140 | CG2 | ILE | A | 161 | 15.851 | 28.156 | −13.263 | 1.00 | 223.08 | A | C |
| ATOM | 1141 | C | ILE | A | 161 | 18.039 | 26.066 | −13.488 | 1.00 | 263.08 | A | C |
| ATOM | 1142 | O | ILE | A | 161 | 18.209 | 25.124 | −14.264 | 1.00 | 293.14 | A | O |
| ATOM | 1143 | N | LEU | A | 162 | 18.772 | 27.171 | −13.524 | 1.00 | 241.15 | A | N |
| ATOM | 1144 | CA | LEU | A | 162 | 19.971 | 27.209 | −14.329 | 1.00 | 318.69 | A | C |
| ATOM | 1145 | CB | LEU | A | 162 | 19.658 | 27.191 | −15.834 | 1.00 | 364.85 | A | C |
| ATOM | 1146 | CG | LEU | A | 162 | 20.612 | 26.328 | −16.677 | 1.00 | 353.87 | A | C |
| ATOM | 1147 | CD1 | LEU | A | 162 | 20.737 | 24.910 | −16.106 | 1.00 | 211.60 | A | C |
| ATOM | 1148 | CD2 | LEU | A | 162 | 20.183 | 26.283 | −18.142 | 1.00 | 335.25 | A | C |
| ATOM | 1149 | C | LEU | A | 162 | 20.788 | 28.421 | −13.949 | 1.00 | 307.59 | A | C |
| ATOM | 1150 | O | LEU | A | 162 | 21.226 | 29.197 | −14.812 | 1.00 | 386.35 | A | O |
| ATOM | 1151 | N | LYS | A | 163 | 20.958 | 28.598 | −12.640 | 1.00 | 305.15 | A | N |
| ATOM | 1152 | CA | LYS | A | 163 | 21.963 | 29.528 | −12.139 | 1.00 | 300.54 | A | C |
| ATOM | 1153 | CB | LYS | A | 163 | 21.377 | 30.859 | −11.685 | 1.00 | 273.73 | A | C |
| ATOM | 1154 | CG | LYS | A | 163 | 22.449 | 31.677 | −10.989 | 1.00 | 258.67 | A | C |
| ATOM | 1155 | CD | LYS | A | 163 | 21.935 | 32.982 | −10.455 | 1.00 | 263.29 | A | C |
| ATOM | 1156 | CE | LYS | A | 163 | 23.076 | 34.014 | −10.251 | 1.00 | 332.17 | A | C |
| ATOM | 1157 | NZ | LYS | A | 163 | 23.011 | 35.427 | −10.869 | 1.00 | 398.09 | A | N |
| ATOM | 1158 | C | LYS | A | 163 | 22.853 | 28.994 | −11.024 | 1.00 | 285.22 | A | C |
| ATOM | 1159 | O | LYS | A | 163 | 23.996 | 29.434 | −10.907 | 1.00 | 310.48 | A | O |
| ATOM | 1160 | N | ILE | A | 164 | 22.325 | 28.115 | −10.179 | 1.00 | 240.53 | A | N |
| ATOM | 1161 | CA | ILE | A | 164 | 23.094 | 27.514 | −9.065 | 1.00 | 234.92 | A | C |
| ATOM | 1162 | CB | ILE | A | 164 | 23.859 | 26.232 | −9.515 | 1.00 | 242.82 | A | C |
| ATOM | 1163 | CG1 | ILE | A | 164 | 24.818 | 26.530 | −10.681 | 1.00 | 269.97 | A | C |
| ATOM | 1164 | CD1 | ILE | A | 164 | 25.908 | 25.494 | −10.901 | 1.00 | 306.72 | A | C |
| ATOM | 1165 | CG2 | ILE | A | 164 | 22.875 | 25.121 | −9.882 | 1.00 | 283.85 | A | C |
| ATOM | 1166 | C | ILE | A | 164 | 23.990 | 28.477 | −8.227 | 1.00 | 225.36 | A | C |
| ATOM | 1167 | O | ILE | A | 164 | 25.210 | 28.328 | −8.181 | 1.00 | 236.26 | A | O |
| ATOM | 1168 | N | PRO | A | 165 | 23.380 | 29.487 | −7.577 | 1.00 | 223.88 | A | N |
| ATOM | 1169 | CA | PRO | A | 165 | 24.041 | 30.390 | −6.648 | 1.00 | 215.86 | A | C |
| ATOM | 1170 | CB | PRO | A | 165 | 23.295 | 31.696 | −6.896 | 1.00 | 249.17 | A | C |
| ATOM | 1171 | CG | PRO | A | 165 | 21.901 | 31.217 | −7.083 | 1.00 | 242.75 | A | C |
| ATOM | 1172 | CD | PRO | A | 165 | 22.071 | 30.027 | −7.973 | 1.00 | 240.13 | A | C |
| ATOM | 1173 | C | PRO | A | 165 | 23.854 | 29.971 | −5.178 | 1.00 | 221.36 | A | C |
| ATOM | 1174 | O | PRO | A | 165 | 23.373 | 28.896 | −4.902 | 1.00 | 211.75 | A | O |
| ATOM | 1175 | N | LEU | A | 166 | 24.136 | 30.887 | −4.266 | 1.00 | 262.00 | A | N |
| ATOM | 1176 | CA | LEU | A | 166 | 24.304 | 30.623 | −2.837 | 1.00 | 265.07 | A | C |
| ATOM | 1177 | CB | LEU | A | 166 | 25.474 | 31.485 | −2.321 | 1.00 | 277.42 | A | C |
| ATOM | 1178 | CG | LEU | A | 166 | 26.483 | 32.170 | −3.254 | 1.00 | 268.61 | A | C |
| ATOM | 1179 | CD1 | LEU | A | 166 | 26.848 | 33.558 | −2.709 | 1.00 | 305.87 | A | C |
| ATOM | 1180 | CD2 | LEU | A | 166 | 27.674 | 31.241 | −3.426 | 1.00 | 281.86 | A | C |
| ATOM | 1181 | C | LEU | A | 166 | 23.055 | 30.987 | −2.018 | 1.00 | 225.67 | A | C |
| ATOM | 1182 | O | LEU | A | 166 | 22.253 | 31.858 | −2.428 | 1.00 | 229.94 | A | O |
| ATOM | 1183 | N | GLY | A | 167 | 22.937 | 30.382 | −0.838 | 1.00 | 209.59 | A | N |
| ATOM | 1184 | CA | GLY | A | 167 | 21.678 | 30.448 | −0.156 | 1.00 | 239.37 | A | C |
| ATOM | 1185 | C | GLY | A | 167 | 21.496 | 30.708 | 1.323 | 1.00 | 231.15 | A | C |
| ATOM | 1186 | O | GLY | A | 167 | 20.355 | 30.801 | 1.759 | 1.00 | 261.43 | A | O |
| ATOM | 1187 | N | LYS | A | 168 | 22.555 | 30.769 | 2.112 | 1.00 | 219.34 | A | N |
| ATOM | 1188 | CA | LYS | A | 168 | 22.404 | 30.814 | 3.595 | 1.00 | 228.99 | A | C |
| ATOM | 1189 | CB | LYS | A | 168 | 23.782 | 30.871 | 4.284 | 1.00 | 249.46 | A | C |
| ATOM | 1190 | CG | LYS | A | 168 | 23.866 | 31.520 | 5.670 | 1.00 | 263.58 | A | C |
| ATOM | 1191 | CD | LYS | A | 168 | 25.329 | 31.847 | 6.021 | 1.00 | 286.14 | A | C |
| ATOM | 1192 | CE | LYS | A | 168 | 25.526 | 33.199 | 6.711 | 1.00 | 293.99 | A | C |
| ATOM | 1193 | NZ | LYS | A | 168 | 26.939 | 33.654 | 6.531 | 1.00 | 390.13 | A | N |
| ATOM | 1194 | C | LYS | A | 168 | 21.397 | 31.838 | 4.193 | 1.00 | 208.27 | A | C |
| ATOM | 1195 | O | LYS | A | 168 | 20.937 | 31.636 | 5.314 | 1.00 | 223.71 | A | O |
| ATOM | 1196 | N | PHE | A | 169 | 21.066 | 32.911 | 3.467 | 1.00 | 213.54 | A | N |
| ATOM | 1197 | CA | PHE | A | 169 | 20.067 | 33.917 | 3.927 | 1.00 | 248.91 | A | C |
| ATOM | 1198 | CB | PHE | A | 169 | 20.579 | 35.397 | 3.770 | 1.00 | 308.25 | A | C |
| ATOM | 1199 | CG | PHE | A | 169 | 21.059 | 36.043 | 5.081 | 1.00 | 249.54 | A | C |
| ATOM | 1200 | CD1 | PHE | A | 169 | 22.249 | 35.607 | 5.685 | 1.00 | 303.40 | A | C |
| ATOM | 1201 | CE1 | PHE | A | 169 | 22.687 | 36.134 | 6.896 | 1.00 | 374.93 | A | C |
| ATOM | 1202 | CZ | PHE | A | 169 | 21.932 | 37.099 | 7.530 | 1.00 | 409.21 | A | C |
| ATOM | 1203 | CE2 | PHE | A | 169 | 20.766 | 37.576 | 6.947 | 1.00 | 333.45 | A | C |
| ATOM | 1204 | CD2 | PHE | A | 169 | 20.331 | 37.058 | 5.723 | 1.00 | 250.30 | A | C |
| ATOM | 1205 | C | PHE | A | 169 | 18.683 | 33.714 | 3.273 | 1.00 | 199.68 | A | C |
| ATOM | 1206 | O | PHE | A | 169 | 17.694 | 34.303 | 3.714 | 1.00 | 199.98 | A | O |
| ATOM | 1207 | N | THR | A | 170 | 18.629 | 32.895 | 2.212 | 1.00 | 218.61 | A | N |
| ATOM | 1208 | CA | THR | A | 170 | 17.358 | 32.553 | 1.537 | 1.00 | 225.48 | A | C |
| ATOM | 1209 | CB | THR | A | 170 | 17.539 | 31.513 | 0.411 | 1.00 | 189.02 | A | C |
| ATOM | 1210 | OG1 | THR | A | 170 | 18.567 | 31.945 | −0.480 | 1.00 | 217.60 | A | O |
| ATOM | 1211 | CG2 | THR | A | 170 | 16.233 | 31.301 | −0.364 | 1.00 | 157.09 | A | C |
| ATOM | 1212 | C | THR | A | 170 | 16.315 | 32.045 | 2.519 | 1.00 | 185.05 | A | C |
| ATOM | 1213 | O | THR | A | 170 | 15.138 | 32.376 | 2.390 | 1.00 | 194.96 | A | O |
| ATOM | 1214 | N | PRO | A | 171 | 16.738 | 31.228 | 3.494 | 1.00 | 181.15 | A | N |
| ATOM | 1215 | CA | PRO | A | 171 | 15.887 | 30.905 | 4.635 | 1.00 | 173.36 | A | C |
| ATOM | 1216 | CB | PRO | A | 171 | 16.717 | 29.872 | 5.414 | 1.00 | 149.91 | A | C |
| ATOM | 1217 | CG | PRO | A | 171 | 18.103 | 29.996 | 4.880 | 1.00 | 176.96 | A | C |
| ATOM | 1218 | CD | PRO | A | 171 | 17.942 | 30.388 | 3.456 | 1.00 | 187.73 | A | C |
| ATOM | 1219 | C | PRO | A | 171 | 15.567 | 32.145 | 5.489 | 1.00 | 197.86 | A | C |
| ATOM | 1220 | O | PRO | A | 171 | 14.416 | 32.354 | 5.879 | 1.00 | 213.37 | A | O |
| ATOM | 1221 | N | TRP | A | 172 | 16.570 | 32.971 | 5.754 | 1.00 | 188.09 | A | N |
| ATOM | 1222 | CA | TRP | A | 172 | 16.386 | 34.152 | 6.609 | 1.00 | 199.76 | A | C |
| ATOM | 1223 | CB | TRP | A | 172 | 17.725 | 34.816 | 6.933 | 1.00 | 193.63 | A | C |
| ATOM | 1224 | CG | TRP | A | 172 | 18.519 | 33.939 | 7.862 | 1.00 | 222.97 | A | C |
| ATOM | 1225 | CD1 | TRP | A | 172 | 19.650 | 33.186 | 7.562 | 1.00 | 220.49 | A | C |
| ATOM | 1226 | NE1 | TRP | A | 172 | 20.071 | 32.485 | 8.664 | 1.00 | 235.99 | A | N |
| ATOM | 1227 | CE2 | TRP | A | 172 | 19.259 | 32.720 | 9.730 | 1.00 | 242.14 | A | C |
| ATOM | 1228 | CD2 | TRP | A | 172 | 18.225 | 33.654 | 9.281 | 1.00 | 235.77 | A | C |
| ATOM | 1229 | CE3 | TRP | A | 172 | 17.246 | 34.077 | 10.193 | 1.00 | 201.35 | A | C |
| ATOM | 1230 | CZ3 | TRP | A | 172 | 17.292 | 33.587 | 11.505 | 1.00 | 179.67 | A | C |
| ATOM | 1231 | CH2 | TRP | A | 172 | 18.295 | 32.689 | 11.914 | 1.00 | 220.19 | A | C |
| ATOM | 1232 | CZ2 | TRP | A | 172 | 19.300 | 32.244 | 11.038 | 1.00 | 247.43 | A | C |
| ATOM | 1233 | C | TRP | A | 172 | 15.387 | 35.088 | 6.002 | 1.00 | 203.95 | A | C |
| ATOM | 1234 | O | TRP | A | 172 | 14.459 | 35.551 | 6.678 | 1.00 | 184.54 | A | O |
| ATOM | 1235 | N | LEU | A | 173 | 15.545 | 35.338 | 4.706 | 1.00 | 201.15 | A | N |
| ATOM | 1236 | CA | LEU | A | 173 | 14.584 | 36.097 | 3.930 | 1.00 | 184.45 | A | C |
| ATOM | 1237 | CB | LEU | A | 173 | 14.870 | 35.920 | 2.442 | 1.00 | 184.91 | A | C |
| ATOM | 1238 | CG | LEU | A | 173 | 13.968 | 36.674 | 1.477 | 1.00 | 180.47 | A | C |
| ATOM | 1239 | CD1 | LEU | A | 173 | 13.882 | 38.144 | 1.844 | 1.00 | 178.94 | A | C |
| ATOM | 1240 | CD2 | LEU | A | 173 | 14.465 | 36.507 | 0.058 | 1.00 | 196.04 | A | C |
| ATOM | 1241 | C | LEU | A | 173 | 13.178 | 35.630 | 4.252 | 1.00 | 186.41 | A | C |
| ATOM | 1242 | O | LEU | A | 173 | 12.292 | 36.434 | 4.512 | 1.00 | 185.18 | A | O |
| ATOM | 1243 | N | ALA | A | 174 | 12.992 | 34.315 | 4.253 | 1.00 | 180.24 | A | N |
| ATOM | 1244 | CA | ALA | A | 174 | 11.705 | 33.725 | 4.525 | 1.00 | 157.86 | A | C |
| ATOM | 1245 | CB | ALA | A | 174 | 11.722 | 32.271 | 4.111 | 1.00 | 108.00 | A | C |
| ATOM | 1246 | C | ALA | A | 174 | 11.305 | 33.856 | 5.992 | 1.00 | 192.16 | A | C |
| ATOM | 1247 | O | ALA | A | 174 | 10.113 | 33.924 | 6.309 | 1.00 | 199.09 | A | O |
| ATOM | 1248 | N | ILE | A | 175 | 12.293 | 33.890 | 6.884 | 1.00 | 179.45 | A | N |
| ATOM | 1249 | CA | ILE | A | 175 | 12.008 | 33.956 | 8.319 | 1.00 | 165.91 | A | C |
| ATOM | 1250 | CB | ILE | A | 175 | 13.197 | 33.512 | 9.177 | 1.00 | 163.96 | A | C |
| ATOM | 1251 | CG1 | ILE | A | 175 | 13.591 | 32.096 | 8.798 | 1.00 | 168.62 | A | C |
| ATOM | 1252 | CD1 | ILE | A | 175 | 15.058 | 31.804 | 8.963 | 1.00 | 199.14 | A | C |
| ATOM | 1253 | CG2 | ILE | A | 175 | 12.833 | 33.571 | 10.655 | 1.00 | 168.36 | A | C |
| ATOM | 1254 | C | ILE | A | 175 | 11.568 | 35.348 | 8.719 | 1.00 | 168.62 | A | C |
| ATOM | 1255 | O | ILE | A | 175 | 10.474 | 35.509 | 9.245 | 1.00 | 195.95 | A | O |
| ATOM | 1256 | N | ILE | A | 176 | 12.418 | 36.341 | 8.474 | 1.00 | 171.69 | A | N |
| ATOM | 1257 | CA | ILE | A | 176 | 12.017 | 37.738 | 8.626 | 1.00 | 188.32 | A | C |
| ATOM | 1258 | CB | ILE | A | 176 | 13.095 | 38.712 | 8.091 | 1.00 | 228.41 | A | C |
| ATOM | 1259 | CG1 | ILE | A | 176 | 13.356 | 38.433 | 6.606 | 1.00 | 196.43 | A | C |
| ATOM | 1260 | CD1 | ILE | A | 176 | 14.475 | 39.227 | 5.986 | 1.00 | 167.35 | A | C |
| ATOM | 1261 | CG2 | ILE | A | 176 | 14.373 | 38.657 | 8.937 | 1.00 | 214.56 | A | C |
| ATOM | 1262 | C | ILE | A | 176 | 10.684 | 38.025 | 7.915 | 1.00 | 197.51 | A | C |
| ATOM | 1263 | O | ILE | A | 176 | 9.768 | 38.611 | 8.509 | 1.00 | 188.28 | A | O |
| ATOM | 1264 | N | GLU | A | 177 | 10.582 | 37.592 | 6.654 | 1.00 | 190.96 | A | N |
| ATOM | 1265 | CA | GLU | A | 177 | 9.382 | 37.806 | 5.848 | 1.00 | 177.33 | A | C |
| ATOM | 1266 | CB | GLU | A | 177 | 9.570 | 37.304 | 4.420 | 1.00 | 176.35 | A | C |
| ATOM | 1267 | CG | GLU | A | 177 | 9.787 | 38.421 | 3.411 | 1.00 | 178.42 | A | C |
| ATOM | 1268 | CD | GLU | A | 177 | 9.696 | 37.914 | 1.992 | 1.00 | 164.65 | A | C |
| ATOM | 1269 | OE1 | GLU | A | 177 | 10.668 | 38.072 | 1.241 | 1.00 | 163.65 | A | O |
| ATOM | 1270 | OE2 | GLU | A | 177 | 8.657 | 37.336 | 1.630 | 1.00 | 180.79 | A | O |
| ATOM | 1271 | C | GLU | A | 177 | 8.162 | 37.157 | 6.477 | 1.00 | 202.58 | A | C |
| ATOM | 1272 | O | GLU | A | 177 | 7.029 | 37.580 | 6.231 | 1.00 | 194.98 | A | O |
| ATOM | 1273 | N | GLY | A | 178 | 8.408 | 36.125 | 7.282 | 1.00 | 187.34 | A | N |
| ATOM | 1274 | CA | GLY | A | 178 | 7.375 | 35.508 | 8.114 | 1.00 | 189.15 | A | C |
| ATOM | 1275 | C | GLY | A | 178 | 6.883 | 36.460 | 9.193 | 1.00 | 199.33 | A | C |
| ATOM | 1276 | O | GLY | A | 178 | 5.713 | 36.855 | 9.195 | 1.00 | 184.12 | A | O |
| ATOM | 1277 | N | ILE | A | 179 | 7.779 | 36.846 | 10.099 | 1.00 | 204.41 | A | N |
| ATOM | 1278 | CA | ILE | A | 179 | 7.434 | 37.789 | 11.173 | 1.00 | 202.23 | A | C |
| ATOM | 1279 | CB | ILE | A | 179 | 8.633 | 38.120 | 12.088 | 1.00 | 216.01 | A | C |
| ATOM | 1280 | CG1 | ILE | A | 179 | 9.194 | 36.854 | 12.742 | 1.00 | 192.16 | A | C |
| ATOM | 1281 | CD1 | ILE | A | 179 | 10.492 | 37.062 | 13.499 | 1.00 | 233.65 | A | C |
| ATOM | 1282 | CG2 | ILE | A | 179 | 8.194 | 39.105 | 13.163 | 1.00 | 204.36 | A | C |
| ATOM | 1283 | C | ILE | A | 179 | 6.882 | 39.103 | 10.629 | 1.00 | 184.30 | A | C |
| ATOM | 1284 | O | ILE | A | 179 | 5.859 | 39.619 | 11.101 | 1.00 | 185.33 | A | O |
| ATOM | 1285 | N | LEU | A | 180 | 7.571 | 39.636 | 9.630 | 1.00 | 190.76 | A | N |
| ATOM | 1286 | CA | LEU | A | 180 | 7.218 | 40.933 | 9.094 | 1.00 | 194.49 | A | C |
| ATOM | 1287 | CB | LEU | A | 180 | 8.415 | 41.580 | 8.375 | 1.00 | 200.71 | A | C |
| ATOM | 1288 | CG | LEU | A | 180 | 9.288 | 42.700 | 8.939 | 1.00 | 190.54 | A | C |
| ATOM | 1289 | CD1 | LEU | A | 180 | 10.067 | 42.162 | 10.132 | 1.00 | 239.74 | A | C |
| ATOM | 1290 | CD2 | LEU | A | 180 | 10.229 | 43.205 | 7.832 | 1.00 | 164.21 | A | C |
| ATOM | 1291 | C | LEU | A | 180 | 6.047 | 40.891 | 8.130 | 1.00 | 190.86 | A | C |
| ATOM | 1292 | O | LEU | A | 180 | 4.981 | 41.437 | 8.416 | 1.00 | 183.09 | A | O |
| ATOM | 1293 | N | THR | A | 181 | 6.256 | 40.231 | 6.992 | 1.00 | 197.29 | A | N |
| ATOM | 1294 | CA | THR | A | 181 | 5.464 | 40.493 | 5.789 | 1.00 | 202.73 | A | C |
| ATOM | 1295 | CB | THR | A | 181 | 6.184 | 40.027 | 4.506 | 1.00 | 228.49 | A | C |
| ATOM | 1296 | OG1 | THR | A | 181 | 7.569 | 40.382 | 4.572 | 1.00 | 252.59 | A | O |
| ATOM | 1297 | CG2 | THR | A | 181 | 5.576 | 40.693 | 3.292 | 1.00 | 199.13 | A | C |
| ATOM | 1298 | C | THR | A | 181 | 4.090 | 39.876 | 5.846 | 1.00 | 176.52 | A | C |
| ATOM | 1299 | O | THR | A | 181 | 3.109 | 40.482 | 5.417 | 1.00 | 198.39 | A | O |
| ATOM | 1300 | N | ALA | A | 182 | 4.024 | 38.663 | 6.369 | 1.00 | 174.15 | A | N |
| ATOM | 1301 | CA | ALA | A | 182 | 2.761 | 37.992 | 6.480 | 1.00 | 175.63 | A | C |
| ATOM | 1302 | CB | ALA | A | 182 | 2.933 | 36.525 | 6.141 | 1.00 | 202.12 | A | C |
| ATOM | 1303 | C | ALA | A | 182 | 2.153 | 38.143 | 7.862 | 1.00 | 182.74 | A | C |
| ATOM | 1304 | O | ALA | A | 182 | 0.950 | 38.356 | 7.984 | 1.00 | 184.10 | A | O |
| ATOM | 1305 | N | TRP | A | 183 | 2.983 | 38.034 | 8.898 | 1.00 | 190.88 | A | N |
| ATOM | 1306 | CA | TRP | A | 183 | 2.499 | 37.798 | 10.257 | 1.00 | 173.65 | A | C |
| ATOM | 1307 | CB | TRP | A | 183 | 3.654 | 37.412 | 11.163 | 1.00 | 186.43 | A | C |
| ATOM | 1308 | CG | TRP | A | 183 | 3.249 | 36.995 | 12.560 | 1.00 | 175.85 | A | C |
| ATOM | 1309 | CD1 | TRP | A | 183 | 2.011 | 37.163 | 13.192 | 1.00 | 153.05 | A | C |
| ATOM | 1310 | NE1 | TRP | A | 183 | 2.040 | 36.657 | 14.462 | 1.00 | 155.91 | A | N |
| ATOM | 1311 | CE2 | TRP | A | 183 | 3.271 | 36.161 | 14.753 | 1.00 | 155.25 | A | C |
| ATOM | 1312 | CD2 | TRP | A | 183 | 4.104 | 36.347 | 13.563 | 1.00 | 183.13 | A | C |
| ATOM | 1313 | CE3 | TRP | A | 183 | 5.431 | 35.907 | 13.588 | 1.00 | 196.68 | A | C |
| ATOM | 1314 | CZ3 | TRP | A | 183 | 5.920 | 35.297 | 14.751 | 1.00 | 175.52 | A | C |
| ATOM | 1315 | CH2 | TRP | A | 183 | 5.105 | 35.125 | 15.880 | 1.00 | 186.31 | A | C |
| ATOM | 1316 | CZ2 | TRP | A | 183 | 3.767 | 35.559 | 15.904 | 1.00 | 139.35 | A | C |
| ATOM | 1317 | C | TRP | A | 183 | 1.782 | 38.965 | 10.847 | 1.00 | 170.69 | A | C |
| ATOM | 1318 | O | TRP | A | 183 | 0.613 | 38.876 | 11.193 | 1.00 | 185.79 | A | O |
| ATOM | 1319 | N | ILE | A | 184 | 2.500 | 40.069 | 11.000 | 1.00 | 177.37 | A | N |
| ATOM | 1320 | CA | ILE | A | 184 | 1.950 | 41.272 | 11.607 | 1.00 | 182.59 | A | C |
| ATOM | 1321 | CB | ILE | A | 184 | 3.070 | 42.284 | 11.893 | 1.00 | 170.94 | A | C |
| ATOM | 1322 | CG1 | ILE | A | 184 | 3.932 | 41.741 | 13.034 | 1.00 | 190.10 | A | C |
| ATOM | 1323 | CD1 | ILE | A | 184 | 5.372 | 42.159 | 12.947 | 1.00 | 192.55 | A | C |
| ATOM | 1324 | CG2 | ILE | A | 184 | 2.504 | 43.641 | 12.267 | 1.00 | 185.52 | A | C |
| ATOM | 1325 | C | ILE | A | 184 | 0.788 | 41.855 | 10.792 | 1.00 | 198.96 | A | C |
| ATOM | 1326 | O | ILE | A | 184 | −0.266 | 42.127 | 11.370 | 1.00 | 172.14 | A | O |
| ATOM | 1327 | N | PRO | A | 185 | 0.963 | 42.024 | 9.458 | 1.00 | 190.94 | A | N |
| ATOM | 1328 | CA | PRO | A | 185 | −0.087 | 42.546 | 8.564 | 1.00 | 180.52 | A | C |
| ATOM | 1329 | CB | PRO | A | 185 | 0.483 | 42.286 | 7.161 | 1.00 | 168.11 | A | C |
| ATOM | 1330 | CG | PRO | A | 185 | 1.943 | 42.449 | 7.376 | 1.00 | 159.83 | A | C |
| ATOM | 1331 | CD | PRO | A | 185 | 2.196 | 41.756 | 8.698 | 1.00 | 190.93 | A | C |
| ATOM | 1332 | C | PRO | A | 185 | −1.402 | 41.808 | 8.718 | 1.00 | 184.75 | A | C |
| ATOM | 1333 | O | PRO | A | 185 | −2.461 | 42.393 | 8.515 | 1.00 | 181.18 | A | O |
| ATOM | 1334 | N | ALA | A | 186 | −1.324 | 40.531 | 9.074 | 1.00 | 189.26 | A | N |
| ATOM | 1335 | CA | ALA | A | 186 | −2.510 | 39.744 | 9.349 | 1.00 | 187.75 | A | C |
| ATOM | 1336 | CB | ALA | A | 186 | −2.206 | 38.262 | 9.222 | 1.00 | 174.21 | A | C |
| ATOM | 1337 | C | ALA | A | 186 | −3.064 | 40.073 | 10.733 | 1.00 | 208.98 | A | C |
| ATOM | 1338 | O | ALA | A | 186 | −4.242 | 40.417 | 10.872 | 1.00 | 195.29 | A | O |
| ATOM | 1339 | N | TRP | A | 187 | −2.203 | 39.979 | 11.747 | 1.00 | 191.88 | A | N |
| ATOM | 1340 | CA | TRP | A | 187 | −2.575 | 40.296 | 13.110 | 1.00 | 186.77 | A | C |
| ATOM | 1341 | CB | TRP | A | 187 | −1.454 | 39.921 | 14.074 | 1.00 | 190.34 | A | C |
| ATOM | 1342 | CG | TRP | A | 187 | −1.845 | 40.176 | 15.494 | 1.00 | 200.15 | A | C |
| ATOM | 1343 | CD1 | TRP | A | 187 | −1.562 | 41.307 | 16.250 | 1.00 | 235.00 | A | C |
| ATOM | 1344 | NE1 | TRP | A | 187 | −2.110 | 41.213 | 17.501 | 1.00 | 254.87 | A | N |
| ATOM | 1345 | CE2 | TRP | A | 187 | −2.785 | 40.057 | 17.640 | 1.00 | 233.10 | A | C |
| ATOM | 1346 | CD2 | TRP | A | 187 | −2.660 | 39.330 | 16.373 | 1.00 | 198.92 | A | C |
| ATOM | 1347 | CE3 | TRP | A | 187 | −3.262 | 38.108 | 16.251 | 1.00 | 219.28 | A | C |
| ATOM | 1348 | CZ3 | TRP | A | 187 | −3.976 | 37.602 | 17.345 | 1.00 | 252.82 | A | C |
| ATOM | 1349 | CH2 | TRP | A | 187 | −4.079 | 38.303 | 18.549 | 1.00 | 202.58 | A | C |
| ATOM | 1350 | CZ2 | TRP | A | 187 | −3.493 | 39.545 | 18.717 | 1.00 | 189.06 | A | C |
| ATOM | 1351 | C | TRP | A | 187 | −2.960 | 41.748 | 13.188 | 1.00 | 187.43 | A | C |
| ATOM | 1352 | O | TRP | A | 187 | −3.709 | 42.159 | 14.075 | 1.00 | 184.49 | A | O |
| ATOM | 1353 | N | LEU | A | 188 | −2.480 | 42.520 | 12.216 | 1.00 | 188.34 | A | N |
| ATOM | 1354 | CA | LEU | A | 188 | −2.804 | 43.941 | 12.068 | 1.00 | 190.75 | A | C |
| ATOM | 1355 | CB | LEU | A | 188 | −1.545 | 44.705 | 11.663 | 1.00 | 182.74 | A | C |
| ATOM | 1356 | CG | LEU | A | 188 | −1.567 | 46.161 | 11.235 | 1.00 | 169.14 | A | C |
| ATOM | 1357 | CD1 | LEU | A | 188 | −0.230 | 46.747 | 11.604 | 1.00 | 161.72 | A | C |
| ATOM | 1358 | CD2 | LEU | A | 188 | −1.799 | 46.295 | 9.743 | 1.00 | 174.65 | A | C |
| ATOM | 1359 | C | LEU | A | 188 | −3.919 | 44.157 | 11.040 | 1.00 | 178.99 | A | C |
| ATOM | 1360 | O | LEU | A | 188 | −4.429 | 45.264 | 10.870 | 1.00 | 172.77 | A | O |
| ATOM | 1361 | N | LEU | A | 189 | −4.286 | 43.092 | 10.341 | 1.00 | 187.54 | A | N |
| ATOM | 1362 | CA | LEU | A | 189 | −5.447 | 43.132 | 9.469 | 1.00 | 189.94 | A | C |
| ATOM | 1363 | CB | LEU | A | 189 | −5.327 | 42.069 | 8.386 | 1.00 | 169.93 | A | C |
| ATOM | 1364 | CG | LEU | A | 189 | −5.771 | 42.489 | 6.993 | 1.00 | 186.19 | A | C |
| ATOM | 1365 | CD1 | LEU | A | 189 | −5.043 | 43.745 | 6.530 | 1.00 | 164.76 | A | C |
| ATOM | 1366 | CD2 | LEU | A | 189 | −5.518 | 41.342 | 6.031 | 1.00 | 184.15 | A | C |
| ATOM | 1367 | C | LEU | A | 189 | −6.716 | 42.908 | 10.285 | 1.00 | 191.28 | A | C |
| ATOM | 1368 | O | LEU | A | 189 | −7.815 | 43.251 | 9.853 | 1.00 | 188.01 | A | O |
| ATOM | 1369 | N | PHE | A | 190 | −6.556 | 42.317 | 11.468 | 1.00 | 181.47 | A | N |
| ATOM | 1370 | CA | PHE | A | 190 | −7.664 | 42.165 | 12.406 | 1.00 | 176.72 | A | C |
| ATOM | 1371 | CB | PHE | A | 190 | −7.318 | 41.184 | 13.540 | 1.00 | 168.60 | A | C |
| ATOM | 1372 | CG | PHE | A | 190 | −7.151 | 39.760 | 13.085 | 1.00 | 197.71 | A | C |
| ATOM | 1373 | CD1 | PHE | A | 190 | −8.113 | 39.158 | 12.281 | 1.00 | 209.25 | A | C |
| ATOM | 1374 | CE1 | PHE | A | 190 | −7.959 | 37.854 | 11.861 | 1.00 | 193.09 | A | C |
| ATOM | 1375 | CZ | PHE | A | 190 | −6.837 | 37.132 | 12.244 | 1.00 | 187.01 | A | C |
| ATOM | 1376 | CE2 | PHE | A | 190 | −5.875 | 37.714 | 13.048 | 1.00 | 165.54 | A | C |
| ATOM | 1377 | CD2 | PHE | A | 190 | −6.039 | 39.020 | 13.465 | 1.00 | 184.76 | A | C |
| ATOM | 1378 | C | PHE | A | 190 | −8.042 | 43.498 | 13.001 | 1.00 | 200.33 | A | C |
| ATOM | 1379 | O | PHE | A | 190 | −9.201 | 43.704 | 13.354 | 1.00 | 210.93 | A | O |
| ATOM | 1380 | N | ILE | A | 191 | −7.066 | 44.402 | 13.078 | 1.00 | 203.10 | A | N |
| ATOM | 1381 | CA | ILE | A | 191 | −7.169 | 45.617 | 13.884 | 1.00 | 190.75 | A | C |
| ATOM | 1382 | CB | ILE | A | 191 | −5.953 | 45.742 | 14.826 | 1.00 | 221.34 | A | C |
| ATOM | 1383 | CG1 | ILE | A | 191 | −6.050 | 44.676 | 15.934 | 1.00 | 249.86 | A | C |
| ATOM | 1384 | CD1 | ILE | A | 191 | −4.871 | 44.613 | 16.887 | 1.00 | 272.20 | A | C |
| ATOM | 1385 | CG2 | ILE | A | 191 | −5.854 | 47.147 | 15.410 | 1.00 | 222.39 | A | C |
| ATOM | 1386 | C | ILE | A | 191 | −7.376 | 46.894 | 13.074 | 1.00 | 207.21 | A | C |
| ATOM | 1387 | O | ILE | A | 191 | −8.333 | 47.624 | 13.323 | 1.00 | 235.39 | A | O |
| ATOM | 1388 | N | GLN | A | 192 | −6.471 | 47.181 | 12.142 | 1.00 | 204.84 | A | N |
| ATOM | 1389 | CA | GLN | A | 192 | −6.585 | 48.358 | 11.279 | 1.00 | 209.47 | A | C |
| ATOM | 1390 | CB | GLN | A | 192 | −5.226 | 49.023 | 11.060 | 1.00 | 198.36 | A | C |
| ATOM | 1391 | CG | GLN | A | 192 | −4.490 | 49.449 | 12.331 | 1.00 | 201.73 | A | C |
| ATOM | 1392 | CD | GLN | A | 192 | −5.048 | 50.703 | 13.003 | 1.00 | 216.29 | A | C |
| ATOM | 1393 | OE1 | GLN | A | 192 | −5.668 | 51.559 | 12.360 | 1.00 | 207.84 | A | O |
| ATOM | 1394 | NE2 | GLN | A | 192 | −4.798 | 50.834 | 14.308 | 1.00 | 261.94 | A | N |
| ATOM | 1395 | C | GLN | A | 192 | −7.196 | 47.930 | 9.952 | 1.00 | 194.52 | A | C |
| ATOM | 1396 | O | GLN | A | 192 | −7.602 | 48.760 | 9.144 | 1.00 | 205.53 | A | O |
| ATOM | 1397 | N | HIS | A | 193 | −7.252 | 46.621 | 9.728 | 1.00 | 225.76 | A | N |
| ATOM | 1398 | CA | HIS | A | 193 | −8.126 | 46.053 | 8.693 | 1.00 | 240.35 | A | C |
| ATOM | 1399 | CB | HIS | A | 193 | −9.516 | 46.697 | 8.743 | 1.00 | 251.29 | A | C |
| ATOM | 1400 | CG | HIS | A | 193 | −10.163 | 46.629 | 10.112 | 1.00 | 244.54 | A | C |
| ATOM | 1401 | ND1 | HIS | A | 193 | −11.506 | 46.627 | 10.298 | 1.00 | 246.11 | A | N |
| ATOM | 1402 | CE1 | HIS | A | 193 | −11.771 | 46.538 | 11.622 | 1.00 | 295.89 | A | C |
| ATOM | 1403 | NE2 | HIS | A | 193 | −10.603 | 46.498 | 12.277 | 1.00 | 227.95 | A | N |
| ATOM | 1404 | CD2 | HIS | A | 193 | −9.595 | 46.529 | 11.377 | 1.00 | 239.02 | A | C |
| ATOM | 1405 | C | HIS | A | 193 | −7.564 | 46.058 | 7.301 | 1.00 | 208.79 | A | C |
| ATOM | 1406 | O | HIS | A | 193 | −7.556 | 45.018 | 6.640 | 1.00 | 210.91 | A | O |
| ATOM | 1407 | N | TRP | A | 194 | −7.117 | 47.218 | 6.822 | 1.00 | 205.98 | A | N |
| ATOM | 1408 | CA | TRP | A | 194 | −6.343 | 47.236 | 5.580 | 1.00 | 197.14 | A | C |
| ATOM | 1409 | CB | TRP | A | 194 | −7.082 | 47.923 | 4.448 | 1.00 | 202.56 | A | C |
| ATOM | 1410 | CG | TRP | A | 194 | −8.030 | 46.991 | 3.736 | 1.00 | 206.74 | A | C |
| ATOM | 1411 | CD1 | TRP | A | 194 | −9.396 | 46.856 | 3.951 | 1.00 | 190.61 | A | C |
| ATOM | 1412 | NE1 | TRP | A | 194 | −9.933 | 45.902 | 3.122 | 1.00 | 182.62 | A | N |
| ATOM | 1413 | CE2 | TRP | A | 194 | −8.984 | 45.362 | 2.320 | 1.00 | 239.51 | A | C |
| ATOM | 1414 | CD2 | TRP | A | 194 | −7.713 | 46.014 | 2.670 | 1.00 | 230.70 | A | C |
| ATOM | 1415 | CE3 | TRP | A | 194 | −6.559 | 45.639 | 1.994 | 1.00 | 234.76 | A | C |
| ATOM | 1416 | CZ3 | TRP | A | 194 | −6.658 | 44.646 | 0.997 | 1.00 | 206.40 | A | C |
| ATOM | 1417 | CH2 | TRP | A | 194 | −7.883 | 44.028 | 0.679 | 1.00 | 197.51 | A | C |
| ATOM | 1418 | CZ2 | TRP | A | 194 | −9.068 | 44.373 | 1.334 | 1.00 | 235.29 | A | C |
| ATOM | 1419 | C | TRP | A | 194 | −4.993 | 47.830 | 5.775 | 1.00 | 210.27 | A | C |
| ATOM | 1420 | O | TRP | A | 194 | −4.815 | 48.698 | 6.627 | 1.00 | 214.81 | A | O |
| ATOM | 1421 | N | VAL | A | 195 | −4.019 | 47.339 | 5.005 | 1.00 | 231.96 | A | N |
| ATOM | 1422 | CA | VAL | A | 195 | −2.671 | 47.914 | 4.978 | 1.00 | 231.48 | A | C |
| ATOM | 1423 | CB | VAL | A | 195 | −2.732 | 49.402 | 4.532 | 1.00 | 213.25 | A | C |
| ATOM | 1424 | CG1 | VAL | A | 195 | −1.352 | 50.035 | 4.376 | 1.00 | 208.38 | A | C |
| ATOM | 1425 | CG2 | VAL | A | 195 | −3.523 | 49.527 | 3.250 | 1.00 | 221.63 | A | C |
| ATOM | 1426 | C | VAL | A | 195 | −2.017 | 47.812 | 6.365 | 1.00 | 243.32 | A | C |
| ATOM | 1427 | O | VAL | A | 195 | −2.628 | 47.398 | 7.358 | 1.00 | 215.25 | A | O |
| ATOM | 1428 | OXT | VAL | A | 195 | −0.846 | 48.147 | 6.534 | 1.00 | 259.06 | | O |
| ATOM | 1429 | N | MET | B | 1 | −19.457 | 28.398 | 18.812 | 1.00 | 167.16 | A | N |
| ATOM | 1430 | CA | MET | B | 1 | −19.205 | 26.932 | 18.673 | 1.00 | 158.36 | A | C |
| ATOM | 1431 | CB | MET | B | 1 | −18.784 | 26.592 | 17.254 | 1.00 | 173.84 | A | C |
| ATOM | 1432 | CG | MET | B | 1 | −19.946 | 26.535 | 16.279 | 1.00 | 206.83 | A | C |
| ATOM | 1433 | SD | MET | B | 1 | −19.389 | 26.635 | 14.565 | 1.00 | 186.54 | A | S |
| ATOM | 1434 | CE | MET | B | 1 | −18.758 | 24.997 | 14.254 | 1.00 | 201.97 | A | C |
| ATOM | 1435 | C | MET | B | 1 | −18.163 | 26.438 | 19.645 | 1.00 | 176.60 | A | C |
| ATOM | 1436 | O | MET | B | 1 | −17.801 | 25.260 | 19.639 | 1.00 | 187.33 | A | O |
| ATOM | 1437 | N | LEU | B | 2 | −17.715 | 27.344 | 20.504 | 1.00 | 180.09 | A | N |
| ATOM | 1438 | CA | LEU | B | 2 | −16.690 | 27.047 | 21.497 | 1.00 | 183.03 | A | C |
| ATOM | 1439 | CB | LEU | B | 2 | −16.157 | 28.331 | 22.137 | 1.00 | 175.16 | A | C |
| ATOM | 1440 | CG | LEU | B | 2 | −15.416 | 29.256 | 21.166 | 1.00 | 172.51 | A | C |
| ATOM | 1441 | CD1 | LEU | B | 2 | −16.381 | 30.300 | 20.606 | 1.00 | 218.39 | A | C |
| ATOM | 1442 | CD2 | LEU | B | 2 | −14.210 | 29.914 | 21.822 | 1.00 | 208.78 | A | C |
| ATOM | 1443 | C | LEU | B | 2 | −17.191 | 26.084 | 22.557 | 1.00 | 189.65 | A | C |
| ATOM | 1444 | O | LEU | B | 2 | −16.408 | 25.330 | 23.122 | 1.00 | 184.57 | A | O |
| ATOM | 1445 | N | GLY | B | 3 | −18.498 | 26.110 | 22.817 | 1.00 | 217.65 | A | N |
| ATOM | 1446 | CA | GLY | B | 3 | −19.140 | 25.101 | 23.659 | 1.00 | 209.57 | A | C |
| ATOM | 1447 | C | GLY | B | 3 | −18.843 | 23.712 | 23.126 | 1.00 | 182.96 | A | C |
| ATOM | 1448 | O | GLY | B | 3 | −18.424 | 22.822 | 23.873 | 1.00 | 202.42 | A | O |
| ATOM | 1449 | N | LEU | B | 4 | −19.055 | 23.537 | 21.824 | 1.00 | 184.40 | A | N |
| ATOM | 1450 | CA | LEU | B | 4 | −18.713 | 22.301 | 21.152 | 1.00 | 148.44 | A | C |
| ATOM | 1451 | CB | LEU | B | 4 | −19.292 | 22.299 | 19.743 | 1.00 | 165.84 | A | C |
| ATOM | 1452 | CG | LEU | B | 4 | −20.329 | 21.237 | 19.415 | 1.00 | 159.19 | A | C |
| ATOM | 1453 | CD1 | LEU | B | 4 | −20.728 | 21.368 | 17.959 | 1.00 | 139.57 | A | C |
| ATOM | 1454 | CD2 | LEU | B | 4 | −19.756 | 19.855 | 19.663 | 1.00 | 173.38 | A | C |
| ATOM | 1455 | C | LEU | B | 4 | −17.207 | 22.093 | 21.062 | 1.00 | 176.30 | A | C |
| ATOM | 1456 | O | LEU | B | 4 | −16.661 | 21.170 | 21.673 | 1.00 | 166.71 | A | O |
| ATOM | 1457 | N | VAL | B | 5 | −16.544 | 22.959 | 20.297 | 1.00 | 179.36 | A | N |
| ATOM | 1458 | CA | VAL | B | 5 | −15.121 | 22.811 | 20.021 | 1.00 | 162.13 | A | C |
| ATOM | 1459 | CB | VAL | B | 5 | −14.545 | 24.033 | 19.281 | 1.00 | 165.14 | A | C |
| ATOM | 1460 | CG1 | VAL | B | 5 | −13.020 | 23.963 | 19.200 | 1.00 | 160.34 | A | C |
| ATOM | 1461 | CG2 | VAL | B | 5 | −15.163 | 24.143 | 17.895 | 1.00 | 132.50 | A | C |
| ATOM | 1462 | C | VAL | B | 5 | −14.335 | 22.586 | 21.298 | 1.00 | 169.50 | A | C |
| ATOM | 1463 | O | VAL | B | 5 | −13.543 | 21.650 | 21.374 | 1.00 | 182.05 | A | O |
| ATOM | 1464 | N | LEU | B | 6 | −14.569 | 23.434 | 22.298 | 1.00 | 169.80 | A | N |
| ATOM | 1465 | CA | LEU | B | 6 | −13.762 | 23.411 | 23.511 | 1.00 | 166.75 | A | C |
| ATOM | 1466 | CB | LEU | B | 6 | −13.799 | 24.755 | 24.245 | 1.00 | 174.06 | A | C |
| ATOM | 1467 | CG | LEU | B | 6 | −12.979 | 25.880 | 23.599 | 1.00 | 169.99 | A | C |
| ATOM | 1468 | CD1 | LEU | B | 6 | −12.995 | 27.128 | 24.464 | 1.00 | 161.20 | A | C |
| ATOM | 1469 | CD2 | LEU | B | 6 | −11.545 | 25.453 | 23.322 | 1.00 | 198.29 | A | C |
| ATOM | 1470 | C | LEU | B | 6 | −14.120 | 22.272 | 24.436 | 1.00 | 166.07 | A | C |
| ATOM | 1471 | O | LEU | B | 6 | −13.291 | 21.845 | 25.246 | 1.00 | 166.43 | A | O |
| ATOM | 1472 | N | LEU | B | 7 | −15.340 | 21.762 | 24.304 | 1.00 | 147.57 | A | N |
| ATOM | 1473 | CA | LEU | B | 7 | −15.694 | 20.542 | 25.014 | 1.00 | 171.07 | A | C |
| ATOM | 1474 | CB | LEU | B | 7 | −17.106 | 20.092 | 24.661 | 1.00 | 172.69 | A | C |
| ATOM | 1475 | CG | LEU | B | 7 | −17.471 | 18.680 | 25.147 | 1.00 | 204.15 | A | C |
| ATOM | 1476 | CD1 | LEU | B | 7 | −17.352 | 18.543 | 26.665 | 1.00 | 183.26 | A | C |
| ATOM | 1477 | CD2 | LEU | B | 7 | −18.863 | 18.283 | 24.672 | 1.00 | 201.02 | A | C |
| ATOM | 1478 | C | LEU | B | 7 | −14.700 | 19.421 | 24.688 | 1.00 | 171.42 | A | C |
| ATOM | 1479 | O | LEU | B | 7 | −14.243 | 18.679 | 25.575 | 1.00 | 171.77 | A | O |
| ATOM | 1480 | N | TYR | B | 8 | −14.366 | 19.314 | 23.406 | 1.00 | 166.84 | A | N |
| ATOM | 1481 | CA | TYR | B | 8 | −13.530 | 18.234 | 22.925 | 1.00 | 161.85 | A | C |
| ATOM | 1482 | CB | TYR | B | 8 | −13.918 | 17.879 | 21.492 | 1.00 | 140.71 | A | C |
| ATOM | 1483 | CG | TYR | B | 8 | −15.261 | 17.199 | 21.411 | 1.00 | 150.45 | A | C |
| ATOM | 1484 | CD1 | TYR | B | 8 | −15.381 | 15.819 | 21.577 | 1.00 | 160.77 | A | C |
| ATOM | 1485 | CE1 | TYR | B | 8 | −16.617 | 15.183 | 21.518 | 1.00 | 160.09 | A | C |
| ATOM | 1486 | CZ | TYR | B | 8 | −17.759 | 15.942 | 21.302 | 1.00 | 166.68 | A | C |
| ATOM | 1487 | OH | TYR | B | 8 | −18.978 | 15.314 | 21.211 | 1.00 | 179.55 | A | O |
| ATOM | 1488 | CE2 | TYR | B | 8 | −17.663 | 17.318 | 21.140 | 1.00 | 177.51 | A | C |
| ATOM | 1489 | CD2 | TYR | B | 8 | −16.417 | 17.936 | 21.198 | 1.00 | 175.45 | A | C |
| ATOM | 1490 | C | TYR | B | 8 | −12.048 | 18.558 | 23.069 | 1.00 | 154.35 | A | C |
| ATOM | 1491 | O | TYR | B | 8 | −11.235 | 17.656 | 23.299 | 1.00 | 138.88 | A | O |
| ATOM | 1492 | N | VAL | B | 9 | −11.715 | 19.847 | 22.959 | 1.00 | 130.16 | A | N |
| ATOM | 1493 | CA | VAL | B | 9 | −10.379 | 20.337 | 23.273 | 1.00 | 147.03 | A | C |
| ATOM | 1494 | CB | VAL | B | 9 | −10.260 | 21.874 | 23.128 | 1.00 | 180.74 | A | C |
| ATOM | 1495 | CG1 | VAL | B | 9 | −8.987 | 22.407 | 23.791 | 1.00 | 182.53 | A | C |
| ATOM | 1496 | CG2 | VAL | B | 9 | −10.277 | 22.258 | 21.659 | 1.00 | 151.84 | A | C |
| ATOM | 1497 | C | VAL | B | 9 | −10.050 | 19.913 | 24.697 | 1.00 | 174.02 | A | C |
| ATOM | 1498 | O | VAL | B | 9 | −8.881 | 19.704 | 25.039 | 1.00 | 193.41 | A | O |
| ATOM | 1499 | N | GLY | B | 10 | −11.097 | 19.777 | 25.507 | 1.00 | 165.07 | A | N |
| ATOM | 1500 | CA | GLY | B | 10 | −10.977 | 19.220 | 26.830 | 1.00 | 181.97 | A | C |
| ATOM | 1501 | C | GLY | B | 10 | −10.308 | 17.860 | 26.818 | 1.00 | 165.78 | A | C |
| ATOM | 1502 | O | GLY | B | 10 | −9.197 | 17.698 | 27.338 | 1.00 | 210.33 | A | O |
| ATOM | 1503 | N | ILE | B | 11 | −11.006 | 16.876 | 26.288 | 1.00 | 168.60 | A | N |
| ATOM | 1504 | CA | ILE | B | 11 | −10.504 | 15.502 | 26.307 | 1.00 | 174.49 | A | C |
| ATOM | 1505 | CB | ILE | B | 11 | −11.493 | 14.518 | 25.672 | 1.00 | 162.99 | A | C |
| ATOM | 1506 | CG1 | ILE | B | 11 | −12.914 | 15.060 | 25.798 | 1.00 | 167.28 | A | C |
| ATOM | 1507 | CD1 | ILE | B | 11 | −13.837 | 14.706 | 24.664 | 1.00 | 182.06 | A | C |
| ATOM | 1508 | CG2 | ILE | B | 11 | −11.343 | 13.148 | 26.322 | 1.00 | 133.85 | A | C |
| ATOM | 1509 | C | ILE | B | 11 | −9.161 | 15.369 | 25.600 | 1.00 | 176.90 | A | C |
| ATOM | 1510 | O | ILE | B | 11 | −8.223 | 14.796 | 26.160 | 1.00 | 180.05 | A | O |
| ATOM | 1511 | N | VAL | B | 12 | −9.072 | 15.905 | 24.380 | 1.00 | 181.67 | A | N |
| ATOM | 1512 | CA | VAL | B | 12 | −7.859 | 15.779 | 23.568 | 1.00 | 156.79 | A | C |
| ATOM | 1513 | CB | VAL | B | 12 | −8.001 | 16.481 | 22.198 | 1.00 | 152.50 | A | C |
| ATOM | 1514 | CG1 | VAL | B | 12 | −8.098 | 17.984 | 22.357 | 1.00 | 163.18 | A | C |
| ATOM | 1515 | CG2 | VAL | B | 12 | −6.842 | 16.128 | 21.286 | 1.00 | 154.97 | A | C |
| ATOM | 1516 | C | VAL | B | 12 | −6.628 | 16.303 | 24.301 | 1.00 | 165.90 | A | C |
| ATOM | 1517 | O | VAL | B | 12 | −5.529 | 15.743 | 24.153 | 1.00 | 161.02 | A | O |
| ATOM | 1518 | N | LEU | B | 13 | −6.817 | 17.378 | 25.069 | 1.00 | 161.99 | A | N |
| ATOM | 1519 | CA | LEU | B | 13 | −5.749 | 17.927 | 25.896 | 1.00 | 164.78 | A | C |
| ATOM | 1520 | CB | LEU | B | 13 | −6.065 | 19.348 | 26.345 | 1.00 | 187.46 | A | C |
| ATOM | 1521 | CG | LEU | B | 13 | −5.353 | 20.448 | 25.555 | 1.00 | 197.65 | A | C |
| ATOM | 1522 | CD1 | LEU | B | 13 | −5.750 | 21.810 | 26.102 | 1.00 | 215.57 | A | C |
| ATOM | 1523 | CD2 | LEU | B | 13 | −3.836 | 20.268 | 25.598 | 1.00 | 222.78 | A | C |
| ATOM | 1524 | C | LEU | B | 13 | −5.437 | 17.037 | 27.087 | 1.00 | 173.71 | A | C |
| ATOM | 1525 | O | LEU | B | 13 | −4.269 | 16.801 | 27.405 | 1.00 | 194.31 | A | O |
| ATOM | 1526 | N | ILE | B | 14 | −6.481 | 16.534 | 27.735 | 1.00 | 186.01 | A | N |
| ATOM | 1527 | CA | ILE | B | 14 | −6.318 | 15.512 | 28.764 | 1.00 | 203.80 | A | C |
| ATOM | 1528 | CB | ILE | B | 14 | −7.673 | 15.145 | 29.405 | 1.00 | 169.17 | A | C |
| ATOM | 1529 | CG1 | ILE | B | 14 | −8.052 | 16.197 | 30.457 | 1.00 | 231.76 | A | C |
| ATOM | 1530 | CD1 | ILE | B | 14 | −9.383 | 15.980 | 31.144 | 1.00 | 302.25 | A | C |
| ATOM | 1531 | CG2 | ILE | B | 14 | −7.621 | 13.760 | 30.030 | 1.00 | 161.97 | A | C |
| ATOM | 1532 | C | ILE | B | 14 | −5.635 | 14.273 | 28.188 | 1.00 | 188.04 | A | C |
| ATOM | 1533 | O | ILE | B | 14 | −4.701 | 13.741 | 28.789 | 1.00 | 202.63 | A | O |
| ATOM | 1534 | N | SER | B | 15 | −6.101 | 13.832 | 27.022 | 1.00 | 167.75 | A | N |
| ATOM | 1535 | CA | SER | B | 15 | −5.583 | 12.635 | 26.362 | 1.00 | 161.35 | A | C |
| ATOM | 1536 | CB | SER | B | 15 | −6.248 | 12.436 | 24.994 | 1.00 | 174.39 | A | C |
| ATOM | 1537 | OG | SER | B | 15 | −5.528 | 11.496 | 24.208 | 1.00 | 204.10 | A | O |
| ATOM | 1538 | C | SER | B | 15 | −4.068 | 12.728 | 26.226 | 1.00 | 161.88 | A | C |
| ATOM | 1539 | O | SER | B | 15 | −3.353 | 11.860 | 26.704 | 1.00 | 148.81 | A | O |
| ATOM | 1540 | N | ASN | B | 16 | −3.587 | 13.794 | 25.591 | 1.00 | 180.47 | A | N |
| ATOM | 1541 | CA | ASN | B | 16 | −2.149 | 14.013 | 25.409 | 1.00 | 190.94 | A | C |
| ATOM | 1542 | CB | ASN | B | 16 | −1.910 | 15.305 | 24.648 | 1.00 | 159.79 | A | C |
| ATOM | 1543 | CG | ASN | B | 16 | −2.132 | 15.144 | 23.162 | 1.00 | 144.06 | A | C |
| ATOM | 1544 | OD1 | ASN | B | 16 | −1.842 | 14.099 | 22.525 | 1.00 | 177.81 | A | O |
| ATOM | 1545 | ND2 | ASN | B | 16 | −2.640 | 16.211 | 22.580 | 1.00 | 157.24 | A | N |
| ATOM | 1546 | C | ASN | B | 16 | −1.317 | 13.992 | 26.661 | 1.00 | 217.99 | A | C |
| ATOM | 1547 | O | ASN | B | 16 | −0.196 | 13.481 | 26.675 | 1.00 | 220.24 | A | O |
| ATOM | 1548 | N | GLY | B | 17 | −1.886 | 14.557 | 27.702 | 1.00 | 173.78 | A | N |
| ATOM | 1549 | CA | GLY | B | 17 | −1.323 | 14.503 | 29.057 | 1.00 | 171.85 | A | C |
| ATOM | 1550 | C | GLY | B | 17 | −1.126 | 13.085 | 29.538 | 1.00 | 206.33 | A | C |
| ATOM | 1551 | O | GLY | B | 17 | −0.006 | 12.685 | 29.864 | 1.00 | 207.71 | A | O |
| ATOM | 1552 | N | ILE | B | 18 | −2.215 | 12.318 | 29.575 | 1.00 | 193.85 | A | N |
| ATOM | 1553 | CA | ILE | B | 18 | −2.180 | 10.944 | 30.077 | 1.00 | 210.03 | A | C |
| ATOM | 1554 | CB | ILE | B | 18 | −3.578 | 10.299 | 30.080 | 1.00 | 185.15 | A | C |
| ATOM | 1555 | CG1 | ILE | B | 18 | −4.474 | 11.083 | 31.076 | 1.00 | 198.27 | A | C |
| ATOM | 1556 | CD1 | ILE | B | 18 | −5.942 | 10.718 | 31.010 | 1.00 | 205.08 | A | C |
| ATOM | 1557 | CG2 | ILE | B | 18 | −3.514 | 8.835 | 30.493 | 1.00 | 219.44 | A | C |
| ATOM | 1558 | C | ILE | B | 18 | −1.207 | 10.109 | 29.279 | 1.00 | 202.60 | A | C |
| ATOM | 1559 | O | ILE | B | 18 | −0.496 | 9.283 | 29.822 | 1.00 | 219.71 | A | O |
| ATOM | 1560 | N | CYS | B | 19 | −1.166 | 10.338 | 27.976 | 1.00 | 214.30 | A | N |
| ATOM | 1561 | CA | CYS | B | 19 | −0.351 | 9.531 | 27.083 | 1.00 | 246.61 | A | C |
| ATOM | 1562 | CB | CYS | B | 19 | −0.814 | 9.713 | 25.639 | 1.00 | 220.70 | A | C |
| ATOM | 1563 | SG | CYS | B | 19 | −2.495 | 9.091 | 25.327 | 1.00 | 224.12 | A | S |
| ATOM | 1564 | C | CYS | B | 19 | 1.137 | 9.808 | 27.219 | 1.00 | 232.71 | A | C |
| ATOM | 1565 | O | CYS | B | 19 | 1.943 | 8.995 | 26.823 | 1.00 | 234.38 | A | O |
| ATOM | 1566 | N | GLY | B | 20 | 1.500 | 10.955 | 27.784 | 1.00 | 213.69 | A | N |
| ATOM | 1567 | CA | GLY | B | 20 | 2.895 | 11.210 | 28.156 | 1.00 | 221.55 | A | C |
| ATOM | 1568 | C | GLY | B | 20 | 3.228 | 10.515 | 29.467 | 1.00 | 189.60 | A | C |
| ATOM | 1569 | O | GLY | B | 20 | 4.406 | 10.326 | 29.810 | 1.00 | 221.21 | A | O |
| ATOM | 1570 | N | LEU | B | 21 | 2.181 | 10.109 | 30.179 | 1.00 | 211.55 | A | N |
| ATOM | 1571 | CA | LEU | B | 21 | 2.320 | 9.513 | 31.498 | 1.00 | 206.21 | A | C |
| ATOM | 1572 | CB | LEU | B | 21 | 1.248 | 10.037 | 32.452 | 1.00 | 234.02 | A | C |
| ATOM | 1573 | CG | LEU | B | 21 | 1.158 | 11.542 | 32.637 | 1.00 | 222.68 | A | C |
| ATOM | 1574 | CD1 | LEU | B | 21 | −0.141 | 11.833 | 33.366 | 1.00 | 226.16 | A | C |
| ATOM | 1575 | CD2 | LEU | B | 21 | 2.385 | 12.058 | 33.375 | 1.00 | 242.87 | A | C |
| ATOM | 1576 | C | LEU | B | 21 | 2.265 | 8.009 | 31.447 | 1.00 | 202.86 | A | C |
| ATOM | 1577 | O | LEU | B | 21 | 3.277 | 7.383 | 31.708 | 1.00 | 236.09 | A | O |
| ATOM | 1578 | N | THR | B | 22 | 1.103 | 7.426 | 31.107 | 1.00 | 225.96 | A | N |
| ATOM | 1579 | CA | THR | B | 22 | 0.996 | 5.962 | 30.918 | 1.00 | 240.46 | A | C |
| ATOM | 1580 | CB | THR | B | 22 | −0.462 | 5.425 | 30.805 | 1.00 | 184.03 | A | C |
| ATOM | 1581 | OG1 | THR | B | 22 | −1.258 | 6.275 | 29.950 | 1.00 | 210.27 | A | O |
| ATOM | 1582 | CG2 | THR | B | 22 | −1.117 | 5.297 | 32.168 | 1.00 | 222.38 | A | C |
| ATOM | 1583 | C | THR | B | 22 | 1.866 | 5.501 | 29.756 | 1.00 | 226.25 | A | C |
| ATOM | 1584 | O | THR | B | 22 | 2.280 | 4.337 | 29.711 | 1.00 | 227.23 | A | O |
| ATOM | 1585 | N | LYS | B | 23 | 2.139 | 6.437 | 28.839 | 1.00 | 247.55 | A | N |
| ATOM | 1586 | CA | LYS | B | 23 | 3.054 | 6.295 | 27.695 | 1.00 | 223.30 | A | C |
| ATOM | 1587 | CB | LYS | B | 23 | 4.520 | 6.331 | 28.098 | 1.00 | 214.67 | A | C |
| ATOM | 1588 | CG | LYS | B | 23 | 5.006 | 7.769 | 28.116 | 1.00 | 213.08 | A | C |
| ATOM | 1589 | CD | LYS | B | 23 | 6.497 | 7.816 | 28.346 | 1.00 | 197.04 | A | C |
| ATOM | 1590 | CE | LYS | B | 23 | 6.811 | 8.133 | 29.789 | 1.00 | 281.60 | A | C |
| ATOM | 1591 | NZ | LYS | B | 23 | 8.162 | 8.730 | 29.759 | 1.00 | 324.42 | A | N |
| ATOM | 1592 | C | LYS | B | 23 | 2.722 | 5.234 | 26.669 | 1.00 | 181.13 | A | C |
| ATOM | 1593 | O | LYS | B | 23 | 3.293 | 4.120 | 26.642 | 1.00 | 192.89 | A | O |
| ATOM | 1594 | N | VAL | B | 24 | 1.808 | 5.623 | 25.788 | 1.00 | 152.44 | A | N |
| ATOM | 1595 | CA | VAL | B | 24 | 1.207 | 4.679 | 24.907 | 1.00 | 148.78 | A | C |
| ATOM | 1596 | CB | VAL | B | 24 | −0.335 | 4.648 | 25.096 | 1.00 | 159.81 | A | C |
| ATOM | 1597 | CG1 | VAL | B | 24 | −0.934 | 6.015 | 24.964 | 1.00 | 182.67 | A | C |
| ATOM | 1598 | CG2 | VAL | B | 24 | −0.994 | 3.703 | 24.117 | 1.00 | 194.57 | A | C |
| ATOM | 1599 | C | VAL | B | 24 | 1.641 | 4.928 | 23.469 | 1.00 | 151.95 | A | C |
| ATOM | 1600 | O | VAL | B | 24 | 1.837 | 6.075 | 23.052 | 1.00 | 165.23 | A | O |
| ATOM | 1601 | N | ASP | B | 25 | 1.919 | 3.820 | 22.789 | 1.00 | 146.02 | A | N |
| ATOM | 1602 | CA | ASP | B | 25 | 1.718 | 3.643 | 21.362 | 1.00 | 156.26 | A | C |
| ATOM | 1603 | CB | ASP | B | 25 | 0.704 | 2.514 | 21.226 | 1.00 | 181.99 | A | C |
| ATOM | 1604 | CG | ASP | B | 25 | 0.283 | 2.248 | 19.804 | 1.00 | 210.90 | A | C |
| ATOM | 1605 | OD1 | ASP | B | 25 | 1.069 | 1.674 | 19.029 | 1.00 | 239.18 | A | O |
| ATOM | 1606 | OD2 | ASP | B | 25 | −0.871 | 2.568 | 19.462 | 1.00 | 224.85 | A | O |
| ATOM | 1607 | C | ASP | B | 25 | 1.276 | 4.913 | 20.617 | 1.00 | 149.79 | A | C |
| ATOM | 1608 | O | ASP | B | 25 | 0.085 | 5.204 | 20.516 | 1.00 | 167.62 | A | O |
| ATOM | 1609 | N | PRO | B | 26 | 2.241 | 5.664 | 20.063 | 1.00 | 119.15 | A | N |
| ATOM | 1610 | CA | PRO | B | 26 | 1.967 | 6.943 | 19.400 | 1.00 | 114.89 | A | C |
| ATOM | 1611 | CB | PRO | B | 26 | 3.265 | 7.227 | 18.638 | 1.00 | 122.10 | A | C |
| ATOM | 1612 | CG | PRO | B | 26 | 4.296 | 6.457 | 19.380 | 1.00 | 115.88 | A | C |
| ATOM | 1613 | CD | PRO | B | 26 | 3.602 | 5.192 | 19.765 | 1.00 | 138.43 | A | C |
| ATOM | 1614 | C | PRO | B | 26 | 0.824 | 6.897 | 18.391 | 1.00 | 123.33 | A | C |
| ATOM | 1615 | O | PRO | B | 26 | 0.174 | 7.916 | 18.165 | 1.00 | 140.97 | A | O |
| ATOM | 1616 | N | LYS | B | 27 | 0.606 | 5.748 | 17.745 | 1.00 | 143.72 | A | N |
| ATOM | 1617 | CA | LYS | B | 27 | −0.516 | 5.588 | 16.861 | 1.00 | 113.66 | A | C |
| ATOM | 1618 | CB | LYS | B | 27 | −0.528 | 4.191 | 16.215 | 1.00 | 127.89 | A | C |
| ATOM | 1619 | CG | LYS | B | 27 | 0.775 | 3.708 | 15.543 | 1.00 | 152.45 | A | C |
| ATOM | 1620 | CD | LYS | B | 27 | 0.842 | 4.102 | 14.068 | 1.00 | 119.18 | A | C |
| ATOM | 1621 | CE | LYS | B | 27 | 1.801 | 3.221 | 13.283 | 1.00 | 168.71 | A | C |
| ATOM | 1622 | NZ | LYS | B | 27 | 1.858 | 3.518 | 11.830 | 1.00 | 178.53 | A | N |
| ATOM | 1623 | C | LYS | B | 27 | −1.834 | 5.831 | 17.593 | 1.00 | 126.48 | A | C |
| ATOM | 1624 | O | LYS | B | 27 | −2.729 | 6.469 | 17.051 | 1.00 | 123.59 | A | O |
| ATOM | 1625 | N | SER | B | 28 | −1.957 | 5.325 | 18.807 | 1.00 | 141.52 | A | N |
| ATOM | 1626 | CA | SER | B | 28 | −3.224 | 5.362 | 19.535 | 1.00 | 163.01 | A | C |
| ATOM | 1627 | CB | SER | B | 28 | −3.206 | 4.411 | 20.734 | 1.00 | 168.62 | A | C |
| ATOM | 1628 | OG | SER | B | 28 | −2.879 | 3.103 | 20.382 | 1.00 | 196.78 | A | O |
| ATOM | 1629 | C | SER | B | 28 | −3.563 | 6.755 | 20.011 | 1.00 | 147.56 | A | C |
| ATOM | 1630 | O | SER | B | 28 | −4.701 | 7.208 | 19.844 | 1.00 | 157.78 | A | O |
| ATOM | 1631 | N | THR | B | 29 | −2.578 | 7.432 | 20.592 | 1.00 | 128.71 | A | N |
| ATOM | 1632 | CA | THR | B | 29 | −2.765 | 8.804 | 21.018 | 1.00 | 143.99 | A | C |
| ATOM | 1633 | CB | THR | B | 29 | −1.445 | 9.450 | 21.497 | 1.00 | 163.73 | A | C |
| ATOM | 1634 | OG1 | THR | B | 29 | −0.638 | 9.766 | 20.364 | 1.00 | 175.46 | A | O |
| ATOM | 1635 | CG2 | THR | B | 29 | −0.655 | 8.525 | 22.391 | 1.00 | 145.51 | A | C |
| ATOM | 1636 | C | THR | B | 29 | −3.327 | 9.639 | 19.863 | 1.00 | 131.50 | A | C |
| ATOM | 1637 | O | THR | B | 29 | −4.256 | 10.436 | 20.044 | 1.00 | 163.20 | A | O |
| ATOM | 1638 | N | ALA | B | 30 | −2.761 | 9.438 | 18.680 | 1.00 | 107.95 | A | N |
| ATOM | 1639 | CA | ALA | B | 30 | −3.041 | 10.294 | 17.540 | 1.00 | 126.54 | A | C |
| ATOM | 1640 | CB | ALA | B | 30 | −2.191 | 9.885 | 16.337 | 1.00 | 101.74 | A | C |
| ATOM | 1641 | C | ALA | B | 30 | −4.520 | 10.295 | 17.187 | 1.00 | 131.49 | A | C |
| ATOM | 1642 | O | ALA | B | 30 | −5.075 | 11.331 | 16.826 | 1.00 | 124.57 | A | O |
| ATOM | 1643 | N | VAL | B | 31 | −5.153 | 9.133 | 17.311 | 1.00 | 122.21 | A | N |
| ATOM | 1644 | CA | VAL | B | 31 | −6.572 | 8.985 | 17.026 | 1.00 | 126.41 | A | C |
| ATOM | 1645 | CB | VAL | B | 31 | −7.149 | 7.722 | 17.674 | 1.00 | 126.87 | A | C |
| ATOM | 1646 | CG1 | VAL | B | 31 | −8.631 | 7.612 | 17.363 | 1.00 | 121.81 | A | C |
| ATOM | 1647 | CG2 | VAL | B | 31 | −6.420 | 6.492 | 17.174 | 1.00 | 134.74 | A | C |
| ATOM | 1648 | C | VAL | B | 31 | −7.373 | 10.163 | 17.544 | 1.00 | 124.26 | A | C |
| ATOM | 1649 | O | VAL | B | 31 | −8.087 | 10.827 | 16.780 | 1.00 | 137.64 | A | O |
| ATOM | 1650 | N | MET | B | 32 | −7.254 | 10.410 | 18.843 | 1.00 | 110.08 | A | N |
| ATOM | 1651 | CA | MET | B | 32 | −8.000 | 11.486 | 19.456 | 1.00 | 128.13 | A | C |
| ATOM | 1652 | CB | MET | B | 32 | −7.757 | 11.512 | 20.955 | 1.00 | 133.40 | A | C |
| ATOM | 1653 | CG | MET | B | 32 | −8.642 | 12.517 | 21.669 | 1.00 | 165.90 | A | C |
| ATOM | 1654 | SD | MET | B | 32 | −10.318 | 12.620 | 20.986 | 1.00 | 185.30 | A | S |
| ATOM | 1655 | CE | MET | B | 32 | −11.116 | 11.224 | 21.767 | 1.00 | 159.06 | A | C |
| ATOM | 1656 | C | MET | B | 32 | −7.649 | 12.822 | 18.813 | 1.00 | 131.18 | A | C |
| ATOM | 1657 | O | MET | B | 32 | −8.528 | 13.557 | 18.348 | 1.00 | 128.46 | A | O |
| ATOM | 1658 | N | ASN | B | 33 | −6.354 | 13.106 | 18.750 | 1.00 | 121.39 | A | N |
| ATOM | 1659 | CA | ASN | B | 33 | −5.870 | 14.308 | 18.084 | 1.00 | 114.62 | A | C |
| ATOM | 1660 | CB | ASN | B | 33 | −4.349 | 14.305 | 18.019 | 1.00 | 116.96 | A | C |
| ATOM | 1661 | CG | ASN | B | 33 | −3.721 | 14.319 | 19.388 | 1.00 | 120.19 | A | C |
| ATOM | 1662 | OD1 | ASN | B | 33 | −3.627 | 15.360 | 20.023 | 1.00 | 120.29 | A | O |
| ATOM | 1663 | ND2 | ASN | B | 33 | −3.276 | 13.161 | 19.840 | 1.00 | 137.12 | A | N |
| ATOM | 1664 | C | ASN | B | 33 | −6.469 | 14.534 | 16.699 | 1.00 | 112.84 | A | C |
| ATOM | 1665 | O | ASN | B | 33 | −6.721 | 15.668 | 16.317 | 1.00 | 111.71 | A | O |
| ATOM | 1666 | N | PHE | B | 34 | −6.717 | 13.444 | 15.982 | 1.00 | 112.72 | A | N |
| ATOM | 1667 | CA | PHE | B | 34 | −7.338 | 13.501 | 14.656 | 1.00 | 118.12 | A | C |
| ATOM | 1668 | CB | PHE | B | 34 | −7.289 | 12.130 | 13.973 | 1.00 | 116.95 | A | C |
| ATOM | 1669 | CG | PHE | B | 34 | −6.036 | 11.880 | 13.175 | 1.00 | 107.39 | A | C |
| ATOM | 1670 | CD1 | PHE | B | 34 | −5.780 | 12.597 | 12.012 | 1.00 | 119.67 | A | C |
| ATOM | 1671 | CE1 | PHE | B | 34 | −4.638 | 12.360 | 11.263 | 1.00 | 128.12 | A | C |
| ATOM | 1672 | CZ | PHE | B | 34 | −3.749 | 11.383 | 11.664 | 1.00 | 102.86 | A | C |
| ATOM | 1673 | CE2 | PHE | B | 34 | −3.995 | 10.655 | 12.805 | 1.00 | 105.79 | A | C |
| ATOM | 1674 | CD2 | PHE | B | 34 | −5.137 | 10.896 | 13.555 | 1.00 | 113.57 | A | C |
| ATOM | 1675 | C | PHE | B | 34 | −8.783 | 13.970 | 14.734 | 1.00 | 119.59 | A | C |
| ATOM | 1676 | O | PHE | B | 34 | −9.194 | 14.894 | 14.011 | 1.00 | 112.74 | A | O |
| ATOM | 1677 | N | PHE | B | 35 | −9.549 | 13.324 | 15.608 | 1.00 | 130.76 | A | N |
| ATOM | 1678 | CA | PHE | B | 35 | −10.961 | 13.632 | 15.763 | 1.00 | 134.73 | A | C |
| ATOM | 1679 | CB | PHE | B | 35 | −11.573 | 12.871 | 16.938 | 1.00 | 124.85 | A | C |
| ATOM | 1680 | CG | PHE | B | 35 | −11.814 | 11.431 | 16.651 | 1.00 | 130.05 | A | C |
| ATOM | 1681 | CD1 | PHE | B | 35 | −12.168 | 11.025 | 15.368 | 1.00 | 122.38 | A | C |
| ATOM | 1682 | CE1 | PHE | B | 35 | −12.388 | 9.694 | 15.088 | 1.00 | 125.63 | A | C |
| ATOM | 1683 | CZ | PHE | B | 35 | −12.275 | 8.753 | 16.100 | 1.00 | 153.65 | A | C |
| ATOM | 1684 | CE2 | PHE | B | 35 | −11.927 | 9.146 | 17.383 | 1.00 | 141.96 | A | C |
| ATOM | 1685 | CD2 | PHE | B | 35 | −11.700 | 10.482 | 17.653 | 1.00 | 133.85 | A | C |
| ATOM | 1686 | C | PHE | B | 35 | −11.142 | 15.088 | 16.009 | 1.00 | 114.97 | A | C |
| ATOM | 1687 | O | PHE | B | 35 | −11.921 | 15.739 | 15.338 | 1.00 | 116.08 | A | O |
| ATOM | 1688 | N | VAL | B | 36 | −10.391 | 15.590 | 16.973 | 1.00 | 115.32 | A | N |
| ATOM | 1689 | CA | VAL | B | 36 | −10.580 | 16.941 | 17.447 | 1.00 | 113.00 | A | C |
| ATOM | 1690 | CB | VAL | B | 36 | −9.918 | 17.154 | 18.811 | 1.00 | 118.29 | A | C |
| ATOM | 1691 | CG1 | VAL | B | 36 | −9.852 | 18.635 | 19.150 | 1.00 | 107.12 | A | C |
| ATOM | 1692 | CG2 | VAL | B | 36 | −10.693 | 16.387 | 19.862 | 1.00 | 103.29 | A | C |
| ATOM | 1693 | C | VAL | B | 36 | −10.079 | 17.937 | 16.426 | 1.00 | 113.72 | A | C |
| ATOM | 1694 | O | VAL | B | 36 | −10.846 | 18.779 | 15.965 | 1.00 | 101.99 | A | O |
| ATOM | 1695 | N | GLY | B | 37 | −8.802 | 17.837 | 16.081 | 1.00 | 106.12 | A | N |
| ATOM | 1696 | CA | GLY | B | 37 | −8.250 | 18.612 | 14.981 | 1.00 | 112.02 | A | C |
| ATOM | 1697 | C | GLY | B | 37 | −9.178 | 18.666 | 13.772 | 1.00 | 126.10 | A | C |
| ATOM | 1698 | O | GLY | B | 37 | −9.466 | 19.753 | 13.230 | 1.00 | 114.11 | A | O |
| ATOM | 1699 | N | GLY | B | 38 | −9.656 | 17.491 | 13.359 | 1.00 | 107.21 | A | N |
| ATOM | 1700 | CA | GLY | B | 38 | −10.613 | 17.397 | 12.261 | 1.00 | 106.18 | A | C |
| ATOM | 1701 | C | GLY | B | 38 | −11.838 | 18.229 | 12.566 | 1.00 | 112.71 | A | C |
| ATOM | 1702 | O | GLY | B | 38 | −12.263 | 19.052 | 11.765 | 1.00 | 118.87 | A | O |
| ATOM | 1703 | N | LEU | B | 39 | −12.401 | 18.015 | 13.743 | 1.00 | 131.36 | A | N |
| ATOM | 1704 | CA | LEU | B | 39 | −13.552 | 18.781 | 14.175 | 1.00 | 129.81 | A | C |
| ATOM | 1705 | CB | LEU | B | 39 | −13.923 | 18.394 | 15.601 | 1.00 | 130.46 | A | C |
| ATOM | 1706 | CG | LEU | B | 39 | −15.198 | 19.044 | 16.138 | 1.00 | 158.86 | A | C |
| ATOM | 1707 | CD1 | LEU | B | 39 | −16.385 | 18.776 | 15.212 | 1.00 | 122.69 | A | C |
| ATOM | 1708 | CD2 | LEU | B | 39 | −15.491 | 18.565 | 17.566 | 1.00 | 137.57 | A | C |
| ATOM | 1709 | C | LEU | B | 39 | −13.317 | 20.294 | 14.073 | 1.00 | 130.69 | A | C |
| ATOM | 1710 | O | LEU | B | 39 | −14.071 | 21.001 | 13.416 | 1.00 | 121.89 | A | O |
| ATOM | 1711 | N | SER | B | 40 | −12.261 | 20.787 | 14.706 | 1.00 | 129.26 | A | N |
| ATOM | 1712 | CA | SER | B | 40 | −11.944 | 22.220 | 14.675 | 1.00 | 121.04 | A | C |
| ATOM | 1713 | CB | SER | B | 40 | −10.540 | 22.471 | 15.205 | 1.00 | 129.63 | A | C |
| ATOM | 1714 | OG | SER | B | 40 | −10.139 | 21.450 | 16.105 | 1.00 | 140.93 | A | O |
| ATOM | 1715 | C | SER | B | 40 | −12.005 | 22.747 | 13.256 | 1.00 | 112.07 | A | C |
| ATOM | 1716 | O | SER | B | 40 | −12.639 | 23.751 | 12.976 | 1.00 | 114.01 | A | O |
| ATOM | 1717 | N | ILE | B | 41 | −11.347 | 22.047 | 12.360 | 1.00 | 92.36 | A | N |
| ATOM | 1718 | CA | ILE | B | 41 | −11.128 | 22.572 | 11.023 | 1.00 | 103.93 | A | C |
| ATOM | 1719 | CB | ILE | B | 41 | −10.024 | 21.785 | 10.312 | 1.00 | 106.17 | A | C |
| ATOM | 1720 | CG1 | ILE | B | 41 | −8.710 | 22.086 | 11.045 | 1.00 | 122.80 | A | C |
| ATOM | 1721 | CD1 | ILE | B | 41 | −7.633 | 21.057 | 10.823 | 1.00 | 103.29 | A | C |
| ATOM | 1722 | CG2 | ILE | B | 41 | −9.994 | 22.064 | 8.798 | 1.00 | 102.59 | A | C |
| ATOM | 1723 | C | ILE | B | 41 | −12.399 | 22.632 | 10.205 | 1.00 | 121.25 | A | C |
| ATOM | 1724 | O | ILE | B | 41 | −12.617 | 23.589 | 9.459 | 1.00 | 112.33 | A | O |
| ATOM | 1725 | N | VAL | B | 42 | −13.228 | 21.611 | 10.370 | 1.00 | 120.64 | A | N |
| ATOM | 1726 | CA | VAL | B | 42 | −14.558 | 21.631 | 9.816 | 1.00 | 116.72 | A | C |
| ATOM | 1727 | CB | VAL | B | 42 | −15.371 | 20.394 | 10.212 | 1.00 | 111.40 | A | C |
| ATOM | 1728 | CG1 | VAL | B | 42 | −16.654 | 20.370 | 9.406 | 1.00 | 132.47 | A | C |
| ATOM | 1729 | CG2 | VAL | B | 42 | −14.586 | 19.120 | 9.980 | 1.00 | 121.52 | A | C |
| ATOM | 1730 | C | VAL | B | 42 | −15.296 | 22.852 | 10.340 | 1.00 | 122.53 | A | C |
| ATOM | 1731 | O | VAL | B | 42 | −15.785 | 23.673 | 9.558 | 1.00 | 135.02 | A | O |
| ATOM | 1732 | N | CYS | B | 43 | −15.364 | 22.970 | 11.661 | 1.00 | 111.31 | A | N |
| ATOM | 1733 | CA | CYS | B | 43 | −16.115 | 24.050 | 12.288 | 1.00 | 118.30 | A | C |
| ATOM | 1734 | CB | CYS | B | 43 | −15.874 | 24.062 | 13.789 | 1.00 | 118.15 | A | C |
| ATOM | 1735 | SG | CYS | B | 43 | −16.490 | 22.577 | 14.588 | 1.00 | 136.62 | A | S |
| ATOM | 1736 | C | CYS | B | 43 | −15.726 | 25.388 | 11.703 | 1.00 | 132.87 | A | C |
| ATOM | 1737 | O | CYS | B | 43 | −16.545 | 26.071 | 11.087 | 1.00 | 129.60 | A | O |
| ATOM | 1738 | N | ASN | B | 44 | −14.451 | 25.724 | 11.864 | 1.00 | 120.02 | A | N |
| ATOM | 1739 | CA | ASN | B | 44 | −13.938 | 27.003 | 11.427 | 1.00 | 131.67 | A | C |
| ATOM | 1740 | CB | ASN | B | 44 | −12.465 | 27.133 | 11.784 | 1.00 | 134.85 | A | C |
| ATOM | 1741 | CG | ASN | B | 44 | −12.226 | 27.130 | 13.292 | 1.00 | 154.54 | A | C |
| ATOM | 1742 | OD1 | ASN | B | 44 | −11.126 | 26.828 | 13.759 | 1.00 | 155.85 | A | O |
| ATOM | 1743 | ND2 | ASN | B | 44 | −13.263 | 27.460 | 14.063 | 1.00 | 173.95 | A | N |
| ATOM | 1744 | C | ASN | B | 44 | −14.183 | 27.247 | 9.945 | 1.00 | 124.16 | A | C |
| ATOM | 1745 | O | ASN | B | 44 | −14.228 | 28.386 | 9.509 | 1.00 | 124.40 | A | O |
| ATOM | 1746 | N | VAL | B | 45 | −14.396 | 26.176 | 9.191 | 1.00 | 111.83 | A | N |
| ATOM | 1747 | CA | VAL | B | 45 | −14.668 | 26.297 | 7.771 | 1.00 | 114.68 | A | C |
| ATOM | 1748 | CB | VAL | B | 45 | −14.360 | 25.002 | 7.032 | 1.00 | 113.18 | A | C |
| ATOM | 1749 | CG1 | VAL | B | 45 | −15.280 | 24.833 | 5.836 | 1.00 | 118.82 | A | C |
| ATOM | 1750 | CG2 | VAL | B | 45 | −12.914 | 25.035 | 6.598 | 1.00 | 116.45 | A | C |
| ATOM | 1751 | C | VAL | B | 45 | −16.074 | 26.801 | 7.458 | 1.00 | 132.23 | A | C |
| ATOM | 1752 | O | VAL | B | 45 | −16.247 | 27.687 | 6.602 | 1.00 | 129.39 | A | O |
| ATOM | 1753 | N | VAL | B | 46 | −17.069 | 26.239 | 8.144 | 1.00 | 135.14 | A | N |
| ATOM | 1754 | CA | VAL | B | 46 | −18.421 | 26.788 | 8.069 | 1.00 | 155.73 | A | C |
| ATOM | 1755 | CB | VAL | B | 46 | −19.468 | 25.923 | 8.822 | 1.00 | 144.13 | A | C |
| ATOM | 1756 | CG1 | VAL | B | 46 | −19.158 | 24.446 | 8.640 | 1.00 | 150.36 | A | C |
| ATOM | 1757 | CG2 | VAL | B | 46 | −19.502 | 26.238 | 10.298 | 1.00 | 142.80 | A | C |
| ATOM | 1758 | C | VAL | B | 46 | −18.364 | 28.216 | 8.612 | 1.00 | 132.99 | A | C |
| ATOM | 1759 | O | VAL | B | 46 | −18.887 | 29.151 | 8.017 | 1.00 | 136.66 | A | O |
| ATOM | 1760 | N | VAL | B | 47 | −17.660 | 28.385 | 9.715 | 1.00 | 120.16 | A | N |
| ATOM | 1761 | CA | VAL | B | 47 | −17.537 | 29.685 | 10.345 | 1.00 | 121.34 | A | C |
| ATOM | 1762 | CB | VAL | B | 47 | −16.778 | 29.566 | 11.673 | 1.00 | 128.99 | A | C |
| ATOM | 1763 | CG1 | VAL | B | 47 | −16.368 | 30.917 | 12.190 | 1.00 | 149.10 | A | C |
| ATOM | 1764 | CG2 | VAL | B | 47 | −17.633 | 28.851 | 12.703 | 1.00 | 142.29 | A | C |
| ATOM | 1765 | C | VAL | B | 47 | −16.842 | 30.676 | 9.422 | 1.00 | 132.79 | A | C |
| ATOM | 1766 | O | VAL | B | 47 | −17.252 | 31.823 | 9.344 | 1.00 | 149.11 | A | O |
| ATOM | 1767 | N | ILE | B | 48 | −15.796 | 30.236 | 8.729 | 1.00 | 130.54 | A | N |
| ATOM | 1768 | CA | ILE | B | 48 | −15.158 | 31.066 | 7.713 | 1.00 | 133.31 | A | C |
| ATOM | 1769 | CB | ILE | B | 48 | −13.947 | 30.381 | 7.067 | 1.00 | 129.49 | A | C |
| ATOM | 1770 | CG1 | ILE | B | 48 | −12.753 | 30.436 | 8.009 | 1.00 | 125.78 | A | C |
| ATOM | 1771 | CD1 | ILE | B | 48 | −11.737 | 29.369 | 7.716 | 1.00 | 137.34 | A | C |
| ATOM | 1772 | CG2 | ILE | B | 48 | −13.577 | 31.065 | 5.761 | 1.00 | 139.03 | A | C |
| ATOM | 1773 | C | ILE | B | 48 | −16.157 | 31.392 | 6.625 | 1.00 | 137.13 | A | C |
| ATOM | 1774 | O | ILE | B | 48 | −16.264 | 32.538 | 6.212 | 1.00 | 158.45 | A | O |
| ATOM | 1775 | N | THR | B | 49 | −16.885 | 30.385 | 6.161 | 1.00 | 136.86 | A | N |
| ATOM | 1776 | CA | THR | B | 49 | −17.842 | 30.615 | 5.085 | 1.00 | 152.99 | A | C |
| ATOM | 1777 | CB | THR | B | 49 | −18.380 | 29.297 | 4.479 | 1.00 | 129.80 | A | C |
| ATOM | 1778 | OG1 | THR | B | 49 | −18.948 | 28.486 | 5.511 | 1.00 | 132.86 | A | O |
| ATOM | 1779 | CG2 | THR | B | 49 | −17.275 | 28.512 | 3.809 | 1.00 | 136.34 | A | C |
| ATOM | 1780 | C | THR | B | 49 | −18.998 | 31.533 | 5.537 | 1.00 | 162.78 | A | C |
| ATOM | 1781 | O | THR | B | 49 | −19.531 | 32.305 | 4.738 | 1.00 | 166.92 | A | O |
| ATOM | 1782 | N | TYR | B | 50 | −19.359 | 31.459 | 6.820 | 1.00 | 157.71 | A | N |
| ATOM | 1783 | CA | TYR | B | 50 | −20.491 | 32.239 | 7.357 | 1.00 | 180.76 | A | C |
| ATOM | 1784 | CB | TYR | B | 50 | −20.918 | 31.757 | 8.745 | 1.00 | 196.95 | A | C |
| ATOM | 1785 | CG | TYR | B | 50 | −21.953 | 30.670 | 8.668 | 1.00 | 257.36 | A | C |
| ATOM | 1786 | CD1 | TYR | B | 50 | −21.565 | 29.319 | 8.646 | 1.00 | 207.64 | A | C |
| ATOM | 1787 | CE1 | TYR | B | 50 | −22.515 | 28.312 | 8.546 | 1.00 | 268.66 | A | C |
| ATOM | 1788 | CZ | TYR | B | 50 | −23.877 | 28.655 | 8.487 | 1.00 | 311.05 | A | C |
| ATOM | 1789 | OH | TYR | B | 50 | −24.821 | 27.659 | 8.396 | 1.00 | 395.61 | A | O |
| ATOM | 1790 | CE2 | TYR | B | 50 | −24.284 | 29.987 | 8.512 | 1.00 | 277.76 | A | C |
| ATOM | 1791 | CD2 | TYR | B | 50 | −23.324 | 30.984 | 8.596 | 1.00 | 273.97 | A | C |
| ATOM | 1792 | C | TYR | B | 50 | −20.266 | 33.729 | 7.416 | 1.00 | 179.53 | A | C |
| ATOM | 1793 | O | TYR | B | 50 | −21.221 | 34.510 | 7.317 | 1.00 | 202.84 | A | O |
| ATOM | 1794 | N | SER | B | 51 | −19.013 | 34.113 | 7.618 | 1.00 | 155.01 | A | N |
| ATOM | 1795 | CA | SER | B | 51 | −18.638 | 35.513 | 7.529 | 1.00 | 180.93 | A | C |
| ATOM | 1796 | CB | SER | B | 51 | −17.498 | 35.849 | 8.491 | 1.00 | 154.81 | A | C |
| ATOM | 1797 | OG | SER | B | 51 | −16.442 | 34.936 | 8.364 | 1.00 | 163.86 | A | O |
| ATOM | 1798 | C | SER | B | 51 | −18.305 | 35.884 | 6.084 | 1.00 | 180.36 | A | C |
| ATOM | 1799 | O | SER | B | 51 | −18.609 | 36.988 | 5.641 | 1.00 | 203.63 | A | O |
| ATOM | 1800 | N | ALA | B | 52 | −17.708 | 34.949 | 5.349 | 1.00 | 154.34 | A | N |
| ATOM | 1801 | CA | ALA | B | 52 | −17.603 | 35.069 | 3.906 | 1.00 | 161.60 | A | C |
| ATOM | 1802 | CB | ALA | B | 52 | −16.995 | 33.808 | 3.323 | 1.00 | 153.27 | A | C |
| ATOM | 1803 | C | ALA | B | 52 | −18.988 | 35.345 | 3.301 | 1.00 | 190.86 | A | C |
| ATOM | 1804 | O | ALA | B | 52 | −19.090 | 36.014 | 2.261 | 1.00 | 205.80 | A | O |
| ATOM | 1805 | N | LEU | B | 53 | −20.036 | 34.830 | 3.960 | 1.00 | 194.30 | A | N |
| ATOM | 1806 | CA | LEU | B | 53 | −21.435 | 35.152 | 3.639 | 1.00 | 201.75 | A | C |
| ATOM | 1807 | CB | LEU | B | 53 | −22.393 | 34.307 | 4.474 | 1.00 | 219.89 | A | C |
| ATOM | 1808 | CG | LEU | B | 53 | −23.062 | 33.070 | 3.896 | 1.00 | 266.98 | A | C |
| ATOM | 1809 | CD1 | LEU | B | 53 | −23.229 | 32.050 | 5.017 | 1.00 | 256.39 | A | C |
| ATOM | 1810 | CD2 | LEU | B | 53 | −24.398 | 33.434 | 3.236 | 1.00 | 294.08 | A | C |
| ATOM | 1811 | C | LEU | B | 53 | −21.765 | 36.599 | 3.925 | 1.00 | 199.75 | A | C |
| ATOM | 1812 | O | LEU | B | 53 | −22.276 | 37.306 | 3.061 | 1.00 | 205.93 | A | O |
| ATOM | 1813 | N | HIS | B | 54 | −21.481 | 37.012 | 5.156 | 1.00 | 185.40 | A | N |
| ATOM | 1814 | CA | HIS | B | 54 | −21.919 | 38.293 | 5.662 | 1.00 | 202.83 | A | C |
| ATOM | 1815 | CB | HIS | B | 54 | −22.750 | 38.103 | 6.929 | 1.00 | 236.13 | A | C |
| ATOM | 1816 | CG | HIS | B | 54 | −23.842 | 37.063 | 6.791 | 1.00 | 238.21 | A | C |
| ATOM | 1817 | ND1 | HIS | B | 54 | −23.759 | 35.833 | 7.352 | 1.00 | 216.89 | A | N |
| ATOM | 1818 | CE1 | HIS | B | 54 | −24.868 | 35.133 | 7.052 | 1.00 | 211.00 | A | C |
| ATOM | 1819 | NE2 | HIS | B | 54 | −25.660 | 35.914 | 6.295 | 1.00 | 237.26 | A | N |
| ATOM | 1820 | CD2 | HIS | B | 54 | −25.052 | 37.104 | 6.109 | 1.00 | 248.65 | A | C |
| ATOM | 1821 | C | HIS | B | 54 | −20.721 | 39.134 | 5.941 | 1.00 | 236.78 | A | C |
| ATOM | 1822 | O | HIS | B | 54 | −20.199 | 39.104 | 7.067 | 1.00 | 221.51 | A | O |
| ATOM | 1823 | N | PRO | B | 55 | −20.247 | 39.885 | 4.912 | 1.00 | 264.73 | A | N |
| ATOM | 1824 | CA | PRO | B | 55 | −19.070 | 40.753 | 5.054 | 1.00 | 265.81 | A | C |
| ATOM | 1825 | CB | PRO | B | 55 | −18.962 | 41.428 | 3.672 | 1.00 | 267.85 | A | C |
| ATOM | 1826 | CG | PRO | B | 55 | −19.656 | 40.502 | 2.724 | 1.00 | 232.42 | A | C |
| ATOM | 1827 | CD | PRO | B | 55 | −20.778 | 39.901 | 3.530 | 1.00 | 243.78 | A | C |
| ATOM | 1828 | C | PRO | B | 55 | −19.250 | 41.790 | 6.171 | 1.00 | 249.13 | A | C |
| ATOM | 1829 | O | PRO | B | 55 | −18.588 | 42.825 | 6.160 | 1.00 | 216.75 | A | O |
| ATOM | 1830 | N | THR | B | 56 | −20.124 | 41.483 | 7.133 | 1.00 | 254.54 | A | N |
| ATOM | 1831 | CA | THR | B | 56 | −20.651 | 42.461 | 8.093 | 1.00 | 247.19 | A | C |
| ATOM | 1832 | CB | THR | B | 56 | −19.683 | 42.731 | 9.269 | 1.00 | 221.55 | A | C |
| ATOM | 1833 | OG1 | THR | B | 56 | −18.518 | 43.406 | 8.785 | 1.00 | 281.59 | A | O |
| ATOM | 1834 | CG2 | THR | B | 56 | −19.270 | 41.426 | 9.962 | 1.00 | 253.17 | A | C |
| ATOM | 1835 | C | THR | B | 56 | −21.046 | 43.762 | 7.372 | 1.00 | 268.69 | A | C |
| ATOM | 1836 | O | THR | B | 56 | −21.082 | 44.836 | 7.975 | 1.00 | 252.55 | A | O |
| ATOM | 1837 | N | ALA | B | 57 | −21.326 | 43.646 | 6.072 | 1.00 | 302.67 | A | N |
| ATOM | 1838 | CA | ALA | B | 57 | −21.876 | 44.739 | 5.266 | 1.00 | 286.91 | A | C |
| ATOM | 1839 | CB | ALA | B | 57 | −21.439 | 44.613 | 3.813 | 1.00 | 237.91 | A | C |
| ATOM | 1840 | C | ALA | B | 57 | −23.406 | 44.776 | 5.374 | 1.00 | 328.93 | A | C |
| ATOM | 1841 | O | ALA | B | 57 | −24.002 | 45.853 | 5.231 | 1.00 | 308.54 | A | O |
| ATOM | 1842 | N | PRO | B | 58 | −24.045 | 43.599 | 5.611 | 1.00 | 319.34 | A | N |
| ATOM | 1843 | CA | PRO | B | 58 | −25.481 | 43.552 | 5.895 | 1.00 | 298.35 | A | C |
| ATOM | 1844 | CB | PRO | B | 58 | −25.767 | 42.045 | 6.033 | 1.00 | 278.70 | A | C |
| ATOM | 1845 | CG | PRO | B | 58 | −24.447 | 41.419 | 6.320 | 1.00 | 286.88 | A | C |
| ATOM | 1846 | CD | PRO | B | 58 | −23.485 | 42.237 | 5.515 | 1.00 | 280.73 | A | C |
| ATOM | 1847 | C | PRO | B | 58 | −25.817 | 44.273 | 7.195 | 1.00 | 313.95 | A | C |
| ATOM | 1848 | O | PRO | B | 58 | −26.987 | 44.381 | 7.550 | 1.00 | 264.54 | A | O |
| ATOM | 1849 | N | SER | B | 74 | −13.661 | 40.968 | 13.603 | 1.00 | 188.94 | A | N |
| ATOM | 1850 | CA | SER | B | 74 | −13.701 | 40.259 | 12.310 | 1.00 | 242.83 | A | C |
| ATOM | 1851 | CB | SER | B | 74 | −12.452 | 40.582 | 11.456 | 1.00 | 183.98 | A | C |
| ATOM | 1852 | OG | SER | B | 74 | −11.816 | 41.799 | 11.842 | 1.00 | 166.01 | A | O |
| ATOM | 1853 | C | SER | B | 74 | −13.823 | 38.730 | 12.488 | 1.00 | 204.90 | A | C |
| ATOM | 1854 | O | SER | B | 74 | −14.321 | 38.238 | 13.514 | 1.00 | 194.84 | A | O |
| ATOM | 1855 | N | PHE | B | 75 | −13.359 | 37.986 | 11.484 | 1.00 | 170.96 | A | N |
| ATOM | 1856 | CA | PHE | B | 75 | −13.184 | 36.538 | 11.608 | 1.00 | 177.21 | A | C |
| ATOM | 1857 | CB | PHE | B | 75 | −13.273 | 35.831 | 10.247 | 1.00 | 164.17 | A | C |
| ATOM | 1858 | CG | PHE | B | 75 | −12.441 | 36.448 | 9.159 | 1.00 | 157.81 | A | C |
| ATOM | 1859 | CD1 | PHE | B | 75 | −11.053 | 36.374 | 9.171 | 1.00 | 173.92 | A | C |
| ATOM | 1860 | CE1 | PHE | B | 75 | −10.308 | 36.952 | 8.155 | 1.00 | 198.39 | A | C |
| ATOM | 1861 | CZ | PHE | B | 75 | −10.947 | 37.591 | 7.099 | 1.00 | 177.22 | A | C |
| ATOM | 1862 | CE2 | PHE | B | 75 | −12.329 | 37.655 | 7.060 | 1.00 | 210.37 | A | C |
| ATOM | 1863 | CD2 | PHE | B | 75 | −13.065 | 37.082 | 8.086 | 1.00 | 162.19 | A | C |
| ATOM | 1864 | C | PHE | B | 75 | −11.886 | 36.181 | 12.342 | 1.00 | 174.37 | A | C |
| ATOM | 1865 | O | PHE | B | 75 | −11.381 | 35.054 | 12.266 | 1.00 | 163.52 | A | O |
| ATOM | 1866 | N | TYR | B | 76 | −11.350 | 37.163 | 13.053 | 1.00 | 175.17 | A | N |
| ATOM | 1867 | CA | TYR | B | 76 | −10.308 | 36.949 | 14.046 | 1.00 | 175.99 | A | C |
| ATOM | 1868 | CB | TYR | B | 76 | −10.271 | 38.166 | 14.977 | 1.00 | 166.06 | A | C |
| ATOM | 1869 | CG | TYR | B | 76 | −9.481 | 37.988 | 16.246 | 1.00 | 169.26 | A | C |
| ATOM | 1870 | CD1 | TYR | B | 76 | −8.093 | 38.165 | 16.263 | 1.00 | 177.63 | A | C |
| ATOM | 1871 | CE1 | TYR | B | 76 | −7.373 | 38.012 | 17.436 | 1.00 | 206.43 | A | C |
| ATOM | 1872 | CZ | TYR | B | 76 | −8.040 | 37.688 | 18.615 | 1.00 | 194.71 | A | C |
| ATOM | 1873 | OH | TYR | B | 76 | −7.336 | 37.533 | 19.785 | 1.00 | 195.02 | A | O |
| ATOM | 1874 | CE2 | TYR | B | 76 | −9.415 | 37.514 | 18.621 | 1.00 | 200.32 | A | C |
| ATOM | 1875 | CD2 | TYR | B | 76 | −10.125 | 37.666 | 17.441 | 1.00 | 164.72 | A | C |
| ATOM | 1876 | C | TYR | B | 76 | −10.523 | 35.660 | 14.849 | 1.00 | 174.11 | A | C |
| ATOM | 1877 | O | TYR | B | 76 | −9.566 | 35.015 | 15.278 | 1.00 | 161.04 | A | O |
| ATOM | 1878 | N | GLY | B | 77 | −11.790 | 35.311 | 15.058 | 1.00 | 193.77 | A | N |
| ATOM | 1879 | CA | GLY | B | 77 | −12.155 | 34.126 | 15.836 | 1.00 | 171.65 | A | C |
| ATOM | 1880 | C | GLY | B | 77 | −11.670 | 32.817 | 15.245 | 1.00 | 161.77 | A | C |
| ATOM | 1881 | O | GLY | B | 77 | −11.006 | 32.046 | 15.934 | 1.00 | 159.35 | A | O |
| ATOM | 1882 | N | PRO | B | 78 | −12.017 | 32.546 | 13.969 | 1.00 | 162.96 | A | N |
| ATOM | 1883 | CA | PRO | B | 78 | −11.478 | 31.408 | 13.211 | 1.00 | 163.91 | A | C |
| ATOM | 1884 | CB | PRO | B | 78 | −11.951 | 31.669 | 11.784 | 1.00 | 150.87 | A | C |
| ATOM | 1885 | CG | PRO | B | 78 | −13.224 | 32.415 | 11.962 | 1.00 | 169.40 | A | C |
| ATOM | 1886 | CD | PRO | B | 78 | −13.186 | 33.133 | 13.292 | 1.00 | 164.26 | A | C |
| ATOM | 1887 | C | PRO | B | 78 | −9.967 | 31.377 | 13.233 | 1.00 | 165.94 | A | C |
| ATOM | 1888 | O | PRO | B | 78 | −9.382 | 30.296 | 13.387 | 1.00 | 171.35 | A | O |
| ATOM | 1889 | N | ALA | B | 79 | −9.348 | 32.548 | 13.083 | 1.00 | 150.62 | A | N |
| ATOM | 1890 | CA | ALA | B | 79 | −7.907 | 32.645 | 13.171 | 1.00 | 157.60 | A | C |
| ATOM | 1891 | CB | ALA | B | 79 | −7.461 | 34.092 | 13.053 | 1.00 | 188.74 | A | C |
| ATOM | 1892 | C | ALA | B | 79 | −7.448 | 32.030 | 14.487 | 1.00 | 163.56 | A | C |
| ATOM | 1893 | O | ALA | B | 79 | −6.654 | 31.089 | 14.498 | 1.00 | 178.72 | A | O |
| ATOM | 1894 | N | THR | B | 80 | −7.986 | 32.529 | 15.590 | 1.00 | 153.79 | A | N |
| ATOM | 1895 | CA | THR | B | 80 | −7.682 | 31.963 | 16.896 | 1.00 | 158.82 | A | C |
| ATOM | 1896 | CB | THR | B | 80 | −8.324 | 32.784 | 18.023 | 1.00 | 170.83 | A | C |
| ATOM | 1897 | OG1 | THR | B | 80 | −9.747 | 32.728 | 17.889 | 1.00 | 220.57 | A | O |
| ATOM | 1898 | CG2 | THR | B | 80 | −7.854 | 34.239 | 17.963 | 1.00 | 198.65 | A | C |
| ATOM | 1899 | C | THR | B | 80 | −8.152 | 30.505 | 17.002 | 1.00 | 174.86 | A | C |
| ATOM | 1900 | O | THR | B | 80 | −7.459 | 29.651 | 17.559 | 1.00 | 171.53 | A | O |
| ATOM | 1901 | N | GLY | B | 81 | −9.330 | 30.227 | 16.463 | 1.00 | 175.03 | A | N |
| ATOM | 1902 | CA | GLY | B | 81 | −9.896 | 28.881 | 16.511 | 1.00 | 189.63 | A | C |
| ATOM | 1903 | C | GLY | B | 81 | −8.966 | 27.804 | 15.974 | 1.00 | 168.57 | A | C |
| ATOM | 1904 | O | GLY | B | 81 | −8.436 | 26.970 | 16.743 | 1.00 | 153.56 | A | O |
| ATOM | 1905 | N | LEU | B | 82 | −8.749 | 27.837 | 14.657 | 1.00 | 126.77 | A | N |
| ATOM | 1906 | CA | LEU | B | 82 | −8.002 | 26.775 | 13.986 | 1.00 | 128.28 | A | C |
| ATOM | 1907 | CB | LEU | B | 82 | −8.365 | 26.681 | 12.501 | 1.00 | 122.54 | A | C |
| ATOM | 1908 | CG | LEU | B | 82 | −8.019 | 27.834 | 11.590 | 1.00 | 118.02 | A | C |
| ATOM | 1909 | CD1 | LEU | B | 82 | −6.710 | 27.531 | 10.919 | 1.00 | 138.94 | A | C |
| ATOM | 1910 | CD2 | LEU | B | 82 | −9.074 | 27.919 | 10.522 | 1.00 | 123.00 | A | C |
| ATOM | 1911 | C | LEU | B | 82 | −6.505 | 26.939 | 14.211 | 1.00 | 121.79 | A | C |
| ATOM | 1912 | O | LEU | B | 82 | −5.684 | 26.177 | 13.694 | 1.00 | 117.51 | A | O |
| ATOM | 1913 | N | LEU | B | 83 | −6.160 | 27.939 | 15.010 | 1.00 | 138.96 | A | N |
| ATOM | 1914 | CA | LEU | B | 83 | −4.801 | 28.063 | 15.504 | 1.00 | 146.25 | A | C |
| ATOM | 1915 | CB | LEU | B | 83 | −4.659 | 29.278 | 16.417 | 1.00 | 139.00 | A | C |
| ATOM | 1916 | CG | LEU | B | 83 | −3.205 | 29.554 | 16.752 | 1.00 | 153.65 | A | C |
| ATOM | 1917 | CD1 | LEU | B | 83 | −2.441 | 30.014 | 15.515 | 1.00 | 180.52 | A | C |
| ATOM | 1918 | CD2 | LEU | B | 83 | −3.123 | 30.563 | 17.868 | 1.00 | 193.10 | A | C |
| ATOM | 1919 | C | LEU | B | 83 | −4.384 | 26.784 | 16.219 | 1.00 | 144.17 | A | C |
| ATOM | 1920 | O | LEU | B | 83 | −3.443 | 26.121 | 15.797 | 1.00 | 135.91 | A | O |
| ATOM | 1921 | N | PHE | B | 84 | −5.094 | 26.427 | 17.285 | 1.00 | 141.25 | A | N |
| ATOM | 1922 | CA | PHE | B | 84 | −4.822 | 25.157 | 17.935 | 1.00 | 150.63 | A | C |
| ATOM | 1923 | CB | PHE | B | 84 | −4.885 | 25.243 | 19.462 | 1.00 | 160.57 | A | C |
| ATOM | 1924 | CG | PHE | B | 84 | −6.054 | 26.000 | 19.991 | 1.00 | 175.77 | A | C |
| ATOM | 1925 | CD1 | PHE | B | 84 | −7.247 | 25.349 | 20.269 | 1.00 | 180.76 | A | C |
| ATOM | 1926 | CE1 | PHE | B | 84 | −8.333 | 26.045 | 20.781 | 1.00 | 240.99 | A | C |
| ATOM | 1927 | CZ | PHE | B | 84 | −8.224 | 27.408 | 21.033 | 1.00 | 248.23 | A | C |
| ATOM | 1928 | CE2 | PHE | B | 84 | −7.029 | 28.069 | 20.774 | 1.00 | 226.07 | A | C |
| ATOM | 1929 | CD2 | PHE | B | 84 | −5.950 | 27.362 | 20.263 | 1.00 | 212.19 | A | C |
| ATOM | 1930 | C | PHE | B | 84 | −5.654 | 24.002 | 17.382 | 1.00 | 150.27 | A | C |
| ATOM | 1931 | O | PHE | B | 84 | −5.469 | 22.848 | 17.780 | 1.00 | 159.64 | A | O |
| ATOM | 1932 | N | GLY | B | 85 | −6.547 | 24.311 | 16.448 | 1.00 | 135.22 | A | N |
| ATOM | 1933 | CA | GLY | B | 85 | −7.091 | 23.280 | 15.581 | 1.00 | 123.60 | A | C |
| ATOM | 1934 | C | GLY | B | 85 | −5.969 | 22.504 | 14.907 | 1.00 | 129.33 | A | C |
| ATOM | 1935 | O | GLY | B | 85 | −5.937 | 21.282 | 14.968 | 1.00 | 130.71 | A | O |
| ATOM | 1936 | N | PHE | B | 86 | −5.039 | 23.222 | 14.280 | 1.00 | 131.72 | A | N |
| ATOM | 1937 | CA | PHE | B | 86 | −3.909 | 22.606 | 13.603 | 1.00 | 112.15 | A | C |
| ATOM | 1938 | CB | PHE | B | 86 | −3.092 | 23.650 | 12.867 | 1.00 | 111.21 | A | C |
| ATOM | 1939 | CG | PHE | B | 86 | −3.699 | 24.089 | 11.578 | 1.00 | 119.64 | A | C |
| ATOM | 1940 | CD1 | PHE | B | 86 | −4.631 | 23.299 | 10.934 | 1.00 | 120.00 | A | C |
| ATOM | 1941 | CE1 | PHE | B | 86 | −5.196 | 23.691 | 9.727 | 1.00 | 123.45 | A | C |
| ATOM | 1942 | CZ | PHE | B | 86 | −4.816 | 24.878 | 9.144 | 1.00 | 114.41 | A | C |
| ATOM | 1943 | CE2 | PHE | B | 86 | −3.874 | 25.677 | 9.774 | 1.00 | 162.13 | A | C |
| ATOM | 1944 | CD2 | PHE | B | 86 | −3.314 | 25.277 | 10.980 | 1.00 | 148.12 | A | C |
| ATOM | 1945 | C | PHE | B | 86 | −3.003 | 21.903 | 14.569 | 1.00 | 107.08 | A | C |
| ATOM | 1946 | O | PHE | B | 86 | −2.537 | 20.817 | 14.296 | 1.00 | 119.74 | A | O |
| ATOM | 1947 | N | THR | B | 87 | −2.759 | 22.530 | 15.705 | 1.00 | 108.43 | A | N |
| ATOM | 1948 | CA | THR | B | 87 | −1.872 | 21.964 | 16.706 | 1.00 | 127.38 | A | C |
| ATOM | 1949 | CB | THR | B | 87 | −1.948 | 22.740 | 18.029 | 1.00 | 130.30 | A | C |
| ATOM | 1950 | OG1 | THR | B | 87 | −1.518 | 24.088 | 17.810 | 1.00 | 135.69 | A | O |
| ATOM | 1951 | CG2 | THR | B | 87 | −1.070 | 22.093 | 19.081 | 1.00 | 155.05 | A | C |
| ATOM | 1952 | C | THR | B | 87 | −2.174 | 20.489 | 16.974 | 1.00 | 143.03 | A | C |
| ATOM | 1953 | O | THR | B | 87 | −1.256 | 19.686 | 17.137 | 1.00 | 154.69 | A | O |
| ATOM | 1954 | N | TYR | B | 88 | −3.459 | 20.140 | 17.016 | 1.00 | 134.04 | A | N |
| ATOM | 1955 | CA | TYR | B | 88 | −3.873 | 18.766 | 17.287 | 1.00 | 114.48 | A | C |
| ATOM | 1956 | CB | TYR | B | 88 | −5.332 | 18.729 | 17.679 | 1.00 | 93.70 | A | C |
| ATOM | 1957 | CG | TYR | B | 88 | −5.587 | 19.455 | 18.983 | 1.00 | 140.36 | A | C |
| ATOM | 1958 | CD1 | TYR | B | 88 | −4.904 | 19.095 | 20.153 | 1.00 | 163.24 | A | C |
| ATOM | 1959 | CE1 | TYR | B | 88 | −5.125 | 19.762 | 21.347 | 1.00 | 187.49 | A | C |
| ATOM | 1960 | CZ | TYR | B | 88 | −6.041 | 20.807 | 21.391 | 1.00 | 203.88 | A | C |
| ATOM | 1961 | OH | TYR | B | 88 | −6.273 | 21.473 | 22.580 | 1.00 | 230.76 | A | O |
| ATOM | 1962 | CE2 | TYR | B | 88 | −6.724 | 21.183 | 20.245 | 1.00 | 173.09 | A | C |
| ATOM | 1963 | CD2 | TYR | B | 88 | −6.496 | 20.508 | 19.055 | 1.00 | 150.46 | A | C |
| ATOM | 1964 | C | TYR | B | 88 | −3.621 | 17.891 | 16.084 | 1.00 | 115.30 | A | C |
| ATOM | 1965 | O | TYR | B | 88 | −2.990 | 16.837 | 16.180 | 1.00 | 121.77 | A | O |
| ATOM | 1966 | N | LEU | B | 89 | −4.106 | 18.351 | 14.938 | 1.00 | 109.27 | A | N |
| ATOM | 1967 | CA | LEU | B | 89 | −3.842 | 17.703 | 13.675 | 1.00 | 112.79 | A | C |
| ATOM | 1968 | CB | LEU | B | 89 | −4.536 | 18.473 | 12.563 | 1.00 | 97.99 | A | C |
| ATOM | 1969 | CG | LEU | B | 89 | −4.503 | 17.830 | 11.193 | 1.00 | 102.40 | A | C |
| ATOM | 1970 | CD1 | LEU | B | 89 | −4.849 | 16.361 | 11.276 | 1.00 | 148.73 | A | C |
| ATOM | 1971 | CD2 | LEU | B | 89 | −5.477 | 18.498 | 10.263 | 1.00 | 93.80 | A | C |
| ATOM | 1972 | C | LEU | B | 89 | −2.332 | 17.563 | 13.412 | 1.00 | 125.99 | A | C |
| ATOM | 1973 | O | LEU | B | 89 | −1.881 | 16.524 | 12.933 | 1.00 | 107.85 | A | O |
| ATOM | 1974 | N | TYR | B | 90 | −1.566 | 18.601 | 13.751 | 1.00 | 127.32 | A | N |
| ATOM | 1975 | CA | TYR | B | 90 | −0.103 | 18.553 | 13.705 | 1.00 | 130.88 | A | C |
| ATOM | 1976 | CB | TYR | B | 90 | 0.536 | 19.873 | 14.193 | 1.00 | 124.35 | A | C |
| ATOM | 1977 | CG | TYR | B | 90 | 1.785 | 20.283 | 13.409 | 1.00 | 140.81 | A | C |
| ATOM | 1978 | CD1 | TYR | B | 90 | 2.914 | 19.455 | 13.340 | 1.00 | 164.43 | A | C |
| ATOM | 1979 | CE1 | TYR | B | 90 | 4.050 | 19.822 | 12.613 | 1.00 | 158.75 | A | C |
| ATOM | 1980 | CZ | TYR | B | 90 | 4.074 | 21.021 | 11.933 | 1.00 | 153.34 | A | C |
| ATOM | 1981 | OH | TYR | B | 90 | 5.200 | 21.387 | 11.207 | 1.00 | 149.02 | A | O |
| ATOM | 1982 | CE2 | TYR | B | 90 | 2.973 | 21.860 | 11.996 | 1.00 | 145.14 | A | C |
| ATOM | 1983 | CD2 | TYR | B | 90 | 1.842 | 21.496 | 12.729 | 1.00 | 133.55 | A | C |
| ATOM | 1984 | C | TYR | B | 90 | 0.399 | 17.383 | 14.542 | 1.00 | 113.82 | A | C |
| ATOM | 1985 | O | TYR | B | 90 | 1.220 | 16.596 | 14.092 | 1.00 | 120.62 | A | O |
| ATOM | 1986 | N | ALA | B | 91 | −0.109 | 17.262 | 15.757 | 1.00 | 117.44 | A | N |
| ATOM | 1987 | CA | ALA | B | 91 | 0.282 | 16.166 | 16.627 | 1.00 | 119.04 | A | C |
| ATOM | 1988 | CB | ALA | B | 91 | −0.393 | 16.286 | 17.973 | 1.00 | 120.90 | A | C |
| ATOM | 1989 | C | ALA | B | 91 | −0.091 | 14.851 | 15.987 | 1.00 | 122.30 | A | C |
| ATOM | 1990 | O | ALA | B | 91 | 0.750 | 13.975 | 15.832 | 1.00 | 119.77 | A | O |
| ATOM | 1991 | N | ALA | B | 92 | −1.360 | 14.722 | 15.616 | 1.00 | 114.13 | A | N |
| ATOM | 1992 | CA | ALA | B | 92 | −1.865 | 13.491 | 15.060 | 1.00 | 108.53 | A | C |
| ATOM | 1993 | CB | ALA | B | 92 | −3.276 | 13.691 | 14.550 | 1.00 | 113.82 | A | C |
| ATOM | 1994 | C | ALA | B | 92 | −0.960 | 13.002 | 13.954 | 1.00 | 104.52 | A | C |
| ATOM | 1995 | O | ALA | B | 92 | −0.523 | 11.856 | 13.973 | 1.00 | 127.80 | A | O |
| ATOM | 1996 | N | ILE | B | 93 | −0.656 | 13.883 | 13.007 | 1.00 | 100.82 | A | N |
| ATOM | 1997 | CA | ILE | B | 93 | 0.147 | 13.513 | 11.842 | 1.00 | 106.34 | A | C |
| ATOM | 1998 | CB | ILE | B | 93 | 0.093 | 14.591 | 10.749 | 1.00 | 98.88 | A | C |
| ATOM | 1999 | CG1 | ILE | B | 93 | −1.266 | 14.529 | 10.072 | 1.00 | 95.46 | A | C |
| ATOM | 2000 | CD1 | ILE | B | 93 | −1.474 | 15.614 | 9.050 | 1.00 | 93.22 | A | C |
| ATOM | 2001 | CG2 | ILE | B | 93 | 1.192 | 14.400 | 9.712 | 1.00 | 91.42 | A | C |
| ATOM | 2002 | C | ILE | B | 93 | 1.581 | 13.184 | 12.208 | 1.00 | 92.96 | A | C |
| ATOM | 2003 | O | ILE | B | 93 | 2.103 | 12.164 | 11.804 | 1.00 | 98.46 | A | O |
| ATOM | 2004 | N | ASN | B | 94 | 2.205 | 14.051 | 12.986 | 1.00 | 101.17 | A | N |
| ATOM | 2005 | CA | ASN | B | 94 | 3.576 | 13.818 | 13.467 | 1.00 | 122.99 | A | C |
| ATOM | 2006 | CB | ASN | B | 94 | 4.081 | 14.981 | 14.344 | 1.00 | 157.54 | A | C |
| ATOM | 2007 | CG | ASN | B | 94 | 4.886 | 16.024 | 13.557 | 1.00 | 151.65 | A | C |
| ATOM | 2008 | OD1 | ASN | B | 94 | 5.074 | 15.913 | 12.346 | 1.00 | 165.17 | A | O |
| ATOM | 2009 | ND2 | ASN | B | 94 | 5.360 | 17.049 | 14.252 | 1.00 | 150.09 | A | N |
| ATOM | 2010 | C | ASN | B | 94 | 3.738 | 12.495 | 14.206 | 1.00 | 131.40 | A | C |
| ATOM | 2011 | O | ASN | B | 94 | 4.800 | 11.869 | 14.121 | 1.00 | 133.76 | A | O |
| ATOM | 2012 | N | HIS | B | 95 | 2.681 | 12.077 | 14.909 | 1.00 | 130.30 | A | N |
| ATOM | 2013 | CA | HIS | B | 95 | 2.670 | 10.809 | 15.649 | 1.00 | 111.63 | A | C |
| ATOM | 2014 | CB | HIS | B | 95 | 1.549 | 10.781 | 16.661 | 1.00 | 111.49 | A | C |
| ATOM | 2015 | CG | HIS | B | 95 | 1.989 | 11.178 | 18.032 | 1.00 | 141.86 | A | C |
| ATOM | 2016 | ND1 | HIS | B | 95 | 1.242 | 11.949 | 18.845 | 1.00 | 157.96 | A | N |
| ATOM | 2017 | CE1 | HIS | B | 95 | 1.905 | 12.151 | 19.994 | 1.00 | 143.77 | A | C |
| ATOM | 2018 | NE2 | HIS | B | 95 | 3.088 | 11.514 | 19.916 | 1.00 | 162.12 | A | N |
| ATOM | 2019 | CD2 | HIS | B | 95 | 3.175 | 10.910 | 18.716 | 1.00 | 161.57 | A | C |
| ATOM | 2020 | C | HIS | B | 95 | 2.535 | 9.601 | 14.780 | 1.00 | 129.14 | A | C |
| ATOM | 2021 | O | HIS | B | 95 | 3.344 | 8.669 | 14.871 | 1.00 | 138.30 | A | O |
| ATOM | 2022 | N | THR | B | 96 | 1.493 | 9.600 | 13.948 | 1.00 | 109.23 | A | N |
| ATOM | 2023 | CA | THR | B | 96 | 1.192 | 8.467 | 13.091 | 1.00 | 105.21 | A | C |
| ATOM | 2024 | CB | THR | B | 96 | −0.154 | 8.636 | 12.358 | 1.00 | 107.34 | A | C |
| ATOM | 2025 | OG1 | THR | B | 96 | −0.246 | 9.963 | 11.829 | 1.00 | 127.81 | A | O |
| ATOM | 2026 | CG2 | THR | B | 96 | −1.282 | 8.428 | 13.328 | 1.00 | 94.41 | A | C |
| ATOM | 2027 | C | THR | B | 96 | 2.319 | 8.196 | 12.101 | 1.00 | 121.65 | A | C |
| ATOM | 2028 | O | THR | B | 96 | 2.671 | 7.045 | 11.847 | 1.00 | 143.44 | A | O |
| ATOM | 2029 | N | PHE | B | 97 | 2.915 | 9.256 | 11.580 | 1.00 | 96.83 | A | N |
| ATOM | 2030 | CA | PHE | B | 97 | 3.951 | 9.106 | 10.590 | 1.00 | 106.40 | A | C |
| ATOM | 2031 | CB | PHE | B | 97 | 3.778 | 10.147 | 9.500 | 1.00 | 115.44 | A | C |
| ATOM | 2032 | CG | PHE | B | 97 | 2.574 | 9.909 | 8.644 | 1.00 | 110.63 | A | C |
| ATOM | 2033 | CD1 | PHE | B | 97 | 1.313 | 10.279 | 9.096 | 1.00 | 115.54 | A | C |
| ATOM | 2034 | CE1 | PHE | B | 97 | 0.194 | 10.045 | 8.328 | 1.00 | 128.31 | A | C |
| ATOM | 2035 | CZ | PHE | B | 97 | 0.322 | 9.450 | 7.080 | 1.00 | 130.68 | A | C |
| ATOM | 2036 | CE2 | PHE | B | 97 | 1.573 | 9.067 | 6.627 | 1.00 | 125.83 | A | C |
| ATOM | 2037 | CD2 | PHE | B | 97 | 2.697 | 9.294 | 7.400 | 1.00 | 119.74 | A | C |
| ATOM | 2038 | C | PHE | B | 97 | 5.359 | 9.111 | 11.164 | 1.00 | 128.44 | A | C |
| ATOM | 2039 | O | PHE | B | 97 | 6.324 | 8.840 | 10.451 | 1.00 | 128.02 | A | O |
| ATOM | 2040 | N | GLY | B | 98 | 5.465 | 9.417 | 12.456 | 1.00 | 127.80 | A | N |
| ATOM | 2041 | CA | GLY | B | 98 | 6.720 | 9.279 | 13.195 | 1.00 | 146.45 | A | C |
| ATOM | 2042 | C | GLY | B | 98 | 7.724 | 10.289 | 12.711 | 1.00 | 132.26 | A | C |
| ATOM | 2043 | O | GLY | B | 98 | 8.739 | 9.943 | 12.108 | 1.00 | 146.86 | A | O |
| ATOM | 2044 | N | LEU | B | 99 | 7.430 | 11.548 | 12.978 | 1.00 | 138.08 | A | N |
| ATOM | 2045 | CA | LEU | B | 99 | 8.167 | 12.631 | 12.368 | 1.00 | 140.18 | A | C |
| ATOM | 2046 | CB | LEU | B | 99 | 7.207 | 13.498 | 11.547 | 1.00 | 121.67 | A | C |
| ATOM | 2047 | CG | LEU | B | 99 | 6.413 | 12.839 | 10.427 | 1.00 | 90.18 | A | C |
| ATOM | 2048 | CD1 | LEU | B | 99 | 5.152 | 13.624 | 10.159 | 1.00 | 105.39 | A | C |
| ATOM | 2049 | CD2 | LEU | B | 99 | 7.208 | 12.786 | 9.154 | 1.00 | 98.50 | A | C |
| ATOM | 2050 | C | LEU | B | 99 | 8.935 | 13.460 | 13.419 | 1.00 | 156.14 | A | C |
| ATOM | 2051 | O | LEU | B | 99 | 8.844 | 13.208 | 14.633 | 1.00 | 157.71 | A | O |
| ATOM | 2052 | N | ASP | B | 100 | 9.694 | 14.442 | 12.934 | 1.00 | 133.86 | A | N |
| ATOM | 2053 | CA | ASP | B | 100 | 10.464 | 15.353 | 13.775 | 1.00 | 123.61 | A | C |
| ATOM | 2054 | CB | ASP | B | 100 | 11.523 | 16.067 | 12.937 | 1.00 | 148.59 | A | C |
| ATOM | 2055 | CG | ASP | B | 100 | 12.896 | 16.006 | 13.557 | 1.00 | 168.47 | A | C |
| ATOM | 2056 | OD1 | ASP | B | 100 | 13.009 | 16.178 | 14.798 | 1.00 | 155.54 | A | O |
| ATOM | 2057 | OD2 | ASP | B | 100 | 13.864 | 15.769 | 12.793 | 1.00 | 183.38 | A | O |
| ATOM | 2058 | C | ASP | B | 100 | 9.601 | 16.395 | 14.469 | 1.00 | 149.69 | A | C |
| ATOM | 2059 | O | ASP | B | 100 | 8.787 | 17.085 | 13.857 | 1.00 | 141.25 | A | O |
| ATOM | 2060 | N | TRP | B | 101 | 9.807 | 16.516 | 15.760 | 1.00 | 141.31 | A | N |
| ATOM | 2061 | CA | TRP | B | 101 | 9.014 | 17.428 | 16.564 | 1.00 | 154.35 | A | C |
| ATOM | 2062 | CB | TRP | B | 101 | 8.886 | 16.870 | 17.965 | 1.00 | 155.97 | A | C |
| ATOM | 2063 | CG | TRP | B | 101 | 8.017 | 15.652 | 18.029 | 1.00 | 205.23 | A | C |
| ATOM | 2064 | CD1 | TRP | B | 101 | 8.423 | 14.325 | 18.137 | 1.00 | 229.82 | A | C |
| ATOM | 2065 | NE1 | TRP | B | 101 | 7.339 | 13.484 | 18.138 | 1.00 | 254.34 | A | N |
| ATOM | 2066 | CE2 | TRP | B | 101 | 6.186 | 14.183 | 18.017 | 1.00 | 142.70 | A | C |
| ATOM | 2067 | CD2 | TRP | B | 101 | 6.549 | 15.600 | 17.942 | 1.00 | 187.01 | A | C |
| ATOM | 2068 | CE3 | TRP | B | 101 | 5.539 | 16.549 | 17.823 | 1.00 | 189.73 | A | C |
| ATOM | 2069 | CZ3 | TRP | B | 101 | 4.207 | 16.110 | 17.776 | 1.00 | 166.02 | A | C |
| ATOM | 2070 | CH2 | TRP | B | 101 | 3.877 | 14.747 | 17.848 | 1.00 | 137.86 | A | C |
| ATOM | 2071 | CZ2 | TRP | B | 101 | 4.860 | 13.757 | 17.969 | 1.00 | 144.63 | A | C |
| ATOM | 2072 | C | TRP | B | 101 | 9.566 | 18.827 | 16.603 | 1.00 | 156.92 | A | C |
| ATOM | 2073 | O | TRP | B | 101 | 9.117 | 19.645 | 17.395 | 1.00 | 142.72 | A | O |
| ATOM | 2074 | N | ARG | B | 102 | 10.540 | 19.121 | 15.746 | 1.00 | 167.12 | A | N |
| ATOM | 2075 | CA | ARG | B | 102 | 11.142 | 20.456 | 15.675 | 1.00 | 154.46 | A | C |
| ATOM | 2076 | CB | ARG | B | 102 | 12.474 | 20.427 | 14.892 | 1.00 | 151.67 | A | C |
| ATOM | 2077 | CG | ARG | B | 102 | 13.691 | 20.076 | 15.741 | 1.00 | 142.81 | A | C |
| ATOM | 2078 | CD | ARG | B | 102 | 14.592 | 19.037 | 15.100 | 1.00 | 152.96 | A | C |
| ATOM | 2079 | NE | ARG | B | 102 | 15.502 | 19.608 | 14.113 | 1.00 | 156.41 | A | N |
| ATOM | 2080 | CZ | ARG | B | 102 | 16.101 | 18.918 | 13.138 | 1.00 | 182.66 | A | C |
| ATOM | 2081 | NH1 | ARG | B | 102 | 15.886 | 17.618 | 12.990 | 1.00 | 174.13 | A | N |
| ATOM | 2082 | NH2 | ARG | B | 102 | 16.919 | 19.535 | 12.296 | 1.00 | 196.07 | A | N |
| ATOM | 2083 | C | ARG | B | 102 | 10.150 | 21.452 | 15.072 | 1.00 | 145.43 | A | C |
| ATOM | 2084 | O | ARG | B | 102 | 9.713 | 22.378 | 15.764 | 1.00 | 154.82 | A | O |
| ATOM | 2085 | N | PRO | B | 103 | 9.768 | 21.248 | 13.792 | 1.00 | 126.62 | A | N |
| ATOM | 2086 | CA | PRO | B | 103 | 8.818 | 22.128 | 13.139 | 1.00 | 136.26 | A | C |
| ATOM | 2087 | CB | PRO | B | 103 | 8.576 | 21.433 | 11.799 | 1.00 | 139.02 | A | C |
| ATOM | 2088 | CG | PRO | B | 103 | 9.864 | 20.752 | 11.513 | 1.00 | 147.82 | A | C |
| ATOM | 2089 | CD | PRO | B | 103 | 10.276 | 20.227 | 12.856 | 1.00 | 138.18 | A | C |
| ATOM | 2090 | C | PRO | B | 103 | 7.519 | 22.274 | 13.920 | 1.00 | 138.15 | A | C |
| ATOM | 2091 | O | PRO | B | 103 | 6.908 | 23.341 | 13.900 | 1.00 | 135.87 | A | O |
| ATOM | 2092 | N | TYR | B | 104 | 7.116 | 21.213 | 14.607 | 1.00 | 134.58 | A | N |
| ATOM | 2093 | CA | TYR | B | 104 | 6.003 | 21.303 | 15.529 | 1.00 | 135.55 | A | C |
| ATOM | 2094 | CB | TYR | B | 104 | 5.659 | 19.933 | 16.114 | 1.00 | 164.75 | A | C |
| ATOM | 2095 | CG | TYR | B | 104 | 4.554 | 19.975 | 17.166 | 1.00 | 182.60 | A | C |
| ATOM | 2096 | CD1 | TYR | B | 104 | 3.201 | 19.956 | 16.794 | 1.00 | 152.05 | A | C |
| ATOM | 2097 | CE1 | TYR | B | 104 | 2.190 | 19.997 | 17.747 | 1.00 | 127.49 | A | C |
| ATOM | 2098 | CZ | TYR | B | 104 | 2.521 | 20.038 | 19.083 | 1.00 | 163.86 | A | C |
| ATOM | 2099 | OH | TYR | B | 104 | 1.521 | 20.080 | 20.026 | 1.00 | 209.06 | A | O |
| ATOM | 2100 | CE2 | TYR | B | 104 | 3.851 | 20.054 | 19.487 | 1.00 | 193.31 | A | C |
| ATOM | 2101 | CD2 | TYR | B | 104 | 4.858 | 20.035 | 18.530 | 1.00 | 174.28 | A | C |
| ATOM | 2102 | C | TYR | B | 104 | 6.286 | 22.283 | 16.686 | 1.00 | 136.21 | A | C |
| ATOM | 2103 | O | TYR | B | 104 | 5.447 | 23.116 | 17.013 | 1.00 | 141.31 | A | O |
| ATOM | 2104 | N | SER | B | 105 | 7.458 | 22.147 | 17.306 | 1.00 | 132.20 | A | N |
| ATOM | 2105 | CA | SER | B | 105 | 7.776 | 22.873 | 18.515 | 1.00 | 148.11 | A | C |
| ATOM | 2106 | CB | SER | B | 105 | 9.011 | 22.280 | 19.195 | 1.00 | 168.86 | A | C |
| ATOM | 2107 | OG | SER | B | 105 | 10.133 | 22.264 | 18.325 | 1.00 | 190.59 | A | O |
| ATOM | 2108 | C | SER | B | 105 | 7.975 | 24.359 | 18.236 | 1.00 | 167.60 | A | C |
| ATOM | 2109 | O | SER | B | 105 | 7.564 | 25.205 | 19.036 | 1.00 | 162.84 | A | O |
| ATOM | 2110 | N | TRP | B | 106 | 8.593 | 24.668 | 17.096 | 1.00 | 156.45 | A | N |
| ATOM | 2111 | CA | TRP | B | 106 | 8.678 | 26.048 | 16.636 | 1.00 | 175.22 | A | C |
| ATOM | 2112 | CB | TRP | B | 106 | 9.418 | 26.141 | 15.301 | 1.00 | 157.69 | A | C |
| ATOM | 2113 | CG | TRP | B | 106 | 10.915 | 26.417 | 15.413 | 1.00 | 168.71 | A | C |
| ATOM | 2114 | CD1 | TRP | B | 106 | 11.945 | 25.738 | 14.764 | 1.00 | 184.37 | A | C |
| ATOM | 2115 | NE1 | TRP | B | 106 | 13.176 | 26.269 | 15.095 | 1.00 | 211.12 | A | N |
| ATOM | 2116 | CE2 | TRP | B | 106 | 13.053 | 27.303 | 15.963 | 1.00 | 259.61 | A | C |
| ATOM | 2117 | CD2 | TRP | B | 106 | 11.614 | 27.470 | 16.225 | 1.00 | 189.26 | A | C |
| ATOM | 2118 | CE3 | TRP | B | 106 | 11.210 | 28.483 | 17.098 | 1.00 | 205.29 | A | C |
| ATOM | 2119 | CZ3 | TRP | B | 106 | 12.205 | 29.303 | 17.695 | 1.00 | 233.23 | A | C |
| ATOM | 2120 | CH2 | TRP | B | 106 | 13.573 | 29.123 | 17.429 | 1.00 | 266.09 | A | C |
| ATOM | 2121 | CZ2 | TRP | B | 106 | 14.025 | 28.125 | 16.567 | 1.00 | 261.66 | A | C |
| ATOM | 2122 | C | TRP | B | 106 | 7.293 | 26.639 | 16.525 | 1.00 | 155.20 | A | C |
| ATOM | 2123 | O | TRP | B | 106 | 7.000 | 27.704 | 17.083 | 1.00 | 189.78 | A | O |
| ATOM | 2124 | N | TYR | B | 107 | 6.418 | 25.926 | 15.842 | 1.00 | 112.15 | A | N |
| ATOM | 2125 | CA | TYR | B | 107 | 5.026 | 26.331 | 15.699 | 1.00 | 130.37 | A | C |
| ATOM | 2126 | CB | TYR | B | 107 | 4.234 | 25.231 | 14.967 | 1.00 | 146.39 | A | C |
| ATOM | 2127 | CG | TYR | B | 107 | 2.747 | 25.480 | 14.838 | 1.00 | 138.22 | A | C |
| ATOM | 2128 | CD1 | TYR | B | 107 | 2.237 | 26.284 | 13.811 | 1.00 | 166.02 | A | C |
| ATOM | 2129 | CE1 | TYR | B | 107 | 0.868 | 26.506 | 13.690 | 1.00 | 154.82 | A | C |
| ATOM | 2130 | CZ | TYR | B | 107 | −0.012 | 25.915 | 14.601 | 1.00 | 141.56 | A | C |
| ATOM | 2131 | OH | TYR | B | 107 | −1.362 | 26.124 | 14.493 | 1.00 | 134.01 | A | O |
| ATOM | 2132 | CE2 | TYR | B | 107 | 0.470 | 25.109 | 15.620 | 1.00 | 127.15 | A | C |
| ATOM | 2133 | CD2 | TYR | B | 107 | 1.838 | 24.897 | 15.740 | 1.00 | 124.85 | A | C |
| ATOM | 2134 | C | TYR | B | 107 | 4.413 | 26.636 | 17.052 | 1.00 | 145.73 | A | C |
| ATOM | 2135 | O | TYR | B | 107 | 3.753 | 27.651 | 17.218 | 1.00 | 165.21 | A | O |
| ATOM | 2136 | N | SER | B | 108 | 4.656 | 25.764 | 18.021 | 1.00 | 148.03 | A | N |
| ATOM | 2137 | CA | SER | B | 108 | 4.107 | 25.917 | 19.363 | 1.00 | 173.10 | A | C |
| ATOM | 2138 | CB | SER | B | 108 | 4.529 | 24.733 | 20.227 | 1.00 | 168.82 | A | C |
| ATOM | 2139 | OG | SER | B | 108 | 3.737 | 23.583 | 19.944 | 1.00 | 200.49 | A | O |
| ATOM | 2140 | C | SER | B | 108 | 4.515 | 27.243 | 20.011 | 1.00 | 180.19 | A | C |
| ATOM | 2141 | O | SER | B | 108 | 3.711 | 27.882 | 20.699 | 1.00 | 197.45 | A | O |
| ATOM | 2142 | N | LEU | B | 109 | 5.763 | 27.657 | 19.781 | 1.00 | 173.61 | A | N |
| ATOM | 2143 | CA | LEU | B | 109 | 6.229 | 28.978 | 20.191 | 1.00 | 178.64 | A | C |
| ATOM | 2144 | CB | LEU | B | 109 | 7.722 | 29.149 | 19.899 | 1.00 | 186.35 | A | C |
| ATOM | 2145 | CG | LEU | B | 109 | 8.245 | 30.590 | 19.863 | 1.00 | 175.58 | A | C |
| ATOM | 2146 | CD1 | LEU | B | 109 | 8.690 | 31.013 | 21.250 | 1.00 | 172.66 | A | C |
| ATOM | 2147 | CD2 | LEU | B | 109 | 9.364 | 30.757 | 18.851 | 1.00 | 183.85 | A | C |
| ATOM | 2148 | C | LEU | B | 109 | 5.450 | 30.092 | 19.503 | 1.00 | 155.31 | A | C |
| ATOM | 2149 | O | LEU | B | 109 | 4.888 | 30.969 | 20.195 | 1.00 | 142.32 | A | O |
| ATOM | 2150 | N | PHE | B | 110 | 5.452 | 30.064 | 18.167 | 1.00 | 172.55 | A | N |
| ATOM | 2151 | CA | PHE | B | 110 | 4.706 | 31.014 | 17.371 | 1.00 | 215.44 | A | C |
| ATOM | 2152 | CB | PHE | B | 110 | 4.613 | 30.543 | 15.926 | 1.00 | 230.35 | A | C |
| ATOM | 2153 | CG | PHE | B | 110 | 3.618 | 31.322 | 15.110 | 1.00 | 187.93 | A | C |
| ATOM | 2154 | CD1 | PHE | B | 110 | 2.238 | 31.147 | 15.287 | 1.00 | 155.69 | A | C |
| ATOM | 2155 | CE1 | PHE | B | 110 | 1.318 | 31.886 | 14.554 | 1.00 | 139.11 | A | C |
| ATOM | 2156 | CZ | PHE | B | 110 | 1.776 | 32.811 | 13.622 | 1.00 | 184.19 | A | C |
| ATOM | 2157 | CE2 | PHE | B | 110 | 3.145 | 32.988 | 13.423 | 1.00 | 156.16 | A | C |
| ATOM | 2158 | CD2 | PHE | B | 110 | 4.054 | 32.252 | 14.178 | 1.00 | 180.31 | A | C |
| ATOM | 2159 | C | PHE | B | 110 | 3.301 | 31.194 | 17.922 | 1.00 | 184.42 | A | C |
| ATOM | 2160 | O | PHE | B | 110 | 2.816 | 32.320 | 18.042 | 1.00 | 190.68 | A | O |
| ATOM | 2161 | N | VAL | B | 111 | 2.637 | 30.082 | 18.228 | 1.00 | 179.24 | A | N |
| ATOM | 2162 | CA | VAL | B | 111 | 1.309 | 30.128 | 18.838 | 1.00 | 150.78 | A | C |
| ATOM | 2163 | CB | VAL | B | 111 | 0.727 | 28.715 | 19.096 | 1.00 | 165.70 | A | C |
| ATOM | 2164 | CG1 | VAL | B | 111 | −0.543 | 28.790 | 19.955 | 1.00 | 178.40 | A | C |
| ATOM | 2165 | CG2 | VAL | B | 111 | 0.423 | 28.024 | 17.772 | 1.00 | 134.10 | A | C |
| ATOM | 2166 | C | VAL | B | 111 | 1.325 | 30.950 | 20.117 | 1.00 | 143.53 | A | C |
| ATOM | 2167 | O | VAL | B | 111 | 0.546 | 31.907 | 20.250 | 1.00 | 174.28 | A | O |
| ATOM | 2168 | N | ALA | B | 112 | 2.222 | 30.607 | 21.042 | 1.00 | 178.72 | A | N |
| ATOM | 2169 | CA | ALA | B | 112 | 2.334 | 31.353 | 22.306 | 1.00 | 170.90 | A | C |
| ATOM | 2170 | CB | ALA | B | 112 | 3.453 | 30.782 | 23.166 | 1.00 | 179.78 | A | C |
| ATOM | 2171 | C | ALA | B | 112 | 2.551 | 32.852 | 22.069 | 1.00 | 193.06 | A | C |
| ATOM | 2172 | O | ALA | B | 112 | 1.934 | 33.693 | 22.726 | 1.00 | 194.66 | A | O |
| ATOM | 2173 | N | ILE | B | 113 | 3.402 | 33.170 | 21.092 | 1.00 | 186.05 | A | N |
| ATOM | 2174 | CA | ILE | B | 113 | 3.675 | 34.564 | 20.719 | 1.00 | 207.47 | A | C |
| ATOM | 2175 | CB | ILE | B | 113 | 4.684 | 34.666 | 19.551 | 1.00 | 171.71 | A | C |
| ATOM | 2176 | CG1 | ILE | B | 113 | 6.015 | 34.003 | 19.931 | 1.00 | 169.03 | A | C |
| ATOM | 2177 | CD1 | ILE | B | 113 | 7.046 | 34.003 | 18.828 | 1.00 | 147.99 | A | C |
| ATOM | 2178 | CG2 | ILE | B | 113 | 4.916 | 36.136 | 19.185 | 1.00 | 169.79 | A | C |
| ATOM | 2179 | C | ILE | B | 113 | 2.381 | 35.304 | 20.353 | 1.00 | 196.15 | A | C |
| ATOM | 2180 | O | ILE | B | 113 | 2.270 | 36.509 | 20.515 | 1.00 | 197.92 | A | O |
| ATOM | 2181 | N | ASN | B | 114 | 1.394 | 34.567 | 19.883 | 1.00 | 137.09 | A | N |
| ATOM | 2182 | CA | ASN | B | 114 | 0.132 | 35.166 | 19.466 | 1.00 | 167.27 | A | C |
| ATOM | 2183 | CB | ASN | B | 114 | −0.333 | 34.606 | 18.118 | 1.00 | 144.73 | A | C |
| ATOM | 2184 | CG | ASN | B | 114 | 0.621 | 34.930 | 17.002 | 1.00 | 156.45 | A | C |
| ATOM | 2185 | OD1 | ASN | B | 114 | 0.195 | 35.254 | 15.911 | 1.00 | 148.22 | A | O |
| ATOM | 2186 | ND2 | ASN | B | 114 | 1.916 | 34.841 | 17.268 | 1.00 | 166.09 | A | N |
| ATOM | 2187 | C | ASN | B | 114 | −0.952 | 35.004 | 20.512 | 1.00 | 169.04 | A | C |
| ATOM | 2188 | O | ASN | B | 114 | −2.067 | 35.484 | 20.322 | 1.00 | 213.14 | A | O |
| ATOM | 2189 | N | THR | B | 115 | −0.626 | 34.321 | 21.605 | 1.00 | 172.70 | A | N |
| ATOM | 2190 | CA | THR | B | 115 | −1.539 | 34.222 | 22.744 | 1.00 | 182.19 | A | C |
| ATOM | 2191 | CB | THR | B | 115 | −1.296 | 32.950 | 23.571 | 1.00 | 192.88 | A | C |
| ATOM | 2192 | OG1 | THR | B | 115 | −0.046 | 33.050 | 24.303 | 1.00 | 222.79 | A | O |
| ATOM | 2193 | CG2 | THR | B | 115 | −1.333 | 31.721 | 22.666 | 1.00 | 157.89 | A | C |
| ATOM | 2194 | C | THR | B | 115 | −1.412 | 35.425 | 23.665 | 1.00 | 196.78 | A | C |
| ATOM | 2195 | O | THR | B | 115 | −2.321 | 35.720 | 24.446 | 1.00 | 228.09 | A | O |
| ATOM | 2196 | N | VAL | B | 116 | −0.272 | 36.104 | 23.565 | 1.00 | 200.59 | A | N |
| ATOM | 2197 | CA | VAL | B | 116 | 0.001 | 37.328 | 24.308 | 1.00 | 211.61 | A | C |
| ATOM | 2198 | CB | VAL | B | 116 | 1.451 | 37.823 | 24.080 | 1.00 | 236.69 | A | C |
| ATOM | 2199 | CG1 | VAL | B | 116 | 1.653 | 39.176 | 24.698 | 1.00 | 253.04 | A | C |
| ATOM | 2200 | CG2 | VAL | B | 116 | 2.467 | 36.843 | 24.654 | 1.00 | 266.54 | A | C |
| ATOM | 2201 | C | VAL | B | 116 | −1.007 | 38.425 | 23.932 | 1.00 | 242.04 | A | C |
| ATOM | 2202 | O | VAL | B | 116 | −1.749 | 38.915 | 24.799 | 1.00 | 230.69 | A | O |
| ATOM | 2203 | N | PRO | B | 117 | −1.048 | 38.805 | 22.635 | 1.00 | 229.51 | A | N |
| ATOM | 2204 | CA | PRO | B | 117 | −1.988 | 39.837 | 22.186 | 1.00 | 235.64 | A | C |
| ATOM | 2205 | CB | PRO | B | 117 | −1.577 | 40.083 | 20.731 | 1.00 | 204.41 | A | C |
| ATOM | 2206 | CG | PRO | B | 117 | −0.210 | 39.502 | 20.582 | 1.00 | 166.15 | A | C |
| ATOM | 2207 | CD | PRO | B | 117 | −0.184 | 38.349 | 21.533 | 1.00 | 188.71 | A | C |
| ATOM | 2208 | C | PRO | B | 117 | −3.440 | 39.365 | 22.275 | 1.00 | 218.66 | A | C |
| ATOM | 2209 | O | PRO | B | 117 | −4.357 | 40.178 | 22.423 | 1.00 | 199.51 | A | O |
| ATOM | 2210 | N | ALA | B | 118 | −3.635 | 38.055 | 22.203 | 1.00 | 215.04 | A | N |
| ATOM | 2211 | CA | ALA | B | 118 | −4.942 | 37.458 | 22.420 | 1.00 | 203.91 | A | C |
| ATOM | 2212 | CB | ALA | B | 118 | −4.889 | 35.967 | 22.152 | 1.00 | 241.04 | A | C |
| ATOM | 2213 | C | ALA | B | 118 | −5.426 | 37.724 | 23.835 | 1.00 | 224.86 | A | C |
| ATOM | 2214 | O | ALA | B | 118 | −6.575 | 38.101 | 24.028 | 1.00 | 227.76 | A | O |
| ATOM | 2215 | N | ALA | B | 119 | −4.539 | 37.542 | 24.813 | 1.00 | 233.76 | A | N |
| ATOM | 2216 | CA | ALA | B | 119 | −4.856 | 37.803 | 26.218 | 1.00 | 240.52 | A | C |
| ATOM | 2217 | CB | ALA | B | 119 | −3.704 | 37.359 | 27.114 | 1.00 | 191.08 | A | C |
| ATOM | 2218 | C | ALA | B | 119 | −5.201 | 39.283 | 26.438 | 1.00 | 229.41 | A | C |
| ATOM | 2219 | O | ALA | B | 119 | −6.259 | 39.599 | 26.988 | 1.00 | 245.43 | A | O |
| ATOM | 2220 | N | ILE | B | 120 | −4.306 | 40.172 | 25.997 | 1.00 | 235.85 | A | N |
| ATOM | 2221 | CA | ILE | B | 120 | −4.524 | 41.625 | 26.020 | 1.00 | 237.41 | A | C |
| ATOM | 2222 | CB | ILE | B | 120 | −3.465 | 42.389 | 25.173 | 1.00 | 249.16 | A | C |
| ATOM | 2223 | CG1 | ILE | B | 120 | −2.098 | 42.403 | 25.881 | 1.00 | 250.99 | A | C |
| ATOM | 2224 | CD1 | ILE | B | 120 | −0.925 | 42.809 | 25.000 | 1.00 | 252.76 | A | C |
| ATOM | 2225 | CG2 | ILE | B | 120 | −3.934 | 43.812 | 24.853 | 1.00 | 208.57 | A | C |
| ATOM | 2226 | C | ILE | B | 120 | −5.919 | 41.964 | 25.508 | 1.00 | 221.32 | A | C |
| ATOM | 2227 | O | ILE | B | 120 | −6.709 | 42.600 | 26.209 | 1.00 | 238.08 | A | O |
| ATOM | 2228 | N | LEU | B | 121 | −6.215 | 41.525 | 24.288 | 1.00 | 200.32 | A | N |
| ATOM | 2229 | CA | LEU | B | 121 | −7.451 | 41.879 | 23.619 | 1.00 | 212.94 | A | C |
| ATOM | 2230 | CB | LEU | B | 121 | −7.352 | 41.557 | 22.118 | 1.00 | 214.78 | A | C |
| ATOM | 2231 | CG | LEU | B | 121 | −8.207 | 42.333 | 21.105 | 1.00 | 232.58 | A | C |
| ATOM | 2232 | CD1 | LEU | B | 121 | −8.518 | 43.748 | 21.585 | 1.00 | 210.76 | A | C |
| ATOM | 2233 | CD2 | LEU | B | 121 | −7.581 | 42.321 | 19.714 | 1.00 | 381.39 | A | C |
| ATOM | 2234 | C | LEU | B | 121 | −8.674 | 41.223 | 24.276 | 1.00 | 241.15 | A | C |
| ATOM | 2235 | O | LEU | B | 121 | −9.782 | 41.764 | 24.211 | 1.00 | 218.94 | A | O |
| ATOM | 2236 | N | SER | B | 122 | −8.462 | 40.083 | 24.932 | 1.00 | 246.11 | A | N |
| ATOM | 2237 | CA | SER | B | 122 | −9.564 | 39.289 | 25.493 | 1.00 | 247.59 | A | C |
| ATOM | 2238 | CB | SER | B | 122 | −9.198 | 37.801 | 25.567 | 1.00 | 274.07 | A | C |
| ATOM | 2239 | OG | SER | B | 122 | −8.064 | 37.584 | 26.393 | 1.00 | 259.38 | A | O |
| ATOM | 2240 | C | SER | B | 122 | −10.025 | 39.788 | 26.857 | 1.00 | 235.67 | A | C |
| ATOM | 2241 | O | SER | B | 122 | −11.127 | 39.466 | 27.306 | 1.00 | 229.78 | A | O |
| ATOM | 2242 | N | HIS | B | 123 | −9.181 | 40.588 | 27.500 | 1.00 | 240.95 | A | N |
| ATOM | 2243 | CA | HIS | B | 123 | −9.515 | 41.169 | 28.789 | 1.00 | 244.51 | A | C |
| ATOM | 2244 | CB | HIS | B | 123 | −8.255 | 41.496 | 29.575 | 1.00 | 280.72 | A | C |
| ATOM | 2245 | CG | HIS | B | 123 | −8.535 | 42.102 | 30.912 | 1.00 | 255.80 | A | C |
| ATOM | 2246 | ND1 | HIS | B | 123 | −9.140 | 41.411 | 31.896 | 1.00 | 216.50 | A | N |
| ATOM | 2247 | CE1 | HIS | B | 123 | −9.289 | 42.204 | 32.972 | 1.00 | 291.31 | A | C |
| ATOM | 2248 | NE2 | HIS | B | 123 | −8.776 | 43.414 | 32.669 | 1.00 | 325.21 | A | N |
| ATOM | 2249 | CD2 | HIS | B | 123 | −8.317 | 43.393 | 31.405 | 1.00 | 276.09 | A | C |
| ATOM | 2250 | C | HIS | B | 123 | −10.387 | 42.398 | 28.669 | 1.00 | 244.59 | A | C |
| ATOM | 2251 | O | HIS | B | 123 | −11.066 | 42.776 | 29.617 | 1.00 | 265.26 | A | O |
| ATOM | 2252 | N | TYR | B | 124 | −10.376 | 43.019 | 27.495 | 1.00 | 261.79 | A | N |
| ATOM | 2253 | CA | TYR | B | 124 | −11.159 | 44.227 | 27.253 | 1.00 | 274.77 | A | C |
| ATOM | 2254 | CB | TYR | B | 124 | −10.342 | 45.257 | 26.457 | 1.00 | 266.53 | A | C |
| ATOM | 2255 | CG | TYR | B | 124 | −8.995 | 45.624 | 27.047 | 1.00 | 292.19 | A | C |
| ATOM | 2256 | CD1 | TYR | B | 124 | −8.769 | 45.567 | 28.433 | 1.00 | 271.11 | A | C |
| ATOM | 2257 | CE1 | TYR | B | 124 | −7.538 | 45.932 | 28.973 | 1.00 | 287.46 | A | C |
| ATOM | 2258 | CZ | TYR | B | 124 | −6.504 | 46.338 | 28.109 | 1.00 | 303.94 | A | C |
| ATOM | 2259 | OH | TYR | B | 124 | −5.256 | 46.710 | 28.590 | 1.00 | 277.59 | A | O |
| ATOM | 2260 | CE2 | TYR | B | 124 | −6.719 | 46.418 | 26.744 | 1.00 | 293.35 | A | C |
| ATOM | 2261 | CD2 | TYR | B | 124 | −7.954 | 46.066 | 26.219 | 1.00 | 293.03 | A | C |
| ATOM | 2262 | C | TYR | B | 124 | −12.470 | 43.913 | 26.523 | 1.00 | 242.15 | A | C |
| ATOM | 2263 | O | TYR | B | 124 | −12.876 | 44.638 | 25.615 | 1.00 | 292.69 | A | O |
| ATOM | 2264 | N | SER | B | 125 | −13.117 | 42.819 | 26.910 | 1.00 | 229.01 | A | N |
| ATOM | 2265 | CA | SER | B | 125 | −14.382 | 42.408 | 26.306 | 1.00 | 254.45 | A | C |
| ATOM | 2266 | CB | SER | B | 125 | −14.141 | 41.345 | 25.221 | 1.00 | 240.40 | A | C |
| ATOM | 2267 | OG | SER | B | 125 | −13.560 | 41.888 | 24.049 | 1.00 | 236.80 | A | O |
| ATOM | 2268 | C | SER | B | 125 | −15.323 | 41.833 | 27.359 | 1.00 | 288.51 | A | C |
| ATOM | 2269 | O | SER | B | 125 | −14.898 | 41.497 | 28.467 | 1.00 | 285.49 | A | O |
| ATOM | 2270 | N | ASP | B | 126 | −16.606 | 41.738 | 27.006 | 1.00 | 274.24 | A | N |
| ATOM | 2271 | CA | ASP | B | 126 | −17.537 | 40.823 | 27.661 | 1.00 | 266.05 | A | C |
| ATOM | 2272 | CB | ASP | B | 126 | −17.204 | 39.368 | 27.253 | 1.00 | 256.49 | A | C |
| ATOM | 2273 | CG | ASP | B | 126 | −17.229 | 39.128 | 25.749 | 1.00 | 274.01 | A | C |
| ATOM | 2274 | OD1 | ASP | B | 126 | −17.820 | 39.949 | 25.027 | 1.00 | 288.20 | A | O |
| ATOM | 2275 | OD2 | ASP | B | 126 | −16.660 | 38.102 | 25.302 | 1.00 | 287.96 | A | O |
| ATOM | 2276 | C | ASP | B | 126 | −17.566 | 40.897 | 29.195 | 1.00 | 253.77 | A | C |
| ATOM | 2277 | O | ASP | B | 126 | −17.488 | 39.865 | 29.867 | 1.00 | 280.82 | A | O |
| ATOM | 2278 | N | MET | B | 127 | −17.660 | 42.099 | 29.756 | 1.00 | 275.99 | A | N |
| ATOM | 2279 | CA | MET | B | 127 | −17.916 | 42.240 | 31.200 | 1.00 | 274.20 | A | C |
| ATOM | 2280 | CB | MET | B | 127 | −17.452 | 43.603 | 31.710 | 1.00 | 252.43 | A | C |
| ATOM | 2281 | CG | MET | B | 127 | −15.960 | 43.827 | 31.532 | 1.00 | 255.62 | A | C |
| ATOM | 2282 | SD | MET | B | 127 | −15.560 | 45.532 | 31.114 | 1.00 | 286.52 | A | S |
| ATOM | 2283 | CE | MET | B | 127 | −14.150 | 45.303 | 30.042 | 1.00 | 292.24 | A | C |
| ATOM | 2284 | C | MET | B | 127 | −19.410 | 42.018 | 31.439 | 1.00 | 285.56 | A | C |
| ATOM | 2285 | O | MET | B | 127 | −20.239 | 42.881 | 31.125 | 1.00 | 274.50 | A | O |
| ATOM | 2286 | N | LEU | B | 128 | −19.745 | 40.857 | 31.999 | 1.00 | 294.34 | A | N |
| ATOM | 2287 | CA | LEU | B | 128 | −21.100 | 40.316 | 31.882 | 1.00 | 278.10 | A | C |
| ATOM | 2288 | CB | LEU | B | 128 | −21.054 | 38.850 | 31.440 | 1.00 | 259.87 | A | C |
| ATOM | 2289 | CG | LEU | B | 128 | −20.358 | 38.598 | 30.099 | 1.00 | 214.18 | A | C |
| ATOM | 2290 | CD1 | LEU | B | 128 | −20.316 | 37.116 | 29.766 | 1.00 | 179.75 | A | C |
| ATOM | 2291 | CD2 | LEU | B | 128 | −20.999 | 39.403 | 28.974 | 1.00 | 195.37 | A | C |
| ATOM | 2292 | C | LEU | B | 128 | −21.966 | 40.500 | 33.118 | 1.00 | 281.28 | A | C |
| ATOM | 2293 | O | LEU | B | 128 | −23.153 | 40.831 | 33.002 | 1.00 | 264.84 | A | O |
| ATOM | 2294 | N | ASP | B | 129 | −21.372 | 40.302 | 34.294 | 1.00 | 307.68 | A | N |
| ATOM | 2295 | CA | ASP | B | 129 | −22.025 | 40.630 | 35.567 | 1.00 | 297.57 | A | C |
| ATOM | 2296 | CB | ASP | B | 129 | −22.721 | 42.015 | 35.511 | 1.00 | 311.55 | A | C |
| ATOM | 2297 | CG | ASP | B | 129 | −21.755 | 43.186 | 35.246 | 1.00 | 311.28 | A | C |
| ATOM | 2298 | OD1 | ASP | B | 129 | −20.529 | 43.032 | 35.434 | 1.00 | 354.29 | A | O |
| ATOM | 2299 | OD2 | ASP | B | 129 | −22.237 | 44.281 | 34.856 | 1.00 | 288.16 | A | O |
| ATOM | 2300 | C | ASP | B | 129 | −23.059 | 39.577 | 35.990 | 1.00 | 299.19 | A | C |
| ATOM | 2301 | O | ASP | B | 129 | −23.663 | 39.699 | 37.055 | 1.00 | 309.95 | A | O |
| ATOM | 2302 | N | ASP | B | 130 | −23.257 | 38.546 | 35.173 | 1.00 | 274.77 | A | N |
| ATOM | 2303 | CA | ASP | B | 130 | −24.448 | 37.703 | 35.279 | 1.00 | 263.64 | A | C |
| ATOM | 2304 | CB | ASP | B | 130 | −25.237 | 37.773 | 33.976 | 1.00 | 281.01 | A | C |
| ATOM | 2305 | CG | ASP | B | 130 | −25.993 | 39.091 | 33.830 | 1.00 | 301.15 | A | C |
| ATOM | 2306 | OD1 | ASP | B | 130 | −26.050 | 39.877 | 34.796 | 1.00 | 295.84 | A | O |
| ATOM | 2307 | OD2 | ASP | B | 130 | −26.542 | 39.362 | 32.751 | 1.00 | 289.33 | A | O |
| ATOM | 2308 | C | ASP | B | 130 | −24.195 | 36.260 | 35.649 | 1.00 | 275.05 | A | C |
| ATOM | 2309 | O | ASP | B | 130 | −25.071 | 35.413 | 35.509 | 1.00 | 284.18 | A | O |
| ATOM | 2310 | N | HIS | B | 131 | −23.009 | 35.988 | 36.165 | 1.00 | 288.47 | A | N |
| ATOM | 2311 | CA | HIS | B | 131 | −22.554 | 34.623 | 36.353 | 1.00 | 301.92 | A | C |
| ATOM | 2312 | CB | HIS | B | 131 | −21.114 | 34.499 | 35.885 | 1.00 | 290.05 | A | C |
| ATOM | 2313 | CG | HIS | B | 131 | −20.909 | 34.918 | 34.457 | 1.00 | 263.21 | A | C |
| ATOM | 2314 | ND1 | HIS | B | 131 | −21.467 | 36.036 | 33.937 | 1.00 | 221.38 | A | N |
| ATOM | 2315 | CE1 | HIS | B | 131 | −21.114 | 36.140 | 32.654 | 1.00 | 213.03 | A | C |
| ATOM | 2316 | NE2 | HIS | B | 131 | −20.331 | 35.099 | 32.349 | 1.00 | 244.86 | A | N |
| ATOM | 2317 | CD2 | HIS | B | 131 | −20.186 | 34.329 | 33.440 | 1.00 | 285.51 | A | C |
| ATOM | 2318 | C | HIS | B | 131 | −22.668 | 34.152 | 37.768 | 1.00 | 331.51 | A | C |
| ATOM | 2319 | O | HIS | B | 131 | −22.521 | 34.937 | 38.710 | 1.00 | 314.05 | A | O |
| ATOM | 2320 | N | LYS | B | 132 | −22.941 | 32.859 | 37.929 | 1.00 | 364.89 | A | N |
| ATOM | 2321 | CA | LYS | B | 132 | −22.710 | 32.183 | 39.197 | 1.00 | 348.67 | A | C |
| ATOM | 2322 | CB | LYS | B | 132 | −23.646 | 30.972 | 39.408 | 1.00 | 272.16 | A | C |
| ATOM | 2323 | CG | LYS | B | 132 | −24.893 | 31.310 | 40.240 | 1.00 | 221.74 | A | C |
| ATOM | 2324 | CD | LYS | B | 132 | −25.829 | 30.115 | 40.440 | 1.00 | 301.44 | A | C |
| ATOM | 2325 | CE | LYS | B | 132 | −26.382 | 29.988 | 41.870 | 1.00 | 280.31 | A | C |
| ATOM | 2326 | NZ | LYS | B | 132 | −27.127 | 31.157 | 42.491 | 1.00 | 250.59 | A | N |
| ATOM | 2327 | C | LYS | B | 132 | −21.211 | 31.844 | 39.315 | 1.00 | 362.60 | A | C |
| ATOM | 2328 | O | LYS | B | 132 | −20.773 | 31.283 | 40.323 | 1.00 | 373.32 | A | O |
| ATOM | 2329 | N | VAL | B | 133 | −20.427 | 32.218 | 38.294 | 1.00 | 348.65 | A | N |
| ATOM | 2330 | CA | VAL | B | 133 | −18.956 | 32.240 | 38.399 | 1.00 | 324.89 | A | C |
| ATOM | 2331 | CB | VAL | B | 133 | −18.277 | 32.852 | 37.137 | 1.00 | 331.87 | A | C |
| ATOM | 2332 | CG1 | VAL | B | 133 | −18.022 | 34.346 | 37.294 | 1.00 | 279.74 | A | C |
| ATOM | 2333 | CG2 | VAL | B | 133 | −16.983 | 32.115 | 36.797 | 1.00 | 252.08 | A | C |
| ATOM | 2334 | C | VAL | B | 133 | −18.569 | 32.949 | 39.715 | 1.00 | 339.68 | A | C |
| ATOM | 2335 | O | VAL | B | 133 | −19.325 | 33.775 | 40.228 | 1.00 | 394.78 | A | O |
| ATOM | 2336 | N | LEU | B | 134 | −17.402 | 32.616 | 40.255 | 1.00 | 279.69 | A | N |
| ATOM | 2337 | CA | LEU | B | 134 | −17.132 | 32.781 | 41.707 | 1.00 | 318.71 | A | C |
| ATOM | 2338 | CB | LEU | B | 134 | −15.878 | 32.000 | 42.112 | 1.00 | 323.28 | A | C |
| ATOM | 2339 | CG | LEU | B | 134 | −15.885 | 30.474 | 41.949 | 1.00 | 316.37 | A | C |
| ATOM | 2340 | CD1 | LEU | B | 134 | −16.062 | 30.036 | 40.497 | 1.00 | 359.02 | A | C |
| ATOM | 2341 | CD2 | LEU | B | 134 | −14.597 | 29.871 | 42.510 | 1.00 | 252.29 | A | C |
| ATOM | 2342 | C | LEU | B | 134 | −17.071 | 34.208 | 42.264 | 1.00 | 356.31 | A | C |
| ATOM | 2343 | O | LEU | B | 134 | −17.042 | 35.175 | 41.501 | 1.00 | 334.19 | A | O |
| ATOM | 2344 | N | GLY | B | 135 | −17.060 | 34.322 | 43.604 | 1.00 | 384.63 | A | N |
| ATOM | 2345 | CA | GLY | B | 135 | −17.180 | 35.595 | 44.341 | 1.00 | 340.50 | A | C |
| ATOM | 2346 | C | GLY | B | 135 | −15.927 | 36.454 | 44.474 | 1.00 | 326.07 | A | C |
| ATOM | 2347 | O | GLY | B | 135 | −15.963 | 37.545 | 45.061 | 1.00 | 326.19 | A | O |
| ATOM | 2348 | N | ILE | B | 136 | −14.808 | 35.952 | 43.955 | 1.00 | 372.30 | A | N |
| ATOM | 2349 | CA | ILE | B | 136 | −13.605 | 36.771 | 43.823 | 1.00 | 357.96 | A | C |
| ATOM | 2350 | CB | ILE | B | 136 | −12.302 | 35.989 | 44.121 | 1.00 | 284.37 | A | C |
| ATOM | 2351 | CG1 | ILE | B | 136 | −12.474 | 35.022 | 45.300 | 1.00 | 245.69 | A | C |
| ATOM | 2352 | CD1 | ILE | B | 136 | −11.567 | 33.812 | 45.232 | 1.00 | 247.11 | A | C |
| ATOM | 2353 | CG2 | ILE | B | 136 | −11.173 | 36.968 | 44.414 | 1.00 | 263.80 | A | C |
| ATOM | 2354 | C | ILE | B | 136 | −13.550 | 37.379 | 42.407 | 1.00 | 372.18 | A | C |
| ATOM | 2355 | O | ILE | B | 136 | −13.026 | 36.769 | 41.474 | 1.00 | 390.97 | A | O |
| ATOM | 2356 | N | THR | B | 137 | −14.107 | 38.583 | 42.257 | 1.00 | 384.70 | A | N |
| ATOM | 2357 | CA | THR | B | 137 | −14.128 | 39.316 | 40.965 | 1.00 | 347.95 | A | C |
| ATOM | 2358 | CB | THR | B | 137 | −12.853 | 40.174 | 40.772 | 1.00 | 293.23 | A | C |
| ATOM | 2359 | OG1 | THR | B | 137 | −11.684 | 39.356 | 40.954 | 1.00 | 333.20 | A | O |
| ATOM | 2360 | CG2 | THR | B | 137 | −12.844 | 41.395 | 41.775 | 1.00 | 197.98 | A | C |
| ATOM | 2361 | C | THR | B | 137 | −14.448 | 38.449 | 39.718 | 1.00 | 310.82 | A | C |
| ATOM | 2362 | O | THR | B | 137 | −15.562 | 37.947 | 39.592 | 1.00 | 304.15 | A | O |
| ATOM | 2363 | N | GLU | B | 138 | −13.486 | 38.266 | 38.815 | 1.00 | 300.61 | A | N |
| ATOM | 2364 | CA | GLU | B | 138 | −13.664 | 37.409 | 37.617 | 1.00 | 285.55 | A | C |
| ATOM | 2365 | CB | GLU | B | 138 | −14.293 | 36.062 | 37.975 | 1.00 | 295.84 | A | C |
| ATOM | 2366 | CG | GLU | B | 138 | −13.412 | 35.181 | 38.833 | 1.00 | 277.39 | A | C |
| ATOM | 2367 | CD | GLU | B | 138 | −14.219 | 34.211 | 39.669 | 1.00 | 304.88 | A | C |
| ATOM | 2368 | OE1 | GLU | B | 138 | −14.883 | 33.335 | 39.075 | 1.00 | 314.48 | A | O |
| ATOM | 2369 | OE2 | GLU | B | 138 | −14.189 | 34.336 | 40.921 | 1.00 | 315.20 | A | O |
| ATOM | 2370 | C | GLU | B | 138 | −14.450 | 38.058 | 36.474 | 1.00 | 263.66 | A | C |
| ATOM | 2371 | O | GLU | B | 138 | −15.250 | 37.403 | 35.790 | 1.00 | 221.49 | A | O |
| ATOM | 2372 | N | GLY | B | 139 | −14.226 | 39.351 | 36.287 | 1.00 | 246.66 | A | N |
| ATOM | 2373 | CA | GLY | B | 139 | −14.704 | 40.025 | 35.093 | 1.00 | 197.01 | A | C |
| ATOM | 2374 | C | GLY | B | 139 | −13.894 | 39.530 | 33.932 | 1.00 | 234.18 | A | C |
| ATOM | 2375 | O | GLY | B | 139 | −12.761 | 39.966 | 33.725 | 1.00 | 226.71 | A | O |
| ATOM | 2376 | N | ASP | B | 140 | −14.475 | 38.582 | 33.206 | 1.00 | 257.05 | A | N |
| ATOM | 2377 | CA | ASP | B | 140 | −13.888 | 38.027 | 31.973 | 1.00 | 245.63 | A | C |
| ATOM | 2378 | CB | ASP | B | 140 | −14.115 | 38.977 | 30.800 | 1.00 | 249.75 | A | C |
| ATOM | 2379 | CG | ASP | B | 140 | −13.757 | 38.349 | 29.468 | 1.00 | 275.77 | A | C |
| ATOM | 2380 | OD1 | ASP | B | 140 | −14.423 | 37.366 | 29.072 | 1.00 | 281.67 | A | O |
| ATOM | 2381 | OD2 | ASP | B | 140 | −12.806 | 38.838 | 28.823 | 1.00 | 251.05 | A | O |
| ATOM | 2382 | C | ASP | B | 140 | −12.405 | 37.628 | 32.045 | 1.00 | 269.23 | A | C |
| ATOM | 2383 | O | ASP | B | 140 | −11.545 | 38.305 | 31.473 | 1.00 | 203.75 | A | O |
| ATOM | 2384 | N | TRP | B | 141 | −12.122 | 36.516 | 32.720 | 1.00 | 285.44 | A | N |
| ATOM | 2385 | CA | TRP | B | 141 | −10.747 | 35.999 | 32.853 | 1.00 | 265.09 | A | C |
| ATOM | 2386 | CB | TRP | B | 141 | −10.532 | 35.306 | 34.200 | 1.00 | 267.67 | A | C |
| ATOM | 2387 | CG | TRP | B | 141 | −11.602 | 34.322 | 34.611 | 1.00 | 292.06 | A | C |
| ATOM | 2388 | CD1 | TRP | B | 141 | −12.968 | 34.560 | 34.759 | 1.00 | 286.55 | A | C |
| ATOM | 2389 | NE1 | TRP | B | 141 | −13.627 | 33.433 | 35.184 | 1.00 | 292.45 | A | N |
| ATOM | 2390 | CE2 | TRP | B | 141 | −12.761 | 32.405 | 35.348 | 1.00 | 322.95 | A | C |
| ATOM | 2391 | CD2 | TRP | B | 141 | −11.421 | 32.913 | 35.007 | 1.00 | 314.36 | A | C |
| ATOM | 2392 | CE3 | TRP | B | 141 | −10.328 | 32.049 | 35.101 | 1.00 | 324.22 | A | C |
| ATOM | 2393 | CZ3 | TRP | B | 141 | −10.555 | 30.726 | 35.518 | 1.00 | 315.74 | A | C |
| ATOM | 2394 | CH2 | TRP | B | 141 | −11.844 | 30.261 | 35.840 | 1.00 | 284.29 | A | C |
| ATOM | 2395 | CZ2 | TRP | B | 141 | −12.971 | 31.090 | 35.760 | 1.00 | 283.00 | A | C |
| ATOM | 2396 | C | TRP | B | 141 | −10.266 | 35.139 | 31.706 | 1.00 | 276.63 | A | C |
| ATOM | 2397 | O | TRP | B | 141 | −9.234 | 34.455 | 31.815 | 1.00 | 252.64 | A | O |
| ATOM | 2398 | N | TRP | B | 142 | −11.003 | 35.181 | 30.590 | 1.00 | 256.48 | A | N |
| ATOM | 2399 | CA | TRP | B | 142 | −10.498 | 34.718 | 29.293 | 1.00 | 243.83 | A | C |
| ATOM | 2400 | CB | TRP | B | 142 | −11.333 | 35.302 | 28.149 | 1.00 | 268.11 | A | C |
| ATOM | 2401 | CG | TRP | B | 142 | −12.341 | 34.342 | 27.555 | 1.00 | 271.87 | A | C |
| ATOM | 2402 | CD1 | TRP | B | 142 | −13.249 | 33.529 | 28.238 | 1.00 | 283.02 | A | C |
| ATOM | 2403 | NE1 | TRP | B | 142 | −14.005 | 32.790 | 27.355 | 1.00 | 273.84 | A | N |
| ATOM | 2404 | CE2 | TRP | B | 142 | −13.662 | 33.076 | 26.080 | 1.00 | 224.72 | A | C |
| ATOM | 2405 | CD2 | TRP | B | 142 | −12.596 | 34.082 | 26.126 | 1.00 | 258.31 | A | C |
| ATOM | 2406 | CE3 | TRP | B | 142 | −12.058 | 34.556 | 24.923 | 1.00 | 240.91 | A | C |
| ATOM | 2407 | CZ3 | TRP | B | 142 | −12.564 | 34.050 | 23.712 | 1.00 | 241.16 | A | C |
| ATOM | 2408 | CH2 | TRP | B | 142 | −13.581 | 33.083 | 23.691 | 1.00 | 223.79 | A | C |
| ATOM | 2409 | CZ2 | TRP | B | 142 | −14.148 | 32.581 | 24.880 | 1.00 | 211.34 | A | C |
| ATOM | 2410 | C | TRP | B | 142 | −9.061 | 35.105 | 29.092 | 1.00 | 250.17 | A | C |
| ATOM | 2411 | O | TRP | B | 142 | −8.286 | 34.353 | 28.501 | 1.00 | 256.13 | A | O |
| ATOM | 2412 | N | ALA | B | 143 | −8.702 | 36.289 | 29.584 | 1.00 | 224.19 | A | N |
| ATOM | 2413 | CA | ALA | B | 143 | −7.347 | 36.812 | 29.451 | 1.00 | 243.65 | A | C |
| ATOM | 2414 | CB | ALA | B | 143 | −7.205 | 38.119 | 30.212 | 1.00 | 250.55 | A | C |
| ATOM | 2415 | C | ALA | B | 143 | −6.289 | 35.811 | 29.914 | 1.00 | 235.24 | A | C |
| ATOM | 2416 | O | ALA | B | 143 | −5.337 | 35.529 | 29.176 | 1.00 | 240.29 | A | O |
| ATOM | 2417 | N | ILE | B | 144 | −6.483 | 35.252 | 31.109 | 1.00 | 236.60 | A | N |
| ATOM | 2418 | CA | ILE | B | 144 | −5.465 | 34.428 | 31.767 | 1.00 | 225.93 | A | C |
| ATOM | 2419 | CB | ILE | B | 144 | −5.651 | 34.408 | 33.303 | 1.00 | 239.84 | A | C |
| ATOM | 2420 | CG1 | ILE | B | 144 | −6.960 | 33.700 | 33.692 | 1.00 | 251.37 | A | C |
| ATOM | 2421 | CD1 | ILE | B | 144 | −7.376 | 33.878 | 35.132 | 1.00 | 253.41 | A | C |
| ATOM | 2422 | CG2 | ILE | B | 144 | −5.590 | 35.823 | 33.870 | 1.00 | 210.81 | A | C |
| ATOM | 2423 | C | ILE | B | 144 | −5.456 | 33.006 | 31.226 | 1.00 | 209.16 | A | C |
| ATOM | 2424 | O | ILE | B | 144 | −4.489 | 32.266 | 31.386 | 1.00 | 215.70 | A | O |
| ATOM | 2425 | N | ILE | B | 145 | −6.556 | 32.641 | 30.593 | 1.00 | 189.89 | A | N |
| ATOM | 2426 | CA | ILE | B | 145 | −6.621 | 31.398 | 29.803 | 1.00 | 179.18 | A | C |
| ATOM | 2427 | CB | ILE | B | 145 | −8.038 | 31.170 | 29.250 | 1.00 | 191.60 | A | C |
| ATOM | 2428 | CG1 | ILE | B | 145 | −8.974 | 30.717 | 30.372 | 1.00 | 212.85 | A | C |
| ATOM | 2429 | CD1 | ILE | B | 145 | −10.431 | 30.906 | 30.028 | 1.00 | 232.08 | A | C |
| ATOM | 2430 | CG2 | ILE | B | 145 | −8.022 | 30.171 | 28.115 | 1.00 | 189.62 | A | C |
| ATOM | 2431 | C | ILE | B | 145 | −5.608 | 31.434 | 28.673 | 1.00 | 213.96 | A | C |
| ATOM | 2432 | O | ILE | B | 145 | −4.802 | 30.503 | 28.544 | 1.00 | 200.53 | A | O |
| ATOM | 2433 | N | TRP | B | 146 | −5.658 | 32.496 | 27.855 | 1.00 | 216.31 | A | N |
| ATOM | 2434 | CA | TRP | B | 146 | −4.793 | 32.616 | 26.661 | 1.00 | 257.32 | A | C |
| ATOM | 2435 | CB | TRP | B | 146 | −5.034 | 33.944 | 25.957 | 1.00 | 268.05 | A | C |
| ATOM | 2436 | CG | TRP | B | 146 | −6.188 | 33.931 | 24.987 | 1.00 | 271.72 | A | C |
| ATOM | 2437 | CD1 | TRP | B | 146 | −7.421 | 34.564 | 25.122 | 1.00 | 274.77 | A | C |
| ATOM | 2438 | NE1 | TRP | B | 146 | −8.218 | 34.332 | 24.026 | 1.00 | 278.76 | A | N |
| ATOM | 2439 | CE2 | TRP | B | 146 | −7.579 | 33.555 | 23.127 | 1.00 | 266.19 | A | C |
| ATOM | 2440 | CD2 | TRP | B | 146 | −6.251 | 33.252 | 23.685 | 1.00 | 289.72 | A | C |
| ATOM | 2441 | CE3 | TRP | B | 146 | −5.372 | 32.454 | 22.948 | 1.00 | 252.55 | A | C |
| ATOM | 2442 | CZ3 | TRP | B | 146 | −5.797 | 31.969 | 21.700 | 1.00 | 251.80 | A | C |
| ATOM | 2443 | CH2 | TRP | B | 146 | −7.068 | 32.271 | 21.190 | 1.00 | 297.79 | A | C |
| ATOM | 2444 | CZ2 | TRP | B | 146 | −7.983 | 33.071 | 21.892 | 1.00 | 309.49 | A | C |
| ATOM | 2445 | C | TRP | B | 146 | −3.337 | 32.499 | 27.008 | 1.00 | 248.78 | A | C |
| ATOM | 2446 | O | TRP | B | 146 | −2.543 | 31.935 | 26.249 | 1.00 | 248.78 | A | O |
| ATOM | 2447 | N | LEU | B | 147 | −2.979 | 33.048 | 28.164 | 1.00 | 270.61 | A | N |
| ATOM | 2448 | CA | LEU | B | 147 | −1.622 | 32.967 | 28.674 | 1.00 | 276.81 | A | C |
| ATOM | 2449 | CB | LEU | B | 147 | −1.385 | 34.031 | 29.744 | 1.00 | 251.57 | A | C |
| ATOM | 2450 | CG | LEU | B | 147 | −1.555 | 35.467 | 29.253 | 1.00 | 251.86 | A | C |
| ATOM | 2451 | CD1 | LEU | B | 147 | −1.435 | 36.396 | 30.452 | 1.00 | 257.66 | A | C |
| ATOM | 2452 | CD2 | LEU | B | 147 | −0.568 | 35.838 | 28.143 | 1.00 | 245.57 | A | C |
| ATOM | 2453 | C | LEU | B | 147 | −1.335 | 31.578 | 29.219 | 1.00 | 272.01 | A | C |
| ATOM | 2454 | O | LEU | B | 147 | −0.198 | 31.112 | 29.146 | 1.00 | 292.14 | A | O |
| ATOM | 2455 | N | ALA | B | 148 | −2.363 | 30.921 | 29.753 | 1.00 | 245.43 | A | N |
| ATOM | 2456 | CA | ALA | B | 148 | −2.235 | 29.544 | 30.236 | 1.00 | 225.20 | A | C |
| ATOM | 2457 | CB | ALA | B | 148 | −3.451 | 29.162 | 31.073 | 1.00 | 237.67 | A | C |
| ATOM | 2458 | C | ALA | B | 148 | −2.016 | 28.545 | 29.080 | 1.00 | 248.96 | A | C |
| ATOM | 2459 | O | ALA | B | 148 | −1.172 | 27.652 | 29.157 | 1.00 | 239.99 | A | O |
| ATOM | 2460 | N | TRP | B | 149 | −2.790 | 28.729 | 28.015 | 1.00 | 260.08 | A | N |
| ATOM | 2461 | CA | TRP | B | 149 | −2.658 | 27.943 | 26.808 | 1.00 | 240.46 | A | C |
| ATOM | 2462 | CB | TRP | B | 149 | −3.832 | 28.192 | 25.870 | 1.00 | 268.14 | A | C |
| ATOM | 2463 | CG | TRP | B | 149 | −5.137 | 27.581 | 26.344 | 1.00 | 252.97 | A | C |
| ATOM | 2464 | CD1 | TRP | B | 149 | −5.323 | 26.642 | 27.356 | 1.00 | 252.49 | A | C |
| ATOM | 2465 | NE1 | TRP | B | 149 | −6.641 | 26.309 | 27.489 | 1.00 | 245.65 | A | N |
| ATOM | 2466 | CE2 | TRP | B | 149 | −7.394 | 26.975 | 26.592 | 1.00 | 232.10 | A | C |
| ATOM | 2467 | CD2 | TRP | B | 149 | −6.483 | 27.821 | 25.807 | 1.00 | 246.06 | A | C |
| ATOM | 2468 | CE3 | TRP | B | 149 | −7.006 | 28.620 | 24.797 | 1.00 | 250.62 | A | C |
| ATOM | 2469 | CZ3 | TRP | B | 149 | −8.386 | 28.593 | 24.576 | 1.00 | 255.97 | A | C |
| ATOM | 2470 | CH2 | TRP | B | 149 | −9.241 | 27.776 | 25.337 | 1.00 | 227.19 | A | C |
| ATOM | 2471 | CZ2 | TRP | B | 149 | −8.761 | 26.955 | 26.362 | 1.00 | 224.29 | A | C |
| ATOM | 2472 | C | TRP | B | 149 | −1.364 | 28.221 | 26.123 | 1.00 | 275.50 | A | C |
| ATOM | 2473 | O | TRP | B | 149 | −0.679 | 27.303 | 25.707 | 1.00 | 263.97 | A | O |
| ATOM | 2474 | N | GLY | B | 150 | −1.014 | 29.505 | 26.015 | 1.00 | 246.19 | A | N |
| ATOM | 2475 | CA | GLY | B | 150 | 0.299 | 29.896 | 25.509 | 1.00 | 262.84 | A | C |
| ATOM | 2476 | C | GLY | B | 150 | 1.410 | 29.207 | 26.270 | 1.00 | 280.00 | A | C |
| ATOM | 2477 | O | GLY | B | 150 | 2.488 | 28.929 | 25.708 | 1.00 | 261.29 | A | O |
| ATOM | 2478 | N | VAL | B | 151 | 1.135 | 28.911 | 27.541 | 1.00 | 269.76 | A | N |
| ATOM | 2479 | CA | VAL | B | 151 | 2.114 | 28.279 | 28.413 | 1.00 | 252.40 | A | C |
| ATOM | 2480 | CB | VAL | B | 151 | 1.690 | 28.374 | 29.887 | 1.00 | 249.85 | A | C |
| ATOM | 2481 | CG1 | VAL | B | 151 | 2.130 | 27.140 | 30.657 | 1.00 | 263.98 | A | C |
| ATOM | 2482 | CG2 | VAL | B | 151 | 2.248 | 29.642 | 30.518 | 1.00 | 217.09 | A | C |
| ATOM | 2483 | C | VAL | B | 151 | 2.367 | 26.829 | 28.006 | 1.00 | 208.66 | A | C |
| ATOM | 2484 | O | VAL | B | 151 | 3.498 | 26.443 | 27.710 | 1.00 | 225.83 | A | O |
| ATOM | 2485 | N | LEU | B | 152 | 1.311 | 26.034 | 28.003 | 1.00 | 239.63 | A | N |
| ATOM | 2486 | CA | LEU | B | 152 | 1.407 | 24.664 | 27.488 | 1.00 | 221.61 | A | C |
| ATOM | 2487 | CB | LEU | B | 152 | 0.032 | 24.045 | 27.321 | 1.00 | 229.00 | A | C |
| ATOM | 2488 | CG | LEU | B | 152 | −0.317 | 22.929 | 28.302 | 1.00 | 222.60 | A | C |
| ATOM | 2489 | CD1 | LEU | B | 152 | −1.731 | 22.449 | 28.001 | 1.00 | 223.05 | A | C |
| ATOM | 2490 | CD2 | LEU | B | 152 | 0.668 | 21.776 | 28.244 | 1.00 | 214.65 | A | C |
| ATOM | 2491 | C | LEU | B | 152 | 2.172 | 24.613 | 26.186 | 1.00 | 229.10 | A | C |
| ATOM | 2492 | O | LEU | B | 152 | 3.191 | 23.950 | 26.101 | 1.00 | 229.19 | A | O |
| ATOM | 2493 | N | TRP | B | 153 | 1.696 | 25.345 | 25.188 | 1.00 | 240.18 | A | N |
| ATOM | 2494 | CA | TRP | B | 153 | 2.288 | 25.320 | 23.845 | 1.00 | 234.67 | A | C |
| ATOM | 2495 | CB | TRP | B | 153 | 1.536 | 26.279 | 22.922 | 1.00 | 235.88 | A | C |
| ATOM | 2496 | CG | TRP | B | 153 | 0.350 | 25.627 | 22.265 | 1.00 | 190.36 | A | C |
| ATOM | 2497 | CD1 | TRP | B | 153 | 0.126 | 25.455 | 20.901 | 1.00 | 216.00 | A | C |
| ATOM | 2498 | NE1 | TRP | B | 153 | −1.039 | 24.771 | 20.683 | 1.00 | 193.61 | A | N |
| ATOM | 2499 | CE2 | TRP | B | 153 | −1.630 | 24.434 | 21.841 | 1.00 | 204.83 | A | C |
| ATOM | 2500 | CD2 | TRP | B | 153 | −0.781 | 24.949 | 22.922 | 1.00 | 199.03 | A | C |
| ATOM | 2501 | CE3 | TRP | B | 153 | −1.163 | 24.731 | 24.244 | 1.00 | 217.72 | A | C |
| ATOM | 2502 | CZ3 | TRP | B | 153 | −2.347 | 24.033 | 24.485 | 1.00 | 223.45 | A | C |
| ATOM | 2503 | CH2 | TRP | B | 153 | −3.149 | 23.558 | 23.430 | 1.00 | 241.03 | A | C |
| ATOM | 2504 | CZ2 | TRP | B | 153 | −2.803 | 23.749 | 22.092 | 1.00 | 222.30 | A | C |
| ATOM | 2505 | C | TRP | B | 153 | 3.746 | 25.623 | 23.857 | 1.00 | 197.44 | A | C |
| ATOM | 2506 | O | TRP | B | 153 | 4.533 | 24.983 | 23.163 | 1.00 | 210.99 | A | O |
| ATOM | 2507 | N | LEU | B | 154 | 4.116 | 26.594 | 24.676 | 1.00 | 187.73 | A | N |
| ATOM | 2508 | CA | LEU | B | 154 | 5.496 | 27.019 | 24.797 | 1.00 | 211.19 | A | C |
| ATOM | 2509 | CB | LEU | B | 154 | 5.568 | 28.274 | 25.676 | 1.00 | 215.20 | A | C |
| ATOM | 2510 | CG | LEU | B | 154 | 6.861 | 29.059 | 25.575 | 1.00 | 237.94 | A | C |
| ATOM | 2511 | CD1 | LEU | B | 154 | 7.333 | 29.183 | 24.142 | 1.00 | 217.50 | A | C |
| ATOM | 2512 | CD2 | LEU | B | 154 | 6.647 | 30.431 | 26.172 | 1.00 | 288.66 | A | C |
| ATOM | 2513 | C | LEU | B | 154 | 6.398 | 25.900 | 25.329 | 1.00 | 229.91 | A | C |
| ATOM | 2514 | O | LEU | B | 154 | 7.556 | 25.784 | 24.927 | 1.00 | 207.12 | A | O |
| ATOM | 2515 | N | THR | B | 155 | 5.853 | 25.064 | 26.211 | 1.00 | 229.22 | A | N |
| ATOM | 2516 | CA | THR | B | 155 | 6.602 | 23.952 | 26.789 | 1.00 | 197.14 | A | C |
| ATOM | 2517 | CB | THR | B | 155 | 5.738 | 23.091 | 27.726 | 1.00 | 192.66 | A | C |
| ATOM | 2518 | OG1 | THR | B | 155 | 4.722 | 22.437 | 26.975 | 1.00 | 215.46 | A | O |
| ATOM | 2519 | CG2 | THR | B | 155 | 5.071 | 23.949 | 28.799 | 1.00 | 194.62 | A | C |
| ATOM | 2520 | C | THR | B | 155 | 7.222 | 23.091 | 25.723 | 1.00 | 213.83 | A | C |
| ATOM | 2521 | O | THR | B | 155 | 8.408 | 22.786 | 25.778 | 1.00 | 223.36 | A | O |
| ATOM | 2522 | N | ALA | B | 156 | 6.410 | 22.717 | 24.739 | 1.00 | 185.81 | A | N |
| ATOM | 2523 | CA | ALA | B | 156 | 6.877 | 21.976 | 23.575 | 1.00 | 187.55 | A | C |
| ATOM | 2524 | CB | ALA | B | 156 | 5.754 | 21.836 | 22.577 | 1.00 | 223.28 | A | C |
| ATOM | 2525 | C | ALA | B | 156 | 8.070 | 22.682 | 22.943 | 1.00 | 187.14 | A | C |
| ATOM | 2526 | O | ALA | B | 156 | 8.962 | 22.028 | 22.425 | 1.00 | 192.68 | A | O |
| ATOM | 2527 | N | PHE | B | 157 | 8.097 | 24.012 | 23.010 | 1.00 | 207.67 | A | N |
| ATOM | 2528 | CA | PHE | B | 157 | 9.169 | 24.784 | 22.395 | 1.00 | 209.11 | A | C |
| ATOM | 2529 | CB | PHE | B | 157 | 8.832 | 26.270 | 22.323 | 1.00 | 229.95 | A | C |
| ATOM | 2530 | CG | PHE | B | 157 | 10.026 | 27.130 | 22.054 | 1.00 | 228.34 | A | C |
| ATOM | 2531 | CD1 | PHE | B | 157 | 10.581 | 27.184 | 20.791 | 1.00 | 238.24 | A | C |
| ATOM | 2532 | CE1 | PHE | B | 157 | 11.681 | 27.974 | 20.535 | 1.00 | 258.18 | A | C |
| ATOM | 2533 | CZ | PHE | B | 157 | 12.265 | 28.692 | 21.555 | 1.00 | 262.53 | A | C |
| ATOM | 2534 | CE2 | PHE | B | 157 | 11.728 | 28.631 | 22.828 | 1.00 | 276.76 | A | C |
| ATOM | 2535 | CD2 | PHE | B | 157 | 10.613 | 27.855 | 23.069 | 1.00 | 217.75 | A | C |
| ATOM | 2536 | C | PHE | B | 157 | 10.481 | 24.623 | 23.119 | 1.00 | 219.73 | A | C |
| ATOM | 2537 | O | PHE | B | 157 | 11.422 | 24.106 | 22.558 | 1.00 | 229.12 | A | O |
| ATOM | 2538 | N | ILE | B | 158 | 10.549 | 25.095 | 24.356 | 1.00 | 240.83 | A | N |
| ATOM | 2539 | CA | ILE | B | 158 | 11.791 | 25.032 | 25.125 | 1.00 | 252.19 | A | C |
| ATOM | 2540 | CB | ILE | B | 158 | 11.609 | 25.488 | 26.588 | 1.00 | 277.38 | A | C |
| ATOM | 2541 | CG1 | ILE | B | 158 | 10.363 | 24.853 | 27.210 | 1.00 | 270.52 | A | C |
| ATOM | 2542 | CD1 | ILE | B | 158 | 10.426 | 24.638 | 28.705 | 1.00 | 293.83 | A | C |
| ATOM | 2543 | CG2 | ILE | B | 158 | 11.532 | 27.000 | 26.656 | 1.00 | 278.37 | A | C |
| ATOM | 2544 | C | ILE | B | 158 | 12.349 | 23.625 | 25.103 | 1.00 | 240.06 | A | C |
| ATOM | 2545 | O | ILE | B | 158 | 13.501 | 23.415 | 24.730 | 1.00 | 224.79 | A | O |
| ATOM | 2546 | N | GLU | B | 159 | 11.501 | 22.663 | 25.462 | 1.00 | 228.77 | A | N |
| ATOM | 2547 | CA | GLU | B | 159 | 11.916 | 21.283 | 25.639 | 1.00 | 222.83 | A | C |
| ATOM | 2548 | CB | GLU | B | 159 | 10.762 | 20.421 | 26.128 | 1.00 | 230.09 | A | C |
| ATOM | 2549 | CG | GLU | B | 159 | 11.201 | 19.019 | 26.514 | 1.00 | 226.38 | A | C |
| ATOM | 2550 | CD | GLU | B | 159 | 10.303 | 18.391 | 27.567 | 1.00 | 300.13 | A | C |
| ATOM | 2551 | OE1 | GLU | B | 159 | 9.064 | 18.552 | 27.515 | 1.00 | 411.50 | A | O |
| ATOM | 2552 | OE2 | GLU | B | 159 | 10.850 | 17.743 | 28.478 | 1.00 | 322.97 | A | O |
| ATOM | 2553 | C | GLU | B | 159 | 12.503 | 20.675 | 24.399 | 1.00 | 216.63 | A | C |
| ATOM | 2554 | O | GLU | B | 159 | 13.507 | 19.974 | 24.455 | 1.00 | 227.14 | A | O |
| ATOM | 2555 | N | ASN | B | 160 | 11.881 | 20.978 | 23.277 | 1.00 | 201.09 | A | N |
| ATOM | 2556 | CA | ASN | B | 160 | 12.202 | 20.287 | 22.058 | 1.00 | 200.88 | A | C |
| ATOM | 2557 | CB | ASN | B | 160 | 10.925 | 19.980 | 21.278 | 1.00 | 210.10 | A | C |
| ATOM | 2558 | CG | ASN | B | 160 | 10.233 | 18.732 | 21.767 | 1.00 | 179.01 | A | C |
| ATOM | 2559 | OD1 | ASN | B | 160 | 10.879 | 17.716 | 22.015 | 1.00 | 178.92 | A | O |
| ATOM | 2560 | ND2 | ASN | B | 160 | 8.910 | 18.794 | 21.894 | 1.00 | 181.03 | A | N |
| ATOM | 2561 | C | ASN | B | 160 | 13.222 | 20.954 | 21.153 | 1.00 | 237.20 | A | C |
| ATOM | 2562 | O | ASN | B | 160 | 14.062 | 20.265 | 20.573 | 1.00 | 228.99 | A | O |
| ATOM | 2563 | N | ILE | B | 161 | 13.161 | 22.278 | 21.027 | 1.00 | 243.70 | A | N |
| ATOM | 2564 | CA | ILE | B | 161 | 13.860 | 22.926 | 19.916 | 1.00 | 268.11 | A | C |
| ATOM | 2565 | CB | ILE | B | 161 | 13.078 | 24.088 | 19.274 | 1.00 | 260.45 | A | C |
| ATOM | 2566 | CG1 | ILE | B | 161 | 13.739 | 24.452 | 17.942 | 1.00 | 261.58 | A | C |
| ATOM | 2567 | CD1 | ILE | B | 161 | 13.932 | 23.279 | 16.982 | 1.00 | 257.23 | A | C |
| ATOM | 2568 | CG2 | ILE | B | 161 | 13.034 | 25.295 | 20.195 | 1.00 | 251.07 | A | C |
| ATOM | 2569 | C | ILE | B | 161 | 15.324 | 23.298 | 20.131 | 1.00 | 269.27 | A | C |
| ATOM | 2570 | O | ILE | B | 161 | 16.161 | 23.028 | 19.269 | 1.00 | 285.25 | A | O |
| ATOM | 2571 | N | LEU | B | 162 | 15.623 | 23.961 | 21.238 | 1.00 | 262.87 | A | N |
| ATOM | 2572 | CA | LEU | B | 162 | 17.005 | 24.212 | 21.586 | 1.00 | 286.97 | A | C |
| ATOM | 2573 | CB | LEU | B | 162 | 17.675 | 25.213 | 20.625 | 1.00 | 278.61 | A | C |
| ATOM | 2574 | CG | LEU | B | 162 | 19.128 | 24.876 | 20.256 | 1.00 | 298.36 | A | C |
| ATOM | 2575 | CD1 | LEU | B | 162 | 19.262 | 23.442 | 19.764 | 1.00 | 292.40 | A | C |
| ATOM | 2576 | CD2 | LEU | B | 162 | 19.711 | 25.842 | 19.231 | 1.00 | 287.05 | A | C |
| ATOM | 2577 | C | LEU | B | 162 | 17.064 | 24.683 | 23.019 | 1.00 | 284.15 | A | C |
| ATOM | 2578 | O | LEU | B | 162 | 17.717 | 25.681 | 23.335 | 1.00 | 335.27 | A | O |
| ATOM | 2579 | N | LYS | B | 163 | 16.347 | 23.974 | 23.888 | 1.00 | 292.44 | A | N |
| ATOM | 2580 | CA | LYS | B | 163 | 16.558 | 24.126 | 25.325 | 1.00 | 314.61 | A | C |
| ATOM | 2581 | CB | LYS | B | 163 | 15.515 | 25.014 | 25.993 | 1.00 | 304.45 | A | C |
| ATOM | 2582 | CG | LYS | B | 163 | 15.676 | 24.943 | 27.499 | 1.00 | 297.64 | A | C |
| ATOM | 2583 | CD | LYS | B | 163 | 14.648 | 25.736 | 28.248 | 1.00 | 329.63 | A | C |
| ATOM | 2584 | CE | LYS | B | 163 | 15.121 | 26.000 | 29.678 | 1.00 | 384.38 | A | C |
| ATOM | 2585 | NZ | LYS | B | 163 | 15.066 | 27.431 | 30.120 | 1.00 | 440.58 | A | N |
| ATOM | 2586 | C | LYS | B | 163 | 16.671 | 22.836 | 26.119 | 1.00 | 286.96 | A | C |
| ATOM | 2587 | O | LYS | B | 163 | 17.356 | 22.817 | 27.143 | 1.00 | 334.88 | A | O |
| ATOM | 2588 | N | ILE | B | 164 | 15.975 | 21.785 | 25.692 | 1.00 | 249.65 | A | N |
| ATOM | 2589 | CA | ILE | B | 164 | 16.014 | 20.463 | 26.359 | 1.00 | 254.52 | A | C |
| ATOM | 2590 | CB | ILE | B | 164 | 17.184 | 19.576 | 25.842 | 1.00 | 271.93 | A | C |
| ATOM | 2591 | CG1 | ILE | B | 164 | 18.545 | 20.277 | 26.000 | 1.00 | 295.90 | A | C |
| ATOM | 2592 | CD1 | ILE | B | 164 | 19.754 | 19.360 | 25.890 | 1.00 | 273.94 | A | C |
| ATOM | 2593 | CG2 | ILE | B | 164 | 16.958 | 19.182 | 24.389 | 1.00 | 265.90 | A | C |
| ATOM | 2594 | C | ILE | B | 164 | 15.923 | 20.462 | 27.913 | 1.00 | 256.04 | A | C |
| ATOM | 2595 | O | ILE | B | 164 | 16.857 | 20.046 | 28.612 | 1.00 | 282.61 | A | O |
| ATOM | 2596 | N | PRO | B | 165 | 14.796 | 20.959 | 28.450 | 1.00 | 240.71 | A | N |
| ATOM | 2597 | CA | PRO | B | 165 | 14.494 | 20.922 | 29.870 | 1.00 | 223.82 | A | C |
| ATOM | 2598 | CB | PRO | B | 165 | 13.749 | 22.231 | 30.078 | 1.00 | 268.14 | A | C |
| ATOM | 2599 | CG | PRO | B | 165 | 12.965 | 22.350 | 28.833 | 1.00 | 278.49 | A | C |
| ATOM | 2600 | CD | PRO | B | 165 | 13.939 | 21.941 | 27.770 | 1.00 | 244.28 | A | C |
| ATOM | 2601 | C | PRO | B | 165 | 13.578 | 19.766 | 30.260 | 1.00 | 236.02 | A | C |
| ATOM | 2602 | O | PRO | B | 165 | 13.327 | 18.897 | 29.462 | 1.00 | 247.71 | A | O |
| ATOM | 2603 | N | LEU | B | 166 | 13.048 | 19.822 | 31.476 | 1.00 | 302.90 | A | N |
| ATOM | 2604 | CA | LEU | B | 166 | 12.374 | 18.711 | 32.135 | 1.00 | 327.18 | A | C |
| ATOM | 2605 | CB | LEU | B | 166 | 12.727 | 18.721 | 33.637 | 1.00 | 330.50 | A | C |
| ATOM | 2606 | CG | LEU | B | 166 | 13.935 | 19.496 | 34.233 | 1.00 | 306.53 | A | C |
| ATOM | 2607 | CD1 | LEU | B | 166 | 13.600 | 20.150 | 35.581 | 1.00 | 333.00 | A | C |
| ATOM | 2608 | CD2 | LEU | B | 166 | 15.183 | 18.623 | 34.329 | 1.00 | 353.19 | A | C |
| ATOM | 2609 | C | LEU | B | 166 | 10.851 | 18.781 | 31.963 | 1.00 | 400.62 | A | C |
| ATOM | 2610 | O | LEU | B | 166 | 10.278 | 19.847 | 31.681 | 1.00 | 428.70 | A | O |
| ATOM | 2611 | N | GLY | B | 167 | 10.192 | 17.658 | 32.192 | 1.00 | 348.95 | A | N |
| ATOM | 2612 | CA | GLY | B | 167 | 8.831 | 17.583 | 31.764 | 1.00 | 300.57 | A | C |
| ATOM | 2613 | C | GLY | B | 167 | 7.718 | 16.902 | 32.512 | 1.00 | 287.46 | A | C |
| ATOM | 2614 | O | GLY | B | 167 | 6.573 | 16.993 | 32.085 | 1.00 | 296.53 | A | O |
| ATOM | 2615 | N | LYS | B | 168 | 8.016 | 16.208 | 33.599 | 1.00 | 257.82 | A | N |
| ATOM | 2616 | CA | LYS | B | 168 | 6.992 | 15.390 | 34.293 | 1.00 | 280.26 | A | C |
| ATOM | 2617 | CB | LYS | B | 168 | 7.584 | 14.694 | 35.539 | 1.00 | 247.42 | A | C |
| ATOM | 2618 | CG | LYS | B | 168 | 6.629 | 14.303 | 36.681 | 1.00 | 257.42 | A | C |
| ATOM | 2619 | CD | LYS | B | 168 | 7.420 | 13.968 | 37.946 | 1.00 | 271.41 | A | C |
| ATOM | 2620 | CE | LYS | B | 168 | 6.813 | 14.473 | 39.259 | 1.00 | 359.02 | A | C |
| ATOM | 2621 | NZ | LYS | B | 168 | 7.879 | 14.494 | 40.300 | 1.00 | 363.25 | A | N |
| ATOM | 2622 | C | LYS | B | 168 | 5.624 | 16.056 | 34.590 | 1.00 | 272.10 | A | C |
| ATOM | 2623 | O | LYS | B | 168 | 4.629 | 15.338 | 34.746 | 1.00 | 277.57 | A | O |
| ATOM | 2624 | N | PHE | B | 169 | 5.578 | 17.399 | 34.668 | 1.00 | 276.90 | A | N |
| ATOM | 2625 | CA | PHE | B | 169 | 4.300 | 18.118 | 34.898 | 1.00 | 305.49 | A | C |
| ATOM | 2626 | CB | PHE | B | 169 | 4.409 | 19.181 | 36.057 | 1.00 | 340.22 | A | C |
| ATOM | 2627 | CG | PHE | B | 169 | 3.810 | 18.715 | 37.392 | 1.00 | 370.75 | A | C |
| ATOM | 2628 | CD1 | PHE | B | 169 | 4.438 | 17.724 | 38.157 | 1.00 | 387.83 | A | C |
| ATOM | 2629 | CE1 | PHE | B | 169 | 3.903 | 17.275 | 39.359 | 1.00 | 362.41 | A | C |
| ATOM | 2630 | CZ | PHE | B | 169 | 2.683 | 17.770 | 39.766 | 1.00 | 297.03 | A | C |
| ATOM | 2631 | CE2 | PHE | B | 169 | 2.035 | 18.760 | 39.031 | 1.00 | 328.49 | A | C |
| ATOM | 2632 | CD2 | PHE | B | 169 | 2.595 | 19.224 | 37.846 | 1.00 | 369.08 | A | C |
| ATOM | 2633 | C | PHE | B | 169 | 3.684 | 18.671 | 33.584 | 1.00 | 267.69 | A | C |
| ATOM | 2634 | O | PHE | B | 169 | 2.504 | 19.010 | 33.541 | 1.00 | 259.96 | A | O |
| ATOM | 2635 | N | THR | B | 170 | 4.479 | 18.736 | 32.515 | 1.00 | 275.99 | A | N |
| ATOM | 2636 | CA | THR | B | 170 | 3.999 | 19.212 | 31.197 | 1.00 | 274.65 | A | C |
| ATOM | 2637 | CB | THR | B | 170 | 5.059 | 19.062 | 30.082 | 1.00 | 251.23 | A | C |
| ATOM | 2638 | OG1 | THR | B | 170 | 6.305 | 19.607 | 30.528 | 1.00 | 290.99 | A | O |
| ATOM | 2639 | CG2 | THR | B | 170 | 4.617 | 19.774 | 28.829 | 1.00 | 230.16 | A | C |
| ATOM | 2640 | C | THR | B | 170 | 2.722 | 18.507 | 30.755 | 1.00 | 239.68 | A | C |
| ATOM | 2641 | O | THR | B | 170 | 1.829 | 19.142 | 30.203 | 1.00 | 241.36 | A | O |
| ATOM | 2642 | N | PRO | B | 171 | 2.639 | 17.191 | 30.997 | 1.00 | 220.31 | A | N |
| ATOM | 2643 | CA | PRO | B | 171 | 1.389 | 16.455 | 30.822 | 1.00 | 226.23 | A | C |
| ATOM | 2644 | CB | PRO | B | 171 | 1.796 | 15.008 | 31.114 | 1.00 | 232.99 | A | C |
| ATOM | 2645 | CG | PRO | B | 171 | 3.142 | 15.084 | 31.751 | 1.00 | 252.30 | A | C |
| ATOM | 2646 | CD | PRO | B | 171 | 3.784 | 16.286 | 31.177 | 1.00 | 223.58 | A | C |
| ATOM | 2647 | C | PRO | B | 171 | 0.319 | 16.926 | 31.818 | 1.00 | 218.26 | A | C |
| ATOM | 2648 | O | PRO | B | 171 | −0.830 | 17.150 | 31.446 | 1.00 | 216.72 | A | O |
| ATOM | 2649 | N | TRP | B | 172 | 0.715 | 17.116 | 33.064 | 1.00 | 244.21 | A | N |
| ATOM | 2650 | CA | TRP | B | 172 | −0.226 | 17.522 | 34.116 | 1.00 | 230.00 | A | C |
| ATOM | 2651 | CB | TRP | B | 172 | 0.422 | 17.467 | 35.504 | 1.00 | 285.13 | A | C |
| ATOM | 2652 | CG | TRP | B | 172 | 0.671 | 16.022 | 35.879 | 1.00 | 302.82 | A | C |
| ATOM | 2653 | CD1 | TRP | B | 172 | 1.892 | 15.345 | 35.922 | 1.00 | 283.37 | A | C |
| ATOM | 2654 | NE1 | TRP | B | 172 | 1.698 | 14.026 | 36.249 | 1.00 | 286.30 | A | N |
| ATOM | 2655 | CE2 | TRP | B | 172 | 0.379 | 13.752 | 36.429 | 1.00 | 267.36 | A | C |
| ATOM | 2656 | CD2 | TRP | B | 172 | −0.350 | 14.999 | 36.209 | 1.00 | 266.71 | A | C |
| ATOM | 2657 | CE3 | TRP | B | 172 | −1.746 | 15.002 | 36.338 | 1.00 | 263.82 | A | C |
| ATOM | 2658 | CZ3 | TRP | B | 172 | −2.390 | 13.812 | 36.705 | 1.00 | 319.44 | A | C |
| ATOM | 2659 | CH2 | TRP | B | 172 | −1.669 | 12.623 | 36.909 | 1.00 | 273.18 | A | C |
| ATOM | 2660 | CZ2 | TRP | B | 172 | −0.271 | 12.575 | 36.794 | 1.00 | 272.78 | A | C |
| ATOM | 2661 | C | TRP | B | 172 | −0.837 | 18.835 | 33.777 | 1.00 | 250.88 | A | C |
| ATOM | 2662 | O | TRP | B | 172 | −2.058 | 19.011 | 33.866 | 1.00 | 257.51 | A | O |
| ATOM | 2663 | N | LEU | B | 173 | 0.009 | 19.766 | 33.350 | 1.00 | 249.15 | A | N |
| ATOM | 2664 | CA | LEU | B | 173 | −0.444 | 21.064 | 32.892 | 1.00 | 278.17 | A | C |
| ATOM | 2665 | CB | LEU | B | 173 | 0.724 | 21.811 | 32.254 | 1.00 | 247.86 | A | C |
| ATOM | 2666 | CG | LEU | B | 173 | 0.473 | 23.210 | 31.689 | 1.00 | 176.36 | A | C |
| ATOM | 2667 | CD1 | LEU | B | 173 | −0.206 | 24.093 | 32.672 | 1.00 | 171.18 | A | C |
| ATOM | 2668 | CD2 | LEU | B | 173 | 1.755 | 23.872 | 31.195 | 1.00 | 255.84 | A | C |
| ATOM | 2669 | C | LEU | B | 173 | −1.591 | 20.879 | 31.905 | 1.00 | 212.71 | A | C |
| ATOM | 2670 | O | LEU | B | 173 | −2.623 | 21.539 | 31.996 | 1.00 | 229.91 | A | O |
| ATOM | 2671 | N | ALA | B | 174 | −1.414 | 19.926 | 30.961 | 1.00 | 196.18 | A | N |
| ATOM | 2672 | CA | ALA | B | 174 | −2.390 | 19.675 | 29.962 | 1.00 | 204.93 | A | C |
| ATOM | 2673 | CB | ALA | B | 174 | −1.754 | 18.867 | 28.903 | 1.00 | 171.37 | A | C |
| ATOM | 2674 | C | ALA | B | 174 | −3.614 | 18.980 | 30.525 | 1.00 | 232.89 | A | C |
| ATOM | 2675 | O | ALA | B | 174 | −4.714 | 19.146 | 30.010 | 1.00 | 236.16 | A | O |
| ATOM | 2676 | N | ILE | B | 175 | −3.429 | 18.209 | 31.589 | 1.00 | 222.65 | A | N |
| ATOM | 2677 | CA | ILE | B | 175 | −4.541 | 17.469 | 32.179 | 1.00 | 230.40 | A | C |
| ATOM | 2678 | CB | ILE | B | 175 | −4.066 | 16.281 | 33.028 | 1.00 | 214.75 | A | C |
| ATOM | 2679 | CG1 | ILE | B | 175 | −3.179 | 15.359 | 32.182 | 1.00 | 238.46 | A | C |
| ATOM | 2680 | CD1 | ILE | B | 175 | −2.102 | 14.652 | 32.969 | 1.00 | 258.33 | A | C |
| ATOM | 2681 | CG2 | ILE | B | 175 | −5.276 | 15.523 | 33.580 | 1.00 | 225.44 | A | C |
| ATOM | 2682 | C | ILE | B | 175 | −5.473 | 18.384 | 32.957 | 1.00 | 221.20 | A | C |
| ATOM | 2683 | O | ILE | B | 175 | −6.663 | 18.461 | 32.660 | 1.00 | 229.48 | A | O |
| ATOM | 2684 | N | ILE | B | 176 | −4.923 | 19.076 | 33.952 | 1.00 | 232.69 | A | N |
| ATOM | 2685 | CA | ILE | B | 176 | −5.662 | 20.116 | 34.680 | 1.00 | 226.15 | A | C |
| ATOM | 2686 | CB | ILE | B | 176 | −4.766 | 20.886 | 35.658 | 1.00 | 222.94 | A | C |
| ATOM | 2687 | CG1 | ILE | B | 176 | −3.593 | 21.535 | 34.915 | 1.00 | 215.59 | A | C |
| ATOM | 2688 | CD1 | ILE | B | 176 | −2.577 | 22.216 | 35.808 | 1.00 | 223.71 | A | C |
| ATOM | 2689 | CG2 | ILE | B | 176 | −4.285 | 19.966 | 36.778 | 1.00 | 217.03 | A | C |
| ATOM | 2690 | C | ILE | B | 176 | −6.301 | 21.095 | 33.699 | 1.00 | 230.71 | A | C |
| ATOM | 2691 | O | ILE | B | 176 | −7.501 | 21.381 | 33.798 | 1.00 | 239.07 | A | O |
| ATOM | 2692 | N | GLU | B | 177 | −5.500 | 21.572 | 32.744 | 1.00 | 219.87 | A | N |
| ATOM | 2693 | CA | GLU | B | 177 | −5.962 | 22.516 | 31.740 | 1.00 | 206.23 | A | C |
| ATOM | 2694 | CB | GLU | B | 177 | −4.825 | 22.954 | 30.821 | 1.00 | 220.44 | A | C |
| ATOM | 2695 | CG | GLU | B | 177 | −4.290 | 24.338 | 31.143 | 1.00 | 244.75 | A | C |
| ATOM | 2696 | CD | GLU | B | 177 | −3.378 | 24.857 | 30.054 | 1.00 | 257.49 | A | C |
| ATOM | 2697 | OE1 | GLU | B | 177 | −2.222 | 25.207 | 30.369 | 1.00 | 229.44 | A | O |
| ATOM | 2698 | OE2 | GLU | B | 177 | −3.800 | 24.882 | 28.873 | 1.00 | 253.40 | A | O |
| ATOM | 2699 | C | GLU | B | 177 | −7.111 | 21.950 | 30.930 | 1.00 | 226.33 | A | C |
| ATOM | 2700 | O | GLU | B | 177 | −7.927 | 22.698 | 30.396 | 1.00 | 213.39 | A | O |
| ATOM | 2701 | N | GLY | B | 178 | −7.160 | 20.624 | 30.848 | 1.00 | 217.21 | A | N |
| ATOM | 2702 | CA | GLY | B | 178 | −8.280 | 19.923 | 30.241 | 1.00 | 222.80 | A | C |
| ATOM | 2703 | C | GLY | B | 178 | −9.546 | 20.076 | 31.054 | 1.00 | 226.73 | A | C |
| ATOM | 2704 | O | GLY | B | 178 | −10.525 | 20.654 | 30.577 | 1.00 | 216.94 | A | O |
| ATOM | 2705 | N | ILE | B | 179 | −9.520 | 19.587 | 32.296 | 1.00 | 261.77 | A | N |
| ATOM | 2706 | CA | ILE | B | 179 | −10.673 | 19.724 | 33.199 | 1.00 | 280.87 | A | C |
| ATOM | 2707 | CB | ILE | B | 179 | −10.431 | 19.128 | 34.599 | 1.00 | 258.86 | A | C |
| ATOM | 2708 | CG1 | ILE | B | 179 | −10.122 | 17.639 | 34.551 | 1.00 | 288.22 | A | C |
| ATOM | 2709 | CD1 | ILE | B | 179 | −9.725 | 17.095 | 35.929 | 1.00 | 310.41 | A | C |
| ATOM | 2710 | CG2 | ILE | B | 179 | −11.679 | 19.312 | 35.454 | 1.00 | 269.44 | A | C |
| ATOM | 2711 | C | ILE | B | 179 | −11.072 | 21.184 | 33.410 | 1.00 | 241.55 | A | C |
| ATOM | 2712 | O | ILE | B | 179 | −12.259 | 21.543 | 33.358 | 1.00 | 239.92 | A | O |
| ATOM | 2713 | N | LEU | B | 180 | −10.074 | 22.019 | 33.652 | 1.00 | 217.74 | A | N |
| ATOM | 2714 | CA | LEU | B | 180 | −10.316 | 23.405 | 33.981 | 1.00 | 252.43 | A | C |
| ATOM | 2715 | CB | LEU | B | 180 | −9.131 | 23.988 | 34.755 | 1.00 | 244.20 | A | C |
| ATOM | 2716 | CG | LEU | B | 180 | −9.168 | 24.258 | 36.263 | 1.00 | 231.99 | A | C |
| ATOM | 2717 | CD1 | LEU | B | 180 | −9.247 | 22.972 | 37.081 | 1.00 | 234.70 | A | C |
| ATOM | 2718 | CD2 | LEU | B | 180 | −7.906 | 25.051 | 36.567 | 1.00 | 254.38 | A | C |
| ATOM | 2719 | C | LEU | B | 180 | −10.583 | 24.271 | 32.768 | 1.00 | 239.72 | A | C |
| ATOM | 2720 | O | LEU | B | 180 | −11.686 | 24.791 | 32.603 | 1.00 | 237.93 | A | O |
| ATOM | 2721 | N | THR | B | 181 | −9.559 | 24.442 | 31.941 | 1.00 | 224.37 | A | N |
| ATOM | 2722 | CA | THR | B | 181 | −9.500 | 25.567 | 31.006 | 1.00 | 211.06 | A | C |
| ATOM | 2723 | CB | THR | B | 181 | −8.059 | 25.834 | 30.529 | 1.00 | 240.06 | A | C |
| ATOM | 2724 | OG1 | THR | B | 181 | −7.148 | 25.678 | 31.619 | 1.00 | 271.00 | A | O |
| ATOM | 2725 | CG2 | THR | B | 181 | −7.936 | 27.243 | 30.022 | 1.00 | 194.51 | A | C |
| ATOM | 2726 | C | THR | B | 181 | −10.407 | 25.401 | 29.799 | 1.00 | 197.68 | A | C |
| ATOM | 2727 | O | THR | B | 181 | −11.031 | 26.348 | 29.341 | 1.00 | 228.84 | A | O |
| ATOM | 2728 | N | ALA | B | 182 | −10.499 | 24.192 | 29.309 | 1.00 | 210.91 | A | N |
| ATOM | 2729 | CA | ALA | B | 182 | −11.343 | 23.931 | 28.163 | 1.00 | 168.88 | A | C |
| ATOM | 2730 | CB | ALA | B | 182 | −10.618 | 22.986 | 27.196 | 1.00 | 169.30 | A | C |
| ATOM | 2731 | C | ALA | B | 182 | −12.679 | 23.339 | 28.551 | 1.00 | 214.78 | A | C |
| ATOM | 2732 | O | ALA | B | 182 | −13.709 | 23.724 | 28.004 | 1.00 | 217.42 | A | O |
| ATOM | 2733 | N | TRP | B | 183 | −12.656 | 22.413 | 29.507 | 1.00 | 218.92 | A | N |
| ATOM | 2734 | CA | TRP | B | 183 | −13.775 | 21.515 | 29.737 | 1.00 | 205.48 | A | C |
| ATOM | 2735 | CB | TRP | B | 183 | −13.361 | 20.385 | 30.660 | 1.00 | 220.70 | A | C |
| ATOM | 2736 | CG | TRP | B | 183 | −14.410 | 19.319 | 30.859 | 1.00 | 241.91 | A | C |
| ATOM | 2737 | CD1 | TRP | B | 183 | −15.767 | 19.378 | 30.545 | 1.00 | 279.98 | A | C |
| ATOM | 2738 | NE1 | TRP | B | 183 | −16.401 | 18.211 | 30.890 | 1.00 | 247.16 | A | N |
| ATOM | 2739 | CE2 | TRP | B | 183 | −15.527 | 17.334 | 31.441 | 1.00 | 230.36 | A | C |
| ATOM | 2740 | CD2 | TRP | B | 183 | −14.213 | 17.983 | 31.446 | 1.00 | 240.06 | A | C |
| ATOM | 2741 | CE3 | TRP | B | 183 | −13.129 | 17.307 | 31.997 | 1.00 | 262.15 | A | C |
| ATOM | 2742 | CZ3 | TRP | B | 183 | −13.334 | 16.017 | 32.502 | 1.00 | 272.99 | A | C |
| ATOM | 2743 | CH2 | TRP | B | 183 | −14.591 | 15.399 | 32.454 | 1.00 | 207.87 | A | C |
| ATOM | 2744 | CZ2 | TRP | B | 183 | −15.712 | 16.052 | 31.939 | 1.00 | 223.01 | A | C |
| ATOM | 2745 | C | TRP | B | 183 | −14.973 | 22.211 | 30.309 | 1.00 | 203.98 | A | C |
| ATOM | 2746 | O | TRP | B | 183 | −16.032 | 22.224 | 29.678 | 1.00 | 257.78 | A | O |
| ATOM | 2747 | N | ILE | B | 184 | −14.840 | 22.783 | 31.523 | 1.00 | 197.12 | A | N |
| ATOM | 2748 | CA | ILE | B | 184 | −15.925 | 23.410 | 32.210 | 1.00 | 200.22 | A | C |
| ATOM | 2749 | CB | ILE | B | 184 | −15.514 | 23.734 | 33.666 | 1.00 | 210.58 | A | C |
| ATOM | 2750 | CG1 | ILE | B | 184 | −15.424 | 22.417 | 34.452 | 1.00 | 227.32 | A | C |
| ATOM | 2751 | CD1 | ILE | B | 184 | −14.443 | 22.426 | 35.600 | 1.00 | 227.70 | A | C |
| ATOM | 2752 | CG2 | ILE | B | 184 | −16.475 | 24.726 | 34.363 | 1.00 | 157.65 | A | C |
| ATOM | 2753 | C | ILE | B | 184 | −16.429 | 24.636 | 31.441 | 1.00 | 233.32 | A | C |
| ATOM | 2754 | O | ILE | B | 184 | −17.632 | 24.743 | 31.230 | 1.00 | 272.60 | A | O |
| ATOM | 2755 | N | PRO | B | 185 | −15.518 | 25.536 | 30.989 | 1.00 | 261.24 | A | N |
| ATOM | 2756 | CA | PRO | B | 185 | −15.907 | 26.745 | 30.232 | 1.00 | 234.42 | A | C |
| ATOM | 2757 | CB | PRO | B | 185 | −14.565 | 27.286 | 29.727 | 1.00 | 232.09 | A | C |
| ATOM | 2758 | CG | PRO | B | 185 | −13.629 | 26.899 | 30.813 | 1.00 | 224.31 | A | C |
| ATOM | 2759 | CD | PRO | B | 185 | −14.056 | 25.496 | 31.184 | 1.00 | 247.92 | A | C |
| ATOM | 2760 | C | PRO | B | 185 | −16.794 | 26.420 | 29.039 | 1.00 | 280.66 | A | C |
| ATOM | 2761 | O | PRO | B | 185 | −17.615 | 27.236 | 28.648 | 1.00 | 248.83 | A | O |
| ATOM | 2762 | N | ALA | B | 186 | −16.638 | 25.230 | 28.491 | 1.00 | 242.09 | A | N |
| ATOM | 2763 | CA | ALA | B | 186 | −17.484 | 24.774 | 27.405 | 1.00 | 228.14 | A | C |
| ATOM | 2764 | CB | ALA | B | 186 | −16.809 | 23.651 | 26.638 | 1.00 | 231.03 | A | C |
| ATOM | 2765 | C | ALA | B | 186 | −18.819 | 24.317 | 27.966 | 1.00 | 235.31 | A | C |
| ATOM | 2766 | O | ALA | B | 186 | −19.868 | 24.786 | 27.539 | 1.00 | 218.96 | A | O |
| ATOM | 2767 | N | TRP | B | 187 | −18.770 | 23.413 | 28.940 | 1.00 | 220.92 | A | N |
| ATOM | 2768 | CA | TRP | B | 187 | −19.968 | 22.923 | 29.585 | 1.00 | 207.53 | A | C |
| ATOM | 2769 | CB | TRP | B | 187 | −19.644 | 21.779 | 30.529 | 1.00 | 211.55 | A | C |
| ATOM | 2770 | CG | TRP | B | 187 | −20.881 | 21.211 | 31.162 | 1.00 | 223.32 | A | C |
| ATOM | 2771 | CD1 | TRP | B | 187 | −21.415 | 21.538 | 32.403 | 1.00 | 266.96 | A | C |
| ATOM | 2772 | NE1 | TRP | B | 187 | −22.570 | 20.837 | 32.640 | 1.00 | 292.00 | A | N |
| ATOM | 2773 | CE2 | TRP | B | 187 | −22.864 | 20.032 | 31.594 | 1.00 | 274.16 | A | C |
| ATOM | 2774 | CD2 | TRP | B | 187 | −21.809 | 20.222 | 30.596 | 1.00 | 216.71 | A | C |
| ATOM | 2775 | CE3 | TRP | B | 187 | −21.877 | 19.518 | 29.416 | 1.00 | 235.74 | A | C |
| ATOM | 2776 | CZ3 | TRP | B | 187 | −22.942 | 18.629 | 29.222 | 1.00 | 276.75 | A | C |
| ATOM | 2777 | CH2 | TRP | B | 187 | −23.936 | 18.450 | 30.189 | 1.00 | 277.39 | A | C |
| ATOM | 2778 | CZ2 | TRP | B | 187 | −23.921 | 19.153 | 31.390 | 1.00 | 266.68 | A | C |
| ATOM | 2779 | C | TRP | B | 187 | −20.673 | 24.067 | 30.290 | 1.00 | 208.84 | A | C |
| ATOM | 2780 | O | TRP | B | 187 | −21.873 | 24.024 | 30.539 | 1.00 | 217.18 | A | O |
| ATOM | 2781 | N | LEU | B | 188 | −19.910 | 25.113 | 30.578 | 1.00 | 233.44 | A | N |
| ATOM | 2782 | CA | LEU | B | 188 | −20.436 | 26.331 | 31.183 | 1.00 | 233.90 | A | C |
| ATOM | 2783 | CB | LEU | B | 188 | −19.441 | 26.849 | 32.221 | 1.00 | 223.08 | A | C |
| ATOM | 2784 | CG | LEU | B | 188 | −19.587 | 28.224 | 32.865 | 1.00 | 257.41 | A | C |
| ATOM | 2785 | CD1 | LEU | B | 188 | −19.021 | 28.163 | 34.267 | 1.00 | 265.23 | A | C |
| ATOM | 2786 | CD2 | LEU | B | 188 | −18.867 | 29.283 | 32.048 | 1.00 | 262.67 | A | C |
| ATOM | 2787 | C | LEU | B | 188 | −20.703 | 27.384 | 30.110 | 1.00 | 237.27 | A | C |
| ATOM | 2788 | O | LEU | B | 188 | −21.259 | 28.427 | 30.388 | 1.00 | 321.46 | A | O |
| ATOM | 2789 | N | LEU | B | 189 | −20.274 | 27.108 | 28.878 | 1.00 | 237.41 | A | N |
| ATOM | 2790 | CA | LEU | B | 189 | −20.604 | 27.973 | 27.746 | 1.00 | 237.59 | A | C |
| ATOM | 2791 | CB | LEU | B | 189 | −19.595 | 27.809 | 26.617 | 1.00 | 246.18 | A | C |
| ATOM | 2792 | CG | LEU | B | 189 | −19.198 | 29.084 | 25.870 | 1.00 | 224.92 | A | C |
| ATOM | 2793 | CD1 | LEU | B | 189 | −18.647 | 30.134 | 26.807 | 1.00 | 219.79 | A | C |
| ATOM | 2794 | CD2 | LEU | B | 189 | −18.144 | 28.746 | 24.826 | 1.00 | 240.48 | A | C |
| ATOM | 2795 | C | LEU | B | 189 | −21.998 | 27.642 | 27.254 | 1.00 | 221.43 | A | C |
| ATOM | 2796 | O | LEU | B | 189 | −22.644 | 28.454 | 26.604 | 1.00 | 221.82 | A | O |
| ATOM | 2797 | N | PHE | B | 190 | −22.456 | 26.437 | 27.577 | 1.00 | 223.49 | A | N |
| ATOM | 2798 | CA | PHE | B | 190 | −23.819 | 26.027 | 27.278 | 1.00 | 187.77 | A | C |
| ATOM | 2799 | CB | PHE | B | 190 | −23.978 | 24.512 | 27.441 | 1.00 | 193.75 | A | C |
| ATOM | 2800 | CG | PHE | B | 190 | −23.222 | 23.696 | 26.429 | 1.00 | 239.12 | A | C |
| ATOM | 2801 | CD1 | PHE | B | 190 | −23.331 | 23.965 | 25.066 | 1.00 | 224.74 | A | C |
| ATOM | 2802 | CE1 | PHE | B | 190 | −22.631 | 23.209 | 24.143 | 1.00 | 228.04 | A | C |
| ATOM | 2803 | CZ | PHE | B | 190 | −21.819 | 22.163 | 24.571 | 1.00 | 210.14 | A | C |
| ATOM | 2804 | CE2 | PHE | B | 190 | −21.707 | 21.879 | 25.919 | 1.00 | 166.93 | A | C |
| ATOM | 2805 | CD2 | PHE | B | 190 | −22.411 | 22.639 | 26.836 | 1.00 | 237.31 | A | C |
| ATOM | 2806 | C | PHE | B | 190 | −24.810 | 26.745 | 28.168 | 1.00 | 214.64 | A | C |
| ATOM | 2807 | O | PHE | B | 190 | −25.949 | 26.971 | 27.775 | 1.00 | 248.51 | A | O |
| ATOM | 2808 | N | ILE | B | 191 | −24.361 | 27.141 | 29.356 | 1.00 | 269.76 | A | N |
| ATOM | 2809 | CA | ILE | B | 191 | −25.248 | 27.579 | 30.443 | 1.00 | 260.24 | A | C |
| ATOM | 2810 | CB | ILE | B | 191 | −24.925 | 26.783 | 31.726 | 1.00 | 270.35 | A | C |
| ATOM | 2811 | CG1 | ILE | B | 191 | −25.494 | 25.360 | 31.625 | 1.00 | 284.85 | A | C |
| ATOM | 2812 | CD1 | ILE | B | 191 | −25.154 | 24.448 | 32.795 | 1.00 | 325.78 | A | C |
| ATOM | 2813 | CG2 | ILE | B | 191 | −25.429 | 27.479 | 32.941 | 1.00 | 258.98 | A | C |
| ATOM | 2814 | C | ILE | B | 191 | −25.222 | 29.089 | 30.698 | 1.00 | 284.09 | A | C |
| ATOM | 2815 | O | ILE | B | 191 | −26.261 | 29.724 | 30.653 | 1.00 | 285.96 | A | O |
| ATOM | 2816 | N | GLN | B | 192 | −24.040 | 29.647 | 30.964 | 1.00 | 307.27 | A | N |
| ATOM | 2817 | CA | GLN | B | 192 | −23.872 | 31.091 | 31.167 | 1.00 | 318.44 | A | C |
| ATOM | 2818 | CB | GLN | B | 192 | −22.872 | 31.390 | 32.292 | 1.00 | 304.48 | A | C |
| ATOM | 2819 | CG | GLN | B | 192 | −23.202 | 30.774 | 33.652 | 1.00 | 330.86 | A | C |
| ATOM | 2820 | CD | GLN | B | 192 | −24.339 | 31.444 | 34.428 | 1.00 | 223.40 | A | C |
| ATOM | 2821 | OE1 | GLN | B | 192 | −24.637 | 32.632 | 34.236 | 1.00 | 280.73 | A | O |
| ATOM | 2822 | NE2 | GLN | B | 192 | −24.965 | 30.683 | 35.330 | 1.00 | 304.35 | A | N |
| ATOM | 2823 | C | GLN | B | 192 | −23.412 | 31.736 | 29.861 | 1.00 | 286.89 | A | C |
| ATOM | 2824 | O | GLN | B | 192 | −23.434 | 32.953 | 29.717 | 1.00 | 285.64 | A | O |
| ATOM | 2825 | N | HIS | B | 193 | −22.979 | 30.902 | 28.919 | 1.00 | 278.78 | A | N |
| ATOM | 2826 | CA | HIS | B | 193 | −22.862 | 31.302 | 27.519 | 1.00 | 271.32 | A | C |
| ATOM | 2827 | CB | HIS | B | 193 | −24.096 | 32.098 | 27.080 | 1.00 | 280.44 | A | C |
| ATOM | 2828 | CG | HIS | B | 193 | −25.401 | 31.369 | 27.321 | 1.00 | 285.26 | A | C |
| ATOM | 2829 | ND1 | HIS | B | 193 | −26.504 | 31.578 | 26.569 | 1.00 | 266.76 | A | N |
| ATOM | 2830 | CE1 | HIS | B | 193 | −27.499 | 30.784 | 27.008 | 1.00 | 257.12 | A | C |
| ATOM | 2831 | NE2 | HIS | B | 193 | −27.022 | 30.035 | 28.016 | 1.00 | 237.90 | A | N |
| ATOM | 2832 | CD2 | HIS | B | 193 | −25.730 | 30.361 | 28.224 | 1.00 | 278.49 | A | C |
| ATOM | 2833 | C | HIS | B | 193 | −21.593 | 32.013 | 27.173 | 1.00 | 269.22 | A | C |
| ATOM | 2834 | O | HIS | B | 193 | −20.923 | 31.625 | 26.221 | 1.00 | 293.33 | A | O |
| ATOM | 2835 | N | TRP | B | 194 | −21.256 | 33.073 | 27.902 | 1.00 | 267.27 | A | N |
| ATOM | 2836 | CA | TRP | B | 194 | −19.921 | 33.672 | 27.760 | 1.00 | 252.48 | A | C |
| ATOM | 2837 | CB | TRP | B | 194 | −19.952 | 35.075 | 27.156 | 1.00 | 253.78 | A | C |
| ATOM | 2838 | CG | TRP | B | 194 | −19.980 | 35.072 | 25.647 | 1.00 | 251.41 | A | C |
| ATOM | 2839 | CD1 | TRP | B | 194 | −21.096 | 35.187 | 24.827 | 1.00 | 270.66 | A | C |
| ATOM | 2840 | NE1 | TRP | B | 194 | −20.742 | 35.130 | 23.501 | 1.00 | 299.93 | A | N |
| ATOM | 2841 | CE2 | TRP | B | 194 | −19.408 | 34.982 | 23.358 | 1.00 | 297.95 | A | C |
| ATOM | 2842 | CD2 | TRP | B | 194 | −18.840 | 34.929 | 24.712 | 1.00 | 270.83 | A | C |
| ATOM | 2843 | CE3 | TRP | B | 194 | −17.468 | 34.775 | 24.852 | 1.00 | 257.49 | A | C |
| ATOM | 2844 | CZ3 | TRP | B | 194 | −16.680 | 34.692 | 23.686 | 1.00 | 248.90 | A | C |
| ATOM | 2845 | CH2 | TRP | B | 194 | −17.236 | 34.732 | 22.401 | 1.00 | 232.62 | A | C |
| ATOM | 2846 | CZ2 | TRP | B | 194 | −18.613 | 34.881 | 22.211 | 1.00 | 293.04 | A | C |
| ATOM | 2847 | C | TRP | B | 194 | −19.181 | 33.667 | 29.058 | 1.00 | 278.50 | A | C |
| ATOM | 2848 | O | TRP | B | 194 | −19.788 | 33.741 | 30.126 | 1.00 | 281.02 | A | O |
| ATOM | 2849 | N | VAL | B | 195 | −17.855 | 33.542 | 28.973 | 1.00 | 326.06 | A | N |
| ATOM | 2850 | CA | VAL | B | 195 | −16.981 | 33.646 | 30.138 | 1.00 | 289.24 | A | C |
| ATOM | 2851 | CB | VAL | B | 195 | −17.132 | 35.046 | 30.792 | 1.00 | 286.00 | A | C |
| ATOM | 2852 | CG1 | VAL | B | 195 | −16.153 | 35.244 | 31.948 | 1.00 | 292.20 | A | C |
| ATOM | 2853 | CG2 | VAL | B | 195 | −16.964 | 36.124 | 29.741 | 1.00 | 260.10 | A | C |
| ATOM | 2854 | C | VAL | B | 195 | −17.341 | 32.559 | 31.165 | 1.00 | 314.92 | A | C |
| ATOM | 2855 | O | VAL | B | 195 | −18.303 | 31.804 | 30.994 | 1.00 | 323.91 | A | O |
| ATOM | 2856 | OXT | VAL | B | 195 | −16.680 | 32.414 | 32.195 | 1.00 | 328.16 | | O |
| ATOM | 2857 | N | MET | C | 1 | −14.529 | 33.309 | −14.828 | 1.00 | 171.03 | A | N |
| ATOM | 2858 | CA | MET | C | 1 | −13.476 | 32.650 | −13.994 | 1.00 | 194.07 | A | C |
| ATOM | 2859 | CB | MET | C | 1 | −14.016 | 31.378 | −13.335 | 1.00 | 214.44 | A | C |
| ATOM | 2860 | CG | MET | C | 1 | −14.857 | 31.639 | −12.093 | 1.00 | 257.74 | A | C |
| ATOM | 2861 | SD | MET | C | 1 | −15.867 | 30.221 | −11.594 | 1.00 | 245.27 | A | S |
| ATOM | 2862 | CE | MET | C | 1 | −14.665 | 29.117 | −10.855 | 1.00 | 145.49 | A | C |
| ATOM | 2863 | C | MET | C | 1 | −12.228 | 32.345 | −14.811 | 1.00 | 201.51 | A | C |
| ATOM | 2864 | O | MET | C | 1 | −11.248 | 31.788 | −14.295 | 1.00 | 175.94 | A | O |
| ATOM | 2865 | N | LEU | C | 2 | −12.266 | 32.757 | −16.078 | 1.00 | 200.73 | A | N |
| ATOM | 2866 | CA | LEU | C | 2 | −11.179 | 32.521 | −17.026 | 1.00 | 186.98 | A | C |
| ATOM | 2867 | CB | LEU | C | 2 | −11.622 | 32.829 | −18.463 | 1.00 | 181.30 | A | C |
| ATOM | 2868 | CG | LEU | C | 2 | −12.717 | 31.917 | −19.031 | 1.00 | 207.66 | A | C |
| ATOM | 2869 | CD1 | LEU | C | 2 | −14.096 | 32.532 | −18.778 | 1.00 | 261.42 | A | C |
| ATOM | 2870 | CD2 | LEU | C | 2 | −12.475 | 31.637 | −20.518 | 1.00 | 194.98 | A | C |
| ATOM | 2871 | C | LEU | C | 2 | −9.933 | 33.313 | −16.665 | 1.00 | 187.10 | A | C |
| ATOM | 2872 | O | LEU | C | 2 | −8.820 | 32.897 | −16.974 | 1.00 | 162.17 | A | O |
| ATOM | 2873 | N | GLY | C | 3 | −10.131 | 34.454 | −16.009 | 1.00 | 191.75 | A | N |
| ATOM | 2874 | CA | GLY | C | 3 | −9.031 | 35.202 | −15.409 | 1.00 | 186.78 | A | C |
| ATOM | 2875 | C | GLY | C | 3 | −8.248 | 34.310 | −14.465 | 1.00 | 172.43 | A | C |
| ATOM | 2876 | O | GLY | C | 3 | −7.019 | 34.243 | −14.528 | 1.00 | 194.47 | A | O |
| ATOM | 2877 | N | LEU | C | 4 | −8.968 | 33.617 | −13.589 | 1.00 | 168.14 | A | N |
| ATOM | 2878 | CA | LEU | C | 4 | −8.370 | 32.635 | −12.705 | 1.00 | 153.00 | A | C |
| ATOM | 2879 | CB | LEU | C | 4 | −9.373 | 32.198 | −11.646 | 1.00 | 143.76 | A | C |
| ATOM | 2880 | CG | LEU | C | 4 | −9.019 | 32.528 | −10.209 | 1.00 | 148.96 | A | C |
| ATOM | 2881 | CD1 | LEU | C | 4 | −10.146 | 32.052 | −9.321 | 1.00 | 152.98 | A | C |
| ATOM | 2882 | CD2 | LEU | C | 4 | −7.724 | 31.834 | −9.821 | 1.00 | 152.21 | A | C |
| ATOM | 2883 | C | LEU | C | 4 | −7.897 | 31.417 | −13.467 | 1.00 | 143.70 | A | C |
| ATOM | 2884 | O | LEU | C | 4 | −6.699 | 31.151 | −13.539 | 1.00 | 133.97 | A | O |
| ATOM | 2885 | N | VAL | C | 5 | −8.850 | 30.684 | −14.033 | 1.00 | 157.99 | A | N |
| ATOM | 2886 | CA | VAL | C | 5 | −8.561 | 29.407 | −14.678 | 1.00 | 171.77 | A | C |
| ATOM | 2887 | CB | VAL | C | 5 | −9.789 | 28.822 | −15.427 | 1.00 | 172.17 | A | C |
| ATOM | 2888 | CG1 | VAL | C | 5 | −9.405 | 27.626 | −16.300 | 1.00 | 135.61 | A | C |
| ATOM | 2889 | CG2 | VAL | C | 5 | −10.884 | 28.434 | −14.447 | 1.00 | 177.00 | A | C |
| ATOM | 2890 | C | VAL | C | 5 | −7.395 | 29.543 | −15.641 | 1.00 | 159.74 | A | C |
| ATOM | 2891 | O | VAL | C | 5 | −6.443 | 28.775 | −15.558 | 1.00 | 141.00 | A | O |
| ATOM | 2892 | N | LEU | C | 6 | −7.469 | 30.535 | −16.528 | 1.00 | 157.34 | A | N |
| ATOM | 2893 | CA | LEU | C | 6 | −6.491 | 30.685 | −17.608 | 1.00 | 153.93 | A | C |
| ATOM | 2894 | CB | LEU | C | 6 | −7.032 | 31.535 | −18.759 | 1.00 | 149.54 | A | C |
| ATOM | 2895 | CG | LEU | C | 6 | −8.042 | 30.805 | −19.631 | 1.00 | 185.23 | A | C |
| ATOM | 2896 | CD1 | LEU | C | 6 | −8.433 | 31.654 | −20.837 | 1.00 | 181.37 | A | C |
| ATOM | 2897 | CD2 | LEU | C | 6 | −7.522 | 29.443 | −20.097 | 1.00 | 206.64 | A | C |
| ATOM | 2898 | C | LEU | C | 6 | −5.162 | 31.213 | −17.141 | 1.00 | 145.71 | A | C |
| ATOM | 2899 | O | LEU | C | 6 | −4.139 | 30.951 | −17.758 | 1.00 | 150.79 | A | O |
| ATOM | 2900 | N | LEU | C | 7 | −5.173 | 31.952 | −16.044 | 1.00 | 147.78 | A | N |
| ATOM | 2901 | CA | LEU | C | 7 | −3.927 | 32.338 | −15.420 | 1.00 | 145.78 | A | C |
| ATOM | 2902 | CB | LEU | C | 7 | −4.175 | 33.079 | −14.107 | 1.00 | 134.99 | A | C |
| ATOM | 2903 | CG | LEU | C | 7 | −2.920 | 33.309 | −13.276 | 1.00 | 130.48 | A | C |
| ATOM | 2904 | CD1 | LEU | C | 7 | −1.903 | 34.111 | −14.071 | 1.00 | 125.67 | A | C |
| ATOM | 2905 | CD2 | LEU | C | 7 | −3.261 | 33.994 | −11.970 | 1.00 | 142.02 | A | C |
| ATOM | 2906 | C | LEU | C | 7 | −3.067 | 31.102 | −15.169 | 1.00 | 144.15 | A | C |
| ATOM | 2907 | O | LEU | C | 7 | −1.871 | 31.099 | −15.457 | 1.00 | 161.80 | A | O |
| ATOM | 2908 | N | TYR | C | 8 | −3.687 | 30.047 | −14.646 | 1.00 | 142.23 | A | N |
| ATOM | 2909 | CA | TYR | C | 8 | −2.957 | 28.845 | −14.264 | 1.00 | 123.36 | A | C |
| ATOM | 2910 | CB | TYR | C | 8 | −3.641 | 28.172 | −13.089 | 1.00 | 113.11 | A | C |
| ATOM | 2911 | CG | TYR | C | 8 | −3.511 | 28.980 | −11.825 | 1.00 | 140.42 | A | C |
| ATOM | 2912 | CD1 | TYR | C | 8 | −2.393 | 28.838 | −10.998 | 1.00 | 138.79 | A | C |
| ATOM | 2913 | CE1 | TYR | C | 8 | −2.254 | 29.592 | −9.848 | 1.00 | 149.29 | A | C |
| ATOM | 2914 | CZ | TYR | C | 8 | −3.247 | 30.501 | −9.508 | 1.00 | 145.56 | A | C |
| ATOM | 2915 | OH | TYR | C | 8 | −3.129 | 31.248 | −8.360 | 1.00 | 152.49 | A | O |
| ATOM | 2916 | CE2 | TYR | C | 8 | −4.364 | 30.662 | −10.314 | 1.00 | 142.35 | A | C |
| ATOM | 2917 | CD2 | TYR | C | 8 | −4.487 | 29.910 | −11.468 | 1.00 | 139.02 | A | C |
| ATOM | 2918 | C | TYR | C | 8 | −2.766 | 27.891 | −15.437 | 1.00 | 141.25 | A | C |
| ATOM | 2919 | O | TYR | C | 8 | −1.744 | 27.208 | −15.513 | 1.00 | 150.98 | A | O |
| ATOM | 2920 | N | VAL | C | 9 | −3.732 | 27.868 | −16.358 | 1.00 | 123.03 | A | N |
| ATOM | 2921 | CA | VAL | C | 9 | −3.539 | 27.243 | −17.661 | 1.00 | 132.32 | A | C |
| ATOM | 2922 | CB | VAL | C | 9 | −4.738 | 27.465 | −18.603 | 1.00 | 148.05 | A | C |
| ATOM | 2923 | CG1 | VAL | C | 9 | −4.404 | 27.057 | −20.040 | 1.00 | 154.25 | A | C |
| ATOM | 2924 | CG2 | VAL | C | 9 | −5.933 | 26.677 | −18.104 | 1.00 | 147.74 | A | C |
| ATOM | 2925 | C | VAL | C | 9 | −2.266 | 27.772 | −18.312 | 1.00 | 146.49 | A | C |
| ATOM | 2926 | O | VAL | C | 9 | −1.596 | 27.064 | −19.080 | 1.00 | 165.00 | A | O |
| ATOM | 2927 | N | GLY | C | 10 | −1.934 | 29.018 | −17.990 | 1.00 | 132.06 | A | N |
| ATOM | 2928 | CA | GLY | C | 10 | −0.678 | 29.600 | −18.430 | 1.00 | 167.51 | A | C |
| ATOM | 2929 | C | GLY | C | 10 | 0.513 | 28.759 | −18.016 | 1.00 | 167.88 | A | C |
| ATOM | 2930 | O | GLY | C | 10 | 1.232 | 28.220 | −18.863 | 1.00 | 191.52 | A | O |
| ATOM | 2931 | N | ILE | C | 11 | 0.717 | 28.644 | −16.710 | 1.00 | 163.10 | A | N |
| ATOM | 2932 | CA | ILE | C | 11 | 1.898 | 27.965 | −16.182 | 1.00 | 175.89 | A | C |
| ATOM | 2933 | CB | ILE | C | 11 | 1.991 | 28.079 | −14.654 | 1.00 | 165.37 | A | C |
| ATOM | 2934 | CG1 | ILE | C | 11 | 1.331 | 29.366 | −14.184 | 1.00 | 189.82 | A | C |
| ATOM | 2935 | CD1 | ILE | C | 11 | 0.686 | 29.260 | −12.822 | 1.00 | 217.26 | A | C |
| ATOM | 2936 | CG2 | ILE | C | 11 | 3.449 | 28.006 | −14.212 | 1.00 | 176.36 | A | C |
| ATOM | 2937 | C | ILE | C | 11 | 1.914 | 26.493 | −16.556 | 1.00 | 176.08 | A | C |
| ATOM | 2938 | O | ILE | C | 11 | 2.915 | 25.994 | −17.067 | 1.00 | 184.39 | A | O |
| ATOM | 2939 | N | VAL | C | 12 | 0.800 | 25.809 | −16.309 | 1.00 | 169.49 | A | N |
| ATOM | 2940 | CA | VAL | C | 12 | 0.708 | 24.374 | −16.567 | 1.00 | 168.71 | A | C |
| ATOM | 2941 | CB | VAL | C | 12 | −0.688 | 23.813 | −16.215 | 1.00 | 149.64 | A | C |
| ATOM | 2942 | CG1 | VAL | C | 12 | −1.736 | 24.346 | −17.174 | 1.00 | 164.34 | A | C |
| ATOM | 2943 | CG2 | VAL | C | 12 | −0.682 | 22.292 | −16.235 | 1.00 | 149.77 | A | C |
| ATOM | 2944 | C | VAL | C | 12 | 1.056 | 24.045 | −18.014 | 1.00 | 148.01 | A | C |
| ATOM | 2945 | O | VAL | C | 12 | 1.660 | 23.006 | −18.277 | 1.00 | 145.68 | A | O |
| ATOM | 2946 | N | LEU | C | 13 | 0.675 | 24.931 | −18.935 | 1.00 | 151.47 | A | N |
| ATOM | 2947 | CA | LEU | C | 13 | 1.039 | 24.763 | −20.329 | 1.00 | 142.69 | A | C |
| ATOM | 2948 | CB | LEU | C | 13 | 0.188 | 25.648 | −21.243 | 1.00 | 149.24 | A | C |
| ATOM | 2949 | CG | LEU | C | 13 | −0.927 | 24.901 | −21.972 | 1.00 | 145.93 | A | C |
| ATOM | 2950 | CD1 | LEU | C | 13 | −1.717 | 25.881 | −22.811 | 1.00 | 165.95 | A | C |
| ATOM | 2951 | CD2 | LEU | C | 13 | −0.401 | 23.728 | −22.801 | 1.00 | 168.22 | A | C |
| ATOM | 2952 | C | LEU | C | 13 | 2.515 | 25.028 | −20.569 | 1.00 | 171.71 | A | C |
| ATOM | 2953 | O | LEU | C | 13 | 3.174 | 24.292 | −21.304 | 1.00 | 172.49 | A | O |
| ATOM | 2954 | N | ILE | C | 14 | 3.026 | 26.088 | −19.948 | 1.00 | 185.60 | A | N |
| ATOM | 2955 | CA | ILE | C | 14 | 4.462 | 26.363 | −19.945 | 1.00 | 206.33 | A | C |
| ATOM | 2956 | CB | ILE | C | 14 | 4.787 | 27.689 | −19.212 | 1.00 | 196.93 | A | C |
| ATOM | 2957 | CG1 | ILE | C | 14 | 4.635 | 28.871 | −20.172 | 1.00 | 214.48 | A | C |
| ATOM | 2958 | CD1 | ILE | C | 14 | 4.856 | 30.243 | −19.557 | 1.00 | 251.87 | A | C |
| ATOM | 2959 | CG2 | ILE | C | 14 | 6.199 | 27.673 | −18.636 | 1.00 | 149.51 | A | C |
| ATOM | 2960 | C | ILE | C | 14 | 5.226 | 25.190 | −19.314 | 1.00 | 174.60 | A | C |
| ATOM | 2961 | O | ILE | C | 14 | 6.236 | 24.729 | −19.858 | 1.00 | 188.60 | A | O |
| ATOM | 2962 | N | SER | C | 15 | 4.724 | 24.703 | −18.181 | 1.00 | 147.13 | A | N |
| ATOM | 2963 | CA | SER | C | 15 | 5.380 | 23.650 | −17.433 | 1.00 | 128.50 | A | C |
| ATOM | 2964 | CB | SER | C | 15 | 4.544 | 23.299 | −16.203 | 1.00 | 144.15 | A | C |
| ATOM | 2965 | OG | SER | C | 15 | 4.892 | 22.024 | −15.730 | 1.00 | 147.16 | A | O |
| ATOM | 2966 | C | SER | C | 15 | 5.614 | 22.432 | −18.335 | 1.00 | 143.15 | A | C |
| ATOM | 2967 | O | SER | C | 15 | 6.745 | 21.977 | −18.476 | 1.00 | 131.79 | A | O |
| ATOM | 2968 | N | ASN | C | 16 | 4.550 | 21.935 | −18.962 | 1.00 | 157.62 | A | N |
| ATOM | 2969 | CA | ASN | C | 16 | 4.632 | 20.803 | −19.888 | 1.00 | 170.88 | A | C |
| ATOM | 2970 | CB | ASN | C | 16 | 3.247 | 20.461 | −20.438 | 1.00 | 165.75 | A | C |
| ATOM | 2971 | CG | ASN | C | 16 | 2.391 | 19.695 | −19.434 | 1.00 | 202.50 | A | C |
| ATOM | 2972 | OD1 | ASN | C | 16 | 2.878 | 18.826 | −18.706 | 1.00 | 179.34 | A | O |
| ATOM | 2973 | ND2 | ASN | C | 16 | 1.091 | 20.016 | −19.413 | 1.00 | 167.65 | A | N |
| ATOM | 2974 | C | ASN | C | 16 | 5.621 | 20.988 | −21.037 | 1.00 | 171.10 | A | C |
| ATOM | 2975 | O | ASN | C | 16 | 6.319 | 20.051 | −21.441 | 1.00 | 160.97 | A | O |
| ATOM | 2976 | N | GLY | C | 17 | 5.685 | 22.206 | −21.564 | 1.00 | 150.56 | A | N |
| ATOM | 2977 | CA | GLY | C | 17 | 6.688 | 22.551 | −22.569 | 1.00 | 181.68 | A | C |
| ATOM | 2978 | C | GLY | C | 17 | 8.104 | 22.335 | −22.061 | 1.00 | 189.96 | A | C |
| ATOM | 2979 | O | GLY | C | 17 | 8.873 | 21.563 | −22.651 | 1.00 | 172.48 | A | O |
| ATOM | 2980 | N | ILE | C | 18 | 8.435 | 23.007 | −20.957 | 1.00 | 184.54 | A | N |
| ATOM | 2981 | CA | ILE | C | 18 | 9.765 | 22.938 | −20.346 | 1.00 | 175.53 | A | C |
| ATOM | 2982 | CB | ILE | C | 18 | 9.871 | 23.822 | −19.088 | 1.00 | 163.58 | A | C |
| ATOM | 2983 | CG1 | ILE | C | 18 | 9.694 | 25.291 | −19.496 | 1.00 | 164.75 | A | C |
| ATOM | 2984 | CD1 | ILE | C | 18 | 9.550 | 26.258 | −18.342 | 1.00 | 151.17 | A | C |
| ATOM | 2985 | CG2 | ILE | C | 18 | 11.219 | 23.646 | −18.407 | 1.00 | 146.51 | A | C |
| ATOM | 2986 | C | ILE | C | 18 | 10.145 | 21.509 | −20.035 | 1.00 | 175.31 | A | C |
| ATOM | 2987 | O | ILE | C | 18 | 11.278 | 21.112 | −20.254 | 1.00 | 176.85 | A | O |
| ATOM | 2988 | N | CYS | C | 19 | 9.190 | 20.733 | −19.543 | 1.00 | 166.81 | A | N |
| ATOM | 2989 | CA | CYS | C | 19 | 9.465 | 19.377 | −19.111 | 1.00 | 158.15 | A | C |
| ATOM | 2990 | CB | CYS | C | 19 | 8.358 | 18.886 | −18.188 | 1.00 | 156.16 | A | C |
| ATOM | 2991 | SG | CYS | C | 19 | 8.352 | 19.837 | −16.651 | 1.00 | 155.25 | A | S |
| ATOM | 2992 | C | CYS | C | 19 | 9.686 | 18.412 | −20.247 | 1.00 | 148.00 | A | C |
| ATOM | 2993 | O | CYS | C | 19 | 10.184 | 17.326 | −20.026 | 1.00 | 186.33 | A | O |
| ATOM | 2994 | N | GLY | C | 20 | 9.297 | 18.794 | −21.458 | 1.00 | 161.03 | A | N |
| ATOM | 2995 | CA | GLY | C | 20 | 9.643 | 18.037 | −22.669 | 1.00 | 207.76 | A | C |
| ATOM | 2996 | C | GLY | C | 20 | 11.049 | 18.405 | −23.080 | 1.00 | 192.24 | A | C |
| ATOM | 2997 | O | GLY | C | 20 | 11.711 | 17.657 | −23.824 | 1.00 | 210.88 | A | O |
| ATOM | 2998 | N | LEU | C | 21 | 11.513 | 19.549 | −22.569 | 1.00 | 196.62 | A | N |
| ATOM | 2999 | CA | LEU | C | 21 | 12.794 | 20.136 | −22.970 | 1.00 | 195.22 | A | C |
| ATOM | 3000 | CB | LEU | C | 21 | 12.699 | 21.648 | −23.062 | 1.00 | 200.37 | A | C |
| ATOM | 3001 | CG | LEU | C | 21 | 11.704 | 22.206 | −24.071 | 1.00 | 223.23 | A | C |
| ATOM | 3002 | CD1 | LEU | C | 21 | 11.658 | 23.703 | −23.880 | 1.00 | 228.72 | A | C |
| ATOM | 3003 | CD2 | LEU | C | 21 | 12.071 | 21.835 | −25.494 | 1.00 | 253.03 | A | C |
| ATOM | 3004 | C | LEU | C | 21 | 13.877 | 19.749 | −21.985 | 1.00 | 175.75 | A | C |
| ATOM | 3005 | O | LEU | C | 21 | 14.750 | 18.967 | −22.347 | 1.00 | 209.61 | A | O |
| ATOM | 3006 | N | THR | C | 22 | 13.825 | 20.280 | −20.755 | 1.00 | 171.88 | A | N |
| ATOM | 3007 | CA | THR | C | 22 | 14.762 | 19.845 | −19.694 | 1.00 | 211.67 | A | C |
| ATOM | 3008 | CB | THR | C | 22 | 14.725 | 20.712 | −18.388 | 1.00 | 190.98 | A | C |
| ATOM | 3009 | OG1 | THR | C | 22 | 13.383 | 21.072 | −18.047 | 1.00 | 180.82 | A | O |
| ATOM | 3010 | CG2 | THR | C | 22 | 15.547 | 21.979 | −18.525 | 1.00 | 167.80 | A | C |
| ATOM | 3011 | C | THR | C | 22 | 14.599 | 18.348 | −19.368 | 1.00 | 176.69 | A | C |
| ATOM | 3012 | O | THR | C | 22 | 15.515 | 17.720 | −18.824 | 1.00 | 215.11 | A | O |
| ATOM | 3013 | N | LYS | C | 23 | 13.419 | 17.803 | −19.690 | 1.00 | 197.74 | A | N |
| ATOM | 3014 | CA | LYS | C | 23 | 13.062 | 16.373 | −19.534 | 1.00 | 190.93 | A | C |
| ATOM | 3015 | CB | LYS | C | 23 | 13.664 | 15.517 | −20.638 | 1.00 | 214.17 | A | C |
| ATOM | 3016 | CG | LYS | C | 23 | 12.709 | 15.472 | −21.813 | 1.00 | 218.83 | A | C |
| ATOM | 3017 | CD | LYS | C | 23 | 13.232 | 14.537 | −22.896 | 1.00 | 238.49 | A | C |
| ATOM | 3018 | CE | LYS | C | 23 | 13.991 | 15.312 | −23.980 | 1.00 | 237.40 | A | C |
| ATOM | 3019 | NZ | LYS | C | 23 | 13.986 | 14.532 | −25.252 | 1.00 | 308.76 | A | N |
| ATOM | 3020 | C | LYS | C | 23 | 13.156 | 15.724 | −18.153 | 1.00 | 177.07 | A | C |
| ATOM | 3021 | O | LYS | C | 23 | 14.084 | 14.960 | −17.829 | 1.00 | 171.63 | A | O |
| ATOM | 3022 | N | VAL | C | 24 | 12.133 | 16.015 | −17.366 | 1.00 | 159.23 | A | N |
| ATOM | 3023 | CA | VAL | C | 24 | 12.182 | 15.695 | −15.998 | 1.00 | 161.35 | A | C |
| ATOM | 3024 | CB | VAL | C | 24 | 11.935 | 16.971 | −15.149 | 1.00 | 149.80 | A | C |
| ATOM | 3025 | CG1 | VAL | C | 24 | 10.735 | 17.730 | −15.652 | 1.00 | 139.12 | A | C |
| ATOM | 3026 | CG2 | VAL | C | 24 | 11.799 | 16.655 | −13.667 | 1.00 | 179.69 | A | C |
| ATOM | 3027 | C | VAL | C | 24 | 11.218 | 14.545 | −15.679 | 1.00 | 166.22 | A | C |
| ATOM | 3028 | O | VAL | C | 24 | 10.172 | 14.399 | −16.317 | 1.00 | 167.88 | A | O |
| ATOM | 3029 | N | ASP | C | 25 | 11.693 | 13.670 | −14.794 | 1.00 | 156.19 | A | N |
| ATOM | 3030 | CA | ASP | C | 25 | 10.907 | 12.886 | −13.854 | 1.00 | 162.54 | A | C |
| ATOM | 3031 | CB | ASP | C | 25 | 11.403 | 13.294 | −12.468 | 1.00 | 144.01 | A | C |
| ATOM | 3032 | CG | ASP | C | 25 | 10.660 | 12.602 | −11.343 | 1.00 | 157.58 | A | C |
| ATOM | 3033 | OD1 | ASP | C | 25 | 10.873 | 11.392 | −11.131 | 1.00 | 200.18 | A | O |
| ATOM | 3034 | OD2 | ASP | C | 25 | 9.873 | 13.280 | −10.655 | 1.00 | 164.76 | A | O |
| ATOM | 3035 | C | ASP | C | 25 | 9.375 | 13.060 | −13.965 | 1.00 | 154.79 | A | C |
| ATOM | 3036 | O | ASP | C | 25 | 8.810 | 13.999 | −13.396 | 1.00 | 151.19 | A | O |
| ATOM | 3037 | N | PRO | C | 26 | 8.696 | 12.136 | −14.674 | 1.00 | 136.61 | A | N |
| ATOM | 3038 | CA | PRO | C | 26 | 7.282 | 12.306 | −15.024 | 1.00 | 125.21 | A | C |
| ATOM | 3039 | CB | PRO | C | 26 | 6.926 | 10.990 | −15.704 | 1.00 | 118.21 | A | C |
| ATOM | 3040 | CG | PRO | C | 26 | 8.221 | 10.500 | −16.228 | 1.00 | 142.61 | A | C |
| ATOM | 3041 | CD | PRO | C | 26 | 9.184 | 10.826 | −15.118 | 1.00 | 147.28 | A | C |
| ATOM | 3042 | C | PRO | C | 26 | 6.383 | 12.513 | −13.826 | 1.00 | 118.88 | A | C |
| ATOM | 3043 | O | PRO | C | 26 | 5.329 | 13.135 | −13.958 | 1.00 | 122.61 | A | O |
| ATOM | 3044 | N | LYS | C | 27 | 6.787 | 11.992 | −12.672 | 1.00 | 100.72 | A | N |
| ATOM | 3045 | CA | LYS | C | 27 | 6.037 | 12.225 | −11.471 | 1.00 | 99.20 | A | C |
| ATOM | 3046 | CB | LYS | C | 27 | 6.674 | 11.494 | −10.307 | 1.00 | 132.67 | A | C |
| ATOM | 3047 | CG | LYS | C | 27 | 6.917 | 10.005 | −10.510 | 1.00 | 148.69 | A | C |
| ATOM | 3048 | CD | LYS | C | 27 | 5.756 | 9.164 | −9.999 | 1.00 | 113.25 | A | C |
| ATOM | 3049 | CE | LYS | C | 27 | 6.232 | 7.781 | −9.589 | 1.00 | 146.39 | A | C |
| ATOM | 3050 | NZ | LYS | C | 27 | 5.128 | 6.919 | −9.040 | 1.00 | 170.96 | A | N |
| ATOM | 3051 | C | LYS | C | 27 | 6.004 | 13.724 | −11.144 | 1.00 | 130.65 | A | C |
| ATOM | 3052 | O | LYS | C | 27 | 4.982 | 14.228 | −10.685 | 1.00 | 141.29 | A | O |
| ATOM | 3053 | N | SER | C | 28 | 7.114 | 14.430 | −11.380 | 1.00 | 149.37 | A | N |
| ATOM | 3054 | CA | SER | C | 28 | 7.239 | 15.843 | −10.984 | 1.00 | 137.71 | A | C |
| ATOM | 3055 | CB | SER | C | 28 | 8.696 | 16.314 | −11.003 | 1.00 | 172.87 | A | C |
| ATOM | 3056 | OG | SER | C | 28 | 9.504 | 15.502 | −10.166 | 1.00 | 133.05 | A | O |
| ATOM | 3057 | C | SER | C | 28 | 6.417 | 16.742 | −11.861 | 1.00 | 123.17 | A | C |
| ATOM | 3058 | O | SER | C | 28 | 5.692 | 17.603 | −11.338 | 1.00 | 135.42 | A | O |
| ATOM | 3059 | N | THR | C | 29 | 6.521 | 16.535 | −13.180 | 1.00 | 123.43 | A | N |
| ATOM | 3060 | CA | THR | C | 29 | 5.713 | 17.292 | −14.130 | 1.00 | 120.83 | A | C |
| ATOM | 3061 | CB | THR | C | 29 | 5.838 | 16.759 | −15.555 | 1.00 | 117.13 | A | C |
| ATOM | 3062 | OG1 | THR | C | 29 | 5.059 | 15.578 | −15.686 | 1.00 | 151.49 | A | O |
| ATOM | 3063 | CG2 | THR | C | 29 | 7.255 | 16.423 | −15.922 | 1.00 | 132.88 | A | C |
| ATOM | 3064 | C | THR | C | 29 | 4.227 | 17.212 | −13.749 | 1.00 | 130.18 | A | C |
| ATOM | 3065 | O | THR | C | 29 | 3.517 | 18.226 | −13.759 | 1.00 | 142.18 | A | O |
| ATOM | 3066 | N | ALA | C | 30 | 3.780 | 16.006 | −13.400 | 1.00 | 122.21 | A | N |
| ATOM | 3067 | CA | ALA | C | 30 | 2.365 | 15.730 | −13.179 | 1.00 | 126.99 | A | C |
| ATOM | 3068 | CB | ALA | C | 30 | 2.154 | 14.261 | −12.845 | 1.00 | 109.31 | A | C |
| ATOM | 3069 | C | ALA | C | 30 | 1.785 | 16.629 | −12.099 | 1.00 | 126.21 | A | C |
| ATOM | 3070 | O | ALA | C | 30 | 0.636 | 17.078 | −12.208 | 1.00 | 140.11 | A | O |
| ATOM | 3071 | N | VAL | C | 31 | 2.588 | 16.918 | −11.078 | 1.00 | 104.36 | A | N |
| ATOM | 3072 | CA | VAL | C | 31 | 2.131 | 17.715 | −9.950 | 1.00 | 103.02 | A | C |
| ATOM | 3073 | CB | VAL | C | 31 | 3.299 | 18.218 | −9.118 | 1.00 | 96.09 | A | C |
| ATOM | 3074 | CG1 | VAL | C | 31 | 2.791 | 19.111 | −8.004 | 1.00 | 107.76 | A | C |
| ATOM | 3075 | CG2 | VAL | C | 31 | 4.094 | 17.054 | −8.571 | 1.00 | 109.80 | A | C |
| ATOM | 3076 | C | VAL | C | 31 | 1.343 | 18.925 | −10.417 | 1.00 | 114.98 | A | C |
| ATOM | 3077 | O | VAL | C | 31 | 0.207 | 19.159 | −9.985 | 1.00 | 119.59 | A | O |
| ATOM | 3078 | N | MET | C | 32 | 1.960 | 19.690 | −11.308 | 1.00 | 117.90 | A | N |
| ATOM | 3079 | CA | MET | C | 32 | 1.336 | 20.889 | −11.794 | 1.00 | 131.59 | A | C |
| ATOM | 3080 | CB | MET | C | 32 | 2.288 | 21.652 | −12.716 | 1.00 | 163.85 | A | C |
| ATOM | 3081 | CG | MET | C | 32 | 1.748 | 23.000 | −13.172 | 1.00 | 160.06 | A | C |
| ATOM | 3082 | SD | MET | C | 32 | 0.888 | 23.961 | −11.883 | 1.00 | 148.96 | A | S |
| ATOM | 3083 | CE | MET | C | 32 | 2.243 | 24.582 | −10.909 | 1.00 | 134.35 | A | C |
| ATOM | 3084 | C | MET | C | 32 | 0.029 | 20.541 | −12.488 | 1.00 | 117.05 | A | C |
| ATOM | 3085 | O | MET | C | 32 | −1.025 | 21.104 | −12.160 | 1.00 | 107.94 | A | O |
| ATOM | 3086 | N | ASN | C | 33 | 0.100 | 19.574 | −13.402 | 1.00 | 114.35 | A | N |
| ATOM | 3087 | CA | ASN | C | 33 | −1.072 | 19.121 | −14.148 | 1.00 | 117.75 | A | C |
| ATOM | 3088 | CB | ASN | C | 33 | −0.702 | 17.956 | −15.045 | 1.00 | 110.52 | A | C |
| ATOM | 3089 | CG | ASN | C | 33 | 0.327 | 18.337 | −16.082 | 1.00 | 128.49 | A | C |
| ATOM | 3090 | OD1 | ASN | C | 33 | 0.013 | 18.952 | −17.084 | 1.00 | 146.67 | A | O |
| ATOM | 3091 | ND2 | ASN | C | 33 | 1.565 | 17.970 | −15.845 | 1.00 | 133.26 | A | N |
| ATOM | 3092 | C | ASN | C | 33 | −2.230 | 18.742 | −13.256 | 1.00 | 113.14 | A | C |
| ATOM | 3093 | O | ASN | C | 33 | −3.391 | 18.921 | −13.620 | 1.00 | 102.80 | A | O |
| ATOM | 3094 | N | PHE | C | 34 | −1.897 | 18.233 | −12.078 | 1.00 | 115.43 | A | N |
| ATOM | 3095 | CA | PHE | C | 34 | −2.889 | 17.938 | −11.056 | 1.00 | 111.17 | A | C |
| ATOM | 3096 | CB | PHE | C | 34 | −2.253 | 17.153 | −9.936 | 1.00 | 102.10 | A | C |
| ATOM | 3097 | CG | PHE | C | 34 | −2.254 | 15.679 | −10.149 | 1.00 | 113.09 | A | C |
| ATOM | 3098 | CD1 | PHE | C | 34 | −3.444 | 14.968 | −10.158 | 1.00 | 109.45 | A | C |
| ATOM | 3099 | CE1 | PHE | C | 34 | −3.440 | 13.599 | −10.335 | 1.00 | 108.92 | A | C |
| ATOM | 3100 | CZ | PHE | C | 34 | −2.246 | 12.925 | −10.490 | 1.00 | 105.77 | A | C |
| ATOM | 3101 | CE2 | PHE | C | 34 | −1.049 | 13.622 | −10.477 | 1.00 | 112.51 | A | C |
| ATOM | 3102 | CD2 | PHE | C | 34 | −1.059 | 14.990 | −10.300 | 1.00 | 108.14 | A | C |
| ATOM | 3103 | C | PHE | C | 34 | −3.520 | 19.199 | −10.492 | 1.00 | 114.04 | A | C |
| ATOM | 3104 | O | PHE | C | 34 | −4.737 | 19.316 | −10.450 | 1.00 | 114.02 | A | O |
| ATOM | 3105 | N | PHE | C | 35 | −2.691 | 20.140 | −10.053 | 1.00 | 119.91 | A | N |
| ATOM | 3106 | CA | PHE | C | 35 | −3.191 | 21.372 | −9.473 | 1.00 | 124.34 | A | C |
| ATOM | 3107 | CB | PHE | C | 35 | −2.060 | 22.359 | −9.232 | 1.00 | 129.04 | A | C |
| ATOM | 3108 | CG | PHE | C | 35 | −1.224 | 22.017 | −8.062 | 1.00 | 117.86 | A | C |
| ATOM | 3109 | CD1 | PHE | C | 35 | −1.794 | 21.410 | −6.948 | 1.00 | 126.19 | A | C |
| ATOM | 3110 | CE1 | PHE | C | 35 | −1.024 | 21.092 | −5.847 | 1.00 | 131.91 | A | C |
| ATOM | 3111 | CZ | PHE | C | 35 | 0.324 | 21.387 | −5.839 | 1.00 | 124.71 | A | C |
| ATOM | 3112 | CE2 | PHE | C | 35 | 0.900 | 21.996 | −6.937 | 1.00 | 125.74 | A | C |
| ATOM | 3113 | CD2 | PHE | C | 35 | 0.126 | 22.319 | −8.047 | 1.00 | 128.61 | A | C |
| ATOM | 3114 | C | PHE | C | 35 | −4.201 | 22.012 | −10.372 | 1.00 | 116.21 | A | C |
| ATOM | 3115 | O | PHE | C | 35 | −5.306 | 22.346 | −9.940 | 1.00 | 107.16 | A | O |
| ATOM | 3116 | N | VAL | C | 36 | −3.801 | 22.169 | −11.628 | 1.00 | 106.10 | A | N |
| ATOM | 3117 | CA | VAL | C | 36 | −4.587 | 22.912 | −12.581 | 1.00 | 109.56 | A | C |
| ATOM | 3118 | CB | VAL | C | 36 | −3.752 | 23.297 | −13.775 | 1.00 | 114.58 | A | C |
| ATOM | 3119 | CG1 | VAL | C | 36 | −4.642 | 23.785 | −14.910 | 1.00 | 125.23 | A | C |
| ATOM | 3120 | CG2 | VAL | C | 36 | −2.757 | 24.365 | −13.350 | 1.00 | 142.80 | A | C |
| ATOM | 3121 | C | VAL | C | 36 | −5.829 | 22.148 | −13.006 | 1.00 | 126.78 | A | C |
| ATOM | 3122 | O | VAL | C | 36 | −6.943 | 22.627 | −12.807 | 1.00 | 125.35 | A | O |
| ATOM | 3123 | N | GLY | C | 37 | −5.642 | 20.963 | −13.581 | 1.00 | 112.35 | A | N |
| ATOM | 3124 | CA | GLY | C | 37 | −6.760 | 20.074 | −13.857 | 1.00 | 114.29 | A | C |
| ATOM | 3125 | C | GLY | C | 37 | −7.769 | 20.086 | −12.720 | 1.00 | 125.39 | A | C |
| ATOM | 3126 | O | GLY | C | 37 | −8.967 | 20.293 | −12.941 | 1.00 | 128.01 | A | O |
| ATOM | 3127 | N | GLY | C | 38 | −7.278 | 19.880 | −11.500 | 1.00 | 124.87 | A | N |
| ATOM | 3128 | CA | GLY | C | 38 | −8.111 | 19.916 | −10.305 | 1.00 | 116.10 | A | C |
| ATOM | 3129 | C | GLY | C | 38 | −8.839 | 21.232 | −10.196 | 1.00 | 125.79 | A | C |
| ATOM | 3130 | O | GLY | C | 38 | −10.047 | 21.264 | −10.025 | 1.00 | 131.65 | A | O |
| ATOM | 3131 | N | LEU | C | 39 | −8.103 | 22.322 | −10.323 | 1.00 | 136.51 | A | N |
| ATOM | 3132 | CA | LEU | C | 39 | −8.711 | 23.647 | −10.272 | 1.00 | 139.26 | A | C |
| ATOM | 3133 | CB | LEU | C | 39 | −7.656 | 24.723 | −10.507 | 1.00 | 125.75 | A | C |
| ATOM | 3134 | CG | LEU | C | 39 | −8.152 | 26.158 | −10.374 | 1.00 | 121.94 | A | C |
| ATOM | 3135 | CD1 | LEU | C | 39 | −8.787 | 26.413 | −9.007 | 1.00 | 121.44 | A | C |
| ATOM | 3136 | CD2 | LEU | C | 39 | −7.011 | 27.125 | −10.653 | 1.00 | 134.33 | A | C |
| ATOM | 3137 | C | LEU | C | 39 | −9.839 | 23.805 | −11.288 | 1.00 | 124.11 | A | C |
| ATOM | 3138 | O | LEU | C | 39 | −10.942 | 24.179 | −10.929 | 1.00 | 130.52 | A | O |
| ATOM | 3139 | N | SER | C | 40 | −9.567 | 23.503 | −12.548 | 1.00 | 121.32 | A | N |
| ATOM | 3140 | CA | SER | C | 40 | −10.575 | 23.613 | −13.605 | 1.00 | 123.24 | A | C |
| ATOM | 3141 | CB | SER | C | 40 | −10.105 | 22.933 | −14.885 | 1.00 | 129.04 | A | C |
| ATOM | 3142 | OG | SER | C | 40 | −8.694 | 22.953 | −14.983 | 1.00 | 144.12 | A | O |
| ATOM | 3143 | C | SER | C | 40 | −11.859 | 22.941 | −13.186 | 1.00 | 132.34 | A | C |
| ATOM | 3144 | O | SER | C | 40 | −12.935 | 23.514 | −13.285 | 1.00 | 146.36 | A | O |
| ATOM | 3145 | N | ILE | C | 41 | −11.731 | 21.721 | −12.695 | 1.00 | 129.50 | A | N |
| ATOM | 3146 | CA | ILE | C | 41 | −12.879 | 20.859 | −12.488 | 1.00 | 140.65 | A | C |
| ATOM | 3147 | CB | ILE | C | 41 | −12.419 | 19.403 | −12.330 | 1.00 | 138.48 | A | C |
| ATOM | 3148 | CG1 | ILE | C | 41 | −11.891 | 18.918 | −13.679 | 1.00 | 128.52 | A | C |
| ATOM | 3149 | CD1 | ILE | C | 41 | −11.005 | 17.705 | −13.566 | 1.00 | 162.86 | A | C |
| ATOM | 3150 | CG2 | ILE | C | 41 | −13.534 | 18.503 | −11.798 | 1.00 | 137.24 | A | C |
| ATOM | 3151 | C | ILE | C | 41 | −13.718 | 21.339 | −11.321 | 1.00 | 131.51 | A | C |
| ATOM | 3152 | O | ILE | C | 41 | −14.941 | 21.317 | −11.384 | 1.00 | 140.66 | A | O |
| ATOM | 3153 | N | VAL | C | 42 | −13.054 | 21.798 | −10.273 | 1.00 | 107.36 | A | N |
| ATOM | 3154 | CA | VAL | C | 42 | −13.749 | 22.455 | −9.192 | 1.00 | 113.54 | A | C |
| ATOM | 3155 | CB | VAL | C | 42 | −12.786 | 22.990 | −8.118 | 1.00 | 131.30 | A | C |
| ATOM | 3156 | CG1 | VAL | C | 42 | −13.573 | 23.450 | −6.900 | 1.00 | 126.79 | A | C |
| ATOM | 3157 | CG2 | VAL | C | 42 | −11.771 | 21.931 | −7.710 | 1.00 | 154.58 | A | C |
| ATOM | 3158 | C | VAL | C | 42 | −14.548 | 23.630 | −9.736 | 1.00 | 127.13 | A | C |
| ATOM | 3159 | O | VAL | C | 42 | −15.759 | 23.729 | −9.519 | 1.00 | 131.54 | A | O |
| ATOM | 3160 | N | CYS | C | 43 | −13.864 | 24.514 | −10.453 | 1.00 | 114.06 | A | N |
| ATOM | 3161 | CA | CYS | C | 43 | −14.477 | 25.733 | −10.940 | 1.00 | 127.77 | A | C |
| ATOM | 3162 | CB | CYS | C | 43 | −13.483 | 26.511 | −11.788 | 1.00 | 141.19 | A | C |
| ATOM | 3163 | SG | CYS | C | 43 | −12.064 | 27.096 | −10.839 | 1.00 | 152.70 | A | S |
| ATOM | 3164 | C | CYS | C | 43 | −15.729 | 25.429 | −11.735 | 1.00 | 132.39 | A | C |
| ATOM | 3165 | O | CYS | C | 43 | −16.823 | 25.868 | −11.389 | 1.00 | 135.12 | A | O |
| ATOM | 3166 | N | ASN | C | 44 | −15.570 | 24.623 | −12.772 | 1.00 | 123.27 | A | N |
| ATOM | 3167 | CA | ASN | C | 44 | −16.669 | 24.309 | −13.647 | 1.00 | 129.58 | A | C |
| ATOM | 3168 | CB | ASN | C | 44 | −16.195 | 23.457 | −14.818 | 1.00 | 144.19 | A | C |
| ATOM | 3169 | CG | ASN | C | 44 | −15.223 | 24.194 | −15.728 | 1.00 | 144.72 | A | C |
| ATOM | 3170 | OD1 | ASN | C | 44 | −14.456 | 23.579 | −16.449 | 1.00 | 125.93 | A | O |
| ATOM | 3171 | ND2 | ASN | C | 44 | −15.250 | 25.519 | −15.689 | 1.00 | 166.45 | A | N |
| ATOM | 3172 | C | ASN | C | 44 | −17.811 | 23.644 | −12.905 | 1.00 | 123.05 | A | C |
| ATOM | 3173 | O | ASN | C | 44 | −18.934 | 23.681 | −13.359 | 1.00 | 132.83 | A | O |
| ATOM | 3174 | N | VAL | C | 45 | −17.526 | 23.073 | −11.749 | 1.00 | 109.67 | A | N |
| ATOM | 3175 | CA | VAL | C | 45 | −18.549 | 22.426 | −10.951 | 1.00 | 124.67 | A | C |
| ATOM | 3176 | CB | VAL | C | 45 | −17.943 | 21.386 | −9.985 | 1.00 | 122.26 | A | C |
| ATOM | 3177 | CG1 | VAL | C | 45 | −18.738 | 21.274 | −8.692 | 1.00 | 132.55 | A | C |
| ATOM | 3178 | CG2 | VAL | C | 45 | −17.875 | 20.038 | −10.671 | 1.00 | 105.66 | A | C |
| ATOM | 3179 | C | VAL | C | 45 | −19.454 | 23.413 | −10.228 | 1.00 | 135.04 | A | C |
| ATOM | 3180 | O | VAL | C | 45 | −20.681 | 23.241 | −10.241 | 1.00 | 135.60 | A | O |
| ATOM | 3181 | N | VAL | C | 46 | −18.865 | 24.445 | −9.615 | 1.00 | 133.59 | A | N |
| ATOM | 3182 | CA | VAL | C | 46 | −19.675 | 25.547 | −9.081 | 1.00 | 141.97 | A | C |
| ATOM | 3183 | CB | VAL | C | 46 | −18.859 | 26.583 | −8.284 | 1.00 | 119.74 | A | C |
| ATOM | 3184 | CG1 | VAL | C | 46 | −17.768 | 25.877 | −7.517 | 1.00 | 122.06 | A | C |
| ATOM | 3185 | CG2 | VAL | C | 46 | −18.222 | 27.595 | −9.202 | 1.00 | 133.56 | A | C |
| ATOM | 3186 | C | VAL | C | 46 | −20.399 | 26.187 | −10.255 | 1.00 | 141.43 | A | C |
| ATOM | 3187 | O | VAL | C | 46 | −21.597 | 26.448 | −10.194 | 1.00 | 168.14 | A | O |
| ATOM | 3188 | N | VAL | C | 47 | −19.675 | 26.376 | −11.346 | 1.00 | 131.34 | A | N |
| ATOM | 3189 | CA | VAL | C | 47 | −20.238 | 26.976 | −12.543 | 1.00 | 140.77 | A | C |
| ATOM | 3190 | CB | VAL | C | 47 | −19.144 | 27.186 | −13.600 | 1.00 | 138.47 | A | C |
| ATOM | 3191 | CG1 | VAL | C | 47 | −19.734 | 27.545 | −14.942 | 1.00 | 125.29 | A | C |
| ATOM | 3192 | CG2 | VAL | C | 47 | −18.189 | 28.265 | −13.132 | 1.00 | 156.14 | A | C |
| ATOM | 3193 | C | VAL | C | 47 | −21.382 | 26.149 | −13.117 | 1.00 | 140.44 | A | C |
| ATOM | 3194 | O | VAL | C | 47 | −22.392 | 26.698 | −13.542 | 1.00 | 147.97 | A | O |
| ATOM | 3195 | N | ILE | C | 48 | −21.220 | 24.833 | −13.128 | 1.00 | 128.01 | A | N |
| ATOM | 3196 | CA | ILE | C | 48 | −22.289 | 23.947 | −13.531 | 1.00 | 139.96 | A | C |
| ATOM | 3197 | CB | ILE | C | 48 | −21.858 | 22.474 | −13.496 | 1.00 | 143.31 | A | C |
| ATOM | 3198 | CG1 | ILE | C | 48 | −21.008 | 22.149 | −14.721 | 1.00 | 161.65 | A | C |
| ATOM | 3199 | CD1 | ILE | C | 48 | −20.108 | 20.955 | −14.526 | 1.00 | 170.67 | A | C |
| ATOM | 3200 | CG2 | ILE | C | 48 | −23.076 | 21.560 | −13.467 | 1.00 | 164.63 | A | C |
| ATOM | 3201 | C | ILE | C | 48 | −23.448 | 24.122 | −12.587 | 1.00 | 140.71 | A | C |
| ATOM | 3202 | O | ILE | C | 48 | −24.578 | 24.266 | −13.031 | 1.00 | 167.05 | A | O |
| ATOM | 3203 | N | THR | C | 49 | −23.166 | 24.114 | −11.290 | 1.00 | 135.23 | A | N |
| ATOM | 3204 | CA | THR | C | 49 | −24.225 | 24.242 | −10.302 | 1.00 | 152.70 | A | C |
| ATOM | 3205 | CB | THR | C | 49 | −23.722 | 23.951 | −8.867 | 1.00 | 137.66 | A | C |
| ATOM | 3206 | OG1 | THR | C | 49 | −22.651 | 24.836 | −8.534 | 1.00 | 148.42 | A | O |
| ATOM | 3207 | CG2 | THR | C | 49 | −23.218 | 22.541 | −8.762 | 1.00 | 147.08 | A | C |
| ATOM | 3208 | C | THR | C | 49 | −24.913 | 25.612 | −10.378 | 1.00 | 156.65 | A | C |
| ATOM | 3209 | O | THR | C | 49 | −26.105 | 25.720 | −10.138 | 1.00 | 160.11 | A | O |
| ATOM | 3210 | N | TYR | C | 50 | −24.166 | 26.650 | −10.737 | 1.00 | 152.47 | A | N |
| ATOM | 3211 | CA | TYR | C | 50 | −24.713 | 28.009 | −10.773 | 1.00 | 156.47 | A | C |
| ATOM | 3212 | CB | TYR | C | 50 | −23.615 | 29.053 | −10.899 | 1.00 | 204.20 | A | C |
| ATOM | 3213 | CG | TYR | C | 50 | −23.111 | 29.491 | −9.554 | 1.00 | 241.67 | A | C |
| ATOM | 3214 | CD1 | TYR | C | 50 | −22.045 | 28.820 | −8.938 | 1.00 | 223.14 | A | C |
| ATOM | 3215 | CE1 | TYR | C | 50 | −21.580 | 29.210 | −7.693 | 1.00 | 221.29 | A | C |
| ATOM | 3216 | CZ | TYR | C | 50 | −22.196 | 30.277 | −7.044 | 1.00 | 252.60 | A | C |
| ATOM | 3217 | OH | TYR | C | 50 | −21.738 | 30.658 | −5.808 | 1.00 | 253.54 | A | O |
| ATOM | 3218 | CE2 | TYR | C | 50 | −23.270 | 30.945 | −7.623 | 1.00 | 225.07 | A | C |
| ATOM | 3219 | CD2 | TYR | C | 50 | −23.718 | 30.556 | −8.875 | 1.00 | 246.43 | A | C |
| ATOM | 3220 | C | TYR | C | 50 | −25.723 | 28.251 | −11.853 | 1.00 | 171.66 | A | C |
| ATOM | 3221 | O | TYR | C | 50 | −26.595 | 29.107 | −11.695 | 1.00 | 190.23 | A | O |
| ATOM | 3222 | N | SER | C | 51 | −25.581 | 27.526 | −12.960 | 1.00 | 164.60 | A | N |
| ATOM | 3223 | CA | SER | C | 51 | −26.595 | 27.551 | −14.009 | 1.00 | 182.59 | A | C |
| ATOM | 3224 | CB | SER | C | 51 | −25.977 | 27.368 | −15.401 | 1.00 | 177.03 | A | C |
| ATOM | 3225 | OG | SER | C | 51 | −25.068 | 26.295 | −15.417 | 1.00 | 197.67 | A | O |
| ATOM | 3226 | C | SER | C | 51 | −27.675 | 26.514 | −13.708 | 1.00 | 172.06 | A | C |
| ATOM | 3227 | O | SER | C | 51 | −28.853 | 26.737 | −13.968 | 1.00 | 167.83 | A | O |
| ATOM | 3228 | N | ALA | C | 52 | −27.276 | 25.400 | −13.112 | 1.00 | 176.31 | A | N |
| ATOM | 3229 | CA | ALA | C | 52 | −28.246 | 24.466 | −12.541 | 1.00 | 183.17 | A | C |
| ATOM | 3230 | CB | ALA | C | 52 | −27.537 | 23.308 | −11.869 | 1.00 | 196.04 | A | C |
| ATOM | 3231 | C | ALA | C | 52 | −29.163 | 25.187 | −11.547 | 1.00 | 191.46 | A | C |
| ATOM | 3232 | O | ALA | C | 52 | −30.323 | 24.792 | −11.365 | 1.00 | 226.29 | A | O |
| ATOM | 3233 | N | LEU | C | 53 | −28.626 | 26.234 | −10.914 | 1.00 | 174.77 | A | N |
| ATOM | 3234 | CA | LEU | C | 53 | −29.416 | 27.159 | −10.101 | 1.00 | 172.65 | A | C |
| ATOM | 3235 | CB | LEU | C | 53 | −28.502 | 28.176 | −9.424 | 1.00 | 202.22 | A | C |
| ATOM | 3236 | CG | LEU | C | 53 | −28.096 | 28.018 | −7.967 | 1.00 | 224.39 | A | C |
| ATOM | 3237 | CD1 | LEU | C | 53 | −26.685 | 28.572 | −7.809 | 1.00 | 243.66 | A | C |
| ATOM | 3238 | CD2 | LEU | C | 53 | −29.108 | 28.708 | −7.043 | 1.00 | 210.89 | A | C |
| ATOM | 3239 | C | LEU | C | 53 | −30.408 | 27.947 | −10.933 | 1.00 | 180.97 | A | C |
| ATOM | 3240 | O | LEU | C | 53 | −31.597 | 28.001 | −10.612 | 1.00 | 213.58 | A | O |
| ATOM | 3241 | N | HIS | C | 54 | −29.902 | 28.579 | −11.988 | 1.00 | 164.23 | A | N |
| ATOM | 3242 | CA | HIS | C | 54 | −30.662 | 29.541 | −12.736 | 1.00 | 170.04 | A | C |
| ATOM | 3243 | CB | HIS | C | 54 | −29.944 | 30.883 | −12.758 | 1.00 | 203.64 | A | C |
| ATOM | 3244 | CG | HIS | C | 54 | −29.533 | 31.366 | −11.391 | 1.00 | 221.05 | A | C |
| ATOM | 3245 | ND1 | HIS | C | 54 | −28.251 | 31.339 | −10.962 | 1.00 | 224.44 | A | N |
| ATOM | 3246 | CE1 | HIS | C | 54 | −28.194 | 31.817 | −9.704 | 1.00 | 242.92 | A | C |
| ATOM | 3247 | NE2 | HIS | C | 54 | −29.442 | 32.140 | −9.324 | 1.00 | 273.55 | A | N |
| ATOM | 3248 | CD2 | HIS | C | 54 | −30.291 | 31.864 | −10.336 | 1.00 | 233.30 | A | C |
| ATOM | 3249 | C | HIS | C | 54 | −30.824 | 29.017 | −14.104 | 1.00 | 206.80 | A | C |
| ATOM | 3250 | O | HIS | C | 54 | −29.976 | 29.286 | −14.965 | 1.00 | 212.48 | A | O |
| ATOM | 3251 | N | PRO | C | 55 | −31.903 | 28.232 | −14.323 | 1.00 | 203.37 | A | N |
| ATOM | 3252 | CA | PRO | C | 55 | −32.183 | 27.657 | −15.643 | 1.00 | 234.74 | A | C |
| ATOM | 3253 | CB | PRO | C | 55 | −33.506 | 26.908 | −15.416 | 1.00 | 240.87 | A | C |
| ATOM | 3254 | CG | PRO | C | 55 | −33.533 | 26.601 | −13.944 | 1.00 | 176.82 | A | C |
| ATOM | 3255 | CD | PRO | C | 55 | −32.872 | 27.777 | −13.302 | 1.00 | 159.94 | A | C |
| ATOM | 3256 | C | PRO | C | 55 | −32.319 | 28.732 | −16.743 | 1.00 | 249.41 | A | C |
| ATOM | 3257 | O | PRO | C | 55 | −32.936 | 28.488 | −17.780 | 1.00 | 239.96 | A | O |
| ATOM | 3258 | N | THR | C | 56 | −31.715 | 29.902 | −16.511 | 1.00 | 254.08 | A | N |
| ATOM | 3259 | CA | THR | C | 56 | −31.995 | 31.125 | −17.276 | 1.00 | 246.97 | A | C |
| ATOM | 3260 | CB | THR | C | 56 | −31.246 | 31.177 | −18.631 | 1.00 | 217.43 | A | C |
| ATOM | 3261 | OG1 | THR | C | 56 | −31.771 | 30.175 | −19.509 | 1.00 | 271.31 | A | O |
| ATOM | 3262 | CG2 | THR | C | 56 | −29.736 | 30.961 | −18.444 | 1.00 | 229.67 | A | C |
| ATOM | 3263 | C | THR | C | 56 | −33.507 | 31.293 | −17.466 | 1.00 | 247.26 | A | C |
| ATOM | 3264 | O | THR | C | 56 | −33.958 | 31.953 | −18.397 | 1.00 | 265.63 | A | O |
| ATOM | 3265 | N | ALA | C | 57 | −34.276 | 30.667 | −16.577 | 1.00 | 236.00 | A | N |
| ATOM | 3266 | CA | ALA | C | 57 | −35.721 | 30.860 | −16.503 | 1.00 | 245.21 | A | C |
| ATOM | 3267 | CB | ALA | C | 57 | −36.404 | 29.624 | −15.939 | 1.00 | 252.51 | A | C |
| ATOM | 3268 | C | ALA | C | 57 | −36.052 | 32.095 | −15.661 | 1.00 | 254.43 | A | C |
| ATOM | 3269 | O | ALA | C | 57 | −37.079 | 32.735 | −15.903 | 1.00 | 253.35 | A | O |
| ATOM | 3270 | N | PRO | C | 58 | −35.194 | 32.426 | −14.664 | 1.00 | 260.86 | A | N |
| ATOM | 3271 | CA | PRO | C | 58 | −35.335 | 33.669 | −13.902 | 1.00 | 277.13 | A | C |
| ATOM | 3272 | CB | PRO | C | 58 | −34.186 | 33.589 | −12.881 | 1.00 | 249.15 | A | C |
| ATOM | 3273 | CG | PRO | C | 58 | −33.213 | 32.612 | −13.450 | 1.00 | 199.53 | A | C |
| ATOM | 3274 | CD | PRO | C | 58 | −34.100 | 31.602 | −14.118 | 1.00 | 237.28 | A | C |
| ATOM | 3275 | C | PRO | C | 58 | −35.159 | 34.904 | −14.785 | 1.00 | 256.34 | A | C |
| ATOM | 3276 | O | PRO | C | 58 | −35.312 | 36.033 | −14.315 | 1.00 | 276.42 | A | O |
| ATOM | 3277 | N | SER | C | 74 | −25.851 | 28.834 | −23.375 | 1.00 | 167.09 | A | N |
| ATOM | 3278 | CA | SER | C | 74 | −26.079 | 27.900 | −22.262 | 1.00 | 199.20 | A | C |
| ATOM | 3279 | CB | SER | C | 74 | −26.531 | 26.521 | −22.789 | 1.00 | 174.56 | A | C |
| ATOM | 3280 | OG | SER | C | 74 | −27.108 | 26.589 | −24.088 | 1.00 | 154.02 | A | O |
| ATOM | 3281 | C | SER | C | 74 | −24.827 | 27.737 | −21.377 | 1.00 | 169.05 | A | C |
| ATOM | 3282 | O | SER | C | 74 | −23.962 | 28.619 | −21.329 | 1.00 | 162.32 | A | O |
| ATOM | 3283 | N | PHE | C | 75 | −24.741 | 26.603 | −20.683 | 1.00 | 171.47 | A | N |
| ATOM | 3284 | CA | PHE | C | 75 | −23.523 | 26.215 | −19.970 | 1.00 | 186.44 | A | C |
| ATOM | 3285 | CB | PHE | C | 75 | −23.834 | 25.269 | −18.796 | 1.00 | 185.98 | A | C |
| ATOM | 3286 | CG | PHE | C | 75 | −24.745 | 24.121 | −19.146 | 1.00 | 188.65 | A | C |
| ATOM | 3287 | CD1 | PHE | C | 75 | −24.310 | 23.048 | −19.932 | 1.00 | 183.90 | A | C |
| ATOM | 3288 | CE1 | PHE | C | 75 | −25.177 | 22.009 | −20.253 | 1.00 | 187.43 | A | C |
| ATOM | 3289 | CZ | PHE | C | 75 | −26.470 | 22.013 | −19.756 | 1.00 | 203.92 | A | C |
| ATOM | 3290 | CE2 | PHE | C | 75 | −26.900 | 23.053 | −18.950 | 1.00 | 199.53 | A | C |
| ATOM | 3291 | CD2 | PHE | C | 75 | −26.040 | 24.096 | −18.649 | 1.00 | 184.33 | A | C |
| ATOM | 3292 | C | PHE | C | 75 | −22.480 | 25.602 | −20.925 | 1.00 | 163.18 | A | C |
| ATOM | 3293 | O | PHE | C | 75 | −21.553 | 24.887 | −20.519 | 1.00 | 153.04 | A | O |
| ATOM | 3294 | N | TYR | C | 76 | −22.653 | 25.884 | −22.210 | 1.00 | 178.45 | A | N |
| ATOM | 3295 | CA | TYR | C | 76 | −21.625 | 25.643 | −23.220 | 1.00 | 180.75 | A | C |
| ATOM | 3296 | CB | TYR | C | 76 | −21.951 | 26.463 | −24.470 | 1.00 | 166.94 | A | C |
| ATOM | 3297 | CG | TYR | C | 76 | −20.832 | 26.613 | −25.477 | 1.00 | 197.29 | A | C |
| ATOM | 3298 | CD1 | TYR | C | 76 | −20.591 | 25.628 | −26.445 | 1.00 | 187.29 | A | C |
| ATOM | 3299 | CE1 | TYR | C | 76 | −19.583 | 25.783 | −27.391 | 1.00 | 219.99 | A | C |
| ATOM | 3300 | CZ | TYR | C | 76 | −18.799 | 26.932 | −27.373 | 1.00 | 249.63 | A | C |
| ATOM | 3301 | OH | TYR | C | 76 | −17.788 | 27.095 | −28.296 | 1.00 | 280.05 | A | O |
| ATOM | 3302 | CE2 | TYR | C | 76 | −19.021 | 27.920 | −26.423 | 1.00 | 258.59 | A | C |
| ATOM | 3303 | CD2 | TYR | C | 76 | −20.037 | 27.763 | −25.489 | 1.00 | 217.69 | A | C |
| ATOM | 3304 | C | TYR | C | 76 | −20.215 | 25.981 | −22.706 | 1.00 | 173.56 | A | C |
| ATOM | 3305 | O | TYR | C | 76 | −19.234 | 25.367 | −23.107 | 1.00 | 150.64 | A | O |
| ATOM | 3306 | N | GLY | C | 77 | −20.133 | 26.973 | −21.826 | 1.00 | 183.21 | A | N |
| ATOM | 3307 | CA | GLY | C | 77 | −18.860 | 27.432 | −21.282 | 1.00 | 159.32 | A | C |
| ATOM | 3308 | C | GLY | C | 77 | −18.146 | 26.380 | −20.469 | 1.00 | 162.29 | A | C |
| ATOM | 3309 | O | GLY | C | 77 | −16.987 | 26.093 | −20.742 | 1.00 | 182.09 | A | O |
| ATOM | 3310 | N | PRO | C | 78 | −18.825 | 25.815 | −19.449 | 1.00 | 159.78 | A | N |
| ATOM | 3311 | CA | PRO | C | 78 | −18.298 | 24.662 | −18.705 | 1.00 | 154.14 | A | C |
| ATOM | 3312 | CB | PRO | C | 78 | −19.483 | 24.216 | −17.847 | 1.00 | 141.48 | A | C |
| ATOM | 3313 | CG | PRO | C | 78 | −20.246 | 25.473 | −17.612 | 1.00 | 151.98 | A | C |
| ATOM | 3314 | CD | PRO | C | 78 | −19.951 | 26.443 | −18.738 | 1.00 | 157.26 | A | C |
| ATOM | 3315 | C | PRO | C | 78 | −17.868 | 23.535 | −19.616 | 1.00 | 152.23 | A | C |
| ATOM | 3316 | O | PRO | C | 78 | −16.816 | 22.930 | −19.379 | 1.00 | 166.20 | A | O |
| ATOM | 3317 | N | ALA | C | 79 | −18.672 | 23.255 | −20.639 | 1.00 | 136.73 | A | N |
| ATOM | 3318 | CA | ALA | C | 79 | −18.332 | 22.230 | −21.605 | 1.00 | 152.48 | A | C |
| ATOM | 3319 | CB | ALA | C | 79 | −19.373 | 22.166 | −22.717 | 1.00 | 158.94 | A | C |
| ATOM | 3320 | C | ALA | C | 79 | −16.952 | 22.539 | −22.165 | 1.00 | 171.50 | A | C |
| ATOM | 3321 | O | ALA | C | 79 | −16.045 | 21.721 | −22.055 | 1.00 | 176.18 | A | O |
| ATOM | 3322 | N | THR | C | 80 | −16.785 | 23.739 | −22.720 | 1.00 | 181.00 | A | N |
| ATOM | 3323 | CA | THR | C | 80 | −15.484 | 24.181 | −23.213 | 1.00 | 179.17 | A | C |
| ATOM | 3324 | CB | THR | C | 80 | −15.569 | 25.562 | −23.886 | 1.00 | 204.39 | A | C |
| ATOM | 3325 | OG1 | THR | C | 80 | −15.973 | 26.553 | −22.922 | 1.00 | 212.83 | A | O |
| ATOM | 3326 | CG2 | THR | C | 80 | −16.567 | 25.535 | −25.053 | 1.00 | 235.74 | A | C |
| ATOM | 3327 | C | THR | C | 80 | −14.447 | 24.232 | −22.087 | 1.00 | 184.28 | A | C |
| ATOM | 3328 | O | THR | C | 80 | −13.297 | 23.838 | −22.277 | 1.00 | 159.88 | A | O |
| ATOM | 3329 | N | GLY | C | 81 | −14.867 | 24.712 | −20.917 | 1.00 | 177.32 | A | N |
| ATOM | 3330 | CA | GLY | C | 81 | −13.976 | 24.850 | −19.766 | 1.00 | 165.58 | A | C |
| ATOM | 3331 | C | GLY | C | 81 | −13.267 | 23.561 | −19.398 | 1.00 | 156.18 | A | C |
| ATOM | 3332 | O | GLY | C | 81 | −12.043 | 23.425 | −19.569 | 1.00 | 156.86 | A | O |
| ATOM | 3333 | N | LEU | C | 82 | −14.042 | 22.598 | −18.907 | 1.00 | 130.10 | A | N |
| ATOM | 3334 | CA | LEU | C | 82 | −13.473 | 21.357 | −18.402 | 1.00 | 145.75 | A | C |
| ATOM | 3335 | CB | LEU | C | 82 | −14.414 | 20.689 | −17.402 | 1.00 | 138.16 | A | C |
| ATOM | 3336 | CG | LEU | C | 82 | −15.753 | 20.185 | −17.894 | 1.00 | 132.21 | A | C |
| ATOM | 3337 | CD1 | LEU | C | 82 | −15.624 | 18.705 | −18.175 | 1.00 | 142.00 | A | C |
| ATOM | 3338 | CD2 | LEU | C | 82 | −16.755 | 20.384 | −16.783 | 1.00 | 145.08 | A | C |
| ATOM | 3339 | C | LEU | C | 82 | −13.067 | 20.413 | −19.526 | 1.00 | 152.56 | A | C |
| ATOM | 3340 | O | LEU | C | 82 | −12.603 | 19.294 | −19.285 | 1.00 | 168.40 | A | O |
| ATOM | 3341 | N | LEU | C | 83 | −13.236 | 20.873 | −20.753 | 1.00 | 144.58 | A | N |
| ATOM | 3342 | CA | LEU | C | 83 | −12.674 | 20.172 | −21.890 | 1.00 | 151.84 | A | C |
| ATOM | 3343 | CB | LEU | C | 83 | −12.989 | 20.908 | −23.189 | 1.00 | 167.98 | A | C |
| ATOM | 3344 | CG | LEU | C | 83 | −12.625 | 20.080 | −24.412 | 1.00 | 172.20 | A | C |
| ATOM | 3345 | CD1 | LEU | C | 83 | −13.549 | 18.878 | −24.532 | 1.00 | 172.51 | A | C |
| ATOM | 3346 | CD2 | LEU | C | 83 | −12.677 | 20.953 | −25.645 | 1.00 | 203.85 | A | C |
| ATOM | 3347 | C | LEU | C | 83 | −11.165 | 19.977 | −21.716 | 1.00 | 151.91 | A | C |
| ATOM | 3348 | O | LEU | C | 83 | −10.686 | 18.847 | −21.677 | 1.00 | 150.25 | A | O |
| ATOM | 3349 | N | PHE | C | 84 | −10.423 | 21.074 | −21.585 | 1.00 | 151.66 | A | N |
| ATOM | 3350 | CA | PHE | C | 84 | −8.998 | 20.964 | −21.306 | 1.00 | 164.82 | A | C |
| ATOM | 3351 | CB | PHE | C | 84 | −8.154 | 21.974 | −22.099 | 1.00 | 169.03 | A | C |
| ATOM | 3352 | CG | PHE | C | 84 | −8.685 | 23.369 | −22.097 | 1.00 | 177.61 | A | C |
| ATOM | 3353 | CD1 | PHE | C | 84 | −8.337 | 24.268 | −21.088 | 1.00 | 195.55 | A | C |
| ATOM | 3354 | CE1 | PHE | C | 84 | −8.816 | 25.573 | −21.107 | 1.00 | 210.29 | A | C |
| ATOM | 3355 | CZ | PHE | C | 84 | −9.637 | 25.994 | −22.149 | 1.00 | 222.83 | A | C |
| ATOM | 3356 | CE2 | PHE | C | 84 | −9.979 | 25.108 | −23.166 | 1.00 | 214.74 | A | C |
| ATOM | 3357 | CD2 | PHE | C | 84 | −9.493 | 23.809 | −23.143 | 1.00 | 196.94 | A | C |
| ATOM | 3358 | C | PHE | C | 84 | −8.669 | 20.972 | −19.818 | 1.00 | 153.77 | A | C |
| ATOM | 3359 | O | PHE | C | 84 | −7.521 | 20.781 | −19.420 | 1.00 | 155.67 | A | O |
| ATOM | 3360 | N | GLY | C | 85 | −9.683 | 21.164 | −18.995 | 1.00 | 143.28 | A | N |
| ATOM | 3361 | CA | GLY | C | 85 | −9.573 | 20.776 | −17.601 | 1.00 | 134.91 | A | C |
| ATOM | 3362 | C | GLY | C | 85 | −9.122 | 19.328 | −17.468 | 1.00 | 139.02 | A | C |
| ATOM | 3363 | O | GLY | C | 85 | −8.146 | 19.045 | −16.788 | 1.00 | 130.30 | A | O |
| ATOM | 3364 | N | PHE | C | 86 | −9.829 | 18.422 | −18.144 | 1.00 | 135.15 | A | N |
| ATOM | 3365 | CA | PHE | C | 86 | −9.505 | 17.007 | −18.134 | 1.00 | 132.71 | A | C |
| ATOM | 3366 | CB | PHE | C | 86 | −10.533 | 16.208 | −18.922 | 1.00 | 137.73 | A | C |
| ATOM | 3367 | CG | PHE | C | 86 | −11.810 | 15.980 | −18.179 | 1.00 | 148.81 | A | C |
| ATOM | 3368 | CD1 | PHE | C | 86 | −11.846 | 16.090 | −16.800 | 1.00 | 180.20 | A | C |
| ATOM | 3369 | CE1 | PHE | C | 86 | −13.022 | 15.870 | −16.096 | 1.00 | 176.48 | A | C |
| ATOM | 3370 | CZ | PHE | C | 86 | −14.181 | 15.530 | −16.777 | 1.00 | 157.57 | A | C |
| ATOM | 3371 | CE2 | PHE | C | 86 | −14.151 | 15.405 | −18.154 | 1.00 | 166.14 | A | C |
| ATOM | 3372 | CD2 | PHE | C | 86 | −12.971 | 15.628 | −18.849 | 1.00 | 162.11 | A | C |
| ATOM | 3373 | C | PHE | C | 86 | −8.159 | 16.744 | −18.726 | 1.00 | 117.33 | A | C |
| ATOM | 3374 | O | PHE | C | 86 | −7.393 | 15.960 | −18.190 | 1.00 | 126.12 | A | O |
| ATOM | 3375 | N | THR | C | 87 | −7.877 | 17.413 | −19.836 | 1.00 | 118.25 | A | N |
| ATOM | 3376 | CA | THR | C | 87 | −6.642 | 17.196 | −20.569 | 1.00 | 117.97 | A | C |
| ATOM | 3377 | CB | THR | C | 87 | −6.443 | 18.253 | −21.639 | 1.00 | 129.15 | A | C |
| ATOM | 3378 | OG1 | THR | C | 87 | −7.478 | 18.121 | −22.615 | 1.00 | 154.65 | A | O |
| ATOM | 3379 | CG2 | THR | C | 87 | −5.094 | 18.067 | −22.324 | 1.00 | 168.73 | A | C |
| ATOM | 3380 | C | THR | C | 87 | −5.449 | 17.233 | −19.639 | 1.00 | 148.61 | A | C |
| ATOM | 3381 | O | THR | C | 87 | −4.517 | 16.445 | −19.814 | 1.00 | 150.61 | A | O |
| ATOM | 3382 | N | TYR | C | 88 | −5.479 | 18.146 | −18.665 | 1.00 | 152.24 | A | N |
| ATOM | 3383 | CA | TYR | C | 88 | −4.396 | 18.273 | −17.695 | 1.00 | 129.43 | A | C |
| ATOM | 3384 | CB | TYR | C | 88 | −4.468 | 19.607 | −16.956 | 1.00 | 136.27 | A | C |
| ATOM | 3385 | CG | TYR | C | 88 | −4.263 | 20.775 | −17.872 | 1.00 | 170.56 | A | C |
| ATOM | 3386 | CD1 | TYR | C | 88 | −3.100 | 20.875 | −18.648 | 1.00 | 172.05 | A | C |
| ATOM | 3387 | CE1 | TYR | C | 88 | −2.900 | 21.928 | −19.512 | 1.00 | 168.48 | A | C |
| ATOM | 3388 | CZ | TYR | C | 88 | −3.880 | 22.917 | −19.616 | 1.00 | 215.40 | A | C |
| ATOM | 3389 | OH | TYR | C | 88 | −3.693 | 23.995 | −20.475 | 1.00 | 191.35 | A | O |
| ATOM | 3390 | CE2 | TYR | C | 88 | −5.045 | 22.829 | −18.851 | 1.00 | 183.47 | A | C |
| ATOM | 3391 | CD2 | TYR | C | 88 | −5.227 | 21.765 | −17.987 | 1.00 | 150.36 | A | C |
| ATOM | 3392 | C | TYR | C | 88 | −4.410 | 17.123 | −16.734 | 1.00 | 113.69 | A | C |
| ATOM | 3393 | O | TYR | C | 88 | −3.399 | 16.459 | −16.532 | 1.00 | 109.29 | A | O |
| ATOM | 3394 | N | LEU | C | 89 | −5.578 | 16.887 | −16.155 | 1.00 | 134.27 | A | N |
| ATOM | 3395 | CA | LEU | C | 89 | −5.781 | 15.780 | −15.254 | 1.00 | 123.12 | A | C |
| ATOM | 3396 | CB | LEU | C | 89 | −7.201 | 15.818 | −14.712 | 1.00 | 114.46 | A | C |
| ATOM | 3397 | CG | LEU | C | 89 | −7.548 | 14.736 | −13.717 | 1.00 | 114.28 | A | C |
| ATOM | 3398 | CD1 | LEU | C | 89 | −6.480 | 14.636 | −12.655 | 1.00 | 145.16 | A | C |
| ATOM | 3399 | CD2 | LEU | C | 89 | −8.850 | 15.060 | −13.031 | 1.00 | 158.02 | A | C |
| ATOM | 3400 | C | LEU | C | 89 | −5.473 | 14.458 | −15.958 | 1.00 | 125.23 | A | C |
| ATOM | 3401 | O | LEU | C | 89 | −4.889 | 13.563 | −15.363 | 1.00 | 126.58 | A | O |
| ATOM | 3402 | N | TYR | C | 90 | −5.837 | 14.352 | −17.229 | 1.00 | 120.68 | A | N |
| ATOM | 3403 | CA | TYR | C | 90 | −5.469 | 13.199 | −18.046 | 1.00 | 129.69 | A | C |
| ATOM | 3404 | CB | TYR | C | 90 | −6.004 | 13.332 | −19.476 | 1.00 | 135.49 | A | C |
| ATOM | 3405 | CG | TYR | C | 90 | −6.472 | 12.017 | −20.094 | 1.00 | 141.49 | A | C |
| ATOM | 3406 | CD1 | TYR | C | 90 | −5.603 | 10.943 | −20.267 | 1.00 | 155.56 | A | C |
| ATOM | 3407 | CE1 | TYR | C | 90 | −6.032 | 9.740 | −20.830 | 1.00 | 165.42 | A | C |
| ATOM | 3408 | CZ | TYR | C | 90 | −7.344 | 9.609 | −21.249 | 1.00 | 171.70 | A | C |
| ATOM | 3409 | OH | TYR | C | 90 | −7.788 | 8.429 | −21.813 | 1.00 | 157.52 | A | O |
| ATOM | 3410 | CE2 | TYR | C | 90 | −8.213 | 10.670 | −21.111 | 1.00 | 173.13 | A | C |
| ATOM | 3411 | CD2 | TYR | C | 90 | −7.780 | 11.864 | −20.543 | 1.00 | 156.33 | A | C |
| ATOM | 3412 | C | TYR | C | 90 | −3.956 | 13.038 | −18.076 | 1.00 | 125.90 | A | C |
| ATOM | 3413 | O | TYR | C | 90 | −3.435 | 11.943 | −17.882 | 1.00 | 126.59 | A | O |
| ATOM | 3414 | N | ALA | C | 91 | −3.248 | 14.133 | −18.305 | 1.00 | 115.29 | A | N |
| ATOM | 3415 | CA | ALA | C | 91 | −1.799 | 14.091 | −18.329 | 1.00 | 115.12 | A | C |
| ATOM | 3416 | CB | ALA | C | 91 | −1.229 | 15.445 | −18.672 | 1.00 | 126.17 | A | C |
| ATOM | 3417 | C | ALA | C | 91 | −1.283 | 13.662 | −16.985 | 1.00 | 108.12 | A | C |
| ATOM | 3418 | O | ALA | C | 91 | −0.511 | 12.729 | −16.908 | 1.00 | 119.31 | A | O |
| ATOM | 3419 | N | ALA | C | 92 | −1.723 | 14.347 | −15.934 | 1.00 | 109.19 | A | N |
| ATOM | 3420 | CA | ALA | C | 92 | −1.237 | 14.108 | −14.596 | 1.00 | 106.84 | A | C |
| ATOM | 3421 | CB | ALA | C | 92 | −2.049 | 14.911 | −13.603 | 1.00 | 107.84 | A | C |
| ATOM | 3422 | C | ALA | C | 92 | −1.307 | 12.628 | −14.286 | 1.00 | 115.05 | A | C |
| ATOM | 3423 | O | ALA | C | 92 | −0.314 | 12.026 | −13.890 | 1.00 | 118.56 | A | O |
| ATOM | 3424 | N | ILE | C | 93 | −2.472 | 12.033 | −14.516 | 1.00 | 120.00 | A | N |
| ATOM | 3425 | CA | ILE | C | 93 | −2.704 | 10.630 | −14.176 | 1.00 | 121.19 | A | C |
| ATOM | 3426 | CB | ILE | C | 93 | −4.212 | 10.298 | −14.225 | 1.00 | 114.79 | A | C |
| ATOM | 3427 | CG1 | ILE | C | 93 | −4.875 | 10.827 | −12.959 | 1.00 | 105.03 | A | C |
| ATOM | 3428 | CD1 | ILE | C | 93 | −6.371 | 10.674 | −12.925 | 1.00 | 102.18 | A | C |
| ATOM | 3429 | CG2 | ILE | C | 93 | −4.453 | 8.807 | −14.316 | 1.00 | 108.97 | A | C |
| ATOM | 3430 | C | ILE | C | 93 | −1.869 | 9.701 | −15.054 | 1.00 | 102.57 | A | C |
| ATOM | 3431 | O | ILE | C | 93 | −1.206 | 8.813 | −14.560 | 1.00 | 100.18 | A | O |
| ATOM | 3432 | N | ASN | C | 94 | −1.898 | 9.923 | −16.362 | 1.00 | 110.17 | A | N |
| ATOM | 3433 | CA | ASN | C | 94 | −1.106 | 9.111 | −17.293 | 1.00 | 119.25 | A | C |
| ATOM | 3434 | CB | ASN | C | 94 | −1.314 | 9.549 | −18.752 | 1.00 | 131.45 | A | C |
| ATOM | 3435 | CG | ASN | C | 94 | −2.342 | 8.696 | −19.494 | 1.00 | 130.94 | A | C |
| ATOM | 3436 | OD1 | ASN | C | 94 | −2.904 | 7.758 | −18.950 | 1.00 | 143.65 | A | O |
| ATOM | 3437 | ND2 | ASN | C | 94 | −2.596 | 9.026 | −20.736 | 1.00 | 123.13 | A | N |
| ATOM | 3438 | C | ASN | C | 94 | 0.374 | 9.091 | −16.929 | 1.00 | 125.45 | A | C |
| ATOM | 3439 | O | ASN | C | 94 | 1.062 | 8.088 | −17.115 | 1.00 | 123.59 | A | O |
| ATOM | 3440 | N | HIS | C | 95 | 0.846 | 10.202 | −16.379 | 1.00 | 127.74 | A | N |
| ATOM | 3441 | CA | HIS | C | 95 | 2.236 | 10.334 | −15.973 | 1.00 | 114.53 | A | C |
| ATOM | 3442 | CB | HIS | C | 95 | 2.614 | 11.796 | −15.783 | 1.00 | 117.48 | A | C |
| ATOM | 3443 | CG | HIS | C | 95 | 3.221 | 12.423 | −17.010 | 1.00 | 126.92 | A | C |
| ATOM | 3444 | ND1 | HIS | C | 95 | 2.917 | 13.668 | −17.412 | 1.00 | 144.03 | A | N |
| ATOM | 3445 | CE1 | HIS | C | 95 | 3.610 | 13.960 | −18.533 | 1.00 | 190.88 | A | C |
| ATOM | 3446 | NE2 | HIS | C | 95 | 4.347 | 12.885 | −18.854 | 1.00 | 173.48 | A | N |
| ATOM | 3447 | CD2 | HIS | C | 95 | 4.127 | 11.917 | −17.944 | 1.00 | 151.61 | A | C |
| ATOM | 3448 | C | HIS | C | 95 | 2.537 | 9.601 | −14.733 | 1.00 | 98.01 | A | C |
| ATOM | 3449 | O | HIS | C | 95 | 3.456 | 8.786 | −14.695 | 1.00 | 109.87 | A | O |
| ATOM | 3450 | N | THR | C | 96 | 1.777 | 9.895 | −13.690 | 1.00 | 82.84 | A | N |
| ATOM | 3451 | CA | THR | C | 96 | 2.062 | 9.350 | −12.378 | 1.00 | 87.28 | A | C |
| ATOM | 3452 | CB | THR | C | 96 | 1.165 | 9.944 | −11.284 | 1.00 | 109.33 | A | C |
| ATOM | 3453 | OG1 | THR | C | 96 | −0.165 | 10.037 | −11.787 | 1.00 | 120.19 | A | O |
| ATOM | 3454 | CG2 | THR | C | 96 | 1.626 | 11.348 | −10.883 | 1.00 | 99.88 | A | C |
| ATOM | 3455 | C | THR | C | 96 | 1.937 | 7.849 | −12.406 | 1.00 | 103.61 | A | C |
| ATOM | 3456 | O | THR | C | 96 | 2.735 | 7.153 | −11.799 | 1.00 | 131.95 | A | O |
| ATOM | 3457 | N | PHE | C | 97 | 0.978 | 7.342 | −13.165 | 1.00 | 96.54 | A | N |
| ATOM | 3458 | CA | PHE | C | 97 | 0.739 | 5.915 | −13.209 | 1.00 | 97.04 | A | C |
| ATOM | 3459 | CB | PHE | C | 97 | −0.750 | 5.635 | −13.215 | 1.00 | 93.50 | A | C |
| ATOM | 3460 | CG | PHE | C | 97 | −1.389 | 5.933 | −11.906 | 1.00 | 94.76 | A | C |
| ATOM | 3461 | CD1 | PHE | C | 97 | −1.723 | 7.230 | −11.591 | 1.00 | 102.87 | A | C |
| ATOM | 3462 | CE1 | PHE | C | 97 | −2.293 | 7.545 | −10.371 | 1.00 | 109.90 | A | C |
| ATOM | 3463 | CZ | PHE | C | 97 | −2.524 | 6.553 | −9.450 | 1.00 | 119.11 | A | C |
| ATOM | 3464 | CE2 | PHE | C | 97 | −2.165 | 5.254 | −9.751 | 1.00 | 109.18 | A | C |
| ATOM | 3465 | CD2 | PHE | C | 97 | −1.590 | 4.943 | −10.957 | 1.00 | 106.94 | A | C |
| ATOM | 3466 | C | PHE | C | 97 | 1.462 | 5.185 | −14.309 | 1.00 | 112.04 | A | C |
| ATOM | 3467 | O | PHE | C | 97 | 1.512 | 3.954 | −14.306 | 1.00 | 112.14 | A | O |
| ATOM | 3468 | N | GLY | C | 98 | 2.037 | 5.955 | −15.230 | 1.00 | 112.41 | A | N |
| ATOM | 3469 | CA | GLY | C | 98 | 2.912 | 5.414 | −16.269 | 1.00 | 110.81 | A | C |
| ATOM | 3470 | C | GLY | C | 98 | 2.126 | 4.591 | −17.257 | 1.00 | 106.61 | A | C |
| ATOM | 3471 | O | GLY | C | 98 | 2.308 | 3.372 | −17.376 | 1.00 | 127.48 | A | O |
| ATOM | 3472 | N | LEU | C | 99 | 1.265 | 5.287 | −17.993 | 1.00 | 141.99 | A | N |
| ATOM | 3473 | CA | LEU | C | 99 | 0.253 | 4.651 | −18.820 | 1.00 | 122.45 | A | C |
| ATOM | 3474 | CB | LEU | C | 99 | −1.142 | 5.118 | −18.362 | 1.00 | 134.69 | A | C |
| ATOM | 3475 | CG | LEU | C | 99 | −1.564 | 4.872 | −16.897 | 1.00 | 119.20 | A | C |
| ATOM | 3476 | CD1 | LEU | C | 99 | −2.584 | 5.868 | −16.460 | 1.00 | 114.55 | A | C |
| ATOM | 3477 | CD2 | LEU | C | 99 | −2.165 | 3.512 | −16.678 | 1.00 | 125.36 | A | C |
| ATOM | 3478 | C | LEU | C | 99 | 0.476 | 4.929 | −20.300 | 1.00 | 143.57 | A | C |
| ATOM | 3479 | O | LEU | C | 99 | 1.403 | 5.658 | −20.668 | 1.00 | 163.67 | A | O |
| ATOM | 3480 | N | ASP | C | 100 | −0.380 | 4.337 | −21.139 | 1.00 | 145.95 | A | N |
| ATOM | 3481 | CA | ASP | C | 100 | −0.346 | 4.497 | −22.595 | 1.00 | 145.95 | A | C |
| ATOM | 3482 | CB | ASP | C | 100 | −1.135 | 3.369 | −23.242 | 1.00 | 129.15 | A | C |
| ATOM | 3483 | CG | ASP | C | 100 | −0.378 | 2.694 | −24.356 | 1.00 | 153.74 | A | C |
| ATOM | 3484 | OD1 | ASP | C | 100 | 0.227 | 3.413 | −25.189 | 1.00 | 192.61 | A | O |
| ATOM | 3485 | OD2 | ASP | C | 100 | −0.378 | 1.439 | −24.396 | 1.00 | 164.44 | A | O |
| ATOM | 3486 | C | ASP | C | 100 | −0.920 | 5.834 | −23.064 | 1.00 | 161.28 | A | C |
| ATOM | 3487 | O | ASP | C | 100 | −2.014 | 6.244 | −22.670 | 1.00 | 176.37 | A | O |
| ATOM | 3488 | N | TRP | C | 101 | −0.182 | 6.501 | −23.935 | 1.00 | 162.40 | A | N |
| ATOM | 3489 | CA | TRP | C | 101 | −0.575 | 7.827 | −24.400 | 1.00 | 190.06 | A | C |
| ATOM | 3490 | CB | TRP | C | 101 | 0.664 | 8.667 | −24.686 | 1.00 | 188.89 | A | C |
| ATOM | 3491 | CG | TRP | C | 101 | 1.390 | 9.051 | −23.432 | 1.00 | 171.28 | A | C |
| ATOM | 3492 | CD1 | TRP | C | 101 | 2.545 | 8.483 | −22.919 | 1.00 | 193.34 | A | C |
| ATOM | 3493 | NE1 | TRP | C | 101 | 2.900 | 9.077 | −21.733 | 1.00 | 212.93 | A | N |
| ATOM | 3494 | CE2 | TRP | C | 101 | 2.010 | 10.038 | −21.395 | 1.00 | 150.36 | A | C |
| ATOM | 3495 | CD2 | TRP | C | 101 | 0.996 | 10.070 | −22.455 | 1.00 | 147.61 | A | C |
| ATOM | 3496 | CE3 | TRP | C | 101 | −0.036 | 10.997 | −22.375 | 1.00 | 165.30 | A | C |
| ATOM | 3497 | CZ3 | TRP | C | 101 | −0.076 | 11.862 | −21.270 | 1.00 | 139.65 | A | C |
| ATOM | 3498 | CH2 | TRP | C | 101 | 0.909 | 11.820 | −20.266 | 1.00 | 139.42 | A | C |
| ATOM | 3499 | CZ2 | TRP | C | 101 | 1.967 | 10.905 | −20.306 | 1.00 | 161.12 | A | C |
| ATOM | 3500 | C | TRP | C | 101 | −1.488 | 7.805 | −25.593 | 1.00 | 185.88 | A | C |
| ATOM | 3501 | O | TRP | C | 101 | −1.763 | 8.847 | −26.185 | 1.00 | 177.22 | A | O |
| ATOM | 3502 | N | ARG | C | 102 | −1.970 | 6.621 | −25.956 | 1.00 | 185.77 | A | N |
| ATOM | 3503 | CA | ARG | C | 102 | −2.871 | 6.466 | −27.099 | 1.00 | 172.57 | A | C |
| ATOM | 3504 | CB | ARG | C | 102 | −2.989 | 4.986 | −27.494 | 1.00 | 168.86 | A | C |
| ATOM | 3505 | CG | ARG | C | 102 | −1.884 | 4.504 | −28.422 | 1.00 | 175.09 | A | C |
| ATOM | 3506 | CD | ARG | C | 102 | −1.289 | 3.172 | −27.990 | 1.00 | 185.44 | A | C |
| ATOM | 3507 | NE | ARG | C | 102 | −2.090 | 2.029 | −28.412 | 1.00 | 186.59 | A | N |
| ATOM | 3508 | CZ | ARG | C | 102 | −2.015 | 0.806 | −27.885 | 1.00 | 187.52 | A | C |
| ATOM | 3509 | NH1 | ARG | C | 102 | −1.168 | 0.540 | −26.900 | 1.00 | 167.49 | A | N |
| ATOM | 3510 | NH2 | ARG | C | 102 | −2.791 | −0.166 | −28.353 | 1.00 | 274.82 | A | N |
| ATOM | 3511 | C | ARG | C | 102 | −4.244 | 7.093 | −26.816 | 1.00 | 151.94 | A | C |
| ATOM | 3512 | O | ARG | C | 102 | −4.626 | 8.064 | −27.462 | 1.00 | 157.49 | A | O |
| ATOM | 3513 | N | PRO | C | 103 | −4.977 | 6.553 | −25.825 | 1.00 | 141.78 | A | N |
| ATOM | 3514 | CA | PRO | C | 103 | −6.283 | 7.085 | −25.461 | 1.00 | 172.16 | A | C |
| ATOM | 3515 | CB | PRO | C | 103 | −6.644 | 6.273 | −24.220 | 1.00 | 178.41 | A | C |
| ATOM | 3516 | CG | PRO | C | 103 | −5.967 | 4.966 | −24.445 | 1.00 | 172.80 | A | C |
| ATOM | 3517 | CD | PRO | C | 103 | −4.640 | 5.370 | −25.017 | 1.00 | 154.89 | A | C |
| ATOM | 3518 | C | PRO | C | 103 | −6.248 | 8.571 | −25.127 | 1.00 | 165.79 | A | C |
| ATOM | 3519 | O | PRO | C | 103 | −7.232 | 9.280 | −25.355 | 1.00 | 163.17 | A | O |
| ATOM | 3520 | N | TYR | C | 104 | −5.119 | 9.030 | −24.592 | 1.00 | 168.89 | A | N |
| ATOM | 3521 | CA | TYR | C | 104 | −4.906 | 10.446 | −24.423 | 1.00 | 157.25 | A | C |
| ATOM | 3522 | CB | TYR | C | 104 | −3.624 | 10.709 | −23.650 | 1.00 | 151.27 | A | C |
| ATOM | 3523 | CG | TYR | C | 104 | −3.305 | 12.186 | −23.543 | 1.00 | 147.71 | A | C |
| ATOM | 3524 | CD1 | TYR | C | 104 | −3.920 | 12.983 | −22.569 | 1.00 | 135.89 | A | C |
| ATOM | 3525 | CE1 | TYR | C | 104 | −3.641 | 14.335 | −22.469 | 1.00 | 140.90 | A | C |
| ATOM | 3526 | CZ | TYR | C | 104 | −2.728 | 14.917 | −23.360 | 1.00 | 178.14 | A | C |
| ATOM | 3527 | OH | TYR | C | 104 | −2.484 | 16.262 | −23.216 | 1.00 | 163.29 | A | O |
| ATOM | 3528 | CE2 | TYR | C | 104 | −2.098 | 14.145 | −24.328 | 1.00 | 228.63 | A | C |
| ATOM | 3529 | CD2 | TYR | C | 104 | −2.406 | 12.793 | −24.429 | 1.00 | 157.38 | A | C |
| ATOM | 3530 | C | TYR | C | 104 | −4.824 | 11.165 | −25.765 | 1.00 | 145.95 | A | C |
| ATOM | 3531 | O | TYR | C | 104 | −5.436 | 12.206 | −25.948 | 1.00 | 168.06 | A | O |
| ATOM | 3532 | N | SER | C | 105 | −4.045 | 10.622 | −26.686 | 1.00 | 140.92 | A | N |
| ATOM | 3533 | CA | SER | C | 105 | −3.782 | 11.303 | −27.943 | 1.00 | 168.14 | A | C |
| ATOM | 3534 | CB | SER | C | 105 | −2.613 | 10.656 | −28.685 | 1.00 | 172.24 | A | C |
| ATOM | 3535 | OG | SER | C | 105 | −2.857 | 9.283 | −28.932 | 1.00 | 183.38 | A | O |
| ATOM | 3536 | C | SER | C | 105 | −5.018 | 11.346 | −28.831 | 1.00 | 184.78 | A | C |
| ATOM | 3537 | O | SER | C | 105 | −5.256 | 12.345 | −29.523 | 1.00 | 171.54 | A | O |
| ATOM | 3538 | N | TRP | C | 106 | −5.797 | 10.263 | −28.815 | 1.00 | 180.69 | A | N |
| ATOM | 3539 | CA | TRP | C | 106 | −7.085 | 10.259 | −29.497 | 1.00 | 177.93 | A | C |
| ATOM | 3540 | CB | TRP | C | 106 | −7.794 | 8.923 | −29.326 | 1.00 | 146.45 | A | C |
| ATOM | 3541 | CG | TRP | C | 106 | −7.570 | 7.946 | −30.462 | 1.00 | 174.87 | A | C |
| ATOM | 3542 | CD1 | TRP | C | 106 | −7.212 | 6.607 | −30.362 | 1.00 | 189.78 | A | C |
| ATOM | 3543 | NE1 | TRP | C | 106 | −7.120 | 6.032 | −31.611 | 1.00 | 213.82 | A | N |
| ATOM | 3544 | CE2 | TRP | C | 106 | −7.387 | 6.922 | −32.582 | 1.00 | 216.17 | A | C |
| ATOM | 3545 | CD2 | TRP | C | 106 | −7.696 | 8.190 | −31.922 | 1.00 | 194.52 | A | C |
| ATOM | 3546 | CE3 | TRP | C | 106 | −8.027 | 9.286 | −32.710 | 1.00 | 177.01 | A | C |
| ATOM | 3547 | CZ3 | TRP | C | 106 | −8.044 | 9.130 | −34.108 | 1.00 | 225.27 | A | C |
| ATOM | 3548 | CH2 | TRP | C | 106 | −7.739 | 7.914 | −34.720 | 1.00 | 229.06 | A | C |
| ATOM | 3549 | CZ2 | TRP | C | 106 | −7.409 | 6.785 | −33.967 | 1.00 | 216.16 | A | C |
| ATOM | 3550 | C | TRP | C | 106 | −7.927 | 11.375 | −28.949 | 1.00 | 157.04 | A | C |
| ATOM | 3551 | O | TRP | C | 106 | −8.447 | 12.198 | −29.696 | 1.00 | 174.80 | A | O |
| ATOM | 3552 | N | TYR | C | 107 | −8.030 | 11.436 | −27.628 | 1.00 | 150.26 | A | N |
| ATOM | 3553 | CA | TYR | C | 107 | −8.775 | 12.490 | −26.948 | 1.00 | 163.02 | A | C |
| ATOM | 3554 | CB | TYR | C | 107 | −8.598 | 12.360 | −25.427 | 1.00 | 161.13 | A | C |
| ATOM | 3555 | CG | TYR | C | 107 | −9.236 | 13.465 | −24.602 | 1.00 | 154.73 | A | C |
| ATOM | 3556 | CD1 | TYR | C | 107 | −10.590 | 13.437 | −24.288 | 1.00 | 133.70 | A | C |
| ATOM | 3557 | CE1 | TYR | C | 107 | −11.172 | 14.441 | −23.521 | 1.00 | 145.17 | A | C |
| ATOM | 3558 | CZ | TYR | C | 107 | −10.393 | 15.494 | −23.054 | 1.00 | 160.62 | A | C |
| ATOM | 3559 | OH | TYR | C | 107 | −10.951 | 16.502 | −22.299 | 1.00 | 156.71 | A | O |
| ATOM | 3560 | CE2 | TYR | C | 107 | −9.044 | 15.541 | −23.355 | 1.00 | 159.30 | A | C |
| ATOM | 3561 | CD2 | TYR | C | 107 | −8.474 | 14.531 | −24.122 | 1.00 | 169.66 | A | C |
| ATOM | 3562 | C | TYR | C | 107 | −8.335 | 13.866 | −27.425 | 1.00 | 165.07 | A | C |
| ATOM | 3563 | O | TYR | C | 107 | −9.165 | 14.727 | −27.705 | 1.00 | 188.23 | A | O |
| ATOM | 3564 | N | SER | C | 108 | −7.026 | 14.059 | −27.518 | 1.00 | 161.44 | A | N |
| ATOM | 3565 | CA | SER | C | 108 | −6.467 | 15.332 | −27.951 | 1.00 | 192.01 | A | C |
| ATOM | 3566 | CB | SER | C | 108 | −4.935 | 15.285 | −27.876 | 1.00 | 174.35 | A | C |
| ATOM | 3567 | OG | SER | C | 108 | −4.454 | 15.351 | −26.554 | 1.00 | 226.96 | A | O |
| ATOM | 3568 | C | SER | C | 108 | −6.967 | 15.759 | −29.362 | 1.00 | 204.49 | A | C |
| ATOM | 3569 | O | SER | C | 108 | −7.239 | 16.948 | −29.611 | 1.00 | 188.64 | A | O |
| ATOM | 3570 | N | LEU | C | 109 | −7.096 | 14.783 | −30.266 | 1.00 | 210.65 | A | N |
| ATOM | 3571 | CA | LEU | C | 109 | −7.721 | 15.000 | −31.585 | 1.00 | 180.85 | A | C |
| ATOM | 3572 | CB | LEU | C | 109 | −7.662 | 13.740 | −32.456 | 1.00 | 192.57 | A | C |
| ATOM | 3573 | CG | LEU | C | 109 | −8.657 | 13.669 | −33.624 | 1.00 | 196.63 | A | C |
| ATOM | 3574 | CD1 | LEU | C | 109 | −8.022 | 14.255 | −34.867 | 1.00 | 193.36 | A | C |
| ATOM | 3575 | CD2 | LEU | C | 109 | −9.154 | 12.255 | −33.886 | 1.00 | 189.58 | A | C |
| ATOM | 3576 | C | LEU | C | 109 | −9.170 | 15.432 | −31.442 | 1.00 | 197.24 | A | C |
| ATOM | 3577 | O | LEU | C | 109 | −9.567 | 16.467 | −31.980 | 1.00 | 185.27 | A | O |
| ATOM | 3578 | N | PHE | C | 110 | −9.950 | 14.618 | −30.727 | 1.00 | 199.09 | A | N |
| ATOM | 3579 | CA | PHE | C | 110 | −11.345 | 14.933 | −30.457 | 1.00 | 190.06 | A | C |
| ATOM | 3580 | CB | PHE | C | 110 | −11.922 | 13.991 | −29.390 | 1.00 | 175.19 | A | C |
| ATOM | 3581 | CG | PHE | C | 110 | −13.263 | 14.429 | −28.869 | 1.00 | 185.24 | A | C |
| ATOM | 3582 | CD1 | PHE | C | 110 | −13.365 | 15.511 | −27.987 | 1.00 | 173.16 | A | C |
| ATOM | 3583 | CE1 | PHE | C | 110 | −14.601 | 15.947 | −27.532 | 1.00 | 161.45 | A | C |
| ATOM | 3584 | CZ | PHE | C | 110 | −15.752 | 15.280 | −27.928 | 1.00 | 224.99 | A | C |
| ATOM | 3585 | CE2 | PHE | C | 110 | −15.665 | 14.194 | −28.797 | 1.00 | 205.00 | A | C |
| ATOM | 3586 | CD2 | PHE | C | 110 | −14.426 | 13.776 | −29.271 | 1.00 | 190.34 | A | C |
| ATOM | 3587 | C | PHE | C | 110 | −11.491 | 16.393 | −30.018 | 1.00 | 191.01 | A | C |
| ATOM | 3588 | O | PHE | C | 110 | −12.379 | 17.110 | −30.496 | 1.00 | 169.47 | A | O |
| ATOM | 3589 | N | VAL | C | 111 | −10.613 | 16.825 | −29.111 | 1.00 | 198.16 | A | N |
| ATOM | 3590 | CA | VAL | C | 111 | −10.628 | 18.196 | −28.610 | 1.00 | 172.51 | A | C |
| ATOM | 3591 | CB | VAL | C | 111 | −9.562 | 18.432 | −27.517 | 1.00 | 172.56 | A | C |
| ATOM | 3592 | CG1 | VAL | C | 111 | −9.401 | 19.927 | −27.226 | 1.00 | 166.54 | A | C |
| ATOM | 3593 | CG2 | VAL | C | 111 | −9.956 | 17.682 | −26.257 | 1.00 | 142.14 | A | C |
| ATOM | 3594 | C | VAL | C | 111 | −10.474 | 19.190 | −29.737 | 1.00 | 155.22 | A | C |
| ATOM | 3595 | O | VAL | C | 111 | −11.307 | 20.086 | −29.883 | 1.00 | 159.36 | A | O |
| ATOM | 3596 | N | ALA | C | 112 | −9.418 | 19.021 | −30.526 | 1.00 | 159.94 | A | N |
| ATOM | 3597 | CA | ALA | C | 112 | −9.199 | 19.855 | −31.715 | 1.00 | 209.19 | A | C |
| ATOM | 3598 | CB | ALA | C | 112 | −7.968 | 19.385 | −32.483 | 1.00 | 232.33 | A | C |
| ATOM | 3599 | C | ALA | C | 112 | −10.430 | 19.906 | −32.636 | 1.00 | 208.11 | A | C |
| ATOM | 3600 | O | ALA | C | 112 | −10.837 | 20.985 | −33.078 | 1.00 | 202.40 | A | O |
| ATOM | 3601 | N | ILE | C | 113 | −11.025 | 18.739 | −32.894 | 1.00 | 209.52 | A | N |
| ATOM | 3602 | CA | ILE | C | 113 | −12.246 | 18.649 | −33.707 | 1.00 | 199.98 | A | C |
| ATOM | 3603 | CB | ILE | C | 113 | −12.768 | 17.194 | −33.808 | 1.00 | 212.70 | A | C |
| ATOM | 3604 | CG1 | ILE | C | 113 | −11.706 | 16.275 | −34.439 | 1.00 | 204.64 | A | C |
| ATOM | 3605 | CD1 | ILE | C | 113 | −12.106 | 14.810 | −34.534 | 1.00 | 230.97 | A | C |
| ATOM | 3606 | CG2 | ILE | C | 113 | −14.059 | 17.142 | −34.620 | 1.00 | 198.85 | A | C |
| ATOM | 3607 | C | ILE | C | 113 | −13.351 | 19.578 | −33.182 | 1.00 | 198.05 | A | C |
| ATOM | 3608 | O | ILE | C | 113 | −14.170 | 20.069 | −33.947 | 1.00 | 235.67 | A | O |
| ATOM | 3609 | N | ASN | C | 114 | −13.364 | 19.832 | −31.882 | 1.00 | 180.41 | A | N |
| ATOM | 3610 | CA | ASN | C | 114 | −14.384 | 20.693 | −31.300 | 1.00 | 175.58 | A | C |
| ATOM | 3611 | CB | ASN | C | 114 | −14.935 | 20.089 | −30.006 | 1.00 | 197.22 | A | C |
| ATOM | 3612 | CG | ASN | C | 114 | −15.603 | 18.753 | −30.232 | 1.00 | 193.66 | A | C |
| ATOM | 3613 | OD1 | ASN | C | 114 | −16.620 | 18.453 | −29.601 | 1.00 | 204.93 | A | O |
| ATOM | 3614 | ND2 | ASN | C | 114 | −15.041 | 17.941 | −31.140 | 1.00 | 207.32 | A | N |
| ATOM | 3615 | C | ASN | C | 114 | −13.895 | 22.110 | −31.054 | 1.00 | 203.59 | A | C |
| ATOM | 3616 | O | ASN | C | 114 | −14.656 | 22.955 | −30.583 | 1.00 | 226.95 | A | O |
| ATOM | 3617 | N | THR | C | 115 | −12.631 | 22.366 | −31.379 | 1.00 | 214.41 | A | N |
| ATOM | 3618 | CA | THR | C | 115 | −12.086 | 23.724 | −31.329 | 1.00 | 187.37 | A | C |
| ATOM | 3619 | CB | THR | C | 115 | −10.562 | 23.738 | −31.067 | 1.00 | 182.97 | A | C |
| ATOM | 3620 | OG1 | THR | C | 115 | −9.858 | 23.203 | −32.185 | 1.00 | 228.48 | A | O |
| ATOM | 3621 | CG2 | THR | C | 115 | −10.223 | 22.944 | −29.857 | 1.00 | 180.90 | A | C |
| ATOM | 3622 | C | THR | C | 115 | −12.397 | 24.509 | −32.623 | 1.00 | 214.87 | A | C |
| ATOM | 3623 | O | THR | C | 115 | −12.367 | 25.744 | −32.640 | 1.00 | 232.79 | A | O |
| ATOM | 3624 | N | VAL | C | 116 | −12.680 | 23.770 | −33.699 | 1.00 | 243.08 | A | N |
| ATOM | 3625 | CA | VAL | C | 116 | −13.057 | 24.330 | −34.984 | 1.00 | 210.93 | A | C |
| ATOM | 3626 | CB | VAL | C | 116 | −13.231 | 23.224 | −36.059 | 1.00 | 212.69 | A | C |
| ATOM | 3627 | CG1 | VAL | C | 116 | −13.843 | 23.785 | −37.331 | 1.00 | 259.61 | A | C |
| ATOM | 3628 | CG2 | VAL | C | 116 | −11.905 | 22.526 | −36.340 | 1.00 | 271.62 | A | C |
| ATOM | 3629 | C | VAL | C | 116 | −14.355 | 25.113 | −34.827 | 1.00 | 221.92 | A | C |
| ATOM | 3630 | O | VAL | C | 116 | −14.377 | 26.314 | −35.088 | 1.00 | 232.31 | A | O |
| ATOM | 3631 | N | PRO | C | 117 | −15.438 | 24.437 | −34.385 | 1.00 | 250.29 | A | N |
| ATOM | 3632 | CA | PRO | C | 117 | −16.720 | 25.116 | −34.207 | 1.00 | 213.00 | A | C |
| ATOM | 3633 | CB | PRO | C | 117 | −17.686 | 23.975 | −33.873 | 1.00 | 257.90 | A | C |
| ATOM | 3634 | CG | PRO | C | 117 | −16.997 | 22.713 | −34.275 | 1.00 | 230.82 | A | C |
| ATOM | 3635 | CD | PRO | C | 117 | −15.543 | 22.996 | −34.086 | 1.00 | 211.84 | A | C |
| ATOM | 3636 | C | PRO | C | 117 | −16.661 | 26.129 | −33.067 | 1.00 | 206.66 | A | C |
| ATOM | 3637 | O | PRO | C | 117 | −17.412 | 27.096 | −33.063 | 1.00 | 206.70 | A | O |
| ATOM | 3638 | N | ALA | C | 118 | −15.767 | 25.903 | −32.106 | 1.00 | 199.20 | A | N |
| ATOM | 3639 | CA | ALA | C | 118 | −15.533 | 26.864 | −31.046 | 1.00 | 197.72 | A | C |
| ATOM | 3640 | CB | ALA | C | 118 | −14.565 | 26.302 | −30.037 | 1.00 | 221.76 | A | C |
| ATOM | 3641 | C | ALA | C | 118 | −14.993 | 28.156 | −31.619 | 1.00 | 221.74 | A | C |
| ATOM | 3642 | O | ALA | C | 118 | −15.446 | 29.228 | −31.236 | 1.00 | 237.30 | A | O |
| ATOM | 3643 | N | ALA | C | 119 | −14.041 | 28.051 | −32.545 | 1.00 | 233.26 | A | N |
| ATOM | 3644 | CA | ALA | C | 119 | −13.455 | 29.218 | −33.200 | 1.00 | 205.00 | A | C |
| ATOM | 3645 | CB | ALA | C | 119 | −12.289 | 28.801 | −34.075 | 1.00 | 213.52 | A | C |
| ATOM | 3646 | C | ALA | C | 119 | −14.504 | 29.975 | −34.019 | 1.00 | 229.88 | A | C |
| ATOM | 3647 | O | ALA | C | 119 | −14.666 | 31.185 | −33.846 | 1.00 | 236.77 | A | O |
| ATOM | 3648 | N | ILE | C | 120 | −15.209 | 29.249 | −34.894 | 1.00 | 245.93 | A | N |
| ATOM | 3649 | CA | ILE | C | 120 | −16.346 | 29.785 | −35.667 | 1.00 | 233.69 | A | C |
| ATOM | 3650 | CB | ILE | C | 120 | −17.180 | 28.656 | −36.349 | 1.00 | 203.42 | A | C |
| ATOM | 3651 | CG1 | ILE | C | 120 | −16.419 | 28.045 | −37.538 | 1.00 | 207.82 | A | C |
| ATOM | 3652 | CD1 | ILE | C | 120 | −16.992 | 26.743 | −38.067 | 1.00 | 221.44 | A | C |
| ATOM | 3653 | CG2 | ILE | C | 120 | −18.556 | 29.138 | −36.775 | 1.00 | 199.95 | A | C |
| ATOM | 3654 | C | ILE | C | 120 | −17.255 | 30.615 | −34.764 | 1.00 | 225.86 | A | C |
| ATOM | 3655 | O | ILE | C | 120 | −17.486 | 31.798 | −35.018 | 1.00 | 224.88 | A | O |
| ATOM | 3656 | N | LEU | C | 121 | −17.742 | 29.988 | −33.698 | 1.00 | 212.85 | A | N |
| ATOM | 3657 | CA | LEU | C | 121 | −18.734 | 30.598 | −32.843 | 1.00 | 204.98 | A | C |
| ATOM | 3658 | CB | LEU | C | 121 | −19.378 | 29.538 | −31.927 | 1.00 | 246.41 | A | C |
| ATOM | 3659 | CG | LEU | C | 121 | −20.812 | 29.768 | −31.388 | 1.00 | 235.18 | A | C |
| ATOM | 3660 | CD1 | LEU | C | 121 | −21.623 | 30.653 | −32.347 | 1.00 | 221.54 | A | C |
| ATOM | 3661 | CD2 | LEU | C | 121 | −21.537 | 28.465 | −31.028 | 1.00 | 323.97 | A | C |
| ATOM | 3662 | C | LEU | C | 121 | −18.150 | 31.757 | −32.040 | 1.00 | 230.45 | A | C |
| ATOM | 3663 | O | LEU | C | 121 | −18.874 | 32.695 | −31.694 | 1.00 | 224.21 | A | O |
| ATOM | 3664 | N | SER | C | 122 | −16.842 | 31.704 | −31.792 | 1.00 | 230.44 | A | N |
| ATOM | 3665 | CA | SER | C | 122 | −16.182 | 32.657 | −30.894 | 1.00 | 250.50 | A | C |
| ATOM | 3666 | CB | SER | C | 122 | −14.936 | 32.041 | −30.248 | 1.00 | 254.62 | A | C |
| ATOM | 3667 | OG | SER | C | 122 | −13.983 | 31.657 | −31.226 | 1.00 | 248.59 | A | O |
| ATOM | 3668 | C | SER | C | 122 | −15.830 | 33.964 | −31.599 | 1.00 | 247.13 | A | C |
| ATOM | 3669 | O | SER | C | 122 | −15.580 | 34.987 | −30.954 | 1.00 | 276.24 | A | O |
| ATOM | 3670 | N | HIS | C | 123 | −15.819 | 33.923 | −32.924 | 1.00 | 220.54 | A | N |
| ATOM | 3671 | CA | HIS | C | 123 | −15.548 | 35.108 | −33.710 | 1.00 | 244.71 | A | C |
| ATOM | 3672 | CB | HIS | C | 123 | −15.005 | 34.720 | −35.081 | 1.00 | 211.06 | A | C |
| ATOM | 3673 | CG | HIS | C | 123 | −14.727 | 35.897 | −35.965 | 1.00 | 211.70 | A | C |
| ATOM | 3674 | ND1 | HIS | C | 123 | −13.751 | 36.781 | −35.697 | 1.00 | 228.12 | A | N |
| ATOM | 3675 | CE1 | HIS | C | 123 | −13.741 | 37.734 | −36.643 | 1.00 | 204.76 | A | C |
| ATOM | 3676 | NE2 | HIS | C | 123 | −14.734 | 37.473 | −37.504 | 1.00 | 252.26 | A | N |
| ATOM | 3677 | CD2 | HIS | C | 123 | −15.363 | 36.346 | −37.118 | 1.00 | 219.69 | A | C |
| ATOM | 3678 | C | HIS | C | 123 | −16.761 | 35.997 | −33.848 | 1.00 | 237.37 | A | C |
| ATOM | 3679 | O | HIS | C | 123 | −16.628 | 37.189 | −34.133 | 1.00 | 237.97 | A | O |
| ATOM | 3680 | N | TYR | C | 124 | −17.951 | 35.429 | −33.646 | 1.00 | 255.88 | A | N |
| ATOM | 3681 | CA | TYR | C | 124 | −19.215 | 36.171 | −33.797 | 1.00 | 269.92 | A | C |
| ATOM | 3682 | CB | TYR | C | 124 | −20.275 | 35.333 | −34.536 | 1.00 | 224.44 | A | C |
| ATOM | 3683 | CG | TYR | C | 124 | −19.848 | 34.771 | −35.870 | 1.00 | 236.31 | A | C |
| ATOM | 3684 | CD1 | TYR | C | 124 | −18.911 | 35.443 | −36.665 | 1.00 | 232.29 | A | C |
| ATOM | 3685 | CE1 | TYR | C | 124 | −18.534 | 34.943 | −37.901 | 1.00 | 227.51 | A | C |
| ATOM | 3686 | CZ | TYR | C | 124 | −19.101 | 33.743 | −38.348 | 1.00 | 240.18 | A | C |
| ATOM | 3687 | OH | TYR | C | 124 | −18.729 | 33.217 | −39.558 | 1.00 | 227.93 | A | O |
| ATOM | 3688 | CE2 | TYR | C | 124 | −20.036 | 33.065 | −37.579 | 1.00 | 246.37 | A | C |
| ATOM | 3689 | CD2 | TYR | C | 124 | −20.410 | 33.584 | −36.352 | 1.00 | 243.85 | A | C |
| ATOM | 3690 | C | TYR | C | 124 | −19.769 | 36.651 | −32.447 | 1.00 | 245.11 | A | C |
| ATOM | 3691 | O | TYR | C | 124 | −20.973 | 36.604 | −32.193 | 1.00 | 263.81 | A | O |
| ATOM | 3692 | N | SER | C | 125 | −18.878 | 37.116 | −31.584 | 1.00 | 248.76 | A | N |
| ATOM | 3693 | CA | SER | C | 125 | −19.263 | 37.573 | −30.253 | 1.00 | 254.36 | A | C |
| ATOM | 3694 | CB | SER | C | 125 | −19.085 | 36.451 | −29.202 | 1.00 | 278.79 | A | C |
| ATOM | 3695 | OG | SER | C | 125 | −20.047 | 35.398 | −29.295 | 1.00 | 390.84 | A | O |
| ATOM | 3696 | C | SER | C | 125 | −18.414 | 38.784 | −29.854 | 1.00 | 282.16 | A | C |
| ATOM | 3697 | O | SER | C | 125 | −17.390 | 39.075 | −30.474 | 1.00 | 260.74 | A | O |
| ATOM | 3698 | N | ASP | C | 126 | −18.866 | 39.497 | −28.825 | 1.00 | 292.31 | A | N |
| ATOM | 3699 | CA | ASP | C | 126 | −17.993 | 40.350 | −28.021 | 1.00 | 273.35 | A | C |
| ATOM | 3700 | CB | ASP | C | 126 | −17.063 | 39.483 | −27.143 | 1.00 | 280.16 | A | C |
| ATOM | 3701 | CG | ASP | C | 126 | −17.804 | 38.516 | −26.230 | 1.00 | 288.25 | A | C |
| ATOM | 3702 | OD1 | ASP | C | 126 | −19.018 | 38.715 | −26.009 | 1.00 | 365.89 | A | O |
| ATOM | 3703 | OD2 | ASP | C | 126 | −17.151 | 37.557 | −25.742 | 1.00 | 191.86 | A | O |
| ATOM | 3704 | C | ASP | C | 126 | −17.123 | 41.325 | −28.821 | 1.00 | 242.69 | A | C |
| ATOM | 3705 | O | ASP | C | 126 | −15.924 | 41.423 | −28.573 | 1.00 | 253.09 | A | O |
| ATOM | 3706 | N | MET | C | 127 | −17.708 | 42.031 | −29.784 | 1.00 | 275.43 | A | N |
| ATOM | 3707 | CA | MET | C | 127 | −16.995 | 43.150 | −30.429 | 1.00 | 249.58 | A | C |
| ATOM | 3708 | CB | MET | C | 127 | −17.572 | 43.461 | −31.805 | 1.00 | 207.29 | A | C |
| ATOM | 3709 | CG | MET | C | 127 | −17.468 | 42.281 | −32.749 | 1.00 | 208.98 | A | C |
| ATOM | 3710 | SD | MET | C | 127 | −18.897 | 42.143 | −33.839 | 1.00 | 308.38 | A | S |
| ATOM | 3711 | CE | MET | C | 127 | −19.064 | 40.374 | −34.032 | 1.00 | 282.12 | A | C |
| ATOM | 3712 | C | MET | C | 127 | −17.051 | 44.359 | −29.495 | 1.00 | 243.26 | A | C |
| ATOM | 3713 | O | MET | C | 127 | −18.095 | 44.992 | −29.346 | 1.00 | 243.00 | A | O |
| ATOM | 3714 | N | LEU | C | 128 | −15.921 | 44.665 | −28.861 | 1.00 | 263.50 | A | N |
| ATOM | 3715 | CA | LEU | C | 128 | −15.930 | 45.483 | −27.647 | 1.00 | 259.94 | A | C |
| ATOM | 3716 | CB | LEU | C | 128 | −15.094 | 44.818 | −26.546 | 1.00 | 250.71 | A | C |
| ATOM | 3717 | CG | LEU | C | 128 | −15.546 | 43.402 | −26.159 | 1.00 | 188.54 | A | C |
| ATOM | 3718 | CD1 | LEU | C | 128 | −14.624 | 42.817 | −25.137 | 1.00 | 217.42 | A | C |
| ATOM | 3719 | CD2 | LEU | C | 128 | −16.985 | 43.353 | −25.675 | 1.00 | 203.63 | A | C |
| ATOM | 3720 | C | LEU | C | 128 | −15.532 | 46.935 | −27.856 | 1.00 | 273.01 | A | C |
| ATOM | 3721 | O | LEU | C | 128 | −16.143 | 47.837 | −27.267 | 1.00 | 293.66 | A | O |
| ATOM | 3722 | N | ASP | C | 129 | −14.524 | 47.164 | −28.698 | 1.00 | 294.34 | A | N |
| ATOM | 3723 | CA | ASP | C | 129 | −14.161 | 48.519 | −29.137 | 1.00 | 302.28 | A | C |
| ATOM | 3724 | CB | ASP | C | 129 | −15.413 | 49.340 | −29.534 | 1.00 | 305.21 | A | C |
| ATOM | 3725 | CG | ASP | C | 129 | −16.211 | 48.728 | −30.703 | 1.00 | 282.68 | A | C |
| ATOM | 3726 | OD1 | ASP | C | 129 | −15.680 | 47.873 | −31.448 | 1.00 | 306.96 | A | O |
| ATOM | 3727 | OD2 | ASP | C | 129 | −17.388 | 49.125 | −30.881 | 1.00 | 280.00 | A | O |
| ATOM | 3728 | C | ASP | C | 129 | −13.381 | 49.305 | −28.074 | 1.00 | 304.87 | A | C |
| ATOM | 3729 | O | ASP | C | 129 | −12.975 | 50.443 | −28.315 | 1.00 | 300.16 | A | O |
| ATOM | 3730 | N | ASP | C | 130 | −13.162 | 48.704 | −26.908 | 1.00 | 293.23 | A | N |
| ATOM | 3731 | CA | ASP | C | 130 | −12.759 | 49.467 | −25.727 | 1.00 | 292.71 | A | C |
| ATOM | 3732 | CB | ASP | C | 130 | −13.787 | 49.272 | −24.612 | 1.00 | 295.45 | A | C |
| ATOM | 3733 | CG | ASP | C | 130 | −15.073 | 50.044 | −24.864 | 1.00 | 276.70 | A | C |
| ATOM | 3734 | OD1 | ASP | C | 130 | −15.117 | 50.869 | −25.801 | 1.00 | 285.22 | A | O |
| ATOM | 3735 | OD2 | ASP | C | 130 | −16.046 | 49.830 | −24.116 | 1.00 | 306.01 | A | O |
| ATOM | 3736 | C | ASP | C | 130 | −11.377 | 49.153 | −25.201 | 1.00 | 293.87 | A | C |
| ATOM | 3737 | O | ASP | C | 130 | −11.037 | 49.519 | −24.072 | 1.00 | 283.80 | A | O |
| ATOM | 3738 | N | HIS | C | 131 | −10.571 | 48.503 | −26.028 | 1.00 | 304.31 | A | N |
| ATOM | 3739 | CA | HIS | C | 131 | −9.318 | 47.925 | −25.569 | 1.00 | 305.84 | A | C |
| ATOM | 3740 | CB | HIS | C | 131 | −9.162 | 46.524 | −26.138 | 1.00 | 260.98 | A | C |
| ATOM | 3741 | CG | HIS | C | 131 | −10.290 | 45.592 | −25.775 | 1.00 | 254.37 | A | C |
| ATOM | 3742 | ND1 | HIS | C | 131 | −11.591 | 45.962 | −25.844 | 1.00 | 244.72 | A | N |
| ATOM | 3743 | CE1 | HIS | C | 131 | −12.359 | 44.934 | −25.453 | 1.00 | 253.08 | A | C |
| ATOM | 3744 | NE2 | HIS | C | 131 | −11.557 | 43.901 | −25.148 | 1.00 | 252.33 | A | N |
| ATOM | 3745 | CD2 | HIS | C | 131 | −10.278 | 44.270 | −25.341 | 1.00 | 253.70 | A | C |
| ATOM | 3746 | C | HIS | C | 131 | −8.120 | 48.753 | −25.914 | 1.00 | 314.72 | A | C |
| ATOM | 3747 | O | HIS | C | 131 | −8.085 | 49.413 | −26.962 | 1.00 | 284.67 | A | O |
| ATOM | 3748 | N | LYS | C | 132 | −7.126 | 48.730 | −25.028 | 1.00 | 330.91 | A | N |
| ATOM | 3749 | CA | LYS | C | 132 | −5.787 | 49.171 | −25.383 | 1.00 | 316.39 | A | C |
| ATOM | 3750 | CB | LYS | C | 132 | −4.993 | 49.657 | −24.159 | 1.00 | 260.92 | A | C |
| ATOM | 3751 | CG | LYS | C | 132 | −5.028 | 51.176 | −23.994 | 1.00 | 242.89 | A | C |
| ATOM | 3752 | CD | LYS | C | 132 | −4.248 | 51.660 | −22.778 | 1.00 | 200.35 | A | C |
| ATOM | 3753 | CE | LYS | C | 132 | −3.470 | 52.946 | −23.059 | 1.00 | 193.42 | A | C |
| ATOM | 3754 | NZ | LYS | C | 132 | −4.254 | 54.097 | −23.607 | 1.00 | 177.56 | A | N |
| ATOM | 3755 | C | LYS | C | 132 | −5.087 | 48.047 | −26.166 | 1.00 | 331.95 | A | C |
| ATOM | 3756 | O | LYS | C | 132 | −3.944 | 48.204 | −26.609 | 1.00 | 362.61 | A | O |
| ATOM | 3757 | N | VAL | C | 133 | −5.793 | 46.927 | −26.360 | 1.00 | 301.82 | A | N |
| ATOM | 3758 | CA | VAL | C | 133 | −5.369 | 45.900 | −27.321 | 1.00 | 311.38 | A | C |
| ATOM | 3759 | CB | VAL | C | 133 | −6.417 | 44.761 | −27.482 | 1.00 | 296.74 | A | C |
| ATOM | 3760 | CG1 | VAL | C | 133 | −7.388 | 45.039 | −28.623 | 1.00 | 277.87 | A | C |
| ATOM | 3761 | CG2 | VAL | C | 133 | −5.725 | 43.425 | −27.698 | 1.00 | 266.95 | A | C |
| ATOM | 3762 | C | VAL | C | 133 | −5.002 | 46.594 | −28.647 | 1.00 | 330.77 | A | C |
| ATOM | 3763 | O | VAL | C | 133 | −5.495 | 47.690 | −28.949 | 1.00 | 302.37 | A | O |
| ATOM | 3764 | N | LEU | C | 134 | −4.135 | 45.954 | −29.425 | 1.00 | 299.44 | A | N |
| ATOM | 3765 | CA | LEU | C | 134 | −3.301 | 46.661 | −30.410 | 1.00 | 312.37 | A | C |
| ATOM | 3766 | CB | LEU | C | 134 | −2.145 | 45.763 | −30.876 | 1.00 | 324.21 | A | C |
| ATOM | 3767 | CG | LEU | C | 134 | −1.102 | 45.342 | −29.831 | 1.00 | 299.95 | A | C |
| ATOM | 3768 | CD1 | LEU | C | 134 | −1.693 | 44.486 | −28.713 | 1.00 | 261.53 | A | C |
| ATOM | 3769 | CD2 | LEU | C | 134 | 0.036 | 44.599 | −30.513 | 1.00 | 314.81 | A | C |
| ATOM | 3770 | C | LEU | C | 134 | −4.028 | 47.314 | −31.599 | 1.00 | 326.71 | A | C |
| ATOM | 3771 | O | LEU | C | 134 | −5.220 | 47.068 | −31.823 | 1.00 | 298.59 | A | O |
| ATOM | 3772 | N | GLY | C | 135 | −3.289 | 48.139 | −32.348 | 1.00 | 297.62 | A | N |
| ATOM | 3773 | CA | GLY | C | 135 | −3.844 | 49.010 | −33.400 | 1.00 | 274.12 | A | C |
| ATOM | 3774 | C | GLY | C | 135 | −4.079 | 48.378 | −34.758 | 1.00 | 301.24 | A | C |
| ATOM | 3775 | O | GLY | C | 135 | −4.540 | 49.050 | −35.690 | 1.00 | 310.22 | A | O |
| ATOM | 3776 | N | ILE | C | 136 | −3.742 | 47.094 | −34.876 | 1.00 | 357.42 | A | N |
| ATOM | 3777 | CA | ILE | C | 136 | −4.129 | 46.305 | −36.047 | 1.00 | 348.08 | A | C |
| ATOM | 3778 | CB | ILE | C | 136 | −3.042 | 45.300 | −36.498 | 1.00 | 319.99 | A | C |
| ATOM | 3779 | CG1 | ILE | C | 136 | −1.641 | 45.920 | −36.404 | 1.00 | 301.42 | A | C |
| ATOM | 3780 | CD1 | ILE | C | 136 | −0.523 | 44.912 | −36.228 | 1.00 | 219.98 | A | C |
| ATOM | 3781 | CG2 | ILE | C | 136 | −3.329 | 44.838 | −37.926 | 1.00 | 308.33 | A | C |
| ATOM | 3782 | C | ILE | C | 136 | −5.429 | 45.554 | −35.737 | 1.00 | 334.25 | A | C |
| ATOM | 3783 | O | ILE | C | 136 | −5.408 | 44.441 | −35.208 | 1.00 | 315.98 | A | O |
| ATOM | 3784 | N | THR | C | 137 | −6.560 | 46.178 | −36.065 | 1.00 | 350.09 | A | N |
| ATOM | 3785 | CA | THR | C | 137 | −7.900 | 45.591 | −35.856 | 1.00 | 355.19 | A | C |
| ATOM | 3786 | CB | THR | C | 137 | −8.336 | 44.707 | −37.049 | 1.00 | 339.24 | A | C |
| ATOM | 3787 | OG1 | THR | C | 137 | −7.302 | 43.758 | −37.356 | 1.00 | 348.82 | A | O |
| ATOM | 3788 | CG2 | THR | C | 137 | −8.637 | 45.577 | −38.289 | 1.00 | 314.46 | A | C |
| ATOM | 3789 | C | THR | C | 137 | −8.072 | 44.858 | −34.504 | 1.00 | 315.05 | A | C |
| ATOM | 3790 | O | THR | C | 137 | −8.045 | 45.500 | −33.456 | 1.00 | 278.38 | A | O |
| ATOM | 3791 | N | GLU | C | 138 | −8.237 | 43.536 | −34.532 | 1.00 | 265.60 | A | N |
| ATOM | 3792 | CA | GLU | C | 138 | −8.384 | 42.727 | −33.302 | 1.00 | 265.59 | A | C |
| ATOM | 3793 | CB | GLU | C | 138 | −7.313 | 43.087 | −32.275 | 1.00 | 278.32 | A | C |
| ATOM | 3794 | CG | GLU | C | 138 | −5.912 | 42.721 | −32.706 | 1.00 | 297.30 | A | C |
| ATOM | 3795 | CD | GLU | C | 138 | −4.871 | 43.596 | −32.049 | 1.00 | 290.14 | A | C |
| ATOM | 3796 | OE1 | GLU | C | 138 | −4.738 | 43.511 | −30.807 | 1.00 | 285.12 | A | O |
| ATOM | 3797 | OE2 | GLU | C | 138 | −4.190 | 44.357 | −32.771 | 1.00 | 286.26 | A | O |
| ATOM | 3798 | C | GLU | C | 138 | −9.768 | 42.776 | −32.641 | 1.00 | 283.19 | A | C |
| ATOM | 3799 | O | GLU | C | 138 | −9.888 | 42.796 | −31.409 | 1.00 | 269.02 | A | O |
| ATOM | 3800 | N | GLY | C | 139 | −10.807 | 42.797 | −33.468 | 1.00 | 306.49 | A | N |
| ATOM | 3801 | CA | GLY | C | 139 | −12.167 | 42.592 | −32.984 | 1.00 | 293.18 | A | C |
| ATOM | 3802 | C | GLY | C | 139 | −12.318 | 41.140 | −32.575 | 1.00 | 290.92 | A | C |
| ATOM | 3803 | O | GLY | C | 139 | −12.524 | 40.270 | −33.418 | 1.00 | 279.32 | A | O |
| ATOM | 3804 | N | ASP | C | 140 | −12.178 | 40.887 | −31.280 | 1.00 | 313.83 | A | N |
| ATOM | 3805 | CA | ASP | C | 140 | −12.376 | 39.549 | −30.691 | 1.00 | 306.36 | A | C |
| ATOM | 3806 | CB | ASP | C | 140 | −13.871 | 39.232 | −30.535 | 1.00 | 292.39 | A | C |
| ATOM | 3807 | CG | ASP | C | 140 | −14.115 | 37.995 | −29.691 | 1.00 | 257.16 | A | C |
| ATOM | 3808 | OD1 | ASP | C | 140 | −13.805 | 38.028 | −28.483 | 1.00 | 248.36 | A | O |
| ATOM | 3809 | OD2 | ASP | C | 140 | −14.608 | 36.983 | −30.228 | 1.00 | 248.20 | A | O |
| ATOM | 3810 | C | ASP | C | 140 | −11.661 | 38.382 | −31.403 | 1.00 | 318.88 | A | C |
| ATOM | 3811 | O | ASP | C | 140 | −12.302 | 37.543 | −32.061 | 1.00 | 278.82 | A | O |
| ATOM | 3812 | N | TRP | C | 141 | −10.340 | 38.320 | −31.240 | 1.00 | 284.84 | A | N |
| ATOM | 3813 | CA | TRP | C | 141 | −9.530 | 37.238 | −31.830 | 1.00 | 270.46 | A | C |
| ATOM | 3814 | CB | TRP | C | 141 | −8.131 | 37.734 | −32.233 | 1.00 | 276.85 | A | C |
| ATOM | 3815 | CG | TRP | C | 141 | −7.405 | 38.552 | −31.184 | 1.00 | 293.89 | A | C |
| ATOM | 3816 | CD1 | TRP | C | 141 | −7.847 | 39.717 | −30.551 | 1.00 | 318.98 | A | C |
| ATOM | 3817 | NE1 | TRP | C | 141 | −6.904 | 40.193 | −29.671 | 1.00 | 309.67 | A | N |
| ATOM | 3818 | CE2 | TRP | C | 141 | −5.803 | 39.411 | −29.675 | 1.00 | 324.79 | A | C |
| ATOM | 3819 | CD2 | TRP | C | 141 | −6.047 | 38.327 | −30.647 | 1.00 | 339.63 | A | C |
| ATOM | 3820 | CE3 | TRP | C | 141 | −5.053 | 37.373 | −30.856 | 1.00 | 324.54 | A | C |
| ATOM | 3821 | CZ3 | TRP | C | 141 | −3.852 | 37.488 | −30.130 | 1.00 | 303.11 | A | C |
| ATOM | 3822 | CH2 | TRP | C | 141 | −3.639 | 38.533 | −29.209 | 1.00 | 284.69 | A | C |
| ATOM | 3823 | CZ2 | TRP | C | 141 | −4.608 | 39.515 | −28.968 | 1.00 | 259.16 | A | C |
| ATOM | 3824 | C | TRP | C | 141 | −9.482 | 35.957 | −31.026 | 1.00 | 214.89 | A | C |
| ATOM | 3825 | O | TRP | C | 141 | −8.657 | 35.069 | −31.295 | 1.00 | 214.84 | A | O |
| ATOM | 3826 | N | TRP | C | 142 | −10.386 | 35.842 | −30.052 | 1.00 | 238.20 | A | N |
| ATOM | 3827 | CA | TRP | C | 142 | −10.712 | 34.564 | −29.427 | 1.00 | 209.91 | A | C |
| ATOM | 3828 | CB | TRP | C | 142 | −12.054 | 34.651 | −28.700 | 1.00 | 223.65 | A | C |
| ATOM | 3829 | CG | TRP | C | 142 | −11.942 | 34.806 | −27.197 | 1.00 | 249.95 | A | C |
| ATOM | 3830 | CD1 | TRP | C | 142 | −11.125 | 35.695 | −26.493 | 1.00 | 212.10 | A | C |
| ATOM | 3831 | NE1 | TRP | C | 142 | −11.294 | 35.550 | −25.139 | 1.00 | 233.88 | A | N |
| ATOM | 3832 | CE2 | TRP | C | 142 | −12.207 | 34.575 | −24.869 | 1.00 | 246.24 | A | C |
| ATOM | 3833 | CD2 | TRP | C | 142 | −12.681 | 34.056 | −26.154 | 1.00 | 272.97 | A | C |
| ATOM | 3834 | CE3 | TRP | C | 142 | −13.647 | 33.036 | −26.160 | 1.00 | 251.51 | A | C |
| ATOM | 3835 | CZ3 | TRP | C | 142 | −14.136 | 32.551 | −24.932 | 1.00 | 246.03 | A | C |
| ATOM | 3836 | CH2 | TRP | C | 142 | −13.670 | 33.061 | −23.709 | 1.00 | 248.84 | A | C |
| ATOM | 3837 | CZ2 | TRP | C | 142 | −12.694 | 34.086 | −23.657 | 1.00 | 260.78 | A | C |
| ATOM | 3838 | C | TRP | C | 142 | −10.762 | 33.446 | −30.427 | 1.00 | 199.94 | A | C |
| ATOM | 3839 | O | TRP | C | 142 | −10.366 | 32.319 | −30.144 | 1.00 | 195.02 | A | O |
| ATOM | 3840 | N | ALA | C | 143 | −11.256 | 33.763 | −31.613 | 1.00 | 207.43 | A | N |
| ATOM | 3841 | CA | ALA | C | 143 | −11.399 | 32.799 | −32.698 | 1.00 | 219.82 | A | C |
| ATOM | 3842 | CB | ALA | C | 143 | −11.861 | 33.492 | −33.976 | 1.00 | 209.56 | A | C |
| ATOM | 3843 | C | ALA | C | 143 | −10.106 | 32.041 | −32.946 | 1.00 | 213.51 | A | C |
| ATOM | 3844 | O | ALA | C | 143 | −10.102 | 30.808 | −32.953 | 1.00 | 188.31 | A | O |
| ATOM | 3845 | N | ILE | C | 144 | −9.016 | 32.788 | −33.116 | 1.00 | 252.64 | A | N |
| ATOM | 3846 | CA | ILE | C | 144 | −7.733 | 32.227 | −33.554 | 1.00 | 238.58 | A | C |
| ATOM | 3847 | CB | ILE | C | 144 | −6.827 | 33.302 | −34.210 | 1.00 | 242.64 | A | C |
| ATOM | 3848 | CG1 | ILE | C | 144 | −6.376 | 34.351 | −33.175 | 1.00 | 262.71 | A | C |
| ATOM | 3849 | CD1 | ILE | C | 144 | −5.741 | 35.596 | −33.753 | 1.00 | 255.28 | A | C |
| ATOM | 3850 | CG2 | ILE | C | 144 | −7.530 | 33.936 | −35.413 | 1.00 | 206.72 | A | C |
| ATOM | 3851 | C | ILE | C | 144 | −7.006 | 31.514 | −32.422 | 1.00 | 202.63 | A | C |
| ATOM | 3852 | O | ILE | C | 144 | −6.131 | 30.681 | −32.667 | 1.00 | 221.70 | A | O |
| ATOM | 3853 | N | ILE | C | 145 | −7.386 | 31.851 | −31.191 | 1.00 | 216.75 | A | N |
| ATOM | 3854 | CA | ILE | C | 145 | −6.933 | 31.132 | −30.003 | 1.00 | 197.67 | A | C |
| ATOM | 3855 | CB | ILE | C | 145 | −7.473 | 31.775 | −28.714 | 1.00 | 168.38 | A | C |
| ATOM | 3856 | CG1 | ILE | C | 145 | −6.679 | 33.025 | −28.377 | 1.00 | 188.12 | A | C |
| ATOM | 3857 | CD1 | ILE | C | 145 | −7.421 | 33.972 | −27.460 | 1.00 | 208.62 | A | C |
| ATOM | 3858 | CG2 | ILE | C | 145 | −7.414 | 30.811 | −27.574 | 1.00 | 150.68 | A | C |
| ATOM | 3859 | C | ILE | C | 145 | −7.390 | 29.685 | −30.062 | 1.00 | 209.15 | A | C |
| ATOM | 3860 | O | ILE | C | 145 | −6.571 | 28.770 | −29.902 | 1.00 | 207.37 | A | O |
| ATOM | 3861 | N | TRP | C | 146 | −8.694 | 29.483 | −30.270 | 1.00 | 225.23 | A | N |
| ATOM | 3862 | CA | TRP | C | 146 | −9.292 | 28.145 | −30.307 | 1.00 | 234.44 | A | C |
| ATOM | 3863 | CB | TRP | C | 146 | −10.782 | 28.232 | −30.622 | 1.00 | 232.75 | A | C |
| ATOM | 3864 | CG | TRP | C | 146 | −11.657 | 28.511 | −29.423 | 1.00 | 220.52 | A | C |
| ATOM | 3865 | CD1 | TRP | C | 146 | −12.357 | 29.677 | −29.129 | 1.00 | 243.26 | A | C |
| ATOM | 3866 | NE1 | TRP | C | 146 | −13.049 | 29.556 | −27.948 | 1.00 | 243.02 | A | N |
| ATOM | 3867 | CE2 | TRP | C | 146 | −12.853 | 28.339 | −27.408 | 1.00 | 241.94 | A | C |
| ATOM | 3868 | CD2 | TRP | C | 146 | −11.953 | 27.603 | −28.313 | 1.00 | 247.74 | A | C |
| ATOM | 3869 | CE3 | TRP | C | 146 | −11.580 | 26.304 | −27.985 | 1.00 | 250.52 | A | C |
| ATOM | 3870 | CZ3 | TRP | C | 146 | −12.080 | 25.749 | −26.803 | 1.00 | 281.98 | A | C |
| ATOM | 3871 | CH2 | TRP | C | 146 | −12.936 | 26.470 | −25.952 | 1.00 | 264.99 | A | C |
| ATOM | 3872 | CZ2 | TRP | C | 146 | −13.336 | 27.777 | −26.236 | 1.00 | 244.50 | A | C |
| ATOM | 3873 | C | TRP | C | 146 | −8.615 | 27.246 | −31.302 | 1.00 | 209.06 | A | C |
| ATOM | 3874 | O | TRP | C | 146 | −8.438 | 26.052 | −31.061 | 1.00 | 194.02 | A | O |
| ATOM | 3875 | N | LEU | C | 147 | −8.233 | 27.824 | −32.434 | 1.00 | 246.48 | A | N |
| ATOM | 3876 | CA | LEU | C | 147 | −7.533 | 27.088 | −33.464 | 1.00 | 249.01 | A | C |
| ATOM | 3877 | CB | LEU | C | 147 | −7.620 | 27.820 | −34.800 | 1.00 | 246.42 | A | C |
| ATOM | 3878 | CG | LEU | C | 147 | −9.048 | 28.012 | −35.313 | 1.00 | 257.68 | A | C |
| ATOM | 3879 | CD1 | LEU | C | 147 | −9.029 | 28.909 | −36.535 | 1.00 | 272.11 | A | C |
| ATOM | 3880 | CD2 | LEU | C | 147 | −9.749 | 26.692 | −35.613 | 1.00 | 268.03 | A | C |
| ATOM | 3881 | C | LEU | C | 147 | −6.084 | 26.857 | −33.065 | 1.00 | 241.79 | A | C |
| ATOM | 3882 | O | LEU | C | 147 | −5.496 | 25.846 | −33.452 | 1.00 | 252.56 | A | O |
| ATOM | 3883 | N | ALA | C | 148 | −5.523 | 27.787 | −32.288 | 1.00 | 234.10 | A | N |
| ATOM | 3884 | CA | ALA | C | 148 | −4.157 | 27.655 | −31.760 | 1.00 | 215.23 | A | C |
| ATOM | 3885 | CB | ALA | C | 148 | −3.681 | 28.966 | −31.163 | 1.00 | 200.24 | A | C |
| ATOM | 3886 | C | ALA | C | 148 | −4.068 | 26.558 | −30.716 | 1.00 | 215.75 | A | C |
| ATOM | 3887 | O | ALA | C | 148 | −3.130 | 25.757 | −30.719 | 1.00 | 224.46 | A | O |
| ATOM | 3888 | N | TRP | C | 149 | −5.045 | 26.537 | −29.812 | 1.00 | 203.86 | A | N |
| ATOM | 3889 | CA | TRP | C | 149 | −5.143 | 25.493 | −28.804 | 1.00 | 188.81 | A | C |
| ATOM | 3890 | CB | TRP | C | 149 | −6.192 | 25.824 | −27.754 | 1.00 | 194.91 | A | C |
| ATOM | 3891 | CG | TRP | C | 149 | −5.784 | 26.931 | −26.816 | 1.00 | 216.34 | A | C |
| ATOM | 3892 | CD1 | TRP | C | 149 | −4.506 | 27.450 | −26.621 | 1.00 | 226.76 | A | C |
| ATOM | 3893 | NE1 | TRP | C | 149 | −4.516 | 28.441 | −25.671 | 1.00 | 239.60 | A | N |
| ATOM | 3894 | CE2 | TRP | C | 149 | −5.764 | 28.634 | −25.189 | 1.00 | 227.98 | A | C |
| ATOM | 3895 | CD2 | TRP | C | 149 | −6.645 | 27.680 | −25.884 | 1.00 | 228.08 | A | C |
| ATOM | 3896 | CE3 | TRP | C | 149 | −8.003 | 27.663 | −25.571 | 1.00 | 183.26 | A | C |
| ATOM | 3897 | CZ3 | TRP | C | 149 | −8.476 | 28.566 | −24.599 | 1.00 | 197.96 | A | C |
| ATOM | 3898 | CH2 | TRP | C | 149 | −7.615 | 29.474 | −23.946 | 1.00 | 221.41 | A | C |
| ATOM | 3899 | CZ2 | TRP | C | 149 | −6.246 | 29.526 | −24.233 | 1.00 | 234.95 | A | C |
| ATOM | 3900 | C | TRP | C | 149 | −5.443 | 24.178 | −29.438 | 1.00 | 220.52 | A | C |
| ATOM | 3901 | O | TRP | C | 149 | −4.833 | 23.173 | −29.081 | 1.00 | 233.48 | A | O |
| ATOM | 3902 | N | GLY | C | 150 | −6.385 | 24.170 | −30.380 | 1.00 | 219.22 | A | N |
| ATOM | 3903 | CA | GLY | C | 150 | −6.671 | 22.981 | −31.178 | 1.00 | 231.07 | A | C |
| ATOM | 3904 | C | GLY | C | 150 | −5.405 | 22.439 | −31.815 | 1.00 | 229.54 | A | C |
| ATOM | 3905 | O | GLY | C | 150 | −5.266 | 21.227 | −32.018 | 1.00 | 238.88 | A | O |
| ATOM | 3906 | N | VAL | C | 151 | −4.474 | 23.346 | −32.113 | 1.00 | 246.61 | A | N |
| ATOM | 3907 | CA | VAL | C | 151 | −3.217 | 22.990 | −32.765 | 1.00 | 245.36 | A | C |
| ATOM | 3908 | CB | VAL | C | 151 | −2.497 | 24.242 | −33.323 | 1.00 | 247.84 | A | C |
| ATOM | 3909 | CG1 | VAL | C | 151 | −0.991 | 24.097 | −33.222 | 1.00 | 260.48 | A | C |
| ATOM | 3910 | CG2 | VAL | C | 151 | −2.918 | 24.531 | −34.761 | 1.00 | 250.84 | A | C |
| ATOM | 3911 | C | VAL | C | 151 | −2.307 | 22.194 | −31.828 | 1.00 | 230.82 | A | C |
| ATOM | 3912 | O | VAL | C | 151 | −1.907 | 21.069 | −32.155 | 1.00 | 238.45 | A | O |
| ATOM | 3913 | N | LEU | C | 152 | −1.988 | 22.774 | −30.670 | 1.00 | 232.47 | A | N |
| ATOM | 3914 | CA | LEU | C | 152 | −1.248 | 22.051 | −29.637 | 1.00 | 208.90 | A | C |
| ATOM | 3915 | CB | LEU | C | 152 | −1.249 | 22.815 | −28.315 | 1.00 | 191.11 | A | C |
| ATOM | 3916 | CG | LEU | C | 152 | 0.089 | 23.416 | −27.899 | 1.00 | 195.72 | A | C |
| ATOM | 3917 | CD1 | LEU | C | 152 | −0.101 | 24.180 | −26.595 | 1.00 | 184.36 | A | C |
| ATOM | 3918 | CD2 | LEU | C | 152 | 1.168 | 22.353 | −27.775 | 1.00 | 214.69 | A | C |
| ATOM | 3919 | C | LEU | C | 152 | −1.796 | 20.651 | −29.414 | 1.00 | 211.29 | A | C |
| ATOM | 3920 | O | LEU | C | 152 | −1.078 | 19.675 | −29.582 | 1.00 | 225.41 | A | O |
| ATOM | 3921 | N | TRP | C | 153 | −3.078 | 20.569 | −29.066 | 1.00 | 213.02 | A | N |
| ATOM | 3922 | CA | TRP | C | 153 | −3.727 | 19.299 | −28.738 | 1.00 | 229.71 | A | C |
| ATOM | 3923 | CB | TRP | C | 153 | −5.194 | 19.527 | −28.388 | 1.00 | 216.37 | A | C |
| ATOM | 3924 | CG | TRP | C | 153 | −5.391 | 19.830 | −26.925 | 1.00 | 216.03 | A | C |
| ATOM | 3925 | CD1 | TRP | C | 153 | −6.155 | 19.118 | −26.002 | 1.00 | 192.90 | A | C |
| ATOM | 3926 | NE1 | TRP | C | 153 | −6.064 | 19.684 | −24.757 | 1.00 | 167.47 | A | N |
| ATOM | 3927 | CE2 | TRP | C | 153 | −5.258 | 20.763 | −24.774 | 1.00 | 196.85 | A | C |
| ATOM | 3928 | CD2 | TRP | C | 153 | −4.774 | 20.915 | −26.147 | 1.00 | 213.17 | A | C |
| ATOM | 3929 | CE3 | TRP | C | 153 | −3.906 | 21.957 | −26.442 | 1.00 | 187.32 | A | C |
| ATOM | 3930 | CZ3 | TRP | C | 153 | −3.537 | 22.824 | −25.408 | 1.00 | 181.90 | A | C |
| ATOM | 3931 | CH2 | TRP | C | 153 | −4.018 | 22.658 | −24.099 | 1.00 | 191.10 | A | C |
| ATOM | 3932 | CZ2 | TRP | C | 153 | −4.882 | 21.630 | −23.763 | 1.00 | 195.33 | A | C |
| ATOM | 3933 | C | TRP | C | 153 | −3.596 | 18.295 | −29.832 | 1.00 | 209.39 | A | C |
| ATOM | 3934 | O | TRP | C | 153 | −3.354 | 17.118 | −29.573 | 1.00 | 206.15 | A | O |
| ATOM | 3935 | N | LEU | C | 154 | −3.726 | 18.755 | −31.065 | 1.00 | 214.42 | A | N |
| ATOM | 3936 | CA | LEU | C | 154 | −3.620 | 17.880 | −32.214 | 1.00 | 235.15 | A | C |
| ATOM | 3937 | CB | LEU | C | 154 | −3.961 | 18.648 | −33.495 | 1.00 | 238.18 | A | C |
| ATOM | 3938 | CG | LEU | C | 154 | −4.264 | 17.816 | −34.739 | 1.00 | 214.59 | A | C |
| ATOM | 3939 | CD1 | LEU | C | 154 | −5.112 | 16.603 | −34.401 | 1.00 | 213.58 | A | C |
| ATOM | 3940 | CD2 | LEU | C | 154 | −4.966 | 18.678 | −35.750 | 1.00 | 253.53 | A | C |
| ATOM | 3941 | C | LEU | C | 154 | −2.235 | 17.237 | −32.307 | 1.00 | 216.51 | A | C |
| ATOM | 3942 | O | LEU | C | 154 | −2.117 | 16.088 | −32.715 | 1.00 | 211.73 | A | O |
| ATOM | 3943 | N | THR | C | 155 | −1.201 | 17.976 | −31.908 | 1.00 | 206.11 | A | N |
| ATOM | 3944 | CA | THR | C | 155 | 0.177 | 17.498 | −32.009 | 1.00 | 209.68 | A | C |
| ATOM | 3945 | CB | THR | C | 155 | 1.189 | 18.509 | −31.443 | 1.00 | 237.69 | A | C |
| ATOM | 3946 | OG1 | THR | C | 155 | 0.998 | 18.631 | −30.032 | 1.00 | 249.41 | A | O |
| ATOM | 3947 | CG2 | THR | C | 155 | 1.041 | 19.887 | −32.121 | 1.00 | 223.27 | A | C |
| ATOM | 3948 | C | THR | C | 155 | 0.334 | 16.171 | −31.288 | 1.00 | 207.35 | A | C |
| ATOM | 3949 | O | THR | C | 155 | 0.884 | 15.212 | −31.805 | 1.00 | 219.91 | A | O |
| ATOM | 3950 | N | ALA | C | 156 | −0.183 | 16.113 | −30.080 | 1.00 | 207.07 | A | N |
| ATOM | 3951 | CA | ALA | C | 156 | −0.252 | 14.854 | −29.358 | 1.00 | 214.39 | A | C |
| ATOM | 3952 | CB | ALA | C | 156 | −1.042 | 15.029 | −28.067 | 1.00 | 215.22 | A | C |
| ATOM | 3953 | C | ALA | C | 156 | −0.861 | 13.749 | −30.236 | 1.00 | 215.89 | A | C |
| ATOM | 3954 | O | ALA | C | 156 | −0.440 | 12.598 | −30.155 | 1.00 | 224.10 | A | O |
| ATOM | 3955 | N | PHE | C | 157 | −1.815 | 14.106 | −31.097 | 1.00 | 202.12 | A | N |
| ATOM | 3956 | CA | PHE | C | 157 | −2.516 | 13.110 | −31.900 | 1.00 | 194.24 | A | C |
| ATOM | 3957 | CB | PHE | C | 157 | −3.770 | 13.677 | −32.558 | 1.00 | 198.39 | A | C |
| ATOM | 3958 | CG | PHE | C | 157 | −4.280 | 12.826 | −33.670 | 1.00 | 204.99 | A | C |
| ATOM | 3959 | CD1 | PHE | C | 157 | −4.950 | 11.653 | −33.401 | 1.00 | 204.95 | A | C |
| ATOM | 3960 | CE1 | PHE | C | 157 | −5.416 | 10.856 | −34.429 | 1.00 | 228.38 | A | C |
| ATOM | 3961 | CZ | PHE | C | 157 | −5.182 | 11.206 | −35.744 | 1.00 | 259.32 | A | C |
| ATOM | 3962 | CE2 | PHE | C | 157 | −4.488 | 12.368 | −36.024 | 1.00 | 254.25 | A | C |
| ATOM | 3963 | CD2 | PHE | C | 157 | −4.047 | 13.172 | −34.987 | 1.00 | 222.75 | A | C |
| ATOM | 3964 | C | PHE | C | 157 | −1.629 | 12.490 | −32.956 | 1.00 | 211.48 | A | C |
| ATOM | 3965 | O | PHE | C | 157 | −1.354 | 11.305 | −32.911 | 1.00 | 214.07 | A | O |
| ATOM | 3966 | N | ILE | C | 158 | −1.208 | 13.298 | −33.919 | 1.00 | 224.95 | A | N |
| ATOM | 3967 | CA | ILE | C | 158 | −0.363 | 12.832 | −35.011 | 1.00 | 239.78 | A | C |
| ATOM | 3968 | CB | ILE | C | 158 | 0.123 | 13.993 | −35.894 | 1.00 | 224.78 | A | C |
| ATOM | 3969 | CG1 | ILE | C | 158 | 0.665 | 15.131 | −35.032 | 1.00 | 222.43 | A | C |
| ATOM | 3970 | CD1 | ILE | C | 158 | 1.756 | 15.971 | −35.636 | 1.00 | 221.17 | A | C |
| ATOM | 3971 | CG2 | ILE | C | 158 | −1.018 | 14.484 | −36.760 | 1.00 | 249.16 | A | C |
| ATOM | 3972 | C | ILE | C | 158 | 0.838 | 12.064 | −34.487 | 1.00 | 251.36 | A | C |
| ATOM | 3973 | O | ILE | C | 158 | 1.070 | 10.937 | −34.913 | 1.00 | 221.87 | A | O |
| ATOM | 3974 | N | GLU | C | 159 | 1.569 | 12.678 | −33.548 | 1.00 | 249.08 | A | N |
| ATOM | 3975 | CA | GLU | C | 159 | 2.837 | 12.140 | −33.036 | 1.00 | 238.29 | A | C |
| ATOM | 3976 | CB | GLU | C | 159 | 3.483 | 13.057 | −31.997 | 1.00 | 261.81 | A | C |
| ATOM | 3977 | CG | GLU | C | 159 | 4.903 | 12.645 | −31.623 | 1.00 | 323.64 | A | C |
| ATOM | 3978 | CD | GLU | C | 159 | 5.757 | 13.827 | −31.195 | 1.00 | 332.51 | A | C |
| ATOM | 3979 | OE1 | GLU | C | 159 | 5.222 | 14.795 | −30.622 | 1.00 | 284.43 | A | O |
| ATOM | 3980 | OE2 | GLU | C | 159 | 6.970 | 13.818 | −31.457 | 1.00 | 300.69 | A | O |
| ATOM | 3981 | C | GLU | C | 159 | 2.686 | 10.796 | −32.422 | 1.00 | 224.36 | A | C |
| ATOM | 3982 | O | GLU | C | 159 | 3.519 | 9.914 | −32.617 | 1.00 | 252.19 | A | O |
| ATOM | 3983 | N | ASN | C | 160 | 1.595 | 10.650 | −31.699 | 1.00 | 197.06 | A | N |
| ATOM | 3984 | CA | ASN | C | 160 | 1.442 | 9.501 | −30.851 | 1.00 | 207.68 | A | C |
| ATOM | 3985 | CB | ASN | C | 160 | 0.893 | 9.902 | −29.477 | 1.00 | 226.17 | A | C |
| ATOM | 3986 | CG | ASN | C | 160 | 1.974 | 10.430 | −28.546 | 1.00 | 199.59 | A | C |
| ATOM | 3987 | OD1 | ASN | C | 160 | 3.051 | 9.846 | −28.419 | 1.00 | 208.33 | A | O |
| ATOM | 3988 | ND2 | ASN | C | 160 | 1.680 | 11.539 | −27.879 | 1.00 | 206.68 | A | N |
| ATOM | 3989 | C | ASN | C | 160 | 0.623 | 8.379 | −31.445 | 1.00 | 213.18 | A | C |
| ATOM | 3990 | O | ASN | C | 160 | 0.979 | 7.225 | −31.256 | 1.00 | 215.36 | A | O |
| ATOM | 3991 | N | ILE | C | 161 | −0.458 | 8.689 | −32.157 | 1.00 | 198.90 | A | N |
| ATOM | 3992 | CA | ILE | C | 161 | −1.453 | 7.643 | −32.402 | 1.00 | 226.85 | A | C |
| ATOM | 3993 | CB | ILE | C | 161 | −2.913 | 8.127 | −32.282 | 1.00 | 213.60 | A | C |
| ATOM | 3994 | CG1 | ILE | C | 161 | −3.846 | 6.912 | −32.235 | 1.00 | 233.66 | A | C |
| ATOM | 3995 | CD1 | ILE | C | 161 | −3.502 | 5.875 | −31.169 | 1.00 | 217.54 | A | C |
| ATOM | 3996 | CG2 | ILE | C | 161 | −3.272 | 9.040 | −33.426 | 1.00 | 174.44 | A | C |
| ATOM | 3997 | C | ILE | C | 161 | −1.254 | 6.766 | −33.642 | 1.00 | 233.98 | A | C |
| ATOM | 3998 | O | ILE | C | 161 | −1.355 | 5.539 | −33.555 | 1.00 | 248.02 | A | O |
| ATOM | 3999 | N | LEU | C | 162 | −1.003 | 7.388 | −34.785 | 1.00 | 222.57 | A | N |
| ATOM | 4000 | CA | LEU | C | 162 | −0.626 | 6.632 | −35.966 | 1.00 | 258.90 | A | C |
| ATOM | 4001 | CB | LEU | C | 162 | −1.780 | 5.765 | −36.498 | 1.00 | 277.71 | A | C |
| ATOM | 4002 | CG | LEU | C | 162 | −1.358 | 4.364 | −36.978 | 1.00 | 276.33 | A | C |
| ATOM | 4003 | CD1 | LEU | C | 162 | −0.511 | 3.641 | −35.930 | 1.00 | 219.85 | A | C |
| ATOM | 4004 | CD2 | LEU | C | 162 | −2.562 | 3.515 | −37.379 | 1.00 | 276.76 | A | C |
| ATOM | 4005 | C | LEU | C | 162 | −0.103 | 7.586 | −37.014 | 1.00 | 263.25 | A | C |
| ATOM | 4006 | O | LEU | C | 162 | −0.535 | 7.573 | −38.171 | 1.00 | 270.92 | A | O |
| ATOM | 4007 | N | LYS | C | 163 | 0.812 | 8.445 | −36.579 | 1.00 | 254.41 | A | N |
| ATOM | 4008 | CA | LYS | C | 163 | 1.639 | 9.185 | −37.513 | 1.00 | 255.98 | A | C |
| ATOM | 4009 | CB | LYS | C | 163 | 1.137 | 10.596 | −37.761 | 1.00 | 272.05 | A | C |
| ATOM | 4010 | CG | LYS | C | 163 | 2.149 | 11.372 | −38.588 | 1.00 | 307.06 | A | C |
| ATOM | 4011 | CD | LYS | C | 163 | 1.743 | 12.799 | −38.797 | 1.00 | 315.49 | A | C |
| ATOM | 4012 | CE | LYS | C | 163 | 2.523 | 13.443 | −39.949 | 1.00 | 318.57 | A | C |
| ATOM | 4013 | NZ | LYS | C | 163 | 1.717 | 14.067 | −41.075 | 1.00 | 362.02 | A | N |
| ATOM | 4014 | C | LYS | C | 163 | 3.124 | 9.248 | −37.173 | 1.00 | 280.37 | A | C |
| ATOM | 4015 | O | LYS | C | 163 | 3.936 | 9.360 | −38.082 | 1.00 | 316.15 | A | O |
| ATOM | 4016 | N | ILE | C | 164 | 3.473 | 9.263 | −35.899 | 1.00 | 254.33 | A | N |
| ATOM | 4017 | CA | ILE | C | 164 | 4.884 | 9.311 | −35.447 | 1.00 | 256.13 | A | C |
| ATOM | 4018 | CB | ILE | C | 164 | 5.510 | 7.896 | −35.367 | 1.00 | 259.83 | A | C |
| ATOM | 4019 | CG1 | ILE | C | 164 | 5.411 | 7.169 | −36.719 | 1.00 | 264.78 | A | C |
| ATOM | 4020 | CD1 | ILE | C | 164 | 6.341 | 5.979 | −36.856 | 1.00 | 293.79 | A | C |
| ATOM | 4021 | CG2 | ILE | C | 164 | 4.850 | 7.094 | −34.243 | 1.00 | 246.46 | A | C |
| ATOM | 4022 | C | ILE | C | 164 | 5.827 | 10.324 | −36.156 | 1.00 | 299.85 | A | C |
| ATOM | 4023 | O | ILE | C | 164 | 6.824 | 9.949 | −36.778 | 1.00 | 299.87 | A | O |
| ATOM | 4024 | N | PRO | C | 165 | 5.504 | 11.620 | −36.040 | 1.00 | 306.24 | A | N |
| ATOM | 4025 | CA | PRO | C | 165 | 6.321 | 12.714 | −36.505 | 1.00 | 299.79 | A | C |
| ATOM | 4026 | CB | PRO | C | 165 | 5.262 | 13.723 | −36.946 | 1.00 | 281.71 | A | C |
| ATOM | 4027 | CG | PRO | C | 165 | 4.236 | 13.561 | −35.884 | 1.00 | 265.35 | A | C |
| ATOM | 4028 | CD | PRO | C | 165 | 4.131 | 12.080 | −35.773 | 1.00 | 280.02 | A | C |
| ATOM | 4029 | C | PRO | C | 165 | 7.193 | 13.320 | −35.405 | 1.00 | 304.19 | A | C |
| ATOM | 4030 | O | PRO | C | 165 | 7.245 | 12.795 | −34.330 | 1.00 | 290.11 | A | O |
| ATOM | 4031 | N | LEU | C | 166 | 7.761 | 14.483 | −35.679 | 1.00 | 300.67 | A | N |
| ATOM | 4032 | CA | LEU | C | 166 | 8.827 | 15.133 | −34.956 | 1.00 | 294.19 | A | C |
| ATOM | 4033 | CB | LEU | C | 166 | 9.802 | 15.794 | −35.970 | 1.00 | 309.09 | A | C |
| ATOM | 4034 | CG | LEU | C | 166 | 9.831 | 15.509 | −37.494 | 1.00 | 306.41 | A | C |
| ATOM | 4035 | CD1 | LEU | C | 166 | 10.039 | 16.778 | −38.321 | 1.00 | 261.21 | A | C |
| ATOM | 4036 | CD2 | LEU | C | 166 | 10.876 | 14.459 | −37.819 | 1.00 | 330.78 | A | C |
| ATOM | 4037 | C | LEU | C | 166 | 8.274 | 16.228 | −34.030 | 1.00 | 323.16 | A | C |
| ATOM | 4038 | O | LEU | C | 166 | 7.171 | 16.772 | −34.230 | 1.00 | 299.32 | A | O |
| ATOM | 4039 | N | GLY | C | 167 | 9.086 | 16.610 | −33.065 | 1.00 | 297.75 | A | N |
| ATOM | 4040 | CA | GLY | C | 167 | 8.530 | 17.314 | −31.957 | 1.00 | 299.54 | A | C |
| ATOM | 4041 | C | GLY | C | 167 | 9.206 | 18.487 | −31.308 | 1.00 | 329.90 | A | C |
| ATOM | 4042 | O | GLY | C | 167 | 8.587 | 19.098 | −30.442 | 1.00 | 309.33 | A | O |
| ATOM | 4043 | N | LYS | C | 168 | 10.446 | 18.798 | −31.660 | 1.00 | 368.93 | A | N |
| ATOM | 4044 | CA | LYS | C | 168 | 11.237 | 19.736 | −30.818 | 1.00 | 359.08 | A | C |
| ATOM | 4045 | CB | LYS | C | 168 | 12.691 | 19.843 | −31.304 | 1.00 | 302.54 | A | C |
| ATOM | 4046 | CG | LYS | C | 168 | 13.440 | 21.169 | −31.072 | 1.00 | 313.87 | A | C |
| ATOM | 4047 | CD | LYS | C | 168 | 14.696 | 21.268 | −31.961 | 1.00 | 280.44 | A | C |
| ATOM | 4048 | CE | LYS | C | 168 | 14.912 | 22.630 | −32.635 | 1.00 | 295.62 | A | C |
| ATOM | 4049 | NZ | LYS | C | 168 | 15.843 | 22.420 | −33.755 | 1.00 | 321.78 | A | N |
| ATOM | 4050 | C | LYS | C | 168 | 10.585 | 21.098 | −30.493 | 1.00 | 326.62 | A | C |
| ATOM | 4051 | O | LYS | C | 168 | 10.979 | 21.759 | −29.527 | 1.00 | 287.29 | A | O |
| ATOM | 4052 | N | PHE | C | 169 | 9.593 | 21.505 | −31.285 | 1.00 | 344.48 | A | N |
| ATOM | 4053 | CA | PHE | C | 169 | 8.898 | 22.788 | −31.081 | 1.00 | 327.35 | A | C |
| ATOM | 4054 | CB | PHE | C | 169 | 8.853 | 23.668 | −32.391 | 1.00 | 294.91 | A | C |
| ATOM | 4055 | CG | PHE | C | 169 | 9.863 | 24.819 | −32.405 | 1.00 | 311.54 | A | C |
| ATOM | 4056 | CD1 | PHE | C | 169 | 11.229 | 24.566 | −32.559 | 1.00 | 339.62 | A | C |
| ATOM | 4057 | CE1 | PHE | C | 169 | 12.169 | 25.593 | −32.533 | 1.00 | 316.93 | A | C |
| ATOM | 4058 | CZ | PHE | C | 169 | 11.754 | 26.890 | −32.320 | 1.00 | 367.29 | A | C |
| ATOM | 4059 | CE2 | PHE | C | 169 | 10.404 | 27.174 | −32.186 | 1.00 | 380.51 | A | C |
| ATOM | 4060 | CD2 | PHE | C | 169 | 9.463 | 26.148 | −32.232 | 1.00 | 290.26 | A | C |
| ATOM | 4061 | C | PHE | C | 169 | 7.517 | 22.585 | −30.442 | 1.00 | 256.44 | A | C |
| ATOM | 4062 | O | PHE | C | 169 | 6.931 | 23.540 | −29.963 | 1.00 | 238.64 | A | O |
| ATOM | 4063 | N | THR | C | 170 | 7.021 | 21.344 | −30.420 | 1.00 | 247.86 | A | N |
| ATOM | 4064 | CA | THR | C | 170 | 5.746 | 20.983 | −29.729 | 1.00 | 233.90 | A | C |
| ATOM | 4065 | CB | THR | C | 170 | 5.467 | 19.453 | −29.687 | 1.00 | 226.16 | A | C |
| ATOM | 4066 | OG1 | THR | C | 170 | 5.613 | 18.935 | −31.015 | 1.00 | 235.34 | A | O |
| ATOM | 4067 | CG2 | THR | C | 170 | 4.070 | 19.176 | −29.220 | 1.00 | 202.46 | A | C |
| ATOM | 4068 | C | THR | C | 170 | 5.699 | 21.500 | −28.304 | 1.00 | 227.30 | A | C |
| ATOM | 4069 | O | THR | C | 170 | 4.669 | 21.995 | −27.874 | 1.00 | 240.25 | A | O |
| ATOM | 4070 | N | PRO | C | 171 | 6.826 | 21.389 | −27.587 | 1.00 | 239.96 | A | N |
| ATOM | 4071 | CA | PRO | C | 171 | 6.968 | 22.052 | −26.297 | 1.00 | 240.32 | A | C |
| ATOM | 4072 | CB | PRO | C | 171 | 8.328 | 21.546 | −25.784 | 1.00 | 211.40 | A | C |
| ATOM | 4073 | CG | PRO | C | 171 | 9.000 | 20.947 | −26.970 | 1.00 | 237.40 | A | C |
| ATOM | 4074 | CD | PRO | C | 171 | 7.900 | 20.409 | −27.816 | 1.00 | 248.74 | A | C |
| ATOM | 4075 | C | PRO | C | 171 | 6.960 | 23.580 | −26.443 | 1.00 | 230.13 | A | C |
| ATOM | 4076 | O | PRO | C | 171 | 6.280 | 24.264 | −25.673 | 1.00 | 223.63 | A | O |
| ATOM | 4077 | N | TRP | C | 172 | 7.682 | 24.101 | −27.438 | 1.00 | 217.34 | A | N |
| ATOM | 4078 | CA | TRP | C | 172 | 7.779 | 25.551 | −27.629 | 1.00 | 245.35 | A | C |
| ATOM | 4079 | CB | TRP | C | 172 | 8.811 | 25.925 | −28.707 | 1.00 | 244.78 | A | C |
| ATOM | 4080 | CG | TRP | C | 172 | 10.202 | 25.654 | −28.205 | 1.00 | 253.07 | A | C |
| ATOM | 4081 | CD1 | TRP | C | 172 | 11.062 | 24.635 | −28.606 | 1.00 | 270.23 | A | C |
| ATOM | 4082 | NE1 | TRP | C | 172 | 12.233 | 24.676 | −27.887 | 1.00 | 296.67 | A | N |
| ATOM | 4083 | CE2 | TRP | C | 172 | 12.214 | 25.687 | −26.979 | 1.00 | 281.67 | A | C |
| ATOM | 4084 | CD2 | TRP | C | 172 | 10.924 | 26.366 | −27.124 | 1.00 | 268.63 | A | C |
| ATOM | 4085 | CE3 | TRP | C | 172 | 10.637 | 27.445 | −26.295 | 1.00 | 219.84 | A | C |
| ATOM | 4086 | CZ3 | TRP | C | 172 | 11.604 | 27.846 | −25.352 | 1.00 | 287.39 | A | C |
| ATOM | 4087 | CH2 | TRP | C | 172 | 12.837 | 27.194 | −25.240 | 1.00 | 273.00 | A | C |
| ATOM | 4088 | CZ2 | TRP | C | 172 | 13.169 | 26.105 | −26.054 | 1.00 | 299.43 | A | C |
| ATOM | 4089 | C | TRP | C | 172 | 6.440 | 26.150 | −27.907 | 1.00 | 251.28 | A | C |
| ATOM | 4090 | O | TRP | C | 172 | 6.059 | 27.147 | −27.291 | 1.00 | 258.89 | A | O |
| ATOM | 4091 | N | LEU | C | 173 | 5.698 | 25.511 | −28.807 | 1.00 | 247.37 | A | N |
| ATOM | 4092 | CA | LEU | C | 173 | 4.312 | 25.860 | −29.065 | 1.00 | 248.97 | A | C |
| ATOM | 4093 | CB | LEU | C | 173 | 3.642 | 24.771 | −29.900 | 1.00 | 244.02 | A | C |
| ATOM | 4094 | CG | LEU | C | 173 | 2.178 | 24.970 | −30.291 | 1.00 | 232.57 | A | C |
| ATOM | 4095 | CD1 | LEU | C | 173 | 1.966 | 26.320 | −30.939 | 1.00 | 212.49 | A | C |
| ATOM | 4096 | CD2 | LEU | C | 173 | 1.706 | 23.841 | −31.200 | 1.00 | 252.29 | A | C |
| ATOM | 4097 | C | LEU | C | 173 | 3.569 | 26.063 | −27.749 | 1.00 | 212.40 | A | C |
| ATOM | 4098 | O | LEU | C | 173 | 2.876 | 27.061 | −27.578 | 1.00 | 229.84 | A | O |
| ATOM | 4099 | N | ALA | C | 174 | 3.748 | 25.122 | −26.819 | 1.00 | 209.71 | A | N |
| ATOM | 4100 | CA | ALA | C | 174 | 3.089 | 25.173 | −25.522 | 1.00 | 211.49 | A | C |
| ATOM | 4101 | CB | ALA | C | 174 | 3.193 | 23.834 | −24.844 | 1.00 | 217.81 | A | C |
| ATOM | 4102 | C | ALA | C | 174 | 3.649 | 26.260 | −24.618 | 1.00 | 228.67 | A | C |
| ATOM | 4103 | O | ALA | C | 174 | 2.936 | 26.802 | −23.770 | 1.00 | 222.41 | A | O |
| ATOM | 4104 | N | ILE | C | 175 | 4.925 | 26.577 | −24.795 | 1.00 | 222.68 | A | N |
| ATOM | 4105 | CA | ILE | C | 175 | 5.575 | 27.581 | −23.960 | 1.00 | 228.75 | A | C |
| ATOM | 4106 | CB | ILE | C | 175 | 7.108 | 27.475 | −24.003 | 1.00 | 211.77 | A | C |
| ATOM | 4107 | CG1 | ILE | C | 175 | 7.540 | 26.088 | −23.548 | 1.00 | 214.49 | A | C |
| ATOM | 4108 | CD1 | ILE | C | 175 | 8.809 | 25.583 | −24.187 | 1.00 | 212.71 | A | C |
| ATOM | 4109 | CG2 | ILE | C | 175 | 7.715 | 28.525 | −23.079 | 1.00 | 189.66 | A | C |
| ATOM | 4110 | C | ILE | C | 175 | 5.121 | 28.982 | −24.346 | 1.00 | 230.72 | A | C |
| ATOM | 4111 | O | ILE | C | 175 | 4.573 | 29.709 | −23.521 | 1.00 | 252.30 | A | O |
| ATOM | 4112 | N | ILE | C | 176 | 5.350 | 29.353 | −25.598 | 1.00 | 230.85 | A | N |
| ATOM | 4113 | CA | ILE | C | 176 | 4.815 | 30.599 | −26.134 | 1.00 | 261.85 | A | C |
| ATOM | 4114 | CB | ILE | C | 176 | 5.053 | 30.720 | −27.654 | 1.00 | 302.98 | A | C |
| ATOM | 4115 | CG1 | ILE | C | 176 | 4.386 | 29.555 | −28.398 | 1.00 | 253.03 | A | C |
| ATOM | 4116 | CD1 | ILE | C | 176 | 4.661 | 29.492 | −29.890 | 1.00 | 270.40 | A | C |
| ATOM | 4117 | CG2 | ILE | C | 176 | 6.549 | 30.846 | −27.964 | 1.00 | 251.93 | A | C |
| ATOM | 4118 | C | ILE | C | 176 | 3.320 | 30.719 | −25.840 | 1.00 | 248.95 | A | C |
| ATOM | 4119 | O | ILE | C | 176 | 2.867 | 31.741 | −25.314 | 1.00 | 235.78 | A | O |
| ATOM | 4120 | N | GLU | C | 177 | 2.574 | 29.657 | −26.157 | 1.00 | 234.97 | A | N |
| ATOM | 4121 | CA | GLU | C | 177 | 1.131 | 29.631 | −25.947 | 1.00 | 229.48 | A | C |
| ATOM | 4122 | CB | GLU | C | 177 | 0.516 | 28.330 | −26.465 | 1.00 | 221.44 | A | C |
| ATOM | 4123 | CG | GLU | C | 177 | −0.198 | 28.465 | −27.800 | 1.00 | 230.54 | A | C |
| ATOM | 4124 | CD | GLU | C | 177 | −1.026 | 27.244 | −28.144 | 1.00 | 199.36 | A | C |
| ATOM | 4125 | OE1 | GLU | C | 177 | −0.811 | 26.633 | −29.205 | 1.00 | 185.95 | A | O |
| ATOM | 4126 | OE2 | GLU | C | 177 | −1.890 | 26.881 | −27.333 | 1.00 | 223.53 | A | O |
| ATOM | 4127 | C | GLU | C | 177 | 0.772 | 29.857 | −24.486 | 1.00 | 222.52 | A | C |
| ATOM | 4128 | O | GLU | C | 177 | −0.314 | 30.333 | −24.171 | 1.00 | 206.91 | A | O |
| ATOM | 4129 | N | GLY | C | 178 | 1.699 | 29.509 | −23.602 | 1.00 | 233.38 | A | N |
| ATOM | 4130 | CA | GLY | C | 178 | 1.571 | 29.804 | −22.185 | 1.00 | 255.30 | A | C |
| ATOM | 4131 | C | GLY | C | 178 | 1.634 | 31.292 | −21.933 | 1.00 | 221.02 | A | C |
| ATOM | 4132 | O | GLY | C | 178 | 0.671 | 31.867 | −21.438 | 1.00 | 195.56 | A | O |
| ATOM | 4133 | N | ILE | C | 179 | 2.762 | 31.914 | −22.282 | 1.00 | 247.59 | A | N |
| ATOM | 4134 | CA | ILE | C | 179 | 2.929 | 33.359 | −22.095 | 1.00 | 242.53 | A | C |
| ATOM | 4135 | CB | ILE | C | 179 | 4.295 | 33.892 | −22.573 | 1.00 | 247.44 | A | C |
| ATOM | 4136 | CG1 | ILE | C | 179 | 5.458 | 33.241 | −21.842 | 1.00 | 279.89 | A | C |
| ATOM | 4137 | CD1 | ILE | C | 179 | 6.807 | 33.586 | −22.476 | 1.00 | 318.68 | A | C |
| ATOM | 4138 | CG2 | ILE | C | 179 | 4.367 | 35.386 | −22.307 | 1.00 | 244.87 | A | C |
| ATOM | 4139 | C | ILE | C | 179 | 1.853 | 34.136 | −22.831 | 1.00 | 207.77 | A | C |
| ATOM | 4140 | O | ILE | C | 179 | 1.257 | 35.070 | −22.288 | 1.00 | 213.09 | A | O |
| ATOM | 4141 | N | LEU | C | 180 | 1.616 | 33.737 | −24.073 | 1.00 | 218.71 | A | N |
| ATOM | 4142 | CA | LEU | C | 180 | 0.698 | 34.467 | −24.943 | 1.00 | 239.01 | A | C |
| ATOM | 4143 | CB | LEU | C | 180 | 1.002 | 34.165 | −26.413 | 1.00 | 204.72 | A | C |
| ATOM | 4144 | CG | LEU | C | 180 | 1.728 | 35.138 | −27.326 | 1.00 | 220.83 | A | C |
| ATOM | 4145 | CD1 | LEU | C | 180 | 3.173 | 35.330 | −26.859 | 1.00 | 239.37 | A | C |
| ATOM | 4146 | CD2 | LEU | C | 180 | 1.663 | 34.561 | −28.741 | 1.00 | 251.05 | A | C |
| ATOM | 4147 | C | LEU | C | 180 | −0.761 | 34.146 | −24.701 | 1.00 | 231.54 | A | C |
| ATOM | 4148 | O | LEU | C | 180 | −1.526 | 34.986 | −24.249 | 1.00 | 210.05 | A | O |
| ATOM | 4149 | N | THR | C | 181 | −1.135 | 32.912 | −25.004 | 1.00 | 227.46 | A | N |
| ATOM | 4150 | CA | THR | C | 181 | −2.532 | 32.579 | −25.257 | 1.00 | 232.55 | A | C |
| ATOM | 4151 | CB | THR | C | 181 | −2.661 | 31.273 | −26.073 | 1.00 | 238.59 | A | C |
| ATOM | 4152 | OG1 | THR | C | 181 | −1.672 | 31.246 | −27.105 | 1.00 | 288.11 | A | O |
| ATOM | 4153 | CG2 | THR | C | 181 | −4.005 | 31.197 | −26.735 | 1.00 | 201.32 | A | C |
| ATOM | 4154 | C | THR | C | 181 | −3.353 | 32.476 | −23.986 | 1.00 | 196.62 | A | C |
| ATOM | 4155 | O | THR | C | 181 | −4.495 | 32.900 | −23.945 | 1.00 | 234.94 | A | O |
| ATOM | 4156 | N | ALA | C | 182 | −2.759 | 31.933 | −22.947 | 1.00 | 228.88 | A | N |
| ATOM | 4157 | CA | ALA | C | 182 | −3.462 | 31.754 | −21.691 | 1.00 | 238.22 | A | C |
| ATOM | 4158 | CB | ALA | C | 182 | −3.139 | 30.399 | −21.094 | 1.00 | 178.58 | A | C |
| ATOM | 4159 | C | ALA | C | 182 | −3.124 | 32.846 | −20.701 | 1.00 | 216.13 | A | C |
| ATOM | 4160 | O | ALA | C | 182 | −4.011 | 33.369 | −20.036 | 1.00 | 221.44 | A | O |
| ATOM | 4161 | N | TRP | C | 183 | −1.844 | 33.200 | −20.622 | 1.00 | 208.12 | A | N |
| ATOM | 4162 | CA | TRP | C | 183 | −1.316 | 33.978 | −19.509 | 1.00 | 198.34 | A | C |
| ATOM | 4163 | CB | TRP | C | 183 | 0.200 | 33.966 | −19.544 | 1.00 | 209.16 | A | C |
| ATOM | 4164 | CG | TRP | C | 183 | 0.864 | 34.639 | −18.365 | 1.00 | 196.15 | A | C |
| ATOM | 4165 | CD1 | TRP | C | 183 | 0.290 | 35.498 | −17.422 | 1.00 | 188.19 | A | C |
| ATOM | 4166 | NE1 | TRP | C | 183 | 1.219 | 35.926 | −16.514 | 1.00 | 209.86 | A | N |
| ATOM | 4167 | CE2 | TRP | C | 183 | 2.439 | 35.412 | −16.806 | 1.00 | 217.53 | A | C |
| ATOM | 4168 | CD2 | TRP | C | 183 | 2.282 | 34.574 | −18.002 | 1.00 | 207.33 | A | C |
| ATOM | 4169 | CE3 | TRP | C | 183 | 3.395 | 33.911 | −18.516 | 1.00 | 217.79 | A | C |
| ATOM | 4170 | CZ3 | TRP | C | 183 | 4.627 | 34.076 | −17.873 | 1.00 | 197.71 | A | C |
| ATOM | 4171 | CH2 | TRP | C | 183 | 4.759 | 34.882 | −16.732 | 1.00 | 201.28 | A | C |
| ATOM | 4172 | CZ2 | TRP | C | 183 | 3.670 | 35.566 | −16.178 | 1.00 | 202.60 | A | C |
| ATOM | 4173 | C | TRP | C | 183 | −1.800 | 35.393 | −19.504 | 1.00 | 214.77 | A | C |
| ATOM | 4174 | O | TRP | C | 183 | −2.461 | 35.815 | −18.564 | 1.00 | 211.15 | A | O |
| ATOM | 4175 | N | ILE | C | 184 | −1.442 | 36.143 | −20.544 | 1.00 | 213.99 | A | N |
| ATOM | 4176 | CA | ILE | C | 184 | −1.767 | 37.554 | −20.640 | 1.00 | 235.76 | A | C |
| ATOM | 4177 | CB | ILE | C | 184 | −1.012 | 38.204 | −21.815 | 1.00 | 272.61 | A | C |
| ATOM | 4178 | CG1 | ILE | C | 184 | 0.468 | 38.345 | −21.422 | 1.00 | 214.63 | A | C |
| ATOM | 4179 | CD1 | ILE | C | 184 | 1.420 | 38.255 | −22.580 | 1.00 | 200.51 | A | C |
| ATOM | 4180 | CG2 | ILE | C | 184 | −1.583 | 39.557 | −22.148 | 1.00 | 205.00 | A | C |
| ATOM | 4181 | C | ILE | C | 184 | −3.277 | 37.775 | −20.686 | 1.00 | 240.27 | A | C |
| ATOM | 4182 | O | ILE | C | 184 | −3.789 | 38.583 | −19.920 | 1.00 | 227.02 | A | O |
| ATOM | 4183 | N | PRO | C | 185 | −3.994 | 37.042 | −21.560 | 1.00 | 231.20 | A | N |
| ATOM | 4184 | CA | PRO | C | 185 | −5.448 | 37.177 | −21.687 | 1.00 | 240.04 | A | C |
| ATOM | 4185 | CB | PRO | C | 185 | −5.821 | 36.001 | −22.588 | 1.00 | 238.01 | A | C |
| ATOM | 4186 | CG | PRO | C | 185 | −4.637 | 35.866 | −23.469 | 1.00 | 217.27 | A | C |
| ATOM | 4187 | CD | PRO | C | 185 | −3.472 | 36.071 | −22.535 | 1.00 | 222.29 | A | C |
| ATOM | 4188 | C | PRO | C | 185 | −6.164 | 37.055 | −20.355 | 1.00 | 205.89 | A | C |
| ATOM | 4189 | O | PRO | C | 185 | −7.219 | 37.642 | −20.167 | 1.00 | 226.31 | A | O |
| ATOM | 4190 | N | ALA | C | 186 | −5.586 | 36.289 | −19.441 | 1.00 | 177.33 | A | N |
| ATOM | 4191 | CA | ALA | C | 186 | −6.124 | 36.156 | −18.096 | 1.00 | 203.45 | A | C |
| ATOM | 4192 | CB | ALA | C | 186 | −5.586 | 34.896 | −17.434 | 1.00 | 201.93 | A | C |
| ATOM | 4193 | C | ALA | C | 186 | −5.781 | 37.391 | −17.269 | 1.00 | 239.59 | A | C |
| ATOM | 4194 | O | ALA | C | 186 | −6.668 | 38.038 | −16.708 | 1.00 | 209.11 | A | O |
| ATOM | 4195 | N | TRP | C | 187 | −4.491 | 37.719 | −17.217 | 1.00 | 233.05 | A | N |
| ATOM | 4196 | CA | TRP | C | 187 | −4.021 | 38.909 | −16.540 | 1.00 | 189.07 | A | C |
| ATOM | 4197 | CB | TRP | C | 187 | −2.498 | 38.956 | −16.532 | 1.00 | 209.18 | A | C |
| ATOM | 4198 | CG | TRP | C | 187 | −1.979 | 40.116 | −15.746 | 1.00 | 227.50 | A | C |
| ATOM | 4199 | CD1 | TRP | C | 187 | −1.589 | 41.352 | −16.241 | 1.00 | 273.45 | A | C |
| ATOM | 4200 | NE1 | TRP | C | 187 | −1.198 | 42.189 | −15.227 | 1.00 | 273.21 | A | N |
| ATOM | 4201 | CE2 | TRP | C | 187 | −1.320 | 41.575 | −14.031 | 1.00 | 242.61 | A | C |
| ATOM | 4202 | CD2 | TRP | C | 187 | −1.823 | 40.225 | −14.291 | 1.00 | 204.97 | A | C |
| ATOM | 4203 | CE3 | TRP | C | 187 | −2.047 | 39.388 | −13.227 | 1.00 | 215.63 | A | C |
| ATOM | 4204 | CZ3 | TRP | C | 187 | −1.770 | 39.853 | −11.934 | 1.00 | 245.98 | A | C |
| ATOM | 4205 | CH2 | TRP | C | 187 | −1.280 | 41.139 | −11.701 | 1.00 | 222.45 | A | C |
| ATOM | 4206 | CZ2 | TRP | C | 187 | −1.051 | 42.027 | −12.745 | 1.00 | 226.50 | A | C |
| ATOM | 4207 | C | TRP | C | 187 | −4.634 | 40.133 | −17.181 | 1.00 | 216.81 | A | C |
| ATOM | 4208 | O | TRP | C | 187 | −4.757 | 41.182 | −16.546 | 1.00 | 236.40 | A | O |
| ATOM | 4209 | N | LEU | C | 188 | −5.053 | 39.996 | −18.442 | 1.00 | 204.21 | A | N |
| ATOM | 4210 | CA | LEU | C | 188 | −5.739 | 41.049 | −19.178 | 1.00 | 195.01 | A | C |
| ATOM | 4211 | CB | LEU | C | 188 | −5.257 | 41.065 | −20.635 | 1.00 | 201.39 | A | C |
| ATOM | 4212 | CG | LEU | C | 188 | −5.919 | 41.905 | −21.726 | 1.00 | 203.75 | A | C |
| ATOM | 4213 | CD1 | LEU | C | 188 | −4.882 | 42.346 | −22.741 | 1.00 | 227.26 | A | C |
| ATOM | 4214 | CD2 | LEU | C | 188 | −7.039 | 41.133 | −22.390 | 1.00 | 203.00 | A | C |
| ATOM | 4215 | C | LEU | C | 188 | −7.256 | 40.871 | −19.122 | 1.00 | 215.67 | A | C |
| ATOM | 4216 | O | LEU | C | 188 | −8.016 | 41.744 | −19.580 | 1.00 | 212.28 | A | O |
| ATOM | 4217 | N | LEU | C | 189 | −7.692 | 39.735 | −18.585 | 1.00 | 213.87 | A | N |
| ATOM | 4218 | CA | LEU | C | 189 | −9.107 | 39.524 | −18.334 | 1.00 | 226.30 | A | C |
| ATOM | 4219 | CB | LEU | C | 189 | −9.415 | 38.039 | −18.262 | 1.00 | 203.07 | A | C |
| ATOM | 4220 | CG | LEU | C | 189 | −10.708 | 37.595 | −18.938 | 1.00 | 229.15 | A | C |
| ATOM | 4221 | CD1 | LEU | C | 189 | −10.754 | 38.055 | −20.390 | 1.00 | 242.00 | A | C |
| ATOM | 4222 | CD2 | LEU | C | 189 | −10.832 | 36.083 | −18.862 | 1.00 | 228.15 | A | C |
| ATOM | 4223 | C | LEU | C | 189 | −9.497 | 40.186 | −17.028 | 1.00 | 230.12 | A | C |
| ATOM | 4224 | O | LEU | C | 189 | −10.666 | 40.449 | −16.779 | 1.00 | 235.33 | A | O |
| ATOM | 4225 | N | PHE | C | 190 | −8.507 | 40.429 | −16.180 | 1.00 | 218.20 | A | N |
| ATOM | 4226 | CA | PHE | C | 190 | −8.733 | 41.160 | −14.951 | 1.00 | 203.89 | A | C |
| ATOM | 4227 | CB | PHE | C | 190 | −7.540 | 41.010 | −14.004 | 1.00 | 215.59 | A | C |
| ATOM | 4228 | CG | PHE | C | 190 | −7.350 | 39.627 | −13.466 | 1.00 | 202.01 | A | C |
| ATOM | 4229 | CD1 | PHE | C | 190 | −8.400 | 38.943 | −12.878 | 1.00 | 216.27 | A | C |
| ATOM | 4230 | CE1 | PHE | C | 190 | −8.222 | 37.661 | −12.379 | 1.00 | 224.68 | A | C |
| ATOM | 4231 | CZ | PHE | C | 190 | −6.977 | 37.058 | −12.456 | 1.00 | 232.81 | A | C |
| ATOM | 4232 | CE2 | PHE | C | 190 | −5.920 | 37.737 | −13.032 | 1.00 | 220.52 | A | C |
| ATOM | 4233 | CD2 | PHE | C | 190 | −6.112 | 39.018 | −13.526 | 1.00 | 194.19 | A | C |
| ATOM | 4234 | C | PHE | C | 190 | −8.970 | 42.639 | −15.240 | 1.00 | 232.44 | A | C |
| ATOM | 4235 | O | PHE | C | 190 | −9.674 | 43.316 | −14.493 | 1.00 | 254.23 | A | O |
| ATOM | 4236 | N | ILE | C | 191 | −8.417 | 43.120 | −16.352 | 1.00 | 217.28 | A | N |
| ATOM | 4237 | CA | ILE | C | 191 | −8.324 | 44.548 | −16.633 | 1.00 | 238.42 | A | C |
| ATOM | 4238 | CB | ILE | C | 191 | −6.865 | 44.926 | −16.993 | 1.00 | 240.74 | A | C |
| ATOM | 4239 | CG1 | ILE | C | 191 | −5.992 | 44.875 | −15.728 | 1.00 | 274.66 | A | C |
| ATOM | 4240 | CD1 | ILE | C | 191 | −4.515 | 45.134 | −15.944 | 1.00 | 304.89 | A | C |
| ATOM | 4241 | CG2 | ILE | C | 191 | −6.787 | 46.299 | −17.637 | 1.00 | 195.94 | A | C |
| ATOM | 4242 | C | ILE | C | 191 | −9.313 | 45.060 | −17.691 | 1.00 | 234.63 | A | C |
| ATOM | 4243 | O | ILE | C | 191 | −10.077 | 45.988 | −17.423 | 1.00 | 233.04 | A | O |
| ATOM | 4244 | N | GLN | C | 192 | −9.279 | 44.476 | −18.888 | 1.00 | 238.12 | A | N |
| ATOM | 4245 | CA | GLN | C | 192 | −10.189 | 44.858 | −19.969 | 1.00 | 231.00 | A | C |
| ATOM | 4246 | CB | GLN | C | 192 | −9.481 | 44.887 | −21.331 | 1.00 | 235.19 | A | C |
| ATOM | 4247 | CG | GLN | C | 192 | −8.246 | 45.785 | −21.413 | 1.00 | 223.13 | A | C |
| ATOM | 4248 | CD | GLN | C | 192 | −8.558 | 47.278 | −21.512 | 1.00 | 217.15 | A | C |
| ATOM | 4249 | OE1 | GLN | C | 192 | −9.631 | 47.669 | −21.954 | 1.00 | 223.22 | A | O |
| ATOM | 4250 | NE2 | GLN | C | 192 | −7.601 | 48.116 | −21.118 | 1.00 | 215.68 | A | N |
| ATOM | 4251 | C | GLN | C | 192 | −11.343 | 43.883 | −19.992 | 1.00 | 207.92 | A | C |
| ATOM | 4252 | O | GLN | C | 192 | −12.340 | 44.110 | −20.662 | 1.00 | 246.23 | A | O |
| ATOM | 4253 | N | HIS | C | 193 | −11.185 | 42.779 | −19.262 | 1.00 | 239.20 | A | N |
| ATOM | 4254 | CA | HIS | C | 193 | −12.315 | 41.921 | −18.875 | 1.00 | 284.08 | A | C |
| ATOM | 4255 | CB | HIS | C | 193 | −13.491 | 42.768 | −18.381 | 1.00 | 275.59 | A | C |
| ATOM | 4256 | CG | HIS | C | 193 | −13.121 | 43.719 | −17.265 | 1.00 | 251.79 | A | C |
| ATOM | 4257 | ND1 | HIS | C | 193 | −14.014 | 44.170 | −16.358 | 1.00 | 258.71 | A | N |
| ATOM | 4258 | CE1 | HIS | C | 193 | −13.391 | 44.992 | −15.490 | 1.00 | 237.40 | A | C |
| ATOM | 4259 | NE2 | HIS | C | 193 | −12.098 | 45.064 | −15.840 | 1.00 | 246.84 | A | N |
| ATOM | 4260 | CD2 | HIS | C | 193 | −11.898 | 44.286 | −16.923 | 1.00 | 245.78 | A | C |
| ATOM | 4261 | C | HIS | C | 193 | −12.756 | 40.934 | −19.912 | 1.00 | 272.22 | A | C |
| ATOM | 4262 | O | HIS | C | 193 | −12.891 | 39.752 | −19.600 | 1.00 | 285.68 | A | O |
| ATOM | 4263 | N | TRP | C | 194 | −13.013 | 41.388 | −21.137 | 1.00 | 282.05 | A | N |
| ATOM | 4264 | CA | TRP | C | 194 | −13.191 | 40.441 | −22.247 | 1.00 | 256.27 | A | C |
| ATOM | 4265 | CB | TRP | C | 194 | −14.617 | 40.441 | −22.787 | 1.00 | 243.21 | A | C |
| ATOM | 4266 | CG | TRP | C | 194 | −15.536 | 39.549 | −22.000 | 1.00 | 254.18 | A | C |
| ATOM | 4267 | CD1 | TRP | C | 194 | −16.401 | 39.923 | −20.979 | 1.00 | 275.43 | A | C |
| ATOM | 4268 | NE1 | TRP | C | 194 | −17.077 | 38.835 | −20.486 | 1.00 | 326.73 | A | N |
| ATOM | 4269 | CE2 | TRP | C | 194 | −16.712 | 37.701 | −21.129 | 1.00 | 317.80 | A | C |
| ATOM | 4270 | CD2 | TRP | C | 194 | −15.706 | 38.085 | −22.127 | 1.00 | 263.00 | A | C |
| ATOM | 4271 | CE3 | TRP | C | 194 | −15.160 | 37.099 | −22.935 | 1.00 | 271.15 | A | C |
| ATOM | 4272 | CZ3 | TRP | C | 194 | −15.604 | 35.770 | −22.765 | 1.00 | 255.02 | A | C |
| ATOM | 4273 | CH2 | TRP | C | 194 | −16.558 | 35.418 | −21.801 | 1.00 | 254.22 | A | C |
| ATOM | 4274 | CZ2 | TRP | C | 194 | −17.132 | 36.376 | −20.966 | 1.00 | 310.12 | A | C |
| ATOM | 4275 | C | TRP | C | 194 | −12.195 | 40.668 | −23.343 | 1.00 | 265.90 | A | C |
| ATOM | 4276 | O | TRP | C | 194 | −11.735 | 41.797 | −23.545 | 1.00 | 238.46 | A | O |
| ATOM | 4277 | N | VAL | C | 195 | −11.823 | 39.586 | −24.034 | 1.00 | 277.16 | A | N |
| ATOM | 4278 | CA | VAL | C | 195 | −10.975 | 39.674 | −25.223 | 1.00 | 271.79 | A | C |
| ATOM | 4279 | CB | VAL | C | 195 | −11.672 | 40.530 | −26.317 | 1.00 | 266.51 | A | C |
| ATOM | 4280 | CG1 | VAL | C | 195 | −10.882 | 40.566 | −27.617 | 1.00 | 254.12 | A | C |
| ATOM | 4281 | CG2 | VAL | C | 195 | −13.078 | 40.027 | −26.560 | 1.00 | 243.93 | A | C |
| ATOM | 4282 | C | VAL | C | 195 | −9.615 | 40.299 | −24.874 | 1.00 | 267.90 | A | C |
| ATOM | 4283 | O | VAL | C | 195 | −9.368 | 40.761 | −23.756 | 1.00 | 230.31 | A | O |
| ATOM | 4284 | OXT | VAL | C | 195 | −8.711 | 40.348 | −25.709 | 1.00 | 255.92 | | O |
| ATOM | 4285 | O8 | XP4 | D | 301 | 3.317 | 9.768 | −8.624 | 1.00 | 158.87 | A_3 | O |
| ATOM | 4286 | C18 | XP4 | D | 301 | 3.134 | 9.698 | −7.404 | 1.00 | 170.48 | A_3 | C |
| ATOM | 4287 | C19 | XP4 | D | 301 | 2.613 | 10.927 | −6.629 | 1.00 | 131.42 | A_3 | C |
| ATOM | 4288 | C20 | XP4 | D | 301 | 2.035 | 12.093 | −7.447 | 1.00 | 89.02 | A_3 | C |
| ATOM | 4289 | C21 | XP4 | D | 301 | 1.397 | 13.179 | −6.583 | 1.00 | 109.73 | A_3 | C |
| ATOM | 4290 | C22 | XP4 | D | 301 | 0.049 | 13.586 | −7.170 | 1.00 | 129.38 | A_3 | C |
| ATOM | 4291 | C23 | XP4 | D | 301 | −0.506 | 14.903 | −6.637 | 1.00 | 70.50 | A_3 | C |
| ATOM | 4292 | C24 | XP4 | D | 301 | −2.060 | 15.013 | −6.622 | 1.00 | 129.01 | A_3 | C |
| ATOM | 4293 | C25 | XP4 | D | 301 | −2.609 | 16.416 | −6.219 | 1.00 | 108.91 | A_3 | C |
| ATOM | 4294 | C26 | XP4 | D | 301 | −3.951 | 16.887 | −6.885 | 1.00 | 118.98 | A_3 | C |
| ATOM | 4295 | C27 | XP4 | D | 301 | −4.657 | 18.093 | −6.207 | 1.00 | 132.35 | A_3 | C |
| ATOM | 4296 | C28 | XP4 | D | 301 | −5.105 | 19.269 | −7.081 | 1.00 | 92.62 | A_3 | C |
| ATOM | 4297 | C29 | XP4 | D | 301 | −5.275 | 20.492 | −6.197 | 1.00 | 117.51 | A_3 | C |
| ATOM | 4298 | C30 | XP4 | D | 301 | −5.877 | 21.632 | −7.045 | 1.00 | 135.50 | A_3 | C |
| ATOM | 4299 | C31 | XP4 | D | 301 | −7.043 | 22.425 | −6.429 | 1.00 | 114.72 | A_3 | C |
| ATOM | 4300 | O7 | XP4 | D | 301 | 3.444 | 8.393 | −6.698 | 1.00 | 151.06 | A_3 | O |
| ATOM | 4301 | C2 | XP4 | D | 301 | 2.504 | 7.286 | −6.854 | 1.00 | 134.14 | A_3 | C |
| ATOM | 4302 | C1 | XP4 | D | 301 | 3.000 | 5.854 | −6.556 | 1.00 | 176.45 | A_3 | C |
| ATOM | 4303 | O4 | XP4 | D | 301 | 3.069 | 4.875 | −7.612 | 1.00 | 287.22 | A_3 | O |
| ATOM | 4304 | P1 | XP4 | D | 301 | 2.171 | 3.716 | −8.455 | 1.00 | 229.09 | A_3 | P |
| ATOM | 4305 | O3 | XP4 | D | 301 | 0.898 | 3.503 | −7.643 | 1.00 | 298.01 | A_3 | O |
| ATOM | 4306 | O2 | XP4 | D | 301 | 1.923 | 3.898 | −10.009 | 1.00 | 218.59 | A_3 | O |
| ATOM | 4307 | O1 | XP4 | D | 301 | 3.163 | 2.540 | −8.356 | 1.00 | 254.60 | A_3 | O |
| ATOM | 4308 | C3 | XP4 | D | 301 | 1.372 | 7.301 | −5.827 | 1.00 | 158.00 | A_3 | C |
| ATOM | 4309 | O5 | XP4 | D | 301 | 0.827 | 8.613 | −5.518 | 1.00 | 170.11 | A_3 | O |
| ATOM | 4310 | C4 | XP4 | D | 301 | −0.542 | 8.999 | −5.791 | 1.00 | 153.72 | A_3 | C |
| ATOM | 4311 | O6 | XP4 | D | 301 | −1.383 | 8.593 | −5.079 | 1.00 | 169.21 | A_3 | O |
| ATOM | 4312 | C5 | XP4 | D | 301 | −1.310 | 9.708 | −6.817 | 1.00 | 150.59 | A_3 | C |
| ATOM | 4313 | C6 | XP4 | D | 301 | −2.806 | 9.892 | −6.502 | 1.00 | 147.71 | A_3 | C |
| ATOM | 4314 | C7 | XP4 | D | 301 | −3.487 | 10.770 | −7.506 | 1.00 | 102.39 | A_3 | C |
| ATOM | 4315 | C8 | XP4 | D | 301 | −4.829 | 10.138 | −7.843 | 1.00 | 99.81 | A_3 | C |
| ATOM | 4316 | C9 | XP4 | D | 301 | −5.543 | 11.039 | −8.840 | 1.00 | 104.12 | A_3 | C |
| ATOM | 4317 | C10 | XP4 | D | 301 | −7.043 | 10.911 | −8.680 | 1.00 | 92.75 | A_3 | C |
| ATOM | 4318 | C11 | XP4 | D | 301 | −7.693 | 12.119 | −9.321 | 1.00 | 118.76 | A_3 | C |
| ATOM | 4319 | C12 | XP4 | D | 301 | −9.141 | 12.293 | −8.873 | 1.00 | 114.49 | A_3 | C |
| ATOM | 4320 | C13 | XP4 | D | 301 | −9.688 | 13.625 | −9.407 | 1.00 | 171.77 | A_3 | C |
| ATOM | 4321 | C14 | XP4 | D | 301 | −10.215 | 14.594 | −8.310 | 1.00 | 177.72 | A_3 | C |
| ATOM | 4322 | C15 | XP4 | D | 301 | −9.695 | 16.052 | −8.391 | 1.00 | 187.85 | A_3 | C |
| ATOM | 4323 | C16 | XP4 | D | 301 | −10.693 | 17.102 | −7.845 | 1.00 | 131.15 | A_3 | C |
| ATOM | 4324 | C17 | XP4 | D | 301 | −9.958 | 18.280 | −7.224 | 1.00 | 179.33 | A_3 | C |
| ATOM | 4325 | O8 | XP4 | E | 301 | 3.789 | 12.491 | 4.004 | 1.00 | 163.65 | A_1 | O |
| ATOM | 4326 | C18 | XP4 | E | 301 | 3.020 | 11.688 | 4.485 | 1.00 | 164.12 | A_1 | C |
| ATOM | 4327 | C19 | XP4 | E | 301 | 1.846 | 12.227 | 5.291 | 1.00 | 130.83 | A_1 | C |
| ATOM | 4328 | C20 | XP4 | E | 301 | 1.652 | 13.723 | 5.068 | 1.00 | 94.45 | A_1 | C |
| ATOM | 4329 | C21 | XP4 | E | 301 | 0.353 | 14.203 | 5.697 | 1.00 | 89.50 | A_1 | C |
| ATOM | 4330 | C22 | XP4 | E | 301 | −0.460 | 15.114 | 4.765 | 1.00 | 104.71 | A_1 | C |
| ATOM | 4331 | C23 | XP4 | E | 301 | −1.598 | 15.801 | 5.525 | 1.00 | 105.97 | A_1 | C |
| ATOM | 4332 | C24 | XP4 | E | 301 | −2.639 | 16.376 | 4.560 | 1.00 | 126.51 | A_1 | C |
| ATOM | 4333 | C25 | XP4 | E | 301 | −3.664 | 17.279 | 5.251 | 1.00 | 121.29 | A_1 | C |
| ATOM | 4334 | C26 | XP4 | E | 301 | −4.279 | 18.398 | 4.385 | 1.00 | 154.45 | A_1 | C |
| ATOM | 4335 | C27 | XP4 | E | 301 | −5.591 | 18.977 | 4.962 | 1.00 | 171.69 | A_1 | C |
| ATOM | 4336 | C28 | XP4 | E | 301 | −5.772 | 20.511 | 5.025 | 1.00 | 122.73 | A_1 | C |
| ATOM | 4337 | C29 | XP4 | E | 301 | −6.821 | 20.973 | 6.067 | 1.00 | 130.07 | A_1 | C |
| ATOM | 4338 | C30 | XP4 | E | 301 | −6.980 | 22.504 | 6.026 | 1.00 | 130.06 | A_1 | C |
| ATOM | 4339 | C31 | XP4 | E | 301 | −8.406 | 23.053 | 6.065 | 1.00 | 94.13 | A_1 | C |
| ATOM | 4340 | O7 | XP4 | E | 301 | 3.353 | 10.254 | 4.322 | 1.00 | 115.92 | A_1 | O |
| ATOM | 4341 | C2 | XP4 | E | 301 | 3.107 | 9.749 | 3.008 | 1.00 | 134.48 | A_1 | C |
| ATOM | 4342 | C1 | XP4 | E | 301 | 4.058 | 8.545 | 2.829 | 1.00 | 148.57 | A_1 | C |
| ATOM | 4343 | O4 | XP4 | E | 301 | 4.033 | 7.739 | 1.644 | 1.00 | 133.43 | A_1 | O |
| ATOM | 4344 | C3 | XP4 | E | 301 | 1.650 | 9.314 | 2.918 | 1.00 | 170.15 | A_1 | C |
| ATOM | 4345 | O5 | XP4 | E | 301 | 0.738 | 10.319 | 3.417 | 1.00 | 172.46 | A_1 | O |
| ATOM | 4346 | C4 | XP4 | E | 301 | 0.243 | 11.303 | 2.480 | 1.00 | 157.34 | A_1 | C |
| ATOM | 4347 | O6 | XP4 | E | 301 | 0.445 | 11.027 | 1.359 | 1.00 | 173.32 | A_1 | O |
| ATOM | 4348 | C5 | XP4 | E | 301 | −0.556 | 12.575 | 2.621 | 1.00 | 107.01 | A_1 | C |
| ATOM | 4349 | C6 | XP4 | E | 301 | −1.404 | 12.845 | 1.339 | 1.00 | 126.62 | A_1 | C |
| ATOM | 4350 | C7 | XP4 | E | 301 | −1.603 | 14.312 | 0.936 | 1.00 | 115.52 | A_1 | C |
| ATOM | 4351 | C8 | XP4 | E | 301 | −2.170 | 14.362 | −0.477 | 1.00 | 189.32 | A_1 | C |
| ATOM | 4352 | C9 | XP4 | E | 301 | −2.279 | 15.819 | −0.933 | 1.00 | 147.94 | A_1 | C |
| ATOM | 4353 | C10 | XP4 | E | 301 | −3.381 | 16.093 | −1.966 | 1.00 | 153.33 | A_1 | C |
| ATOM | 4354 | C11 | XP4 | E | 301 | −3.900 | 17.536 | −1.818 | 1.00 | 196.94 | A_1 | C |
| ATOM | 4355 | C12 | XP4 | E | 301 | −5.152 | 17.704 | −2.655 | 1.00 | 137.31 | A_1 | C |
| ATOM | 4356 | C13 | XP4 | E | 301 | −5.588 | 19.155 | −2.509 | 1.00 | 170.29 | A_1 | C |
| ATOM | 4357 | C14 | XP4 | E | 301 | −6.878 | 19.218 | −1.699 | 1.00 | 210.94 | A_1 | C |
| ATOM | 4358 | C15 | XP4 | E | 301 | −6.867 | 20.223 | −0.544 | 1.00 | 182.64 | A_1 | C |
| ATOM | 4359 | C16 | XP4 | E | 301 | −8.253 | 20.872 | −0.404 | 1.00 | 184.62 | A_1 | C |
| ATOM | 4360 | C17 | XP4 | E | 301 | −8.630 | 21.017 | 1.053 | 1.00 | 166.37 | A_1 | C |
| ATOM | 4361 | O8 | XP4 | F | 301 | −2.606 | 5.234 | 13.045 | 1.00 | 135.70 | A_2 | O |
| ATOM | 4362 | C18 | XP4 | F | 301 | −3.248 | 4.568 | 12.238 | 1.00 | 168.30 | A_2 | C |
| ATOM | 4363 | C19 | XP4 | F | 301 | −4.745 | 4.901 | 12.077 | 1.00 | 138.62 | A_2 | C |
| ATOM | 4364 | C20 | XP4 | F | 301 | −5.141 | 6.337 | 12.473 | 1.00 | 80.58 | A_2 | C |
| ATOM | 4365 | C21 | XP4 | F | 301 | −6.627 | 6.661 | 12.225 | 1.00 | 107.55 | A_2 | C |
| ATOM | 4366 | C22 | XP4 | F | 301 | −6.858 | 8.150 | 11.891 | 1.00 | 120.18 | A_2 | C |
| ATOM | 4367 | C23 | XP4 | F | 301 | −8.313 | 8.522 | 11.617 | 1.00 | 88.94 | A_2 | C |
| ATOM | 4368 | C24 | XP4 | F | 301 | −8.465 | 9.797 | 10.790 | 1.00 | 109.90 | A_2 | C |
| ATOM | 4369 | C25 | XP4 | F | 301 | −9.869 | 10.395 | 10.977 | 1.00 | 151.96 | A_2 | C |
| ATOM | 4370 | C26 | XP4 | F | 301 | −10.004 | 11.918 | 10.793 | 1.00 | 153.59 | A_2 | C |
| ATOM | 4371 | C27 | XP4 | F | 301 | −11.456 | 12.375 | 10.522 | 1.00 | 113.89 | A_2 | C |
| ATOM | 4372 | C28 | XP4 | F | 301 | −11.989 | 13.597 | 11.309 | 1.00 | 152.90 | A_2 | C |
| ATOM | 4373 | C29 | XP4 | F | 301 | −13.543 | 13.713 | 11.372 | 1.00 | 146.20 | A_2 | C |
| ATOM | 4374 | C30 | XP4 | F | 301 | −14.046 | 14.961 | 12.135 | 1.00 | 143.41 | A_2 | C |
| ATOM | 4375 | C31 | XP4 | F | 301 | −15.247 | 15.679 | 11.515 | 1.00 | 123.23 | A_2 | C |
| ATOM | 4376 | O7 | XP4 | F | 301 | −2.542 | 3.480 | 11.475 | 1.00 | 125.06 | A_2 | O |
| ATOM | 4377 | C2 | XP4 | F | 301 | −1.769 | 4.016 | 10.383 | 1.00 | 138.88 | A_2 | C |
| ATOM | 4378 | C1 | XP4 | F | 301 | −0.389 | 3.383 | 10.253 | 1.00 | 105.44 | A_2 | C |
| ATOM | 4379 | O4 | XP4 | F | 301 | −0.135 | 2.475 | 9.232 | 1.00 | 220.19 | A_2 | O |
| ATOM | 4380 | P1 | XP4 | F | 301 | −0.311 | 2.677 | 7.638 | 1.00 | 245.13 | A_2 | P |
| ATOM | 4381 | O3 | XP4 | F | 301 | 0.464 | 1.481 | 7.126 | 1.00 | 280.78 | A_2 | O |
| ATOM | 4382 | O2 | XP4 | F | 301 | −1.791 | 2.612 | 7.284 | 1.00 | 235.38 | A_2 | O |
| ATOM | 4383 | O1 | XP4 | F | 301 | 0.298 | 4.050 | 7.404 | 1.00 | 181.79 | A_2 | O |
| ATOM | 4384 | C3 | XP4 | F | 301 | −2.611 | 4.054 | 9.082 | 1.00 | 175.77 | A_2 | C |
| ATOM | 4385 | O5 | XP4 | F | 301 | −3.828 | 4.853 | 9.181 | 1.00 | 164.88 | A_2 | O |
| ATOM | 4386 | C4 | XP4 | F | 301 | −3.944 | 6.299 | 8.891 | 1.00 | 145.13 | A_2 | C |
| ATOM | 4387 | O6 | XP4 | F | 301 | −3.091 | 7.009 | 9.208 | 1.00 | 101.95 | A_2 | O |
| ATOM | 4388 | C5 | XP4 | F | 301 | −5.035 | 7.151 | 8.386 | 1.00 | 162.58 | A_2 | C |
| ATOM | 4389 | C6 | XP4 | F | 301 | −4.569 | 8.439 | 7.675 | 1.00 | 145.03 | A_2 | C |
| ATOM | 4390 | C7 | XP4 | F | 301 | −4.710 | 9.790 | 8.347 | 1.00 | 152.34 | A_2 | C |
| ATOM | 4391 | C8 | XP4 | F | 301 | −4.189 | 10.791 | 7.304 | 1.00 | 165.86 | A_2 | C |
| ATOM | 4392 | C9 | XP4 | F | 301 | −4.330 | 12.239 | 7.787 | 1.00 | 151.29 | A_2 | C |
| ATOM | 4393 | C10 | XP4 | F | 301 | −4.491 | 13.253 | 6.652 | 1.00 | 119.97 | A_2 | C |
| ATOM | 4394 | C11 | XP4 | F | 301 | −5.264 | 14.475 | 7.190 | 1.00 | 171.94 | A_2 | C |
| ATOM | 4395 | C12 | XP4 | F | 301 | −5.912 | 15.324 | 6.105 | 1.00 | 154.67 | A_2 | C |
| ATOM | 4396 | C13 | XP4 | F | 301 | −6.726 | 16.461 | 6.738 | 1.00 | 166.94 | A_2 | C |
| ATOM | 4397 | C14 | XP4 | F | 301 | −8.195 | 16.514 | 6.289 | 1.00 | 142.88 | A_2 | C |
| ATOM | 4398 | C15 | XP4 | F | 301 | −9.129 | 16.771 | 7.457 | 1.00 | 177.01 | A_2 | C |
| ATOM | 4399 | C16 | XP4 | F | 301 | −10.337 | 17.532 | 6.951 | 1.00 | 151.44 | A_2 | C |
| ATOM | 4400 | C17 | XP4 | F | 301 | −11.535 | 16.908 | 7.598 | 1.00 | 146.46 | A_2 | C |
| ATOM | 4401 | C2 | XP4 | G | 302 | −2.402 | 1.356 | 5.251 | 1.00 | 186.50 | | C |
| ATOM | 4402 | C3 | XP4 | G | 302 | −3.637 | 2.024 | 4.560 | 1.00 | 176.76 | | C |
| ATOM | 4403 | O5 | XP4 | G | 302 | −4.232 | 3.230 | 5.151 | 1.00 | 157.07 | | O |
| ATOM | 4404 | C4 | XP4 | G | 302 | −5.613 | 3.461 | 5.714 | 1.00 | 137.77 | | C |
| ATOM | 4405 | O6 | XP4 | G | 302 | −6.399 | 2.543 | 5.945 | 1.00 | 242.16 | | O |
| ATOM | 4406 | C5 | XP4 | G | 302 | −6.019 | 4.872 | 6.102 | 1.00 | 136.08 | | C |
| ATOM | 4407 | C6 | XP4 | G | 302 | −7.354 | 5.309 | 5.525 | 1.00 | 128.98 | | C |
| ATOM | 4408 | C7 | XP4 | G | 302 | −7.544 | 6.781 | 5.938 | 1.00 | 124.21 | | C |
| ATOM | 4409 | C8 | XP4 | G | 302 | −8.763 | 7.461 | 5.309 | 1.00 | 155.25 | | C |
| ATOM | 4410 | C9 | XP4 | G | 302 | −9.066 | 8.761 | 6.048 | 1.00 | 174.05 | | C |
| ATOM | 4411 | C10 | XP4 | G | 302 | −10.225 | 9.572 | 5.436 | 1.00 | 146.14 | | C |
| ATOM | 4412 | C11 | XP4 | G | 302 | −10.544 | 10.859 | 6.207 | 1.00 | 142.36 | | C |
| ATOM | 4413 | C12 | XP4 | G | 302 | −11.440 | 11.823 | 5.441 | 1.00 | 149.32 | | C |
| ATOM | 4414 | C13 | XP4 | G | 302 | −11.928 | 12.990 | 6.341 | 1.00 | 168.50 | | C |
| ATOM | 4415 | C14 | XP4 | G | 302 | −13.310 | 13.573 | 5.977 | 1.00 | 145.44 | | C |
| ATOM | 4416 | C15 | XP4 | G | 302 | −13.936 | 14.265 | 7.176 | 1.00 | 210.71 | | C |
| ATOM | 4417 | C16 | XP4 | G | 302 | −15.344 | 14.919 | 6.899 | 1.00 | 203.06 | | C |
| ATOM | 4418 | C17 | XP4 | G | 302 | −15.913 | 16.073 | 7.805 | 1.00 | 119.40 | | C |
| ATOM | 4419 | C3 | XP4 | H | 302 | 1.715 | 5.761 | 0.214 | 1.00 | 114.00 | | C |
| ATOM | 4420 | O5 | XP4 | H | 302 | 0.465 | 6.400 | −0.120 | 1.00 | 204.08 | | O |
| ATOM | 4421 | C4 | XP4 | H | 302 | −0.163 | 6.707 | −1.425 | 1.00 | 173.02 | | C |
| ATOM | 4422 | O6 | XP4 | H | 302 | 0.357 | 6.466 | −2.562 | 1.00 | 201.23 | | O |
| ATOM | 4423 | C5 | XP4 | H | 302 | −1.552 | 7.322 | −1.266 | 1.00 | 180.24 | | C |
| ATOM | 4424 | C6 | XP4 | H | 302 | −1.616 | 8.811 | −1.606 | 1.00 | 140.49 | | C |
| ATOM | 4425 | C7 | XP4 | H | 302 | −3.040 | 9.316 | −1.349 | 1.00 | 148.64 | | C |
| ATOM | 4426 | C8 | XP4 | H | 302 | −3.233 | 10.793 | −1.700 | 1.00 | 167.51 | | C |
| ATOM | 4427 | C9 | XP4 | H | 302 | −4.731 | 11.126 | −1.702 | 1.00 | 194.71 | | C |
| ATOM | 4428 | C10 | XP4 | H | 302 | −5.072 | 12.607 | −1.951 | 1.00 | 245.65 | | C |
| ATOM | 4429 | C11 | XP4 | H | 302 | −6.585 | 12.909 | −1.889 | 1.00 | 275.87 | | C |
| ATOM | 4430 | C12 | XP4 | H | 302 | −6.942 | 14.382 | −1.650 | 1.00 | 216.54 | | C |
| ATOM | 4431 | C13 | XP4 | H | 302 | −8.436 | 14.648 | −1.864 | 1.00 | 182.63 | | C |
| ATOM | 4432 | C14 | XP4 | H | 302 | −8.646 | 15.963 | −2.600 | 1.00 | 221.65 | | C |
| ATOM | 4433 | C15 | XP4 | H | 302 | −10.072 | 16.036 | −3.157 | 1.00 | 249.68 | | C |
| ATOM | 4434 | C16 | XP4 | H | 302 | −10.399 | 17.381 | −3.832 | 1.00 | 281.95 | | C |
| ATOM | 4435 | C17 | XP4 | H | 302 | −11.870 | 17.612 | −4.197 | 1.00 | 286.75 | | C |
| ATOM | 4436 | C3 | XP4 | I | 302 | 2.799 | 4.226 | 3.951 | 1.00 | 248.32 | | C |
| ATOM | 4437 | O5 | XP4 | I | 302 | 1.810 | 5.264 | 4.118 | 1.00 | 238.26 | | O |
| ATOM | 4438 | C4 | XP4 | I | 302 | 0.353 | 5.232 | 3.819 | 1.00 | 227.99 | | C |
| ATOM | 4439 | O6 | XP4 | I | 302 | −0.217 | 4.296 | 3.252 | 1.00 | 239.68 | | O |
| ATOM | 4440 | C5 | XP4 | I | 302 | −0.498 | 6.412 | 4.261 | 1.00 | 187.66 | | C |
| ATOM | 4441 | C6 | XP4 | I | 302 | −1.235 | 7.096 | 3.124 | 1.00 | 172.32 | | C |
| ATOM | 4442 | C7 | XP4 | I | 302 | −1.905 | 8.293 | 3.745 | 1.00 | 120.30 | | C |
| ATOM | 4443 | C8 | XP4 | I | 302 | −2.492 | 9.214 | 2.673 | 1.00 | 85.36 | | C |
| ATOM | 4444 | C9 | XP4 | I | 302 | −3.422 | 10.190 | 3.425 | 1.00 | 162.44 | | C |
| ATOM | 4445 | C10 | XP4 | I | 302 | −4.103 | 11.249 | 2.541 | 1.00 | 134.88 | | C |
| ATOM | 4446 | C11 | XP4 | I | 302 | −4.936 | 12.269 | 3.327 | 1.00 | 155.00 | | C |
| ATOM | 4447 | C12 | XP4 | I | 302 | −5.407 | 13.439 | 2.465 | 1.00 | 152.12 | | C |
| ATOM | 4448 | C13 | XP4 | I | 302 | −6.509 | 14.219 | 3.197 | 1.00 | 106.25 | | C |
| ATOM | 4449 | C14 | XP4 | I | 302 | −7.418 | 15.032 | 2.266 | 1.00 | 128.86 | | C |
| ATOM | 4450 | C15 | XP4 | I | 302 | −8.694 | 15.598 | 2.937 | 1.00 | 119.73 | | C |
| ATOM | 4451 | C16 | XP4 | I | 302 | −9.715 | 16.258 | 1.941 | 1.00 | 181.35 | | C |
| ATOM | 4452 | C17 | XP4 | I | 302 | −10.902 | 16.965 | 2.614 | 1.00 | 214.00 | | C |
| ATOM | 4453 | C3 | XP4 | K | 201 | −22.190 | 26.452 | −4.169 | 1.00 | 249.51 | | C |
| ATOM | 4454 | O5 | XP4 | K | 201 | −21.821 | 25.332 | −3.327 | 1.00 | 230.09 | | O |
| ATOM | 4455 | C4 | XP4 | K | 201 | −21.720 | 23.916 | −3.735 | 1.00 | 158.73 | | C |
| ATOM | 4456 | O6 | XP4 | K | 201 | −22.507 | 23.411 | −4.573 | 1.00 | 182.49 | | O |
| ATOM | 4457 | C5 | XP4 | K | 201 | −20.623 | 23.092 | −3.063 | 1.00 | 163.37 | | C |
| ATOM | 4458 | C6 | XP4 | K | 201 | −19.860 | 22.185 | −4.055 | 1.00 | 182.11 | | C |
| ATOM | 4459 | C7 | XP4 | K | 201 | −18.365 | 22.033 | −3.760 | 1.00 | 154.11 | | C |
| ATOM | 4460 | C8 | XP4 | K | 201 | −17.587 | 21.713 | −5.036 | 1.00 | 153.36 | | C |
| ATOM | 4461 | C9 | XP4 | K | 201 | −16.234 | 21.065 | −4.749 | 1.00 | 204.58 | | C |
| ATOM | 4462 | C10 | XP4 | K | 201 | −15.639 | 20.347 | −5.967 | 1.00 | 203.68 | | C |
| ATOM | 4463 | C3 | XP4 | L | 201 | −20.923 | 23.156 | 1.027 | 1.00 | 204.96 | | C |
| ATOM | 4464 | O5 | XP4 | L | 201 | −19.675 | 23.125 | 1.761 | 1.00 | 271.09 | | O |
| ATOM | 4465 | C4 | XP4 | L | 201 | −18.422 | 23.898 | 1.511 | 1.00 | 240.41 | | C |
| ATOM | 4466 | O6 | XP4 | L | 201 | −18.438 | 25.045 | 1.049 | 1.00 | 225.48 | | O |
| ATOM | 4467 | C5 | XP4 | L | 201 | −17.075 | 23.283 | 1.883 | 1.00 | 135.02 | | C |
| ATOM | 4468 | C6 | XP4 | L | 201 | −15.994 | 23.629 | 0.856 | 1.00 | 114.68 | | C |
| ATOM | 4469 | C7 | XP4 | L | 201 | −15.073 | 22.433 | 0.607 | 1.00 | 158.51 | | C |
| ATOM | 4470 | C8 | XP4 | L | 201 | −14.521 | 22.421 | −0.830 | 1.00 | 199.39 | | C |
| ATOM | 4471 | C9 | XP4 | L | 201 | −13.299 | 21.502 | −1.066 | 1.00 | 174.95 | | C |
| ATOM | 4472 | C10 | XP4 | L | 201 | −12.337 | 21.860 | −2.210 | 1.00 | 113.12 | | C |
| ATOM | 4473 | C3 | XP4 | M | 201 | −20.663 | 21.791 | 6.884 | 1.00 | 167.31 | | C |
| ATOM | 4474 | O5 | XP4 | M | 201 | −19.866 | 20.678 | 6.445 | 1.00 | 195.29 | | O |
| ATOM | 4475 | C4 | XP4 | M | 201 | −18.966 | 20.729 | 5.227 | 1.00 | 181.13 | | C |
| ATOM | 4476 | O6 | XP4 | M | 201 | −19.181 | 21.440 | 4.214 | 1.00 | 216.12 | | O |
| ATOM | 4477 | C5 | XP4 | M | 201 | −17.732 | 19.829 | 5.231 | 1.00 | 210.18 | | C |
| ATOM | 4478 | C6 | XP4 | M | 201 | −16.462 | 20.562 | 4.805 | 1.00 | 255.23 | | C |
| ATOM | 4479 | C7 | XP4 | M | 201 | −15.193 | 20.167 | 5.584 | 1.00 | 110.44 | | C |
| ATOM | 4480 | C8 | XP4 | M | 201 | −14.082 | 21.260 | 5.492 | 1.00 | 156.12 | | C |
| ATOM | 4481 | C9 | XP4 | M | 201 | −12.667 | 20.822 | 5.928 | 1.00 | 238.25 | | C |
| ATOM | 4482 | C10 | XP4 | M | 201 | −11.534 | 21.757 | 5.463 | 1.00 | 108.10 | | C |

The skilled artisan will recognize the interchangeability of various features from different embodiments. Similarly, the various features and steps discussed above, as well as other known equivalents for each such feature or step, can be mixed and matched by one of ordinary skill in this art to obtain crystals, crystal structures, or structure-based rational ligand design methods in accordance with principles and invention described herein. Although the disclosure has been provided in the context of certain embodiments and examples, it will be understood by those skilled in the art that the disclosure extends beyond the specifically described embodiments to other alternative embodiments and/or uses and obvious modifications and equivalents thereof. Accordingly, the disclosure is not intended to be limited by the specific disclosures of embodiments herein.

What is claimed is:

1. A crystal comprising a HpUreI protein, wherein atomic coordinates of the HpUreI protein in the crystal correspond, at least at a 70% level, to those set forth in Table 4.

2. The crystal of claim 1, wherein the crystal comprises approximately the following cell constants: a=123 Å, b=123 Å, c=140 Å and an approximate space group of P4₂2₁2.

3. A method, for identifying a ligand of HpUreI protein, comprising:
searching a molecular structure library with a crystal structure of a HpUreI protein; and
identifying, from the molecular structure library, a ligand that binds the HpUreI protein, wherein the crystal structure comprises atomic coordinates of the HpUreI protein that correspond, at least at a 70% level, to those set forth in Table 4, and wherein the ligand comprises a 3-dimensional structure that binds to the crystal structure in an operatively fitting manner and thereby modulates a urea channel activity of the HpUreI protein.

4. The method of claim 3, wherein the molecular structure library consists of a small molecule, an antibody, a peptide, a protein, a DNA sequence, and an RNA sequence.

5. The method of claim 3, wherein the modulation consists of a decrease in the urea channel activity.

6. The method of claim 3, wherein the modulation consists of an increase the urea channel activity.

7. The method of claim 3, further comprising determining an efficiency with which the ligand modulates the urea channel activity by at least one of computational evaluation and experimental evaluation.

8. The method of claim 3, further comprising determining an affinity with which the ligand binds HpUreI protein by at least one of computational evaluation and experimental evaluation.

9. The method of claim 3, wherein the ligand binds HpUreI protein with an affinity selected from the group of ranges consisting of micromolar, nanomolar, picomolar, and femtomolar.

10. The method of claim 3, further comprising determining, by x-ray diffraction, the atomic coordinates of the HpUreI protein in a crystal thereof.

11. The method of claim 10, wherein the crystal of HpUreI protein comprises approximately the following cell constants: a=123 Å, b=123 Å, c=140 Å and an approximate space group of P4₂2₁2.

12. A method, for forming an HpUreI protein crystal, comprising:
exposing a crystallization volume comprising a precipitant solution and an HpUreI protein to conditions that promote HpUreI protein crystal formation; and
forming, from the crystallization volume, a crystal comprising the HpUreI protein, wherein atomic coordinates of the HpUreI protein in the crystal are determinable by x-ray diffraction and correspond, at least at a 70% level, to those set forth in Table 4.

13. The method of claim 12, wherein the crystal comprises approximately the following cell constants: a=123 Å, b=123 Å, c=140 Å and a space group of approximately P4₂2₁2.
